(19) United States
(12) Patent Application Publication
(10) Pub. No.: US 2026/0263602 A1
(43) Pub. Date: Sep. 10, 2026

(54) COMPOUNDS TARGETING DMPK FOR THE TREATMENT OF MYOTONIC DYSTROPHY

(71) Applicant: Novartis AG, Basel (CH)

(72) Inventors: Charles Robert ALLERSON, San Diego, CA (US); Riann Jenay EGUSQUIZA, San Diego, CA (US); Zhihong HUANG, San Diego, CA (US); Arthur T. SUCKOW, San Diego, CA (US)

(21) Appl. No.: 19/461,064

(22) Filed: Jan. 27, 2026

Related U.S. Application Data
(60) Provisional application No. 63/751,129, filed on Jan. 29, 2025.

(51) Int. Cl. A61K 47/54; A61K 9/00; A61K 31/712; A61K 31/713; A61P 21/00; C12N 15/113

(52) U.S. Cl. CPC A61K 47/542; A61K 9/0019; A61K 31/712; A61K 31/713; A61P 21/00; C12N 15/1137; C12N 2310/11; C12N 2310/14; C12N 2310/321; C12N 2310/322; C12N 2310/351; C12Y 207/11001

(57) ABSTRACT

The present disclosure relates to double stranded RNAi agents and methods for the treatment of myotonic dystrophy type 1. The double stranded RNAi agents may have a structure of formula (I),

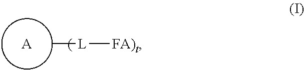

wherein A, L and FA are as described herein.

### CROSS-REFERENCE TO RELATED APPLICATIONS

This application claims the benefit of U.S. Provisional Application No. 63/751,129, filed Jan. 29, 2025, which are incorporated by reference herein in their entirety.

### REFERENCE TO AN ELECTRONIC SEQUENCE LISTING

The contents of the electronic sequence listing (PAT059785-WO-PCT_SL.xml; Size: 10,120,045 bytes; and Date of Creation: Jan. 26, 2026) are hereby incorporated by reference in their entirety.

### FIELD

The present disclosure relates to compounds and methods for the treatment of myotonic dystrophy type 1. More specifically, the present disclosure relates to RNA interference (RNAi) agents, e.g., double stranded RNAi agents, for inhibition of DMPK gene expression, compositions comprising DMPK RNAi agents, and their use in the treatment of myotonic dystrophy type 1.

### BACKGROUND

Myotonic dystrophy type I (DM1) is an inherited, progressive neuromuscular disorder that is the most common adult-onset muscular dystrophy. While DM1 primarily affects muscle tissue, resulting in progressive muscle atrophy and weakness and myotonia, symptoms also occur in the heart, brain, gastrointestinal tract, and endocrine and respiratory systems. DM1 is caused by expansion of a CTG repeat in the 3′-untranslated region of the dystrophia myotonica protein kinase (DMPK) gene. In the resultant DMPK transcript, the repeat expansion forms hairpin structures that aggregate in the nucleus in the form of nuclear foci and recruit RNA binding proteins, preventing these proteins from performing their normal functions. For example, the DMPK hairpin structure sequesters muscleblind-like splicing regulator 1 (MBNL1) and prevents it from performing its normal function of regulating RNA alternative splicing, which results in splice defects in several transcripts. The length of the CTG repeat expansion is associated with disease severity, with larger repeat expansions causing earlier onset and increased severity of disease.

While a small number of potential therapies are being evaluated in clinical trials, an effective treatment for DM1 has yet to be identified. Accordingly, there remains an unmet medical need for therapeutic agents for the treatment of DM1.

### SUMMARY

There is a need for novel RNA interference (RNAi) agents, e.g., double stranded RNAi (dsRNAi) agents, such as small interfering RNAs (siRNAs), that are able to efficiently inhibit the expression of a dystrophia myotonica protein kinase (DMPK) gene, particularly in vivo.

In general, the present disclosure features dsRNAi agents, compositions that include such dsRNAi agents, and methods for inhibiting expression of a DMPK gene in vitro and/or in vivo using the dsRNAi agents and compositions that include the dsRNAi agents described herein. The dsRNAi agents described herein are able to efficiently decrease, inhibit, or silence expression of a DMPK gene.

The described dsRNAi agents can be used in methods for therapeutic treatment of symptoms and diseases such as myotonic dystrophy type 1. The methods disclosed herein include the administration of one or more dsRNAi agents to a subject, e.g., a human or animal subject, using any suitable methods known in the art, such as for example, subcutaneous injection, intramuscular injection, or intravenous administration.

In one aspect, the disclosure features dsRNAi agents for inhibiting expression of a DMPK gene, wherein the RNAi agent includes a sense strand (also referred to as a passenger strand) and an antisense strand (also referred to as a guide strand). The sense strand and the antisense strand can be partially, substantially, or fully complementary to each other. The length of the RNAi agent sense strands described herein each can be 10 to 30 nucleotides in length. The length of the RNAi agent antisense strands described herein each can be 10 to 30 nucleotides in length. In some embodiments, the sense and antisense strands are independently 17 to 25 nucleotides in length. The sense and antisense strands can be either the same length or different lengths. In some embodiments, the sense and antisense strands are independently 19 to 23 nucleotides in length. In some embodiments, the sense strand is 19 nucleotides in length. In some embodiments, the antisense strand is 21 nucleotides in length. In some embodiments, the antisense strands are independently 17, 18, 19, 20, 21, 22, 23, 24, or 25 nucleotides in length. In some embodiments, the sense strands are independently 15, 16, 17, 18, 19, 20, 21, 22, or 23 nucleotides in length. The dsRNAi agents described herein, upon delivery to a cell expressing DMPK, such as a skeletal muscle cell (a skeletal myofiber), inhibit the expression of one or more DMPK gene transcripts in vivo and/or in vitro.

The dsRNAi agents disclosed herein target a DM1 protein kinase (DMPK) gene (see, e.g., SEQ ID NO:1, Homo sapiens transcript variant 1). In some embodiments, the RNAi agents disclosed herein target a portion of a DMPK gene having the sequence of any of the sequences disclosed in Table 1.

In another aspect, the disclosure features pharmaceutical compositions that include a dsRNAi agent that is able to efficiently decrease expression of a DMPK gene. The pharmaceutical compositions that include the dsRNAi agent described herein can be administered to a subject, such as a human or animal subject, for the treatment of symptoms and diseases that can be ameliorated at least in part by a reduction in DMPK mRNA levels, and more specifically a reduction in mutant DMPK-CUG mRNA levels. The pharmaceutical compositions described herein include an RNAi agent capable of inhibiting the expression of a DMPK gene and at least one pharmaceutically acceptable excipient. In certain embodiments, the dsRNAi agents and compositions described herein inhibit mutant DMPK or expanded CUG (CUGexp) DMPK.

Examples of dsRNAi agent sense strands and antisense strands that can be used in a dsRNAi agent are provided in Tables 1 and 2. Examples of dsRNAi agent duplexes are provided in Tables 1, 2, 8, 9 and 11.

In one aspect, provided herein is a double stranded RNAi (dsRNAi) agent, or a pharmaceutically acceptable salt thereof, of formula (I)

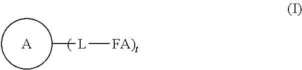

wherein:
A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
L is a linker;
FA is a fatty acid moiety capable of facilitating delivery of the double stranded nucleic acid into a cell; and
t is an integer from 1 to 5.

In another aspect, provided herein a dsRNAi agent of the structure of formula (II):

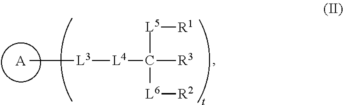

or a pharmaceutically acceptable salt thereof,
wherein A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length, or
wherein A is a double stranded nucleic acid comprising a sense strand and an antisense strand, the antisense strand forming a duplex with the sense strand, wherein each of the antisense strand and the sense strand is independently 17 to 25 nucleotides in length, wherein the nucleotide sequence of the antisense strand is at least 90% complementary to SEQ ID NO: 1, and the nucleotide sequence of the sense strand has no more than two mismatches to the nucleotide sequence of the antisense strand,
L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is -L^{5A}-L^{5B}-L^{5C}-L^{5D}-L^{5E}-;
L⁶ is -L^{6A}-L^{6B}-L^{6C}-L^{6D}-L^{6E}-;
L^{5A}, L^{5B}, L^{5C}, L^{5D}, L^{5E} L^{6A}, L^{6B}, L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹ and R² are independently unsubstituted C₁-C₂₅ alkyl or unsubstituted C₁-C₂₅ alkenyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
t is an integer from 1 to 5.

In embodiments, L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted C₁-C₂₀ alkylene, substituted or unsubstituted 2 to 20 membered heteroalkylene, substituted or unsubstituted C₃-C₁₀ cycloalkylene, substituted or unsubstituted 2 to 20 membered heterocycloalkylene, substituted or unsubstituted C₆-C₁₂ arylene, or 5 to 12 membered heteroarylene, wherein the C₁-C₂₀ alkylene, 2 to 20 membered heteroalkylene, C₃-C₁₀ cycloalkylene, 2 to 20 membered heterocycloalkylene, C₆-C₁₂ arylene, and 5 to 12 membered heteroarylene, are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen;
L⁵ is -L^{5A}-L^{5B}-L^{5C}-L^{5D}-L^{5E}-;
L⁶ is -L^{6A}-L^{6B}-L^{6C}-L^{6D}-L^{6E}-;
L^{5A}, L^{5B}, L^{5C}, L^{5D}, L^{5E}, L^{6A}, L^{6B} L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted C₁-C₂₀ alkylene, substituted or unsubstituted 2 to 20 membered heteroalkylene, substituted or unsubstituted C₃-C₁₀ cycloalkylene, substituted or unsubstituted 2 to 20 membered heterocycloalkylene, substituted or unsubstituted C₆-C₁₂ arylene or substituted or unsubstituted 5 to 12 membered heteroarylene, wherein the C₁-C₂₀ alkylene, 2 to 20 membered heteroalkylene, C₃-C₁₀ cycloalkylene, 2 to 20 membered heterocycloalkylene, C₆-C₁₂ arylene, and 5 to 12 membered heteroarylene, are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen;
R¹ and R² are independently unsubstituted C₁-C₂₅ alkyl or unsubstituted C₁-C₂₅ alkenyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted C₁-C₂₀ alkyl, substituted or unsubstituted 2 to 20 membered heteroalkyl, substituted or unsubstituted C₃-C₁₀ cycloalkyl, substituted or unsubstituted 2 to 20 membered heterocycloalkyl, substituted or unsubstituted C₆-C₁₂ aryl, or substituted or unsubstituted 5 to 12 membered heteroaryl, wherein the C₁-C₂₀ alkyl, 2 to 20 membered heteroalkyl, C₃-C₁₀ cycloalkyl, 2 to 20 membered heterocycloalkyl, C₆-C₁₂ aryl, and 5 to 12 membered heteroaryl, are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen; and
t is an integer from 1 to 5.

In embodiments, L³ is independently selected from:
(i) a bond;

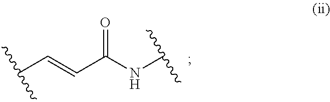 [SMILES: */C=C/C(=O)N*]

(iii) —OP(O)(OH)O—;
(iv) —OP(S)(OH)O—; and
(v) —O—;
L⁴ is independently selected from:
(i) substituted or unsubstituted heteroalkylene;
(ii) -L⁷-NH—C(O)— or -L⁷-C(O)—NH—, wherein L⁷ is substituted or unsubstituted alkylene;

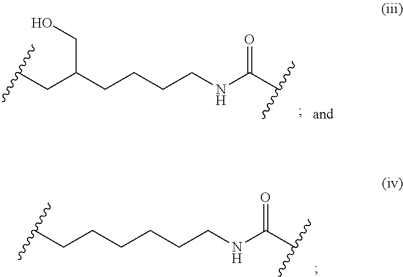 [SMILES: *CC(CO)CCCCNC(*)=O ; *CCCCCCNC(*)=O]

L⁵ is substituted or unsubstituted 2 to 20 membered heteroalkylene, or —
L^{5A}L^{5B}L^{5C}L^{5D}L^{5E}-,
wherein L^{5A} is independently unsubstituted C₁-C₈ alkylene; L^{5B} is independently —NHC(O)—; L^{5C} is independently a bond; L^{5D} is independently a bond or unsubstituted C₁-C₈ alkylene; and L^{5E} is independently a bond or —NHC(O)—;
L⁶ is —NHC(O)—;
R¹ and R² are independently unsubstituted C₁₁-C₁₇ alkyl;
R³ is hydrogen; and
t is 1.

In another aspect, provided herein is a dsRNAi agent, or a pharmaceutically acceptable salt thereof, of formula (Ia)

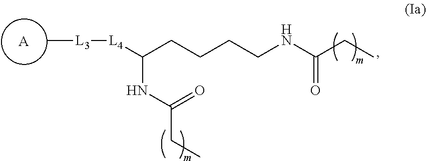

wherein:
each m, at each occurrence, is independently an integer from 10 to 18;
-L³-L⁴- is defined according to any one of the aspects or embodiments disclosed herein, e.g., according to any one of enumerated embodiments 1 to 185; and A is defined according to any one of the aspects or embodiments disclosed herein, e.g., according to any one of enumerated embodiments 1 to 185.

In some embodiments, the dsRNAi agent has formula (Ib)

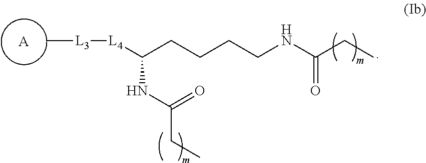

In some embodiments, the dsRNAi agent has formula (Ic)

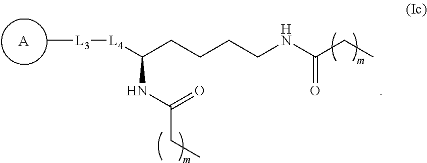

In another aspect, provided herein is a dsRNAi agent, or a pharmaceutically acceptable salt thereof, of formula (Id)

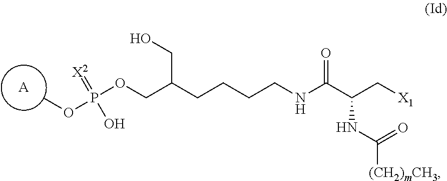

wherein
X² is selected from O and S;
X¹ is

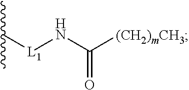

L¹ is —(CH₂)ₙ—, —(CH₂)ₙL₂(CH₂)ₙ—, or a bond;
L² is —C(═O)NH—, —C(═O)O—, —OC(═O)O—, —NHC(═O)O—, —NHC(═O)NH—, —C(═S)NH—, —C(═O)S—, —NH—, O (oxygen), or S (sulfur), and wherein each m is independently an integer from 10 to 18 and wherein each n is independently an integer from 1 to 6; and
A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length, or
wherein A is a double stranded nucleic acid comprising a sense strand and an antisense strand, the antisense strand forming a duplex with the sense strand, wherein each of the antisense strand and the sense strand is independently 17 to 25 nucleotides in length, wherein the nucleotide sequence of the antisense strand is at least 90% complementary to SEQ ID NO: 1, and the nucleotide sequence of the sense strand has no more than two mismatches to the nucleotide sequence of the antisense strand.

Additional embodiments of A are defined according to any one of the aspects or embodiments disclosed herein, e.g., according to any one of enumerated embodiments 1 to 185.

In some embodiments, the nucleotide sequence of the antisense strand comprises at least 17 to 23 contiguous nucleotides of a nucleotide sequence selected from any one of SEQ ID Nos: 294 to 584.

In some embodiments, the nucleotide sequence of the sense strand comprises at least 17 to 23 contiguous nucleotides of a nucleotide sequence selected from any one of SEQ ID Nos: 3 to 293.

In some embodiments, the nucleotide sequence of the antisense strand comprises at least 17 to 23 contiguous nucleotides of a nucleotide sequence selected from any one of SEQ ID Nos: 294 to 584, and nucleotide sequence of the sense strand comprises at least 17 to 23 contiguous nucleotides of a nucleotide sequence selected from any one of SEQ ID Nos: 3 to 293.

In some embodiments, the double stranded nucleic acid A is modified.

In some embodiments, each nucleotide of the sense strand and antisense strand is a modified nucleotide.

In another aspect, provided herein is a dsRNAi agent comprising:
(i) an antisense strand having a nucleotide sequence of SEQ ID NO: 1268; and
(ii) a sense stand strand having a nucleotide sequence of SEQ ID NO: 1267.

In another aspect, provided herein is a dsRNAi agent comprising:
(i) an antisense strand having a nucleotide sequence of SEQ ID NO: 1312; and
(ii) a sense stand strand having a nucleotide sequence of SEQ ID NO: 1311.

In another aspect, provided herein is a dsRNAi agent comprising:
(i) an antisense strand having a nucleotide sequence of SEQ ID NO: 1346; and
(ii) a sense stand strand having a nucleotide sequence of SEQ ID NO: 1345.

In another aspect, provided herein is a dsRNAi agent comprising:
(i) an antisense strand having a nucleotide sequence of SEQ ID NO: 1348; and
(ii) a sense stand strand having a nucleotide sequence of SEQ ID NO: 1347.

In another aspect, provided herein is a dsRNAi agent comprising:
(i) an antisense strand having a nucleotide sequence of SEQ ID NO: 1352; and
(ii) a sense stand strand having a nucleotide sequence of SEQ ID NO: 1365.

In another aspect, provided herein is a dsRNAi agent comprising:
(i) an antisense strand having a nucleotide sequence of SEQ ID NO: 1352; and
(ii) a sense stand strand having a nucleotide sequence of SEQ ID NO: 1351.

In yet another aspect, provided is a dsRNAi agent, or a pharmaceutically acceptable salt thereof, comprising:
(i) an antisense strand having a nucleotide sequence of

(SEQ ID NO: 1352)
5′-X033U1027p001C007p001A004p000A004p000U004p000
A007p000A004p000A004p000U004p000A004p000U004p000
C004p000C004p000A007p000A004p000A007p000C004p000
C004p000G004p001C004p001C004-3′;

and
(ii) a sense stand strand having a nucleotide sequence of

(SEQ ID NO: 1351)
5′-C004p001G004p001G004p000U004p000U007p000U004
p000G007p000G007p000A007p000U004p000A004p000U004
p000U004p000U004p000A004p000U004p000U004p001G004
p001U004p000X2140-3′,

wherein p000 is a phosphate internucleoside linkage, p001 is a phosphorothioate internucleoside linkage, X033U1027 is 5′-(E)-vinylphosphonate-2′-O-methyluridine, C004 is 2′—O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

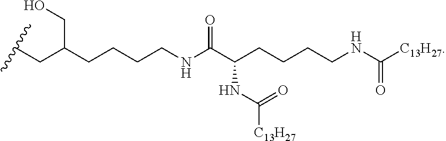

In an aspect, provided is a pharmaceutical composition including a compound as described herein, or a pharmaceutically acceptable salt thereof, and a pharmaceutically acceptable diluent.

In another aspect, the disclosure provides methods for delivering dsRNAi agents to skeletal muscle cells in a subject, such as a mammal, e.g., a human subject, in vivo. Also described herein are compositions for use in such methods.

In an aspect, the disclosure provides a method for inhibiting the expression of DMPK in a subject. In another aspect, the disclosure provides a method for treating myotonic dystrophy type I in a subject. The methods include administering to the subject a therapeutically effective amount of a compound described herein, or a pharmaceutically acceptable salt thereof, or a pharmaceutical composition as described herein.

In an aspect, the disclosure provides a method for inhibiting the expression of DMPK mRNA in a cell, the method comprising contacting a dsRNAi agent as described herein, or a pharmaceutically acceptable salt thereof, with the cell.

In an aspect, the disclosure provides a method for reducing or preventing aggregation of one or more DMPK mRNA transcripts within a cell, the method comprising contacting a dsRNAi agent as described herein, or a pharmaceutically acceptable salt thereof, with the cell.

In another aspect, the disclosure provides a method for inhibiting the expression of DMPK in a subject in need thereof, the method comprising administering to the subject a therapeutically effective amount of a dsRNAi agent as described herein, or a pharmaceutically acceptable salt thereof, or a pharmaceutical composition as described herein.

In another aspect, the disclosure provides a method for treating myotonic dystrophy type I (DM1) in a subject in need thereof, the method comprising administering to the subject a therapeutically effective amount of a dsRNAi agent as described herein, or a pharmaceutically acceptable salt thereof, or a pharmaceutical composition as described herein.

In another aspect, the disclosure provides a method for delaying the onset of DM1 in a subject in need thereof, the method comprising administering to the subject a therapeutically effective amount of a dsRNAi agent as described herein, or a pharmaceutically acceptable salt thereof, or a pharmaceutical composition as described herein.

In another aspect, the disclosure provides a method for treating DM1 in a subject in need thereof, the method comprising administering to the subject a therapeutically effective amount of a dsRNAi agent as described herein, or a pharmaceutically acceptable salt thereof, or a pharmaceutical composition as described herein, wherein the administration of the dsRNAi agent relieves at least one symptom of DM1 in at least one muscle.

In another aspect, the disclosure provides a method for treating DM1 in a subject in need thereof, the method comprising systemically administering to the subject a therapeutically effective amount of a dsRNAi agent as disclosed herein, or a pharmaceutically acceptable salt thereof, or a pharmaceutical composition as described herein, wherein the administration of the dsRNAi agent relieves at least one symptom of DM1 in at least one muscle.

In embodiments, the pharmaceutically acceptable salt is a sodium salt.

In embodiments, the at least one symptom of DM1 is myotonia.

In embodiments, the at least one symptom of DM1 is aggregation of musclebind-like-1 (MBNL-1) protein in ribonuclear foci within myonuclei.

In embodiments, the at least one symptom of DM1 is abnormal splicing of at least one RNA transcript in muscle cells.

In embodiments, the muscle is selected from the group consisting of skeletal muscle, smooth muscle, and cardiac muscle.

In embodiments, the administration is intravenous administration or subcutaneous administration.

The above aspects and embodiments therein can be combined. Other objects, features, advantages and aspects of the present disclosure will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosure will become readily apparent to those skilled in the art from reading the following.

### BRIEF DESCRIPTION OF THE DRAWINGS

FIG. 1 shows in vitro test results of unconjugated siRNAs for inhibiting expression of DMPK in human skeletal muscle cells (HSkMCs) at 10 nM dose.

FIG. 2 shows the selected in vitro test results of unconjugated siRNAs selected from FIG. 1, and the selected hits having below 46% expression of DMPK in human skeletal muscle cells (HSkMCs) at 10 nM dose are shown.

FIGS. 3A-3B show inhibition of DMPK mRNA expression by siRNA conjugated with DTx-01-32 and DTx-01-08, respectively, scaffold in muscle cells in quadricep (FIG. 3A) and transversus abdominis (FIG. 3B).

FIG. 4 shows in vivo test results of conjugated siRNAs for inhibiting expression of DMPK in HSkMCs at 333 nM dose.

FIG. 5 shows in vivo test results of the selected conjugated siRNAs for inhibiting expression of DMPK in gastrocnemius of hemizygous DMSXL mice at a dose of 10 mg/kg and 30 mg/kg.

FIG. 6 shows in vivo test results of the selected conjugated siRNAs for inhibiting expression of DMPK in heart of hemizygous DMSXL mice at a dose of 10 mg/kg and 30 mg/kg.

FIG. 7 shows in vivo test results of the selected conjugated siRNAs for inhibiting expression of DMPK in tongue of hemizygous DMSXL mice at a dose of 10 mg/kg and 30 mg/kg.

FIG. 8 shows in vivo test results of the selected conjugated siRNAs for inhibiting expression of DMPK in diaphragm of hemizygous DMSXL mice at a dose of 10 mg/kg and 30 mg/kg.

FIG. 9 shows in vivo results of human DMPK knockdown by DT-001068 and DT-002211 at a dose of 10 mg/kg and 30 mg/kg in homozygous DMSXL mice and the mRNA and average percentage expression for human DMPK mRNA in gastrocnemius and heart tissues are shown.

FIG. 10 shows in vivo results of human DMPK knockdown by several conjugated siRNA at a dose of 10 mg/kg and 30 mg/kg in NHP and average percentage expression of human DMPK mRNA in rectus femoris muscle, gastrocnemius, diaphragm, and heart are shown.

FIG. 11 shows potent, dose dependent hDMPK mRNA knockdown by several conjugated siRNAs in DM1 patient myotubes.

FIG. 12 shows splicing correction efficacy (composite splice index) of DT-001068, DT-002211, DT-003035, DT-03036, and DT-003038 at a dose of 30 mg/kg in homozygous DMSXL mice and composite splice indexes in heart and gastrocnemius tissues are shown.

FIG. 13 shows splicing correction efficacy (composite splice index) of DT-001068, DT-002211, DT-003035, DT-003036, and DT-003038 at a dose of 30 mg/kg in homozygous DMSXL mice and composite splice indexes in diaphragm and tongue tissues are shown.

FIG. 14 shows efficacy in Homozygous DMSXL of DT-001068, DT-002211, DT-003035, DT-03036, and DT-003038 at a dose of 30 mg/kg in homozygous DMSXL mice and total RNA foci area (CUG foci area) data collected from heart and tibialis anterior are shown.

FIG. 15 shows splicing correction efficacy (fold change compared to vehicle) of DT-001068, DT-002211, DT-003035, DT-03036, and DT-003038 at a dose of 30 mg/kg in DM1 patient myotubes.

FIG. 16 shows dose dependent reduction of CUG-MBNL1 co-foci reduction by DT-001068, DT-002211, DT-003035, DT-003036, and DT-003038 at dose dependent reduction in DM1 patient myotubes.

FIG. 17 shows a tissue exposure of DT-002211, DT-003035, DT-003036, and DT-003038 at a dose of 30 mg/kg in gastrocnemius of homozygous DMSXL mice.

FIG. 18 shows a tissue exposure of DT-001068, DT-002211, DT-003035, DT-003036, and DT-003038 at a dose of 30 mg/kg in gastrocnemius of NHP.

FIG. 19 shows an in vivo comparison of human DMPK knockdown by DT-003037 and DT-003038 at a dose of 10 mg/kg and 30 mg/kg in hemizygous DMSXL mice in gastrocnemius heart, diaphragm and tongue tissues at 14 days.

FIG. 20 shows an in vivo comparison of human DMPK knockdown by DT-003037 and DT-003038 at a dose of 10 mg/kg and 30 mg/kg in hemizygous DMSXL mice in gastrocnemius heart, diaphragm and tongue tissues at 28 days.

The present disclosure is directed to dsRNAi agents for silencing, reducing, or inhibiting, expanded repeat DMPK mRNA for the treatment of mytonic dystrophy type I (DM1).

### DETAILED DESCRIPTION

### Definitions

Unless defined otherwise, all technical terms, scientific terms, abbreviations, chemical structures, and chemical formulae used herein have the same meaning as is commonly understood by one of ordinary skill in the art. The chemical structures and formulae set forth herein are constructed according to the standard rules of chemical valency known in the chemical arts. All patents, applications, published applications, and other publications referenced herein are incorporated by reference in their entirety unless stated otherwise. Unless otherwise indicated, conventional methods of mass spectroscopy, NMR, HPLC, protein chemistry, biochemistry, recombinant DNA techniques, and pharmacology are employed.

Unless specified otherwise, the terms “compound(s) of the present disclosure”, “compound(s) of the disclosure”, compound(s) as disclosed herein, and compound(s) as described herein, and the like, refer to a double stranded RNA, e.g., DMPK dsRNA or dsRNAi agent as disclosed herein, including compounds of formulae (I), (II), (Ia), (Ib), (Ic), (Id), (Ie), (If), and (Ig), exemplified compounds, salts thereof, particularly pharmaceutically acceptable salts thereof, hydrates, solvates, prodrugs, as well as all stereoisomers (including diastereoisomers and enantiomers), rotamers, tautomers, and isotopically labeled compounds (including deuterium substitutions), as well as inherently formed moieties. In embodiments, the double stranded RNA may be conjugated with a ligand, e.g., a linker-fatty acid moiety as disclosed herein. The double stranded RNA may be in free form or in pharmaceutically acceptable salt form. The double stranded RNA may be provided as a pharmaceutical composition.

In the groups, radicals, or moieties defined below, the number of carbon atoms is often specified preceding the group, for example, C₁₋₁₀ alkyl means an alkyl group or radical having 1 to 10 carbon atoms. Furthermore, the use of a term designating a monovalent radical where a divalent radical is appropriate shall be construed to designate the respective divalent radical and vice versa. Unless otherwise specified, conventional definitions of terms control and conventional stable atom valences are presumed and achieved in all formulas and groups.

Although the sequence listing for a nucleic acid or an oligonucleotide in the disclosure identifies each nucleotide sequence as either “RNA” or “DNA” as required, in practice, those sequences may be modified with a combination of chemical modifications specified herein. One of skill in the art will readily appreciate that in the sequence listing, such designation as “RNA” or “DNA” to describe modified nucleotides is somewhat arbitrary. For example, a nucleic acid provided herein comprising a nucleotide comprising a 2′-O-methyl sugar moiety and a thymine base may be described as a DNA residue in the sequence listing, even though the nucleotide is modified and is not a naturally-occurring DNA nucleotide.

Furthermore, use of the term “including” as well as other forms, such as “include”, “includes,” and “included,” is not limiting. As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

As used herein, the term “a,” “an,” “the” and similar terms used in the context of the present disclosure (especially in the context of the claims) are to be construed to cover both the singular and plural unless otherwise indicated herein or clearly contradicted by the context.

Unless otherwise indicated, all numbers, values, and/or expressions, including referring to nucleotide lengths, inhibition, activities, dosages, contents, and formulations used herein are to be understood as modified in all instances by the term “about” as such numbers are inherently approximations that are reflective of, among other things, the various uncertainties of measurement encountered in obtaining such values. Further, unless specifically stated or obvious from context, as used herein, the term “about” is understood as within a range of normal tolerance in the art, for example within 2 standard deviations of the mean. “About” may be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

Throughout the disclosure, nucleotide positions or coordinates are relative to the beginning (5′ end) of the reference transcript.

As used herein, the modification codes have the following meanings: “P000” denotes a phosphate internucleoside linkage; “P001” denotes a phosphorothioate internucleoside linkage; “X033U1027” denotes 5′-(E)-vinylphosphonate-2′-O-methyluridine; “C004” denotes 2′-O-methylcytidine; “C007” denotes 2′-fluorocytidine; “A004” denotes 2′-O-methyladenosine; “A007” denotes 2′-fluoroadenosine; “U004” denotes 2′-O-methyluridine; “U007” denotes 2′-fluorouridine; “G004” denotes 2′-O-methylguanosine; “G007” denotes 2′-fluoroguanosine; and “X2140” denotes the radical N,N-((5S)-6-((6-hydroxy-S—(λ³-methyl)hexyl)amino)-6-oxohexane-1,5-diyl)ditetradecanamide, having the structural formula

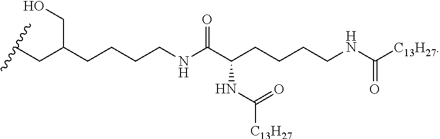

“Administration” means providing a pharmaceutical agent or composition to a subject, and includes administration performed by a medical professional and self-administration. In some embodiments, administration is intravenous administration. In some embodiments, administration is subcutaneous administration.

As used herein, the term “amino acid scaffold” refers to a structural framework comprising or derived from one or more amino acids. In an embodiment, the amino acid scaffold is a chiral center-defined structure with a carbon atom located between amino and carboxylic acid moieties. The amino acid scaffold may include molecules such as arginine, histidine, lysine, aspartic acid, glutamic acid, serine, threonine, asparagine, glutamine, cysteine, glycine, proline, alanine, valine, isoleucine, leucine, methionine, phenylalanine, tyrosine, and tryptophan. The term “alpha amino acid scaffold” refers to a structural framework comprising or derived from one or more alpha amino acids, accordingly. In an embodiment, the alpha amino acid scaffold comprises or is derived from lysine.

The term “antisense strand”, as used herein, refers to an oligonucleotide, e.g., RNA, of an siRNA or a dsRNAi that is complementary, e.g., partially, substantially, or fully complementary, to the target mRNA and is incorporated into the RNA-induced silencing complex (RISC) to direct gene silencing in a sequence-specific manner through the RNA interference pathway. An antisense strand may also be referred to as the “guide strand”. In some embodiments, the antisense strand may have a length from 15-30 nt, 15-26 nt, 15-23 nt, 15-22 nt, 15-21 nt, 15-20 nt, 15-19 nt, 15-18 nt, 15-17 nt, 18-30 nt, 18-26 nt, 18-23 nt, 18-22 nt, 18-21 nt, 18-20 nt, 19-30 nt, 19-26 nt, 19-23 nt, 19-22 nt, 19-21 nt, 19-20 nt, 19 nt, 20-30 nt, 20-26 nt, 20-25 nt, 20-24 nt, 20-23 nt, 20-22 nt, 20-21 nt, 20 nt, 21-30 nt, 21-26 nt, 21-25 nt, 21-24 nt, 21-23 nt, 21-22 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, or 36 nt.

The term “associated” or “associated with” in the context of a substance or substance activity or function associated with a disease, e.g. a DMPK associated disease, means that the disease is caused by (in whole or in part), or a symptom of the disease is caused by (in whole or in part) the substance or substance activity or function, e.g., DMPK activity or function. Thus, as used herein, what is described as “being associated” with a disease, if a causative agent, could be a target for treatment of the disease.

A “beta-D-deoxyribonucleoside” means a naturally occurring nucleoside monomer of DNA.

A “beta-D-ribonucleoside” means a naturally occurring nucleoside monomer of RNA.

“Bicyclic sugar” means a modified sugar moiety comprising a linkage connecting the 2′-carbon and 4′-carbon of the pentafuranosyl sugar, resulting in a bicyclic structure. Non-limiting exemplary bicyclic sugar moieties include LNA, ENA, cEt, S-cEt, and R-cEt.

As used herein, the term “biodistribution” refers to the cellular and/or tissue and/or organ distribution of a dsRNAi agent that is administered or delivered to a subject.

As used herein, the term “increasing”, “improving” or “changing the biodistribution properties” generally refers to the ability of a dsRNAi of the disclosure that has a greater or broader tissue distribution, as compared to the response caused by either vehicle or a control molecule/composition. Various methods known to the skilled artisan can be used to assess the increased biodistribution of the dsRNAi agents to cells or tissues, including, but not limited to, nuclear medicine, whole body autoradiography, micro-autoradiography; phosphor imaging, cryo-imaging, nano-secondary ion mass spectroscopy (nanoSIMS), matrix-assisted laser desorption imaging (MALDI-MS), radiography (X-Ray), magnetic resonance imaging (MRI), computed tomography (CT), micro-ultrasound single photon emission CT (SPECT), positron emission tomography (PET) and the like. An increased biodistribution of the dsRNAi of the disclosure, e.g., a dsRNAi of formula (I), includes an at least about 5%, at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, at least about 200%, or greater increase compared to a vehicle or a control molecule/composition. An “increased” tissue distribution or biodistribution is typically a “statistically significant” increase, and may include an increase that is 1.1, 1.2, 1.5, 2, 3, 4, 5, 6, 7, 8, 9, 10, 15, 20, 30 or more times, e.g., 500, 1000 times, (including all integers and decimal points in between and above 1, e.g., 1.5, 1.6, 1.7. 1.8, etc.) the distribution of a vehicle or a control composition. These characteristics can be important in improving the delivery and efficacy of systemically administered dsRNAi agents in the context of DM1 therapy.

“Blunt” or “blunt end” means that there are no unpaired nucleotides at that end of the double stranded RNAi agent, i.e., no nucleotide overhang. A “blunt ended” RNAi agent is a dsRNA that is double stranded over its entire length, i.e., no nucleotide overhang at either end of the molecule.

The term “complementary” means that a nucleotide, e.g., RNA or DNA, or a sequence of nucleotides are capable of base pairing non-covalently via hydrogen bonding with another nucleotide or sequence of nucleotides. As described herein and commonly known in the art the complementary (matching) nucleotide of adenosine is thymidine or uridine and the complementary (matching) nucleotide of guanosine is cytidine. The complementarity of sequences may be partial, in which only some of the nucleic acids match according to base pairing, or complete, where all the nucleic acids match according to base pairing. For example, two sequences that are complementary to each other, may have a specified percentage of nucleotides that participate in nucleobase-pairing, i.e., about 50% complementarity, preferably 50%, 55%, 65%, 70%, 75%, 80%, 85%0, 90%, 91%, 92%9, 93%, 94%, 95%, 96%, 97%, 98%, 99%, or greater complementarity over a specified region. In some embodiments, two sequences are partially complementary when the percentage of nucleotides that participate in nucleobase-pairing is about 50%, about 55%, about 65%, about 70%, about 75%, or about 80%, or ranges from about 50% to about 80%. In some embodiments, two sequences are substantially complementary when the percentage of nucleotides that participate in nucleobase-pairing is about 80%, about 81%, about 82%, about 83%, about 84%, about 85%, about 86%, about 87%, about 88%, about 89%, about 90%, about 91%, about 92%, about 92%, about 93%, about 94%, or about 95%, or ranges from about 80% to about 95%.

Examples of complementary, e.g., partially, substantially, or fully complementary, sequences are sense and antisense sequences, wherein the sense sequence contains complementary, e.g., partially, substantially, or fully complementary nucleotides to the antisense sequence and thus forms the complement of the antisense sequence. In certain aspects, a sense strand and an antisense strand of a double stranded oligonucleotide, e.g., double stranded RNA, are substantially or fully complementary over their entire lengths. In some embodiments, a sense strand and an antisense strand of a dsRNA are substantially or fully complementary over the entire length of the double stranded region of the siRNA, and one or both termini of either strand comprises single stranded nucleotides.

Further examples of complementary, e.g., partially, substantially, or fully complementary, sequences are antisense strands and their target mRNA sequence, e.g., target DMPK mRNA sequence. In certain aspects, an antisense strand is substantially or fully complementary to its target mRNA e.g., target DMPK mRNA sequence. For example, the complementary, e.g., partially, substantially, or fully complementary, sequences may be between an antisense strand and a coding region of the target mRNA, e.g., DMPK mRNA, or a non-coding sequence of the target mRNA. In certain aspects, an antisense strand is substantially, or fully complementary to its target mRNA, e.g., DMPK mRNA, to reduce or eliminate off-target profile for and to improve down-regulation of the target gene, e.g., gene of the target mRNA sequence.

The term “control” or “control experiment” is used in accordance with its plain ordinary meaning and refers to an experiment in which the subjects or reagents of the experiment are treated as in a parallel experiment except for omission of a procedure, reagent, or variable of the experiment. Typically, a control is used as a standard of comparison in evaluating experimental effects. In some embodiments, a control is the measurement of the expression of a protein or mRNA, e.g., DMPK mRNA, in the absence of RNAi agents as described herein.

“Delay the onset of” means to delay the development of a condition or disease in a subject who is at risk for developing the disease or condition. In some embodiments, a subject at risk for developing a disease or condition is identified using clinical assessments similar to those used to diagnose the disease or condition. For example, a subject at risk for developing DM1 may be identified by genetic testing for amplification of the CTG repeat in the DMPK gene. In some embodiments, a subject at risk for developing the disease or condition receives treatment similar to the treatment received by a subject who already has the disease or condition.

As used herein the phrase “disease-relevant tissue” means a tissue that displays biochemical or histological pathology associated with myotonic dystrophy type 1. Exemplary disease-relevant tissue includes that from heart, diaphragm, quadriceps, gastrocnemius, soleus, brain.

“Double stranded nucleic acid” means a first nucleotide sequence and a second nucleotide sequence can form a double stranded nucleic acid or duplex structure by complementation. Double stranded nucleic acids include structures formed from annealing a first oligonucleotide to a second, complementary oligonucleotide, as in an siRNA. Such double stranded nucleic acids may have a short nucleotide overhang at one or both ends of the duplex structure. Double stranded nucleic acids also include structures formed from a single oligonucleotide with sufficient length and self-complementarity to form a duplex structure, as in an shRNA. Such double-stranded nucleic acids include stem-loop structures. A double-stranded nucleic acid may include one or more modifications relative to a naturally occurring terminus, sugar, nucleobase, and/or phosphate group.

As used herein, the terms “double stranded region” and “duplex region” are used interchangeably to refer to the structure formed by the complementary pairing between two regions of a RNA molecule. A double stranded region may comprise a Watson-Crick base pair, a non-Watson-Crick base pair or a combination thereof. In certain aspects, the “double stranded region” or “duplex region” contains a portion of a double stranded nucleic acid wherein nucleotides are capable of hydrogen bonding to each other. Such hydrogen bonding can be intramolecular or intermolecular, e.g., single transcription unit forming a double stranded region with the so-called hairpin or two transcription units that align appropriately for complementary sequences to hydrogen bond. To be a double stranded region, it is not necessary for 100% of the nucleotides to be complementary and hydrogen bonded within a region. It is merely necessary for sufficient base pairing to occur to give the RNA a substantial double stranded character, e.g. an indicative melting point. A double stranded region can be a defined portion within a double stranded nucleic acid that is shorter than, e.g. encompassed by, the full double stranded nucleic acid. Alternatively, a double stranded region can be the same length as the full double stranded nucleic acid. A double stranded region may contain one or more mismatches between the first and second nucleotide sequence, and retain the ability to hybridize with each other. Double stranded regions do not include nucleotide overhangs.

“ENA sugar” means a substituted sugar moiety comprising a —(CH₂)₂—O-linkage between the 4′ and 2′ furanose ring atoms.

As used herein, the term “fatty acid moiety” means a structure incorporating in part, a fatty acid or is derived from a fatty acid as a building block.

The term “fatty acid” means a carboxylic acid having the formula RCOOH. R represents an aliphatic group, preferably an alkyl or alkenyl group. R can comprise between about 1 and about 50 carbon atoms. Fatty acids can be saturated, monounsaturated, or polyunsaturated.

As used herein, the term “having suppressing activity of DMPK gene expression” can be used interchangeably with “inhibiting or decreasing DMPK gene expression” and “inhibiting expression of DMPK”, and refers to the at least partial suppression of the expression of a DMPK gene, as manifested by a reduction of the amount of DMPK mRNA, which may be isolated from or detected in a group of cells (“a first cell”) in which a DMPK gene is transcribed and which has or have been treated such that the expression of a DMPK gene is inhibited, as compared to group of cells substantially identical to the first cell but without treated (“control cells” or “a second cell”). For example, partial suppression of the expression of a DMPK gene refers to a decrease in DMPK gene expression by about 10%, about 20%, about 30%, about 40%, about 50%, about 60%, about 70%, about 80%, about 90% or greater in comparison to a control, in particular, when measured by RT-PCR, e.g., RT-qPCR.

In some embodiments, the level or expression of the DMPK gene can be measured by evaluation of mRNA, e.g., via Northern blots or PCR. The effect of an RNAi agent on the DMPK gene expression can be determined by measuring the gene transcription rates, e.g., via Northern blots; or reverse transcriptase polymerase chain reaction or real-time polymerase chain reaction). In some embodiments, the degree of inhibition can be calculated as the following equation:

$$\frac{( mRNAin c ontrolcells ) - ( mRNAin treatedcells )}{( mRNAin controlcells )} \cdot 100 \%$$

As used herein, the term “hybridize” means the annealing of one nucleotide sequence to another nucleotide sequence based at least in part on nucleotide sequence complementarity. In some embodiments, an antisense strand is hybridized to a sense strand. In some embodiments, an antisense strand hybridizes to a target mRNA sequence.

The terms “identical” or “percent identity” in the context of two or more nucleic acids, refer to two or more sequences or subsequences that are the same or have a specified percentage of nucleotides that are the same, i.e., at least 60% identity, or at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or within a range defined by any of two of the preceding values, identity over a specified region when compared and aligned for maximum correspondence over a comparison window or designated region, as measured using a BLAST or BLAST 2.0 sequence comparison algorithms with default parameters described below, or by manual alignment and visual inspection, see, e.g., NCBI web site or the like. This definition also refers to, or may be applied to, the complement of a test sequence. The definition also includes sequences that have deletions and/or additions, as well as those that have substitutions. As described below, the preferred algorithms can account for gaps, insertions and the like. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “inhibitor” also refers to a compound, composition, or substance capable of detectably negatively affecting, e.g., decreasing activity, expression or function of a given protein or gene, e.g., a DMPK gene. For example, an inhibitor may decrease activity, expression or function by about 10%, about 20%, about 30%, about 40%, about 50%, about 60%, about 70%, about 80%, about 90% or greater in comparison to a control in the absence of the inhibitor. Inhibitors include, for example, synthetic or biological molecules, such as oligonucleotides. In some embodiments, the inhibitors include a RNAi agent, e.g., siRNA agent, dsRNAi agent, or dsRNA agent.

The term “ligand”, as used herein, refers to a compound or moiety that can impose characteristics to provide additional properties, e.g., affinity or cell delivery efficiency, to an RNAi, e.g., dsRNAi, as described herein. The ligand may be coupled or conjugated directly to the RNAi, e.g., sense strand or antisense strand of dsRNA, or indirectly to the RNAi agent, e.g., sense strand or antisense strand of dsRNA, via an intervening linker (“linker”). When a ligand is conjugated or coupled indirectly to the RNAi, e.g., dsRNA via a linker, the ligand may be formed of a core moiety, e.g., targeting moiety that has specific function to provide affinity or efficacy and the linker that provides merely an optimal distance, e.g., between the core moiety and the RNAi agent (dsRNA). In certain aspects, the term “ligand” embraces the ligand in combination with the linker. Examples of ligands or targeting moieties thereof may include, but not be limited to, one or more selected from a synthetic or natural compound, a fatty acid, e.g., long and medium chain fatty acids, a peptide, an antibody, a carbohydrate, e.g., sugar moiety, or an additional nucleic acid. In embodiments, the ligand as disclosed herein, is a fatty acid or fatty acid moiety in combination with a linker.

The term “linker”, as used herein, refers to a molecule that is used to join an oligonucleotide to a fatty acid moiety as disclosed herein. The linker is capable of forming covalent bonds to both the oligonucleotide and to the fatty acid moiety. In an embodiment, the linker is a bifunctional linker. The term “bifunctional linker”, as used herein, refers to linkers which contain at least two functional groups for the purpose of coupling an oligonucleotide to a fatty acid moiety. One functional group is used, for example, to form a covalent bond between the oligonucleotide and the linker. The second functional group is used, for example, to form a covalent bond between the linker and the fatty acid moiety. Suitable linkers are well known to those of skill in the art and include, but are not limited to, straight or branched-chain carbon linkers, heterocyclic carbon linkers, amino linkers, or peptide linkers. The procedure for conjugating two molecules varies according to the chemical structure of the agent. The linker, as disclosed herein, typically contains one or more functional groups, e.g., carboxylic acid (—COOH), hydroxyl (—OH), thiol (—SH), or free amine (—NH₂) groups, which are available for reaction with a suitable functional group on the oligonucleotide, or on the fatty acid moiety, to join the molecules thereto.

“Locked nucleic acid (LNA) sugar” means a substituted sugar moiety comprising a —CH₂—O— linkage between the 4′ and 2′ furanose ring atoms.

A “mismatch” is defined herein as a difference between the base sequence, e.g., U instead of A, or length when two sequences are maximally aligned and compared. In certain aspects, the term “mismatch” means a nucleobase of a first oligonucleotide, e.g., a first strand, that is not capable of pairing with a nucleobase at a corresponding position of a second oligonucleotide, e.g., a second strand.

A “modified internucleoside linkage” means any change from a naturally occurring phosphodiester linkage between two nucleotides.

A “modified nucleobase” means a nucleobase having one or more alterations relative to a naturally occurring nucleobase.

The term “modified nucleotide” means a nucleotide having one or more modifications relative to a naturally occurring nucleotide, e.g., RNA. The modified nucleotide may be selected over an unmodified form because of desirable properties such as, for example, enhanced cellular uptake, enhanced affinity for other oligonucleotides or nucleic acid targets, increased stability in the presence of nucleases, and/or reduced immune stimulation. In certain aspects, the modification may be present in at least one of (i) an internucleoside linkage (“linkage”), (ii) a nucleobase, and (iii) a sugar moiety of the nucleotide. In certain aspects, the modification is present in the internucleoside linkage, e.g., by chemically modifying a phosphate (or phosphodiester) linkage or replacing a phosphate (or phosphodiester) linkage with other linking groups. In certain aspects, the modification is present in a sugar moiety, i.e., ribose ring, by substituting hydroxyl group on 2′ position of the ribose ring with other chemical group or by replacing a ring structure with other heterocycloalkyl or cycloalkyl, glycol group having a structure of

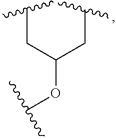 [SMILES: *CC(C*)O*]

bicyclic or bridged ring on the ribose such as locked nucleic acid (LNA) having a structure of

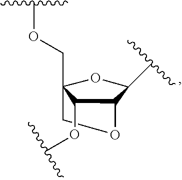 [SMILES: *OC[C@@]12COC([C@H](*)O1)[C@H]2O*]

or the like. In certain aspects, the modification is present in a nucleobase (e.g., A, G, C, T, or U) by chemical modification in a nucleobase by replacing the nucleobase with other moiety, for example, by replacing one naturally occurring nucleobase with another naturally occurring nucleobase. In certain aspects, a modified nucleotide may contain a modification in a sugar moiety and an unmodified phosphate (or phosphodiester) linkage. In certain aspects, a modified nucleotide may have a modification in a sugar moiety but with an unmodified nucleobase. In certain aspects, a modified nucleotide may have a modification in a sugar moiety and a nucleobase. In certain aspects, a modified nucleotide may have a modification in a sugar moiety and a phosphate (or phosphodiester) linkage. In certain aspects, a modified nucleotide may have a modification in a sugar moiety, a phosphate (or phosphodiester) linkage and a nucleobase. In certain aspects, a modified nucleotide may have an unmodified sugar moiety and an unmodified phosphate (or phosphodiester) linkage. In certain aspects, a modified nucleotide may have an unmodified sugar moiety and an unmodified nucleobase. In certain aspects, a modified nucleotide may have an unmodified sugar moiety and a modified nucleobase. In certain aspects, a modified nucleotide may have an unmodified sugar moiety and a modified phosphate (or phosphodiester) linkage. In certain aspects, a modified nucleotide may have a modified sugar moiety, a modified phosphate (or phosphodiester) linkage and a modified nucleobase.

The term “modified phosphate group” or “modified phosphodiester linkage” as used herein refers to a chemical group in place of a phosphate group (or phosphodiester linkage) in a nucleotide as being attached to the 3′ end (3′ carbon) of the pentofuranosyl group.

A “modified sugar moiety” means a sugar of a nucleotide having any change and/or substitution from a naturally occurring sugar moiety.

“Myotonic dystrophy type 1” or “DM1” means an inherited multisystem disorder caused by abnormal expansion of a CTG repeat in the 3′-untranslated region of the DM1 protein kinase (DMPK) gene, and characterized by progressive muscle atrophy, progressive muscle weakness, and myotonia.

As used herein, the term “multibranched’ refers to a non-linear structure. A multibranched structure may have one or more side chains or branches extending from a main backbone. The backbone may contain one or more functional groups capable of forming covalent bonds with other molecular groups. In an embodiment, the side chains or branches include fatty acids or a part thereof. For example, the backbone may contain primary or secondary amine groups that form amide linkages with the carboxylic acid groups of fatty acids.

The term “nucleic acid” means a compound containing at least two nucleotide monomers covalently linked together. Nucleic acids include polynucleotides and oligonucleotides, including double-stranded oligonucleotides and single-stranded oligonucleotides, and modified versions thereof. Double stranded nucleic acid means that the first oligonucleotide strand and the second oligonucleotide strand can form a double stranded nucleic acid structure or duplex by complementation.

The term “nucleobase” or “base”, as used herein, means the heterocyclic base moiety of a nucleoside or nucleotide. Non-limiting examples of nucleobases includes cytosine or a derivative thereof, e.g., cytosine analogue, guanine or a derivative thereof, e.g., guanine analogue, adenine or a derivative thereof, e.g., adenine analogue, thymine or a derivative thereof, e.g., thymine analogue, uracil or a derivative thereof, e.g., uracil analogue, hypoxanthine or a derivative thereof, e.g., hypoxanthine analogue, xanthine or a derivative thereof, e.g., xanthine analogue, 7-methylguanine or a derivative thereof, e.g., 7-methylguanine analogue, deaza-adenine or a derivative thereof, e.g., deaza-adenine analogue, deaza-guanine or a derivative thereof, e.g., deaza-guanine, deaza-hypoxanthine or a derivative thereof, 5,6-dihydrouracil or a derivative thereof, e.g., 5,6-dihydrouracil analogue, 5-methylcytosine or a derivative thereof, e.g., 5-methylcytosine analogue, or 5-hydroxymethylcytosine or a derivative thereof, e.g., 5-hydroxymethylcytosine analogue, moieties. In some embodiments, the nucleobase is adenine, guanine, hypoxanthine, xanthine, theobromine, caffeine, uric acid, or isoguanine, which may be optionally substituted or modified. In some embodiments, the nucleobase is

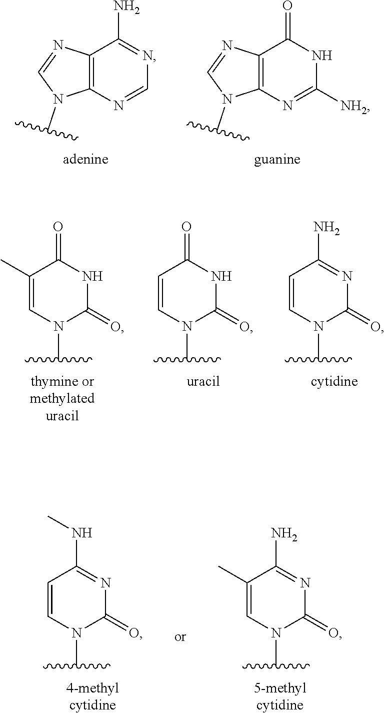

which may be optionally substituted or modified, wherein “ [symbol] ” denotes the point of attachment to a pentofuranosyl sugar ring, e.g., 1′ position.

The term “nucleoside” means a monomer consisting of a nucleobase and a pentofuranosyl sugar, e.g., ribose or deoxyribose. A nucleoside including a ribose sugar ring has to a structure of

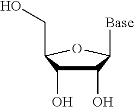

or a pharmaceutically acceptable salt thereof, and a nucleoside including a deoxyribose sugar ring has a structure of

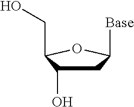

or a pharmaceutically acceptable salt, wherein in each structure, “Base” is a nucleobase.

The term “nucleotide” means a compound including a nucleoside and a phosphate group (or phosphodiester linkage) that are covalently attached at 5′ position or 3′ position of the pentofuranosyl sugar, e.g., ribose or deoxyribose. In certain aspects, the nucleotide is a ribonucleotide (RNA) having the ribose as the pentofuranosyl sugar. In certain aspects, a nucleotide is a deoxyribonucleotide (DNA) having the deoxyribose (2′-deoxyribose) as the pentofuranosyl sugar. Unless otherwise specifically indicated, when referring a “nucleotide” in a chain of nucleotides, e.g., oligonucleotides, a nucleotide is meant by a nucleoside and a phosphate group (or phosphodiester linkage) that is covalently attached at 3′ position of the pentofuranosyl sugar, e.g., ribose or deoxyribose.

The term “oligonucleotide” means a shorter length nucleic acid, e.g., of less than 100 nucleotides in length. Oligonucleotides may be single-stranded or double-stranded. In some embodiments, an oligonucleotide may include naturally occurring ribonucleotides, naturally occurring deoxyribonucleotides, and/or nucleotides having one or more modifications to a naturally occurring terminus, sugar, nucleobase, and/or internucleoside linkage. Non-limiting examples of oligonucleotides include double-stranded oligonucleotides, e.g., dsRNA, single-stranded oligonucleotides, e.g., single stranded RNA or ssRNA, antisense oligonucleotides (“ASO”), small interfering RNA (siRNA), microRNA mimics, short hairpin RNAs (shRNA), single-strand small interfering RNA (ssRNAi), RNaseH oligonucleotides, anti-microRNA oligonucleotides, steric blocking oligonucleotides, exon-skipping oligonucleotides, CRISPR guide RNAs, and aptamers. In certain aspects, the oligonucleotide is a dsRNA and each strand has a length less than 100 nucleotides (“nt”), less than 90 nt, less than 80 nt, less than 70 nt, less than 60 nt, less than 50 nt, less than 40 nt, less than 35 nt, less than 30 nt, less than 28 nt, less than 26 nt, less than 25 nt, less than 24 nt, less than 23 nt, less than 22 nt, less than 21 nt, less than 20 nt, less than 19 nt, less than 18 nt, less than 17 nt, less than 16 nt, or 15 nt.

The term “overhang” or “nucleotide overhang” herein refers to at least one unpaired nucleotide that protrudes from the end of at least one of the two strands of the duplex structure of an RNAi agent. In some embodiments, when a 3′-end of one strand extends beyond the 5′-end of the other strand, or vice versa, this forms a nucleotide overhang, e.g., the unpaired nucleotide(s) form the overhang.

The term “phosphate,” or “phosphate group” as used herein means a chemical species made of one phosphorus atom and four oxygen atoms

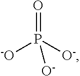

or esters, salts, or acids thereof. In certain aspects, when the phosphate groups are positioned between adjacent nucleosides in an RNA or DNA strand and form a “backbone” of the oligonucleotides, these terms “phosphate,” or “phosphate group” may be interchangeably used as “phosphate group,” “phosphate linkage,” “phosphodiester linkage,” or “linkage.” For example, the phosphate or phosphodiester linkage in the backbone of RNA or DNA may have the structures of

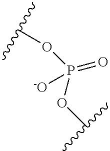

or esters, salts, e.g., pharmaceutically acceptable salts, or acids, e.g.

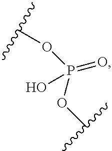 [SMILES: *OP(=O)(O)O*]

thereof, wherein “ [symbol] ” denoted the point of attachment to pentofuranosyl sugar rings, e.g., 5′ and 3′ positions) in adjacent nucleosides. In certain aspects, a variant of a phosphate or phosphodiester linkage, e.g., phosphorothioate (PS) linkage, can replace a phosphate group (or phosphodiester linkage) in the backbone and connect two adjacent nucleosides. In certain aspects, a variant of a phosphate or phosphodiester linkage, e.g., phosphorothioate (PS) linkage or vinyl phosphonate (VP) group, may be additionally attached at 3′ end or 5′ end of the oligonucleotides, e.g., RNA or DNA, e.g., 3′-OH or 5′-OH position of the terminal pentofuranosyl sugar, e.g., ribose or deoxyribose, so as to act as chemically or biologically functional group. In certain aspects, a variant of phosphate or phosphodiester linkage may also be referred as a phosphorus derived internucleoside linkage that includes at least one phosphorus atom in the backbone.

Unless otherwise indicated herein, an unmodified RNA (or “ribonucleotide”) in a chain of nucleotides, e.g., sense strand or antisense strand of siRNA, as disclosed refers to a structure of

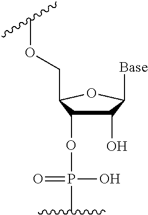

or a pharmaceutically acceptable salt thereof. In the above structure “Base” is a nucleobase and [symbol] is an attachment point to the adjacent nucleotides.

Unless otherwise indicated herein, when an unmodified RNA is the first nucleotide from the 5′ end of an RNA chain, e.g., sense strand or antisense strand of siRNA, that nucleotide has a structure of

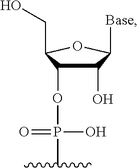

or a pharmaceutically acceptable salt thereof. In the above structure “Base” is a nucleobase and [symbol] is an attachment point (5′ oxygen) to the adjacent nucleotides.

Unless otherwise indicated herein, when an unmodified RNA is the first nucleotide from the 3′ end of an RNA chain, e.g., sense strand or antisense strand of siRNA, that nucleotide has a structure of

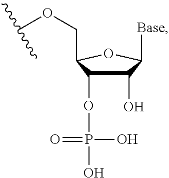

or a pharmaceutically acceptable salt. In certain embodiments, when an unmodified RNA is the first nucleotide from the 3′ end of an RNA chain, e.g., sense strand or antisense strand of siRNA, that nucleotide does not include 3′ end phosphate group or phosphodiester linkage, for example, which has been removed during hydrolysis or synthesis, has a structure of

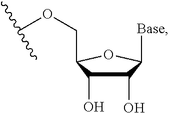

or a pharmaceutically acceptable salt. In the above structure “Base” is a nucleobase and [symbol] is an attachment point, e.g., phosphorus of the phosphate linkage, to the adjacent nucleotides.

A code “A”, “G”, “C”, “U”, or “T” presented in a sequence list as disclosed herein stand for a RNA nucleotide that contains adenine, guanine, cytosine, uracil and thymine as a base, respectively.

As used herein, the term “pharmaceutically acceptable salt” means a salt form of a compound that retains the biological effectiveness and properties of a compound and does not have undesired effects when administered to a subject. In many cases, the compounds of the present disclosure are capable of forming acid and/or base salts by virtue of the presence of, for example, hydroxyl, amino and/or carboxyl groups or groups similar thereto. When both a basic group and an acid group are present in the same molecule, the compounds of the present disclosure may also form internal salts, e.g., zwitterionic molecules. In certain aspect, pharmaceutically acceptable acid addition salts can be formed with inorganic acids and organic acids. Examples of the inorganic acids from which salts can be derived include, for example, hydrochloric acid, hydrobromic acid, sulfuric acid, nitric acid, phosphoric acid, and the like. Examples of the organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. In certain aspect, the pharmaceutically acceptable base addition salts can be formed with inorganic and organic bases. Examples of the inorganic bases from which salts can be derived include, for example, ammonium salts and metals from columns I to XII of the periodic table, such as sodium, potassium, ammonium, calcium, magnesium, iron, silver, zinc, and copper; particularly suitable salts include ammonium, potassium, sodium, calcium and magnesium salts. Examples of the organic bases from which salts can be derived include, for example, primary, secondary, and tertiary amines, substituted amines including naturally occurring substituted amines, cyclic amines, basic ion exchange resins, and the like, such as organic amines include isopropylamine, benzathine, cholinate, diethanolamine, diethylamine, lysine, meglumine, piperazine and tromethamine.

In certain aspects, the term “pharmaceutically acceptable salt” as used herein may include the salts forms in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate.

For dsRNAi agents of the present disclosure, particular examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

A “phosphorothioate internucleoside linkage” means a substituted phosphodiester internucleoside linkage where one of the non-bridging atoms is a sulfur atom.

As used herein, the term “prevent”, “preventing” or “prevention” of any disease or disorder refers to the prophylactic treatment of the disease or disorder; or delaying the onset or progression of the disease or disorder.

The terms “iRNA”, “RNAi agent”, “iRNA agent”, “RNA interference agent” as used interchangeably herein, refer to an agent that contains RNA as that term is defined herein, and which mediates the targeted cleavage of an RNA transcript (mRNA) via an RNA-induced silencing complex (RISC) pathway. An RNAi agent directs the sequence-specific degradation of mRNA through a process and thereafter inhibits expression of the gene encoded by the mRNA in a cell in vivo, e.g., in a subject, e.g., any vertebrate, mammal, or human.

As used herein, the terms “salt” or “salts” refers to an acid addition or base addition salt of a compound of the present disclosure. “Salts” include in particular “pharmaceutical acceptable salts”.

The term “sense strand,” as used herein, refers an oligonucleotide that is complementary, e.g., partially, substantially, or fully complementary, to the antisense strand. The sense strand is typically degraded following incorporation of the antisense strand into RISC. The sense strand may also be referred to as the “passenger strand”. In some embodiments, the sense strand may have a length from 15-30 nt, 15-26 nt, 15-23 nt, 15-22 nt, 15-21 nt, 15-20 nt, 15-19 nt, 15-18 nt, 15-17 nt, 18-30 nt, 18-26 nt, 18-23 nt, 18-22 nt, 18-21 nt, 18-20 nt, 19-30 nt, 19-26 nt, 19-23 nt, 19-22 nt, 19-21 nt, 19-20 nt, 19 nt, 20-30 nt, 20-26 nt, 20-25 nt, 20-24 nt, 20-23 nt, 20-22 nt, 20-21 nt, 20 nt, 21-30 nt, 21-26 nt, 21-25 nt, 21-24 nt, 21-23 nt, 21-22 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, or 36 nt.

The term “small interfering RNA” or “siRNA” means a double stranded nucleic acid (dsRNA) formed with two anti-parallel, and partially, substantially or fully complementary nucleic acid strands, e.g., a first strand and a second strand; or a “sense” strand and an “antisense” strand, which interferes with the expression of genes in a sequence-specific manner by facilitating mRNA degradation before translation through the RNA interference pathway. In some embodiments, depending on the context, the first strand can be a “guide” or antisense strand, and the second strand can be a “passenger” or sense strand. The antisense and sense strands of an siRNA are not covalently linked. In some embodiments, depending on the context, the “first” strand can be a passenger or sense strand, and the “second” strand can be a guide or antisense. In certain aspects, an “RNAi agent” or “siRNA agent,” as used herein, refers a double stranded RNA (dsRNA) with or without a ligand or other conjugate, and may be interchangeably used with a term “double stranded RNAi agent (dsRNAi agent),” or “dsRNA agent.” In certain aspect of the disclosure, the term “siRNA” can be used to describe a dsRNA with specific nucleotide sequences (unmodified or modified nucleotide sequences), without a ligand or other conjugate.

“shRNA” means a double stranded nucleic acid containing a loop structure that is processed in a cell to an siRNA which directs gene silencing in a sequence-specific manner, by facilitating mRNA degradation before translation through the RNA interference pathway.

The terms “subject” and “patient” as used herein are used interchangeably. The term subject includes a human or non-human animal, preferably a vertebrate, and more preferably a mammal. In certain aspects, the subject is a human. In certain aspects, the subject is a human patient.

As used herein, a subject is “in need of” a treatment if such subject would benefit biologically, medically or in quality of life from such treatment.

The term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, “target sequence” or “target gene” refer to a contiguous portion of the nucleotide sequence of an mRNA molecule formed during the transcription of a gene including mRNA that is a product of RNA processing of a primary transcription product. The target portion of the sequence will be at least long enough to serve as a substrate for RNAi-directed cleavage at or near that portion. For example, the target sequence will generally be from 9-36 nucleotides (“nt”) in length, e.g., 15-30 nt in length, including all sub-ranges therebetween. As non-limiting examples, the target sequence may have a length from 15-30 nt, 15-26 nt, 15-23 nt, 15-22 nt, 15-21 nt, 15-20 nt, 15-19 nt, 15-18 nt, 15-17 nt, 18-30 nt, 18-26 nt, 18-23 nt, 18-22 nt, 18-21 nt, 18-20 nt, 19-30 nt, 19-26 nt, 19-23 nt, 19-22 nt, 19-21 nt, 19-20 nt, 19 nt, 20-30 nt, 20-26 nt, 20-25 nt, 20-24 nt, 20-23 nt, 20-22 nt, 20-21 nt, 20 nt, 21-30 nt, 21-26 nt, 21-25 nt, 21-24 nt, 21-23 nt, 21-22 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, or 36 nt.

The term “effective amount” or “therapeutically effective amount’ of a compound, e.g., siRNA, as disclosed herein refers to an amount of the compound that will elicit the biological or medical response of a subject, or ameliorate symptoms, alleviate conditions, slow or delay disease progression, or prevent a disease, etc. In certain aspects, the term “a therapeutically effective amount” refers to the amount of the compound, e.g., siRNA, of the disclosure that, when administered to a subject, is effective to produce a desired therapeutic effect, for example, that is sufficient to significantly reduce or inhibit the expression of DMPK mRNA in the target cells or tissues, thereby mitigating or alleviating one or more symptoms of myotonic dystrophy type 1 (DM1) in a subject. This may include reducing the sequestration of muscleblind-like (MBNL) proteins and other RNA-binding proteins that are aberrantly bound by the toxic DMPK mRNA. By alleviating this sequestration, the siRNA treatment can restore normal cellular function and improve muscle function and other physiological processes affected by the disease. The specific amount can vary depending on factors such as the delivery method, the severity of the disease, the genetic profile of the subject, and the particular siRNA sequence used. An effective amount or therapeutically effective amount is typically determined by empirical studies and may be optimized to achieve the desired therapeutic effect while minimizing potential side effects. In certain aspects, the effective amount or therapeutically effective amount of a compound administered to the subject treats, ameliorates, delays or reduces a symptom of DM1 in a subject.

For any compound described herein, the therapeutically effective amount can be initially determined from cell culture assays. Target concentrations will be those concentrations of active compound(s) that are capable of achieving the methods described herein, as measured using the methods described herein or known in the art. Therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. An example of an “therapeutically effective amount” is an amount sufficient to contribute to the treatment, prevention, or reduction of a symptom or symptoms of a disease. For example, for the given parameter, e.g., biomarker, a therapeutically effective amount will show an increase or decrease of at least about 5%, at least about 10%, at least about 15%, at least about 20%, at least about 25%, at least about 40%, at least about 50%, at least about 60%, at least about 75%, at least about 80%, at least about 90%, or at least about 95%. Therapeutic efficacy can also be expressed as “-fold” increase or decrease. For example, a therapeutically effective amount can have at least a 1.2-fold, 1.5-fold, 2-fold, 5-fold, or more effect over a control.

The term “therapy”, as used herein refers to an application of one or more specific procedures used for the amelioration of at least one indicator or a disease or condition. In certain aspect, the specific procedure is the administration of one or more pharmaceutical or therapeutic agents.

As used herein, the term “treat”, “treating”, or “treatment” of any disease or disorder refers to alleviating or ameliorating the disease or disorder, i.e., slowing or arresting the development of the disease or at least one of the clinical symptoms thereof; or alleviating or ameliorating at least one physical parameter or biomarker associated with the disease or disorder, including those which may not be discernible to the patient. In some embodiments, treating does not include preventing.

The term “unsubstituted” means that the specified group bears no substituents.

“2′-O-methyl sugar” or “2′-OMe sugar” means a sugar having an —O—CH₃ substitution at the 2′ position of the pentofuranosyl sugar.

“2′-O-methoxyethyl sugar” or “2′-MOE sugar” means a sugar having an —OCH₂CH₂OCH₃ substitution at the 2′ position of the pentofuranosyl sugar.

“2′-fluoro sugar” or “2′-F sugar” means a sugar having a fluoro substitution at the 2′ position of the pentofuranosyl sugar.

“2′-O-methyl nucleotide” means a nucleotide having an —O-methyl substitution at the 2′ position of the pentofuranosyl sugar. A 2′-O-methyl nucleotide may have a further modification in addition to the modified sugar moiety, for example a modified nucleobase and/or phosphate group.

“2′-fluoro nucleotide” means a nucleotide having a fluoro substitution at the 2′ position of the pentofuranosyl sugar. A 2′-O-fluoro nucleotide may have a further modification in addition to the modified sugar moiety, for example a modified nucleobase and/or phosphate group.

“5′-(E)-vinylphosphonate” or “5′-VP”, refers to a chemical moiety having the structure:

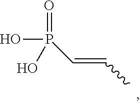

or salts thereof, wherein the wavy line represents the point of attachment to the 5′ carbon of the pentafuranosyl sugar of a nucleotide.

Unless defined otherwise, the chemical structures and formulae set forth herein are constructed according to the standard rules of chemical valency known in the chemical arts.

The term “alkyl” means a straight or branched chain saturated hydrocarbon containing 1-30 carbon atoms. The alkyl may include a designated number of carbons, e.g., C₁-C₁₀ means one to ten carbons. Examples of alkyl include, but are not limited to, groups such as C₁-C₃₀ alkyl, C₁-C₂₅ alkyl, C₁-C₂₀ alkyl, C₁-C₁₅ alkyl, C₁-C₁₂ alkyl, C₁-C₁₀ alkyl, C₁-C₈ alkyl, C₁-C₆ alkyl, C₁-C₄ alkyl, or C₁-C₃ alkyl. For example, C₁-C₆ alkyl include, but is not limited to, methyl, ethyl, n-propyl, 1-methylethyl (iso-propyl), n-butyl, n-pentyl and 1,1-dimethylethyl (t-butyl), and their isomers.

The term “alkylene”, means, unless otherwise stated, a divalent radical derived from an alkyl, as defined herein. Examples of alkylene include, without limitation, —(CH₂)ₙ—, wherein n is the number of CH₂ repeat units of the alkylene chain.

The term “alkylthio”, as used herein, means an alkyl group, as defined herein, appended to the parent molecular moiety through a sulfur atom. Representative examples of alkylthio include, but are not limited, methylthio, ethylthio, tert-butylthio, and hexylthio.

“Alkenyl” means a straight or branched chain unsaturated hydrocarbon containing 2-30 carbon atoms. The “alkenyl” group contains at least one double bond in the chain. The double bond of an alkenyl group can be unconjugated or conjugated to another unsaturated group. Like the alkyl, the alkenyl may include a designated number of carbons, e.g., C₂-C₂₀ means two to twenty carbons. Examples of alkenyl include, but are not limited to, groups such as C₂-C₃₀ alkenyl, C₂-C₂₅ alkenyl, C₂-C₂₀ alkenyl, C₂-C₁₅ alkenyl, C₂-C₁₂ alkenyl, C₂-C₁₀ alkenyl, C₂-C₈ alkenyl, C₂-C₆ alkenyl, C₂-C₄ alkenyl, or C₂-C₃ alkenyl. Examples of alkenyl include, without limitations, ethenyl (or vinyl), propenyl, e.g., prop-1-enyl, prop-2-enyl, butadienyl, e.g., buta-1,3-dienyl, butenyl, e.g., but-1-en-1-yl, but-2-en-1-yl but-3-en-1-yl, pentenyl, e.g., pent-1-en-1-yl, pent-2-en-2-yl, hexenyl, e.g., hex-1-en-2-yl, hex-2-en-1-yl, 1-ethylprop-2-enyl, 1,1-(dimethyl)prop-2-enyl, 1-ethylbut-3-enyl, and 1,1-(dimethyl)but-2-enyl.

“Alkenylene” means, unless otherwise stated, a divalent radical derived from an alkenyl, as defined herein.

As used herein, the term “alkoxy” refers to a radical of the formula —OR^{a} where Ra is an alkyl, e.g., C₁₋₃₀ alkyl, C₁₋₂₅ alkyl, C₁₋₂₀ alkyl, C₁₋₁₅ alkyl, C₁₋₁₂ alkyl, C₁₋₁₀ alkyl, C₁₋₈ alkyl, C₁₋₆ alkyl, C₁₋₄ alkyl, or C₁₋₃ alkyl, radical as generally defined above. For example, C₁₋₈ alkoxy includes, but is not limited to, methoxy, ethoxy, propoxy, isopropoxy, butoxy, isobutoxy, pentoxy, and hexoxy.

“Alkynyl” means a straight or branched chain unsaturated hydrocarbon containing 2-30 carbon atoms. The “alkynyl” group contains at least one triple bond in the chain. Examples of alkynyl include, but are not limited to C₂-C₂₀, C₂-C₁₂, C₂-C₈, C₂-C₆, or C₂-C₄ alkynyl. The term “C₂-C₈ alkynyl” is to be construed accordingly. Examples of alkynyl groups include, without limitation, ethynyl, propargyl, n-butynyl, isobutynyl, pentynyl, and hexynyl. An alkynyl group can be unsubstituted or substituted. Examples of “C₂-C₆alkynyl” include, without limitations, ethynyl, prop-1-ynyl, prop-2-ynyl, and but-2-ynyl.

“Alkynylene” means, unless otherwise stated, a divalent radical derived from an alkynyl, as defined herein.

The term “cycloalkyl” means a monocyclic, bicyclic, or a polycyclic saturated or unsaturated carbon ring containing 3-18 carbon atoms wherein there are no delocalized pi electrons (aromaticity) shared among the ring carbon. The terms “C₃-C₁₀cycloalkyl”, “C₃-C₈cycloalkyl” and “C₃-C₆cycloalkyl” are to be construed accordingly. The term polycyclic encompasses bridged, e.g., norborane, fused, e.g., decalin, and spirocyclic cycloalkyl. In embodiments, cycloalkyl, e.g., “C₃-C₁₀ cycloalkyl”, is a monocyclic hydrocarbon group of 3 to 10 carbon atoms, respectively. In some embodiments, cycloalkyl groups are fully saturated.

Examples of cycloalkyl groups include, without limitations, cyclopropenyl, cyclopropyl cyclobutyl, cyclobutenyl, cyclopentyl, cyclohexyl, cycloheptanyl, cyclooctanyl, norboranyl, norborenyl, spiro[2.3]hexanyl, spiro[3.3]heptyl, spiro[3.4]octanyl, spiro[2.5]octanyl, spiro[3.5]nonanyl, spiro[4.5]decanyl, spiro[5.5]undecanyl, spiro[4.4]nonanyl, bicyclo[2.2.2]octanyl, bicyclo[2.2.2]octenyl, bicyclo[1.1.1]pentanyl and derivatives thereof.

Examples of C₃-C₁₀ cycloalkyl include, without limitations, cyclopropyl, cyclobutyl, cyclopentyl, cyclohexyl, spiro[2.3]hexanyl, spiro[3.3]heptyl, spiro[3.4]octanyl, spiro[2.5]octanyl, spiro[3.5]nonanyl, spiro[4.5]decanyl, spiro[5.5]undecanyl, spiro[4.4]nonanyl, bicyclo[1.1.1]pentyl, bicyclo[2.1.1]hexyl, bicyclo[2.1.1]heptyl, bicyclo[2.2.2]octyl, and bicyclo[1.1.1]pentanyl.

Examples of C₃-C₈cycloalkyl include, without limitations, cyclopropyl, cyclobutyl, cyclopentyl, cyclohexyl, cycloheptyl, spiro[3.3]heptanyl, and cyclooctyl.

Examples of C₃-C₆cycloalkyl include, without limitations, cyclopropyl, cyclobutyl, cyclopentyl, and cyclohexyl.

In some embodiments, cycloalkyls include, but are not limited to, cyclopropyl, cyclobutyl, cyclopentyl, cyclopentenyl, cyclohexyl, cyclohexenyl, cycloheptyl, and cyclooctyl.

The term “cycloalkylene” as used herein, means, unless otherwise stated, a divalent radical derived from cycloalkyl, as defined herein.

The term “heterocycloalkyl” as used herein, means a saturated or partially saturated monocyclic, bicyclic or polycyclic ring containing carbon and at least one heteroatom selected from oxygen, nitrogen, and sulfur (O, N, and S) and wherein there are no delocalized pi electrons (aromaticity) shared among the ring carbons or heteroatoms, and wherein the nitrogen and sulfur atoms may optionally be oxidized, and the nitrogen heteroatom may optionally be quaternized. The term “2 to 20 membered heterocycloalkyl” is to be construed accordingly. The heterocyclyl ring structure may be substituted by one or more substituents. The substituents can themselves be optionally substituted. The heterocycloalkyl may be bonded via a carbon atom or heteroatom. The term polycyclic encompasses bridged, fused and spirocyclic heterocycloalkyl.

Examples of heterocycloalkyl rings include, without limitations, oxetanyl, azetidinyl, tetrahydrofuranyl, tetrahydropyranyl, pyrrolidinyl, oxazolinyl, isoxazolinyl, oxazolidinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, 1,4-dioxanyl, dihydrofuranyl, 1,3-dioxolanyl, imidazolidinyl, dihydroisoxazolinyl, pyrrolinyl, pyrazolinyl, oxazepinyl, dithiolanyl, homotropanyl, dihydropyranyl, e.g., 3,6-dihydro-2H-pyranyl, 2-oxaspiro[3.3]heptanyl, e.g., 2-oxaspiro[3.3]heptan-6-yl, 7-oxaspiro[3.5]nonanyl, e.g., 7-oxaspiro[3.5]nonan-2-yl, 1-oxa-7-azaspiro[3.5]nonanyl, 2-azaspiro[3.3]heptanyl, 2,7-diazaspiro[3.5]nonanyl, 7-azaspiro[3.5]nonanyl, and the like.

Examples of 2 to 20 membered heterocycloalkyl include, without limitations, azetidinyl, pyrrolidinyl, tetrahydrofuranyl, tetrahydrothienyl, piperidinyl, piperazinyl, dihydroisoxazolinyl, tetrahydropyranyl, morpholinyl, dihydropyranyl, e.g., 3,6-dihydro-2H-pyranyl, 2-oxaspiro[3.3]heptanyl, e.g., 2-oxaspiro[3.3]heptan-6-yl, 7-oxaspiro[3.5]nonanyl, e.g., 7-oxaspiro[3.5]nonan-2-yl, 1-oxa-7-azaspiro[3.5]nonanyl, 2-azaspiro[3.3]heptanyl, e.g., 2-azaspiro[3.3]heptan-6-yl, 2,7-diazaspiro[3.5]nonanyl, and 7-azaspiro[3.5]nonanyl.

The term “heterocycloalkylene” as used herein, means, unless otherwise stated, a divalent radical derived from heterocycloalkyl, as defined herein.

The term “heteroalkyl”, refers to a straight or branched chain alkyl group, as defined herein, in which one or more of the carbon atoms has been replaced by a heteroatom selected from oxygen, nitrogen, sulfur and phosphorus (O, N, S, and P), and wherein the nitrogen, sulfur and phosphorus heteroatoms may optionally be oxidized, and the nitrogen heteroatom may optionally be quaternized. The term “2 to 20 membered heteroalkyl” is to be construed accordingly. Exemplary heteroalkyls include alkyl ethers, secondary and tertiary alkyl amines, amides, alkyl sulfides, and the like. The group may be a terminal group or a bridging group. The heteroatom(s), e.g., O, N, S, P, may be placed at any interior position of the heteroalkyl group or at the position at which the alkyl group is attached to the remainder of the molecule. Heteroalkyl is an uncyclized chain. The heteroalkyl chain may be substituted by one or more substituents. The substituents can themselves be optionally substituted. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —O—CH₃, —O—CH₂—CH₃, and —CN. Up to two or three heteroatoms may be consecutive, such as, for example, —CH₂—NH—OCH₃. A heteroalkyl moiety may include one heteroatom (e.g., O, N, S, or P). A heteroalkyl moiety may include two optionally different heteroatoms (e.g., O, N, S, or P). A heteroalkyl moiety may include three optionally different heteroatoms (e.g., O, N, S, or P). A heteroalkyl moiety may include four optionally different heteroatoms (e.g., O, N, S, or P). A heteroalkyl moiety may include five optionally different heteroatoms (e.g., O, N, S, or P).

The term “heteroalkylene” as used herein, means, unless otherwise stated, a divalent radical derived from heteroalkyl, as defined herein. Heteroalkylene may be exemplified, but not limited by, —CH₂—CH₂—S—CH₂—CH₂—, —CH₂—S—CH₂—CH₂—NH—CH₂—, —O—CH₂—CH₂—CH₂—CH₂—, —CH₂—CH₂—CH₂—CH₂—CH₂—NH(C═O)—, —O—CH₂—CH₂—CH₂—CH₂—CH₂—CH₂—NH—, and —P(═O)(OH)—O—CH₂—CH₂—CH₂—CH₂—CH₂—CH₂—NH(C═O)—.

The term “heteroalkenyl”, refers to a straight or branched chain alkenyl group, as defined herein, in which one or more of the carbon atoms has been replaced by a heteroatom selected from oxygen, nitrogen, sulfur and phosphorus (O, N, S, and P), and wherein the nitrogen, sulfur and phosphorus heteroatoms may optionally be oxidized, and the nitrogen heteroatom may optionally be quaternized. The term “2 to 20 membered heteroalkenyl” is to be construed accordingly. Exemplary heteroalkenyls include alkenyl ethers, secondary and tertiary alkenyl amines, amides, alkenyl sulfides, and the like. The group may be a terminal group or a bridging group. The heteroatom(s), e.g., O, N, S, P, may be placed at any interior position of the heteroalkenyl group or at the position at which the alkenyl group is attached to the remainder of the molecule. Heteroalkenyl is an uncyclized chain. The heteroalkenyl chain may be substituted by one or more substituents. The substituents can themselves be optionally substituted. Examples include, but are not limited to 3-hydroxy-1-propenyl, 3-aminoprop-1-enyl, 2-aminosulfonylethenyl, 2-methylsulfonylethenyl, and the like. A heteroalkenyl moiety may include one heteroatom, e.g., O, N, S, or P. A heteroalkenyl moiety may include two optionally different heteroatoms (e.g., O, N, S, or P). A heteroalkenyl moiety may include three optionally different heteroatoms (e.g., O, N, S, or P). A heteroalkenyl moiety may include four optionally different heteroatoms (e.g., O, N, S, or P). A heteroalkenyl moiety may include five optionally different heteroatoms (e.g., O, N, S, or P).

The term “heteroalkenylene” as used herein, means, unless otherwise stated, a divalent radical derived from heteroalkenyl, as defined herein.

The terms “halo” or “halogen,” by themselves or as part of another substituent, mean, unless otherwise stated, a fluorine, chlorine, bromine, or iodine atom.

As used herein, the term “aryl” as used herein means monocyclic, bicyclic or polycyclic carbocyclic aromatic rings. Examples of aryl include, but are not limited to, phenyl, naphthyl (e.g., naphth-1-yl, naphth-2-yl), anthryl (e.g., anthr-1-yl, anthr-9-yl), phenanthryl (e.g., phenanthr-1-yl, phenanthr-9-yl), and the like. Aryl is also intended to include monocyclic, bicyclic or polycyclic carbocyclic aromatic rings substituted with carbocyclic aromatic rings. Examples of aryl include, without limitations, biphenyl (e.g., biphenyl-2-yl, biphenyl-3-yl, biphenyl-4-yl), phenylnaphthyl (e.g., 1-phenylnaphth-2-yl, 2-phenylnaphth-1-yl), and the like. Aryl is also intended to include partially saturated bicyclic or polycyclic carbocyclic rings with at least one unsaturated moiety (e.g., a benzo moiety). Examples include, without limitations, indanyl (e.g., indan-1-yl, indan-S-yl), indenyl (e.g., inden-1-yl, inden-S-yl), 1,2,3,4-tetrahydronaphthyl (e.g., 1,2,3,4-tetrahydronaphth-1-yl, 1,2,3,4-tetrahydronaphth-2-yl, 1,2,3,4-tetrahydronaphth-6-yl), 1,2-dihydronaphthyl (e.g., 1,2-dihydronaphth-1-yl, 1,2-dihydronaphth-4-yl, 1,2-dihydronaphth-6-yl), fluorenyl (e.g., fluoren-1-yl, fluoren-4-yl, fluoren-9-yl), and the like. Aryl is also intended to include partially saturated bicyclic or polycyclic carbocyclic aromatic rings containing one or two bridges. Examples include, without limitations, benzonorbornyl (e.g., benzonorborn-3-yl, benzonorborn-6-yl), 1,4-ethano-1,2,3,4-tetrahydronapthyl (e.g., 1,4-ethano-1,2,3,4-tetrahydronapth-2-yl, 1,4-ethano-1,2,3,4-tetrahydronapth-10-yl), and the like. The term “C₆-C₁₂aryl” is to be construed accordingly.

Examples of C₆-C₁₂aryl include, without limitations, phenyl and naphthyl. In an embodiment, C₆-C₁₂aryl is phenyl.

The term “arylene” as used herein, means, unless otherwise stated, a divalent radical derived from aryl, as defined herein.

As used herein, the term “heteroaryl” as used herein is intended to include monocyclic heterocyclic aromatic rings containing one or more heteroatoms selected from oxygen, nitrogen, and sulfur (0, N, and S). Examples include, without limitations, pyrrolyl, furanyl, thienyl, oxazolyl, thiazolyl, imidazolyl, pyrazolyl, isothiazolyl, isooxazolyl, triazolyl, (e.g., 1,2,4-triazolyl), oxadiazolyl, (e.g., 1,2,3-oxadiazolyl, 1,2,4-oxadiazolyl, 1,2,5-oxadiazolyl, 1,3,4-oxadiazolyl), thiadiazolyl (e.g., 1,2,3-thiadiazolyl, 1,2,4-thiadiazolyl, 1,2,5-thiadiazolyl, 1,3,4-thiadiazolyl), tetrazolyl, pyranyl, pyridinyl, pyridazinyl, pyrimidinyl, pyrazinyl, 1,2,3-triazinyl, 1,2,4-triazinyl, 1,3,5-triazinyl, thiadiazinyl, azepinyl, azecinyl, and the like.

Heteroaryl is also intended to include bicyclic heterocyclic aromatic rings containing one or more heteroatoms selected from oxygen, nitrogen, and sulfur (0, N, and S). Examples include, without limitations, indolyl, isoindolyl, benzofuranyl, benzothiophenyl, indazolyl, benzopyranyl, benzimidazolyl, benzothiazolyl, benzisothiazolyl, benzoxazolyl, benzisoxazolyl, benzoxazinyl, benzotriazolyl, naphthyridinyl, phthalazinyl, pteridinyl, purinyl, quinazolinyl, cinnolinyl, quinolinyl, isoquinolinyl, quinoxalinyl, oxazolopyridinyl, isooxazolopyridinyl, pyrrolopyridinyl, furopyridinyl, thienopyridinyl, imidazopyridinyl, imidazopyrimidinyl, pyrazolopyridinyl, pyrazolopyrimidinyl, pyrazolotriazinyl, thiazolopyridinyl, thiazolopyrimidinyl, imdazothiazolyl, triazolopyridinyl, triazolopyrimidinyl, and the like.

Heteroaryl is also intended to include polycyclic heterocyclic aromatic rings containing one or more heteroatoms selected from oxygen, nitrogen, and sulfur (O, N, and S). Examples include, without limitations, carbazolyl, phenoxazinyl, phenazinyl, acridinyl, phenothiazinyl, carbolinyl, phenanthrolinyl, and the like.

Heteroaryl is also intended to include partially saturated monocyclic, bicyclic or polycyclic heterocyclyls containing one or more heteroatoms selected oxygen, nitrogen, and sulfur (O, N, and S). Examples include, without limitations, imidazolinyl, indolinyl, dihydrobenzofuranyl, dihydrobenzothienyl, dihydrobenzopyranyl, dihydropyridooxazinyl, dihydrobenzodioxinyl (e.g., 2,3-dihydrobenzo[b][1,4]dioxinyl), benzodioxolyl (e.g., benzo[d][1,3]dioxole), dihydrobenzooxazinyl (e.g., 3,4-dihydro-2H-benzo[b][1,4]oxazine), tetrahydroindazolyl, tetrahydrobenzimidazolyl, tetrahydroimidazo[4,5-c]pyridyl, tetrahydroquinolinyl, tetrahydroisoquinolinyl, tetrahydroquinoxalinyl, and the like.

The heteroaryl ring structure may be substituted by one or more substituents. The substituents can themselves be optionally substituted. The heteroaryl ring may be bonded via a carbon atom or heteroatom.

The term “5-12 membered heteroaryl” is to be construed accordingly.

Examples of 5-12 membered heteroaryl include, without limitations, indolyl, imidazopyridyl, isoquinolinyl, benzooxazolonyl, pyridinyl, pyrimidinyl, pyridinonyl, benzotriazolyl, pyridazinyl, pyrazolotriazinyl, indazolyl, benzimidazolyl, quinolinyl, triazolyl, (e.g., 1,2,4-triazolyl), pyrazolyl, thiazolyl, oxazolyl, isooxazolyl, pyrrolyl, oxadiazolyl, (e.g., 1,2,3-oxadiazolyl, 1,2,4-oxadiazolyl, 1,2,5-oxadiazolyl, 1,3,4-oxadiazolyl), imidazolyl, pyrrolopyridinyl, tetrahydroindazolyl, quinoxalinyl, thiadiazolyl (e.g., 1,2,3-thiadiazolyl, 1,2,4-thiadiazolyl, 1,2,5-thiadiazolyl, 1,3,4-thiadiazolyl), pyrazinyl, oxazolopyridinyl, pyrazolopyrimidinyl, benzoxazolyl, indolinyl, isooxazolopyridinyl, dihydropyridooxazinyl, tetrazolyl, dihydrobenzodioxinyl (e.g., 2,3-dihydrobenzo[b][1,4]dioxinyl), benzodioxolyl (e.g., benzo[d][1,3]dioxole) and dihydrobenzooxazinyl (e.g., 3,4-dihydro-2H-benzo[b][1,4]oxazine).

The term “heteroarylene” as used herein, means, unless otherwise stated, a divalent radical derived from heteroaryl, as defined herein.

The symbol “ [symbol] ” denotes the point of attachment of a chemical moiety to the remainder of a molecule or chemical formula.

“Amino” means a substituent containing at least one nitrogen atom, e.g., NH₂.

“Alkylamino” means an amino or NH₂ group where one of the hydrogens is replaced with an alkyl group, e.g., —NH(alkyl). Examples of alkylamino groups include, but are not limited to, methylamino, e.g., —NH(CH₃), ethylamino, propylamino, iso-propylamino, n-butylamino, sec-butylamino, tert-butylamino, etc.

The term “oxo,” as used herein, means an oxygen that is double bonded to a carbon atom.

The term “mercapto” as used herein, means —SH.

“Hydroxymethyl” means a —CH₂OH group.

Each of the above terms, e.g., “alkyl,” “heteroalkyl,” “cycloalkyl,” “heterocycloalkyl,” “aryl,” and “heteroaryl”, include both substituted and unsubstituted forms of the indicated radical. Preferred substituents for each type of radical are provided below.

Substituents for the alkyl, heteroalkyl, cycloalkyl, heterocycloalkyl, aryl, heteroaryl radicals (including those groups referred to as alkylene, heteroalkylene, cycloalkylene, heterocycloalkylene, arylene, and heteroarylene) can be one or more of a variety of groups selected from the following moieties:
(A) oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl, heteroalkyl, cycloalkyl, heterocycloalkyl, aryl, heteroaryl, substituted with at least one substituent selected from:
(i) oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(ii) alkyl, heteroalkyl, cycloalkyl, heterocycloalkyl, aryl, heteroaryl, substituted with at least one substituent selected from:
(a) oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(b) alkyl, heteroalkyl, cycloalkyl, heterocycloalkyl, aryl, heteroaryl, substituted with at least one substituent selected from: oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, substituents for the alkyl, heteroalkyl, cycloalkyl, heterocycloalkyl, aryl, heteroaryl radicals (including those groups referred to as alkylene, heteroalkylene, cycloalkylene, heterocycloalkylene, arylene, and heteroarylene) can be one or more of a variety of groups selected from the following moieties:
oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, e.g., C₁-C₈ alkyl, C₁-C₆ alkyl, or C₁-C₄ alkyl, unsubstituted heteroalkyl, e.g., 2 to 8 membered heteroalkyl, 2 to 6 membered heteroalkyl, or 2 to 4 membered heteroalkyl, unsubstituted cycloalkyl, e.g., C₃-C₈ cycloalkyl, C₃-C₆ cycloalkyl, or C₅-C₆ cycloalkyl, unsubstituted heterocycloalkyl, e.g., 3 to 8 membered heterocycloalkyl, 3 to 6 membered heterocycloalkyl, or 5 to 6 membered heterocycloalkyl, unsubstituted aryl, e.g., C₆-C₁₀ aryl, C₁₀ aryl, or phenyl, unsubstituted heteroaryl, e.g., 5 to 10 membered heteroaryl, 5 to 9 membered heteroaryl, or 5 to 6 membered heteroaryl, unsubstituted alkoxy, e.g., C₁-C₈ alkoxy, C₁-C₆ alkoxy, or C₁-C₄ alkoxy, unsubstituted alkylthio, e.g., C₁-C₈ alkylthio, C₁-C₆ alkylthio, or C₁-C₄ alkylthio, and unsubstituted alkylamino, e.g., C₁-C₈ alkylamino, C₁-C₆ alkylamino, or C₁-C₄ alkylamino.

In embodiments, substituents for the alkyl, heteroalkyl, cycloalkyl, heterocycloalkyl, aryl, heteroaryl radicals (including those groups referred to as alkylene, heteroalkylene, cycloalkylene, heterocycloalkylene, arylene, and heteroarylene) can be one or more of a variety of groups selected from the following moieties:
hydroxyl, carboxyl, oxo, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, substituents for the alkyl, heteroalkyl, cycloalkyl, heterocycloalkyl, aryl, heteroaryl radicals (including those groups referred to as alkylene, heteroalkylene, cycloalkylene, heterocycloalkylene, arylene, and heteroarylene) can be one or more of a variety of groups selected from the following moieties: halogen, hydroxyl, carboxyl, oxo, and amino.

“Analog” or “analogue” is used in accordance with its plain ordinary meaning within Chemistry and Biology and refers to a chemical compound that is structurally similar to another compound, i.e., a so-called “reference” compound, but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

As used herein, the term “isomers” refers to compounds having the same number and kind of atoms, and hence the same molecular weight, but differing in respect to the structural arrangement or configuration of the atoms.

The term “tautomer”, as used herein, refers to one of two or more structural isomers which exist in equilibrium and which are readily converted from one isomeric form to another.

Certain compounds provided herein possess asymmetric carbon atoms (optical or chiral centers) or double bonds; the enantiomers, racemates, diastereomers, tautomers, geometric isomers, stereoisomers that may be defined, in terms of absolute stereochemistry, as (R)- or (S)- or, as (D)- or (L)- for amino acids, and individual isomers are encompassed within the scope of the present disclosure. Compounds provided herein include those in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

It will be apparent to one skilled in the art that certain compounds provided herein may exist in tautomeric forms, all such tautomeric forms of the compounds being within the scope of the present disclosure.

Unless otherwise stated, structures depicted herein are also meant to include compounds which differ only in the presence of one or more isotopically enriched atoms. Thus, the compounds provided herein may also contain unnatural proportions of atomic isotopes at one or more of the atoms that constitute such compounds. Isotopic substitutions encompassed by the compounds disclosed herein include, but are not limited to, ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging. All isotopic variations of the compounds provided herein, whether radioactive or not, are included within the present disclosure.

The description of compounds provided herein is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

### ENUMERATED EMBODIMENTS

Embodiment 1. A double stranded RNAi (dsRNAi) agent, or a pharmaceutically acceptable salt thereof, of formula (I)

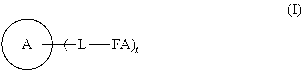

wherein:
A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
L is a linker;
FA is a fatty acid moiety capable of facilitating delivery of the double stranded nucleic acid into a cell; and
t is an integer from 1 to 5.

Embodiment 2. The dsRNAi agent of Embodiment 1, or a pharmaceutically acceptable salt thereof, wherein the sense strand comprises a nucleic acid sequence that is at least about 80% identical to SEQ ID NO: 1, as measured over the entirety of the sense strand.

Embodiment 3. The dsRNAi agent of Embodiment 1 or 2, or a pharmaceutically acceptable salt thereof, wherein the sense strand has a nucleic acid sequence that is at least about 85%, at least about 90%, at least about 95% or is 100% identical to SEQ ID NO: 1, as measured over the entirety of the sense strand.

Embodiment 4. The dsRNAi agent of any one of the preceding Embodiments, or a pharmaceutically acceptable salt thereof, wherein the sense strand comprises a region of sequence identity to an mRNA encoding DMPK, and wherein the region of sequence identity comprises at least 8 contiguous nucleotides of SEQ ID NO: 1.

Embodiment 5. The dsRNAi agent of Embodiment 4, or a pharmaceutically acceptable salt thereof, wherein the region of sequence identity comprises at least 9, at least 10, at least 11, at least 12, at least 13, at least 14, at least 15, at least 16, at least 17, at least 18, at least 19, or at least 20 contiguous nucleotides of SEQ ID NO: 1.

Embodiment 6. The dsRNAi agent of any one of the preceding Embodiments, or a pharmaceutically acceptable salt thereof, wherein each of the antisense strand and the sense strand is independently 17 to 25 nucleotides in length.

Embodiment 7. The dsRNAi agent of any one of the preceding Embodiments, or a pharmaceutically acceptable salt thereof, wherein the antisense strand comprises a nucleic acid sequence that is at least about 80%, at least about 85%, at least about 90%, at least about 95%, at least about 96%, at least about 97%, at least about 98%, at least about 99%, or is 100% complementary to SEQ ID NO: 1, as measured over the entirety of the antisense strand.

Embodiment 8. The dsRNAi agent of any one of the preceding Embodiments, or a pharmaceutically acceptable salt thereof, wherein the nucleic acid sequence of the sense strand has no more than two mismatches to the nucleic acid sequence of the antisense strand.

Embodiment 9. The dsRNAi agent of any one of the preceding Embodiments, or a pharmaceutically acceptable salt thereof, wherein the nucleic acid sequence of the sense strand has no more than one mismatch to the nucleic acid sequence of the antisense strand.

Embodiment 10. The dsRNAi agent of any one of the preceding Embodiments, or a pharmaceutically acceptable salt thereof, wherein the nucleic acid sequence of the sense strand has no mismatches to the nucleic acid sequence of the antisense strand.

Embodiment 11. The dsRNAi agent of any one of the preceding Embodiments, wherein the antisense strand comprises at least 15 contiguous nucleotides differing by no more than 3 nucleotides from any one of the antisense sequences listed in SEQ ID NO 294-584.

Embodiment 12. The dsRNAi agent of any one of the preceding Embodiments, wherein the sense strand comprises at least 15 contiguous nucleotides differing by no more than 3 nucleotides from any one of the sense sequences listed in SEQ ID NO 3-293.

Embodiment 13. The dsRNAi agent of any one of the preceding Embodiments, wherein the antisense strand comprises at least 17, at least 18, or at least 19 contiguous nucleotides differing by no more than 1 nucleotide from any one of the antisense sequences listed in SEQ ID NO 294-584.

Embodiment 14. The dsRNAi agent of any one of the preceding Embodiments, wherein the sense strand comprises at least 17, at least 18, or at least 19 contiguous nucleotides differing by no more than 1 nucleotide from any one of the sense sequences listed in SEQ ID NO 3-293.

Embodiment 15. The dsRNAi agent of any one of the preceding Embodiments, wherein the antisense strand comprises any one of the antisense sequences listed in SEQ ID NO 294-584.

Embodiment 16. The dsRNAi agent of any one of the preceding Embodiments, wherein the sense strand comprises any one of the sense sequences listed in SEQ ID NO 3-293.

Embodiment 17. The dsRNAi agent of any one of the preceding Embodiments, wherein the nucleic acid sequence of the antisense strand consists of any one of the antisense sequences listed in SEQ ID NO 294-584.

Embodiment 18. The dsRNAi agent of any one of the preceding Embodiments, wherein the nucleic acid sequence of the sense strand consists of any one of the sense sequences listed in SEQ ID NO 3-293.

Embodiment 19. The dsRNAi agent of any one of the preceding Embodiments, wherein at least one of the nucleotides of the sense strand and the antisense strand is a modified nucleotide.

Embodiment 20. The dsRNAi agent of any one of the preceding Embodiments, wherein all the nucleotides of the sense strand and the antisense strand are modified nucleotides.

Embodiment 21. The dsRNAi agent of any one of the preceding Embodiments, wherein all the nucleotides of the sense strand and the antisense strand comprise a 2′F or 2′-OMe modification.

Embodiment 22. The dsRNAi agent of any one of the preceding Embodiments, or a pharmaceutically acceptable salt thereof, wherein the linker is attached to the sense strand.

Embodiment 23. The dsRNAi agent of any one of the preceding Embodiments, or a pharmaceutically acceptable salt thereof, wherein the linker is attached to the antisense strand.

Embodiment 24. The dsRNAi agent of any one of the preceding Embodiments, or a pharmaceutically acceptable salt thereof, wherein the linker is attached to the 3′ terminus of the sense strand or antisense strand

Embodiment 25. The dsRNAi agent of any one of the preceding Embodiments, or a pharmaceutically acceptable salt thereof, wherein the linker is attached to the 5′ terminus of the sense strand or antisense strand.

Embodiment 26. The dsRNAi agent of any one of the preceding Embodiments, wherein the fatty acid moiety has binding activity in a cell, e.g., in a muscle cell.

Embodiment 27. The dsRNAi agent of any one of the preceding Embodiments, wherein the fatty acid moiety has a lipoprotein binding activity in a cell, e.g., in a muscle cell, e.g., in a skeletal muscle cell.

Embodiment 28. The dsRNAi agent of any one of the preceding Embodiments, wherein the fatty acid moiety has an albumin binding activity in a cell, e.g., in a muscle cell, e.g., in a skeletal muscle cell.

Embodiment 29. The dsRNAi agent of any one of the preceding Embodiments, wherein the fatty acid moiety is capable of facilitating systemic delivery into one or more cells, e.g., one or more muscle cells.

Embodiment 30. The dsRNAi agent of any one of the preceding Embodiments, wherein the fatty acid moiety is capable of increasing the in-vivo half-life and/or is capable of changing the biodistribution properties of the double stranded nucleic acid within one or more target tissues, e.g., muscle and/or nerve.

Embodiment 31. The dsRNAi agent of any one of the preceding Embodiments, or a pharmaceutically acceptable salt thereof, wherein the fatty acid moiety provides increased biodistribution within one or more target tissues, e.g., muscle and/or nerve.

Embodiment 32. The dsRNAi agent of any one of the preceding Embodiments, wherein the one or more target tissues are disease-relevant tissues.

Embodiment 33. The dsRNAi agent of any one of the preceding Embodiments, or a pharmaceutically acceptable salt thereof, wherein the one or more target tissues is selected from tissues of brain, spinal cord, muscle, spleen, lung, heart, liver, bladder, and kidney.

Embodiment 34. The dsRNAi agent of any one of the preceding Embodiments, or a pharmaceutically acceptable salt thereof, wherein the one or more target tissues is selected from the group consisting of prefrontal cortex, cerebellum, and cerebrum; cervical, thoracic and lumbar in spinal cord; heart, bicep, tricep, semitendinosus, platysma, gluteus, tibialis anterior, quadriceps, and/or gastrocnemius.

Embodiment 35. The dsRNAi agent of any one of the preceding Embodiments, wherein the fatty acid moiety improves the stability, bioavailability, biodistribution, and/or cellular uptake of the double stranded nucleic acid as compared to a control, e.g., the double stranded nucleic acid without the fatty acid moiety.

Embodiment 36. The dsRNAi agent of any one of the preceding Embodiments, or a pharmaceutically acceptable salt thereof, wherein the fatty acid moiety comprises a multibranched structure.

Embodiment 37. The dsRNAi agent of any one of the preceding Embodiments, or a pharmaceutically acceptable salt thereof, wherein the fatty acid moiety comprises 1 to 3 fatty acid chains.

Embodiment 38. The dsRNAi agent of any one of the preceding Embodiments, or a pharmaceutically acceptable salt thereof, wherein the fatty acid moiety comprises 2 fatty acid chains.

Embodiment 39. The dsRNAi agent of any one of Embodiments 37 and 38, or a pharmaceutically acceptable salt thereof, wherein the fatty acid chains each comprise an amide-containing alkyl group.

Embodiment 40. The dsRNAi agent of any one of the preceding Embodiments, or a pharmaceutically acceptable salt thereof, wherein the fatty acid moiety comprises an alpha amino acid scaffold or a part thereof.

Embodiment 41. The dsRNAi agent of any one of the preceding Embodiments, or a pharmaceutically acceptable salt thereof, wherein the linker is directly attached to the double stranded nucleic acid or indirectly through one or more groups selected from —O—, —NH—, —N(C₁-C₁₀ alkyl)-, —S—, —C(O)—, —C(O)O—, —OC(O)—, —C(O)NH—, —NHC(O)—, —OPO₂O—, and —OP(S)(O)O—.

Embodiment 42. The dsRNAi agent of any one of the preceding Embodiments, or a pharmaceutically acceptable salt thereof, wherein the linker comprises a functional group selected from the group consisting of —NH—, —N(C₁-C₁₀ alkyl)-, —O—, —S—, —C(O)—, —NHC(O)—, —NHC(O)NH—, —C(O)O—, —OC(O)—, —C(O)NH—, —OPO₂O—, —OP(S)(O)O—, substituted or unsubstituted C₁-C₂₀ alkylene, substituted or unsubstituted C₂-C₂₀ alkenylene, substituted or unsubstituted 2 to 20 membered heteroalkylene, substituted or unsubstituted C₃-C₁₀ cycloalkylene, substituted or unsubstituted 2 to 20 membered heterocycloalkylene, substituted or unsubstituted C₆-C₁₂ arylene, and substituted or unsubstituted 5 to 12 membered heteroarylene.

Embodiment 43. The dsRNAi agent of any one of the preceding Embodiments, or a pharmaceutically acceptable salt thereof, wherein the linker comprises a substituted or unsubstituted C₁-C₂₀ alkylene or substituted or unsubstituted 2 to 20 membered heteroalkylene

Embodiment 44. The dsRNAi agent of Embodiment 42 or 43, or a pharmaceutically acceptable salt thereof, wherein the alkylene, alkenylene, heteroalkylene, cycloalkylene, heterocycloalkylene, arylene, and heteroarylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

Embodiment 45. The dsRNAi agent of any one of the preceding Embodiments, or a pharmaceutically acceptable salt thereof, wherein the linker comprises a group selected from the group consisting

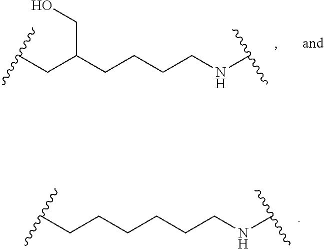 [SMILES: *CC(CO)CCCCN* ; *CCCCCCN*]

Embodiment 46. The dsRNAi agent of any one of the preceding Embodiments, or a pharmaceutically acceptable salt thereof, wherein the linker is selected from the group consisting of

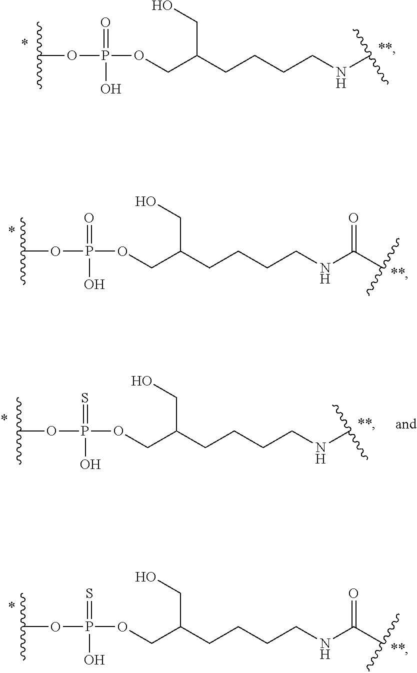 [SMILES: *NCCCCC(CO)COP(=O)(O)O* ; *OP(=O)(O)OCC(CO)CCCCNC(*)=O ; *NCCCCC(CO)COP(O)(=S)O* ; *OP(O)(=S)OCC(CO)CCCCNC(*)=O]

wherein * indicates the point of attachment to the double stranded nucleic acid and ** indicates the point of attachment to the fatty acid moiety.

Embodiment 47. The dsRNAi agent of Embodiment 46, or a pharmaceutically acceptable salt thereof, wherein the * indicates the point of attachment to a 3′ carbon of a terminal 3′ nucleotide or a 5′ carbon of a terminal 5′ nucleotide of the sense strand of the double stranded nucleic acid.

Embodiment 48. The dsRNAi agent of any one of the preceding Embodiments, or a pharmaceutically acceptable salt thereof, wherein the linker is a group of formula

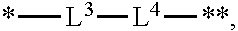

wherein
L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)O—, substituted or unsubstituted C₁-C₂₀ alkylene, substituted or unsubstituted C₂-C₂₀ alkenylene, substituted or unsubstituted 2 to 20 membered heteroalkylene, substituted or unsubstituted C₃-C₁₀ cycloalkylene, substituted or unsubstituted 2 to 20 membered heterocycloalkylene, substituted or unsubstituted C₆-C₁₂ arylene or substituted or unsubstituted 5 to 12 membered heteroarylene, and
wherein * indicates the point of attachment to the double stranded nucleic acid and ** indicates the point of attachment to the fatty acid moiety.

Embodiment 49. The dsRNAi agent of Embodiment 48, or a pharmaceutically acceptable salt thereof, wherein L³ and L⁴ are each independently selected from a bond, —NH—, —O—, —S—, —C(O)—, —NHC(O)—, —NHC(O)NH—, —C(O)O—, —OC(O)—, —C(O)NH—, —OPO₂—O—, —OP(S)(O)O—, substituted or unsubstituted C₁-C₁₂ alkylene, substituted or unsubstituted C₂-C₁₂ alkenylene, and substituted or unsubstituted 2 to 20 membered heteroalkylene.

Embodiment 50. The dsRNAi agent of Embodiment 48 or 49, or a pharmaceutically acceptable salt thereof, wherein the alkylene, alkenylene, heteroalkylene, cycloalkylene, heterocycloalkylene, arylene and heteroarylene are each independently unsubstituted or substituted with 1, 2, 3, 4 or 5 substituents independently selected from the group consisting of hydroxyl, carboxyl, oxo, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

Embodiment 51. The dsRNAi agent of any one of Embodiments 48 to 50, or a pharmaceutically acceptable salt thereof, wherein the sense strand is covalently attached to L³.

Embodiment 52. The dsRNAi agent of any one of Embodiments 48 to 51, or a pharmaceutically acceptable salt thereof, wherein L³ is attached to a 3′ carbon of a 3′ terminal nucleotide of the sense strand of the double stranded nucleic acid.

Embodiment 53. The dsRNAi agent of any one of Embodiments 48 to 51, or a pharmaceutically acceptable salt thereof, wherein L³ is attached to a 5′ carbon of a 5′ terminal nucleotide of the sense strand of the double stranded nucleic acid.

Embodiment 54. The dsRNAi agent of any one of Embodiments 48 to 51, or a pharmaceutically acceptable salt thereof, wherein L³ is attached to a 5′ carbon of a 5′ terminal nucleotide and a 3′ carbon of a 3′ terminal nucleotide of the sense strand of the double stranded nucleic acid.

Embodiment 55. The dsRNAi agent of any one of Embodiments 48 to 52, or a pharmaceutically acceptable salt thereof, wherein t is 1 and L³ is attached to a 3′ carbon of a 3′ terminal nucleotide of the sense strand of the double stranded nucleic acid.

Embodiment 56. The dsRNAi agent of any one of Embodiments 48 to 55, or a pharmaceutically acceptable salt thereof, wherein L³ is independently a bond.

Embodiment 57. The dsRNAi agent of any one of Embodiments 48 to 55, or a pharmaceutically acceptable salt thereof, wherein L³ is independently —OPO₂—O—.

Embodiment 58. The dsRNAi agent of any one of Embodiments 48 to 55, or a pharmaceutically acceptable salt thereof, wherein L³ is independently —O—.

Embodiment 59. The dsRNAi agent of any one of Embodiments 48 to 58, or a pharmaceutically acceptable salt thereof, wherein L⁴ is independently substituted or unsubstituted C₁-C₂₀ alkylene or substituted or unsubstituted 2 to 20 membered heteroalkylene.

Embodiment 60. The dsRNAi agent of Embodiment 59, or a pharmaceutically acceptable salt thereof, wherein the alkylene and heteroalkylene are each independently unsubstituted or substituted with 1, 2, 3, 4 or 5 substituents independently selected from the group consisting of hydroxyl, carboxyl, oxo, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

Embodiment 61. The dsRNAi agent of any one of Embodiments 48 to 60, or a pharmaceutically acceptable salt thereof, wherein L⁴ is independently -L⁷-NH—C(O)— or -L⁷-C(O)—NH—, wherein L⁷ is substituted or unsubstituted C₁-C₂₀ alkylene.

Embodiment 62. The dsRNAi agent of Embodiment 61, or a pharmaceutically acceptable salt thereof, wherein the alkylene of L⁷ is unsubstituted or substituted with 1, 2 or 3 substituents independently selected from the group consisting of halogen, hydroxyl, carboxyl, oxo, and amino.

Embodiment 63. The dsRNAi agent of any one of Embodiments 48 to 62, or a pharmaceutically acceptable salt thereof, wherein L⁴ is independently

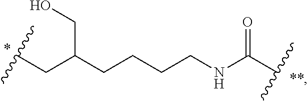 [SMILES: *CC(CO)CCCCNC(*)=O]

wherein * indicates the point of attachment to L³ and ** indicates the point of attachment to the fatty acid moiety.

Embodiment 64. The dsRNAi agent of any one of Embodiments 48 to 62, or a pharmaceutically acceptable salt thereof, wherein L⁴ is independently

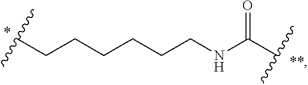 [SMILES: *CCCCCCNC(*)=O]

wherein * indicates the point of attachment to L³ and ** indicates the point of attachment to the fatty acid moiety.

Embodiment 65. The dsRNAi agent of any one of Embodiments 48 to 62, or a pharmaceutically acceptable salt thereof, wherein -L³-L⁴- is independently —O-L⁷-NH—C(O)—, wherein L⁷ is independently substituted or unsubstituted C₅-C₈alkylene Embodiment 66. The dsRNAi agent of Embodiment 65, or a pharmaceutically acceptable salt thereof, wherein the alkylene of L⁷ is unsubstituted or substituted with 1, 2 or 3 substituents independently selected from the group consisting of halogen, hydroxyl, carboxyl, oxo, and amino.

Embodiment 67. The dsRNAi agent of Embodiment 65 or 66, or a pharmaceutically acceptable salt thereof, wherein -L³-L⁴- is independently

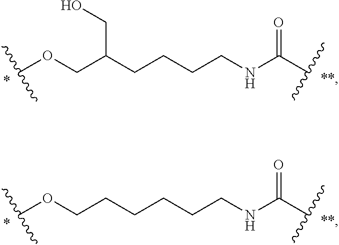 [SMILES: *OCC(CO)CCCCNC(*)=O ; *OCCCCCCNC(*)=O]

wherein * indicates the point of attachment to the double stranded nucleic acid and ** indicates the point of attachment to the fatty acid moiety.

Embodiment 68. The dsRNAi agent of any one of Embodiments 48 to 62, or a pharmaceutically acceptable salt thereof, wherein -L³-L⁴- is independently —OPO₂—O-L⁷-NH—C(O)—, wherein L⁷ is independently substituted or unsubstituted C₅-C₈alkylene.

Embodiment 69. The dsRNAi agent of Embodiment 68, or a pharmaceutically acceptable salt thereof, wherein the alkylene of L⁷ is unsubstituted or substituted with 1, 2 or 3 substituents independently selected from the group consisting of halogen, hydroxyl, carboxyl, oxo, and amino.

Embodiment 70. The dsRNAi agent of any one of Embodiments 68 and 69, or a pharmaceutically acceptable salt thereof, wherein -L³-L⁴- is independently

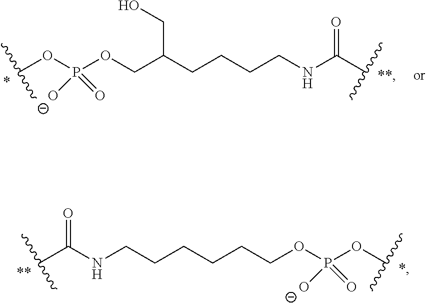 [SMILES: *OP(=O)([O-])OCC(CO)CCCCNC(*)=O ; *OP(=O)([O-])OCCCCCCNC(*)=O]

wherein * indicates the point of attachment to the double stranded nucleic acid and ** indicates the point of attachment to the fatty acid moiety.

Embodiment 71. The dsRNAi agent of Embodiment 70, or a pharmaceutically acceptable salt thereof, wherein -L³-L⁴- is independently

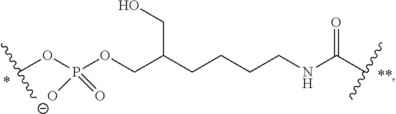 [SMILES: *OP(=O)([O-])OCC(CO)CCCCNC(*)=O]

wherein
* represents attachment to a 3′ carbon of the sense strand

Embodiment 72. The dsRNAi agent of Embodiment 70, or a pharmaceutically acceptable salt thereof, wherein -L³-L⁴- is independently

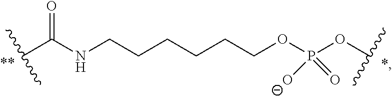 [SMILES: *OP(=O)([O-])OCCCCCCNC(*)=O]

wherein
* represents attachment to a 5′ carbon of the sense strand.

Embodiment 73. The dsRNAi agent of any one of the preceding Embodiments, or a pharmaceutically acceptable salt thereof, wherein the fatty acid moiety comprises a group of formula

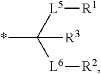

wherein
L⁵ is -L^{5A}-L^{5B}-L^{5C}-L^{5D}-L^{5E}-;
L⁶ is -L^{6A}-L^{6B}-L^{6C}-L^{6D}-L^{6E}-;
L^{5A}, L^{5B} L^{5C}, L^{5D} L^{5E} L^{6A}, L^{6B} L⁶C, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted C₁-C₂₀ alkylene, substituted or unsubstituted 2 to 20 membered heteroalkylene, substituted or unsubstituted C₃-C₁₀ cycloalkylene, substituted or unsubstituted 2 to 20 membered heterocycloalkylene, substituted or unsubstituted C₆-C₁₂ arylene or substituted or unsubstituted 5 to 12 membered heteroarylene;
R¹ and R² are independently unsubstituted or substituted C₁-C₂₅ alkyl or C₂-C₂₅ alkenyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted C₁-C₂₀ alkyl, substituted or unsubstituted 2 to 20 membered heteroalkyl, substituted or unsubstituted C₃-C₁₀ cycloalkyl, substituted or unsubstituted 2 to 20 membered heterocycloalkyl, substituted or unsubstituted C6-C₁₂ aryl, or substituted or unsubstituted 5 to 12 membered heteroaryl; wherein * indicates the point of attachment to the rest of the molecule.

Embodiment 74. The dsRNAi agent of Embodiment 73, or a pharmaceutically acceptable salt thereof, wherein the alkylene, heteroalkylene, cycloalkylene, heterocycloalkylene, arylene, heteroarylene, alkyl, heteroalkyl, cycloalkyl, heterocycloalkyl, aryl, and heteroaryl are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

Embodiment 75. The dsRNAi agent of Embodiment 73 or 74, or a pharmaceutically acceptable salt thereof, wherein R³ is hydrogen.

Embodiment 76. The dsRNAi agent of any one of the preceding Embodiments, or a pharmaceutically acceptable salt thereof, wherein t is 1, 2 or 3, e.g., t is 1.

Embodiment 77. The dsRNAi agent of any one of Embodiments 73 to 76, or a pharmaceutically acceptable salt thereof, wherein
L^{6A} is independently a bond or unsubstituted C₁-C₈ alkylene;
L^{6B} is independently a bond, —NHC(O)—, or unsubstituted phenylene;
L⁶c is independently a bond, unsubstituted C₂-C₈ alkynylene, or unsubstituted phenylene;
L^{6D} is independently a bond or unsubstituted C₁-C₈ alkylene; and
L^{6E} is independently a bond or —NHC(O)—.

Embodiment 78. The dsRNAi agent of any one of Embodiments 73 to 77, or a pharmaceutically acceptable salt thereof, wherein L⁶ is independently —NHC(O)—, —C(O)NH—, substituted or unsubstituted C₁-C₂₀ alkylene, or substituted or unsubstituted 2 to 20 membered heteroalkylene.

Embodiment 79. The dsRNAi agent of Embodiment 78, or a pharmaceutically acceptable salt thereof, wherein the alkylene and heteroalkylene of L⁶ are each independently unsubstituted or substituted with 1, 2 or 3 substituents independently selected from the group consisting of halogen, hydroxyl, carboxyl, oxo, and amino.

Embodiment 80. The dsRNAi agent of any one of Embodiments 73 to 79, or a pharmaceutically acceptable salt thereof, wherein L⁶ is independently a bond, —NIC(O)—,

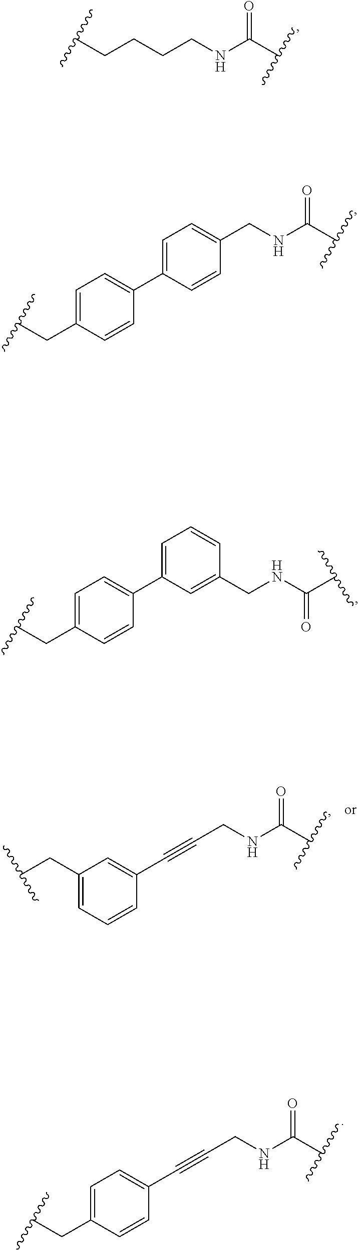 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment 81. The dsRNAi agent of any one of Embodiments 73 to 80, or a pharmaceutically acceptable salt thereof, wherein L⁶ is independently —NHC(O)—.

Embodiment 82. The dsRNAi agent of any one of Embodiments 73 to 81, wherein
L^{5A} is independently a bond or unsubstituted C₁-C₈ alkylene;
L^{5B} is independently a bond, —NHC(O)—, or unsubstituted phenylene;
L^{5C} is independently a bond, unsubstituted C₂-C₅ alkynylene, or unsubstituted phenylene;
L^{5D} is independently a bond or unsubstituted C₁-C₈ alkylene; and
L^{5E} is independently a bond or —NHC(O)—.

Embodiment 83. The dsRNAi agent of any one of Embodiments 73 to 82, or a pharmaceutically acceptable salt thereof, wherein L⁵ is independently —NHC(O)—, —C(O)NH, substituted or unsubstituted C₁-C₂₀ alkylene, or substituted or unsubstituted 2 to 20 membered heteroalkylene.

Embodiment 84. The dsRNAi agent of Embodiment 83, or a pharmaceutically acceptable salt thereof, wherein the alkylene and heteroalkylene are each independently unsubstituted or substituted with 1, 2 or 3 substituents independently selected from the group consisting of halogen, hydroxyl, carboxyl, oxo, and amino.

Embodiment 85. The dsRNAi agent of any one of Embodiments 73 to 84, or a pharmaceutically acceptable salt thereof, wherein L⁵ is independently —NHC(O)—.

Embodiment 86. The dsRNAi agent of any one of Embodiments 73 to 84, or a pharmaceutically acceptable salt thereof, wherein L⁵ is independently a bond,

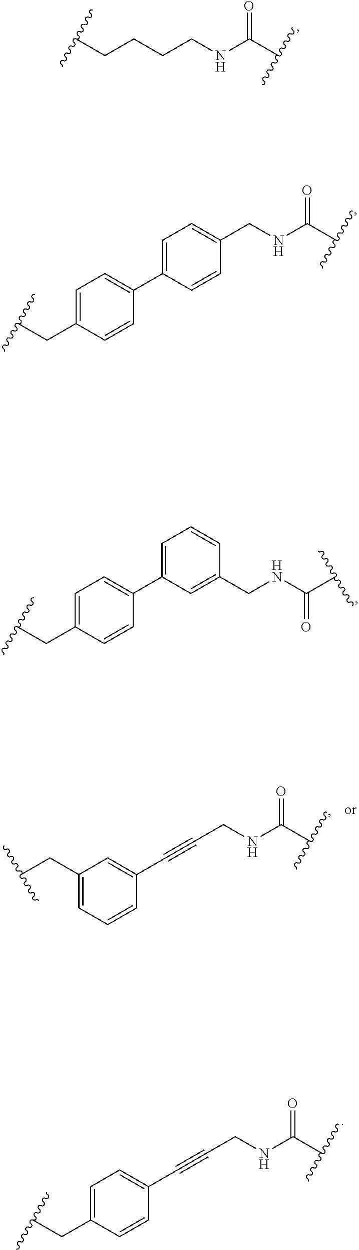 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

Embodiment 87. The dsRNAi agent of any one of Embodiments 73 to 86, or a pharmaceutically acceptable salt thereof, wherein R¹ is unsubstituted C₁-C₁₇ alkyl.

Embodiment 88. The dsRNAi agent of Embodiment 87, or a pharmaceutically acceptable salt thereof, wherein R¹ is unsubstituted C₁-C₁₇ alkyl.

Embodiment 89. The dsRNAi agent of Embodiment 88, or a pharmaceutically acceptable salt thereof, wherein R¹ is unsubstituted C₁₂-C₁₆ alkyl.

Embodiment 90. The dsRNAi agent of Embodiment 89, or a pharmaceutically acceptable salt thereof, wherein R¹ is unsubstituted C13 alkyl.

Embodiment 91. The dsRNAi agent of any one of Embodiments 73 to 87, or a pharmaceutically acceptable salt thereof, wherein R¹ is unsubstituted unbranched C₁-C₁₇ alkyl.

Embodiment 92. The dsRNAi agent of Embodiment 91, or a pharmaceutically acceptable salt thereof, wherein R¹ is unsubstituted unbranched C₁-C₁₇ alkyl Embodiment 93. The dsRNAi agent of Embodiment 92, or a pharmaceutically acceptable salt thereof, wherein R¹ is unsubstituted unbranched C₁₂-C₁₆ alkyl Embodiment 94. The dsRNAi agent of Embodiment 93, or a pharmaceutically acceptable salt thereof, wherein R¹ is unsubstituted unbranched C13 alkyl.

Embodiment 95. The dsRNAi agent of any one of Embodiments 73 to 94, or a pharmaceutically acceptable salt thereof, wherein R² is unsubstituted C₁-C₁₇ alkyl.

Embodiment 96. The dsRNAi agent of Embodiment 95, or a pharmaceutically acceptable salt thereof, wherein R² is unsubstituted C₁-C₁₇ alkyl.

Embodiment 97. The dsRNAi agent of Embodiment 96, or a pharmaceutically acceptable salt thereof, wherein R² is unsubstituted C₁₂-C₁₆ alkyl.

Embodiment 98. The dsRNAi agent of Embodiment 97, or a pharmaceutically acceptable salt thereof, wherein R² is unsubstituted C13 alkyl.

Embodiment 99. The dsRNAi agent of any one of Embodiments 73 to 95, or a pharmaceutically acceptable salt thereof, wherein R² is unsubstituted unbranched C₁-C₁₇ alkyl.

Embodiment 100. The dsRNAi agent of Embodiment 99, or a pharmaceutically acceptable salt thereof, wherein R² is unsubstituted unbranched C₁-C₁₇ alkyl Embodiment 101. The dsRNAi agent of Embodiment 100, or a pharmaceutically acceptable salt thereof, wherein R² is unsubstituted unbranched C₁₂-C₁₆ alkyl.

Embodiment 102. The dsRNAi agent of Embodiment 101, or a pharmaceutically acceptable salt thereof, wherein R² is unsubstituted unbranched C13 alkyl.

Embodiment 103. The dsRNAi agent of any one of the preceding Embodiments, or a pharmaceutically acceptable salt thereof, wherein the fatty acid moiety is a group of formula

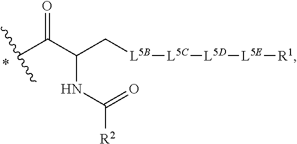

wherein L^{5B} L^{5C}, L^{5D}, L^{5E}, R¹ and R² are defined of any one of Embodiments 73 to 103; and * indicates the point of attachment to the linker.

Embodiment 104. The dsRNAi agent of any one of the preceding Embodiments, or a pharmaceutically acceptable salt thereof, wherein the fatty acid moiety is a group of formula

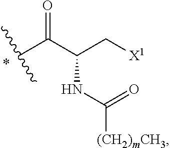

wherein
X¹ is

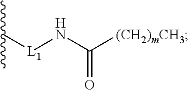

L¹ is —(CH₂)ₙ—, —(CH₂)ₙL²(CH₂)ₙ—, or a bond; L² is —C(═O)NH—, —C(═O)O—, —OC(═O)O—, —NHC(═O)O—, —NHC(═O)NH—, —C(═S)NH—, —C(═O)S—, —NH—, O (oxygen), or S (sulfur), and wherein each m is independently an integer from 10 to 18 and wherein each n is independently an integer from 1 to 6; and
* indicates the point of attachment to the linker.

Embodiment 105. A dsRNAi agent of the structure of formula (II):

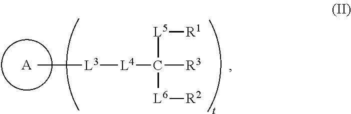

or a pharmaceutically acceptable salt thereof,
wherein A is defined according to any one of Embodiments 1 to 21, or
wherein A is a double stranded nucleic acid comprising a sense strand and an antisense strand,
the antisense strand forming a duplex with the sense strand, wherein each of the antisense strand and the sense strand is independently 17 to 25 nucleotides in length, wherein the nucleotide sequence of the antisense strand is at least 90% complementary to SEQ ID NO: 1, and the nucleotide sequence of the sense strand has no more than two mismatches to the nucleotide sequence of the antisense strand,
L³ is independently selected from:
   (i) a bond;

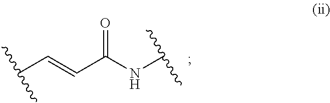 [SMILES: */C=C/C(=O)N*]

(iii) —OP(O)(OH)O—;
(iv) —OP(S)(OH)O—; and
(v) —O—;
L⁴ is independently selected from:
   (i) substituted or unsubstituted heteroalkylene;
   (ii) -L⁷-NH—C(O)— or -L⁷-C(O)—NH—, wherein L⁷ is substituted or unsubstituted alkylene;

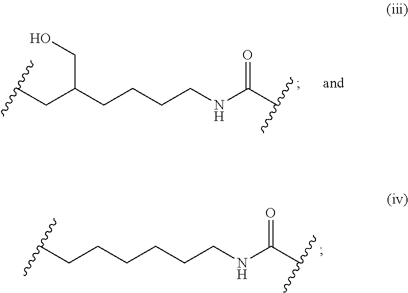 [SMILES: *CC(CO)CCCCNC(*)=O ; *CCCCCCNC(*)=O]

L⁵ is substituted or unsubstituted 2 to 20 membered heteroalkylene, or -L^{5A}L^{5B}, L^{5C}L^{5D}L^{5E}-,
   wherein L^{5A} is independently unsubstituted C₁-C₈ alkylene; L^{5B} is independently —NHC(O)—; L^{5C} is independently a bond; L^{D} is independently a bond or unsubstituted C₁-C₈ alkylene; and L^{5E} is independently a bond or —NHC(O)—;
L⁶ is —NHC(O)—;
R¹ and R² are independently unsubstituted Cn-C19 alkyl or unsubstituted Cn-C19 alkenyl;
R³ is hydrogen; and
t is 1.

Embodiment 106. A dsRNAi agent, or a pharmaceutically acceptable salt thereof, of formula

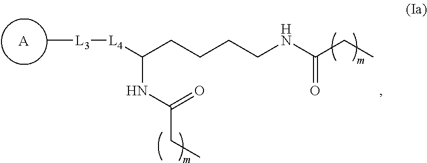

wherein:
each m, at each occurrence, is independently an integer from 10 to 18;
L³-L⁴- is defined according to any one of Embodiments 48 to 72 and 105;
and A is defined according to any one of Embodiments 1 to 21 and 105.

Embodiment 107. The dsRNAi agent of Embodiment 106, or a pharmaceutically acceptable salt thereof, of formula (Ib)

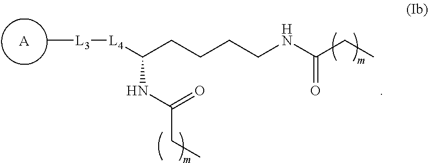

Embodiment 108. The dsRNAi agent of Embodiment 106, or a pharmaceutically acceptable salt thereof, of formula (Ic)

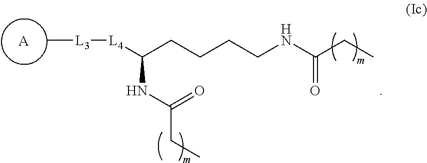

Embodiment 109. A dsRNAi agent, or a pharmaceutically acceptable salt thereof, of formula (Id)

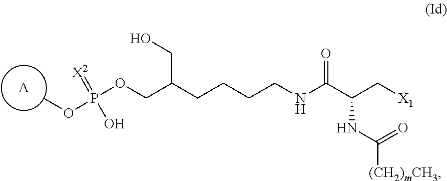

wherein
X² is selected from O and S;
X¹ is

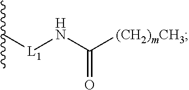

L¹ is —(CH₂)ₙ—, —(CH₂)ₙL₂(CH₂)ₙ—, or a bond;
L² is —C(═O)NH—, —C(═O)O—, —OC(═O)O—, —NHC(═O)O—, —NHC(═O)NH—, —C(═S)NH—, —C(═O)S—, —NH—, O (oxygen), or S (sulfur), and wherein each m is independently an integer from 10 to 18 and wherein each n is independently an integer from 1 to 6; and
A is defined according to any one of Embodiments 1 to 21 and 105.

Embodiment 110. The dsRNAi agent of Embodiment 104 or 109, or a pharmaceutically acceptable salt thereof, wherein each m is 10, L₁ is —(CH₂)ₙ—, and n is 3.

Embodiment 111. The dsRNAi agent of Embodiment 104 or 109, wherein each m is 11, L₁ is —(CH₂)ₙ—, and n is 3.

Embodiment 112. The dsRNAi agent of Embodiment 104 or 109, or a pharmaceutically acceptable salt thereof, wherein each m is 12, L₁ is —(CH₂)ₙ—, and n is 3.

Embodiment 113. The dsRNAi agent of Embodiment 104 or 109, or a pharmaceutically acceptable salt thereof, wherein each m is 13, L₁ is —(CH₂)ₙ—, and n is 3.

Embodiment 114. The dsRNAi agent of Embodiment 104 or 109, or a pharmaceutically acceptable salt thereof, wherein each m is 14, L₁ is —(CH₂)ₙ—, and n is 3.

Embodiment 115. The dsRNAi agent of Embodiment 104 or 109, or a pharmaceutically acceptable salt thereof, wherein each m is 15, L₁ is —(CH₂)ₙ—, and n is 3.

Embodiment 116. The dsRNAi agent of Embodiment 104 or 109, or a pharmaceutically acceptable salt thereof, wherein each m is 16, L₁ is —(CH₂)ₙ—, and n is 3.

Embodiment 117. The dsRNAi agent of Embodiment 104 or 109, or a pharmaceutically acceptable salt thereof, wherein each m is 17, L₁ is —(CH₂)ₙ—, and n is 3.

Embodiment 118. The dsRNAi agent of Embodiment 104 or 109, or a pharmaceutically acceptable salt thereof, wherein each m is 18, L₁ is —(CH₂)ₙ—, and n is 3.

Embodiment 119. The dsRNAi agent of Embodiment 104 or 109, or a pharmaceutically acceptable salt thereof, wherein each m is independently an integer from 12 to 16; and wherein each n is independently an integer from 1 to 6.

Embodiment 120. The dsRNAi agent of Embodiment 104 or 109, or a pharmaceutically acceptable salt thereof, wherein each m is independently an integer from 12 to 14; and wherein each n is independently an integer from 1 to 6.

Embodiment 121. The dsRNAi agent of Embodiment 104 or 109, or a pharmaceutically acceptable salt thereof, wherein L₁ is a bond; and each m is independently an integer from 12 to 16.

Embodiment 122. The dsRNAi agent of Embodiment 104 or 109, or a pharmaceutically acceptable salt thereof, wherein L₁ is —(CH₂)₃C(═O)NH(CH₂)₅—; and each m is independently an integer from 12 to 16.

Embodiment 123. The dsRNAi agent of any one of Embodiments 104 and 106 to 109, or a pharmaceutically acceptable salt thereof, wherein each m is 12.

Embodiment 124. The dsRNAi agent of any one of Embodiments 104 and 106 to 109, or a pharmaceutically acceptable salt thereof, wherein each m is 14.

Embodiment 125. The dsRNAi agent of any one of the preceding Embodiments, or a pharmaceutically acceptable salt thereof, wherein the nucleotide sequence of the antisense strand comprises at least 17, at least 18, at least 19, at least 20, or at least 21 contiguous nucleotides of a nucleotide sequence selected from any one of SEQ ID Nos: 294 to 584.

Embodiment 126. The dsRNAi agent of any one of the preceding Embodiments, or a pharmaceutically acceptable salt thereof, wherein the nucleotide sequence of the antisense strand comprises at least 19 contiguous nucleotides of a nucleotide sequence selected from any one of SEQ ID Nos: 294 to 584.

Embodiment 127. The dsRNAi agent of any one of the preceding Embodiments, or a pharmaceutically acceptable salt thereof, wherein the nucleotide sequence of the sense strand comprises at least 15, at least 16, at least 17, at least 18, or at least 19 contiguous nucleotides of a nucleotide sequence selected form any one of SEQ ID Nos: 3 to 293.

Embodiment 128. The dsRNAi agent of any one of the preceding Embodiments, or a pharmaceutically acceptable salt thereof, wherein the nucleotide sequence of the sense strand comprises at least 17 contiguous nucleotides of a nucleotide sequence selected form any one of SEQ ID Nos: 3 to 293.

Embodiment 129. The dsRNAi agent of any one of the preceding Embodiments, or a pharmaceutically acceptable salt thereof, wherein the antisense strand is 19 to 23 nucleotides in length.

Embodiment 130. The dsRNAi agent of any one of the preceding Embodiments, or a pharmaceutically acceptable salt thereof, wherein the antisense strand is 21 nucleotides in length.

Embodiment 131. The dsRNAi agent of any one of Embodiments 1 to 129, or a pharmaceutically acceptable salt thereof, wherein the antisense strand is 23 nucleotides in length.

Embodiment 132. The dsRNAi agent of any one of the preceding Embodiments, or a pharmaceutically acceptable salt thereof, wherein the sense strand is 19 to 23 nucleotides in length.

Embodiment 133. The dsRNAi agent of any one of the preceding Embodiments, or a pharmaceutically acceptable salt thereof, wherein the sense strand is 19 nucleotides in length.

Embodiment 134. The dsRNAi agent of any one of Embodiments 1 to 132, or a pharmaceutically acceptable salt thereof, wherein the sense strand is 21 nucleotides in length.

Embodiment 135. The dsRNAi agent of any one of the preceding Embodiments, or a pharmaceutically acceptable salt thereof, wherein the duplex is 19 to 23 nucleotide pairs in length.

Embodiment 136. The dsRNAi agent of any one of the preceding Embodiments, or a pharmaceutically acceptable salt thereof, wherein the duplex is 19 nucleotide pairs in length.

Embodiment 137. The dsRNAi agent of any one of Embodiments 1 to 135, or a pharmaceutically acceptable salt thereof, wherein the duplex is 21 nucleotide pairs in length.

Embodiment 138. The dsRNAi agent of any one of the preceding Embodiments, or a pharmaceutically acceptable salt thereof, wherein the nucleotide sequence of the antisense strand has no more than one mismatch to an equal length portion of SEQ ID NO: 1.

Embodiment 139. The dsRNAi agent of any one of the preceding Embodiments, or a pharmaceutically acceptable salt thereof, wherein the nucleotide sequence of the antisense strand has one mismatch to an equal length portion of SEQ ID NO: 1.

Embodiment 140. The dsRNAi agent of any one of Embodiments 1 to 138, or a pharmaceutically acceptable salt thereof, wherein the nucleotide sequence of the antisense strand has no mismatches to an equal length portion of SEQ ID NO: 1.

Embodiment 141. The dsRNAi agent of any one of Embodiments 138 to 140, or a pharmaceutically acceptable salt thereof, wherein the mismatch is at position 1 of the antisense strand, counting from the 5′ terminus of the antisense strand.

Embodiment 142. The dsRNAi agent of any one of the preceding Embodiments, or a pharmaceutically acceptable salt thereof, wherein the nucleotide sequence of the sense strand has no more than one mismatch to the nucleotide sequence of the antisense strand in the duplex.

Embodiment 143. The dsRNAi agent of any one of the preceding Embodiments, or a pharmaceutically acceptable salt thereof, wherein the nucleotide sequence of the sense strand has no mismatches to the nucleotide sequence of the antisense strand in the duplex.

Embodiment 144. The dsRNAi agent of any one of Embodiments 1 to 142, or a pharmaceutically acceptable salt thereof, wherein the nucleotide sequence of the sense strand has one mismatch to the nucleotide sequence of the antisense strand in the duplex.

Embodiment 145. The dsRNAi agent of Embodiment 144, or a pharmaceutically acceptable salt thereof, wherein the mismatch is at position 1 of the sense strand, counting from the 3′ terminus of the sense strand.

Embodiment 146. The dsRNAi agent of any one of the preceding Embodiments, or a pharmaceutically acceptable salt thereof, wherein the nucleotide sequence of the antisense strand is at least about 80%, at least about 85%, at least about 90%, at least about 91%, at least about 92%, at least about 93%, at least about 94%, at least about 95%, at least about 96%, at least about 97%, at least about 98%, or at least about 99% complementary to the nucleotide sequence of the sense strand in the duplex.

Embodiment 147. The dsRNAi agent of any one of the preceding Embodiments, or a pharmaceutically acceptable salt thereof, wherein the antisense strand is 21 nucleotides in length, the sense strand is 19 nucleotides in length, the duplex is 19 nucleotide pairs in length, and the nucleotide sequence of the antisense strand is at least about 94%, at least about 95%, at least about 96%, at least about 97%, at least about 98%, or at least about 99% complementary to the nucleotide sequence of the sense strand in the duplex.

Embodiment 148. The dsRNAi agent of any one of the preceding Embodiments, or a pharmaceutically acceptable salt thereof, wherein the antisense strand is 21 nucleotides in length, the sense strand is 19 nucleotides in length, the duplex is 19 nucleotide pairs in length, and the nucleotide sequence of the antisense strand is fully complementary to the nucleotide sequence of the sense strand in the duplex.

Embodiment 149. The dsRNAi agent of any one of the preceding Embodiments, or a pharmaceutically acceptable salt thereof, wherein the nucleotide sequence of the antisense strand is identical to a nucleotide sequence selected from any one of SEQ ID Nos: 294 to 584.

Embodiment 150. The dsRNAi agent of any one of the preceding Embodiments, or a pharmaceutically acceptable salt thereof, wherein the nucleotide sequence of the sense strand is identical to a nucleotide sequence selected from any one of SEQ ID Nos: 3 to 293.

Embodiment 151. The dsRNAi agent of any one of the preceding Embodiments, or a pharmaceutically acceptable salt thereof, wherein the antisense strand and the sense strand are not covalently linked.

Embodiment 152. The dsRNAi agent of any one of the preceding Embodiments, or a pharmaceutically acceptable salt thereof, wherein the antisense strand and the sense strand form at least one blunt end.

Embodiment 153. The dsRNAi agent of any one of the preceding Embodiments, or a pharmaceutically acceptable salt thereof, wherein the antisense strand and the sense strand form at least one nucleotide overhang of one to five nucleotides.

Embodiment 154. The dsRNAi agent of any one of the preceding Embodiments, or a pharmaceutically acceptable salt thereof, wherein the antisense strand comprises a 3′ nucleotide overhang.

Embodiment 155. The dsRNAi agent of Embodiment 154, or a pharmaceutically acceptable salt thereof, wherein the 3′ nucleotide overhang is two nucleotides in length.

Embodiment 156. The dsRNAi agent of Embodiment 154 or 155, wherein at least one nucleotide of the 3′ nucleotide overhang is a 2′-O-methylnucleotide.

Embodiment 157. The dsRNAi agent of any one of the preceding Embodiments, or a pharmaceutically acceptable salt thereof, wherein the double stranded nucleic acid comprises
(i) an antisense strand selected from SEQ ID Nos: 294 to 584, and
(ii) a sense strand selected from SEQ ID Nos: 3 to 293.

Embodiment 158. The dsRNAi agent of any one of the preceding Embodiments, or a pharmaceutically acceptable salt thereof, wherein the double stranded nucleic acid is modified.

Embodiment 159. The dsRNAi agent of any one of the preceding Embodiments, or a pharmaceutically acceptable salt thereof, wherein at least one nucleotide of the antisense strand is a modified nucleotide.

Embodiment 160. The dsRNAi agent of any one of the preceding Embodiments, or a pharmaceutically acceptable salt thereof, wherein at least one nucleotide of the sense strand is a modified nucleotide.

Embodiment 161. The dsRNAi agent of any one of the preceding Embodiments, or a pharmaceutically acceptable salt thereof, wherein each nucleotide of the antisense strand is a modified nucleotide.

Embodiment 162. The dsRNAi agent of any one of the preceding Embodiments, or a pharmaceutically acceptable salt thereof, wherein each nucleotide of the sense strand is a modified nucleotide.

Embodiment 163. The dsRNAi agent of any one of Embodiments 19 to 21 and 158 to 162, or a pharmaceutically acceptable salt thereof, wherein the modified nucleotide comprises one or more of a modified sugar moiety, a modified internucleoside linkage, and a 5′-terminal modified phosphate group.

Embodiment 164. The dsRNAi agent of Embodiment 163, or a pharmaceutically acceptable salt thereof, wherein the modified nucleotide comprising a modified sugar moiety is selected from a 2′-fluoro nucleotide, a 2′-O-methyl nucleotide, a 2′-O-methoxyethyl nucleotide, and a bicyclic sugar nucleotide.

Embodiment 165. The dsRNAi agent of Embodiment 163 or 164, or a pharmaceutically acceptable salt thereof, wherein the modified internucleoside linkage is a phosphorothioate internucleoside linkage.

Embodiment 166. The dsRNAi agent of any one of the preceding Embodiments, or a pharmaceutically acceptable salt thereof, wherein the first two internucleoside linkages at the 5′ terminus of the sense strand and the last two internucleoside linkages at the 3′ terminus of the sense strand are phosphorothioate internucleoside linkages.

Embodiment 167. The dsRNAi agent of any one of preceding Embodiments, or a pharmaceutically acceptable salt thereof, wherein the first two internucleoside linkages at the 5′ terminus of the antisense strand and the last two internucleoside linkages at the 3′ terminus of the antisense strand are phosphorothioate internucleoside linkages.

Embodiment 168. The dsRNAi agent of any one of Embodiments 163 to 167, or a pharmaceutically acceptable salt thereof, wherein the 5′-terminal modified phosphate group of the antisense strand is a 5′-(E)-vinylphosphonate.

Embodiment 169. The dsRNAi agent of any one of the preceding Embodiments, or a pharmaceutically acceptable salt thereof, wherein:
(i) the antisense strand is 21 nucleotides in length and wherein the nucleotides of the antisense strand are modified such that, counting from the 5′ terminus of the antisense strand, nucleotides 1, 3, 5, 7, 9, 11, 13, 15, 17, 19, 20, and 21 are 2′-O-methyl nucleotides, nucleotides 2, 4, 6, 8, 10, 12, 14, 16, 18 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and
(ii) the sense strand is 19 nucleotides in length and wherein the nucleotides of the sense strand are modified such that, counting from the 5′ terminus of the sense strand, nucleotides 1, 3, 5, 7, 9, 11, 13, 15, 17, and 19 are 2′ fluoro nucleotides and nucleotides 2, 4, 6, 8, 10, 12, 14, 16, and 18 are 2′-O-methyl nucleotides, the first two internucleoside linkages at the 5′ terminus and the last two internucleoside linkages at the 3′ terminus are phosphorothioate internucleoside linkages, and each other internucleotide is a phosphodiester internucleoside linkage.

Embodiment 170. The dsRNAi agent of any one of the preceding Embodiments, or a pharmaceutically acceptable salt thereof, wherein:
(i) the antisense strand is 21 nucleotides in length and the nucleotides of the antisense strand are modified such that, counting from the 5′ terminus of the antisense strand, nucleotides 1, 3, 5, 7, 9, 11, 13, 15, 17, 18, 19, 20, and 21 are 2′-O-methyl nucleotides, nucleotides 2, 4, 6, 8, 10, 12, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and
(ii) the sense strand is 19 nucleotides in length and wherein the nucleotides of the sense strand are modified such that, counting from the 5′ terminus of the sense strand, nucleotides 5, 7, 9, 11, 13, and 15 are 2′ fluoro nucleotides and nucleotides 1, 2, 3, 4, 6, 8, 10, 12, 14, 16, 17, 18, and 19 are 2′-O-methyl nucleotides, the first two internucleoside linkages at the 5′ terminus and the last two internucleoside linkages at the 3′ terminus are phosphorothioate internucleoside linkages, and each other internucleotide is a phosphodiester internucleoside linkage. In such embodiments, the antisense strand has the modification pattern of Pattern IV-1 and the sense strand has the modification pattern of Pattern XXV.

Embodiment 171. The dsRNAi agent of any one of the preceding Embodiments, or a pharmaceutically acceptable salt thereof, wherein:
(i) the antisense strand is 21 nucleotides in length and the nucleotides of the antisense strand are modified such that, counting from the 5′ terminus of the antisense strand, nucleotides 1, 3, 4, 5, 7, 8, 9, 11, 13, 15, 17, 18, 19, 20, and 21 are 2′-O-methyl nucleotides, nucleotides 2, 6, 10, 12, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and
(ii) the sense strand is 19 nucleotides in length and the nucleotides of the sense strand are modified such that, counting from the 5′ terminus of the sense strand, nucleotides 5, 8, 9, 11, and 13 are 2′ fluoro nucleotides and nucleotides 1, 2, 3, 4, 6, 7, 10, 12, 14, 15, 16, 17, 18, and 19 are 2′-O-methyl nucleotides, the first two internucleoside linkages at the 5′ terminus and the last two internucleoside linkages at the 3′ terminus are phosphorothioate internucleoside linkages, and each other internucleotide is a phosphodiester internucleoside linkage.

Embodiment 172. The dsRNAi agent of any one of the preceding Embodiments, or a pharmaceutically acceptable salt thereof, wherein:
(i) antisense strand is 21 nucleotides in length and the nucleotides of the antisense strand are modified such that, counting from the 5′ terminus of the antisense strand, nucleotides 1, 3, 4, 5, 7, 8, 9, 10, 11, 12, 13, 15, 17, 18, 19, 20, and 21 are 2′-O-methyl nucleotides, nucleotides 2, 6, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkages are phosphodiester internucleoside linkages; and
(ii) the sense strand is 19 nucleotides in length and the nucleotides of the sense strand are modified such that, counting from the 5′ terminus of the sense strand, nucleotides 5, 7, 8, and 9 are 2′-fluoro nucleotides and nucleotides 1, 2, 3, 4, 6, 10, 11, 12, 13, 14, 15, 16, 17, 18, and 19 are 2′-O-methyl nucleotides, the first two internucleoside linkages at the 5′ terminus and the last two internucleoside linkages at the 3′ terminus are phosphorothioate internucleoside linkages, and all other internucleoside linkages are phosphodiester internucleoside linkages.

Embodiment 173. The dsRNAi agent of any one of the preceding Embodiments, or a pharmaceutically acceptable salt thereof, wherein the dsRNAi agent is selected from a modified siRNA in Table 8, Table 9 or Table 11.

Embodiment 174. The dsRNAi agent of Embodiment 1, or a pharmaceutically acceptable salt thereof, wherein the dsRNAi agent is DT-001068.

Embodiment 175. The dsRNAi agent of Embodiment 1, or a pharmaceutically acceptable salt thereof, wherein the dsRNAi agent is DT-002211.

Embodiment 176. The dsRNAi agent of Embodiment 1, or a pharmaceutically acceptable salt thereof, wherein the dsRNAi agent is DT-003035.

Embodiment 177. The dsRNAi agent of Embodiment 1, or a pharmaceutically acceptable salt thereof, wherein the dsRNAi agent is DT-003036.

Embodiment 178. The dsRNAi agent of Embodiment 1, or a pharmaceutically acceptable salt thereof, wherein the dsRNAi agent is DT-003038.

Embodiment 179. The dsRNAi agent of any one of the preceding Embodiments, wherein the pharmaceutical salt is a sodium salt.

Embodiment 180. A dsRNAi agent comprising:
(i) an antisense strand having a nucleotide sequence of SEQ ID NO: 1268; and
(ii) a sense stand strand having a nucleotide sequence of SEQ ID NO: 1267.

Embodiment 181. A dsRNAi agent comprising:
(i) an antisense strand having a nucleotide sequence of SEQ ID NO: 1312; and
(ii) a sense stand strand having a nucleotide sequence of SEQ ID NO: 1311.

Embodiment 182. A dsRNAi agent comprising:
(i) an antisense strand having a nucleotide sequence of SEQ ID NO: 1346; and
(ii) a sense stand strand having a nucleotide sequence of SEQ ID NO: 1345.

Embodiment 183. A dsRNAi agent comprising:
(i) an antisense strand having a nucleotide sequence of SEQ ID NO: 1348; and
(ii) a sense stand strand having a nucleotide sequence of SEQ ID NO: 1347.

Embodiment 184. A dsRNAi agent comprising:
(i) an antisense strand having a nucleotide sequence of SEQ ID NO: 1352; and
(ii) a sense stand strand having a nucleotide sequence of SEQ ID NO: 1351.

Embodiment 185. A dsRNAi agent, or a pharmaceutically acceptable salt thereof, comprising:
(i) an antisense strand having a nucleotide sequence of

(SEQ ID NO: 1352)
5′-X033U1027p001C007p001A004p000A004p000U004p000
A007p000A004p000A004p000U004p000A004p000U004p000
C004p000C004p000A007p000A004p000A007p000C004p000
C004p000G004p001C004p001C004-3′;

and
(ii) a sense stand strand having a nucleotide sequence of

(SEQ ID NO: 1351)
5′-C004p001G004p001G004p000U004p000U007p000U004
p000G007p000G007p000A007p000U004p000A004p000U004
p000U004p000U004p000A004p000U004p000U004p001G004
p001U004p000X2140-3′,

wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

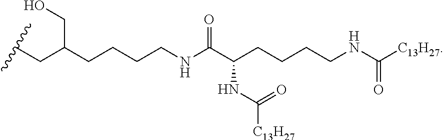

Embodiment 186. A pharmaceutical composition comprising a dsRNAi agent of any one of the preceding Embodiments, or a pharmaceutically acceptable salt thereof, and a pharmaceutically acceptable carrier.

Embodiment 187. The pharmaceutical composition of Embodiment 186, wherein the pharmaceutically acceptable carrier is a sterile aqueous solution.

Embodiment 188. The pharmaceutical composition of Embodiment 187, wherein the sterile aqueous solution is a sterile saline solution.

Embodiment 189. A method for inhibiting the expression of DMPK mRNA in a cell, the method comprising contacting a dsRNAi agent of any one of Embodiments 1 to 185, or a pharmaceutically acceptable salt thereof, with the cell.

Embodiment 190. A method for reducing or preventing aggregation of one or more DMPK mRNA transcripts within a cell, the method comprising contacting a dsRNAi agent of any one of Embodiments 1 to 185, or a pharmaceutically acceptable salt thereof, with the cell.

Embodiment 191. The method of Embodiment 189 or 190, wherein the cell is a muscle cell, e.g., a skeletal muscle cell.

Embodiment 192. A method for inhibiting the expression of DMPK in a subject in need thereof, the method comprising administering to the subject a therapeutically effective amount of a dsRNAi agent of any one of Embodiments 1 to 185, or a pharmaceutically acceptable salt thereof, or a pharmaceutical composition according to any one of Embodiments 186 to 188.

Embodiment 193. A method for treating myotonic dystrophy type I (DM1) in a subject in need thereof, the method comprising administering to the subject a therapeutically effective amount of a dsRNAi agent of any one of Embodiments 1 to 185, or a pharmaceutically acceptable salt thereof, or a pharmaceutical composition according to any one of Embodiments 186 to 188.

Embodiment 194. A method for delaying the onset of DM1 in a subject in need thereof, the method comprising administering to the subject a therapeutically effective amount of a dsRNAi agent of any one of Embodiments 1 to 185, or a pharmaceutically acceptable salt thereof, or a pharmaceutical composition according to any one of Embodiments 186 to 188.

Embodiment 195. A method for treating DM1 in a subject in need thereof, the method comprising administering to the subject a therapeutically effective amount of a dsRNAi agent of any one of Embodiments 1 to 185, or a pharmaceutically acceptable salt thereof, or a pharmaceutical composition according to any one of Embodiments 186 to 188, wherein the administration of the dsRNAi agent relieves at least one symptom of DM1 in at least one muscle.

Embodiment 196. A method for treating DM1 in a subject in need thereof, the method comprising systemically administering to the subject a therapeutically effective amount of a dsRNAi agent of any one of Embodiments 1 to 185, or a pharmaceutically acceptable salt thereof, or a pharmaceutical composition according to any one of Embodiments 186 to 188, wherein the administration of the dsRNAi agent relieves at least one symptom of DM1 in at least one muscle.

Embodiment 197. The method of Embodiment 195 or 196, wherein the at least one symptom of DM1 is myotonia.

Embodiment 198. The method of any one of Embodiments 195 to 197, wherein the at least one symptom of DM1 is aggregation of musclebind-like-1 (MBNL-1) protein in ribonuclear foci within myonuclei.

Embodiment 199. The method of any one of Embodiments 195 to 198, wherein the at least one symptom of DM1 is abnormal splicing of at least one RNA transcript in muscle cells.

Embodiment 200. The method of Embodiment 199, wherein the at least one RNA transcript is selected from the group consisting of Serca-1, m-Titin, Zasp, and CIC-1.

Embodiment 201. The method of any one of Embodiments 191 and 195 to 200, wherein the muscle is selected from the group consisting of skeletal muscle, smooth muscle, and cardiac muscle.

Embodiment 202. The method of Embodiment 201, wherein the muscle is selected from the group consisting of semitendinosus, platysma, gluteus, rectus femoris, triceps, biceps, quadriceps, soleus, EDL (extensor digitorum longus), diaphragm, tibialis anterior, gastrocnemius, heart, trapezius, diaphragm and tongue.

Embodiment 203. The method of any one of Embodiments 189 to 202, wherein DMPK gene expression is inhibited by at least about 40%, at least about 50%, at least about 60%, at least about 70%, at least about 80%, or at least about 90%, relative to a control, e.g., when measured by RT-PCR, e.g., RT-qPCR.

Embodiment 204. The method of any one of Embodiments 189 to 203, wherein the administration is intravenous administration or subcutaneous administration.

Embodiment 205. A dsRNAi agent according to any one of claims 1 to 185, or a pharmaceutically acceptable salt thereof, for use in a method for inhibiting the expression of DMPK mRNA in a cell, the method comprising contacting the dsRNAi agent, or a pharmaceutically acceptable salt thereof, with the cell.

Embodiment 206. A dsRNAi agent according to any one of Embodiments 1 to 185, or a pharmaceutically acceptable salt thereof, for use in a method for reducing or preventing aggregation of one or more DMPK mRNA transcripts within a cell, the method comprising contacting the dsRNAi agent, or a pharmaceutically acceptable salt thereof, with the cell.

Embodiment 207. The dsRNAi agent, or a pharmaceutically acceptable salt thereof, for use according to Embodiment 205 or 206, wherein the cell is a muscle cell, e.g., a skeletal muscle cell.

Embodiment 208. A dsRNAi agent according to any one of Embodiments 1 to 185, or a pharmaceutically acceptable salt thereof, or a pharmaceutical composition according to any one of Embodiments 186 to 188, for use in a method for inhibiting the expression of DMPK in a subject in need thereof, the method comprising administering to the subject a therapeutically effective amount of the dsRNAi agent, a or pharmaceutically acceptable salt thereof, or the pharmaceutical composition.

Embodiment 209. A dsRNAi agent according to any one of Embodiments 1 to 185, or a pharmaceutically acceptable salt thereof, or a pharmaceutical composition according to any one of Embodiments 186 to 188, for use in the treatment of myotonic dystrophy type I (DM1).

Embodiment 210. A dsRNAi agent according to any one of Embodiments 1 to 185, or a pharmaceutically acceptable salt thereof, or a pharmaceutical composition according to any one of Embodiments 186 to 188, for use in a method for delaying the onset of DM1 in a subject in need thereof, the method comprising administering to the subject a therapeutically effective amount of the dsRNAi agent, or a pharmaceutically acceptable salt thereof, or the pharmaceutical composition.

Embodiment 211. A dsRNAi agent according to any one of Embodiments 1 to 185, or a pharmaceutically acceptable salt thereof, or a pharmaceutical composition according to any one of Embodiments 186 to 188, for use in the treatment of DM1, wherein the dsRNAi agent or pharmaceutical composition relieves at least one symptom of DM1 in at least one muscle.

Embodiment 212. A dsRNAi agent according to any one of Embodiments 1 to 185, or a pharmaceutically acceptable salt thereof, or a pharmaceutical composition according to any one of Embodiments 186 to 188, for use in the treatment of DM1, wherein the dsRNAi agent or pharmaceutical composition is administered systemically, and wherein the dsRNAi agent or pharmaceutical composition relieves at least one symptom of DM1 in at least one muscle.

Embodiment 213. The dsRNAi agent, or a pharmaceutically acceptable salt thereof, or the pharmaceutical composition, for use according to Embodiment 211 or 212, wherein the at least one symptom of DM1 is myotonia.

Embodiment 214. The dsRNAi agent, or a pharmaceutically acceptable salt thereof, or the pharmaceutical composition, for use according to any one of Embodiments 211 to 213, wherein the at least one symptom of DM1 is aggregation of musclebind-like-1 (MBNL-1) protein in ribonuclear foci within myonuclei.

Embodiment 215. The dsRNAi agent, or a pharmaceutically acceptable salt thereof, or the pharmaceutical composition, for use according to any one of Embodiments 211 to 214, wherein the at least one symptom of DM1 is abnormal splicing of at least one RNA transcript in muscle cells.

Embodiment 216. The dsRNAi agent, or a pharmaceutically acceptable salt thereof, or the pharmaceutical composition, for use according to Embodiment 215, wherein the at least one RNA transcript is selected from the group consisting of Serca-1, m-Titin, Zasp, and CIC-1.

Embodiment 217. The dsRNAi agent, or a pharmaceutically acceptable salt thereof, or the pharmaceutical composition, for use according to any one of Embodiments 207 and 211 to 216, wherein the muscle is selected from the group consisting of skeletal muscle, smooth muscle, and cardiac muscle.

Embodiment 218. The dsRNAi agent, or a pharmaceutically acceptable salt thereof, or the pharmaceutical composition, for use according to Embodiment 217, wherein the muscle is selected from the group consisting of semitendinosus, platysma, gluteus, rectus femoris, triceps, biceps, quadriceps, soleus, EDL (extensor digitorum longus), diaphragm, tibialis anterior, gastrocnemius, heart, trapezius, diaphragm and tongue.

Embodiment 219. The dsRNAi agent, or a pharmaceutically acceptable salt thereof, or the pharmaceutical composition, for use according to any one of Embodiments 205 to 218, wherein DMPK gene expression is inhibited by at least about 40%, at least about 50%, at least about 60%, at least about 70%, at least about 80%, or at least about 90%, relative to a control, e.g., when measured by RT-PCR, e.g., RT-qPCR.

Embodiment 220. The dsRNAi agent, or a pharmaceutically acceptable salt thereof, or the pharmaceutical composition, for use according to any one of Embodiments 205 to 219, wherein the dsRNAi agent or pharmaceutical composition, is administered intravenously or subcutaneously.

Embodiment 221. Use of a dsRNAi agent according to any one of Embodiments 1 to 185, or a pharmaceutically acceptable salt thereof, or a pharmaceutical composition according to any one of Embodiments 186 to 188, in a method according to any one of Embodiments 189 to 204.

Embodiment 222. Use of a dsRNAi agent according to any one of Embodiments 1 to 185, or a pharmaceutically acceptable salt thereof, or a pharmaceutical composition according to any one of Embodiments 186 to 188, in the manufacture of a medicament for the treatment of DM1.

### RNAi Agents

In an aspect, the disclosure provides an RNAi agent including a double stranded RNA (dsRNA). In an aspect, also provided is a dsRNA interference (dsRNAi) agent that includes a double stranded nucleic acid comprising (i) a sense strand and (ii) an antisense strand, and a ligand attached to at least one of the sense strand and the antisense strand.

In certain aspects, the double stranded nucleic acids act through the RNA interference pathway to inhibit the expression of the DMPK mRNA. In some aspects, the antisense strand and sense strand of a compound are two separate strands and are not covalently linked and form a small interfering RNA (siRNA). In some aspects, the antisense strand and sense strand of a compound are covalently linked by a nucleotide linker to form a short hairpin RNA (shRNA).

A dsRNA is a complex of ribonucleic acid (RNA) molecules formed in a duplex structure. In certain aspect, the dsRNA is a short interfering RNA (siRNA) that has 10 to 30, or particularly 17-25 nucleotides, more particularly 19-23 nucleotides, in each RNA molecule, respectively, “passenger strand” and “guide strand”, and can be incorporated into an RNA-induced silencing complex (RISC). The siRNA is dissociated or unwounded in the RISC, and the passenger strand is degraded while the guide strand remains in the RISC pathway. The guide strand can subsequently bind to a mRNA molecule that includes a complementary sequence to the guide strand and induce or initiate cleavage or degradation of the mRNA molecule. In a certain aspect, the mRNA encodes a DMPK target gene, e.g., mRNA transcript of said target gene, such that expression of the DMPK target gene is suppressed or inhibited through a post-transcriptional gene-silencing (“RNA silencing”). A guide RNA molecule has a complementary sequence to a target mRNA sequence and has anti-parallel orientation to the target gene, so it is interchangeably referred to as an antisense strand. A passenger RNA molecule forming a duplex with the guide RNA and having a complementary sequence to the guide strand (antisense strand) has the same orientation with the target mRNA sequence, so it is interchangeably referred to as a sense strand.

In another aspect, the disclosure provides a dsRNAi agent, or a pharmaceutically acceptable salt thereof, of formula (I)

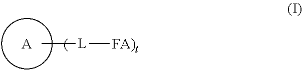

wherein:
A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
L is a linker;
FA is a fatty acid moiety capable of facilitating delivery of the double stranded nucleic acid into a cell, e.g., skeletal muscle cell; and
t is an integer from 1 to 5.

Embodiments of the present disclosure relate to dsRNAi agents comprising double stranded nucleic acids targeted to the human DMPK (hDMPK) mRNA, e.g., NCBI Reference Sequence NM_001081563.2, deposited with GenBank on Jan. 4, 2014; SEQ ID NO: 1. The double stranded RNAi agent of a compound of formula (I), includes an antisense strand complementary to the DMPK mRNA and a sense strand complementary to the antisense strand.

The dsRNAi agents disclosed herein, including those of formula (I) and subformulae thereof, upon delivery to a cell expressing a DMPK gene, inhibit or knockdown expression of one or more DMPK genes in vivo and/or in vitro.

In some embodiments, the dsRNAi agents of the present disclosure are capable of inhibiting DMPK gene expression by at least about 40%, at least about 50%, at least about 60%, at least about 70%, at least about 80%, or at least about 90% in comparison to a control, in particular, when measured by RT-PCR, e.g., RT-qPCR.

Further embodiments of the double stranded nucleic acid A, linker L and fatty acid moiety FA are described in the following sections.

### DMPK Double-Stranded Nucleic Acid

The DMPK double stranded nucleic acid sense and antisense strands anneal to form a duplex. The sense strand and an antisense strand of a DMPK double stranded nucleic acid can be partially, substantially, or fully complementary to each other. Within the complementary duplex region, the sense strand is at least 80% complementary to the antisense strand, as measured over the entirety of the sense strand. In some embodiments, the sense strand is at least about 85%, at least about 90%, at least about 91%, at least about 92%, at least about 93%, at least about 94%, at least about 95%, at least about 96%, at least about 97%, at least about 98%, or at least about 99% complementary to the antisense strand, as measured over the entirety of the sense strand. In some embodiments, the sense strand is at least about 95% complementary to the antisense strand, as measured over the entirety of the sense strand. In some embodiments, the sense strand is 100% complementary to the antisense strand, as measured over the entirety of the sense strand.

Provided below are features of the double stranded nucleic acids of the disclosure, including those of formula (I) and subformulae thereof, such as length, nucleotide sequence, and nucleotide modifications. It is understood that an embodiment of a sense strand may apply to a sense strand of any double stranded nucleic acid provided herein, including siRNAs and shRNAs, preferably siRNA.

In certain aspects, the double stranded nucleic acid includes a sense strand and an antisense strand forming a duplex with the sense strand, wherein each of the antisense strand and the sense strand is independently 10 to 30 nucleotides in length.

In certain aspects, the double stranded nucleic acid includes an antisense strand and a sense strand hybridized to form a double stranded nucleic acid, wherein each of the antisense strand and sense strands is 17 to 25 nucleotides in length, wherein the nucleotide sequence of the antisense strand is at least 90% complementary to the human DMPK mRNA (e.g., NCBI Reference Sequence NM_001081563.2, deposited with GenBank on Jan. 4, 2014; SEQ ID NO: 1), and the nucleotide sequence of the sense strand has no more than two mismatches to the nucleotide sequence of the antisense strand in the double-stranded region.

The nucleic acid sequence of SEQ ID NO: 1 is indicated below.

| | |
|---|---|
| 1 | gccacaagcc tccaccccag ctggtccccc acccaggctg cccagtttaa cattcctagt |
| 61 | cataggacct tgacttctga gaggcctgat tgtcatctgt aaataagggg taggactaaa |
| 121 | gcactcctcc tggaggactg agagatgggc tggaccggag cacttgagtc tgggatatgt |
| 181 | gaccatgcta cctttgtctc cctgtcctgt tccttccccc agccccaaat ccagggtttt |
| 241 | ccaaagtgtg gttcaagaac cacctgcatc tgaatctaga ggtactggat acaaccccac |
| 301 | gtctgggccg ttacccagga cattctacat gagaacgtgg gggtggggcc ctggctgcac |
| 361 | ctgaactgtc acctggagtc agggtggaag gtggaagaac tgggtcttat ttccttctcc |
| 421 | ccttgttctt tagggtctgt ccttctgcag actccgttac cccaccctaa ccatcctgca |
| 481 | cacccttgga gccctctggg ccaatgccct gtcccgcaaa gggcttctca ggcatctcac |
| 541 | ctctatggga gggcattttt ggcccccaga accttacacg gtgtttatgt ggggaagccc |
| 601 | ctgggaagca gacagtccta gggtgaagct gagaggcaga gagaagggga gacagacaga |
| 661 | gggtggggct ttcccccttg tctccagtgc cctttctggt gaccctcggt tcttttcccc |
| 721 | caccaccccc ccagcggagc ccatcgtggt gaggcttaag gaggtccgac tgcagaggga |
| 781 | cgacttcgag attctgaagg tgatcggacg cggggcgttc agcgaggtag cggtagtgaa |
| 841 | gatgaagcag acgggccagg tgtatgccat gaagatcatg aacaagtggg acatgctgaa |
| 901 | gaggggcgag gtgtcgtgct tccgtgagga gagggacgtg ttggtgaatg gggaccggcg |
| 961 | gtggatcacg cagctgcact tcgccttcca ggatgagaac tacctgtacc tggtcatgga |
| 1021 | gtattacgtg ggcggggacc tgctgacact gctgagcaag tttggggagc ggattccggc |
| 1081 | cgagatggcg cgcttctacc tggcggagat tgtcatggcc atagactcgg tgcaccggct |
| 1141 | tggctacgtg cacagggaca tcaaacccga caacatcctg ctggaccgct gtggccacat |
| 1201 | ccgcctggcc gacttcggct cttgcctcaa gctgcgggca gatggaacgg tgcggtcgct |
| 1261 | ggtggctgtg ggcaccccag actacctgtc ccccgagatc ctgcaggctg tgggcggtgg |
| 1321 | gcctgggaca ggcagctacg ggcccgagtg tgactggtgg gcgctgggtg tattcgccta |
| 1381 | tgaaatgttc tatgggcaga cgcccttcta cgcggattcc acggcggaga cctatggcaa |
| 1441 | gatcgtccac tacaaggagc acctctctct gccgctggtg gacgaagggg tccctgagga |
| 1501 | ggctcgagac ttcattcagc ggttgctgtg tcccccggag acacggctgg gccggggtgg |
| 1561 | agcaggcgac ttccggacac atcccttctt ctttggcctc gactgggatg gtctccggga |
| 1621 | cagcgtgccc ccctttacac cggatttcga aggtgccacc gacacatgca acttcgactt |
| 1681 | ggtggaggac gggctcactg ccatggtgag cgggggcggg gagacactgt cggacattct |
| 1741 | ggaaggtgcg ccgctagggg tccacctgcc ttttgtgggc tactcctact cctgcatggc |
| 1801 | cctcagggac agtgaggtcc caggccccac acccatggaa ctggaggccg agcagctgct |
| 1861 | tgagccacac gtgcaagcgc ccagcctgga gccctcggtg tccccacagg atgaaacagc |
| 1921 | tgaagtggca gttccagcgg ctgtccctgc ggcagaggct gaggccgagg tgacgctgcg |
| 1981 | ggagctccag gaagccctgg aggaggaggt gctcacccgg cagagcctga gccgggagat |
| 2041 | ggaggccatc cgcacggaca accagaactt cgccagtcaa ctacgcgagg cagaggctcg |
| 2101 | gaaccgggac ctagaggcac acgtccggca gttgcaggag cggatggagt tgctgcaggc |
| 2161 | agagggagcc acagctgtca cgggggtccc cagtccccgg gccacggatc caccttccca |
| 2221 | tctagatggc cccccggccg tggctgtggg ccagtgcccg ctggtggggc caggccccat |
| 2281 | gcaccgccgc cacctgctgc tccctgccag ggtccctagg cctggcctat cggaggcgct |
| 2341 | ttccctgctc ctgttcgccg ttgttctgtc tcgtgccgcc gccctgggct gcattgggtt |
| 2401 | ggtggcccac gccggccaac tcaccgcagt ctggcgccgc ccaggagccg cccgcgctcc |
| 2461 | ctgaacccta gaactgtctt cgactccggg gccccgttgg aagactgagt gcccggggca |
| 2521 | cggcacagaa gccgcgccca ccgcctgcca gttcacaacc gctccgagcg tgggtctccg |
| 2581 | cccagctcca gtcctgtgat ccgggcccgc cccctagcgg ccggggaggg aggggccggg |
| 2641 | tccgcggccg gcgaacgggg ctcgaagggt ccttgtagcc gggaatgctg ctgctgctgc |
| 2701 | tgctgctgct gctgctgctg ctgctgctgc tgctgctgct gctgctgggg ggatcacaga |
| 2761 | ccatttcttt ctttcggcca ggctgaggcc ctgacgtgga tgggcaaact gcaggcctgg |
| 2821 | gaaggcagca agccgggccg tccgtgttcc atcctccacg cacccccacc tatcgttggt |
| 2881 | tcgcaaagtg caaagctttc ttgtgcatga cgccctgctc tggggagcgt ctggcgcgat |
| 2941 | ctctgcctgc ttactcggga aatttgcttt tgccaaaccc gctttttcgg ggatcccgcg |
| 3001 | cccccctcct cacttgcgct gctctcggag ccccagccgg ctccgcccgc ttcggcggtt |
| 3061 | tggatattta ttgacctcgt cctccgactc gctgacaggc tacaggaccc ccaacaaccc |
| 3121 | caatccacgt tttggatgca ctgagacccc gacattcctc ggtatttatt gtctgtcccc |
| 3181 | acctaggacc cccacccccg accctcgcga ataaaaggcc ctccatctgc ccaaagctct |
| 3241 | |

In certain aspects, the double stranded nucleic acid includes an antisense strand and a sense strand hybridized to form a double stranded nucleic acid, wherein each of the antisense strand and sense strands is 17 to 25 nucleotides in length, wherein the nucleotide sequence of the antisense strand is at least 90% complementary to the human DMPK mRNA (e.g., NCBI Reference Sequence NM_001081563.2, deposited with GenBank on Jan. 4, 2014; SEQ ID NO: 1), and the nucleotide sequence of the sense strand has no more than one mismatch to the nucleotide sequence of the antisense strand in the double-stranded region.

In some embodiments, the sense strand comprises a nucleic acid sequence that is at least 80% identical to SEQ ID NO: 1, as measured over the entirety of the sense strand. In some embodiments, the sense strand comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO: 1, as measured over the entirety of the sense strand. In some embodiments, the sense strand comprises a nucleic acid sequence that is 100% identical to SEQ ID NO: 1, as measured over the entirety of the sense strand.

In some embodiments, the double stranded nucleic acid includes an antisense strand and a sense strand hybridized to form a duplex structure, wherein each of the antisense strand and sense strands is 17 to 25 nucleotides in length.

In some embodiments, the sense strand is 17 to 25 nucleotides in length and comprises a nucleic acid sequence that is at least about 94%, at least about 95%, at least about 96%, at least about 97%, at least about 98%, at least about 99%, or is 100% identical to SEQ ID NO: 1, as measured over the entirety of the sense strand.

In some embodiments, the sense strand comprises a region of sequence identity to an mRNA encoding DMPK, and wherein the region of sequence identity comprises at least 8 contiguous nucleotides of SEQ ID NO: 1. In some embodiments, the region of sequence identity comprises least 9, at least 10, at least 11, at least 12, at least 13, at least 14, at least 15, at least 16, at least 17, at least 18, at least 19, or at least 20 contiguous nucleotides of SEQ ID NO: 1. In some embodiments, the sense strand comprises a region of sequence identity to an mRNA encoding DMPK, and wherein the region of sequence identity comprises at least 8, at least 9, at least 10, at least 11, at least 12, at least 13, at least 14, at least 15, at least 16, at least 17, at least 18, at least 19, or at least 20 contiguous nucleotides of SEQ ID NO: 1, that is at least about 80%, at least about 85%, at least about 90%, at least about 91%, at least about 92%, at least about 93%, at least about 94%, at least about 95%, at least about 96%, at least about 97%, at least about 98%, at least about 99%, or is 100% identical to SEQ ID NO: 1. In some embodiments, the sense strand comprises a region of sequence identity to an mRNA encoding DMPK, and wherein the region of sequence identity comprises 19 contiguous nucleotides of SEQ ID NO: 1, that is at least about 95% identical to SEQ ID NO: 1.

In some embodiments, the antisense strand comprises a nucleic acid sequence that is at least 80% complementary to a region of equal length of SEQ ID NO: 1, as measured over the entirety of the antisense strand. In some embodiments, the antisense strand comprises a nucleic acid sequence that is at least about 80%, at least about 85%, at least about 90%, at least about 91%, at least about 92%, at least about 93%, at least about 94%, at least about 95%, at least about 96%, at least about 97%, at least about 98%, at least about 99% complementary to a region of equal length of SEQ ID NO: 1, as measured over the entirety of the antisense strand. In some embodiments, the antisense strand comprises a nucleic acid sequence that is 100% complementary to a region of equal length of SEQ ID NO: 1, as measured over the entirety of the antisense strand. In some embodiments, the antisense strand includes a sequence of at least 16, at least 17, at least 18, at least 19, at least 20, at least 21, at least 22, at least 23 nucleotides, that is at least about 85%, at least about 90%, at least about 91%, at least about 92%, at least about 93%, at least about 94%, at least about 95%, at least about 96%, at least about 97%, at least about 98%, at least about 99%, or is 100% complementary to a region of equal length of SEQ ID NO: 1 (i.e., the antisense strand has a region of at least 16, at least 17, at least 18, at least 19, at least 20, at least 21, at least 22, at least 23 nucleotides that is at least about 85%, at least about 90%, at least about 91%, at least about 92%, at least about 93%, at least about 94%, at least about 95%, at least about 96%, at least about 97%, at least about 98%, or at least about 99% base paired or is 100% base paired). In some embodiments, the antisense strand comprises a sequence of 21 nucleotides that is 100% complementary to a region of equal length of SEQ ID NO: 1.

In some embodiments, the antisense strand is 15 to 25 nucleotides in length. In some embodiments, the antisense strand is 17 to 23 nucleotides in length. In some embodiments, the antisense strand is 19 to 23 nucleotides in length. In some embodiments, the antisense strand is 19 to 21 nucleotides in length. In some embodiments, the antisense strand is 21 to 23 nucleotides in length. In some embodiments, the antisense strand is 15 nucleotides in length. In some embodiments, the antisense strand is 16 nucleotides in length. In some embodiments, the antisense strand is 17 nucleotides in length. In some embodiments, the antisense strand is 18 nucleotides in length. In some embodiments, the antisense strand is 19 nucleotides in length. In some embodiments, the antisense strand is 20 nucleotides in length. In some embodiments, the antisense strand is 21 nucleotides in length. In some embodiments, the antisense strand is 22 nucleotides in length. In some embodiments, the antisense strand is 23 nucleotides in length. In some embodiments, the antisense strand is 24 nucleotides in length. In some embodiments, the antisense strand is 25 nucleotides in length.

In some embodiments, the nucleotide sequence of the antisense strand can be selected by selecting a target region and length in the DMPK mRNA. In some embodiments, a dsRNAi as described herein may target a nucleotide region selected from nucleotides 3001 to 3181 of SEQ ID NO: 1 that is at least about 85%, at least about 90%, at least about 91%, at least about 92%, at least about 93%, at least about 94%, at least about 95%, at least about 96%, at least about 97%, at least about 98%, at least about 99%, or is 100% complementary to the nucleotide region of SEQ ID NO: 1.

In some embodiments, the nucleotide sequence of the antisense strand is at least about 95% complementary to SEQ ID NO: 1. In some embodiments, the nucleotide sequence of the antisense strand is 100% complementary to SEQ ID NO: 1. In some embodiments, the nucleotide sequence of the antisense strand is at least about 85%, at least about 90%, at least about 91%, at least about 92%, at least about 93%, at least about 94%, at least about 95%, at least about 96%, at least about 97%, at least about 98%, at least about 99%, or is 100% complementary to nucleotides 3054 to 3074 of SEQ ID NO: 1. In some embodiments, the nucleotide sequence of the antisense strand is at least about 85%, at least about 90%, at least about 91%, at least about 92%, at least about 93%, at least about 94%, at least about 95%, at least about 96%, at least about 97%, at least about 98%, at least about 99%, or is 100% complementary to nucleotides 3155 to 3175 of SEQ ID NO: 1. In some embodiments, the nucleotide sequence of the antisense strand is 100% complementary to nucleotides 3054 to 3074 of SEQ ID NO: 1. In some embodiments, the nucleotide sequence of the antisense strand is about 95% complementary to nucleotides 3155 to 3175 of SEQ ID NO: 1.

In some embodiments, the nucleotide sequence of the antisense strand has no more than one mismatch to an equal length portion of SEQ ID NO: 1. In some embodiments, the nucleotide sequence of the antisense strand has one mismatch to an equal length portion of SEQ ID NO: 1. In some embodiments, the nucleotide sequence of the antisense strand has no mismatches to an equal length portion of SEQ ID NO: 1. In some embodiments, the mismatch is at position 1 of the antisense strand, counting from the 5′ terminus of the antisense strand.

In some embodiments, the sense strand is 15 to 25 nucleotides in length. In some embodiments, the sense strand is 17 to 23 nucleotides in length. In some embodiments, the sense strand is 19 to 23 nucleotides in length. In some embodiments, the sense strand is 19 to 21 nucleotides in length. In some embodiments, the sense strand is 21 to 23 nucleotides in length. In some embodiments, the sense strand is 15 nucleotides in length. In some embodiments, the sense strand is 16 nucleotides in length. In some embodiments, the sense strand is 17 nucleotides in length. In some embodiments, the sense strand is 18 nucleotides in length. In some embodiments, the sense strand is 19 nucleotides in length. In some embodiments, the sense strand is 20 nucleotides in length. In some embodiments, the sense strand is 21 nucleotides in length. In some embodiments, the sense strand is 22 nucleotides in length. In some embodiments, the sense strand is 23 nucleotides in length. In some embodiments, the sense strand is 24 nucleotides in length. In some embodiments, the sense strand is 25 nucleotides in length.

In some embodiments, the length of the sense strand is identical to the length of the antisense strand. In some embodiments, the length of the sense strand is greater than the length of the antisense strand. In some embodiments, the length of the sense strand is less than the length of the antisense strand.

The double stranded or duplex region of the double stranded nucleic acid may be from 15 to 25 nucleobase pairs in length, depending on the lengths of the sense strand and the antisense strand. In some embodiments, the double stranded region is 17 to 23 nucleobase pairs in length. In some embodiments, the double stranded region is 19 to 21 nucleobase pairs in length. In some embodiments, the double stranded region is 21 to 23 nucleotides in length. In some embodiments, the double stranded region is 15 nucleobase pairs in length. In some embodiments, the double stranded region is 16 nucleobase pairs in length. In some embodiments, the double stranded region is 17 nucleobase pairs in length. In some embodiments, the double stranded region is 18 nucleobase pairs in length. In some embodiments, the double stranded region is 19 nucleobase pairs in length. In some embodiments, the double stranded region is 20 nucleobase pairs in length. In some embodiments, the double stranded region is 21 nucleobase pairs in length. In some embodiments, the double stranded region is 22 nucleobase pairs in length. In some embodiments, the double stranded region is 23 nucleobase pairs in length. In some embodiments, the double stranded region is 24 nucleobase pairs in length. In some embodiments, the double stranded region is 25 nucleobase pairs in length.

In some embodiments, the nucleotide sequence of the sense strand has no more than one mismatch to the nucleotide sequence of the antisense strand of the double stranded nucleic acid. In some embodiments, the nucleotide sequence of the sense strand has no more than two mismatches to the nucleotide sequence of the antisense strand of the double stranded nucleic acid. In some embodiments, the nucleotide sequence of the sense strand has no mismatches to the nucleotide sequence of an antisense strand of the double stranded nucleic acid. In some embodiments, the nucleotide sequence of the sense strand has one mismatch to the nucleotide sequence of the antisense strand of the double stranded nucleic acid. In some embodiments, the mismatch is at position 1 of the sense strand, counting from the 3′ terminus of the sense strand. Single stranded nucleotide overhangs and nucleotide linkers are not considered for the purposes of determining the number of mismatches within the double stranded region of a double stranded nucleic acid provided herein. For example, a double stranded nucleic acid comprising an antisense strand that is 23 nucleotides in length, and a sense strand that is 21 nucleotides in length have no mismatches over the double stranded region, provided the nucleotide sequence of the sense strand is fully complementary over its length to the nucleotide sequence of the antisense strand. Alternatively, a double stranded nucleic acid comprising a sense strand that is 20 nucleotides in length, an antisense strand that is 22 nucleotides in length, and a nucleotide linker that is eight nucleotides in length, may have no mismatches over the double stranded region provided the nucleotide sequence of the sense strand is fully complementary over its length to the nucleotide sequence of the antisense strand.

In some embodiments, the double stranded nucleic acid comprises an antisense strand of 19 nucleotides in length and a sense strand of 19 nucleotides in length. In some embodiments, the antisense strand is 22 nucleotides in length and the sense strand is 20 nucleotides in length. In some embodiments, the antisense strand is 23 nucleotides in length and the sense strand is 21 nucleotides in length. In some embodiments, the antisense strand is 23 nucleotides in length including two deoxythymidines at the 3′ terminus, and the sense strand is 21 nucleotides in length including two deoxythymidines at the 3′ terminus. In some embodiments, the double stranded nucleic acid comprises an antisense strand of 21 nucleotides in length and a sense strand of 19 nucleotides in length.

In some embodiments of the double stranded nucleic acid where the antisense strand and sense strand are separate strands that are not covalently linked, the terminal nucleotides may form a nucleobase pair, in which case the end of the double stranded nucleic acid is a blunt end. Alternatively, one or more unpaired nucleotides of an antisense strand and/or sense strand may extend beyond the terminus of the complementary strand, resulting in a nucleotide overhang of one or more terminal single stranded nucleotides. In some embodiments, at least one of the 5′ and 3′ terminus of the double stranded nucleic acid is a blunt end. In some embodiments, both the 5′ terminus and 3′ terminus of the double stranded nucleic acid are blunt ends. In some embodiments, at least one end of the double stranded nucleic acid comprises a nucleotide overhang. In some embodiments, each end of the double stranded nucleic acid comprises a nucleotide overhang. In some embodiments, one end of the double stranded nucleic acid is a blunt end and the other end of the double stranded nucleic acid comprises a nucleotide overhang. In some embodiments, the antisense strand comprises a nucleotide overhang at its 3′ terminus. In some embodiments, the sense strand comprises a nucleotide overhang at its 3′ terminus. In some embodiments, each of the antisense strand and sense strand comprises a nucleotide overhang at its 3′ terminus. In some embodiments, at least one of the antisense strand and sense strand comprises a nucleotide overhang at its 5′ terminus. In some embodiments, each of the antisense strand and sense strand comprises a nucleotide overhang at each 5′ terminus.

In some embodiments, a nucleotide overhang is from one to five single stranded nucleotides. In some embodiments, a nucleotide overhang is one single stranded nucleotide. In some embodiments, a nucleotide overhang is two single stranded nucleotides. In some embodiments, a nucleotide overhang is three single stranded nucleotides. In some embodiments, a nucleotide overhang is three single stranded nucleotides. In some embodiments, a nucleotide overhang is four single stranded nucleotides. In some embodiments, a nucleotide overhang is five single stranded nucleotides. In some embodiments, the antisense strand comprises a 3′nucleotide overhang, wherein the 3′ nucleotide overhang is two single stranded two nucleotides in length. In some embodiments, at least one of the single stranded nucleotides of a nucleotide overhang is a modified nucleotide. In some embodiments, each of the single stranded nucleotides of a nucleotide overhang is a modified nucleotide. In some embodiments, the modified nucleotide is a 2′-O-methyl nucleotide. In some embodiments, the nucleotide overhang is two single stranded nucleotides and each nucleotide is a 2′-O-methyl nucleotide.

In some embodiments, at least one nucleotide of the nucleotide overhang at the 3′ terminus of an antisense strand is complementary to a corresponding nucleotide of SEQ ID NO: 1. In some embodiments, each nucleotide of the nucleotide overhang at the 3′ terminus of an antisense strand is complementary to a corresponding nucleotide of SEQ ID NO: 1. In some embodiments, at least one nucleotide of the nucleotide overhang at the 3′ terminus of an antisense strand is not complementary to a corresponding nucleotide of SEQ ID NO: 1. In some embodiments, each nucleotide of the nucleotide overhang at the 3′ terminus of an antisense strand is not complementary to a corresponding nucleotide of SEQ ID NO: 1.

In some embodiments, the antisense strand is 21 nucleotides in length, the sense strand is 19 nucleotides in length, the duplex is 19 nucleotide pairs in length, and the nucleotide sequence of the antisense strand is at least about 95%, at least about 96%, at least about 97%, at least about 98%, or at least about 99% complementary to the nucleotide sequence of the sense strand in the double stranded or duplex region.

In some embodiments, the antisense strand is 21 nucleotides in length, the sense strand is 19 nucleotides in length, the duplex is 19 nucleotide pairs in length, and the nucleotide sequence of the antisense strand is about 95% complementary to the nucleotide sequence of the sense strand in the double stranded or duplex region.

In some embodiments, the antisense strand is 21 nucleotides in length and the sense strand is 19 nucleotides in length, the nucleotide sequence of the antisense strand is fully complementary to the nucleotide sequence of the sense strand over the double stranded region, the length of the double stranded region is 19 nucleobase pairs.

In some embodiments, the antisense strand is 19 nucleotides in length and the sense strand is 19 nucleotides in length, the nucleotide sequence of the antisense strand is fully complementary to the nucleotide sequence of the sense strand over the double stranded region, and the length of the double stranded region is 19 nucleobase pairs and each terminus is a blunt end.

In some embodiments of the double stranded nucleic acid comprising a nucleotide linker, the termini that are not connected by the nucleotide linker may form a blunt end or may form a nucleotide overhang of one or more single stranded nucleotides. In some embodiments, the non-linked end of the double stranded nucleic acid is a blunt end. In some embodiments, the non-linked end comprises a nucleotide overhang of one or more single stranded nucleotides. In some embodiments, the non-linked end of the antisense strand comprises a nucleotide overhang. In some embodiments, the non-linked end of the sense strand comprises a nucleotide overhang. In some embodiments, the 3′ terminus of the antisense strand comprises a nucleotide overhang. In some embodiments, the 3′ terminus of the sense strand comprises a nucleotide overhang. In some embodiments, the 5′ terminus of the sense strand comprises a nucleotide overhang. In some embodiments, the 5′ terminus of the sense strand comprises a nucleotide overhang.

In some embodiments, the antisense strand comprises at least 17 contiguous nucleotides from the nucleotide sequence of any one of SEQ ID Nos: 294 to 584. In some embodiments, the antisense strand comprises at least 18 contiguous nucleotides from the nucleotide sequence of any one of SEQ ID Nos: 294 to 584. In some embodiments, the antisense strand comprises at least 19 contiguous nucleotides from the nucleotide sequence of any one of SEQ ID Nos: 294 to 584. In some embodiments, the antisense strand comprises at least 20 contiguous nucleotides from the nucleotide sequence of any one of SEQ ID Nos: 294 to 584. In some embodiments, the antisense strand comprises at least 21 contiguous nucleotides from the nucleotide sequence of any one of SEQ ID Nos: 294 to 584. In some embodiments, the antisense strand comprises at least 22 contiguous nucleotides from the nucleotide sequence of any one of SEQ ID Nos: 294 to 584. In some embodiments, the antisense strand comprises at least 23 contiguous nucleotides from the nucleotide sequence of any one of SEQ ID Nos: 294 to 584.

In some embodiments, the antisense strand comprises at least 15 contiguous nucleotides differing by no more than 3 nucleotides from any one of the antisense sequences listed in SEQ ID NO 294-584. In some embodiments, the antisense strand comprises at least 15 contiguous nucleotides differing by no more than 2 nucleotides from any one of the antisense sequences listed in SEQ ID NO 294-584. In some embodiments, the antisense strand comprises at least 15 contiguous nucleotides differing by no more than 0 or 1 nucleotide from any one of the antisense sequences listed in SEQ ID NO 294-584. In some embodiments, the antisense strand comprises at least 16, at least 17, at least 18, or at least 19 contiguous nucleotides differing by no more than 3 nucleotides from any one of the antisense sequences listed in SEQ ID NO 294-584. In some embodiments, the antisense strand comprises at least 16, at least 17, at least 18, or at least 19 contiguous nucleotides differing by no more than 2 nucleotides from any one of the antisense sequences listed in SEQ ID NO 294-584. In some embodiments, the antisense strand comprises at least 16, at least 17, at least 18, or at least 19 contiguous nucleotides differing by no more than 0 or 1 nucleotide from any one of the antisense sequences listed in SEQ ID NO 294-584.

In some embodiments, the sense strand comprises at least 17 contiguous nucleotides from the nucleotide sequence of any one of SEQ ID Nos: 3 to 293. In some embodiments, the sense strand comprises at least 18 contiguous nucleotides from the nucleotide sequence of any one of SEQ ID Nos: 3 to 293. In some embodiments, the sense strand comprises at least 19 contiguous nucleotides from the nucleotide sequence of any one of SEQ ID Nos: 3 to 293. In some embodiments, the sense strand comprises at least 20 contiguous nucleotides from the nucleotide sequence of any one of SEQ ID Nos: 3 to 293. In some embodiments, the sense strand comprises at least 21 contiguous nucleotides from the nucleotide sequence of any one of SEQ ID Nos: 3 to 293. In some embodiments, the sense strand comprises at least 22 contiguous nucleotides from the nucleotide sequence of any one of SEQ ID Nos: 3 to 293. In some embodiments, the sense strand comprises at least 23 contiguous nucleotides from the nucleotide sequence of any one of SEQ ID Nos: 3 to 293.

In some embodiments, the sense strand comprises at least 15 contiguous nucleotides differing by no more than 3 nucleotides from any one of the sense sequences listed in SEQ ID NO 3-293. In some embodiments, the sense strand comprises at least 15 contiguous nucleotides differing by no more than 2 nucleotides from any one of the sense sequences listed in SEQ ID NO 3-293. In some embodiments, the sense strand comprises at least 15 contiguous nucleotides differing by no more than 0 or 1 nucleotide from any one of the sense sequences listed in SEQ ID NO 3-293. In some embodiments, the sense strand comprises at least 16, at least 17, at least 18, or at least 19 contiguous nucleotides differing by no more than 3 nucleotides from any one of the sense sequences listed in SEQ ID NO 3-293. In some embodiments, the sense strand comprises at least 16, at least 17, at least 18, or at least 19 contiguous nucleotides differing by no more than 2 nucleotides from any one of the antisense sequences listed in SEQ ID NO 3-293. In some embodiments, the sense strand comprises at least 16, at least 17, at least 18, or at least 19 contiguous nucleotides differing by no more than 0 or 1 nucleotide from any one of the antisense sequences listed in SEQ ID NO: 3-293.

In some embodiments, the antisense strand comprises at least 17 contiguous nucleotides from the nucleotide sequence of any one of SEQ ID Nos: 294 to 584, and the sense strand comprises at least 17 contiguous nucleotides from the nucleotide sequence of any one of SEQ ID Nos: 3 to 293. In some embodiments, the antisense strand comprises at least 18 contiguous nucleotides from the nucleotide sequence of any one of SEQ ID Nos: 294 to 584, and the sense strand comprises at least 18 contiguous nucleotides from the nucleotide sequence of any one of SEQ ID Nos: 3 to 293. In some embodiments, the antisense strand comprises at least 19 contiguous nucleotides from the nucleotide sequence of any one of SEQ ID Nos: 294 to 584, and the sense strand comprises at least 19 contiguous nucleotides from the nucleotide sequence of any one of SEQ ID Nos: 3 to 293. In some embodiments, the antisense strand comprises at least 20 contiguous nucleotides from the nucleotide sequence of any one of SEQ ID Nos: 294 to 584, and the sense strand comprises at least 19 contiguous nucleotides from the nucleotide sequence of any one of SEQ ID Nos: 3 to 293. In some embodiments, the antisense strand comprises at least 21 contiguous nucleotides from the nucleotide sequence of any one of SEQ ID Nos: 294 to 584, and the sense strand comprises at least 19 contiguous nucleotides from the nucleotide sequence of any one of SEQ ID Nos: 3 to 293.

In some embodiments, the antisense strand comprises at least 15 contiguous nucleotides differing by no more than 3 nucleotides from any one of the antisense sequences listed in SEQ ID NO 294-584, and the sense strand comprises at least 15 contiguous nucleotides differing by no more than 3 nucleotides from any one of the sense sequences listed in SEQ ID NO 3-293. In some embodiments, the antisense strand comprises at least 15 contiguous nucleotides differing by no more than 2 nucleotides from any one of the antisense sequences listed in SEQ ID NO 294-584, and the sense strand comprises at least 15 contiguous nucleotides differing by no more than 2 nucleotides from any one of the sense sequences listed in SEQ ID NO 3-293. In some embodiments, the antisense strand comprises at least 15 contiguous nucleotides differing by no more than 0 or 1 nucleotide from any one of the antisense sequences listed in SEQ ID NO 294-584, and the sense strand comprises at least 15 contiguous nucleotides differing by no more than 0 or 1 nucleotide from any one of the sense sequences listed in SEQ ID NO 3-293. In some embodiments, the antisense strand comprises at least 16, at least 17, at least 18, or at least 19 contiguous nucleotides differing by no more than 3 nucleotides from any one of the antisense sequences listed in SEQ ID NO 294-584, and the sense strand comprises at least 16, at least 17, at least 18, or at least 19 contiguous nucleotides differing by no more than 3 nucleotides from any one of the sense sequences listed in SEQ ID NO 3-293. In some embodiments, the antisense strand comprises at least 16, at least 17, at least 18, or at least 19 contiguous nucleotides differing by no more than 2 nucleotides from any one of the antisense sequences listed in SEQ ID NO 294-584, and the sense strand comprises at least 16, at least 17, at least 18, or at least 19 contiguous nucleotides differing by no more than 2 nucleotides from any one of the antisense sequences listed in SEQ ID NO 3-293. In some embodiments, the antisense strand comprises at least 16, at least 17, at least 18, or at least 19 contiguous nucleotides differing by no more than 0 or 1 nucleotide from any one of the antisense sequences listed in SEQ ID NO 294-584, and the sense strand comprises at least 16, at least 17, at least 18, or at least 19 contiguous nucleotides differing by no more than 0 or 1 nucleotide from any one of the antisense sequences listed in SEQ ID NO: 3-293.

In some embodiments, the nucleotide sequence of the antisense strand includes at least 15 contiguous nucleotides of a nucleotide sequence selected from any one of SEQ ID Nos: 294 to 584, and the nucleotide sequence of the sense strand has no more than two mismatches to the nucleotide sequence of the antisense strand. In some embodiments, the nucleotide sequence of the antisense strand includes at least 16 contiguous nucleotides of a nucleotide sequence selected from any one of SEQ ID Nos: 294 to 584, and the nucleotide sequence of the sense strand has no more than two mismatches to the nucleotide sequence of the antisense strand. In some embodiments, the nucleotide sequence of the antisense strand includes at least 17 contiguous nucleotides of a nucleotide sequence selected from any one of SEQ ID Nos: 294 to 584, and the nucleotide sequence of the sense strand has no more than two mismatches to the nucleotide sequence of the antisense strand. In some embodiments, the nucleotide sequence of the antisense strand includes at least 18 contiguous nucleotides of a nucleotide sequence selected from any one of SEQ ID Nos: 294 to 584, and the nucleotide sequence of the sense strand has no more than two mismatches to the nucleotide sequence of the antisense strand. In some embodiments, the nucleotide sequence of the antisense strand includes at least 19 contiguous nucleotides of a nucleotide sequence selected from any one of SEQ ID Nos: 294 to 584, and the nucleotide sequence of the sense strand has no more than two mismatches to the nucleotide sequence of the antisense strand. In some embodiments, the nucleotide sequence of the antisense strand includes at least 20 contiguous nucleotides of a nucleotide sequence selected from any one of SEQ ID Nos: 294 to 584, and the nucleotide sequence of the sense strand has no more than two mismatches to the nucleotide sequence of the antisense strand. In some embodiments, the nucleotide sequence of the antisense strand includes at least 21 contiguous nucleotides of a nucleotide sequence selected from any one of SEQ ID Nos: 294 to 584, and the nucleotide sequence of the sense strand has no more than two mismatches to the nucleotide sequence of the antisense strand.

In some embodiments, the nucleotide sequence of the antisense strand comprises at least 21 contiguous nucleotides of a nucleotide sequence selected from any one of SEQ ID Nos: 294 to 584, and the nucleotide sequence of the sense strand has no more than 0 or 1 mismatch to the nucleotide sequence of the antisense strand.

In some embodiments, the nucleotide sequence of the antisense strand includes 21 contiguous nucleotides of a nucleotide sequence selected from any one of SEQ ID Nos: 294 to 584, and the nucleotide sequence of the sense strand has 0 or 1 mismatch to the nucleotide sequence of the sense strand.

In some embodiments, the antisense strand comprises any one of the sequences listed in SEQ ID NO: 294-584.

In some embodiments, the antisense strand is identical to a nucleotide sequence selected from any one of SEQ ID NO: 294 to 584.

In some embodiments, the antisense strand comprises or consists of a nucleic acid sequence that differs by 0 or 1 nucleotide selected from the group consisting of

(SEQ ID NO: 549)
UCAAUAAAUAUCCAAACCGCC;
and
(SEQ ID NO: 573)
UAGACAAUAAAUACCGAGGAA.

In some embodiments, the sense strand comprises any one of the sequences listed in SEQ ID NO: 3-293.

In some embodiments, the sense strand is identical to a nucleotide sequence selected from any one of SEQ ID NO: 3-293.

In some embodiments, the sense strand comprises or consists of a nucleic acid sequence that differs by 0 or 1 nucleotide selected from the group consisting of

(SEQ ID NO: 258)
CGGUUUGGAUAUUUAUUGA;
and
(SEQ ID NO: 282)
CCUCGGUAUUUAUUGUCUA.

In some embodiments, the double stranded nucleic acid comprises an antisense strand selected from SEQ ID NO: 294 to 584, and a sense strand selected from SEQ ID Nos: 3 to 293.

In some embodiments, the double stranded nucleic acid comprises an antisense strand selected from SEQ ID NO: 294 to 584, and a sense strand selected from SEQ ID Nos: 3 to 293, wherein the sense strand has no more than 0 or 1 mismatch to the nucleotide sequence of the antisense strand.

In some embodiments, the double stranded nucleic acid comprises an antisense strand comprising or consisting of a nucleic acid sequence that differs by 0 or 1 nucleotide selected from the group consisting of

(SEQ ID NO: 549)
UCAAUAAAUAUCCAAACCGCC;
and
(SEQ ID NO: 573)
UAGACAAUAAAUACCGAGGAA,

and a sense strand comprising or consisting of a nucleic acid sequence that differs by 0 or 1 nucleotide selected from the group consisting of

(SEQ ID NO: 258)
CGGUUUGGAUAUUUAUUGA;
and
(SEQ ID NO: 282)
CCUCGGUAUUUAUUGUCUA.

In some embodiments, the double stranded nucleic acid comprises an antisense strand comprising or consisting of a nucleic acid sequence that differs by 0 or 1 nucleotide selected from the group consisting of

(SEQ ID NO: 549)
UCAAUAAAUAUCCAAACCGCC

and a sense strand comprising or consisting of a nucleic acid sequence that differs by 0 or 1 nucleotide selected from the group consisting of

(SEQ ID NO: 258)
CGGUUUGGAUAUUUAUUGA.

In some embodiments, the double stranded nucleic acid comprises an antisense strand comprising or consisting of a nucleic acid sequence that differs by 0 or 1 nucleotide selected from the group consisting of

(SEQ ID NO: 573)
UAGACAAUAAAUACCGAGGAA,

and a sense strand comprising or consisting of a nucleic acid sequence that differs by 0 or 1 nucleotide selected from the group consisting of

(SEQ ID NO: 282)
CCUCGGUAUUUAUUGUCUA.

In some embodiments, at least one nucleotide of the sense strand and antisense strand is a modified nucleotide and/or includes a modified internucleoside linkage. In some embodiments, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 nucleotides of the sense strand and/or the antisense strand are modified nucleotides.

In some embodiments, all of the nucleotides of the sense strand and the antisense strand are modified nucleotides (vide infra).

### Modified Nucleotides

Double stranded nucleic acids provided herein may comprise one or more modified nucleotides. A modified nucleotide may be selected over an unmodified form because of desirable properties such as, for example, enhanced cellular uptake, enhanced affinity for other oligonucleotides or nucleic acid targets, increased stability in the presence of nucleases, and/or reduced immune stimulation.

In some embodiments, at least one nucleotide of the antisense strand is a modified nucleotide. In some embodiments, at least one nucleotide of the sense strand is a modified nucleotide. In some embodiments, each nucleotide of the antisense strand forming the double stranded region is a modified nucleotide. In some embodiments, each nucleotide of the sense strand forming the double stranded region is a modified nucleotide.

In some embodiments, a modified nucleotide comprises one or more of a modified sugar moiety, a modified internucleoside linkage, a modified nucleobase, and a 5′-terminal modified phosphate group. In some embodiments, a modified nucleotide comprises a modified sugar moiety. In some embodiments, a modified nucleotide comprises a modified internucleoside linkage. In some embodiments, a modified nucleotide comprises a modified nucleobase. In some embodiments, a modified nucleotide comprises a modified 5′-terminal phosphate group. In some embodiments, a modified nucleotide comprises a modification at the 5′ carbon of the pentafuranosyl sugar. In some embodiments, a modified nucleotide comprises a modification at the 3′ carbon of the pentafuranosyl sugar. In some embodiments, a modified nucleotide comprises a modification at the 2′ carbon of the pentafuranosyl sugar. In some embodiments, a modified nucleotide is at the 5′ terminus of an antisense strand or sense strand. In some embodiments, a modified nucleotide is at the 3′ terminus of an antisense strand or sense strand. In some embodiments, a modified nucleotide is at an internal nucleotide of an antisense strand or sense strand. In some embodiments, a modified nucleotide comprises a ligand attached to the 2′, 3′, or 5′ carbon of the pentafuranosyl sugar. In some embodiments, a nucleotide comprises a ligand attached to a nucleobase.

A modified nucleotide may comprise a modified sugar moiety, a naturally occurring nucleobase, and a naturally occurring internucleoside linkage. A modified nucleotide may comprise a modified sugar moiety, a naturally occurring nucleobase, and a modified internucleoside linkage.

In some embodiments, a modified sugar moiety is modified at the 2′ carbon of the pentafuranosyl sugar, relative to the naturally occurring 2′-OH of RNA or the 2′-H of DNA. In some embodiments, a modification at the 2′ carbon of the pentafuranosyl sugar is selected from —F, —OCF₃, —OCH₃ (also referred to as “2′-OMe” or “2′-O-methyl”), —OCH₂CH₂OCH₃ (also referred to as “2′-O-methoxyethyl” or “2′-MOE”), 2′-O(CH₂)₂SCH₃, —O—(CH₂)₂—O—N(CH₃)₂, —O(CH₂)₂O(CH₂)₂N(CH₃)₂, and —O—CH₂—C(═O)—N(H)CH₃.

In some embodiments, a modified sugar moiety is a 2′-fluoro sugar (also referred to as a 2′-F sugar). In some embodiments, a modified sugar moiety is a 2′-O-methyl sugar (also referred to as a “2′-OMe sugar” or a “2′-OCH₃” sugar). In some embodiments, a modified sugar moiety is a 2′-O-methoxyethyl sugar (also referred to as a 2′-OCH₂CH₂OCH₃ or a 2′-MOE sugar).

In some embodiments, the modified nucleotide comprising a modified sugar moiety is selected from a 2′-fluoro nucleotide, a 2′-O-methyl nucleotide, a 2′-O-methoxyethyl nucleotide, and a bicyclic sugar nucleotide. In some embodiments, a modified nucleotide is a 2′-fluoro nucleotide, where the 2′ carbon of the pentafuranosyl sugar has a fluoro substitution. In some embodiments, a modified nucleotide is a 2′-O-methyl nucleotide, where the 2′ carbon of the pentafuranosyl sugar has a 2′-O methyl substitution. In some embodiments, a modified nucleotide is a 2′-O-methoxyethyl nucleotide, where the 2′ carbon of the pentafuranosyl sugar has a 2′-O-methoxyethyl substitution. Other modified nucleotides may be similarly named.

In some embodiments, a modified nucleotide comprises a modified sugar moiety, where the ribose has a covalent linkage between the 2′ and 4′ carbons. Such a modified sugar moiety may be referred to as a “bicyclic sugar”, and nucleotides comprising such sugar moieties may be referred to as “bicyclic nucleic acids”. In some embodiments, the covalent linkage of a bicyclic sugar is a methyleneoxy linkage (4′-CH₂—O-2′), also known as “LNA”. In some embodiments, the covalent linkage of a bicyclic sugar is an ethyleneoxy linkage (4′-(CH₂)₂—O-2′), also known as “ENA”. In some embodiments, the covalent linkage of a bicyclic moiety is a methyl(methyleneoxy) linkage (4′-CH(CH₃)—O-2′), also known as “constrained ethyl” or “cEt.” In certain some embodiments, the —CH(CH₃)— bridge is constrained in the S orientation (“S-cEt”). In certain some embodiments, the —CH(CH₃)— bridge is constrained in the R orientation (“R-cEt”). In some embodiments, the covalent linkage of a bicyclic sugar is a (4′-CH(CH₂—OMe)-O-2′ linkage, also known as “c-MOE”. In some embodiments, the bicyclic sugar is a D sugar in the alpha configuration. In certain such embodiments, the bicyclic sugar is a D sugar in the beta configuration. In certain such embodiments, the bicyclic sugar is an L sugar in the alpha configuration. In certain such embodiments, the bicyclic sugar is an L sugar in the beta configuration.

In some embodiments, a modified sugar moiety is a 1,5-anhydrohexitol nucleic acid, also known as a “hexitol nucleic acid” or “HNA.”

In some embodiments, the oxygen of the pentafuranosyl sugar is replaced with a sulfur, to form a thio-sugar. In some embodiments, a thio-sugar is modified at the 2′ carbon.

In some embodiments, a modified internucleoside linkage is a phosphorothioate internucleoside linkage. In some embodiments, a modified internucleoside linkage is a methylphosphonate internucleoside linkage.

In some embodiments, the first two internucleoside linkages at the 5′ terminus of the sense strand and the last two internucleoside linkages at the 3′ terminus of the sense strand are phosphorothioate internucleoside linkages. In some embodiments, the first two internucleoside linkages at the 5′ terminus of the antisense strand and the last two internucleoside linkages at the 3′ terminus of the antisense strand are phosphorothioate internucleoside linkages. In some embodiments, the first two internucleoside linkages at the 5′ terminus of the sense strand and the last two internucleoside linkages at the 3′ terminus of the sense strand are phosphorothioate internucleoside linkages, and the first two internucleoside linkages at the 5′ terminus of the antisense strand and the last two internucleoside linkages at the 3′ terminus of the antisense strand are phosphorothioate internucleoside linkages.

In some embodiments, a modified nucleobase is selected from 5-hydroxymethyl cytosine, 7-deazaguanine and 7-deazaadenine. In some embodiments, a modified nucleobase is selected from 7-deaza-adenine, 7-deazaguanosine, 2-aminopyridine and 2-pyridone. In some embodiments, a modified nucleobase is selected from 5-substituted pyrimidines, 6-azapyrimidines and N-2, N-6 and 0-6 substituted purines, including 2 aminopropyladenine, 5-propynyluracil and 5-propynylcytosine.

In some embodiments, a modified nucleotide comprises a modification of the phosphate group at the 5′-carbon of the pentafuranosyl sugar. In some embodiments, the modified phosphate group is 5′-(E)-vinylphosphonate (5′-VP).

In some embodiments, a modified nucleotide is a phosphorodiamidite-linked morpholino nucleotide.

In some embodiments, a modified nucleotide comprises an acyclic nucleoside derivative lacking the bond between the 2′ carbon and 3′ carbon of the sugar ring, also known as an “unlocked nucleic acid” or “UNA”. In some embodiments, the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the following Pattern I:
5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}N_{F}N_{M}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the following Pattern II:
5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}N_{M}N_{F}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleoside linkage, and each other internucleoside linkage is a phosphodiester internucleoside linkage.

In some embodiments, the antisense strand is 19 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides and nucleotides 2, 4, 6, 8, 10, 12, 14, 16, and 18 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the following Pattern III:
5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}^{S}N_{F}^{S}N_{M}-3′, wherein “NM” is a 2′-O-methyl nucleotide, “N_{F}” is a 2′-fluoro nucleotide, a superscript “S” is a phosphorothioate internucleoside linkage, and each other internucleoside linkages is a phosphodiester internucleoside linkage.

In some embodiments, the sense strand is 19 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleoside linkages at the 5′ terminus and the last two internucleoside linkages at the 3′ terminus are phosphorothioate internucleoside linkages, and each other internucleotide is a phosphodiester internucleoside linkage. Such a modification pattern may be represented by the following Pattern IV:
5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-flouro nucleotide, a superscript “S” is a phosphorothioate internucleoside linkage, and each other internucleoside linkage is a phosphorodiester internucleoside linkage.

In some embodiments, the sense strand is 19 nucleotides in length and the nucleotides of the sense strand are modified such that, counting from the 5′ terminus of the sense strand, nucleotides 5, 7, 9, 11, 13, and 15 are 2′-fluoro nucleotides and nucleotides 1, 2, 3, 4, 6, 8, 10, 12, 14, 16, 17, 18, and 19 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the following Pattern IV-1:
5′-N_{M}^{S}N_{M}^{S}N_{M}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “N_{M}” is a 2′-O-methyl nucleotide, “N_{F}” is a 2′-flouro nucleotide, a superscript “S” is a phosphorothioate internucleoside linkage, and each other internucleoside linkage is a phosphorodiester internucleoside linkage.

In some embodiments, the sense strand is 19 nucleotides in length and the nucleotides of the sense strand are modified such that, counting from the 5′ terminus of the sense strand, nucleotides 5, 8, 9, 11, and 13 are 2′-fluoro nucleotides and nucleotides 1, 2, 3, 4, 6, 7, 10, 12, 14, 15, 16, 17, 18, and 19 are 2′-O-methyl nucleotides, the first two internucleoside linkages at the 5′ terminus and the last two internucleoside linkages at the 3′ terminus are phosphorothioate internucleoside linkages, and each other internucleotide is a phosphodiester internucleoside linkage. Such a modification pattern may be represented by the following Pattern IV-2:
5′-N_{M}^{S}N_{M}^{S}N_{M}N_{M}N_{F}N_{M}N_{M}N_{F}N_{F}N_{M}N_{F}N_{M}N_{F}N_{M}N_{M}N_{M}N_{M}^{S}N_{M}^{S}N_{M}-3′, wherein “N_{M}” is a 2′-O-methyl nucleotide, “N_{F}” is a 2′-flouro nucleotide, a superscript “S” is a phosphorothioate internucleoside linkage, and each other internucleoside linkage is a phosphorodiester internucleoside linkage.

In some embodiments, the sense strand is 19 nucleotides in length and the nucleotides of the sense strand are modified such that, counting from the 5′ terminus of the sense strand, nucleotides 5, 7, 8, and 9 are 2′-fluoro nucleotides and nucleotides 1, 2, 3, 4, 6, 10, 11, 12, 13, 14, 15, 16, 17, 18, and 19 are 2′-O-methyl nucleotides, the first two internucleoside linkages at the 5′ terminus and the last two internucleoside linkages at the 3′ terminus are phosphorothioate internucleoside linkages, and each other internucleoside is a phosphodiester internucleoside linkage. Such a modification pattern may be represented by the following Pattern IV-3:
5′-N_{M}^{S}N_{M}^{S}N_{M}N_{M}N_{F}N_{M}N_{F}N_{F}N_{F}N_{M}N_{M}N_{M}N_{M}N_{M}N_{M}N_{M}N_{M}^{S}N_{M}^{S}N_{M}-3′, wherein “N_{M}” is a 2′-O-methyl nucleotide, “N_{F}” is a 2′-flouro nucleotide, a superscript “S” is a phosphorothioate internucleoside linkage, and each other internucleoside linkage is a phosphorodiester internucleoside linkage.

In some embodiments, the antisense strand is 23 nucleotides in length and the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the following Pattern V:
5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}N_{F}N_{M}N_{F}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, wherein the sense strand is 21 nucleotides in length and the nucleotides of the sense strand are modified such that, counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern VI:
5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the antisense strand is 23 nucleotides in length and the nucleotides of the antisense strand are modified such that, counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern VII:
5′-N_{M}^{S}N_{F}^{S}N_{M}N_{F}N_{M}N_{F}N_{M}N_{F}N_{M}N_{F}N_{M}N_{M}N_{M}N_{F}N_{M}N_{F}N_{M}N_{F}N_{M}N_{F}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 1, 3, 5, 7, 9, 10, 11, 13, 15, 17, 19, and 21 are 2′-fluoronucleotides, nucleotides 2, 4, 6, 8, 12, 14, 16, 18, and 20 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern VIII:
5′-N_{F}^{S}N_{M}^{S}N_{F}N_{M}N_{F}N_{M}N_{F}N_{M}N_{F}N_{F}N_{F}N_{M}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the antisense strand is 23 nucleotides in length and the nucleotides of the antisense strand are modified such that, counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern IX:
5′-N_{M}^{S}N_{F}^{S}N_{M}N_{F}N_{M}N_{F}N_{M}N_{F}N_{M}N_{M}N_{M}N_{F}N_{M}N_{F}N_{M}N_{F}N_{M}N_{F}N_{M}N_{F}N_{M}^{S}N_{M}^{S}N_{M}-3′, wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 1, 3, 5, 7, 9, 11, 12, 13, 15, 17, 19, and 21 are 2′-fluoro nucleotides, nucleotides 2, 4, 6, 8, 10, 14, 16, 18, and 20 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern X:
5′-N_{F}^{S}N_{M}^{S}N_{F}N_{M}N_{F}N_{M}N_{F}N_{M}N_{F}N_{M}N_{F}N_{F}N_{F}N_{M}N_{F}N_{M}N_{F}N_{M}N_{F}^{S}N_{M}^{S}N_{F}-3′, wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 23 nucleotides in length and the nucleotides of the sense strand are modified such that, counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, nucleotides 22 and 23 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XI:
5′-N_{F}^{S}N_{M}^{S}N_{F}N_{M}N_{F}N_{M}N_{F}N_{M}N_{F}N_{M}N_{F}N_{M}N_{F}N_{M}N_{F}N_{M}N_{F}N_{M}N_{F}N_{M}N_{F}^{S}N^{S}N_{F}-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleoside linkage, and each other internucleoside linkage is a phosphodiester internucleoside linkage.

In some embodiments, the sense strand is 23 nucleotides in length and the nucleotides of the sense strand are modified such that, counting from the 5′ terminus of the sense strand, nucleotides 1, 3, 5, 7, 9, 10, 11, 13, 15, 17, 19, and 21 are 2′-fluoro nucleotides, nucleotides 2, 4, 6, 8, 12, 14, 16, 18, and 20 are 2′-O-methyl nucleotides, nucleotides 22 and 23 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XII:

5′-N_{F}^{S}N_{M}^{S}N_{F}N_{M}N_{F}N_{M}N_{F}N_{M}NF_{N}F_{N}F_{N}M_{N}F_{N}M_{N}F_{N}M_{N}F_{N}M_{N}F_{N}M_{N}F^{S}N^{S}N-3′,

wherein “N_{M}” is a 2′-O-methyl nucleotide, “N_{F}” is a 2′-fluoro nucleotide, “N” is a beta-D-deoxynucleotide, a superscript “S” is a phosphorothioate internucleoside linkage, and each other internucleoside linkage is a phosphodiester internucleoside linkage.

In some embodiments, the sense strand is 23 nucleotides in length and the nucleotides of the sense strand are modified such that, counting from the 5′ terminus of the sense strand, nucleotides 1, 3, 5, 7, 9, 11, 12, 13, 15, 17, 19, and 21 are 2′-fluoronucleotides, nucleotides 2, 4, 6, 8, 10, 14, 16, 18, and 20 are 2′-O-methyl nucleotides, nucleotides 22 and 23 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XIII:
5′-N_{F}^{S}N_{M}^{S}N_{F}N_{M}N_{F}N_{M}N_{F}N_{M}N_{F}N_{M}N_{F}N_{F}N_{F}N_{M}N_{F}N_{M}N_{F}N_{M}N_{F}N_{M}N_{F}^{S}N^{S}N-3′, wherein “N_{M}” is a 2′-O-methyl nucleotide, “N_{F}” is a 2′-fluoro nucleotide, “N” is a beta-D-deoxynucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 1, 2, 3, 4, 6, 8, 12, 14, 16, 18, 19, 20, and 21 are 2′-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XIV:

5′-N_{M}^{S}N_{M}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 1, 2, 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XV:

5'-N_{M}^{S}N_{M}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the antisense strand is 23 nucleotides in length and the nucleotides of the antisense strand are modified such that, counting from the 5′ terminus of the antisense strand, nucleotides 1, 3, 5, 7, 8, 9, 11, 12, 13, 15, 17, 19, 21, 22, and 23 are 2′-O-methyl nucleotides, nucleotides 2, 4, 6, 10, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XVI:

5′-N_{M}^{S}N_{F}^{S}N_{M}N_{F}N_{M}N_{F}N_{M}N_{F}N_{M}N_{M}N_{M}N_{F}N_{M}N_{F}N_{M}N_{F}N_{M}N_{F}N_{M}N_{F}N_{M}^{S}N_{M}^{S}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the antisense strand is 23 nucleotides in length and the nucleotides of the antisense strand are modified such that, counting from the 5′ terminus of the antisense strand, nucleotides 1, 3, 4, 5, 7, 8, 9, 10, 11, 12, 13, 15, 17, 18, 19, 20, 21, 22, and 23 are 2′-O-methyl nucleotides, nucleotides 2, 6, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XVII:

5′-N_{M}^{S}N_{F}^{S}N_{M}N_{F}N_{M}N_{F}N_{M}N_{F}N_{M}N_{M}N_{M}N_{F}N_{M}N_{F}N_{M}N_{F}N_{M}N_{F}N_{M}N_{F}N_{M}^{S}N_{M}^{S}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XVIII:

5′-N_{M}^{S}N_{M}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleoside linkages at the 5′ terminus are phosphorothioate internucleoside linkages, and each other internucleoside linkage is a phosphodiester internucleoside linkage. Such a modification pattern may be represented by the Pattern XIX:

5′-N_{M}^{S}N_{M}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}N_{M}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 1 and 2 are 2′-O-methoxyethyl nucleotides, nucleotides 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XX:

5′-N_{E}^{S}N_{E}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′,

wherein “N_{E}” is a 2′-O-methoxyethyl nucleotide, “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 2 and 3 are 2′-O-methoxyethyl nucleotides, nucleotides 1, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XXI:

5′-N_{E}^{S}N_{E}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′,

wherein “N_{E}” is a 2′-O-methoxyethyl nucleotide, “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 2, 3, 19 and 20 are 2′-O-methoxyethyl nucleotides, nucleotides 1, 4, 6, 8, 12, 14, 16, 18, and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XXII:
5′-N_{E}^{S}N_{E}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′, wherein “N_{E}” is a 2′-O-methoxyethyl nucleotide, “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the sense strand is 21 nucleotides in length and the nucleotides of the sense strand are modified such that, counting from the 5′ terminus of the sense strand, nucleotides 1, 2, 3, and 4 are 2′-O-methoxyethyl nucleotides, nucleotides 6, 8, 12, 14, 16, 18, 19, 20 and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XXIII:

5′-N_{E}^{S}N_{E}^{S}N_{M}N_{M}N_{F}N_{M}N_{F}N_{M}N_{F}N_{M}N_{F}N_{M}N_{F}N_{M}N_{F}N_{M}N_{F}N_{M}N_{M}^{S}N_{M}^{S}N_{M}-3′,

wherein “N_{E}” is a 2′-O-methoxyethyl nucleotide, “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the antisense strand is 21 nucleotides in length and the nucleotides of the antisense strand are modified such that, counting from the 5′ terminus of the antisense strand, nucleotides 1, 3, 5, 7, 9, 11, 13, 15, 17, 19, 20, and 21 are 2′-O-methyl nucleotides, nucleotides 2, 4, 6, 8, 10, 12, 14, 16, 18 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XXIV:

5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}N_{F}N_{M}^{S}N_{M}^{S}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the Compound of Formula (I) is a dsRNAi agent, or a pharmaceutically acceptable salt thereof, comprising:
an antisense strand having a nucleotide sequence of 5′-(E)-vinylphosphonate-U_{M}^{S}C_{F}^{S}A_{M}A_{F}U_{M}A_{F}A_{M}A_{F}U_{M}A_{F}U_{M}C_{F}C_{M}A_{F}A_{M}A_{F}C_{M}C_{F}G_{M}^{S}C_{M}^{S}C_{M}-3′ (SEQ ID NO: 1312); and a sense strand having a nucleotide sequence of 5′-C_{F}^{S}G_{M}^{S}G_{F}U_{M}U_{F}U_{M}G_{F}G_{M}A_{F}U_{M}A_{F}U_{M}U_{F}U_{M}A_{F}U_{M}U_{F}^{S}G_{M}^{S}A_{F}-DTx-01-32-3′ (SEQ ID NO: 1311);
wherein, reading from the 5′ end to the 3′ end, the nucleotides followed by a subscript M have a 2′-O-methyl modification; the nucleotides followed by a subscript F have a 2′-fluoro modification; superscript S is a phosphorothioate internucleoside linkage; each other internucleoside linkage is a phosphodiester internucleoside linkage; and DTx-01-32 is

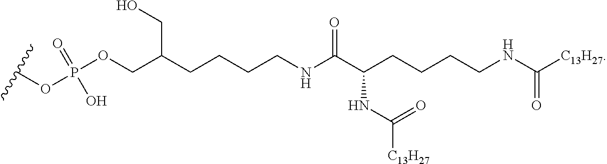

In some embodiments, the Compound of Formula (I) is a dsRNAi agent, or a pharmaceutically acceptable salt thereof, comprising:
an antisense strand having a nucleotide sequence of 5′-(E)-vinylphosphonate-U_{M}^{S}A_{F}^{S}G_{M}A_{F}C_{M}A_{F}A_{M}U_{F}A_{M}A_{F}A_{M}U_{F}A_{M}C_{F}C_{M}G_{F}A_{M}G_{F}G_{M}^{S}A_{M}^{S}A_{M}-3′ (SEQ ID NO: 1268); and a sense strand having a nucleotide sequence of 5′-C_{F}^{S}C_{M}^{S}U_{F}C_{M}G_{F}G_{M}U_{F}A_{M}U_{F}U_{M}U_{F}A_{M}U_{F}U_{M}G_{F}U_{M}C_{F}^{S}U_{M}^{S}A_{F}-DTx-01-32-3′ (SEQ ID NO: 1267);
wherein, reading from the 5′ end to the 3′ end, the nucleotides followed by a subscript M have a 2′-O-methyl modification; the nucleotides followed by a subscript F have a 2′-fluoro modification; superscript S is a phosphorothioate internucleoside linkage; each other internucleoside linkage is a phosphodiester internucleoside linkage; and DTx-01-32 is

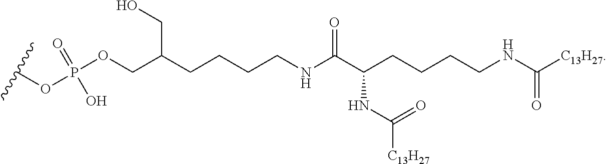

In some embodiments, the antisense strand is 21 nucleotides in length and the nucleotides of the antisense strand are modified such that, counting from the 5′ terminus of the antisense strand, nucleotides 1, 3, 5, 7, 9, 11, 13, 15, 17, 18, 19, 20, and 21 are 2′-O-methyl nucleotides, nucleotides 2, 4, 6, 8, 10, 12, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XXV:

5′-N_{M}^{S}N_{F}^{S}N_{M}N_{F}N_{M}N_{F}N_{M}N_{F}N_{M}N_{F}N_{M}N_{F}N_{M}N_{F}N_{M}N_{F}N_{M}N_{M}N_{M}^{S}N_{M}
^{S}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the Compound of Formula (I) is a dsRNAi agent, or a pharmaceutically acceptable salt thereof, comprising:
an antisense strand having a nucleotide sequence of 5′-(E)-vinylphosphonate-U_{M}^{S}C_{F}^{S}A_{M}A_{F}U_{M}A_{F}A_{M}A_{F}U_{M}A_{F}U_{M}C_{F}C_{M}A_{F}A_{M}A_{F}C_{M}C_{M}G_{M}^{S}C_{M}^{S}C_{M}-3′ (SEQ ID NO: 1346); and a sense strand having a nucleotide sequence of 5′-C_{M}^{S}G_{M}^{S}G_{M}U_{M}U_{F}U_{M}G_{F}G_{M}A_{F}U_{M}A_{F}U_{M}U_{F}U_{M}A_{F}U_{M}U_{M}^{S}G_{M}^{S}A_{M}-DTx-01-32-3′ (SEQ ID NO: 1345); wherein, reading from the 5′ end to the 3′ end, the nucleotides followed by a subscript M have a 2′-O-methyl modification; the nucleotides followed by a subscript F have a 2′-fluoro modification; superscript S is a phosphorothioate internucleoside linkage; each other internucleoside linkage is a phosphodiester internucleoside linkage; and DTx-01-32 is

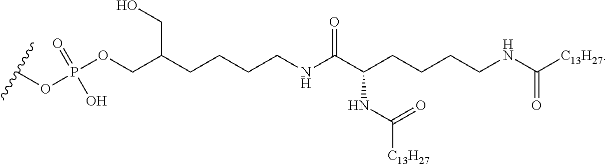

In some embodiments, the antisense strand is 21 nucleotides in length and the nucleotides of the antisense strand are modified such that, counting from the 5′ terminus of the antisense strand, nucleotides 1, 3, 4, 5, 7, 8, 9, 11, 13, 15, 17, 18, 19, 20, and 21 are 2′-O-methyl nucleotides, nucleotides 2, 6, 10, 12, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XXVI:

5′-N_{M}^{S}N_{F}^{S}N_{M}N_{M}N_{M}N_{F}N_{M}N_{M}N_{M}N_{F}N_{M}N_{F}N_{M}N_{F}N_{M}N_{F}N_{M}N_{M}N_{M}^{S}N_{M}
^{S}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the Compound of Formula (I) is a dsRNAi agent, or a pharmaceutically acceptable salt thereof, comprising:
an antisense strand having a nucleotide sequence of 5′-(E)-vinylphosphonate-U_{M}^{S}C_{F}^{S}A_{M}A_{M}U_{M}A_{F}A_{M}A_{M}U_{M}A_{F}U_{M}C_{F}C_{M}A_{F}A_{M}A_{F}C_{M}C_{M}G_{M}^{S}C_{M}^{S}C_{M}-3′ (SEQ ID NO: 1348); and a sense strand having a nucleotide sequence of 5′-C_{M}^{S}G_{M}^{S}G_{M}U_{M}U_{F}U_{M}G_{M}G_{F}A_{F}U_{M}A_{F}U_{M}U_{F}U_{M}A_{M}U_{M}U_{M}^{S}G_{M}^{S}A_{M}-DTx-01-32-3′ (SEQ ID NO: 1347); wherein, reading from the 5′ end to the 3′ end, the nucleotides followed by a subscript M have a 2′-O-methyl modification; the nucleotides followed by a subscript F have a 2′-fluoro modification; superscript S is a phosphorothioate internucleoside linkage; each other internucleoside linkage is a phosphodiester internucleoside linkage; and DTx-01-32 is

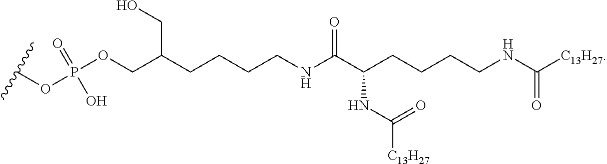

In some embodiments, the antisense strand is 21 nucleotides in length and the nucleotides of the antisense strand are modified such that, counting from the 5′ terminus of the antisense strand, nucleotides 1, 3, 4, 5, 7, 8, 9, 10, 11, 12, 13, 15, 17, 18, 19, 20, and 21 are 2′-O-methyl nucleotides, nucleotides 2, 6, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. Such a modification pattern may be represented by the Pattern XXVII:

5′-N_{M}^{S}N_{F}^{S}N_{M}N_{M}N_{M}N_{F}N_{M}N_{M}N_{M}N_{M}N_{M}N_{M}N_{M}N_{F}N_{M}N_{F}N_{M}N_{M}N_{M}^{S}N_{M}
^{S}N_{M}-3′,

wherein “N_{M}” is a 2′-O-methyl nucleotide, “N_{F}” is a 2′-fluoro nucleotide, a superscript “S” is a phosphorothioate internucleoside linkage, and each other internucleoside linkage is a phosphodiester internucleoside linkage.

In some embodiments, the Compound of Formula (I) is a dsRNAi agent, or a pharmaceutically acceptable salt thereof, comprising:
an antisense strand having a nucleotide sequence of 5′-(E)-vinylphosphonate-U_{M}^{S}C_{F}^{S}A_{M}A_{M}U_{M}A_{F}A_{M}A_{M}U_{M}A_{M}U_{M}C_{M}C_{M}A_{F}A_{M}A_{F}C_{M}C_{M}G_{M}^{S}C_{M}^{S}C_{M}-3′ (SEQ ID NO: 1352); and a sense strand having a nucleotide sequence of 5′-C_{M}^{S}G_{M}^{S}G_{M}U_{M}U_{F}U_{M}G_{F}G_{F}A_{F}U_{M}A_{M}U_{M}U_{M}U_{M}A_{M}U_{M}U_{M}^{S}G_{M}^{S}U_{M}-DTx-01-32-3′ (SEQ ID NO: 1351); wherein, reading from the 5′ end to the 3′ end, the nucleotides followed by a subscript M have a 2′-O-methyl modification; the nucleotides followed by a subscript F have a 2′-fluoro modification; superscript S is a phosphorothioate internucleoside linkage; each other internucleoside linkage is a phosphodiester internucleoside linkage; and DTx-01-32 is

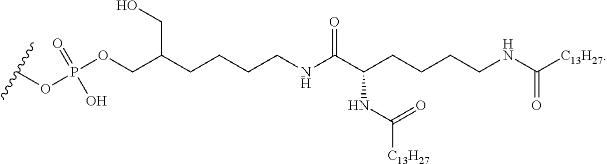

In some embodiments, the antisense strand has the modification pattern of Pattern I and a 5′-VP at the 5′-terminal nucleotide. In some embodiments, the antisense strand has the modification pattern of Pattern III and a 5′-VP at the 5′-terminal nucleotide. In some embodiments, the antisense strand has the modification pattern of Pattern V and a 5′-VP at the 5′-terminal nucleotide. In some embodiments, the antisense strand has the modification pattern of Pattern VII and a 5′-VP at the 5′ terminal nucleotide. In some embodiments, an antisense strand has the modification pattern of Pattern IX and a 5′-VP at the 5′ terminal nucleotide. In some embodiments, an antisense strand has the modification pattern of Pattern XVI and a 5′-VP at the 5′ terminal nucleotide. In some embodiments, an antisense strand has the modification pattern of Pattern XVII and a 5′-VP at the 5′ terminal nucleotide. In some embodiments, the antisense strand has the modification pattern of Pattern XXIV and a 5′-VP at the 5′ terminal nucleotide. In some embodiments, the antisense strand has the modification pattern of Pattern XXV and a 5′-VP at the 5′ terminal nucleotide. In some embodiments, an antisense strand has the modification pattern of Pattern XXVI and a 5′-VP at the 5′ terminal nucleotide. In some embodiments, an antisense strand has the modification pattern of Pattern XXVII and a 5′-VP at the 5′ terminal nucleotide.

In some embodiments, the compound includes an antisense strand and a sense strand hybridized to form a double stranded region, wherein the antisense strand and sense strand are not covalently linked, i.e., the antisense strand and sense strand form an siRNA, wherein the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern represented by Pattern I and the sense strand has the modification pattern represented by Pattern II.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 19 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides and nucleotides 2, 4, 6, 8, 10, 12, 14, 16, and 18 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotide in length and the nucleotides of the sense strand are modified such that, counting from the 5′ terminus of the sense strand, nucleotides 1, 3, 5, 7, 9, 11, 13, 15, 17, and 19 are 2′-fluoro nucleotides, nucleotides 2, 4, 6, 8, 10, 12, 14, 16, and 18 are 2′-O-methyl nucleotides, and nucleotides 20 and 21 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern represented by Pattern III and the sense strand has the modification pattern represented by Pattern II.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 21 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides, nucleotides 2, 4, 6, 8, 10, 12, 14, 16, and 18 are 2′-fluoro nucleotides, and nucleotides 20 and 21 are beta-D-deoxy nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 19 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern represented by Pattern I and the sense strand has the modification pattern represented by Pattern IV.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 19 nucleotides in length and the nucleotides of the antisense strand are modified such that, counting from the 5′ terminus of the antisense strand, nucleotides 1, 3, 5, 7, 9, 11, 13, 15, 17, and 19 are 2′-O-methyl nucleotides and nucleotides 2, 4, 6, 8, 10, 12, 14, 16, and 18 are 2′-fluoro nucleotides, the first two internucleoside linkages at the 5′ terminus and the last two internucleoside linkages at the 3′ terminus are phosphorothioate internucleoside linkages, and each other internucleotide is a phosphodiester internucleoside linkage; and wherein the sense strand is 19 nucleotides in length and the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern represented by Pattern III and the sense strand has the modification pattern represented by Pattern IV.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and the nucleotides of the sense strand are modified such that, counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern represented by Pattern V and the sense strand has the modification represented by Pattern VI.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 23 nucleotides in length and wherein the nucleotides of the antisense strand are modified such that, counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and the nucleotides of the sense strand are modified such that, counting from the 5′ terminus of the sense strand, nucleotides 1, 3, 5, 7, 9, 10, 11, 13, 15, 17, 19, and 21 are 2′-fluoronucleotides, nucleotides 2, 4, 6, 8, 12, 14, 16, 18, and 20 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern represented by Pattern VII and the sense strand has the modification pattern represented by Pattern VIII.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 23 nucleotides in length and wherein the nucleotides of the antisense strand are modified such that, counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and nucleotides of the sense strand are modified such that, counting from the 5′ terminus of the sense strand, nucleotides 1, 3, 5, 7, 9, 11, 12, 13, 15, 17, 19, and 21 are 2′-fluoro nucleotides, nucleotides 2, 4, 6, 8, 10, 14, 16, 18, and 20 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern IX and the sense strand has the modification pattern of Pattern X.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides and nucleotides 2, 4, 6, 8, 10, 12, 14, 16, 18, and 20 are 2′-fluoro nucleotides the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 23 nucleotides in length and wherein the nucleotides of the sense strand are modified such that, counting from the 5′ terminus of the sense strand, nucleotides 1, 3, 5, 7, 9, 11, 13, 15, 17, 19, and 21 are 2′-fluoro nucleotides, nucleotides 2, 4, 6, 8, 10, 12, 14, 16, 18, and 20 are 2′-O-methyl nucleotides, nucleotides 22 and 23 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern represented by Pattern V and the sense strand has the modification represented by Pattern XI.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 23 nucleotides in length and wherein the nucleotides of the antisense strand are modified such that, counting from the 5′ terminus of the antisense strand, nucleotides 1, 3, 5, 7, 9, 11, 12, 13, 15, 17, 19, 21, 22, and 23 are 2′-O-methyl nucleotides and nucleotides 2, 4, 6, 8, 10, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 23 nucleotides in length and wherein the nucleotides of the sense strand are modified such that, counting from the 5′ terminus of the sense strand, nucleotides 1, 3, 5, 7, 9, 10, 11, 13, 15, 17, 19, and 21 are 2′-fluoro nucleotides, nucleotides 2, 4, 6, 8, 12, 14, 16, 18, and 20 are 2′-O-methyl nucleotides, nucleotides 22 and 23 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern represented by Pattern VII and the sense strand has the modification pattern represented by Pattern XII.

In some embodiments, the compound includes an antisense strand and a sense strand hybridized to form a double stranded nucleic acid, wherein the antisense strand and the sense strand are not covalently linked, i.e., the antisense strand and sense strand form an siRNA, wherein the antisense strand is 23 nucleotides in length and wherein the nucleotides of the antisense strand are modified such, that counting from the 5′ terminus of the antisense strand, nucleotides 1, 3, 5, 7, 9, 10, 11, 13, 15, 17, 19, 21, 22, and 23 are 2′-O-methyl nucleotides and nucleotides 2, 4, 6, 8, 12, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 23 nucleotides in length and wherein the nucleotides of the sense strand are modified such that, counting from the 5′ terminus of the sense strand, nucleotides 1, 3, 5, 7, 9, 11, 12, 13, 15, 17, 19, and 21 are 2′-fluoro nucleotides, nucleotides 2, 4, 6, 8, 10, 14, 16, 18, and 20 are 2′-O-methyl nucleotides, nucleotides 22 and 23 are beta-D-deoxynucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern IX and the sense strand has the modification pattern of Pattern XIII.

In some embodiments, the compound includes an antisense strand and a sense strand hybridized to form a double stranded nucleic acid, wherein the antisense strand and the sense strand are not covalently linked, i.e., the antisense strand and sense strand form an siRNA, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and wherein the nucleotides of the sense strand are modified such that, counting from the 5′ terminus of the sense strand, nucleotides 1, 2, 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern V and the sense strand has the modification pattern of Pattern XIV.

In some embodiments, the compound includes an antisense strand and a sense strand hybridized to form a double stranded nucleic acid, wherein the antisense strand and the sense strand are not covalently linked, i.e., the antisense strand and sense strand form an siRNA, wherein the antisense strand is 23 nucleotides in length and the nucleotides of the antisense strand are modified such that, counting from the 5′ terminus of the antisense strand, nucleotides 1, 3, 5, 7, 8, 9, 11, 12, 13, 15, 17, 19, 21, 22, and 23 are 2′-O-methyl nucleotides, nucleotides 2, 4, 6, 10, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and the nucleotides of the sense strand are modified such that, counting from the 5′ terminus of the sense strand, nucleotides 1, 2, 3, 4, 5, 6, 8, 12, 14, 16, 18, 19, 20, and 21 are 2′-O-methyl nucleotides, nucleotides 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern XVI and the sense strand has the modification pattern of Pattern XV.

In some embodiments, the compound includes an antisense strand and a sense strand hybridized to form a double stranded nucleic acid, wherein the antisense strand and the sense strand are not covalently linked, i.e., the antisense strand and sense strand form an siRNA, wherein the antisense strand is 23 nucleotides in length and the nucleotides of the antisense strand are modified such that, counting from the 5′ terminus of the antisense strand, nucleotides 1, 3, 5, 7, 8, 9, 11, 12, 13, 15, 17, 19, 21, 22, and 23 are 2′-O-methyl nucleotides, nucleotides 2, 4, 6, 10, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern XVII and the sense strand has the modification pattern of Pattern XVIII.

In some embodiments, the compound includes an antisense strand and a sense strand hybridized to form a double stranded nucleic acid, wherein the antisense strand and the sense strand are not covalently linked, i.e., the antisense strand and sense strand form an siRNA, wherein the antisense strand is 23 nucleotides in length and the nucleotides of the antisense strand are modified such that, counting from the 5′ terminus of the antisense strand, nucleotides 1, 3, 5, 7, 8, 9, 11, 12, 13, 15, 17, 19, 21, 22, and 23 are 2′-O-methyl nucleotides, nucleotides 2, 4, 6, 10, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and the nucleotides of the sense strand are modified such that, counting from the 5′ terminus of the sense strand, nucleotides 1, 2, 3, 4, 5, 6, 8, 12, 13, 14, 15, 16, 17, 18, 19, 20, and 21 are 2′-O-methyl nucleotides, nucleotides 7, 9, 10, and 11 are 2′-fluoro nucleotides, the first two internucleoside linkages at the 5′ terminus are phosphorothioate internucleoside linkages, and each other internucleoside linkage is a phosphodiester internucleoside linkage. In such embodiments, the antisense strand has the modification pattern of Pattern XVII and the sense strand has the modification pattern of Pattern XIX.

In some embodiments, the compound includes an antisense strand and a sense strand hybridized to form a double stranded nucleic acid, wherein the antisense strand and the sense strand are not covalently linked, i.e., the antisense strand and sense strand from an siRNA, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and wherein the nucleotides of the sense strand are modified such that, counting from the 5′ terminus of the sense strand, nucleotides 1 and 2 are 2′-O-methoxyethyl nucleotides, nucleotides 3, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern V and the sense strand has the modification pattern of Pattern XX.

In some embodiments, the compound includes an antisense strand and a sense strand hybridized to form a double stranded nucleic acid, wherein the antisense strand and the sense strand are not covalently linked, i.e., the antisense strand and sense strand from an siRNA, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and wherein the nucleotides of the sense strand are modified such that, counting from the 5′ terminus of the sense strand, nucleotides 2 and 3 are 2′-O-methoxyethyl nucleotides, nucleotides 1, 4, 6, 8, 12, 14, 16, 18, 19, 20, and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern V and the sense strand has the modification pattern of Pattern XXI.

In some embodiments, the compound includes an antisense strand and a sense strand hybridized to form a double stranded nucleic acid, wherein the antisense strand and the sense strand are not covalently linked, i.e., the antisense strand and sense strand from an siRNA, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and wherein the nucleotides of the sense strand are modified such that, counting from the 5′ terminus of the sense strand, nucleotides 2, 3, 19 and 20 are 2′-O-methoxyethyl nucleotides, nucleotides 1, 4, 6, 8, 12, 14, 16, 18, and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern V and the sense strand has the modification pattern of Pattern XXII.

In some embodiments, the compound includes an antisense strand and a sense strand hybridized to form a double stranded nucleic acid, wherein the antisense strand and the sense strand are not covalently linked, i.e., the antisense strand and sense strand from an siRNA, wherein the antisense strand is 23 nucleotides in length and wherein the nucleotides of the antisense strand are modified such that, counting from the 5′ terminus of the antisense strand, nucleotides 1, 3, 5, 7, 9, 11, 13, 15, 17, 19, 21, 22, and 23 are 2′-O-methyl nucleotides, nucleotides 2, 4, 6, 8, 10, 12, 14, 16, 18, and 20 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 21 nucleotides in length and wherein the nucleotides of the sense strand are modified such that, counting from the 5′ terminus of the sense strand, nucleotides 1, 2, 3, and 4 are 2′-O-methoxyethyl nucleotides, nucleotides 6, 8, 12, 14, 16, 18, 19, 20 and 21 are 2′-O-methyl nucleotides, nucleotides 5, 7, 9, 10, 11, 13, 15, and 17 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern V and the sense strand has the modification pattern of Pattern XXIV.

In some embodiments, the compound includes an antisense strand and a sense strand hybridized to form a double stranded nucleic acid, wherein the antisense strand and the sense strand are not covalently linked, i.e., the antisense strand and sense strand from an siRNA, wherein the antisense strand is 21 nucleotides in length and wherein the nucleotides of the antisense strand are modified such that, counting from the 5′ terminus of the antisense strand, nucleotides 1, 3, 5, 7, 9, 11, 13, 15, 17, 19, 20, and 21 are 2′-O-methyl nucleotides, nucleotides 2, 4, 6, 8, 10, 12, 14, 16, 18 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 19 nucleotides in length and wherein the nucleotides of the sense strand are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern IV and the sense strand has the modification pattern of Pattern XXIV.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 21 nucleotides in length and the nucleotides of the antisense strand are modified such that, counting from the 5′ terminus of the antisense strand, nucleotides 1, 3, 5, 7, 9, 11, 13, 15, 17, 18, 19, 20, and 21 are 2′-O-methyl nucleotides, nucleotides 2, 4, 6, 8, 10, 12, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 19 nucleotides in length and wherein the nucleotides of the sense strand are modified such that, counting from the 5′ terminus of the sense strand, nucleotides 5, 7, 9, 11, 13, and 15 are 2′-fluoro nucleotides and nucleotides 1, 2, 3, 4, 6, 8, 10, 12, 14, 16, 17, 18, and 19 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern IV-1 and the sense strand has the modification pattern of Pattern XXV.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 21 nucleotides in length and the nucleotides of the antisense strand are modified such that, counting from the 5′ terminus of the antisense strand, nucleotides 1, 3, 4, 5, 7, 8, 9, 11, 13, 15, 17, 18, 19, 20, and 21 are 2′-O-methyl nucleotides, nucleotides 2, 6, 10, 12, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 19 nucleotides in length and wherein the nucleotides of the sense strand are modified such that, counting from the 5′ terminus of the sense strand, nucleotides 5, 8, 9, 11, and 13 are 2′-fluoro nucleotides and nucleotides 1, 2, 3, 4, 6, 7, 10, 12, 14, 15, 16, 17, 18, and 19 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern IV-2 and the sense strand has the modification pattern of Pattern XXVI.

In some embodiments, the compound includes an antisense strand and a sense strand hybridized to form a double stranded nucleic acid, wherein the antisense strand and sense strand are not covalently linked, i.e., the antisense strand and sense strand form an siRNA, wherein the antisense strand is 21 nucleotides in length and the nucleotides of the antisense strand are modified such that, counting from the 5′ terminus of the antisense strand, nucleotides 1, 3, 4, 5, 7, 8, 9, 10, 11, 12, 13, 15, 17, 18, 19, 20, and 21 are 2′-O-methyl nucleotides, nucleotides 2, 6, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and wherein the sense strand is 19 nucleotides in length and wherein the nucleotides of the sense strand are modified such that, counting from the 5′ terminus of the sense strand, nucleotides 5, 7, 8, and 9 are 2′-fluoro nucleotides and nucleotides 1, 2, 3, 4, 6, 10, 11, 12, 13, 14, 15, 16, 17, 18, and 19 are 2′-O-methyl nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage. In such embodiments, the antisense strand has the modification pattern of Pattern IV-3 and the sense strand has the modification pattern of Pattern XXVII.

In some embodiments, the modified double stranded nucleic acid is selected from a siRNA in Table 2, Table 8, Table 9 or Table 11.

In some embodiments, the antisense strand comprises a modified nucleic acid sequence selected from any one of the modified sequences listed in SEQ ID NO: 876-1166.

In some embodiments, the antisense strand is identical to a nucleic acid sequence selected from any one of the modified sequences listed in SEQ ID NO: 876-1166.

In some embodiments, the antisense strand comprises a modified nucleic acid sequence selected from any one of the modified sequences listed in Table 8, Table 9, or Table 11.

In some embodiments, the antisense strand is identical to a modified nucleic acid sequence selected from any one of the sequences listed in Table 8, Table 9, or Table 11.

In some embodiments, the antisense strand comprises or consists of a modified nucleic acid sequence that differs by 0 or 1 nucleotide selected from the group consisting of

(SEQ ID NO: 1268)
5′-X033U1027p001A007p001G004p000A007p000C004p000
A007p000A004p000U007p000A004p000A007p000A004p000
U007p000A004p000C007p000C004p000G007p000A004p000
G007p000G004p001A004p001A004-3′;
and
(SEQ ID NO: 1312)
5′-X033U1027p001C007p001A004p000A007p000U004p000
A007p000A004p000A007p000U004p000A007p000U004p000
C007p000C004p000A007p000A004p000A007p000C004p000
C007p000G004p001C004p001C004-3′,

wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, and G007 is 2′-fluoroguanosine.

In some embodiments, the antisense strand comprises or consists of a modified nucleic acid sequence that differs by 0 or 1 nucleotide selected from the group consisting of

(SEQ ID NO: 1346)
5′-X033U1027p001C007p001A004p000A007p000U004p000
A007p000A004p000A007p000U004p000A007p000U004p000
C007p000C004p000A007p000A004p000A007p000C004p000
C004p000G004p001C004p001C004-3′;
(SEQ ID NO: 1348)
5′-X033U1027p001C007p001A004p000A004p000U004p000
A007p000A004p000A004p000U004p000A007p000U004p000
C007p000C004p000A007p000A004p000A007p000C004p000
C004p000G004p001C004p001C004-3′;
and
(SEQ ID NO: 1352)
5′-X033U1027p001C007p001A004p000A004p000U004p000
A007p000A004p000A004p000U004p000A004p000U004p000
C004p000C004p000A007p000A004p000A007p000C004p000
C004p000G004p001C004p001C004-3′ ,

wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, G004 is 2′-O-methylguanosine, and G007 is 2′-fluoroguanosine.

In some embodiments, the sense strand comprises a modified nucleic acid sequence selected from any one of the modified sequences listed in SEQ ID NO: 585-875.

In some embodiments, the sense strand is identical to a modified nucleic acid sequence selected from any one of the modified sequences listed in SEQ ID NO: 585-875.

In some embodiments, the sense strand comprises a modified nucleic acid sequence selected from any one of the modified sequences listed in Table 8, Table 9, or Table 11.

In some embodiments, the sense strand is identical to a modified nucleic acid sequence selected from any one of the sequences listed in Table 8, Table 9, or Table 11.

In some embodiments, the sense strand comprises or consists of a modified nucleic acid sequence that differs by 0 or 1 nucleotide selected from the group consisting of

(SEQ ID NO: 1361)
5′-C007p001C004p001U007p000C004p000G007p000G004
p000U007p000A004p000U007p000U004p000U007p000A004
p000U007p000U004p000G007p000U004p000C007p001U004
p001A007-3′;
and
(SEQ ID NO: 1362)
5′-C007p001G004p001G007p000U004p000U007p000U004
p000G007p000G004p000A007p000U004p000A007p000U004
p000U007p000U004p000A007p000U004p000U007p001G004
p001A007-3′,

wherein p000 is a phosphate internucleoside linkage, p001 is a phosphorothioate internucleoside linkage, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, and G007 is 2′-fluoroguanosine.

In some embodiments, the sense strand comprises or consists of a modified nucleic acid sequence that differs by 0 or 1 nucleotide selected from the group consisting of

(SEQ ID NO: 1363)
5′-C004p001G004p001G004p000U004p000U007p000U004
p000G007p000G004p000A007p000U004p000A007p000U004
p000U007p000U004p000A007p000U004p000U004p001G004
p001A004-3′;
(SEQ ID NO: 1364)
5′-C004p001G004p001G004p000U004p000U007p000U004
p000G004p000G007p000A007p000U004p000A007p000U004
p000U007p000U004p000A004p000U004p000U004p001G004
p001A004-3′;
and
(SEQ ID NO: 1365)
5′-C004p001G004p001G004p000U004p000U007p000U004
p000G007p000G007p000A007p000U004p000A004p000U004
p000U004p000U004p000A004p000U004p000U004p001G004
p001U004-3′,

wherein p000 is a phosphate internucleoside linkage, p001 is a phosphorothioate internucleoside linkage, C004 is 2′-O-methylcytidine, A004 is 2′-O-methyladenosine, A007 is 2′-fluoroadenosine, U004 is 2′-O-methyluridine, U007 is 2′-fluorouridine, G004 is 2′-O-methylguanosine, and G007 is 2′-fluoroguanosine.

In some embodiments, the double stranded nucleic acid comprises
(i) an antisense strand comprising or consisting of the modified nucleic acid sequence
5′-X033U1027p001A007p001G004p000A007p000C004p000A007p000A004p000U007p000A004p000A007p000A004p000U007p000A004p000C007p000C004p000G007p000A004p000G007p000G004p001A004p001A004-3′ (SEQ ID NO: 1268); and a sense strand comprising or consisting of the modified nucleic acid sequence 5′-C007p001C004p001U007p000C004p000G007p000G004p000U007p000A004p000U007p000U 004p000U007p000A004p000U007p000U004p000G007p000U004p000C007p001U004p001A007-3′ (SEQ ID NO: 1361); or
(ii) an antisense strand comprising or consisting of the modified nucleic acid sequence
5′-X033U1027p001C007p001A004p000A007p000U004p000A007p000A004p000A007p000U004p000A007p000U004p000C007p000C004p000A007p000A004p000A007p000C004p000C007p000G004p001C004p001C004-3′ (SEQ ID NO: 1312); and a sense strand comprising or consisting of the modified nucleic acid sequence 5′-C007p001G004p001G007p000U004p000U007p000U004p000G007p000G004p000A007p000U 004p000A007p000U004p000U007p000U004p000A007p000U004p000U007p001G004p001A007-3′ (SEQ ID NO: 1362),
wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, and G007 is 2′-fluoroguanosine.

In some embodiments, the double stranded nucleic acid comprises
(i) an antisense strand comprising or consisting of the modified nucleic acid sequence
5′-X033U1027p001C007p001A004p000A007p000U004p000A007p000A004p000A007p000U004p000A007p000U004p000C007p000C004p000A007p000A004p000A007p000C004p000C004p000G004p001C004p001C004-3′ (SEQ ID NO: 1346); and a sense strand comprising or consisting of the modified nucleic acid sequence 5′-C00 G004G004p001G004p000U004p000U007p000U004p000G007p000G004p000A007p000U 004p000A007p000U004p000U007p000U004p000A007p000U004p000U004p001G004p001A004-3′ (SEQ ID NO: 1363);
(ii) an antisense strand comprising or consisting of the modified nucleic acid sequence
5′-X033U1027p001C007p001A004p000A004p000U004p000A007p000A004p000A004p000U004p000A007p000U004p000C007p000C004p000A007p000A004p000A007p000C004p000C004p000G004p001C004p001C004-3′ (SEQ ID NO: 1348); and a sense strand comprising or consisting of the modified nucleic acid sequence 5′-C004p001G004p001G004p000U004p000U007p000U004p000G004p000G007p000A007p000U 004p000A007p000U004p000U007p000U004p000A004p000U004p000U004p001G004p001A004-3′ (SEQ ID NO: 1364); or
(iii) an antisense strand comprising or consisting of the modified nucleic acid sequence
5′-X033U1027p001C007p001A004p000A004p000U004p000A007p000A004p000A004p000U004p000A004p000U004p000C004p000C004p000A007p000A004p000A007p000C004p000C004p000G004p001C004p001C004-3′ (SEQ ID NO: 1352), and a sense strand comprising or consisting of the modified nucleic acid sequence 5′-C004p001G004p001G004p000U004p000U007p000U004p000G007p000G007p000A007p000U 004p000A004p000U004p000U004p000U004p000A004p000U004p000U004p001G004p001U004-3′ (SEQ ID NO: 1365),
wherein p000 is a phosphate internucleoside linkage, p001 is a phosphorothioate internucleoside, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, and G007 is 2′-fluoroguanosine.

In embodiments, A is selected from a siRNA in Table 8, Table 9 or Table 11.

### Linkers, Fatty Acid Moieties and Conjugated Compounds

In some embodiments, a dsRNAi agent provided herein includes a covalently linked ligand. In some embodiments, a compound provided herein includes a ligand covalently linked to the antisense strand. In some embodiments, a compound provided herein comprises a ligand covalently linked to the sense strand. In some embodiments, the ligand comprises a fatty acid moiety with one or more long chain fatty acids (LFCA). In some embodiments, the ligand comprises a fatty acid moiety with one or more medium chain fatty acids (MCFA). In some embodiments, the ligand comprises a fatty acid moiety with one or more medium chain fatty acids and one or more long chain fatty acids. In some embodiments, the ligand comprises a multibranched fatty acid moiety.

In some embodiments, the use of a fatty acid moiety facilitates delivery of the double stranded nucleic acid into a cell, such as a skeletal muscle cell.

In some embodiments, the fatty acid moiety is capable of facilitating systemic delivery into one or more cells, e.g., one or more skeletal muscle cells. Without wishing to be bound by any theory, it is hypothesized that conjugation of the oligonucleotides with fatty acids may increase the albumin binding affinity of the oligonucleotides, enhancing their ability to cross the endothelial barrier and improving their functional uptake into muscles, thereby increasing the oligonucleotide potency in vivo.

In some embodiments, the fatty acid moiety has or is capable of having binding activity in a cell, e.g., in a skeletal muscle cell. In some embodiments, the fatty acid moiety has or is capable of having lipoprotein binding activity in a cell, e.g., in a skeletal muscle cell. In some embodiments, the fatty acid moiety has or is capable of having an albumin binding activity, and thus can instill in the conjugate the ability to bind albumin. The fatty acid moiety can include any fatty acid moiety suitable for binding into a fatty acid pocket of albumin.

In some embodiments, the use of a fatty acid moiety provides increased biodistribution within one or more target tissues, e.g., muscle and/or nerve. In some embodiments, the one or more target tissues are disease-relevant tissues. In some embodiments, the one or more target tissues is selected from tissues of brain, spinal cord, muscle, spleen, lung, heart, liver, bladder, and kidney.

In some embodiments, the one or more target tissues is selected from the group consisting of prefrontal cortex, cerebellum, and cerebrum; cervical, thoracic and lumbar in spinal cord; heart, bicep, tricep, semitendinosus, platysma, gluteus, tibialis anterior, quadriceps, and/or gastrocnemius.

In some embodiments, the use of a fatty acid moiety enhances potency of the double stranded nucleic acids in muscle. In some embodiments, the fatty acid moiety improves the suppressing activity of DMPK gene expression in skeletal muscles, heart, and the like. dsRNAi agents comprising a siRNA as the double stranded nucleic acid have an improved DMPK inhibition effect as compared to a control, e.g., said dsRNAi without the fatty acid moiety.

In some embodiments, the fatty acid moiety is capable of increasing the in-vivo half-life and/or is capable of changing the biodistribution properties of the double stranded nucleic acid within one or more target tissues, e.g., muscle and/or nerve. In some embodiments, the one or more target tissues are disease-relevant tissues. In some embodiments, the one or more target tissues is selected from tissues of brain, spinal cord, muscle, spleen, lung, heart, liver, bladder, and kidney.

In some embodiments, the one or more target tissues is selected from the group consisting of prefrontal cortex, cerebellum, and cerebrum; cervical, thoracic and lumbar in spinal cord; heart, bicep, tricep, semitendinosus, platysma, gluteus, tibialis anterior, quadriceps, and/or gastrocnemius.

In some embodiments, the use of a fatty acid moiety improves the stability, bioavailability, biodistribution, and/or cellular uptake of the double stranded nucleic acid as compared to a control, e.g., the double stranded nucleic acid without the fatty acid moiety.

In some embodiments, a dsRNAi provided herein comprises
(i) a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length; and
(ii) a fatty acid moiety capable of facilitating delivery of the double stranded nucleic acid into a cell;
wherein the double stranded nucleic acid is linked to the fatty acid moiety via a linker.

In some embodiments, the fatty acid moiety has binding activity in a cell, for example, an albumin binding activity, which is capable of increasing the in-vivo half-life of the compound and/or is capable of changing or increasing the biodistribution properties of the dsRNAi within one or more target tissues.

In some embodiments, provided herein is a dsRNAi agent, or a pharmaceutically acceptable salt thereof, of formula (I)

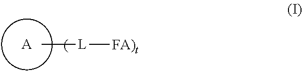

wherein:
A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
L is a linker;
FA is a fatty acid moiety capable of facilitating delivery of the double stranded nucleic acid into a cell, e.g., a skeletal muscle cell; and
t is an integer from 1 to 5.

In some embodiments, t is 1, 2 or 3. In some embodiments, t is 1. In some embodiments, t is 2. In embodiment, t is 3. In some embodiments, t is 4. In embodiment t is 5.

Additional embodiments of the double stranded nucleic acid A, including modified double stranded nucleic acids, are disclosed hereinbefore and throughout the application.

Additional embodiments of the linker L and fatty acid moiety FA are disclosed hereinafter and throughout the application.

### Linker

In some embodiments, the linker is covalently attached to the sense strand or antisense strand of the double stranded nucleic acid. In some embodiments, the linker is covalently attached to the sense strand of the double stranded nucleic acid. In some embodiments, the linker is covalently attached to the 3′ terminus of the sense strand, e.g., via the 3′-terminal hydroxyl group of the sense strand. In some embodiments, the linker is covalently attached to the 5′ terminus of the sense strand, e.g., via the 5′-terminal hydroxyl group of the sense strand.

In some embodiments, the linker is attached to the 3′ carbon of a 3′ terminal nucleotide of the double stranded oligonucleotide. In some embodiments, the linker is attached to the 3′ carbon of a 3′ terminal nucleotide of the sense strand of the double stranded nucleic acid.

In some embodiments, the linker is attached to the 5′ carbon of a 5′ terminal nucleotide of the double stranded oligonucleotide. In some embodiments, the linker is attached to the 5′ carbon of a 5′ terminal nucleotide of the sense strand of the double stranded nucleic acid.

In some embodiments, the linker is attached to a 2′ carbon of a nucleotide of the double stranded oligonucleotide. In some embodiments, the 2′ carbon is the 2′ carbon of an internal nucleotide.

In some embodiments, the linker is attached to a nucleobase of the double stranded nucleic acid.

In some embodiments, the linker is directly attached to the double stranded nucleic acid or indirectly through one or more groups such as —O—, —NH—, —S—, —C(O)—, —C(O)O—, —OC(O)—, —C(O)NH—, —NHC(O)—, —OP(O)(OH)O—, and —OP(S)(OH)O—. In some embodiments, the linker is directly attached to the double stranded nucleic acid or indirectly through one or more groups such as —O—, —OP(O)(OH)O—, and —OP(S)(OH)O—.

In some embodiments, the linker comprises a functional group selected from the group consisting of —NH—, —O—, —S—, —C(O)—, —NHC(O)—, —NHC(O)NH—, —C(O)O—, —OC(O)—, —C(O)NH—, —OP(O)(OH)O—, —OP(S)(OH)O—, substituted or unsubstituted C₁-C₂₀ alkylene, substituted or unsubstituted C₂-C₂₀ alkenylene, substituted or unsubstituted 2 to 20 membered heteroalkylene, substituted or unsubstituted C₃-C₁₀ cycloalkylene, substituted or unsubstituted 2 to 20 membered heterocycloalkylene, substituted or unsubstituted C₆-C₁₂ arylene or substituted or unsubstituted 5 to 12 membered heteroarylene.

In some embodiments, the linker comprises a substituted or unsubstituted C₁-C₂₀ alkylene, substituted or unsubstituted C₂-C₂₀ alkenylene, or substituted or unsubstituted 2 to 20 membered heteroalkylene.

In some embodiments, the C₁-C₂₀ alkylene, C₂-C₂₀ alkenylene, 2 to 20 membered heteroalkylene, C₃-C₁₀ cycloalkylene, 2 to 20 membered heterocycloalkylene, C₆-C₁₂ arylene, and 5 to 12 membered heteroarylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, the linker comprises a substituted or unsubstituted C₁-C₂₀ alkylene, substituted or unsubstituted C₂-C₂₀ alkenylene, or substituted or unsubstituted 2 to 20 membered heteroalkylene, wherein the C₁-C₂₀ alkylene, C₂-C₂₀ alkenylene, and 2 to 20 membered heteroalkylene, when substituted, are each independently substituted with 1, 2, 3, 4 or 5 substituents independently selected from the group consisting of hydroxyl, oxo, carboxyl, mercapto, and amino.

In some embodiments, the linker comprises a group selected from the group consisting of

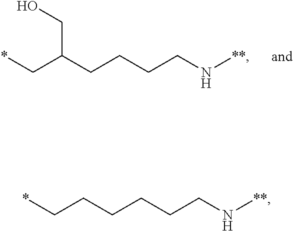

wherein * indicates the point of attachment to the double stranded nucleic acid and ** indicates the point of attachment to the fatty acid motif.

In some embodiments, the linker is selected from

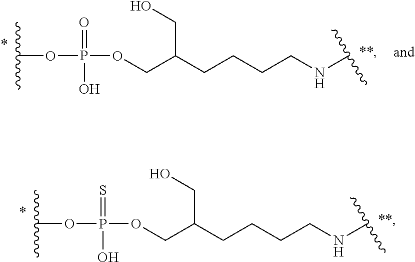 [SMILES: *NCCCCC(CO)COP(=O)(O)O* ; *NCCCCC(CO)COP(O)(=S)O*]

wherein * indicates the point of attachment to the double stranded nucleic acid and ** indicates the point of attachment to the fatty acid moiety. In some embodiments, * indicates the point of attachment to a 3′ carbon of a terminal 3′ nucleotide or to a 5′ carbon of a terminal 5′ nucleotide of the sense strand of the double stranded nucleic acid.

In some embodiments, the linker is selected from

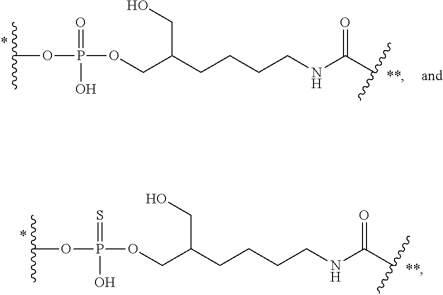 [SMILES: *OP(=O)(O)OCC(CO)CCCCNC(*)=O ; *OP(O)(=S)OCC(CO)CCCCNC(*)=O]

wherein * indicates the point of attachment to the double stranded nucleic acid and ** indicates the point of attachment to the fatty acid moiety. In some embodiments, * indicates the point of attachment to a 3′ carbon of a terminal 3′ nucleotide or to a 5′ carbon of a terminal 5′ nucleotide of the sense strand of the double stranded nucleic acid.

In some embodiments, the linker is selected from

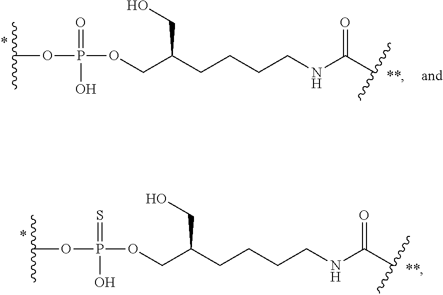 [SMILES: *OP(=O)(O)OC[C@@H](CO)CCCCNC(*)=O ; *OP(O)(=S)OC[C@@H](CO)CCCCNC(*)=O]

wherein * indicates the point of attachment to the double stranded nucleic acid and ** indicates the point of attachment to the fatty acid moiety. In some embodiments, * indicates the point of attachment to a 3′ carbon of a terminal 3′ nucleotide or to a 5′ carbon of a terminal 5′ nucleotide of the sense strand of the double stranded nucleic acid.

In some embodiments, the linker is selected from

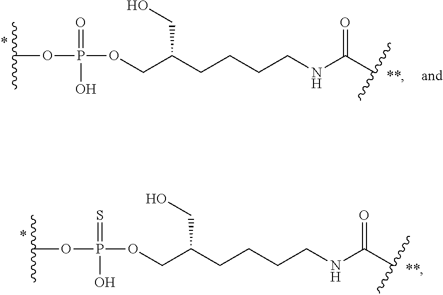 [SMILES: *OP(=O)(O)OC[C@H](CO)CCCCNC(*)=O ; *OP(O)(=S)OC[C@H](CO)CCCCNC(*)=O]

wherein * indicates the point of attachment to the double stranded nucleic acid and ** indicates the point of attachment to the fatty acid moiety. In some embodiments, * indicates the point of attachment to a 3′ carbon of a terminal 3′ nucleotide or a 5′ carbon of a terminal 5′ nucleotide of the sense strand of the double stranded nucleic acid.

In some embodiments, the linker is a group of formula

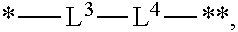

wherein
L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)O—, substituted or unsubstituted C₁-C₂₀ alkylene, substituted or unsubstituted C₂-C₂₀ alkenylene, substituted or unsubstituted 2 to 20 membered heteroalkylene, substituted or unsubstituted C₃-C₁₀ cycloalkylene, substituted or unsubstituted 2 to 20 membered heterocycloalkylene, substituted or unsubstituted C₆-C₁₂ arylene or substituted or unsubstituted 5 to 12 membered heteroarylene, and
wherein * indicates the point of attachment to the double stranded nucleic acid and ** indicates the point of attachment to the fatty acid moiety.

In some embodiments, L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, —OP(S)(O)O—, substituted or unsubstituted C₁-C₁₂ alkylene, substituted or unsubstituted C₂-C₁₂ alkenylene, or substituted or unsubstituted 2 to 20 membered heteroalkylene.

In some embodiments, the C₁-C₂₀ alkylene, C₁-C₁₂ alkylene, C₂-C₂₀ alkenylene, C₂-C₁₂ alkenylene, 2 to 20 membered heteroalkylene, C₃-C₁₀ cycloalkylene, 2 to 20 membered heterocycloalkylene, C₆-C₁₂ arylene, and 5 to 12 membered heteroarylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, the C₁-C₂₀ alkylene, C₁-C₁₂ alkylene, C₂-C₂₀ alkenylene, C₂-C₁₂ alkenylene, 2 to 20 membered heteroalkylene, C₃-C₁₀ cycloalkylene, 2 to 20 membered heterocycloalkylene, C₆-C₁₂ arylene, and 5 to 12 membered heteroarylene are each independently unsubstituted or substituted with 1, 2, 3, 4 or 5 substituents independently selected from the group consisting of hydroxyl, oxo, carboxyl, mercapto, and amino.

In some embodiments, one L³ is attached to a 3′ carbon of the double stranded oligonucleotide. In some embodiments, one L³ is attached to the 3′ carbon of a 3′ terminal nucleotide of the double stranded oligonucleotide. In some embodiments, one L³ is attached to the 3′ carbon of a 3′ terminal nucleotide of the sense strand of the double stranded oligonucleotide.

In some embodiments, one L³ is attached to a 5′ carbon of the double stranded oligonucleotide. In some embodiments, one L³ is attached to the 5′ carbon of a 5′ terminal nucleotide of a double stranded oligonucleotide. In some embodiments, one L³ is attached to the 5′ carbon of a 5′ terminal nucleotide of the sense strand of the double stranded oligonucleotide.

In some embodiments, one L³ is attached to a 2′ carbon of a nucleotide of the double-stranded oligonucleotide. In some embodiments, the 2′ carbon is the 2′ carbon of an internal nucleotide.

In some embodiments, one L³ is attached to a nucleobase of the double stranded oligonucleotide.

In some embodiments, t is 1, and L³ is attached to a 3′ carbon of a 3′ terminal nucleotide of the sense strand of the double stranded nucleic acid.

In some embodiments, L³ is independently a bond, —NH—, —O—, —S—, —C(O)—, —NHC(O)—, —NHC(O)NH—, —C(O)O—, —OC(O)—, —C(O)NH—, —OPO₂—O—, —OP(S)(OH)—O—, substituted or unsubstituted C₁-C₂₀ alkylene or substituted or unsubstituted 2 to 20 membered heteroalkylene. In some embodiments, the C₁-C₂₀ alkylene and 2 to 20 membered heteroalkylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is independently a bond, —NH—, —O—, —S—, —C(O)—, —NHC(O)—, —NHC(O)NH—, —C(O)O—, —OC(O)—, —C(O)NH—, —OPO₂—O—, —OP(S)(OH)—O—, substituted or unsubstituted C₁-C₂₀ alkylene or substituted or unsubstituted 2 to 20 membered heteroalkylene. In some embodiments, the C₁-C₂₀ alkylene and 2 to 20 membered heteroalkylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L³ is independently a bond. In some embodiments, L³ is independently —NH—. In some embodiments, L³ is independently —O—. In some embodiments, L³ is independently —S—. In some embodiments, L³ is independently —C(O)—. In some embodiments, L³ is independently —NHC(O)—. In some embodiments, L³ is independently —NHC(O)NH—. In some embodiments, L³ is independently —C(O)O—. In some embodiments, L³ is independently —OC(O)—. In some embodiments, L³ is independently —C(O)NH—. In some embodiments, L³ is independently —OPO₂—O—. In some embodiments, L³ is independently —OP(S)(OH)—O—. In some embodiments, L³ is independently substituted or unsubstituted C₁-C₂₀ alkylene. In some embodiments, L³ is independently substituted or unsubstituted 2 to 20 membered heteroalkylene. In some embodiments, the C₁-C₂₀ alkylene and 2 to 20 membered heteroalkylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L³ is independently substituted or unsubstituted alkylene, e.g., C₁-C₂₀, C₁-C₁₂, C₁-C₈, C₁-C₆, C₁-C₄, or C₁-C₂. In some embodiments, L³ is independently substituted alkylene, e.g., C₁-C₂₀, C₁-C₁₂, C₁-C₈, C₁-C₆, C₁-C₄, or C₁-C₂. In some embodiments, L³ is independently unsubstituted alkylene, e.g., C₁-C₂₀, C₁-C₁₂, C₁-C₈, C₁-C₆, C₁-C₄, or C₁-C₂. In some embodiments, L³ is independently substituted or unsubstituted C₁-C₂₀ alkylene. In some embodiments, L³ is independently substituted C₁-C₂₀ alkylene. In some embodiments, L³ is independently unsubstituted C₁-C₂₀ alkylene. In some embodiments, L³ is independently substituted or unsubstituted C₁-C₁₂ alkylene. In some embodiments, L³ is independently substituted C₁-C₁₂ alkylene. In some embodiments, L³ is independently unsubstituted C₁-C₁₂ alkylene. In some embodiments, L³ is independently substituted or unsubstituted C₁-C₈ alkylene. In some embodiments, L³ is independently substituted C₁-C₈ alkylene. In some embodiments, L³ is independently unsubstituted C₁-C₈ alkylene. In some embodiments, L³ is independently substituted or unsubstituted C₁-C₆ alkylene. In some embodiments, L³ is independently substituted C₁-C₆ alkylene. In some embodiments, L³ is independently unsubstituted C₁-C₆ alkylene. In some embodiments, L³ is independently substituted or unsubstituted C₁-C₄ alkylene. In some embodiments, L³ is independently substituted C₁-C₄ alkylene. In some embodiments, L³ is independently unsubstituted C₁-C₄ alkylene. In some embodiments, L³ is independently substituted or unsubstituted ethylene. In some embodiments, L³ is independently substituted ethylene. In some embodiments, L³ is independently unsubstituted ethylene. In some embodiments, L³ is independently substituted or unsubstituted methylene. In some embodiments, L³ is independently substituted methylene. In some embodiments, L³ is independently unsubstituted methylene. In some embodiments, the alkylene, e.g., C₁-C₂₀, C₁-C₁₂, C₁-C₈, C₁-C₆, C₁-C₄, or C₁-C₂ alkylene, is independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L³ is independently substituted or unsubstituted heteroalkylene, e.g., 2 to 20 membered, 2 to 12 membered, 2 to 8 membered, 2 to 6 membered, 4 to 6 membered, 2 to 3 membered, or 4 to 5 membered. In some embodiments, L³ is independently substituted heteroalkylene, e.g., 2 to 20 membered, 2 to 12 membered, 2 to 8 membered, 2 to 6 membered, 4 to 6 membered, 2 to 3 membered, or 4 to 5 membered. In some embodiments, L³ is independently unsubstituted heteroalkylene, e.g., 2 to 20 membered, 2 to 12 membered, 2 to 8 membered, 2 to 6 membered, 4 to 6 membered, 2 to 3 membered, or 4 to 5 membered. In some embodiments, L³ is independently substituted or unsubstituted 2 to 20 membered heteroalkylene. In some embodiments, L³ is independently substituted 2 to 20 membered heteroalkylene. In some embodiments, L³ is independently unsubstituted 2 to 20 membered heteroalkylene. In some embodiments, L³ is independently substituted or unsubstituted 2 to 8 membered heteroalkylene. In some embodiments, L³ is independently substituted 2 to 8 membered heteroalkylene. In some embodiments, L³ is independently unsubstituted 2 to 8 membered heteroalkylene. In some embodiments, L³ is independently substituted or unsubstituted 2 to 6 membered heteroalkylene. In some embodiments, L³ is independently substituted 2 to 6 membered heteroalkylene. In some embodiments, L³ is independently unsubstituted 2 to 6 membered heteroalkylene. In some embodiments, L³ is independently substituted or unsubstituted 4 to 6 membered heteroalkylene. In some embodiments, L³ is independently substituted 4 to 6 membered heteroalkylene. In some embodiments, L³ is independently unsubstituted 4 to 6 membered heteroalkylene. In some embodiments, L³ is independently substituted or unsubstituted 2 to 3 membered heteroalkylene. In some embodiments, L³ is independently substituted 2 to 3 membered heteroalkylene. In some embodiments, L³ is independently unsubstituted 2 to 3 membered heteroalkylene. In some embodiments, L³ is independently substituted or unsubstituted 4 to 5 membered heteroalkylene. In some embodiments, L³ is independently substituted 4 to 5 membered heteroalkylene. In some embodiments, L³ is independently unsubstituted 4 to 5 membered heteroalkylene. In some embodiments, the heteroalkylene, e.g., 2 to 20 membered, 2 to 12 membered, 2 to 8 membered, 2 to 6 membered, 4 to 6 membered, 2 to 3 membered, or 4 to 5 membered heteroalkylene, is independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is independently a bond. In some embodiments, L⁴ is independently —NH—. In some embodiments, L⁴ is independently —O—. In some embodiments, L⁴ is independently —S—. In some embodiments, L⁴ is independently —C(O)—. In some embodiments, L⁴ is independently —NHC(O)—. In some embodiments, L⁴ is independently —NHC(O)NH—. In some embodiments, L⁴ is independently —C(O)O—. In some embodiments, L⁴ is independently —OC(O)—. In some embodiments, L⁴ is independently —C(O)NH—. In some embodiments, L⁴ is independently —OPO₂—O—. In some embodiments, L⁴ is independently —OP(S)(OH)—O—. In some embodiments, L⁴ is independently substituted or unsubstituted C₁-C₂₀ alkylene. In some embodiments, L⁴ is independently substituted or unsubstituted 2 to 20 membered heteroalkylene. In some embodiments, the C₁-C₂₀ alkylene and 2 to 20 membered heteroalkylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is independently substituted or unsubstituted alkylene, e.g., C₁-C₂₀, C₁-C₁₂, C₁-C₈, C₁-C₆, C₁-C₄, or C₁-C₂. In some embodiments, L⁴ is independently substituted alkylene, e.g., C₁-C₂₀, C₁-C₁₂, C₁-C₈, C₁-C₆, C₁-C₄, or C₁-C₂. In some embodiments, L⁴ is independently unsubstituted alkylene, e.g., C₁-C₂₀, C₁-C₁₂, C₁-C₈, C₁-C₆, C₁-C₄, or C₁-C₂. In some embodiments, L⁴ is independently substituted or unsubstituted C₁-C₂₀ alkylene. In some embodiments, L⁴ is independently substituted C₁-C₂₀ alkylene. In some embodiments, L⁴ is independently unsubstituted C₁-C₂₀ alkylene. In some embodiments, L⁴ is independently substituted or unsubstituted C₁-C₁₂ alkylene. In some embodiments, L⁴ is independently substituted C₁-C₁₂ alkylene. In some embodiments, L⁴ is independently unsubstituted C₁-C₁₂ alkylene. In some embodiments, L⁴ is independently substituted or unsubstituted C₁-C₈ alkylene. In some embodiments, L⁴ is independently substituted C₁-C₈ alkylene. In some embodiments, L⁴ is independently unsubstituted C₁-C₈ alkylene. In some embodiments, L⁴ is independently substituted or unsubstituted C₁-C₆ alkylene. In some embodiments, L⁴ is independently substituted C₁-C₆ alkylene. In some embodiments, L⁴ is independently unsubstituted C₁-C₆ alkylene. In some embodiments, L⁴ is independently substituted or unsubstituted C₁-C₄ alkylene. In some embodiments, L⁴ is independently substituted C₁-C₄ alkylene. In some embodiments, L⁴ is independently unsubstituted C₁-C₄ alkylene. In some embodiments, L⁴ is independently substituted or unsubstituted ethylene. In some embodiments, L⁴ is independently substituted ethylene. In some embodiments, L⁴ is independently unsubstituted ethylene. In some embodiments, L⁴ is independently substituted or unsubstituted methylene. In some embodiments, L⁴ is independently substituted methylene. In some embodiments, L⁴ is independently unsubstituted methylene. In some embodiments, the alkylene, e.g., C₁-C₂₀, C₁-C₁₂, C₁-C₈, C₁-C₆, C₁-C₄, or C₁-C₂ alkylene, is independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is independently substituted or unsubstituted heteroalkylene, e.g., 2 to 20 membered, 2 to 12 membered, 2 to 8 membered, 2 to 6 membered, 4 to 6 membered, 2 to 3 membered, or 4 to 5 membered. In some embodiments, L⁴ is independently substituted heteroalkylene, e.g., 2 to 20 membered, 2 to 12 membered, 2 to 8 membered, 2 to 6 membered, 4 to 6 membered, 2 to 3 membered, or 4 to 5 membered. In some embodiments, L⁴ is independently unsubstituted heteroalkylene, e.g., 2 to 20 membered, 2 to 12 membered, 2 to 8 membered, 2 to 6 membered, 4 to 6 membered, 2 to 3 membered, or 4 to 5 membered. In some embodiments, L⁴ is independently substituted or unsubstituted 2 to 20 membered heteroalkylene. In some embodiments, L⁴ is independently substituted 2 to 20 membered heteroalkylene. In some embodiments, L⁴ is independently unsubstituted 2 to 20 membered heteroalkylene. In some embodiments, L⁴ is independently substituted or unsubstituted 2 to 8 membered heteroalkylene. In some embodiments, L⁴ is independently substituted 2 to 8 membered heteroalkylene. In some embodiments, L⁴ is independently unsubstituted 2 to 8 membered heteroalkylene. In some embodiments, L⁴ is independently substituted or unsubstituted 2 to 6 membered heteroalkylene. In some embodiments, L⁴ is independently substituted 2 to 6 membered heteroalkylene. In some embodiments, L⁴ is independently unsubstituted 2 to 6 membered heteroalkylene. In some embodiments, L⁴ is independently substituted or unsubstituted 4 to 6 membered heteroalkylene. In some embodiments, L⁴ is independently substituted 4 to 6 membered heteroalkylene. In some embodiments, L⁴ is independently unsubstituted 4 to 6 membered heteroalkylene. In some embodiments, L⁴ is independently substituted or unsubstituted 2 to 3 membered heteroalkylene. In some embodiments, L⁴ is independently substituted 2 to 3 membered heteroalkylene. In some embodiments, L⁴ is independently unsubstituted 2 to 3 membered heteroalkylene. In some embodiments, L⁴ is independently substituted or unsubstituted 4 to 5 membered heteroalkylene. In some embodiments, L⁴ is independently substituted 4 to 5 membered heteroalkylene. In some embodiments, L⁴ is independently unsubstituted 4 to 5 membered heteroalkylene. In some embodiments, the heteroalkylene, e.g., 2 to 20 membered, 2 to 12 membered, 2 to 8 membered, 2 to 6 membered, 4 to 6 membered, 2 to 3 membered, or 4 to 5 membered heteroalkylene, is independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L³ is independently

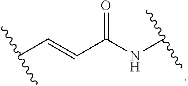 [SMILES: */C=C/C(=O)N*]

In some embodiments, L³ is independently a bond. In some embodiments, L³ is independently —OPO₂—O—. In some embodiments, L³ is independently —OP(S)(OH)—O—. In some embodiments, L³ is independently —O—.

In some embodiments, L⁴ is independently substituted or unsubstituted C₁-C₂₀ alkylene or substituted or unsubstituted 2 to 20 membered heteroalkylene.

In some embodiments, L⁴ is independently substituted or unsubstituted C₁-C₂₀ alkylene or substituted or unsubstituted 2 to 20 membered heteroalkylene, wherein the C₁-C₂₀ alkylene and 2 to 20 membered heteroalkylene, when substituted, are each independently substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is independently -L⁷-NH—C(O)— or -L⁷-C(O)—NH—. In some embodiments, L⁷ is independently substituted or unsubstituted alkylene, e.g., C₁-C₂₀, C₁-C₁₂, C₁-C₈, C₁-C₆, C₁-C₄, or C₁-C₂. In some embodiments, L⁷ is independently substituted alkylene, e.g., C₁-C₂₀, C₁-C₁₂, C₁-C₈, C₁-C₆, C₁-C₄, or C₁-C₂. In some embodiments, L⁷ is independently unsubstituted alkylene, e.g., C₁-C₂₀, C₁-C₁₂, C₁-C₈, C₁-C₆, C₁-C₄, or C₁-C₂. In some embodiments, the alkylene, e.g., C₁-C₂₀, C₁-C₁₂, C₁-C₈, C₁-C₆, C₁-C₄, or C₁-C₂ alkylene, is independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C1-C4 alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is independently -L⁷-NH—C(O)— or -L⁷-C(O)—NH—, wherein L⁷ is substituted or unsubstituted C₁-C₂₀ alkylene. In some embodiments, the C1-C₂₀ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is independently -L⁷-NH—C(O)— or -L⁷-C(O)—NH—, wherein L⁷ is substituted or unsubstituted C₁-C₂₀ alkylene, wherein the C₁-C₂₀ alkylene, when substituted, is substituted with 1, 2, 3, 4 or 5 substituents independently selected from the group consisting of hydroxyl, oxo, carboxyl, mercapto, and amino.

In some embodiments, L⁷ is independently substituted C₁-C₂₀ alkylene. In some embodiments, L⁷ is independently hydroxy(OH)-substituted C₁-C₂₀ alkylene. In some embodiments, L⁷ is independently hydroxymethyl-substituted C₁-C₂₀ alkylene. In some embodiments, L⁷ is independently unsubstituted C₁-C₂₀ alkylene. In some embodiments, L⁷ is independently substituted or unsubstituted C₁-C₁₂ alkylene. In some embodiments, L⁷ is independently substituted C₁-C₁₂ alkylene. In some embodiments, L⁷ is independently hydroxy(OH)-substituted C₁-C₁₂ alkylene. In some embodiments, L⁷ is independently hydroxymethyl-substituted C₁-C₁₂ alkylene. In some embodiments, L⁷ is independently unsubstituted C₁-C₁₂ alkylene. In some embodiments, L⁷ is independently substituted or unsubstituted C₁-C₈ alkylene. In some embodiments, L⁷ is independently substituted C₁-C₈ alkylene. In some embodiments, L⁷ is independently hydroxy(OH)-substituted C₁-C₈ alkylene. In some embodiments, L⁷ is independently hydroxymethyl-substituted C₁-C₈ alkylene. In some embodiments, L⁷ is independently unsubstituted C₁-C₈ alkylene. In some embodiments, L⁷ is independently substituted or unsubstituted C₁-C₆ alkylene. In some embodiments, L⁷ is independently substituted C₁-C₆ alkylene. In some embodiments, L⁷ is independently hydroxy(OH)-substituted C₁-C₆ alkylene. In some embodiments, L⁷ is independently hydroxymethyl-substituted C₁-C₆ alkylene. In some embodiments, L⁷ is independently unsubstituted C₁-C₆ alkylene. In some embodiments, L⁷ is independently substituted or unsubstituted C₁-C₄ alkylene. In some embodiments, L⁷ is independently substituted C₁-C₄ alkylene. In some embodiments, L⁷ is independently hydroxy(OH)-substituted C₁-C₄ alkylene. In some embodiments, L⁷ is independently hydroxymethyl-substituted C₁-C₄ alkylene. In some embodiments, L⁷ is independently unsubstituted C₁-C₄ alkylene. In some embodiments, L⁷ is independently substituted or unsubstituted C₁-C₂ alkylene. In some embodiments, L⁷ is independently substituted C₁-C₂ alkylene. In some embodiments, L⁷ is independently hydroxy(OH)-substituted C₁-C₂ alkylene. In some embodiments, L⁷ is independently hydroxymethyl-substituted C₁-C₂ alkylene. In some embodiments, L⁷ is independently unsubstituted C₁-C₂ alkylene.

In some embodiments, L⁴ is independently -L⁷-NH—C(O)—, wherein L⁷ is substituted C₁-C₁₂ alkylene, wherein the C₁-C₁₂ alkylene is substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is independently -L⁷-NH—C(O)— or -L⁷-C(O)—NH—; and L⁷ is independently substituted or unsubstituted C₃-C₈ alkylene. In some embodiments, L⁴ is independently -L⁷-NH—C(O)— or -L⁷-C(O)—NH—; and L⁷ is independently substituted C₃-C₈ alkylene. In some embodiments, the C₃-C₈ alkylene is substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In some embodiments, L⁴ is independently -L⁷-NH—C(O)— or -L⁷-C(O)—NH—; and L⁷ is independently hydroxy(OH)-substituted C₃-C₈ alkylene. In some embodiments, L⁴ is independently -L⁷-NH—C(O)— or -L⁷-C(O)—NH—; and L⁷ is independently hydroxymethyl-substituted C₃-C₈ alkylene. In some embodiments, L⁴ is independently -L⁷-NH—C(O)— or -L⁷-C(O)—NH—; and L⁷ is independently unsubstituted C₃-C₈ alkylene.

In some embodiments, L⁴ is independently -L⁷-NH—C(O)— or -L⁷-C(O)—NH—; and L⁷ is independently substituted or unsubstituted C₅-C₈alkylene. In some embodiments, L⁴ is independently -L⁷-NH—C(O)— or -L⁷-C(O)—NH—; and L⁷ is independently substituted C₅-C₈ alkylene. In some embodiments, the C₅-C₈alkylene is substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In some embodiments, L⁴ is independently -L⁷-NH—C(O)— or -L⁷-C(O)—NH—; and L⁷ is independently hydroxy(OH)-substituted C₅-C₈ alkylene. In some embodiments, L⁴ is independently -L⁷-NH—C(O)— or -L⁷-C(O)—NH—; and L⁷ is independently hydroxymethyl-substituted C₅-C₈ alkylene. In some embodiments, L⁴ is independently -L⁷-NH—C(O)— or -L⁷-C(O)—NH—; and L⁷ is independently unsubstituted C₅-C₈ alkylene.

In embodiments, the alkylene of L⁷ is unsubstituted or substituted with 1, 2 or 3 substituents selected from the group consisting of hydroxyl, oxo, carboxyl, mercapto, and amino.

In embodiments, the alkylene of L⁷ is unsubstituted or substituted with 1, 2 or 3 substituents selected from the group consisting of halogen, hydroxyl, carboxyl, oxo, and amino.

In some embodiments, L⁴ is independently oxo-substituted heteroalkylene, e.g., 2 to 20 membered, 2 to 12 membered, 2 to 10 membered, 2 to 8 membered, 2 to 6 membered, or 2 to 4 membered.

In some embodiments, L⁴ is independently

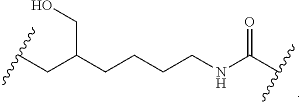 [SMILES: *CC(CO)CCCCNC(*)=O]

In embodiments, L⁴ is independently

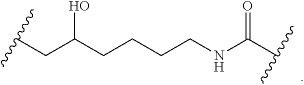 [SMILES: *CC(O)CCCCNC(*)=O]

In embodiments, L⁴ is independently

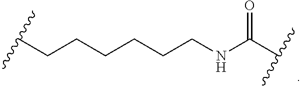 [SMILES: *CCCCCCNC(*)=O]

In embodiments, L⁴ is independently

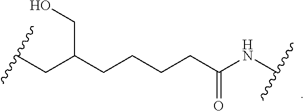 [SMILES: *CC(CO)CCCCC(=O)N*]

In embodiments, L⁴ is independently

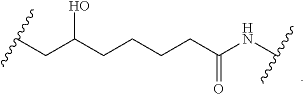 [SMILES: *CC(O)CCCCC(=O)N*]

In embodiments, L⁴ is independently

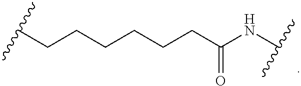 [SMILES: *CCCCCCC(=O)N*]

In embodiments, L⁴ is independently

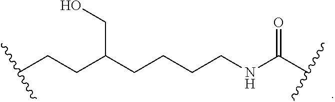 [SMILES: *CCC(CO)CCCCNC(*)=O]

In embodiments, L⁴ is independently

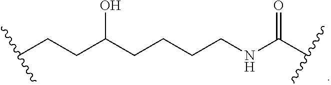 [SMILES: *CCC(O)CCCCNC(*)=O]

In embodiments, L⁴ is independently

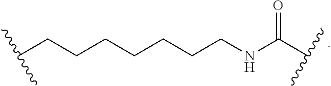 [SMILES: *CCCCCCCNC(*)=O]

In embodiments, L⁴ is independently

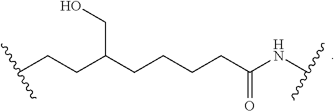 [SMILES: *CCC(CO)CCCCC(=O)N*]

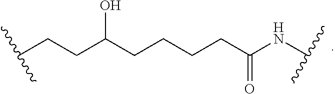 [SMILES: *CCC(O)CCCCC(=O)N*]

In embodiments, L⁴ is independently

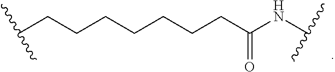 [SMILES: *CCCCCCCC(=O)N*]

In some embodiments, L³ is attached to a 3′ carbon of a 3′ terminal nucleotide of the sense strand of the double stranded nucleic acid.

In some embodiments, L³ is attached to a 5′ carbon of a 5′ terminal nucleotide of the sense strand of the double stranded nucleic acid.

In some embodiments, L³ is attached to a nucleobase of the double stranded oligonucleotide.

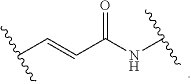 [SMILES: */C=C/C(=O)N*]

In some embodiments, L³ is independently H

In some embodiments, L³ is independently a bond.

In some embodiments, L³ is independently —OPO₂—O—.

In some embodiments, L³ is independently —OP(S)(OH)—O—.

In some embodiments, L³ is independently —O—.

In some embodiments, L⁴ is independently C₁-C₂₀ alkylene or 2 to 20 membered hetoeroalkylene, wherein the C₁-C₂₀ alkylene and 2 to 20 membered hetoeroalkylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁—C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is independently C₁-C₂₀ alkylene or 2 to 20 membered hetoeroalkylene, wherein the C₁-C₂₀ alkylene and 2 to 20 membered hetoeroalkylene are each independently unsubstituted or substituted with 1, 2, or 3, substituents independently selected from the group consisting of hydroxyl, oxo, carboxyl, amino, and halogen.

In some embodiments, L⁴ is independently -L⁷-NH—C(O)— or -L⁷-C(O)—NH—, wherein L⁷ is substituted or unsubstituted C₁-C₂₀ alkylene. In some embodiments, the C₁-C₂₀ alkylene of L⁷ is unsubstituted or substituted with 1, 2 or 3 substituents selected from the group consisting of halogen, hydroxyl, carboxyl, oxo, and amino.

In some embodiments, L⁴ is independently -L⁷-NH—C(O)— or -L⁷-C(O)—NH—, wherein L⁷ is selected from C₁-C₈ alkylene, C₃-C₈ alkylene, and C₅-C₈alkylene, wherein the C₁-C₈ alkylene, C₃-C₈ alkylene, and C₅-C₈alkylene are each independently unsubstituted or substituted with 1, 2 or 3 substituents selected from the group consisting of halogen, hydroxyl, carboxyl, oxo, and amino. In some embodiments, L⁴ is independently -L⁷-NH—C(O)—, wherein L⁷ is C₃-C₈ alkylene, wherein the C₃-C₈ alkylene is unsubstituted or substituted with 1, 2 or 3 substituents selected from the group consisting of hydroxyl, and oxo. In some embodiments, L⁴ is independently -L⁷-NH—C(O)—, wherein L⁷ is C₅-C₈alkylene, wherein the C₅-C₈alkylene is unsubstituted or substituted with 1, 2 or 3 substituents selected from the group consisting of hydroxyl, and oxo.

In some embodiments, L⁴ is independently

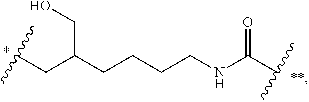 [SMILES: *CC(CO)CCCCNC(*)=O]

wherein * indicates the point of attachment to L³ and ** indicates the point of attachment to the fatty acid moiety.

In some embodiments, L⁴ is independently

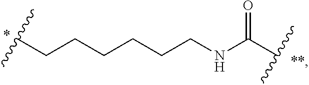 [SMILES: *CCCCCCNC(*)=O]

wherein * indicates the point of attachment to L³ and ** indicates the point of attachment to the fatty acid moiety.

In embodiments, -L³-L⁴- is independently -L⁷-NH—C(O)— or -L⁷-C(O)—NH—. In embodiments, L⁷ is independently substituted or unsubstituted heteroalkylene, e.g., 2 to 20 membered, 2 to 12 membered, 2 to 10 membered, 2 to 8 membered, 2 to 6 membered, or 2 to 4 membered. In embodiments, L⁷ is independently substituted heteroalkylene, e.g., 2 to 20 membered, 2 to 12 membered, 2 to 10 membered, 2 to 8 membered, 2 to 6 membered, or 2 to 4 membered. In embodiments, the heteroalkylene, e.g., 2 to 20 membered, 2 to 12 membered, 2 to 10 membered, 2 to 8 membered, 2 to 6 membered, or 2 to 4 membered heteroalkylene, is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, L⁷ is independently oxo-substituted heteroalkylene, e.g., 2 to 20 membered, 2 to 12 membered, 2 to 10 membered, 2 to 8 membered, 2 to 6 membered, or 2 to 4 membered. In embodiments, L⁷ is independently unsubstituted heteroalkylene, e.g., 2 to 20 membered, 2 to 12 membered, 2 to 10 membered, 2 to 8 membered, 2 to 6 membered, or 2 to 4 membered.

In embodiments, -L³-L⁴- is independently -L⁷-NH—C(O)—, wherein L⁷ is a 2 to 10 membered heteroalkylene, wherein the 2 to 10 membered heteroalkylene is unsubstituted or substituted with 1, 2, or 3 substituents selected from the group consisting of halogen, hydroxyl, carboxyl, oxo, and amino. In embodiments, -L³-L⁴- is independently -L⁷-NH—C(O)—, wherein L⁷ is a 2 to 10 membered heteroalkylene, wherein the 2 to 10 membered heteroalkylene is unsubstituted or substituted with 1, 2, or 3 substituents selected from the group consisting of hydroxyl, and oxo.

In embodiments, -L³-L⁴- is independently —O-L⁷-NH—C(O)— or —O-L⁷-C(O)—NH—. In embodiments, L⁷ is independently substituted or unsubstituted alkylene, e.g., C₁-C₂₀, C₁-C₁₂, C₁-C₈, C₁-C₆, C₁-C₄, or C₁-C₂. In embodiments, -L³-L⁴- is independently —O-L⁷-NH—C(O)— or —O-L⁷-C(O)—NH—; and L⁷ is independently substituted or unsubstituted alkylene, e.g., C₁-C₂₀, C₁-C₁₂, C₁-C₈, C₁-C₆, C₁-C₄, or C₁-C₂. In embodiments, -L³-L⁴- is independently —O-L⁷-NH—C(O)—; and L⁷ is independently substituted or unsubstituted alkylene, e.g., C₁-C₂₀, C₁-C₁₂, C₁-C₈, C₁-C₆, C₁-C₄, or C₁-C₂. In embodiments, the alkylene, e.g., C₁-C₂₀, C₁-C₁₂, C₁-C₈, C₁-C₆, C₁-C₄, or C₁-C₂, is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, -L³-L⁴- is independently-O-L⁷-C(O)—NH—; and L⁷ is independently substituted or unsubstituted C₁-C₈ alkylene. In embodiments, -L³-L⁴- is independently-O-L⁷-C(O)—NH—; and L⁷ is independently substituted C₁-C₈ alkylene. In embodiments, the C₁-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently-O-L⁷-C(O)—NH—; and L⁷ is independently hydroxy(OH)-substituted C₁-C₈ alkylene. In embodiments, -L³-L⁴- is independently-O-L⁷-C(O)—NH— and L⁷ is independently hydroxymethyl-substituted C₁-C₈ alkylene. In embodiments, -L³-L⁴- is independently-O-L⁷-C(O)—NH—; and L⁷ is independently unsubstituted C₁-C₈ alkylene.

In embodiments, -L³-L⁴- is independently-O-L⁷-C(O)—NH—; and L⁷ is independently substituted or unsubstituted C₃-C₈ alkylene. In embodiments, -L³-L⁴- is independently-O-L⁷-C(O)—NH—; and L⁷ is independently substituted C₃-C₈ alkylene. In embodiments, the C₃-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently-O-L⁷-C(O)—NH—; and L⁷ is independently hydroxy(OH)-substituted C₃-C₈ alkylene. In embodiments, -L³-L⁴- is independently-O-L⁷-C(O)—NH— and L⁷ is independently hydroxymethyl-substituted C₃-C₈ alkylene. In embodiments, -L³-L⁴- is independently-O-L⁷-C(O)—NH—; and L⁷ is independently unsubstituted C₃-C₈ alkylene.

In embodiments, -L³-L⁴- is independently —O-L⁷-C(O)—NH—; and L⁷ is independently substituted or unsubstituted C₅-C₈alkylene. In embodiments, -L³-L⁴- is independently-O-L⁷-C(O)—NH—; and L⁷ is independently substituted C₅-C₈alkylene. In embodiments, the C₅-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently-O-L⁷-C(O)—NH—; and L⁷ is independently hydroxy(OH)-substituted C₅-C₈alkylene. In embodiments, -L³-L⁴- is independently-O-L⁷-C(O)—NH— and L⁷ is independently hydroxymethyl-substituted C₅-C₈ alkylene. In embodiments, -L³-L⁴- is independently-O-L⁷-C(O)—NH—; and L⁷ is independently unsubstituted C₅-C₈alkylene.

In embodiments, -L³-L⁴- is independently —O-L⁷-NH—C(O)—; and L⁷ is independently substituted or unsubstituted C₁-C₈ alkylene. In embodiments, -L³-L⁴- is independently —O-L⁷-NH—C(O)—; and L⁷ is independently substituted C₁-C₈ alkylene. In embodiments, the C₁-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently —O-L⁷-NH—C(O)—; and L⁷ is independently hydroxy(OH)-substituted C₁-C₈ alkylene. In embodiments, -L³-L⁴- is independently —O-L⁷-NH—C(O)—; and L⁷ is independently hydroxymethyl-substituted C₁-C₈ alkylene. In embodiments, -L³-L⁴- is independently —O-L⁷-NH—C(O)—; and L⁷ is independently unsubstituted C₁-C₈ alkylene.

In embodiments, -L³-L⁴- is independently —O-L⁷-NH—C(O)—; and L⁷ is independently substituted or unsubstituted C₃-C₈ alkylene. In embodiments, -L³-L⁴- is independently —O-L⁷-NH—C(O)—; and L⁷ is independently substituted C₃-C₈ alkylene. In embodiments, the C₃-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently —O-L⁷-NH—C(O)—; and L⁷ is independently hydroxy(OH)-substituted C₃-C₈ alkylene. In embodiments, -L³-L⁴- is independently —O-L⁷-NH—C(O)—; and L⁷ is independently hydroxymethyl-substituted C₃-C₈ alkylene. In embodiments, -L³-L⁴- is independently —O-L⁷-NH—C(O)—; and L⁷ is independently unsubstituted C₃-C₈ alkylene.

In embodiments, -L³-L⁴- is independently —O-L⁷-NH—C(O)—; and L⁷ is independently substituted or unsubstituted C₅-C₈alkylene. In embodiments, -L³-L⁴- is independently —O-L⁷-NH—C(O)—; and L⁷ is independently substituted C₅-C₈alkylene. In embodiments, the C₅-C₈alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently —O-L⁷-NH—C(O)—; and L⁷ is independently hydroxy(OH)-substituted C₅-C₈alkylene. In embodiments, -L³-L⁴- is independently —O-L⁷-NH—C(O)—; and L⁷ is independently hydroxymethyl-substituted C₅-C₈ alkylene. In embodiments, -L³-L⁴- is independently —O-L⁷-NH—C(O)—; and L⁷ is independently unsubstituted C₅-C₈alkylene.

In embodiments, the alkylene of L⁷ is unsubstituted or substituted with 1, 2 or 3 substituents selected from the group consisting of halogen, hydroxyl, carboxyl, oxo, and amino.

In embodiments, -L³-L⁴- is independently —O-L⁷-NH—C(O)— or —O-L⁷-C(O)—NH—; and L⁷ is selected from C₁-C₈ alkylene, C₃-C₈ alkylene, and C₅-C₈alkylene, wherein the C₁-C₈ alkylene, C₃-C₈ alkylene, and C₅-C₈alkylene are each independently unsubstituted or substituted with 1, 2 or 3 substituents selected from the group consisting of hydroxyl, and oxo.

In embodiments, -L³-L⁴- is independently —O-L⁷-NH—C(O)—, wherein L⁷ is C₇ alkyl substituted with one hydroxyl group.

In embodiments, -L³-L⁴- is independently

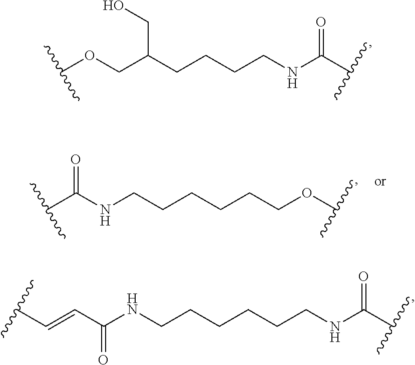 [SMILES: *OCC(CO)CCCCNC(*)=O ; *OCCCCCCNC(*)=O ; */C=C/C(=O)NCCCCCCNC(*)=O]

In embodiments, -L³-L⁴- is independently

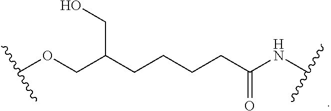 [SMILES: *NC(=O)CCCCC(CO)CO*]

In embodiments, -L³-L⁴- is independently

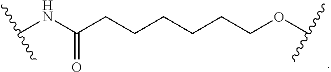 [SMILES: *NC(=O)CCCCCCO*]

In embodiments, -L³-L⁴- is independently

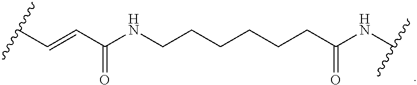 [SMILES: */C=C/C(=O)NCCCCCCC(=O)N*]

In embodiments, -L³-L⁴- is independently —OPO₂—O-L⁷-NH—C(O)— or —OPO₂—O-L⁷-C(O)—NH—. In embodiments, L⁷ is independently substituted or unsubstituted alkylene, e.g., C₁-C₂₀, C₁-C₁₂, C₁-C₈, C₁-C₆, C₁-C₄, or C₁-C₂. In embodiments, -L³-L⁴- is independently —OPO₂—O-L⁷-NH—C(O)— or —OPO₂—O-L⁷-C(O)—NH—; and L⁷ is independently substituted or unsubstituted alkylene, e.g., C₁-C₂₀, C₁-C₁₂, C₁-C₈, C₁-C₆, C₁-C₄, or C₁-C₂. In embodiments, -L³-L⁴- is independently —OPO₂—O-L⁷-NH—C(O)—; and L⁷ is independently substituted or unsubstituted alkylene, e.g., C₁-C₂₀, C₁-C₁₂, C₁-C₈, C₁-C₆, C₁-C₄, or C₁-C₂. In embodiments, -L³-L⁴- is independently —OPO₂—O-L⁷-C(O)—NH—; and L⁷ is independently substituted or unsubstituted alkylene, e.g., C₁-C₂₀, C₁-C₁₂, C₁-C₈, C₁-C₆, C₁-C₄, or C₁-C₂.

In embodiments, -L³-L⁴- is independently —OPO₂—O-L⁷-C(O)—NH—; and L⁷ is independently substituted or unsubstituted C₁-C₈ alkylene. In embodiments, -L³-L⁴- is independently —OPO₂—O-L⁷-C(O)—NH—; and L⁷ is independently substituted C₁-C₈ alkylene. In embodiments, the C₁-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently —OPO₂—O-L⁷-C(O)—NH—; and L⁷ is independently hydroxy(OH)-substituted C₁-C₈ alkylene. In embodiments, -L³-L⁴- is independently —OPO₂—O-L⁷-C(O)—NH—; and L⁷ is independently hydroxymethyl-substituted C₁-C₈ alkylene. In embodiments, -L³-L⁴- is independently —OPO₂—O-L⁷-C(O)—NH—; and L⁷ is independently unsubstituted C₁-C₈ alkylene.

In embodiments, -L³-L⁴- is independently —OPO₂—O-L⁷-C(O)—NH—; and L⁷ is independently substituted or unsubstituted C₃-C₈ alkylene. In embodiments, -L³-L⁴- is independently —OPO₂—O-L⁷-C(O)—NH—; and L⁷ is independently substituted C₃-C₈ alkylene. In embodiments, the C₃-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently —OPO₂—O-L⁷-C(O)—NH—; and L⁷ is independently hydroxy(OH)-substituted C₃-C₈ alkylene. In embodiments, -L³-L⁴- is independently —OPO₂—O-L⁷-C(O)—NH—; and L⁷ is independently hydroxymethyl-substituted C₃-C₈ alkylene. In embodiments, -L³-L⁴- is independently —OPO₂—O-L⁷-C(O)—NH—; and L⁷ is independently unsubstituted C₃-C₈ alkylene.

In embodiments, -L³-L⁴- is independently —OPO₂—O-L⁷-C(O)—NH—; and L⁷ is independently substituted or unsubstituted C₅-C₈alkylene. In embodiments, -L³-L⁴- is independently —OPO₂—O-L⁷-C(O)—NH—; and L⁷ is independently substituted C₅-C₈alkylene. In embodiments, the C₅-C₈alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently —OPO₂—O-L⁷-C(O)—NH—; and L⁷ is independently hydroxy(OH)-substituted C₅-C₈alkylene. In embodiments, -L³-L⁴- is independently —OPO₂—O-L⁷-C(O)—NH—; and L⁷ is independently hydroxymethyl-substituted C₅—C₈ alkylene. In embodiments, -L³-L⁴- is independently —OPO₂—O-L⁷-C(O)—NH—; and L⁷ is independently unsubstituted C₅-C₈alkylene.

In embodiments, -L³-L⁴- is independently —OPO₂—O-L⁷-NH—C(O)—; and L⁷ is independently substituted or unsubstituted C₁-C₈ alkylene. In embodiments, -L³-L⁴- is independently —OPO₂—O-L⁷-NH—C(O)—; and L⁷ is independently substituted C₁-C₈ alkylene. In embodiments, the C₁-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently —OPO₂—O-L⁷-NH—C(O)—; and L⁷ is independently hydroxy(OH)-substituted C₁-C₈ alkylene. In embodiments, -L³-L⁴- is independently —OPO₂—O-L⁷-NH—C(O)—; and L⁷ is independently hydroxymethyl-substituted C₁-C₈ alkylene. In embodiments, -L³-L⁴- is independently —OPO₂—O-L⁷-NH—C(O)—; and L⁷ is independently unsubstituted C₁-C₈ alkylene.

In embodiments, -L³-L⁴- is independently —OPO₂—O-L⁷-NH—C(O)—; and L⁷ is independently substituted or unsubstituted C₃-C₈ alkylene. In embodiments, -L³-L⁴- is independently —OPO₂—O-L⁷-NH—C(O)—; and L⁷ is independently substituted C₃-C₈ alkylene. In embodiments, the C₃-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently —OPO₂—O-L⁷-NH—C(O)—; and L⁷ is independently hydroxy(OH)-substituted C₃-C₈ alkylene. In embodiments, -L³-L⁴- is independently —OPO₂—O-L⁷-NH—C(O)—; and L⁷ is independently hydroxymethyl-substituted C₃-C₈ alkylene. In embodiments, -L³-L⁴- is independently —OPO₂—O-L⁷-NH—C(O)—; and L⁷ is independently unsubstituted C₃-C₈ alkylene.

In embodiments, -L³-L⁴- is independently —OPO₂—O-L⁷-NH—C(O)—; and L⁷ is independently substituted or unsubstituted C₅-C₈alkylene. In embodiments, -L³-L⁴- is independently —OPO₂—O-L⁷-NH—C(O)—; and L⁷ is independently substituted C₅-C₈alkylene. In embodiments, the C₅-C₈ alkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen. In embodiments, -L³-L⁴- is independently —OPO₂—O-L⁷-NH—C(O)—; and L⁷ is independently hydroxy(OH)-substituted C₅-C₈alkylene. In embodiments, -L³-L⁴- is independently —OPO₂—O-L⁷-NH—C(O)—; and L⁷ is independently hydroxymethyl-substituted C₅-C₈alkylene. In embodiments, -L³-L⁴- is independently —OPO₂—O-L⁷-NH—C(O)—; and L⁷ is independently unsubstituted C₅-C₈alkylene.

In embodiments, the alkylene of L⁷ is unsubstituted or substituted with 1, 2 or 3 substituents selected from the group consisting of halogen, hydroxyl, carboxyl, oxo, and amino.

In embodiments, -L³-L⁴- is independently —OPO₂—O-L⁷-NH—C(O)— or —OPO₂—O-L⁷-C(O)—NH—; and L⁷ is selected from C₁-C₈ alkylene, C₃-C₈ alkylene, and C₅-C₈ alkylene, wherein the C₁-C₈ alkylene, C₃-C₈ alkylene, and C₅-C₈alkylene are each independently unsubstituted or substituted with 1, 2 or 3 substituents selected from the group consisting of hydroxyl, and oxo.

In embodiments, -L³-L⁴- is independently —OPO₂—O-L⁷-NH—C(O)—, wherein L⁷ is C₇ alkyl substituted with one hydroxyl group.

In embodiments, -L³-L⁴- is independently

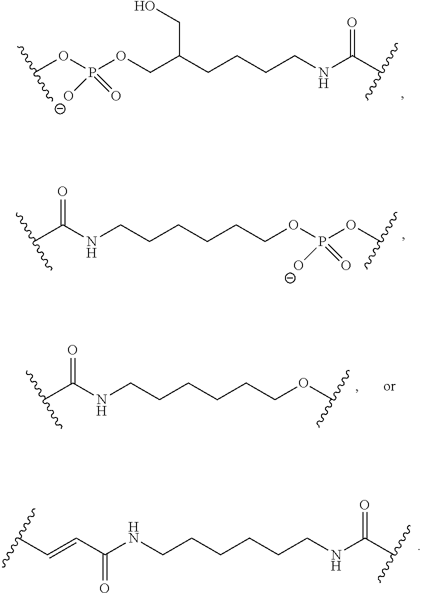 [SMILES: *OP(=O)([O-])OCC(CO)CCCCNC(*)=O ; *OP(=O)([O-])OCCCCCCNC(*)=O ; *OCCCCCCNC(*)=O ; */C=C/C(=O)NCCCCCCNC(*)=O ; "."]

In embodiments, -L³-L⁴- is independently

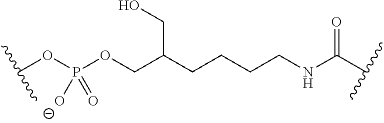 [SMILES: *OP(=O)([O-])OCC(CO)CCCCNC(*)=O]

and is attached to a 3′ carbon of the double-stranded oligonucleotide.

In embodiments, -L³-L⁴- is independently

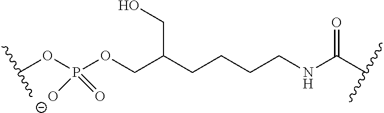 [SMILES: *OP(=O)([O-])OCC(CO)CCCCNC(*)=O]

and is attached to a 5′ carbon of the double-stranded oligonucleotide. In embodiments, -L³-L⁴- is independently

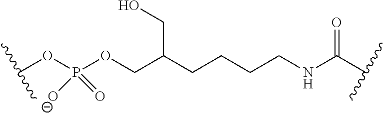 [SMILES: *OP(=O)([O-])OCC(CO)CCCCNC(*)=O]

and is attached to a 2′ carbon of the double-stranded oligonucleotide. In embodiments, -L³-L⁴- is independently

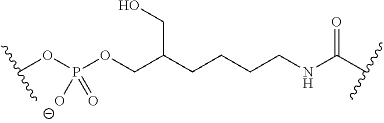 [SMILES: *OP(=O)([O-])OCC(CO)CCCCNC(*)=O]

and is attached to a nucleobase of the double-stranded oligonucleotide. In embodiments, -L³-L⁴- is independently

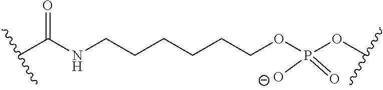 [SMILES: *OP(=O)([O-])OCCCCCCNC(*)=O]

and is attached to a 3′ carbon of the double-stranded oligonucleotide. In embodiments, -L³-L⁴- is independently

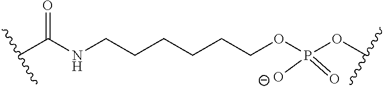 [SMILES: *OP(=O)([O-])OCCCCCCNC(*)=O]

and is attached to a 5′ carbon of the double-stranded oligonucleotide. In embodiments, -L³-L⁴- is independently

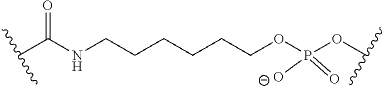 [SMILES: *OP(=O)([O-])OCCCCCCNC(*)=O]

and is attached to a 2′ carbon of the double-stranded oligonucleotide. In embodiments, -L³-L⁴- is independently

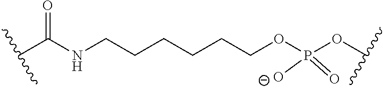 [SMILES: *OP(=O)([O-])OCCCCCCNC(*)=O]

and is attached to a nucleobase of the double-stranded oligonucleotide. In embodiments, -L³-L⁴- is independently

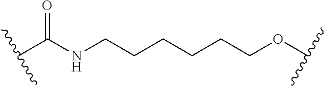 [SMILES: *OCCCCCCNC(*)=O]

and is attached to a 3′ carbon of the double-stranded oligonucleotide. In embodiments, -L³-L⁴- is independently

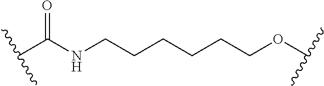 [SMILES: *OCCCCCCNC(*)=O]

and is attached to a 5′ carbon of the double-stranded oligonucleotide. In embodiments, -L³-L⁴- is independently

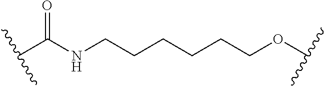 [SMILES: *OCCCCCCNC(*)=O]

and is attached to a 2′ carbon of the double-stranded oligonucleotide. In embodiments, -L³-L⁴- is independently

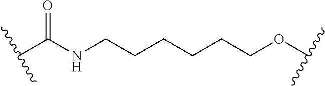 [SMILES: *OCCCCCCNC(*)=O]

and is attached to a nucleobase of the double-stranded oligonucleotide. In embodiments, -L³-L⁴- is independently

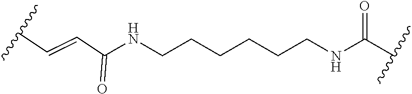 [SMILES: */C=C/C(=O)NCCCCCCNC(*)=O]

and is attached to a 3′ carbon of the double-stranded oligonucleotide. In embodiments, -L³-L⁴- is independently

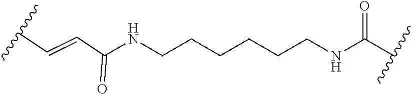 [SMILES: */C=C/C(=O)NCCCCCCNC(*)=O]

and is attached to a 5′ carbon of the double-stranded oligonucleotide. In embodiments, -L³-L⁴- is independently

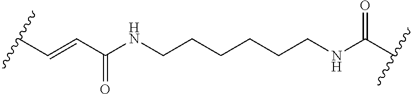 [SMILES: */C=C/C(=O)NCCCCCCNC(*)=O]

and is attached to a 2′ carbon of the double-stranded oligonucleotide. In embodiments, -L³-L⁴- is independently

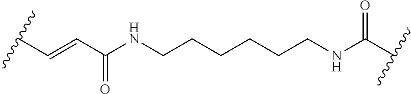 [SMILES: */C=C/C(=O)NCCCCCCNC(*)=O]

and is attached to a nucleobase of the double-stranded oligonucleotide.

In some embodiments, -L³-L⁴- is independently —O-L⁷-NH—C(O)—, wherein L⁷ is independently substituted or unsubstituted C₃-C₈ alkylene or C₅-C₈ alkylene. In some embodiments, L⁷ is C₃-C₈ alkylene or C₅-C₈ alkylene, wherein the C₃-C₈ alkylene or C₅-C₈ alkylene are each independently unsubstituted or substituted with 1, 2 or 3 substituents selected from the group consisting of halogen, hydroxyl, carboxyl, oxo, and amino. In some embodiments, L⁷ is C₃-C₈ alkylene or C₅-C₈ alkylene, wherein the C₃-C₈ alkylene or C₅-C₈ alkylene are each independently unsubstituted or substituted with 1, 2 or 3 substituents selected from the group consisting of hydroxyl, and oxo.

In some embodiments, -L³-L⁴- is independently

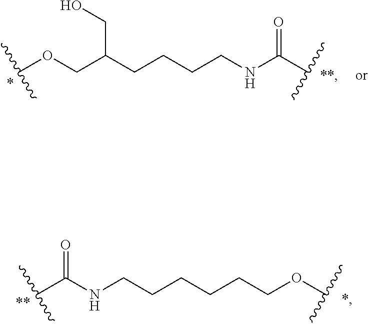 [SMILES: *OCC(CO)CCCCNC(*)=O ; *OCCCCCCNC(*)=O]

wherein * indicates the point of attachment to the double stranded nucleic acid and ** indicates the point of attachment to the fatty acid moiety.

In some embodiments, -L³-L⁴- is independently —OPO₂—O-L⁷-NH—C(O)—, wherein L⁷ is independently substituted or unsubstituted C₃-C₈ alkylene or C₅-C₈ alkylene. In some embodiments, L⁷ is C₃-C₈ alkylene or C₅-C₈ alkylene, wherein the C₃-C₈ alkylene or C₅-C₈ alkylene are each independently unsubstituted or substituted with 1, 2 or 3 substituents selected from the group consisting of halogen, hydroxyl, carboxyl, oxo, and amino.

In some embodiments, the alkylene of L⁷ is unsubstituted or substituted with 1, 2 or 3 substituents selected from the group consisting of halogen, hydroxyl, carboxyl, oxo, and amino.

In some embodiments, L⁷ is C₃-C₈ alkylene or C₅-C₈ alkylene, wherein the C₃-C₈ alkylene or C₅-C₈ alkylene are each independently unsubstituted or substituted with 1, 2 or 3 substituents selected from the group consisting of hydroxyl, and oxo.

In some embodiments, -L³-L⁴- is independently

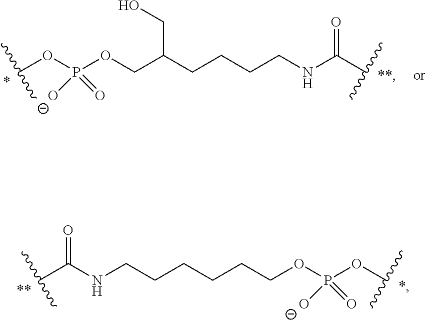 [SMILES: *OP(=O)([O-])OCC(CO)CCCCNC(*)=O ; *OP(=O)([O-])OCCCCCCNC(*)=O]

wherein * indicates the point of attachment to the double stranded nucleic acid and ** indicates the point of attachment to the fatty acid moiety.

In some embodiments, -L³-L⁴- is independently

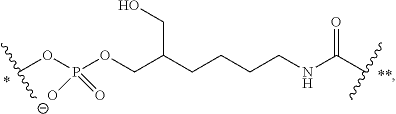 [SMILES: *OP(=O)([O-])OCC(CO)CCCCNC(*)=O]

wherein * indicates the point of attachment to a 3′ carbon of the sense strand of the double stranded nucleic acid and ** indicates the point of attachment to the fatty acid moiety.

In some embodiments, -L³-L⁴- is independently

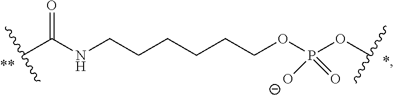 [SMILES: *OP(=O)([O-])OCCCCCCNC(*)=O]

wherein * indicates the point of attachment to a 5′ carbon of the sense strand of the double stranded nucleic acid and ** indicates the point of attachment to the fatty acid moiety.

In some embodiments, -L³-L⁴- is independently

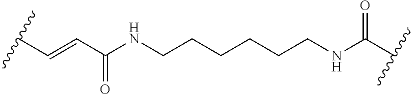 [SMILES: */C=C/C(=O)NCCCCCCNC(*)=O]

and is attached to a nucleotide base of the double stranded nucleic acid.

In embodiments of the linker, L³ and L⁴, a substituent for the alkylene, alkenylene, heteroalkylene, cycloalkylene, heterocycloalkylene, arylene or heteroarylene may be independently selected from oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, e.g., C₁-C₈ alkyl, C₁-C₆ alkyl, or C₁-C₄ alkyl, unsubstituted heteroalkyl, e.g., 2 to 8 membered heteroalkyl, 2 to 6 membered heteroalkyl, or 2 to 4 membered heteroalkyl, unsubstituted cycloalkyl, e.g., C₃-C₈ cycloalkyl, C₃-C₆ cycloalkyl, or C₅-C₆ cycloalkyl, unsubstituted heterocycloalkyl, e.g., 3 to 8 membered heterocycloalkyl, 3 to 6 membered heterocycloalkyl, or 5 to 6 membered heterocycloalkyl, unsubstituted aryl, e.g., C₆-C₁₀ aryl, C₁₀ aryl, or phenyl, unsubstituted heteroaryl, e.g., 5 to 10 membered heteroaryl, 5 to 9 membered heteroaryl, or 5 to 6 membered heteroaryl, unsubstituted alkoxy, e.g., C₁-C₈ alkoxy, C₁-C₆ alkoxy, or C₁-C₄ alkoxy, unsubstituted alkylthio, e.g., C₁-C₈ alkylthio, C₁-C₆ alkylthio, or C₁-C₄ alkylthio, and unsubstituted alkylamino, e.g., C₁-C₈ alkylamino, C₁-C₆ alkylamino, or C₁-C₄ alkylamino.

In embodiments of the linker, L³ and L⁴, a substituent for the alkylene, alkenylene, heteroalkylene, cycloalkylene, heterocycloalkylene, arylene or heteroarylene may be independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments of the linker, L³ and L⁴, a substituent for the alkylene, alkenylene, heteroalkylene, cycloalkylene, heterocycloalkylene, arylene or heteroarylene may be independently selected from the group consisting of halogen, hydroxyl, carboxyl, oxo, and amino.

In embodiments of the linker, L³ and L⁴, a substituent for the alkylene, alkenylene, heteroalkylene, cycloalkylene, heterocycloalkylene, arylene or heteroarylene may be independently selected from the group consisting of hydroxyl, and oxo.

In embodiments of L⁷, a substituent for the alkylene may be independently selected from oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, e.g., C₁-C₈ alkyl, C₁-C₆ alkyl, or C₁-C₄ alkyl, unsubstituted heteroalkyl, e.g., 2 to 8 membered heteroalkyl, 2 to 6 membered heteroalkyl, or 2 to 4 membered heteroalkyl, unsubstituted cycloalkyl, e.g., C₃-C₈ cycloalkyl, C₃-C₆ cycloalkyl, or C₅-C₆ cycloalkyl, unsubstituted heterocycloalkyl, e.g., 3 to 8 membered heterocycloalkyl, 3 to 6 membered heterocycloalkyl, or 5 to 6 membered heterocycloalkyl, unsubstituted aryl, e.g., C₆-C₁₀ aryl, C₁₀ aryl, or phenyl, unsubstituted heteroaryl, e.g., 5 to 10 membered heteroaryl, 5 to 9 membered heteroaryl, or 5 to 6 membered heteroaryl, unsubstituted alkoxy, e.g., C₁-C₈ alkoxy, C₁-C₆ alkoxy, or C₁-C₄ alkoxy, unsubstituted alkylthio, e.g., C₁-C₈ alkylthio, C₁-C₆ alkylthio, or C₁-C₄ alkylthio, and unsubstituted alkylamino, e.g., C₁-C₈ alkylamino, C₁-C₆ alkylamino, or C₁-C₄ alkylamino.

In embodiments of L⁷, a substituent for the alkylene may be independently selected from the group consisting of halogen, hydroxyl, carboxyl, oxo, and amino.

In embodiments of L⁷, a substituent for the alkylene may be independently selected from the group consisting of hydroxyl, and oxo.

### Fatty Acid Moiety

In some embodiments, the fatty acid moiety comprises a multibranched or non-linear structure. In some embodiments, the fatty acid moiety comprises 1 to 3 fatty acid chains. In some embodiments, the fatty acid moiety comprises 1 fatty acid chain. In some embodiments, the fatty acid moiety comprises 2 fatty acid chains. In some embodiments, the fatty acid moiety comprises 3 fatty acid chains. In some embodiments, the fatty acid moiety comprises 1 to 3 fatty acid chains each comprising an amide-containing alkyl group. In some embodiments, the fatty acid moiety comprises 1 fatty acid chain comprising an amide-containing alkyl group. In some embodiments, the fatty acid moiety comprises 2 fatty acid chains each comprising an amide-containing alkyl group. In some embodiments, the fatty acid moiety comprises 3 fatty acid chains each comprising an amide-containing alkyl group. In some embodiments, the fatty acid moiety comprises an alpha amino acid scaffold or a part thereof. In some embodiments, the alpha amino acid is selected from lysine, ornithine, alanine, arginine, asparagine, aspartic acid, cysteine, citrulline, glutamine, glutamic acid, glycine, histidine, isoleucine, leucine, methionine, phenylalanine, proline, serine, threonine, tryptophan, tyrosine, valine, diaminobutyric acid, and diaminopropionic acid. In some embodiments, the alpha amino acid is lysine or ornithine. In some embodiments, the alpha amino acid is L-lysine.

In some embodiments, the fatty acid moiety comprises a group of formula

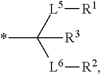

wherein
L⁵ is -L^{5A}-L^{5B}-L^{5C}-L^{5D}-L^{5E}-;
L⁶ is -L^{6A}-L^{6B}-L^{6C}-L^{6D}-L^{6E}-;
L^{5A}, L^{5B}, L^{5C}, L^{5D}, L^{5E}, L^{6A}, L^{6B} L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted C₁-C₂₀ alkylene, substituted or unsubstituted 2 to 20 membered heteroalkylene, substituted or unsubstituted C₃-C₁₀ cycloalkylene, substituted or unsubstituted 2 to 20 membered heterocycloalkylene, substituted or unsubstituted C₆-C₁₂ arylene, or substituted or unsubstituted 5 to 12 membered heteroarylene;
R¹ and R² are independently substituted or unsubstituted C₁-C₂₅ alkyl or C₁-C₂₅ alkenyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted C₁-C₂₀ alkyl, substituted or unsubstituted 2 to 20 membered heteroalkyl, substituted or unsubstituted C₃-C₁₀ cycloalkyl, substituted or unsubstituted 2 to 20 membered heterocycloalkyl, substituted or unsubstituted C₆-C₁₂ aryl, or substituted or unsubstituted 5 to 12 membered heteroaryl;
wherein * indicates the point of attachment to the rest of the molecule.

In embodiments, R³ is independently hydrogen. In embodiments, R³ is independently —NH₂. In embodiments, R³ is independently —OH. In embodiments, R³ is independently —SH. In embodiments, R³ is independently —C(O)H. In embodiments, R³ is independently —C(O)NH₂. In embodiments, R³ is independently —NHC(O)H. In embodiments, R³ is independently —NHC(O)OH. In embodiments, R³ is independently —NHC(O)NH₂. In embodiments, R³ is independently —C(O)OH. In embodiments, R³ is independently —OC(O)H. In embodiments, R³ is independently —N₃.

In embodiments, R³ is independently substituted or unsubstituted C₁-C₂₀ alkyl. In embodiments, R³ is independently substituted C₁-C₂₀ alkyl. In embodiments, R³ is independently unsubstituted C₁-C₂₀ alkyl. In embodiments, R³ is independently substituted or unsubstituted C₁-C₁₂ alkyl. In embodiments, R³ is independently substituted C₁-C₁₂ alkyl. In embodiments, R³ is independently unsubstituted C₁-C₁₂ alkyl. In embodiments, R³ is independently substituted or unsubstituted C₁-C₈ alkyl. In embodiments, R³ is independently substituted C₁-C₈ alkyl. In embodiments, R³ is independently unsubstituted C₁-C₈ alkyl. In embodiments, R³ is independently substituted or unsubstituted C₁-C₆ alkyl. In embodiments, R³ is independently substituted C₁-C₆ alkyl. In embodiments, R³ is independently unsubstituted C₁-C₆ alkyl. In embodiments, R³ is independently substituted or unsubstituted C₁-C₄ alkyl. In embodiments, R³ is independently substituted C₁-C₄ alkyl. In embodiments, R³ is independently unsubstituted C₁-C₄ alkyl. In embodiments, R³ is independently substituted or unsubstituted ethyl. In embodiments, R³ is independently substituted ethyl. In embodiments, R³ is independently unsubstituted ethyl. In embodiments, R³ is independently substituted or unsubstituted methyl. In embodiments, R³ is independently substituted methyl. In embodiments, R³ is independently unsubstituted methyl.

In embodiments, R³ is hydrogen.

In embodiments of R³, a substituent for the alkyl, heteroalkyl, cycloalkyl, heterocycloalkyl, aryl or heteroaryl may be independently selected from oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, e.g., C₁-C₈ alkyl, C₁-C₆ alkyl, or C₁-C₄ alkyl, unsubstituted heteroalkyl, e.g., 2 to 8 membered heteroalkyl, 2 to 6 membered heteroalkyl, or 2 to 4 membered heteroalkyl, unsubstituted cycloalkyl, e.g., C₃-C₈ cycloalkyl, C₃-C₆ cycloalkyl, or C₅-C₆ cycloalkyl, unsubstituted heterocycloalkyl, e.g., 3 to 8 membered heterocycloalkyl, 3 to 6 membered heterocycloalkyl, or 5 to 6 membered heterocycloalkyl, unsubstituted aryl, e.g., C₆-C₁₀ aryl, C₁₀ aryl, or phenyl, unsubstituted heteroaryl, e.g., 5 to 10 membered heteroaryl, 5 to 9 membered heteroaryl, or 5 to 6 membered heteroaryl, unsubstituted alkoxy, e.g., C₁-C₈ alkoxy, C₁-C₆ alkoxy, or C₁-C₄ alkoxy, unsubstituted alkylthio, e.g., C₁-C₈ alkylthio, C₁-C₆ alkylthio, or C₁-C₄ alkylthio, and unsubstituted alkylamino, e.g., C₁-C₈ alkylamino, C₁-C₆ alkylamino, or C₁-C₄ alkylamino.

In embodiments of R³, the alkyl, e.g., C₁-C₂₀ alkyl, C₁-C₁₂ alkyl, C₁-C₈ alkyl, C₁-C₆ alkyl, or C₁-C₄ alkyl, 2 to 20 membered heteroalkyl, C₃-C₁₀ cycloalkyl, 2 to 20 membered heterocycloalkyl, C₆-C₁₂ aryl, and 5 to 12 membered heteroaryl of R³ are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁶ is independently —NHC(O)—. In embodiments, L⁶ is independently —C(O)NH—. In embodiments, L⁶ is independently substituted or unsubstituted C₁-C₂₀ alkylene. In embodiments, L⁶ is independently substituted or unsubstituted 2 to 20 membered heteroalkylene. In embodiments, the C₁-C₂₀ alkylene and 2 to 20 membered heteroalkylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁶ is independently substituted or unsubstituted C₁-C₂₀ alkylene. In embodiments, L⁶ is independently substituted C₁-C₂₀ alkylene. In embodiments, L⁶ is independently unsubstituted C₁-C₂₀ alkylene. In embodiments, L⁶ is independently substituted or unsubstituted C₁-C₁₂ alkylene. In embodiments, L⁶ is independently substituted C₁-C₁₂ alkylene. In embodiments, L⁶ is independently unsubstituted C₁-C₁₂ alkylene. In embodiments, L⁶ is independently substituted or unsubstituted C₁-C₈ alkylene. In embodiments, L⁶ is independently substituted C₁-C₈ alkylene. In embodiments, L⁶ is independently unsubstituted C₁-C₈ alkylene. In embodiments, L⁶ is independently substituted or unsubstituted C₁-C₆ alkylene. In embodiments, L⁶ is independently substituted C₁-C₆ alkylene. In embodiments, L⁶ is independently unsubstituted C₁-C₆ alkylene. In embodiments, L⁶ is independently substituted or unsubstituted C₁-C₄ alkylene. In embodiments, L⁶ is independently substituted C₁-C₄ alkylene. In embodiments, L⁶ is independently unsubstituted C₁-C₄ alkylene. In embodiments, L⁶ is independently substituted or unsubstituted ethylene. In embodiments, L⁶ is independently substituted ethylene. In embodiments, L⁶ is independently unsubstituted ethylene. In embodiments, L⁶ is independently substituted or unsubstituted methylene. In embodiments, L⁶ is independently substituted methylene. In embodiments, L⁶ is independently unsubstituted methylene.

In embodiments, the alkylene of L⁶, e.g., C₁-C₂₀ alkylene, C₁-C₁₂ alkylene, C₁-C₈ alkylene, C₁-C₆ alkylene, or C₁-C₄ alkylene, is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁶ is independently C₁-C₂₀ alkylene, C₁-C₁₂ alkylene, or C₁-C₈ alkylene, wherein the C₁-C₂₀ alkylene, C₁-C₁₂ alkylene, or C₁-C₈ alkylene are each independently unsubstituted or substituted with 1, 2, or 3 substituents independently selected from the group consisting of hydroxyl, oxo, carboxyl, amino, and halogen.

In embodiments, L⁶ is independently substituted or unsubstituted 2 to 20 membered heteroalkylene. In embodiments, L⁶ is independently substituted 2 to 20 membered heteroalkylene. In embodiments, L⁶ is independently unsubstituted 2 to 20 membered heteroalkylene. In embodiments, L⁶ is independently substituted or unsubstituted 2 to 8 membered heteroalkylene. In embodiments, L⁶ is independently substituted 2 to 8 membered heteroalkylene. In embodiments, L⁶ is independently unsubstituted 2 to 8 membered heteroalkylene. In embodiments, L⁶ is independently substituted or unsubstituted 2 to 6 membered heteroalkylene. In embodiments, L⁶ is independently substituted 2 to 6 membered heteroalkylene. In embodiments, L⁶ is independently unsubstituted 2 to 6 membered heteroalkylene. In embodiments, L⁶ is independently substituted or unsubstituted 4 to 6 membered heteroalkylene. In embodiments, L⁶ is independently substituted 4 to 6 membered heteroalkylene. In embodiments, L⁶ is independently unsubstituted 4 to 6 membered heteroalkylene. In embodiments, L⁶ is independently substituted or unsubstituted 2 to 3 membered heteroalkylene. In embodiments, L⁶ is independently substituted 2 to 3 membered heteroalkylene. In embodiments, L⁶ is independently unsubstituted 2 to 3 membered heteroalkylene. In embodiments, L⁶ is independently substituted or unsubstituted 4 to 5 membered heteroalkylene. In embodiments, L⁶ is independently substituted 4 to 5 membered heteroalkylene. In embodiments, L⁶ is independently unsubstituted 4 to 5 membered heteroalkylene.

In embodiments, the heteroalkylene of L⁶, e.g., 2 to 20 membered, 2 to 8 membered, 2 to 6 membered, 4 to 6 membered, 2 to 3 membered, or 4 to 5 membered heteroalkylene, is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁶ is independently 2 to 20 membered heteroalkylene, 2 to 8 membered heteroalkylene, or 2 to 6 membered heteroalkylene, wherein the 2 to 20 membered heteroalkylene, 2 to 8 membered heteroalkylene, or 2 to 6 membered heteroalkylene are each independently unsubstituted or substituted with 1, 2, or 3 substituents independently selected from the group consisting of hydroxyl, oxo, carboxyl, amino, and halogen.

In embodiments, L⁶ is independently —NHC(O)—, —C(O)NH—, substituted or unsubstituted C₁-C₂₀ alkylene, or substituted or unsubstituted 2 to 20 membered heteroalkylene. In embodiments, the C₁-C₂₀ alkylene and 2 to 20 membered heteroalkylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁶ is independently —NHC(O)—, —C(O)NH—, substituted or unsubstituted C₁-C₁₂ alkylene, or substituted or unsubstituted 2 to 8 membered heteroalkylene. In embodiments, the C₁-C₁₂ alkylene and 2 to 8 membered heteroalkylene are each independently unsubstituted or substituted with 1, 2, or 3 substituents independently selected from the group consisting of hydroxyl, oxo, carboxyl, amino, and halogen.

In embodiments, L⁶ is-NHC(O)—.

In embodiments, L^{6A} is independently a bond or unsubstituted C₁-C₂₀ alkylene; L^{6B} is independently a bond, —NHC(O)—, or unsubstituted C₆-C₁₂ arylene; L^{6C} is independently a bond, unsubstituted C₁-C₂₀ alkylene, or unsubstituted C₆-C₁₂ arylene; L^{6D} is independently a bond or unsubstituted C₁-C₂₀ alkylene; and L⁶E is independently a bond or —NHC(O)—. In embodiments, L^{6A} is independently a bond or unsubstituted C₁-C₂₀ alkylene. In embodiments, L^{6B} is independently a bond, —NHC(O)—, or unsubstituted C₆-C₁₂ arylene. In embodiments, L^{6C} is independently a bond, unsubstituted C₁-C₂₀ alkylene, or unsubstituted C₆-C₁₂ arylene. In embodiments, L^{6D} is independently a bond or unsubstituted C₁-C₂₀ alkylene. In embodiments, L⁶E is independently a bond or —NHC(O)—.

In embodiments, L^{6A} is independently unsubstituted C₁-C₂₀ alkylene. In embodiments, L^{6A} is independently unsubstituted C₁-C₁₂ alkylene. In embodiments, L^{6A} is independently unsubstituted C₁-C₈ alkylene. In embodiments, L^{6A} is independently unsubstituted C₁-C₆ alkylene. In embodiments, L^{6A} is independently unsubstituted C₁-C₄ alkylene. In embodiments, L^{6A} is independently unsubstituted ethylene. In embodiments, L^{6A} is independently unsubstituted methylene. In embodiments, L^{6A} is independently a bond.

In embodiments, L^{6B} is independently a bond. In embodiments, L^{6B} is independently —NHC(O)—. In embodiments, L^{6B} is independently unsubstituted C₆-C₁₂ arylene. In embodiments, L^{6B} is independently unsubstituted C₆-C₁₀ arylene. In embodiments, L^{6B} is independently unsubstituted phenylene. In embodiments, L^{6B} is independently unsubstituted naphthylene.

In embodiments, L^{6C} is independently unsubstituted C₁-C₂₀ alkylene. In embodiments, L^{6C} is independently unsubstituted C₁-C₁₂ alkylene. In embodiments, L^{6C} is independently unsubstituted C₁-C₈ alkylene. L^{6C} is independently unsubstituted C₂-C₈ alkynylene. In embodiments, L^{6C} is independently unsubstituted C₁-C₆ alkylene. In embodiments, L^{6C} is independently unsubstituted C₁-C₄ alkylene. In embodiments, L^{6C} is independently unsubstituted ethylene. In embodiments, L^{6C} is independently unsubstituted methylene. In embodiments, L^{6C} is independently a bond or unsubstituted alkynylene (e.g., C₂-C₂₀, C₂-C₁₂, C₂-C₈, C₂-C₆, C₂-C₄, or C₂-C₂). In embodiments, L^{6C} is independently unsubstituted C₂-C₂₀ alkynylene. In embodiments, L^{6C} is independently unsubstituted C₂-C₁₂ alkynylene. In embodiments, L^{6C} is independently unsubstituted C₂-C₅ alkynylene. In embodiments, L^{6C} is independently unsubstituted C₂-C₆ alkynylene. In embodiments, L^{6C} is independently unsubstituted C₂-C₄ alkynylene. In embodiments, L^{6C} is independently unsubstituted ethynylene. In embodiments, L^{6C} is independently unsubstituted arylene (e.g., C₆-C₁₂, C₆-C₁₀, or phenyl). In embodiments, L^{6C} is independently unsubstituted C₆-C₁₂ arylene. In embodiments, L^{6C} is independently unsubstituted C₆-C₁₀ arylene. In embodiments, L^{6C} is independently unsubstituted phenylene. In embodiments, L^{6C} is independently unsubstituted naphthylene. In embodiments, L^{6C} is independently a bond.

In embodiments, L^{6D} is independently unsubstituted C₁-C₂₀ alkylene. In embodiments, L^{6D} is independently unsubstituted C₁-C₁₂ alkylene. In embodiments, L^{6A} is independently unsubstituted C₁-C₈ alkylene. In embodiments, L^{6D} is independently unsubstituted C₁-C₆ alkylene. In embodiments, L^{6D} is independently unsubstituted C₁-C₄ alkylene. In embodiments, L^{6D} is independently unsubstituted ethylene. In embodiments, L^{6D} is independently unsubstituted methylene. In embodiments, L^{6D} is independently a bond.

In embodiments, L⁶E is independently a bond. In embodiments, L⁶E is independently —NHC(O)—.

In embodiments, L^{6A} is independently a bond or unsubstituted C₁-C₈ alkylene. In embodiments, L^{6B} is independently a bond, —NHC(O)—, or unsubstituted phenylene. In embodiments, L^{6C} is independently a bond, unsubstituted C₂-C₅ alkynylene, or unsubstituted phenylene. In embodiments, L^{6D} is independently a bond or unsubstituted C₁-C₈ alkylene. In embodiments, L⁶E is independently a bond or —NHC(O)—.

In embodiments of L⁶, L^{6A}, L^{6B} L^{6C}, L^{6D}, and L^{6E}, a substituent for the alkylene, heteroalkylene, cycloalkylene, heterocycloalkylene, arylene or heteroarylene may be independently selected from oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, e.g., C₁-C₈ alkyl, C₁-C₆ alkyl, or C₁-C₄ alkyl, unsubstituted heteroalkyl, e.g., 2 to 8 membered heteroalkyl, 2 to 6 membered heteroalkyl, or 2 to 4 membered heteroalkyl, unsubstituted cycloalkyl, e.g., C₃-C₈ cycloalkyl, C₃-C₆ cycloalkyl, or C₅-C₆ cycloalkyl, unsubstituted heterocycloalkyl, e.g., 3 to 8 membered heterocycloalkyl, 3 to 6 membered heterocycloalkyl, or 5 to 6 membered heterocycloalkyl, unsubstituted aryl, e.g., C₆-C₁₀ aryl, C₁₀ aryl, or phenyl, unsubstituted heteroaryl, e.g., 5 to 10 membered heteroaryl, 5 to 9 membered heteroaryl, or 5 to 6 membered heteroaryl, unsubstituted alkoxy, e.g., C₁-C₈ alkoxy, C₁-C₆ alkoxy, or C₁-C₄ alkoxy, unsubstituted alkylthio, e.g., C₁-C₈ alkylthio, C₁-C₆ alkylthio, or C₁-C₄ alkylthio, and unsubstituted alkylamino, e.g., C₁-C₈ alkylamino, C₁-C₆ alkylamino, or C₁-C₄ alkylamino.

In embodiments of L⁶, L^{6A}, L^{6B}, L^{6C}, L^{6D}, and L^{6E}, the alkylene, e.g., C₁-C₂₀ alkylene, C₁-C₁₂ alkylene, C₁-C₈ alkylene, C₁-C₆ alkylene, C₁-C₄ alkylene, heteroalkylene, e.g., 2 to 20 membered, 2 to 8 membered, 2 to 6 membered, 4 to 6 membered, 2 to 3 membered, 4 to 5 membered heteroalkylene, cycloalkylene, heterocycloalkylene, arylene and heteroarylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁶ is independently a bond,

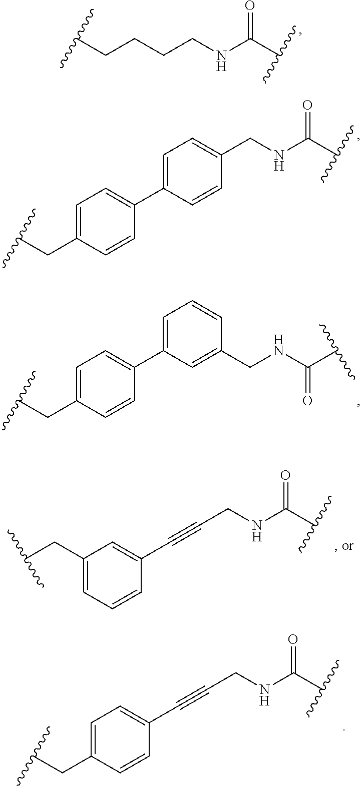 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L⁶ is independently a bond. In embodiments L⁶ is independently

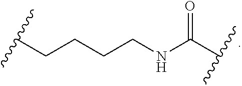 [SMILES: *CCCCNC(*)=O]

In embodiments, L⁶ is independently

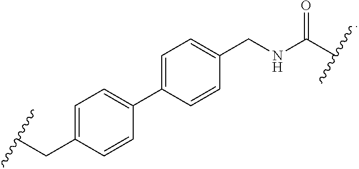 [SMILES: *Cc1ccc(-c2ccc(CNC(*)=O)cc2)cc1]

In embodiments, L⁶ is independently

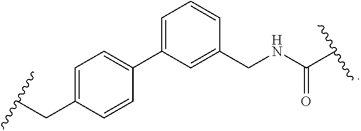 [SMILES: *Cc1ccc(-c2cccc(CNC(*)=O)c2)cc1]

In embodiments, L⁶ is independently

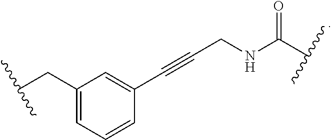 [SMILES: *Cc1cccc(C#CCNC(*)=O)c1]

In embodiments, L⁶ is independently

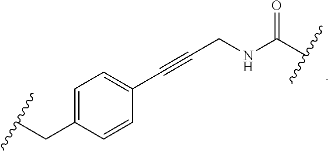 [SMILES: *Cc1ccc(C#CCNC(*)=O)cc1]

In embodiments, L⁶ is —NHC(O)—.

In embodiments, L⁵ is independently —NHC(O)—. In embodiments, L⁵ is independently —C(O)NH—. In embodiments, L⁵ is independently substituted or unsubstituted C₁-C₂₀ alkylene. In embodiments, L⁵ is independently substituted or unsubstituted 2 to 20 membered heteroalkylene. In embodiments, the C₁-C₂₀ alkylene and 2 to 20 membered heteroalkylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁵ is independently substituted or unsubstituted C₁-C₂₀ alkylene. In embodiments, L⁵ is independently substituted C₁-C₂₀ alkylene. In embodiments, L⁵ is independently unsubstituted C₁-C₂₀ alkylene. In embodiments, L⁵ is independently substituted or unsubstituted C₁-C₁₂ alkylene. In embodiments, L⁵ is independently substituted C₁-C₁₂ alkylene. In embodiments, L⁵ is independently unsubstituted C₁-C₁₂ alkylene. In embodiments, L⁵ is independently substituted or unsubstituted C₁-C₈ alkylene. In embodiments, L⁵ is independently substituted C₁-C₈ alkylene. In embodiments, L⁵ is independently unsubstituted C₁-C₈ alkylene. In embodiments, L⁵ is independently substituted or unsubstituted C₁-C₆ alkylene. In embodiments, L⁵ is independently substituted C₁-C₆ alkylene. In embodiments, L⁵ is independently unsubstituted C₁-C₆ alkylene. In embodiments, L⁵ is independently substituted or unsubstituted C₁-C₄ alkylene. In embodiments, L⁵ is independently substituted C₁-C₄ alkylene. In embodiments, L⁵ is independently unsubstituted C₁-C₄ alkylene. In embodiments, L⁵ is independently substituted or unsubstituted ethylene. In embodiments, L⁵ is independently substituted ethylene. In embodiments, L⁵ is independently unsubstituted ethylene. In embodiments, L⁵ is independently substituted or unsubstituted methylene. In embodiments, L⁵ is independently substituted methylene. In embodiments, L⁵ is independently unsubstituted methylene. In embodiments, the alkylene, e.g., C₁-C₂₀ alkylene, C₁-C₁₂ alkylene, C₁-C₈ alkylene, C₁-C₆ alkylene, or C₁-C₄ alkylene, when substituted, is substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈alkylamino, C₁-C₆ alkylamino, or C₁-C₄ alkylamino, and halogen.

In embodiments, L⁵ is independently C₁-C₂₀ alkylene, C₁-C₁₂ alkylene, or C₁-C₈ alkylene, wherein the C₁-C₂₀ alkylene, C₁-C₁₂ alkylene, or C₁-C₈ alkylene are each independently unsubstituted or substituted with 1, 2, or 3 substituents independently selected from the group consisting of hydroxyl, oxo, carboxyl, amino, and halogen.

In embodiments, L⁵ is independently substituted or unsubstituted 2 to 20 membered heteroalkylene. In embodiments, L⁵ is independently substituted 2 to 20 membered heteroalkylene. In embodiments, L⁵ is independently unsubstituted 2 to 20 membered heteroalkylene. In embodiments, L⁵ is independently substituted or unsubstituted 2 to 8 membered heteroalkylene. In embodiments, L⁵ is independently substituted 2 to 8 membered heteroalkylene. In embodiments, L⁵ is independently unsubstituted 2 to 8 membered heteroalkylene. In embodiments, L⁵ is independently substituted or unsubstituted 2 to 6 membered heteroalkylene. In embodiments, L⁵ is independently substituted 2 to 6 membered heteroalkylene. In embodiments, L⁵ is independently unsubstituted 2 to 6 membered heteroalkylene. In embodiments, L⁵ is independently substituted or unsubstituted 4 to 6 membered heteroalkylene. In embodiments, L⁵ is independently substituted 4 to 6 membered heteroalkylene. In embodiments, L⁵ is independently unsubstituted 4 to 6 membered heteroalkylene. In embodiments, L⁵ is independently substituted or unsubstituted 2 to 3 membered heteroalkylene. In embodiments, L⁵ is independently substituted 2 to 3 membered heteroalkylene. In embodiments, L⁵ is independently unsubstituted 2 to 3 membered heteroalkylene. In embodiments, L⁵ is independently substituted or unsubstituted 4 to 5 membered heteroalkylene. In embodiments, L⁶ is independently substituted 4 to 5 membered heteroalkylene. In embodiments, L⁶ is independently unsubstituted 4 to 5 membered heteroalkylene. In embodiments, the heteroalkylene, e.g., 2 to 20 membered, 2 to 8 membered, 2 to 6 membered, 4 to 6 membered, 2 to 3 membered, or 4 to 5 membered heteroalkylene, when substituted, is substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁵ is independently 2 to 20 membered heteroalkylene, 2 to 8 membered heteroalkylene, or 2 to 6 membered heteroalkylene, wherein the 2 to 20 membered heteroalkylene, 2 to 8 membered heteroalkylene, or 2 to 6 membered heteroalkylene are each independently unsubstituted or substituted with 1, 2, or 3 substituents independently selected from the group consisting of hydroxyl, oxo, carboxyl, amino, and halogen.

In embodiments, L⁵ is independently —NHC(O)—, —C(O)NH—, substituted or unsubstituted C₁-C₂₀ alkylene, or substituted or unsubstituted 2 to 20 membered heteroalkylene. In embodiments, the C₁-C₂₀ alkylene and 2 to 20 membered heteroalkylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁵ is independently —NHC(O)—, —C(O)NH—, substituted or unsubstituted C₁-C₁₂ alkylene, or substituted or unsubstituted 2 to 8 membered heteroalkylene. In embodiments, the C₁-C₁₂ alkylene and 2 to 8 membered heteroalkylene are each independently unsubstituted or substituted with 1, 2, or 3 substituents independently selected from the group consisting of hydroxyl, oxo, carboxyl, amino, and halogen.

In embodiments, L⁵ is independently —NHC(O)—.

In embodiments, L^{5A} is independently a bond or unsubstituted C₁-C₂₀ alkylene; L^{5B} is independently a bond, —NHC(O)—, or unsubstituted C₆-C₁₂ arylene; L⁵C is independently a bond, unsubstituted C₁-C₂₀ alkylene, or unsubstituted C₆-C₁₂ arylene; L^{5D} is independently a bond or unsubstituted C₁-C₂₀ alkylene; and L^{5E} is independently a bond or —NHC(O)—. In embodiments, L^{5A} is independently a bond or unsubstituted C₁-C₂₀ alkylene. In embodiments, L^{5B} is independently a bond, —NHC(O)—, or unsubstituted C₆-C₁₂ arylene. In embodiments, L⁵C is independently a bond, unsubstituted C₁-C₂₀ alkylene, or unsubstituted C₆-C₁₂ arylene. In embodiments, L^{5D} is independently a bond or unsubstituted C₁-C₂₀ alkylene. In embodiments, L^{5E} is independently a bond or —NHC(O)—.

In embodiments, L^{5A} is independently unsubstituted C₁-C₂₀ alkylene. In embodiments, L^{5A} is independently unsubstituted C₁-C₁₂ alkylene. In embodiments, L^{5A} is independently unsubstituted C₁-C₈ alkylene. In embodiments, L^{5A} is independently unsubstituted C₁-C₆ alkylene. In embodiments, L^{5A} is independently unsubstituted C₁-C₄ alkylene. In embodiments, L^{5A} is independently unsubstituted ethylene. In embodiments, L^{5A} is independently unsubstituted methylene. In embodiments, L^{5A} is independently a bond.

In embodiments, L^{5B} is independently a bond. In embodiments, L^{5B} is independently —NHC(O)—. In embodiments, L^{5B} is independently unsubstituted C₆-C₁₂ arylene. In embodiments, L^{5B} is independently unsubstituted C₆-C₁₀ arylene. In embodiments, L^{5B} is independently unsubstituted phenylene. In embodiments, L^{5B} is independently unsubstituted naphthylene.

In embodiments, L^{5C} is independently unsubstituted C₁-C₂₀ alkylene. In embodiments, L^{5C} is independently unsubstituted C₁-C₁₂ alkylene. In embodiments, L^{5C} is independently unsubstituted C₁-C₈ alkylene. L^{5C} is independently unsubstituted C₂-C₈ alkynylene. In embodiments, L^{5C} is independently unsubstituted C₁-C₆ alkylene. In embodiments, L^{5C} is independently unsubstituted C₁-C₄ alkylene. In embodiments, L^{5C} is independently unsubstituted ethylene. In embodiments, L^{5C} is independently unsubstituted methylene. In embodiments, L^{5C} is independently unsubstituted C₂-C₂₀ alkynylene. In embodiments, L^{5C} is independently unsubstituted C₂-C₁₂ alkynylene. In embodiments, L^{5C} is independently unsubstituted C₂-C₅ alkynylene. In embodiments, L^{5C} is independently unsubstituted C₂-C₆ alkynylene. In embodiments, L^{5C} is independently unsubstituted C₂-C₄ alkynylene. In embodiments, L^{5C} is independently unsubstituted ethynylene. In embodiments, L^{5C} is independently unsubstituted C₆-C₁₂ arylene. In embodiments, L^{5C} is independently unsubstituted C₆-C₁₀ arylene. In embodiments, L^{5C} is independently unsubstituted phenylene. In embodiments, L^{5C} is independently unsubstituted naphthylene. In embodiments, L^{5C} is independently a bond.

In embodiments, L^{5D} is independently unsubstituted C₁-C₂₀ alkylene. In embodiments, L^{5D} is independently unsubstituted C₁-C₁₂ alkylene. In embodiments, L^{5D} is independently unsubstituted C₁-C₈ alkylene. In embodiments, L^{5D} is independently unsubstituted C₁-C₆ alkylene. In embodiments, L^{5D} is independently unsubstituted C₁-C₄ alkylene. In embodiments, L^{5D} is independently unsubstituted ethylene. In embodiments, L^{5D} is independently unsubstituted methylene. In embodiments, L^{5D} is independently a bond.

In embodiments, L^{5E} is independently a bond. In embodiments, L^{5E} is independently —NHC(O)—.

In embodiments, L^{5A} is independently a bond or unsubstituted C₁-C₈ alkylene. In embodiments, L^{5B} is independently a bond, —NHC(O)—, or unsubstituted phenylene. In embodiments, L^{5C} is independently a bond, unsubstituted C₂-C₅ alkynylene, or unsubstituted phenylene. In embodiments, L^{5D} is independently a bond or unsubstituted C₁-C₈ alkylene. In embodiments, L^{5E} is independently a bond or —NHC(O)—.

In embodiments of L⁵, L^{5A}, L^{5B} L^{5C}, L^{5D}, and L^{5E}, a substituent for the alkylene, heteroalkylene, cycloalkylene, heterocycloalkylene, arylene or heteroarylene may be independently selected from oxo, halogen, —CF₃, —CCl₃, —CBr₃, —Cl₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCI₃, —OCHF₂, —OCHCl₂, —OCHBr₂, —OCHI₂, —OCH₂F, —OCH₂Cl, —OCH₂Br, —OCH₂I, unsubstituted alkyl, e.g., C₁-C₈ alkyl, C₁-C₆ alkyl, or C₁-C₄ alkyl, unsubstituted heteroalkyl, e.g., 2 to 8 membered heteroalkyl, 2 to 6 membered heteroalkyl, or 2 to 4 membered heteroalkyl, unsubstituted cycloalkyl, e.g., C₃-C₈ cycloalkyl, C₃-C₆ cycloalkyl, or C₅-C₆ cycloalkyl, unsubstituted heterocycloalkyl, e.g., 3 to 8 membered heterocycloalkyl, 3 to 6 membered heterocycloalkyl, or 5 to 6 membered heterocycloalkyl, unsubstituted aryl, e.g., C₆-C₁₀ aryl, C₁₀ aryl, or phenyl, unsubstituted heteroaryl, e.g., 5 to 10 membered heteroaryl, 5 to 9 membered heteroaryl, or 5 to 6 membered heteroaryl, unsubstituted alkoxy, e.g., C₁-C₈ alkoxy, C₁-C₆ alkoxy, or C₁-C₄ alkoxy, unsubstituted alkylthio, e.g., C₁-C₈ alkylthio, C₁-C₆ alkylthio, or C₁-C₄ alkylthio, and unsubstituted alkylamino, e.g., C₁-C₈ alkylamino, C₁-C₆ alkylamino, or C₁-C₄ alkylamino.

In embodiments of L⁵, L^{5A}, L^{5B} L^{5C}, L^{5D}, and L^{5E}, the alkylene, e.g., C₁-C₂₀ alkylene, C₁-C₁₂ alkylene, C₁-C₈ alkylene, C₁-C₆ alkylene, C₁-C₄ alkylene, heteroalkylene, e.g., 2 to 20 membered, 2 to 8 membered, 2 to 6 membered, 4 to 6 membered, 2 to 3 membered, 4 to 5 membered heteroalkylene, cycloalkylene, heterocycloalkylene, arylene and heteroarylene are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, L⁵ is independently a bond

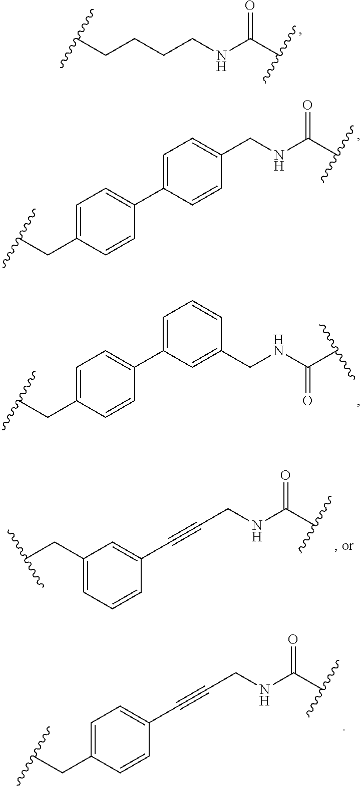 [SMILES: *CCCCNC(*)=O ; *Cc1ccc(-c2ccc(CNC(*)=O)cc2)cc1 ; *Cc1ccc(-c2cccc(CNC(*)=O)c2)cc1 ; *Cc1cccc(C#CCNC(*)=O)c1 ; *Cc1ccc(C#CCNC(*)=O)cc1]

In embodiments, L⁵ is independently a bond. In embodiments, L⁵ is independently

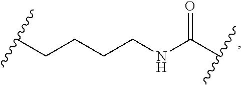 [SMILES: *CCCCNC(*)=O]

In embodiments, L⁵ is independently

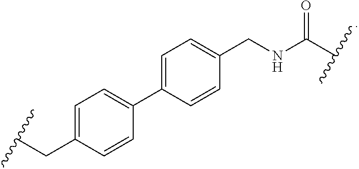 [SMILES: *Cc1ccc(-c2ccc(CNC(*)=O)cc2)cc1]

In embodiments, L⁵ is independently

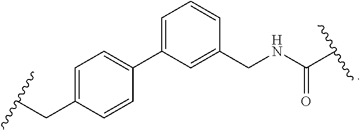 [SMILES: *Cc1ccc(-c2cccc(CNC(*)=O)c2)cc1]

In embodiments, L⁵ is independently

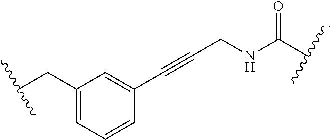 [SMILES: *Cc1cccc(C#CCNC(*)=O)c1]

In embodiments, L⁵ is independently

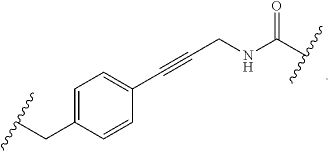 [SMILES: *Cc1ccc(C#CCNC(*)=O)cc1]

In embodiments, R¹ and R² are each independently substituted or unsubstituted C₁-C₂₅ alkyl or C₂-C₂₅ alkenyl. In embodiments, the C₁-C₂₅ alkyl and C₂-C₂₅ alkenyl are each independently unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In embodiments, R¹ and R² are each independently substituted or unsubstituted C₁-C₂₅ alkyl or C₂-C₂₅ alkenyl, wherein the C₁-C₂₅ alkyl and C₂-C₂₅ alkenyl, when substituted, are each independently substituted with 1, 2, or 3 substituents independently selected from the group consisting of hydroxyl, oxo, carboxyl, amino, and halogen.

In embodiments, R¹ and R² are each independently unsubstituted C₁-C₂₅ alkyl or C₂-C₂₅ alkenyl.

In embodiments, R¹ and R² are each independently unsubstituted C₁-C₂₅ alkyl.

In embodiments, R¹ is unsubstituted alkyl, e.g., C₁-C₂₅, C₁-C₂₀, C₁-C₁₇, C₁-C₁₂, C₁-C₈, C₁-C₆, C₁-C₄, or C₁-C₂. In embodiments, R¹ is unsubstituted unbranched alkyl, e.g., C₁-C₂₅, C₁-C₂₀, C₁-C₁₇, C₁-C₁₂, C₁-C₈, C₁-C₆, C₁-C₄, or C₁-C₂.

In embodiments, R¹ is unsubstituted alkenyl, e.g., C₂-C₂₅, C₂-C₂₀, C₂-C₁₇, C₂-C₁₂, C₂-C₈, C₂-C₆, or C₂-C₄. In embodiments, R¹ is unsubstituted unbranched alkenyl, e.g., C₂-C₂₅, C₂-C₂₀, C₂-C₁₇, C₂-C₁₂, C₂-C₈, C₂-C₆, or C₂-C₄.

In embodiments, R¹ is unsubstituted C₁-C₁₉ alkyl. In embodiments, R¹ is unsubstituted C₉-C₁₉ alkyl. In embodiments, R¹ is unsubstituted C₁₁-C₁₉ alkyl. In embodiments, R¹ is unsubstituted C₁₃-C₁₉ alkyl. In embodiments, R¹ is unsubstituted unbranched C₁-C₁₉ alkyl. In embodiments, R¹ is unsubstituted unbranched C₉-C₁₉ alkyl. In embodiments, R¹ is unsubstituted unbranched C₁₁-C₁₉ alkyl. In embodiments, R¹ is unsubstituted unbranched C₁₃-C₁₉ alkyl.

In embodiments, R¹ is unsubstituted C₁-C₁₇ alkyl. In embodiments, R¹ is unsubstituted C₁₁-C₁₇ alkyl. In embodiments, R¹ is unsubstituted C₁₂-C₁₆ alkyl. In embodiments, R¹ is unsubstituted C₁₃ alkyl. In embodiments, R¹ is unsubstituted unbranched C₁-C₁₇ alkyl. In embodiments, R¹ is unsubstituted unbranched C₁₁-C₁₇ alkyl. In embodiments, R¹ is unsubstituted unbranched C₁₂-C₁₆ alkyl. In embodiments, R¹ is unsubstituted unbranched C₁₃ alkyl. In embodiments, R¹ is unsubstituted unbranched C₁₄ alkyl. In embodiments, R¹ is unsubstituted unbranched C₁₅ alkyl. In embodiments, R¹ is unsubstituted unbranched C₁₆ alkyl.

In embodiments, R² is unsubstituted alkyl, e.g., C₁-C₂₅, C₁-C₂₀, C₁-C₁₇, C₁-C₁₂, C₁-C₈, C₁-C₆, C₁-C₄, or C₁-C₂. In embodiments, R² is unsubstituted unbranched alkyl, e.g., C₁-C₂₅, C₁-C₂₀, C₁-C₁₇, C₁-C₁₂, C₁-C₈, C₁-C₆, C₁-C₄, or C₁-C₂.

In embodiments, R² is unsubstituted alkenyl, e.g., C₂-C₂₅, C₂-C₂₀, C₂-C₁₇, C₂-C₁₂, C₂-C₈, C₂-C₆, or C₂-C₄. In embodiments, R² is unsubstituted unbranched alkenyl, e.g., C₂-C₂₅, C₂-C₂₀, C₂-C₁₇, C₂-C₁₂, C₂-C₈, C₂-C₆, or C₂-C₄.

In embodiments, R² is unsubstituted C₁-C₁₉ alkyl. In embodiments, R² is unsubstituted C₉-C₁₉ alkyl. In embodiments, R² is unsubstituted C₁₁-C₁₉ alkyl. In embodiments, R² is unsubstituted C₁₃-C₁₉ alkyl. In embodiments, R² is unsubstituted unbranched C₁-C₁₉ alkyl. In embodiments, R² is unsubstituted unbranched C₉-C₁₉ alkyl. In embodiments, R² is unsubstituted unbranched C₁₁-C₁₉ alkyl. In embodiments, R² is unsubstituted unbranched C₁₃-C₁₉ alkyl.

In embodiments, R² is unsubstituted C₁-C₁₇ alkyl. In embodiments, R² is unsubstituted C₁₁-C₁₇ alkyl. In embodiments, R² is unsubstituted C₁₂-C₁₆ alkyl. In embodiments, R² is unsubstituted C₁₃ alkyl. In embodiments, R² is unsubstituted unbranched C₁-C₁₇ alkyl. In embodiments, R² is unsubstituted unbranched C₁₁-C₁₇ alkyl. In embodiments, R² is unsubstituted unbranched C₁₂-C₁₆ alkyl. In embodiments, R² is unsubstituted unbranched C₁₃ alkyl. In embodiments, R² is unsubstituted unbranched C₁₄ alkyl. In embodiments, R² is unsubstituted unbranched C₁₅ alkyl. In embodiments, R² is unsubstituted unbranched C₁₆ alkyl.

In embodiments, at least one of R¹ and R² is unsubstituted C₁-C₁₉ alkyl. In embodiments, at least one of R¹ and R² is unsubstituted C₉-C₁₉ alkyl. In embodiments, at least one of R¹ and R² is unsubstituted C₁₁-C₁₉ alkyl. In embodiments, at least one of R¹ and R² is unsubstituted C₁₃-C₁₉ alkyl.

In some embodiments, the fatty acid moiety is a group of formula

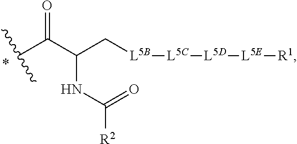

wherein L^{5B} L^{5C}, L^{5D}, L^{5E}, R¹ and R² are defined according to embodiments disclosed herein; and * indicates the point of attachment to the linker.

In some embodiments, the fatty acid moiety is a group of formula

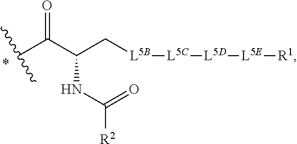

wherein L^{5B} L^{5C}, L^{5D}, L^{5E}, R¹ and R² are defined according to embodiments disclosed herein; and * indicates the point of attachment to the linker.

In some embodiments, the fatty acid moiety is a group of formula

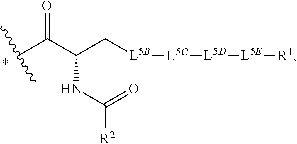

wherein L^{5B} L^{5C}, L^{5D}, L^{5E}, R¹ and R² are defined according to embodiments disclosed herein; and * indicates the point of attachment to the linker.

In some embodiments, the fatty acid moiety is a group of formula

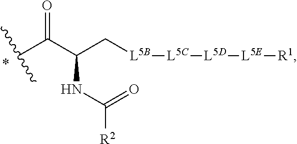

wherein L^{5B} L^{5C}, L^{5D}, L^{5E}, R¹ and R² are defined according to embodiments disclosed herein; and * indicates the point of attachment to the linker.

In some embodiments, the fatty acid moiety is a group of formula

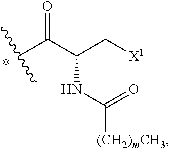

wherein
X¹ is

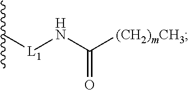

L¹ is —(CH₂)ₙ—, —(CH₂)ₙL²(CH₂)ₙ—, or a bond; L² is —C(═O)NH—, —C(═O)O—, —OC(═O)O—, —NHC(═O)O—, —NHC(═O)NH—, —C(═S)NH—, —C(═O)S—, —NH—, O (oxygen), or S (sulfur), and wherein each m is independently an integer from 10 to 18 and wherein each n is independently an integer from 1 to 6; and
* indicates the point of attachment to the linker.

### Conjugates

In some embodiments, the disclosure provides a dsRNAi agent, or a pharmaceutically acceptable salt thereof, of formula (I)

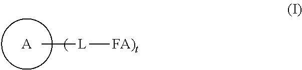

wherein:
A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
L is a linker;
FA is a fatty acid moiety capable of facilitating delivery of the double stranded nucleic acid into a cell, e.g., skeletal muscle cell; and
t is an integer from 1 to 5.

In some embodiments, a dsRNAi agent provided herein includes a compound having the structure of formula (II):

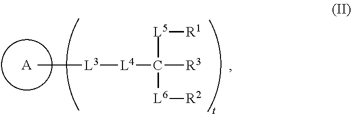

or a pharmaceutically acceptable salt thereof,
wherein
A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
L³ and L⁴ are independently a bond, —NH—, —O—, —S—, —C(O)—, —NHC(O)—, —NHC(O)NH—, —C(O)O—, —OC(O)—, —C(O)NH—, —OPO₂—O—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
L⁵ is -L^{5A}-L^{5B}-L^{5C}-L^{5D}-L^{5E}-;
L⁶ is -L^{6A}-L^{6B}-L^{6C}-L^{6D}-L^{6E}-;
L^{5A}, L^{5B} L^{5C}, L^{5D} L^{5E} L^{6A}, L^{6B} L^{6C}, L^{6D}, and L^{6E} are independently a bond, —NH—, —O—, —S—, —C(O)—, —NHC(O)—, —NHC(O)NH—, —C(O)O—, —OC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene;
R¹ and R² are independently unsubstituted C₁-C₂₅ alkyl or unsubstituted C₁-C₂₅ alkenyl;
R³ is hydrogen, —NH₂, —OH, —SH, —C(O)H, —C(O)NH₂, —NHC(O)H, —NHC(O)OH, —NHC(O)NH₂, —C(O)OH, —OC(O)H, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
t is an integer from 1 to 5.

In some embodiments, A is a double stranded nucleic acid comprising a sense strand and an antisense strand forming a duplex with the sense strand, wherein each of the antisense strand and the sense strand is independently 17 to 25 nucleotides in length.

In some embodiments, A is a double stranded nucleic acid comprising a sense strand and an antisense strand forming a duplex with the sense strand, wherein each of the antisense strand and the sense strand is independently 17 to 25 nucleotides in length, the nucleotide sequence of the antisense strand is at least 90% complementary to SEQ ID NO: 1, the nucleotide sequence of the sense strand having no more than two mismatches to the nucleotide sequence of the antisense strand in the duplex.

In some embodiments, at least one of R¹ and R² is unsubstituted C₉-C₁₉ alkyl.

Further embodiments of A, L³, L⁴, L⁵, L⁶, R¹, R² and R³ are defined hereinbefore and throughout the disclosure.

In some embodiments, a compound provided herein includes a compound having the structure of formula (II), or a pharmaceutically acceptable salt thereof,

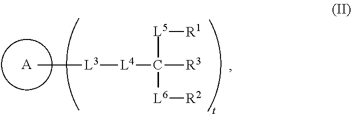

wherein:
A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
or
A is a double stranded nucleic acid comprising a sense strand and an antisense strand, the antisense strand forming a duplex with the sense strand, wherein each of the antisense strand and the sense strand is independently 17 to 25 nucleotides in length, wherein the nucleotide sequence of the antisense strand is at least 90% complementary to SEQ ID NO: 1, and the nucleotide sequence of the sense strand has no more than two mismatches to the nucleotide sequence of the antisense strand;
L³ is independently
   (i) a bond;

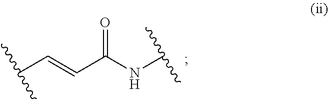 [SMILES: */C=C/C(=O)N*]

(iii) —OP(O)(OH)O—;
(iv) —OP(S)(OH)O—; or
(v) —O—;
L⁴ is independently:
   (i) substituted or unsubstituted 2 to 20 membered heteroalkylene; or
   (ii) -L⁷-NH—C(O)— or -L⁷-C(O)—NH—, wherein L⁷ is substituted or unsubstituted C₁-C₂₀ alkylene; or

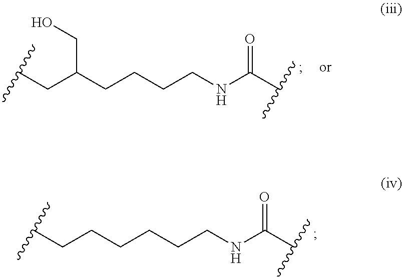 [SMILES: *CC(CO)CCCCNC(*)=O ; *CCCCCCNC(*)=O]

L⁵ is substituted or unsubstituted 2 to 20 membered heteroalkylene, or -L^{5A}L^{5B}L^{5C}L^{5D}L^{5E}-, wherein L^{5A} is independently unsubstituted C₁-C₈ alkylene; L^{5B} is independently NHC(O); L^{5C} is independently a bond; L^{5D} is independently a bond or unsubstituted C₁-C₈ alkylene; and L^{5E} is independently a bond or NHC(O);
L⁶ is —NHC(O)—;
R¹ and R² are independently unsubstituted C₁₁-C₁₉ alkyl or unsubstituted C₁₁-C₁₉ alkenyl;
R³ is hydrogen; and
t is 1.

In some embodiments, L³ is a bond. In some embodiments, L³ is

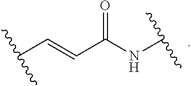 [SMILES: */C=C/C(=O)N*]

In some embodiments, L³ is —OP(O)(OH)O—. In some embodiments, L³ is —O—

In some embodiments, L⁴ is a substituted or unsubstituted 2 to 20 membered heteroalkylene. In some embodiments, the 2 to 20 membered heteroalkylene is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is a 2 to 20 membered heteroalkylene, wherein the 2 to 20 membered heteroalkylene is unsubstituted or substituted with 1, 2, or 3, substituents independently selected from the group consisting of hydroxyl, oxo, carboxyl, amino, and halogen.

In some embodiments, L⁴ is -L⁷-NH—C(O)—, wherein L⁷ is unsubstituted or substituted C₁-C₈ alkylene, e.g., C₃-C₈ alkylene, C₅-C₈alkylene. In some embodiments, the C₁-C₈ alkylene, e.g., C₃-C₈ alkylene, C₅-C₈alkylene, is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁴ is -L⁷-NH—C(O)—, wherein L⁷ is unsubstituted or substituted C₁-C₈ alkylene, C₃-C₈ alkylene, or C₅-C₈alkylene. In some embodiments, the C₁-C₈ alkylene, C₃-C₈ alkylene, or C₅-C₈alkylene, is each independently unsubstituted or substituted with 1, 2, or 3, substituents independently selected from the group consisting of hydroxyl, oxo, carboxyl, amino, and halogen.

In some embodiments, L⁴ is -L⁷-NH—C(O)—, wherein L⁷ is unsubstituted or substituted C₃-C₈ alkylene, wherein the C₃-C₈ alkylene, when substituted, is substituted with 1, 2, or 3 substituents independently selected from the group consisting of hydroxyl, and oxo.

In some embodiments, L⁴ is -L⁷-NH—C(O)—, wherein L⁷ is unsubstituted or substituted C₅-C₈alkylene, wherein the C₅-C₈ alkylene, when substituted, is substituted with 1, 2, or 3 substituents independently selected from the group consisting of hydroxyl, and oxo.

In some embodiments, L⁴ is -L⁷-NH—C(O)— and L⁷ is methylene or ethylene. In some embodiments, L⁴ is -L⁷-C(O)—NH— and L⁷ is methylene or ethylene. In some embodiments, L⁴ is -L⁷-NH—C(O)— and L⁷ is hydroxyl substituted C₇ alkyl. In some embodiments, L⁴ is -L⁷-NH—C(O)— and L⁷ is unsubstituted C₆ alkyl. In some embodiments, L⁴ is

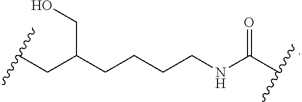 [SMILES: *CC(CO)CCCCNC(*)=O]

In some embodiments, L⁴ is

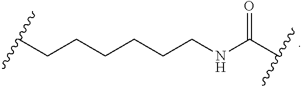 [SMILES: *CCCCCCNC(*)=O]

In some embodiments, -L³-L⁴- is

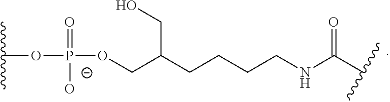 [SMILES: *OP(=O)([O-])OCC(CO)CCCCNC(*)=O]

In some embodiments, -L³-L⁴- is

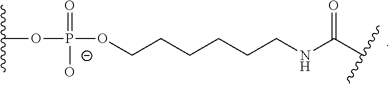 [SMILES: *OP(=O)([O-])OCCCCCCNC(*)=O]

In some embodiments, -L³-L⁴- is attached to the 3′ end (e.g., 3′ carbon) of the sense strand. In some embodiments, -L³-L⁴- is attached to the 5′ end (e.g., 5′ carbon) of the sense strand. In some embodiments, -L³-L⁴- is attached to the terminal 3′ end (e.g., terminal 3′ carbon) of the sense strand. In some embodiments, -L³-L⁴- is attached to the terminal 5′ end (e.g., terminal 5′ carbon) of the sense strand.

In some embodiments, L⁵ is substituted or unsubstituted 2 to 20 membered heteroalkylene, e.g., 2 to 8 membered heteroalkylene, 2 to 6 membered heteroalkylene, or -L^{5A}L^{5B}L^{5C}L^{5D}L^{5E}-. In some embodiments, the 2 to 20 membered heteroalkylene, e.g., 2 to 8 membered heteroalkylene, 2 to 6 membered heteroalkylene, is unsubstituted or substituted with 1, 2, 3, 4 or 5 substituents independently selected from the group consisting of hydroxyl, oxo, carboxyl, unsubstituted alkyl, e.g., C₁-C₈ alkyl, C₁-C₆ alkyl, or C₁-C₄ alkyl, unsubstituted alkoxy, e.g., C₁-C₈ alkoxy, C₁-C₆ alkoxy, or C₁-C₄ alkoxy, mercapto, unsubstituted alkylthio, e.g., C₁-C₈ alkylthio, C₁-C₆ alkylthio, or C₁-C₄ alkylthio, amino, unsubstituted alkylamino, e.g., C₁-C₈ alkylamino, C₁-C₆ alkylamino, or C₁-C₄ alkylamino, and halogen.

In some embodiments, L⁵ is a 2 to 20 membered heteroalkylene, wherein the 2 to 20 membered heteroalkylene is unsubstituted or substituted with 1, 2, or 3 substituents selected from hydroxyl, oxo, carboxyl, amino, and halogen.

In some embodiments, L⁵ is a 2 to 8 membered heteroalkylene, wherein the 2 to 8 membered heteroalkylene is unsubstituted or substituted with 1, 2, or 3 substituents selected from hydroxyl, oxo, carboxyl, amino, and halogen.

In some embodiments, L⁵ is a 2 to 6 membered heteroalkylene, wherein the 2 to 6 membered heteroalkylene is unsubstituted or substituted with 1, 2, or 3 substituents selected from hydroxyl, oxo, carboxyl, amino, and halogen.

In some embodiments, L^{5A} is independently unsubstituted C₂-C₅ alkylene. In some embodiments, L^{5A} is independently unsubstituted C₃-C₈ alkylene. In some embodiments, L^{5A} is independently unsubstituted C₄-C₈ alkylene. In some embodiments, L^{5A} is independently unsubstituted C₃ alkylene. In some embodiments, L^{5A} is independently unsubstituted C₄ alkylene. In some embodiments, L^{5A} is independently unsubstituted C₅ alkylene. In some embodiments, L^{5A} is independently unsubstituted C₆ alkylene. In some embodiments, L^{5A} is independently unsubstituted C₇ alkylene. In some embodiments, L^{5A} is independently unsubstituted C₃ alkylene. In some embodiments, L^{5D} is a bond. In some embodiments, L^{5E} is a bond. In some embodiments, L^{5D} and L^{5E} are bonds.

In some embodiments, L⁵ is

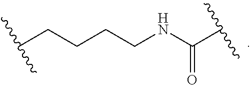 [SMILES: *CCCCNC(*)=O]

In some embodiments, L⁵ is

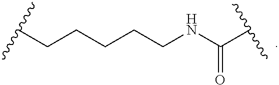 [SMILES: *CCCCCNC(*)=O]

In some embodiments, L⁵ is

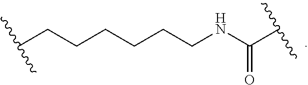 [SMILES: *CCCCCCNC(*)=O]

In some embodiments, R¹ is unsubstituted C₁₁-C₁₅ alkyl. In some embodiments, R¹ is unsubstituted C₁-C₁₃ alkyl. In some embodiments, R¹ is unsubstituted C₁₁ alkyl. In some embodiments, R¹ is unsubstituted C₁₂ alkyl. In some embodiments, R¹ is unsubstituted C₁₃ alkyl. In some embodiments, R¹ is unsubstituted C₁₄ alkyl. In some embodiments, R¹ is unsubstituted C₁₅ alkyl.

In some embodiments, R² is unsubstituted C₁-C₁₅ alkyl. In some embodiments, R² is unsubstituted C₁-C₁₃ alkyl. In some embodiments, R² is unsubstituted C₁₁ alkyl. In some embodiments, R² is unsubstituted C₁₂ alkyl. In some embodiments, R² is unsubstituted C₁₃ alkyl. In some embodiments, R² is unsubstituted C₁₄ alkyl. In some embodiments, R² is unsubstituted C₁₅ alkyl.

In some embodiments, R¹ and R² are each independently unsubstituted C₁-C₁₅ alkyl. In some embodiments, R¹ and R² are each independently unsubstituted C₁-C₁₃ alkyl. In some embodiments, R¹ is unsubstituted C₁₁ alkyl and R² is unsubstituted C₁₁ alkyl. In some embodiments, R¹ is unsubstituted C₁₂ alkyl and R² is unsubstituted C₁₂ alkyl. In some embodiments, R¹ is unsubstituted C₁₃ alkyl and R² is unsubstituted C₁₃ alkyl. In some embodiments, R¹ is unsubstituted C₁₄ alkyl and R² is unsubstituted C₁₄ alkyl. In some embodiments, R¹ is unsubstituted C₁₅ alkyl and R² is unsubstituted C₁₅ alkyl.

In some embodiments, provided herein is a compound, or a pharmaceutically acceptable salt thereof, of formula (Ia)

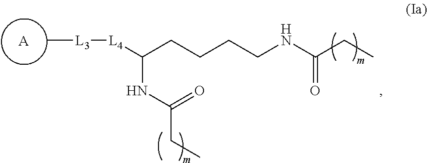

wherein:
   each m, at each occurrence, is independently an integer from 10 to 18;
-L³-L⁴- is defined according to any one of embodiments disclosed herein;
and A is defined according to any one of embodiments disclosed herein.

In some embodiments, the compound of formula (Ia) has formula (Ib)

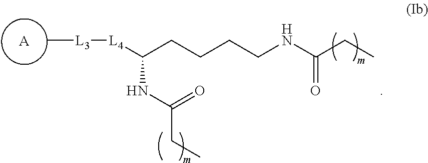

In some embodiments, the compound of formula (Ia) has formula (Ic)

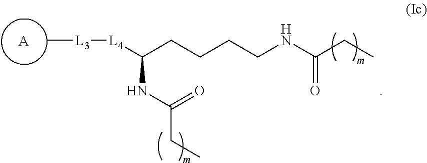

In some embodiments, the L³ is attached to the 3′ terminal end of the sense strand. some embodiments, the L³ is attached to the 5′ terminal end of the sense strand. In some embodiments, the L³ is attached to the 3′ terminal end of the antisense strand. In some embodiments, the L³ is attached to the 5′ terminal end of the antisense strand.

In some embodiments, the L³ is attached to the 3′ terminal end of the sense strand.

In some embodiments, L³ is attached to the 3′ terminal end of the sense strand, wherein the sense strand comprises at least 17 contiguous nucleotides selected from the nucleotide sequence of any one of SEQ ID Nos: 3 to 293. In some embodiments, L³ is attached to the 3′ terminal end of the sense strand, wherein the sense strand comprises at least 18 contiguous nucleotides selected from the nucleotide sequence of any one of SEQ ID Nos: 3 to 293. In some embodiments, L³ is attached to the 3′ terminal end of the sense strand, wherein the sense strand comprises at least 19 contiguous nucleotides selected from the nucleotide sequence of any one of SEQ ID Nos: 3 to 293.

In some embodiments, provided herein is a compound, or a pharmaceutically acceptable salt thereof, of formula (Id)

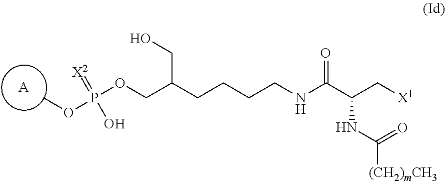

wherein
A is a modified double stranded nucleic acid having suppressing activity of DMPK gene expression, comprising a sense strand and an antisense strand forming a duplex with the sense strand, wherein each of the antisense strand and the sense strand is independently 10 to 30 nucleotides in length,
wherein the modified double stranded nucleic acid is conjugated at the terminal 3′carbon of the sense strand of the modified double stranded nucleic acid;
X² is selected from O and S;

X¹ is

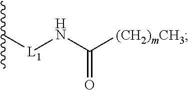

L¹ is —(CH₂)ₙ—, —(CH₂)ₙL²(CH₂)ₙ—, or a bond;
L² is —C(═O)NH—, —C(═O)O—, —OC(═O)O—, —NHC(═O)O—, —NHC(═O)NH—, —C(═S)NH—, —C(═O)S—, —NH—, O (oxygen), or S (sulfur), and wherein each m is independently an integer from 10 to 18 and wherein each n is independently an integer from 1 to 6.

Further embodiments of A are disclosed hereinbefore and throughout the disclosure.

In embodiments, X₁ is:

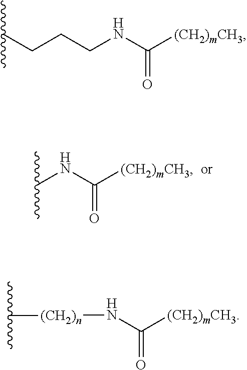

In embodiments, X₁ is

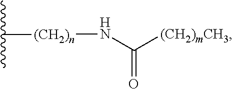

each m is 10, and n is 3. In embodiments, X₁ is

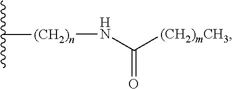

each m is 11, and n is 3. In embodiments, X₁ is

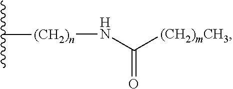

each m is 12, and n is 3. In embodiments, X₁ is

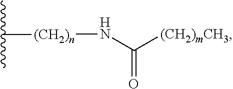

each m is 13, and n is 3. In embodiments, X₁ is

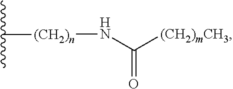

each m is 14, and n is 3. In embodiments, X₁ is

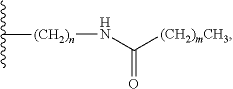

each m is 15, and n is 3. In embodiments, X₁ is

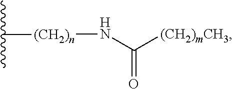

each m is 16, and n is 3. In embodiments, X₁ is

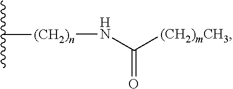

each m is 17, and n is 3. In embodiments, X₁ is

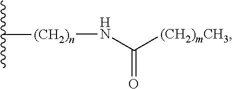

each m is 18, and n is 3. In embodiments, X₁ is

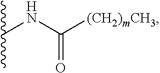

each m is 10. In embodiments, X₁ is

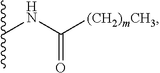

and each m is 11. In embodiments, X₁ is

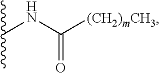

and each m is 12. In embodiments, X₁ is

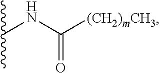

and each m is 13. In embodiments, X₁ is

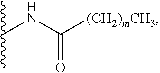

and each m is 14. In embodiments, X₁ is

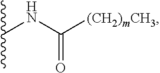

and each m is 15. In embodiments, X₁ is

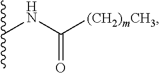

and each m is 16. In embodiments, X₁ is

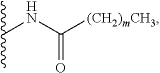

and each m is 17. In embodiments, X₁ is

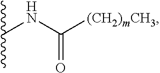

and each m is 18.

In embodiments, X₁ is

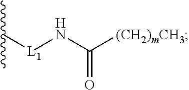

L₁ is —(CH₂)₃C(═O)NH(CH₂)₅—; and each m is 10. In embodiments, X₁ is

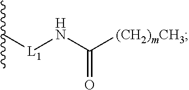

L₁ is —(CH₂)₃C(═O)NH(CH₂)₅—; and each m is 11. In embodiments, X₁ is

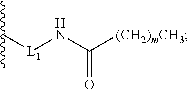

L₁ is —(CH₂)₃C(═O)NH(CH₂)₅—; and each m is 12. In embodiments, X₁ is

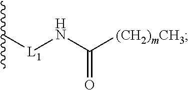

L₁ is —(CH₂)₃C(═O)NH(CH₂)₅—; and each m is 13. In embodiments, X₁ is

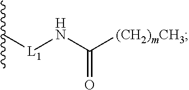

L₁ is —(CH₂)₃C(═O)NH(CH₂)₅—; and each m is 14. In embodiments, X₁ is

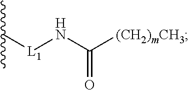

L₁ is —(CH₂)₃C(═O)NH(CH₂)(—; and each m is 15. In embodiments, X₁ is

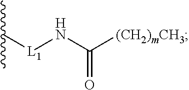

L₁ is —(CH₂)₃C(═O)NH(CH₂)₅—; and each m is 16. In embodiments, X₁ is

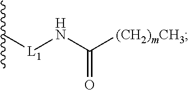

L₁ is —(CH₂)₃C(═O)NH(CH₂)₅—; and each m is 17 In embodiments, X₁ is

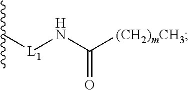

L₁ is —(CH₂)₃C(═O)NH(CH₂)₅—; and each m is 18.

In embodiments, L₁ is a bond; and each m is independently an integer from 10 to 16. In embodiments, L₁ is a bond; and each m is independently an integer from 12 to 16. In embodiments, L₁ is a bond; and each m is independently an integer from 12 to 14. In embodiments, L₁ is a bond; and each m is 14. In embodiments, L₁ is —(CH₂)ₙL²(CH₂)ₙ—; L² is —C(═O)NH—; each m is independently an integer from 10 to 16; and each n is independently an integer from 1 to 6. In embodiments, L₁ is —(CH₂)ₙL²(CH₂)ₙ—; L² is —C(═O)NH—; each m is independently an integer from 12 to 16; and each n is independently an integer from 1 to 6. In embodiments, L₁ is —(CH₂)ₙL²(CH₂)ₙ—; L² is —C(═O)NH—; each m is independently an integer from 12 to 14; and each n is independently an integer from 1 to 6. In embodiments, L₁ is —(CH₂)ₙL²(CH₂)ₙ—; L² is —C(═O)NH—; each m is independently 14; and each n is independently an integer from 1 to 6. In embodiments, L₁ is —(CH₂)₃C(═O)NH(CH₂)₅—; and each m is independently an integer from 10 to 16. In embodiments, L₁ is —(CH₂)₃C(═O)NH(CH₂)₅—; and each m is independently an integer from 12 to 16. In embodiments, L₁ is —(CH₂)₃C(═O)NH(CH₂)₅—; and each m is independently an integer from 12 to 14. In embodiments, L₁ is —(CH₂)₃C(═O)NH(CH₂)₅—; and each m is 14.

In embodiments, each m is 12. In embodiments, each m is 13. In embodiments, each m is 14. In embodiments, each m is 15.

In embodiments, provided herein are compounds having the structure of Formula Ie:

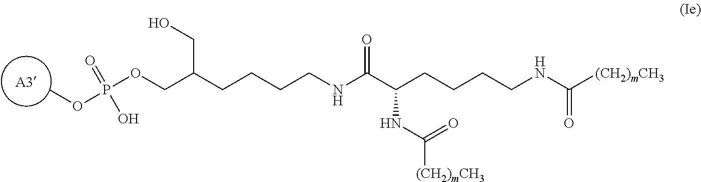

or a pharmaceutically acceptable salt thereof, wherein
A is a modified double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
3′ represents covalent attachment at the terminal 3′ carbon of the sense strand of the modified double stranded nucleic acid;
and m is an integer from 10 to 18.

In embodiments, each m is 12. In embodiments, each m is 13. In embodiments, each m is 14. In embodiments, each m is 15.

The portion of Formula Ie represented by:

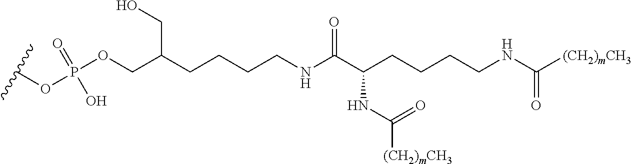

may represent the linker-fatty acid moiety of Formula Ie. In an alternative embodiment, the linker-fatty acid portion of the molecule may be represented by:

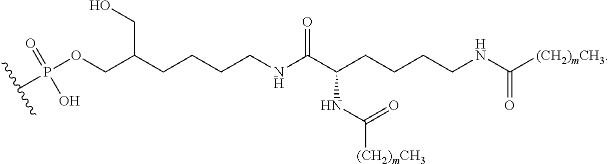

In an alternative embodiment, the linker-fatty acid portion of the molecule may be represented by:

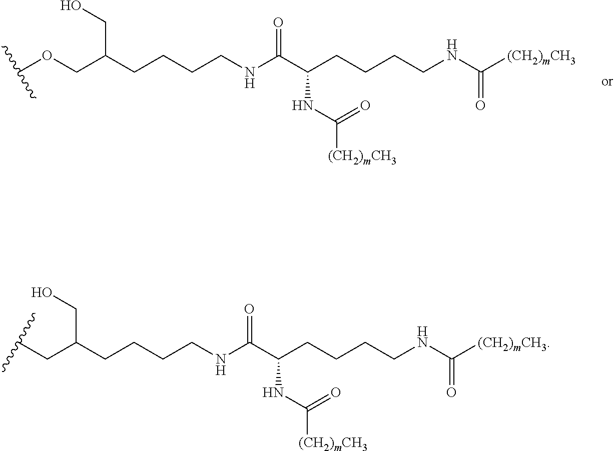

Further embodiments of A are disclosed hereinbefore and throughout the disclosure.

In embodiments, provided herein are compounds having the structure of Formula If:

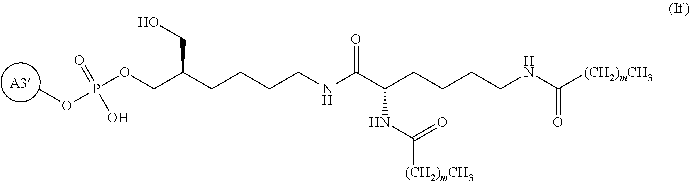

or a pharmaceutically acceptable salt thereof, wherein
A is a modified double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
3′ represents covalent attachment at the terminal 3′ carbon of the sense strand of the modified double stranded nucleic acid;
and m is an integer from 10 to 18.

In embodiments, each m is 12. In embodiments, each m is 13. In embodiments, each m is 14. In embodiments, each m is 15.

The portion of Formula If represented by:

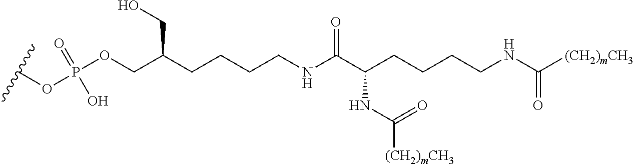

may represent the linker-fatty acid moiety of Formula If. Alternatively, the linker-fatty acid portion of the molecule may be represented by:

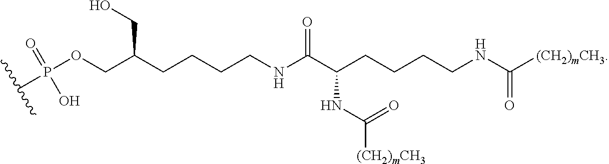

In an alternative embodiment, the linker-fatty acid portion of the molecule may be represented by:

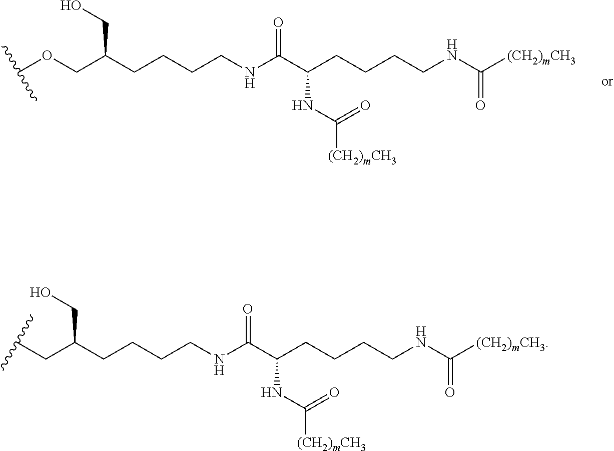

Further embodiments of A are disclosed hereinbefore and throughout the disclosure.

In embodiments, provided herein are compounds having the structure of Formula Ig:

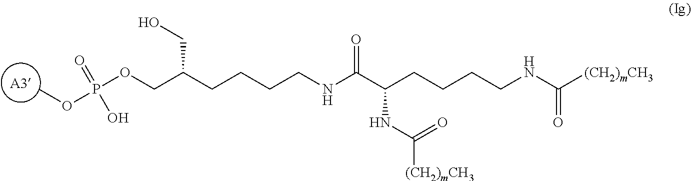

or a pharmaceutically acceptable salt thereof, wherein A is a modified double stranded nucleic acid having suppressing activity of DMPK gene expression, comprising a sense strand and an antisense strand forming a duplex with the sense strand, wherein each of the antisense strand and the sense strand is independently 10 to 30 nucleotides in length;
3′ represents covalent attachment at the terminal 3′ carbon of the sense strand of the modified double stranded nucleic acid;
and m is an integer from 10 to 18.

In embodiments, each m is 12. In embodiments, each m is 13. In embodiments, each m is 14. In embodiments, each m is 15.

The portion of Formula Ig represented by:

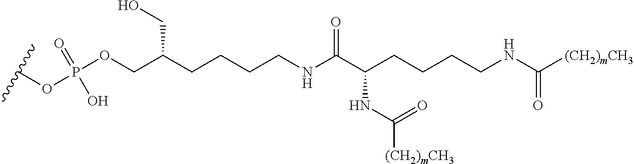

may represent the linker-fatty acid moiety of Formula Ig. Alternatively, the linker-fatty acid portion of the molecule may be represented by:

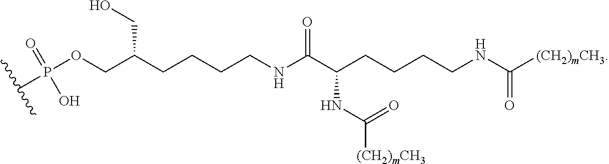

In an alternative embodiment, the linker-fatty acid portion of the molecule may be represented by:

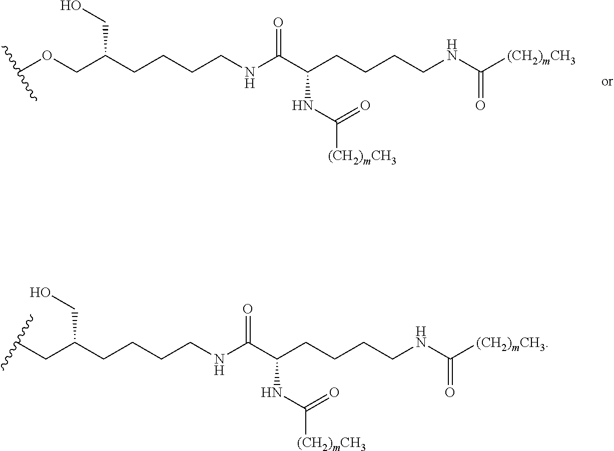

Further embodiments of A are disclosed hereinbefore and throughout the disclosure.

In embodiments of the compounds having the structure of Formula (Ia), (Ib), (Ic), (Id), (Ie), (If), or (Ig) each m is an integer from 10 to 18. In embodiments, each m is an integer from 12 to 14. In embodiments, each m is 10, L₁ is —(CH₂)ₙ—, and n is 3. In embodiments, each m is 11, L₁ is —(CH₂)ₙ—, and n is 3. In embodiments, each m is 12, L₁ is —(CH₂)ₙ—, and n is 3. In embodiments, each m is 13, L₁ is —(CH₂)ₙ—, and n is 3. In embodiments, each m is 14, L₁ is —(CH₂)ₙ—, and n is 3. In embodiments, each m is 15, L₁ is —(CH₂)ₙ—, and n is 3. In embodiments, each m is 16, L₁ is —(CH₂)ₙ—, and n is 3. In embodiments, each m is 17, L₁ is —(CH₂)ₙ—, and n is 3. In embodiments, each m is 18, L₁ is —(CH₂)ₙ—, and n is 3.

In some embodiments, the sense strand comprises at least 17 contiguous nucleotides selected from the nucleotide sequence of any one of SEQ ID Nos: 3 to 293. In some embodiments, the sense strand comprises at least 18 contiguous nucleotides selected from the nucleotide sequence of any one of SEQ ID Nos: 3 to 293. In some embodiments, the sense strand comprises at least 19 contiguous nucleotides selected from the nucleotide sequence of any one of SEQ ID Nos: 3 to 293.

In some embodiments, the dsRNAi agent comprises a structure of

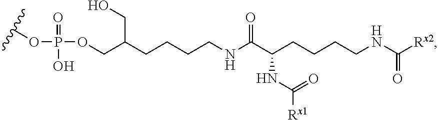

wherein R^{x1} and R^{x2} are each independently C₁₃ alkyl, C₁₄ alkyl, C₁₅ alkyl or C₁₆ alkyl.

In some embodiments, the dsRNAi agent comprises a structure of

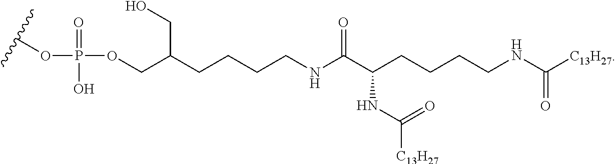

In some embodiments, the dsRNAi agent comprises a structure of

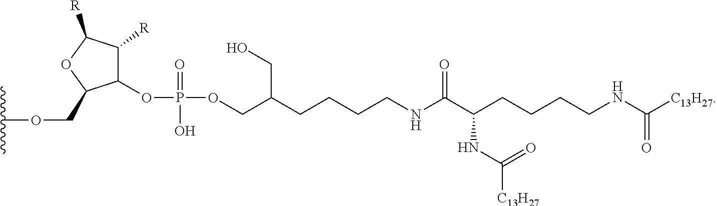

### Methods of Making

The dsRNAi agents of the disclosure can be prepared in a number of ways well known to those skilled in the art of organic synthesis. By way of example, dsRNAi agents of the present disclosure can be synthesized using the methods described below, together with synthetic methods known in the art of synthetic organic chemistry, or variations thereon as appreciated by those skilled in the art. The methods also provide novel intermediates useful in the synthesis of dsRNAi agents of the disclosure.

Generally, the dsRNAi agents of the present disclosure, including the dsRNAi agents of formula (I) and sub formulae thereof, can be prepared according to the Schemes and methods described infra.

The dsRNAi agents of the disclosure can be synthesized using any of a variety of techniques known in the art, such as those described in Usman et al, J. Am. Chem. Soc, 109:7845 (1987); Scaringe et al., Nucl. Acids Res., 18:5433 (1990); Wincott et al., Nucl. Acids Res., 23:2677-2684 (1995); Wincott et al., Methods MoL Bio., 74:59 (1997); and Koster et al., U.S. Pat. No. 4,725,677). The synthesis of oligonucleotides makes use of common nucleic acid protecting and coupling groups, such as dimethoxytrityl at the 5′- end and phosphoramidites at the 3′-end. Suitable reagents for oligonucleotide synthesis, methods for RNA deprotection, and methods for RNA purification are known to those of skill in the art.

Synthesis of dsRNAi agents of the disclosure, e.g., siRNAs, may be prepared using a two-step procedure. First, the individual strands of the double-stranded RNA molecule are prepared separately. Then, the component strands are annealed. The individual strands of the dsRNAi compound can be prepared using solution-phase or solid-phase organic synthesis or both. Organic synthesis offers the advantage that the oligonucleotide strands comprising unnatural or modified nucleotides can be easily prepared. Fatty acid-conjugated dsRNAi agents of the disclosure may be synthesized by the use of an oligonucleotide that bears a pendant reactive functionality, such as that derived from the attachment of a linking molecule onto the oligonucleotide. This reactive oligonucleotide may be reacted directly with commercially available fatty acids, fatty acids that are synthesized bearing any of a variety of protecting groups, or fatty acids that have a linker moiety attached thereto. The methods of the present disclosure facilitate the synthesis of dsRNAi agents by the use of, in some preferred embodiments, nucleoside monomers that may further be attached to a solid-support material.

In the dsRNAi agents and sequence-specific linked nucleosides of the present disclosure, the oligonucleotides and oligonucleosides may be assembled on a suitable DNA synthesizer utilizing standard nucleotide or nucleoside precursors, or nucleotide or nucleoside conjugate precursors that already bear the linking moiety, or ligand-nucleotide or nucleoside conjugate precursors that already bear the linker-fatty acid molecule.

Typical conjugation protocols involve the synthesis of oligonucleotides bearing an amino linker at one or more positions of the sequence. The amino group is then reacted with the molecule being conjugated using appropriate coupling or activating reagents. The conjugation reaction may be performed either with the oligonucleotide still bound to the solid support or following cleavage of the oligonucleotide in solution phase. Purification of the oligonucleotide conjugate by HPLC typically affords the pure conjugate.

Alternatively, the oligonucleotide molecule being conjugated may be converted into a building block such as a phosphoramidite via an alcohol group present in the molecule or by attachment of a linker bearing an alcohol group that may be phosphitylated.

When using nucleotide conjugate precursors that already bear a linking moiety, the synthesis of the sequence-specific linked nucleosides is typically completed, and the ligand molecule, e.g., the fatty acid moiety, is then reacted with the linking moiety to form the ligand conjugated oligonucleotide. The dsRNAi agents of the present disclosure may be prepared by conjugation of the linker or linker-fatty acid molecule to the nucleotide sequence via a reactive functional group on the linker moiety, to link a nucleophilic position, for example, a hydroxyl, thiol, or amino group, on the linker or linker portion of the linker-fatty acid molecule to the nucleotide sequence. This approach to the synthesis of oligonucleotide conjugates bearing a variety of linker-fatty acid motifs has previously been described (see PCT applications WO 2019/232255, WO 2021/108662, WO 2023/086978, WO 2023/086979, and WO 2018/181428, the disclosures of which are herein incorporated by reference in their entirety).

In a particular embodiment the dsRNAi agents of the present disclosure are synthesized by an automated synthesizer using phosphoramidite chemistry that is commercially available and routinely used in oligonucleotide synthesis. For conjugation at the 3′ position, phosphoramidite chemistry is used to introduce the linker-fatty acid moiety, represented herein by -L-FA. Once the linker-fatty acid is coupled, standard oligonucleotide synthesis chemistry is resumed to complete the synthesis of the final sequence, leaving the 5′-ODMT group intact, e.g., oligonucleotide synthesis is carried out in a stepwise fashion using the phosphoramidite chemistry as is known in the art. Following synthesis, global deprotection is carried out using procedures described herein and well-known in the art.

In an aspect, provided herein are methods of making the dsRNAi agents of the disclosure by conjugating a linker-fatty acid moiety to a nucleic acid.

In some embodiments, a linker-fatty acid moiety may be conjugated to the 3′ terminal nucleotide of a nucleic acid, e.g., sense strand or antisense strand. An exemplary process is shown in Scheme I.

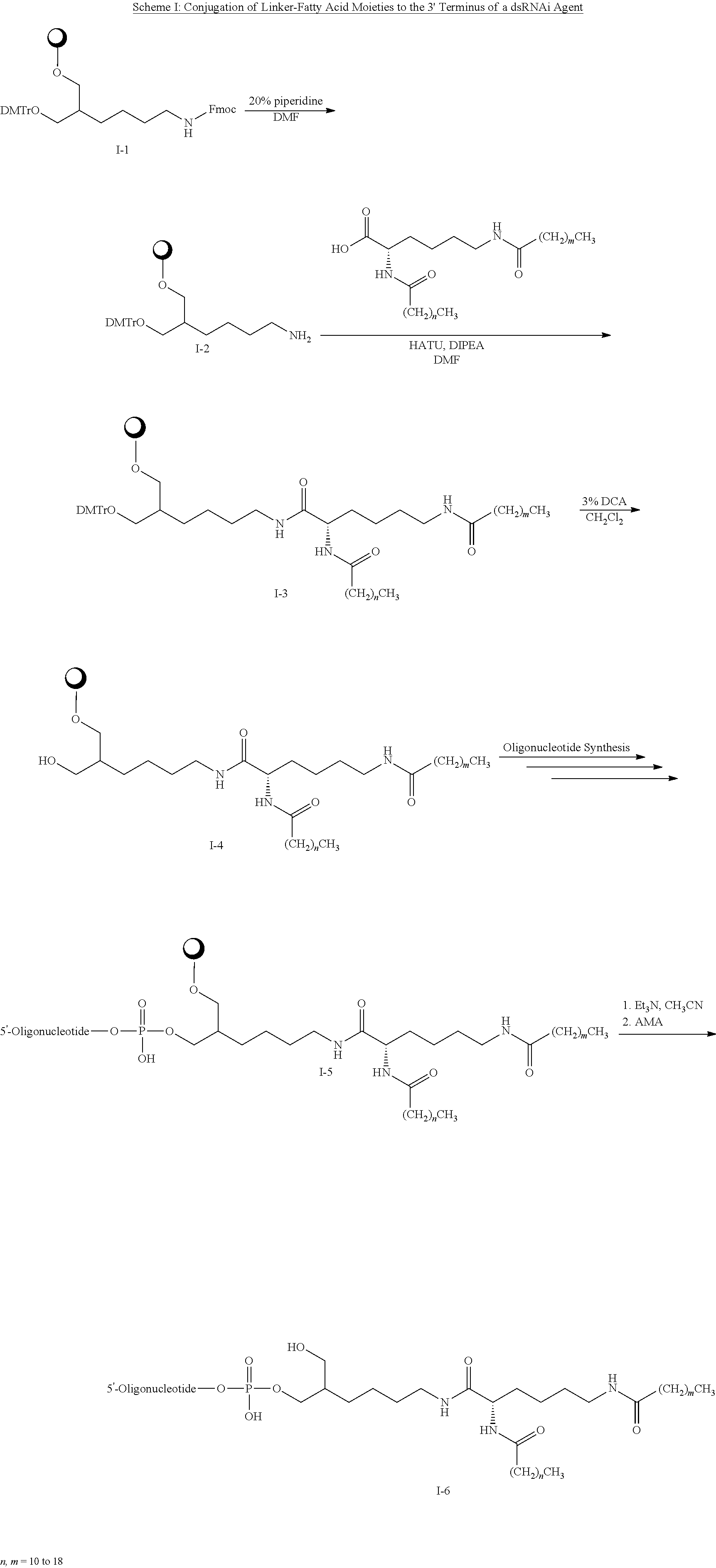

Scheme I above describes the preparation of a dsRNAi agent of the disclosure conjugated with a linker-fatty acid moiety at the 3′ terminus of the oligonucleotide, i.e. at the 3′ carbon of the terminal 3′ nucleotide. For example, 3′-amino CPG beads I-1 (Glen Research, Catalog No. 20-2958) modified with the DMT and Fmoc-protected C₇ linker illustrated above were treated with 20% piperidine/DMF to afford Fmoc-deprotected amino C₇ CPG beads I-2. A linker-fatty acid moiety, e.g. DTx-01-32, was then coupled to I-2 using HATU and DIEA in DMF to produce lipid-loaded CPG beads I-3, which were treated by 3% dichloroacetic acid (DCA) in DCM to remove the DMT protecting group and afford I-4. Oligonucleotide synthesis was accomplished via standard phosphoramidite chemistry and yielded oligonucleotide-bound CPG beads I-5. At this point, if applicable, beads I-5 containing methyl ester-protected lipid motifs, e.g., DTx-01-07-OMe, DTx-01-09-OMe (as disclosed in WO/2019/232255) were saponified to their respective carboxylic acid using 0.5 M LiOH in 3:1 v/v methanol/water. Subsequent treatment of I-5 with AMA [ammonium hydroxide (28%)/methylamine (40%) (1:1, v/v)] cleaved the conjugated oligonucleotide from the beads. The conjugated oligonucleotide was then purified by RP-HPLC and characterized by MALDI-TOF MS using the [M+H] peak.

In some embodiments, a linker-fatty acid moiety may be conjugated to the 5′ terminus, e.g., 5′carbon of the 5′ terminal nucleotide of a nucleic acid, e.g., sense strand or antisense strand. An exemplary process is shown in Scheme II.

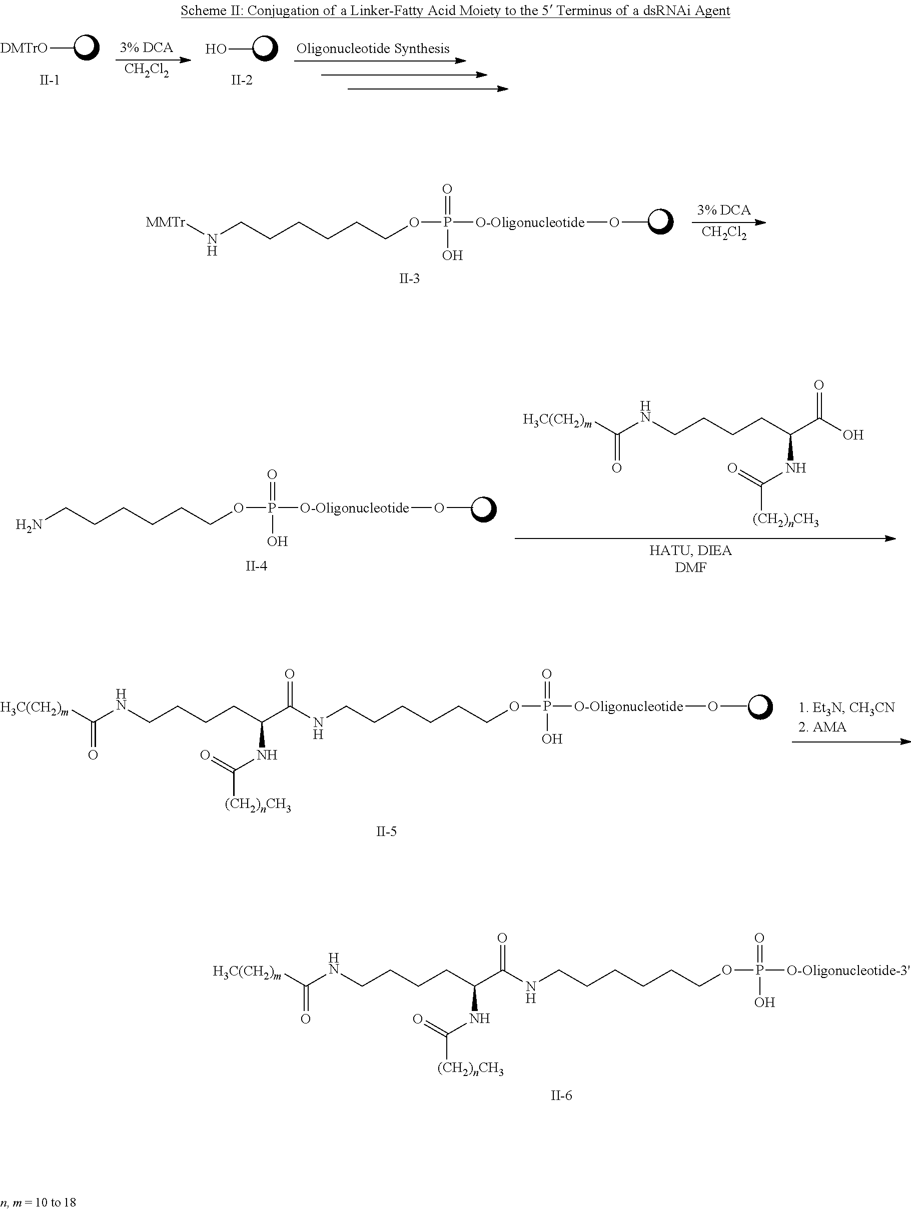

Scheme II above illustrates the preparation of a dsRNAi agent conjugated to a linker-fatty acid moiety at the 5′ terminus, e.g., at the 5′ carbon of the 5′ terminal nucleotide. For example, oligonucleotide synthesis was performed on CPG beads II-1 (Glen Research, Catalog No. 20-5041-xx) via standard phosphoramidite chemistry. In the last nucleotide coupling of the automated sequence, a nucleotide modified with the MMT-protected C₆ linker illustrated above (Glen Research, Catalog No. 10-1906) was used, yielding modified oligonucleotide-bound CPG beads II-3. After removal of MMT with 3% dichloroacetic acid (DCA) in DCM, II-4 was coupled to a fatty acid moiety using HATU and DIEA in DMF to yield II-5. Subsequent treatment with AMA [ammonium hydroxide (28%)/methylamine (40%) (1:1, v/v)] cleaved the conjugated modified oligonucleotide from the beads to generate II-6. The oligonucleotide was then purified by RP-HPLC and characterized by MALDI-TOF MS using the [M+H] peak.

In some embodiments, the linker-fatty acid moiety may be conjugated to both the 3′ terminus and the 5′ terminus, e.g., 3′carbon and 5′ carbon, of the 3′ terminal nucleotide and 5′ terminal nucleotide, respectively, of a nucleic acid, e.g., sense strand and/or antisense strand. An exemplary process is shown in Scheme III.

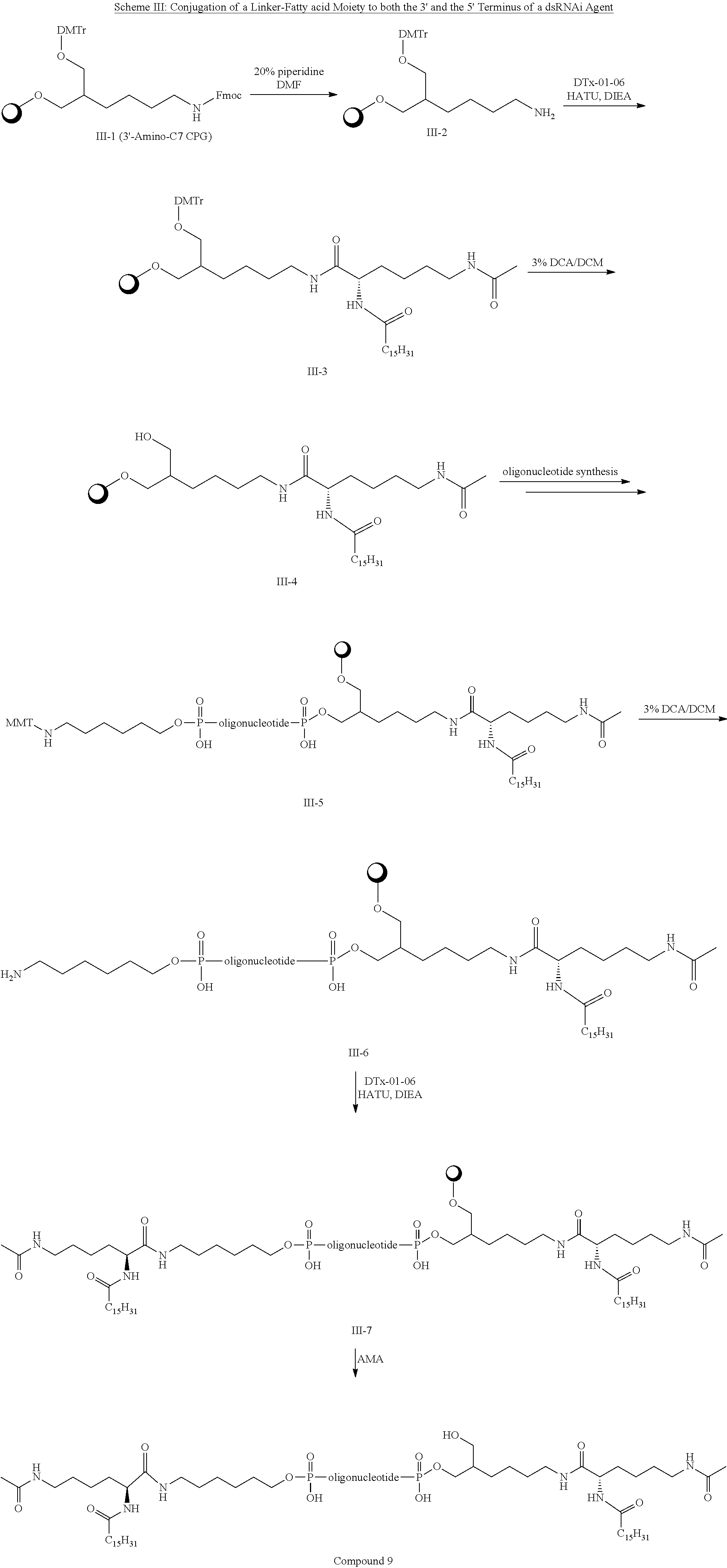

Scheme III above illustrates the preparation of a dsRNAi agent of the disclosure conjugated with linker-fatty acid moieties at both the 3′ and 5′ ends of the passenger or guide strand. In summary, 3′-amino CPG beads III-1 (Glen Research, Catalog No. 20-2958) modified with the DMT and Fmoc-protected C₇ linker illustrated above were treated with 20% piperidine/DMF to afford Fmoc-deprotected amino C₇ CPG beads III-2. Lipid motif DTx-01-06, as disclosed in WO/2019/232255, was then coupled to III-2 using HATU and DIEA in DMF to produce lipid-loaded CPG beads III-3, which were treated by 3% dichloroacetic acid (DCA) in DCM to remove the DMT protecting group and afford III-4. Oligonucleotide synthesis of the passenger or guide strand of the dsRNAi agent as disclosed herein, can be performed on III-4 via standard phosphoramidite chemistry. In the last nucleotide coupling of the automated sequence, a nucleotide modified with the MMT-protected C₆ linker illustrated above (Glen Research, Catalog No. 10-1906), for example, can be used, yielding oligonucleotide-bound CPG beads III-5. After removal of MMT with 3% dichloroacetic acid (DCA) in DCM, III-6 is coupled to DTx-01-06, as disclosed in WO/2019/232255, using HATU and DIEA in DMF to yield III-7. Subsequent treatment of III-7 with AMA [ammonium hydroxide (28%)/methylamine (40%) (1:1, v/v)] cleaves the DTx-01-06-conjugated oligonucleotide from the beads. Compound 9 is then purified by RP-HPLC and characterized by MALDI-TOF MS using the [M+H] peak.

In general, the synthesis of dsRNAi agents disclosed herein starts by using the appropriate coupling cycle, e.g., phosphoramidite coupling cycle. The same coupling cycle can be used to carry out the linker-fatty acid molecule coupling.

In some embodiments, dsRNAi agents having the formula (I), e.g., having the structure:

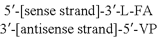

wherein L and FA are defined herein, and VP is the vinyl phosphonate group, e.g., (E)-vinyl phosphonate, are typically synthesized according to the following steps:
(i) providing or synthesizing a solid supported linker-fatty acid molecule comprising a nucleophilic functionality e.g., hydroxyl, amino, thiol, on the linker portion of the linker-fatty acid molecule;
(ii) synthesizing the [sense strand]-3′-L-FA portion of the dsRNAi agent starting from the 3′ end of the sense strand to the 5′ end of the sense strand according to the following steps:
(ii-a) activating a first nucleoside building block of the sense strand at the terminal 3′ end. Preferably, activation is carried out by converting the terminal 3′-OH to a nucleoside-phosphorus acid derivative, e.g., 3′-O—P(N(iPr)₂)(OCH₂CH₂CN), e.g.,

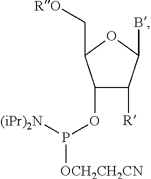

wherein R′ is F or OMe, R″ is a hydroxyl protecting group, e.g., 4,4′-dimethoxytrityl or 4,4′,4′-trimethoxytrityl group, and B′ is the corresponding nucleobase, using a phosphoramidite coupling cycle;
(ii-b) contacting the activated first nucleoside building block from step (ii-a) with the nucleophilic group of the linker-fatty acid molecule under conditions adequate for the reaction between the two functionalities to produce a nucleoside-phosphite ester derivative, e.g., 3′-O—P(L-FA)(OCH₂CH₂CN), e.g.,

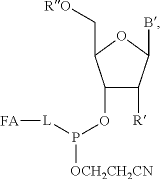

wherein R′ is F or OMe, R″ is a hydroxyl protecting group, e.g., 4,4′-dimethoxytrityl or 4,4′,4′-trimethoxytrityl group, and B′ is the corresponding nucleobase, wherein L and FA are defined herein;
(ii-c) oxidizing the phosphorus of the nucleoside-phosphite ester derivative to a phosphate group, e.g., by oxidation by introduction of O or S;
(ii-d) optionally blocking free primary 5′-OH groups which have not been reacted in any previous step of the phosphoramidite coupling cycle;
(ii-e) introducing further nucleoside phosphate or oligonucleoside phosphate units to produce the desired sense strand sequence;
(ii-f) eliminating all protecting groups and solid supports;
(iii) Providing or synthesizing the 3′-[antisense strand]-5′-VP portion of the dsRNAi agent;
(iv) annealing the sense and antisense strands to produce the dsRNAi agent; and
(v) optionally purifying the resultant dsRNAi agent.

In some embodiments, Step (ii-e) can be repeated to introduce at least one nucleoside phosphate moiety, preferably by one or more phosphoramidite coupling cycles. When oligonucleoside phosphate units are employed, the chains are lengthened by more than one nucleoside phosphate unit.

In an embodiment, provided herein is a process for preparing a compound DT-001068, DT-002211, DT-003035, DT-003036, or DT-003038, comprising the following steps:
(i) synthesizing the linker-fatty acid molecule

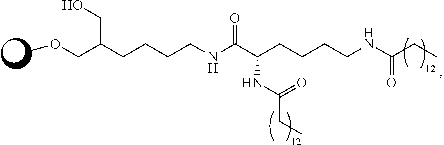

wherein

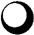

represents a solid support, e.g., controlled pore glass or polystyrene; and
(ii) synthesizing the sense strand portion of DT-001068, DT-002211, DT-003035, DT-003036, or DT-003038, starting from the 3′ end of the sense strand to the 5′ end of the sense strand.

In an embodiment, the process additionally comprises the steps:
(iii) synthesizing the antisense strand portion of DT-001068, DT-002211, DT-003035, DT-003036, or DT-003038, preferably via one or more phosphoramidite coupling cycles;
(iv) annealing the sense and antisense strands to produce DT-001068, DT-002211, DT-003035, DT-003036, or DT-003038; and
(v) optionally purifying the resultant product.

In an embodiment, provided herein is a process for preparing a compound DT-001068, DT-002211, DT-003035, DT-003036, or DT-003038, comprising the following steps:
(i) providing or synthesizing the linker-fatty acid molecule

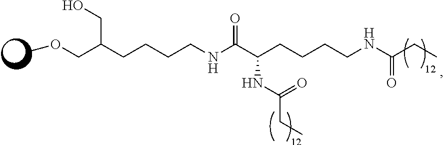

wherein

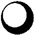

represents a solid support, e.g., controlled pore glass or polystyrene;
(ii) synthesizing the sense strand portion of DT-001068, DT-002211, DT-003035, DT-003036, or DT-003038, starting from the 3′ end of the sense strand to the 5′ end of the sense strand according to the following steps:
(ii-a) reacting a first nucleoside building block

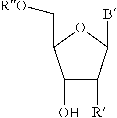

of the DT-001068, DT-002211, DT-003035, DT-003036, or DT-003038 sense strand, starting from the terminal 3′ end, wherein R′ is F or OMe, R″ is a hydroxyl protecting group, e.g., 4,4′-dimethoxytrityl or 4,4′,4′-trimethoxytrityl group, and B′ is the corresponding nucleobase, with a phosphine derivative of formula (P-1)

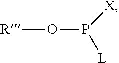

wherein X is chlorine, bromine, CN or SCN; L is chlorine, bromine, CN, SCN or an amino radical of the formula N(R^{P-1})₂, wherein the groups RP-1 denote primary, or secondary or tertiary alkyl radicals having 1-10 carbon atoms, or together denote a cycloalkyl radical having 5-7 carbon atoms, optionally with alkyl branches, and/or can contain one or two nitrogen, oxygen and/or sulfur atoms as heteroatoms, preferably L is N(iPr)₂; R′″ is a group of

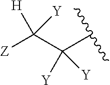

the general formula Y which can be removed with the aid of bases by β-elimination, and in which Y denotes hydrogen, methyl or ethyl, wherein Z represents an electron-attracting group, for example, halogen, such as fluorine, chlorine or bromine, CN or NO₂, preferably R′″ is

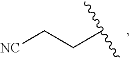

in the presence of a base to produce a nucleotide phosphite compound of formula

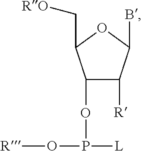

wherein R′, R″, B′ R′″, and L are defined above;
(ii-b) reacting the nucleotide phosphite

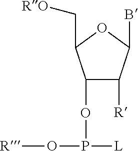

obtained in step (ii-a) with the linker-fatty acid molecule obtained in step (i) under conditions adequate for the reaction between the compounds to produce a solid supported nucleoside-linker-fatty acid

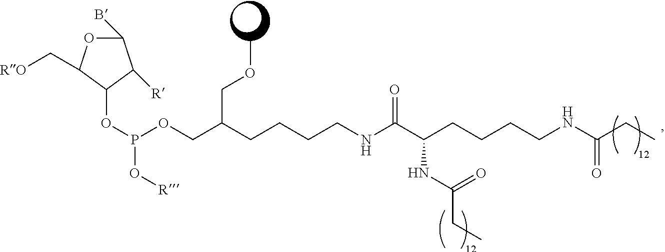

wherein R′, R″, B′, and R′″, are as defined above, with formation of a phosphotriester group;
(ii-c) oxidizing the phosphorus of the solid supported nucleoside-linker-fatty acid obtained in step (ii-b) to a phosphate group, by oxidation by introduction of O;
(ii-d) optionally blocking free primary OH groups which have not been reacted in any previous step;
(ii-e) removing the 5′-hydroxyl protecting group R″ of the solid supported nucleoside-linker-fatty acid obtained in step (ii-c);
(ii-f) introducing further nucleoside phosphate or oligonucleoside phosphate units to produce the desired sense strand sequence, preferably via one or more phosphoramidite coupling cycles;
(ii-g) cleaving the linker solid support bond and eliminating all protecting groups present, e.g., in the oligonucleoside phosphates;
(iii) providing or synthesizing the antisense strand portion of DT-001068, DT-002211, DT-003035, DT-003036, or DT-003038, preferably via phosphoramidite coupling cycles;
(iv) annealing the sense and antisense strands to produce DT-001068, DT-002211, DT-003035, DT-003036, or DT-003038; and
(v) optionally purifying the resultant product.

In some embodiments, dsRNAi agents of formula (I) having the structure

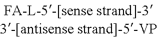

wherein L and FA are defined herein, and VP is a vinyl phosphonate group, e.g., (E)-vinyl phosphonate, are typically synthesized according to the following steps:
   (i) synthesizing the [sense strand] portion of the dsRNAi agent starting from the 3′ end of the sense strand to the 5′ end of the sense strand according to the following steps:
   (i-a) attaching a solid support to the 3′-OH end of a first nucleoside building block of the sense strand; and
   (i-b) synthesizing the sense strand from the solid supported 3′ end to the 5′ end to produce a sense strand of formula HO-5′-[sense strand]-3′-solid support. Preferably, synthesis of the sense strand is carried out by one or more phosphoramidite coupling cycles;
   (ii) activating the 5-OH group of the sense strand of step (i-b). Preferably, activation is carried out by converting the terminal 5′-OH to a nucleoside-phosphorus acid derivative, e.g., (CNCH₂CH₂O)P(N(iPr)₂)—O-5′-[sense strand]-3′-solid support;
   (iii) contacting the activated sense strand from step (ii) with a nucleophilic group of the linker molecule under conditions adequate for the reaction between the two functionalities to produce a nucleoside-phosphite ester derivative, e.g., (L)(NCCH₂CH₂O)—P—O-5′-[sense strand]-3′-solid support, wherein L is a linker as defined herein;
   (iv) oxidizing the nucleoside-phosphorus acid derivative of step (iii) to a phosphate group, e.g., by oxidation by introduction of O or S;
   (v) optionally deprotecting any protecting group or reactive functionality on the linker to allow for further reaction;
   (vi) reacting a reactive functionality, e.g., nucleophilic functionality, e.g., amino group, on the linker portion, e.g., L, of the product of step (iv) or (v) with a fatty acid moiety precursor FA-X¹, wherein the X¹ group has complementary reactivity to the reactive functionality on the linker, under conditions wherein the complementary functional groups react to form a covalent linkage between said linker and fatty acid moiety, e.g., to produce a compound (FA-L)(NCCH₂CH₂O)(O═)P—O-5′-[sense strand]-3′-solid support;
   (vii) eliminating all protecting groups and solid supports;
   (viii) providing or synthesizing the 3′-[antisense strand]-5′-VP portion of the dsRNAi agent;
   (ix) annealing the sense and antisense strands to produce the dsRNAi agent; and
   (x) optionally purifying the resultant dsRNAi agent.

The linker is selected to have reactive functionality at different sites on said linkers in order to provide for a range of orientations of said linker on dsRNAi agents of the disclosure. Such reactive functionality includes, by way of example, carboxylic acids, carboxylic acid halides, carboxyl esters, amines, halides, pseudohalides, isocyanates, vinyl unsaturation, ketones, aldehydes, thiols, alcohols, anhydrides, boronates, and precursors thereof. It is understood, of course, that the reactive functionality on the linker is selected to be complementary to at least one of the reactive groups on the fatty acid moiety so that a covalent linkage can be formed between the linker and the fatty acid moiety.

In some embodiments, dsRNAi agents having the formula (I), e.g., having the structure:

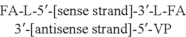

wherein L and FA are defined herein, and VP is the vinyl phosphonate group, e.g., (E)-vinyl phosphonate, are typically synthesized according to the following steps:
(i) providing or synthesizing a solid supported linker-fatty acid molecule comprising a nucleophilic functionality e.g., hydroxyl, amino, thiol, on the linker portion of the linker-fatty acid molecule;
(ii) synthesizing the FA-L-5′-[sense strand]-3′-L-FA portion of the dsRNAi agent starting from the 3′ end of the sense strand to the 5′ end of the sense strand according to the following steps:
(ii-a) activating a first nucleoside building block of the sense strand at the terminal 3′ end. Preferably, activation is carried out by converting the terminal 3′-OH to a nucleoside-phosphorus acid derivative, e.g., 3′-O—P(N(iPr)₂)(OCH₂CH₂CN), e.g.,

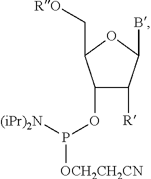

wherein R′ is F or OMe, R″ is a hydroxyl protecting group, e.g., 4,4′-dimethoxytrityl or 4,4′,4′-trimethoxytrityl group, and B′ is the corresponding nucleobase, using a phosphoramidite coupling cycle;
(ii-b) contacting the activated first nucleoside building block from step (ii-a) with the nucleophilic group of the solid supported linker-fatty acid molecule under conditions adequate for the reaction between the two functionalities to produce a nucleoside-phosphite ester derivative, e.g., 3′-O—P(L-FA)(OCH₂CH₂CN), e.g.,

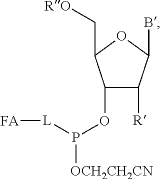

wherein R′ is F or OMe, R″ is a hydroxyl protecting group, e.g., 4,4′-dimethoxytrityl or 4,4′,4′-trimethoxytrityl group, and B′ is the corresponding nucleobase, wherein L and FA are defined herein;
(ii-c) oxidizing the phosphorus of the nucleoside-phosphite ester derivative to a phosphate group, e.g., by oxidation by introduction of 0 or S;
(ii-d) optionally blocking free primary 5′-OH groups which have not been reacted in any previous step of the phosphoramidite coupling cycle;
(ii-e) introducing further nucleoside phosphate or oligonucleoside phosphate units to produce the desired sense strand sequence;
(iii) activating the terminal 5′-OH group of the sense strand of step (ii-e). Preferably, activation is carried out by converting the terminal 5′-OH to a nucleoside-phosphorus acid derivative, e.g., (CNCH₂CH₂O)P(N(iPr)₂)—O-5′-[sense strand]-3′-L-FA;
(iv) contacting the activated sense strand from step (iii) with a nucleophilic group of the linker molecule under conditions adequate for the reaction between the two functionalities to produce a nucleoside-phosphite ester derivative, e.g., (L)(NCCH₂CH₂O)—P—O-5′-[sense strand]-3′-L-FA, wherein L is a linker as defined herein;
(v) oxidizing the nucleoside-phosphorus acid derivative of step (iv) to a phosphate group, e.g., by oxidation by introduction of 0 or S;
(vi) optionally deprotecting any protecting groups or reactive functionality on the linker to allow for further reaction;
(vii) reacting a reactive functionality, e.g., nucleophilic functionality, e.g., amino group, on the linker portion of the product of step (v) or (vi) with a fatty acid moiety precursor FA-X¹, wherein the X¹ group has complementary reactivity to the reactive functionality on the linker, under conditions wherein the complementary functional groups react to form a covalent linkage between said linker and fatty acid moiety, e.g., to produce a compound (FA-L)(NCCH₂CH₂O)(O═)P—O-5′-[sense strand]-3′-L-FA;
(viii) eliminating all protecting groups and solid supports;
(ix) providing or synthesizing the 3′-[antisense strand]-5′-VP portion of the dsRNAi agent;
(x) annealing the sense and antisense strands to produce the dsRNAi agent; and
(xi) optionally purifying the resultant dsRNAi agent.

In an aspect, provided herein are compounds of formula (X), or a salt thereof,

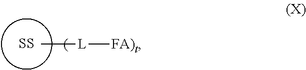

wherein:
SS is a sense strand of the double stranded nucleic acid A, as defined herein, e.g., according to any one of enumerated embodiments 1 to 184;
L is a linker as defined herein, e.g., according to any one of enumerated embodiments 1 to 184;
FA is a fatty acid moiety as defined herein, e.g., according to any one of enumerated embodiments 1 to 184; and
t is an integer from 1 to 5.

In an embodiment, the linker binds at the 3′-end and/or 5′-end of the sense strand SS. In an embodiment, t is 1, 2 or 3.

In an embodiment, the linker and/or sense strand is bound to a solid support, e.g., a controlled pore glass (CPG). A controlled pore glass typically consists of a glass matrix prepared uniformly with pores of defined size. In an embodiment, the linker and/or sense strand is not bound to a solid support.

In an embodiment of formula (X), the formula (X) has the formula (X-1),

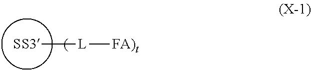

wherein
SS is the sense strand, as defined above, and 3′ means that the linker L binds at the terminal 3′-end of the sense strand;
and t is 1.

In an embodiment of formula (X), the formula (X) has the formula (X-1a),

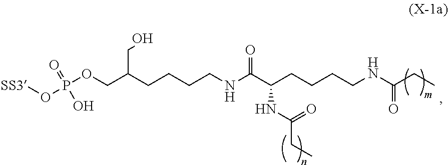

wherein
SS is the sense strand, as defined above; 3′ denotes covalent attachment at the terminal 3′carbon of the sense strand; n is an integer from 10 to 18; and m is an integer from 10 to 18.

In embodiments, both n and m are 12.

In embodiments, SS is selected from a siRNA sense strand nucleic acid sequence in Table 1, Table 2, Table 8, Table 9 or Table 11.

In an embodiment, SS is SEQ ID NO: 258. In an embodiment, SS is SEQ ID NO: 282. In an embodiment, SS is SEQ ID NO: 717. In an embodiment, SS is SEQ ID NO: 840. In an embodiment, SS is identical to a nucleotide sequence selected from SEQ ID NO: 1267, 1311, 1327, 1345, 1347, and 1351. In an embodiment, SS is identical to SEQ ID NO: 1351.

In an embodiment of formula (X), the formula (X) has the formula (X-2),

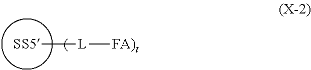

wherein
SS is the sense strand, as defined above, and 5′ means that the linker L binds at the 5′-end of the sense strand;
and t is 1.

Further embodiments of SS are as defined above for compounds of formula (X), including (X-1) and (X-1a).

In an aspect, provided herein are compounds of formula (Y), or a salt thereof,

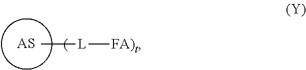

wherein:
AS is an antisense strand of the double stranded nucleic acid A, as defined herein, e.g., according to any one of enumerated embodiments 1 to 184;
L is a linker, as defined herein, e.g., according to any one of enumerated embodiments 1 to 184;
FA is a fatty acid moiety, as defined herein, e.g., according to any one of enumerated embodiments 1 to 184; and
t is an integer from 1 to 5.

In an embodiment, the linker binds at the 3′-end and/or 5′-end of the antisense strand AS. In an embodiment, t is 1, 2 or 3.

In an embodiment, the linker and/or antisense strand is bound to a solid support, e.g., a controlled pore glass (CPG). A controlled pore glass typically consists of a glass matrix prepared uniformly with pores of defined size. In an embodiment, the linker and/or antisense strand is not bound to a solid support.

In an embodiment, the formula (Y) has the formula (Y-1),

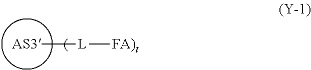

wherein
AS is the sense strand, as defined above, and 3′ means that the linker L binds at the 3′-end of the antisense strand;
and t is 1.

In an embodiment, the formula (Y) has the formula (Y-2),

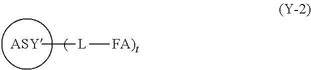

wherein
AS is the antisense strand, as defined above, and 5′ means that the linker L binds at the 5′-end of the antisense strand;
and t is 1.

In some embodiments, provided herein is a compound of formula 1-5, 1-6, II-3, II-4, II-5, II-6, III-5, III-6, III-7 and Compound 9, wherein oligonucleotide denotes the sense strand or the antisense strand of the double stranded nucleic acid A, as defined herein, e.g., according to any one of enumerated embodiments 1 to 184, and n and m are each independently integers from 10 to 18.

In a further embodiment herein is provided a process for the preparation of a dsRNAi agent of formula (I), in free form or in pharmaceutically acceptable salt form, according to any of General Schemes 1, 2 and 3.

Compounds of formulae (X), (X-1), (X-1a), (X-2), (Y), (Y-1), and (Y-2) as defined herein are useful in the preparation of compounds of the disclosure, e.g., dsRNAi compounds of formula (I). Thus, in an aspect, the disclosure relates to a compound of formula (X), (X-1), (X-1a), (X-2), (Y), (Y-1), or (Y-2), or salts thereof. In another aspect, the disclosure relates to the use of a compound of formula (X), (X-1), (X-1a), (X-2), (Y), (Y-1), or (Y-2), or salts thereof, in the manufacture of a dsRNAi agent of formula (I). The disclosure further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure material.

In some embodiments, the linker-fatty acid moiety is conjugated to the 3′ carbon of the 3′ terminal nucleotide of a sense strand described in Table 1. In some embodiments, the linker-fatty acid moiety is conjugated to the 3′ carbon of the 3′ terminal nucleotide of a sense strand disclosed in Table 2.

In some embodiments, the linker-fatty acid moiety is conjugated to the 3′ carbon of the 3′ terminal nucleotide of a sense strand selected from SEQ ID NO: 840. In some embodiments, the linker-fatty acid moiety is conjugated to the 3′ carbon of the 3′ terminal nucleotide of a sense strand selected from SEQ ID NO: 864.

In some embodiments, the linker-fatty acid moiety is conjugated to the 5′ carbon of the 5′ terminal nucleotide of a sense strand described in Table 1. In some embodiments, the linker-fatty acid moiety is conjugated to the 5′ carbon of the 5′ terminal nucleotide of a sense strand disclosed in Table 2.

In some embodiments, the linker-fatty acid moiety is conjugated to the 5′ carbon of the 5′ terminal nucleotide of a sense strand selected from SEQ ID NO: 840. In some embodiments, the linker-fatty acid moiety is conjugated to the 3′ carbon of the 3′ terminal nucleotide of a sense strand selected from SEQ ID NO: 864.

In some embodiments, the linker-fatty acid moiety is conjugated to the 3′ carbon of the 3′ terminal nucleotide of a sense strand described in Table 1. In some embodiments, the linker-fatty acid moiety is conjugated to the 3′ carbon of the 3′ terminal nucleotide of a sense strand disclosed in Table 2.

In some embodiments, the linker-fatty acid moiety is conjugated to the 3′ carbon of the 3′ terminal nucleotide of a sense strand selected from SEQ ID NO: 840. In some embodiments, the linker-fatty acid moiety is conjugated to the 3′ carbon of the 3′ terminal nucleotide of a sense strand selected from SEQ ID NO: 864.

In some embodiments, the linker-fatty acid moiety is conjugated to the 5′ carbon of the 5′ terminal nucleotide of an antisense sense strand described in Table 1. In some embodiments, the linker-fatty acid moiety is conjugated to the 5′ carbon of the 5′ terminal nucleotide of an antisense strand disclosed in Table 2.

In some embodiments, the linker-fatty acid moiety is conjugated to the 5′ carbon of the 5′ terminal nucleotide of an antisense strand selected from SEQ ID NO: 1131. In some embodiments, the linker-fatty acid moiety is conjugated to the 3′ carbon of the 3′ terminal nucleotide of an antisense strand selected from SEQ ID NO: 1155.

In certain aspects, provided is a dsRNAi agent comprising:
(i) an antisense strand of SEQ ID NO: 1268, and
(ii) a sense strand of SEQ ID NO: 1267.

In certain aspects, provided is a dsRNAi agent comprising:
(i) an antisense strand of SEQ ID NO: 1312, and
(ii) a sense strand of SEQ ID NO: 1311.

In certain aspects, provided is a dsRNAi agent comprising:
(i) an antisense strand of SEQ ID NO: 1346, and
(ii) a sense strand of SEQ ID NO: 1345.

In certain aspects, provided is a dsRNAi agent comprising:
(i) an antisense strand of SEQ ID NO: 1348, and
(ii) a sense strand of SEQ ID NO: 1347.

In certain aspects, provided is a dsRNAi agent comprising:
(i) an antisense strand of SEQ ID NO: 1352, and
(ii) a sense strand of SEQ ID NO: 1351.

In some embodiments, the dsRNAi agent consists of:
(i) an antisense strand of SEQ ID NO: 1268, and
(ii) a sense strand of SEQ ID NO: 1267.

In some embodiments, the dsRNAi agent comprises or consists of:
(i) an antisense strand, which is 5′-X033U1027p001A007p001G004p000A007p000C004p000A007p000A004p000U007p000A004p000A007p000A004p000U007p000A004p000C007p000C004p000G007p000A004p000G007p000G004p001A004p001A004-3′ (SEQ ID NO: 1268), and
(ii) a sense strand, which is 5′-C007p001C004p001U007p000C004p000G007p000G004p000U007p000A004p000U007p000U 004p000U007p000A004p000U007p000U004p000G007p000U004p000C007p001U004p001A007p000X2140-3′ (SEQ ID NO: 1267),
wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

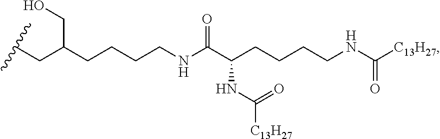

or a pharmaceutically acceptable salt thereof.

In some embodiments, the dsRNAi agent consists of:
(i) an antisense strand of SEQ ID NO: 1312, and
(ii) a sense strand of SEQ ID NO: 1311, or a pharmaceutically acceptable salt thereof.

In some embodiments, the dsRNAi agent comprises or consists of:
(i) an antisense strand, which is 5′-X033U1027p001C007p001A004p000A007p000U004p000A007p000A004p000A007p000U004p000A007p000U004p000C007p000C004p000A007p000A004p000A007p000C004p000C007p000G004p001C004p001C004-3′ (SEQ ID NO: 1312), and
(ii) a sense strand, which is 5′-C007p001G004p001G007p000U004p000U007p000U004p000G007p000G004p000A007p000U 004p000A007p000U004p000U007p000U004p000A007p000U004p000U007p001G004p001A007p000X2140-3′ (SEQ ID NO: 1311),
wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

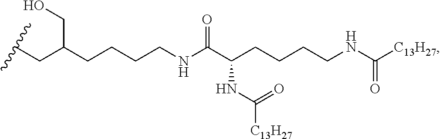

or a pharmaceutically acceptable salt thereof.

In some embodiments, the dsRNAi agent consists of:
(i) an antisense strand of SEQ ID NO: 1346, and
(ii) a sense strand of SEQ ID NO: 1345, or a pharmaceutically acceptable salt thereof.

In some embodiments, the dsRNAi agent comprises or consists of:
(i) an antisense strand, which is 5′-X033U1027p001C007p001A004p000A007p000U004p000A007p000A004p000A007p000U004p000A007p000U004p000C007p000C004p000A007p000A004p000A007p000C004p000C004p000G004p001C004p001C004-3′ (SEQ ID NO: 1346), and
(ii) a sense strand, which is 5′-C004p001G004p001G004p000U004p000U007p000U004p000G007p000G004p000A007p000U 004p000A007p000U004p000U007p000U004p000A007p000U004p000U004p001G004p001A004p000X2140-3′ (SEQ ID NO: 1345),
wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

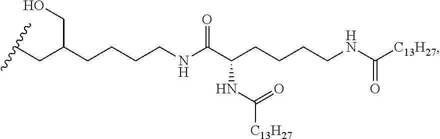

or a pharmaceutically acceptable salt thereof.

In some embodiments, the dsRNAi agent consists of:
(i) an antisense strand of SEQ ID NO: 1348, and
(ii) a sense strand of SEQ ID NO: 1347, or a pharmaceutically acceptable salt thereof.

In some embodiments, the dsRNAi agent comprises or consists of:
(i) an antisense strand, which is 5′-X033U1027p001C007p001A004p000A004p000U004p000A007p000A004p000A004p000U004p000A007p000U004p000C007p000C004p000A007p000A004p000A007p000C004p000C004p000G004p001C004p001C004-3′ (SEQ ID NO: 1348), and
(ii) a sense strand, which is 5′-C004p001G004p001G004p000U004p000U007p000U004p000G004p000G007p000A007p000U 004p000A007p000U004p000U007p000U004p000A004p000U004p000U004p001G004p001A004p000X2140-3′ (SEQ ID NO: 1347),
wherein p000 is a phosphate internucleoside linkage, p001 is a phosphorothioate internucleoside linkage, X033U1027 is 5′-(E)-vinylphosphonate-2′-O-methyluridine, C004 is 2′—O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

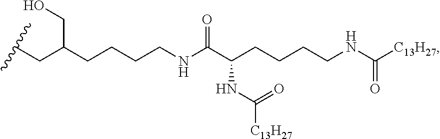

or a pharmaceutically acceptable salt thereof.

In some embodiments, the dsRNAi agent consists of:
(i) an antisense strand of SEQ ID NO: 1352, and
(ii) a sense strand of SEQ ID NO: 1351, or a pharmaceutically acceptable salt thereof.

In some embodiments, the dsRNAi agent comprises or consists of:
(i) an antisense strand, which is 5′-X033U1027p001C007p001A004p000A004p000U004p000A007p000A004p000A004p000U004p000A004p000U004p000C004p000C004p000A007p000A004p000A007p000C004p000C004p000G004p001C004p001C004-3′ (SEQ ID NO: 1352), and
(ii) a sense strand, which is 5′-C004p001G004p001G004p000U004p000U007p000U004p000G007p000G007p000A007p000U 004p000A004p000U004p000U004p000U004p000A004p000U004p000U004p001G004p001U004p000X2140-3′ (SEQ ID NO: 1351),
wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

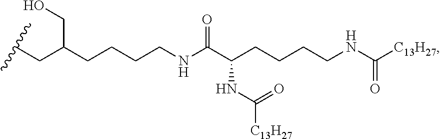

or a pharmaceutically acceptable salt thereof.

In an aspect, provided is a dsRNAi agent comprising a siRNA in Table 8. In an aspect, provided is a dsRNAi agent that includes a siRNA in Table 9. In an aspect, provided is a dsRNAi agent that includes a siRNA in Table 11. In some embodiments, the dsRNAi agent is DT-001068 as described in the disclosure. In some embodiments, the dsRNAi agent is DT-002211 as described in the disclosure. In some embodiments, the dsRNAi agent is DT-003035 as described in the disclosure. In some embodiments, the dsRNAi agent is DT-003036 as described in the disclosure. In some embodiments, the dsRNAi agent is DT-003038 as described in the disclosure.

### Pharmaceutically Acceptable Salts and Compositions

The compounds provided herein may be present as a pharmaceutically acceptable salt. In some embodiments, the dsRNAi agent is a free acid. In some embodiments, the dsRNAi agent is in a salt form, e.g., a pharmaceutically acceptable salt form. It will be understood that references to dsRNAi agent are meant to also include the pharmaceutically acceptable salts of the dsRNAi agent. In some embodiments, the pharmaceutically acceptable salt is a sodium salt.

In some embodiments, a non-bridging heteroatom, e.g., an S— or O—, of a linkage of a compound provided herein may be protonated or associated with a counterion such as Na⁺, K⁺, etc. An acceptable salt, e.g. a pharmaceutically acceptable salt, of a compound may comprise fewer cationic counterions (such as Na⁺, K⁺, etc.) than there are non-bridging heteroatoms per molecule, i.e., some non-bridging heteroatoms are protonated and some are associated with counterions. In some embodiments, a phosphate linkage attaching an -L³-L⁴- to a carbon of a nucleotide includes a non-bridging heteroatom. In some embodiments, a phosphodiester linkage of a nucleic acid includes a non-bridging heteroatom. In some embodiments, a phosphorothioate linkage of a nucleic acid includes a non-bridging heteroatom.

The dsRNAi agents provided herein may be present as a pharmaceutical composition comprising the dsRNAi agent and a pharmaceutically acceptable diluent. In some embodiments, the dsRNAi agent is present in a pharmaceutically acceptable diluent. In some embodiments, the pharmaceutically acceptable diluent is a sterile aqueous solution. In some embodiments, the sterile aqueous solution is a sterile saline solution.

The pharmaceutical composition may be prepared and administered in a wide variety of dosage formulations. The dsRNAi agents described herein may be administered orally, rectally, or by injection, e.g., intravenously, intramuscularly, intracutaneously, subcutaneously, intraduodenally, or intraperitoneally. In certain embodiments, the dsRNAi agents are formulated for injection, e.g., intravenously, intramuscularly, intracutaneously, subcutaneously, intraduodenally, or intraperitoneally. In certain embodiments, the dsRNAi agents are administered subcutaneously. In certain embodiments, the dsRNAi agents are administered intravenously.

For preparing pharmaceutical compositions from the dsRNAi agents described herein, the pharmaceutically acceptable carriers or excipients may be liquid. Particularly, when parenteral application, e.g., subcutaneously or intravenously administered, is needed or desired, particularly suitable admixtures for the compounds included in the pharmaceutical composition may be injectable, sterile solutions, oily or aqueous solutions, as well as suspensions, emulsions, or implants, including suppositories. In certain aspect, for parenteral injection, e.g., subcutaneous or intravenous administration, liquid form preparations may include solutions, e.g., aqueous polyethylene glycol solution, suspensions, emulsions, e.g., water/propylene glycol solutions, aqueous or crystalline compositions, liposomal formulations, and micellar formulations.

The pharmaceutically acceptable carriers or excipients can include buffers to adjust the pH to a desirable range for intravenous use. In some embodiments, the liquid form preparations for intravenous use may include a buffer solution containing acetate, citrate, prolamine, carbonate, phosphate, borate, sulfate, or any combination thereof, for example, the buffer solution is phosphate buffered saline (PBS). In some embodiments, the buffer solution may further include an agent to control the osmolarity, for example, proteins, peptides, amino acids, non-metabolized polymers, vitamins, ions, sugars, metabolites, organic acids, lipids, or salts, e.g., sodium chloride or potassium chloride.

In some embodiments, pharmaceutical compositions containing a dsRNAi agent described herein include additional components to aid in delivery, stability, efficacy, or reduction of immunogenicity.

The pharmaceutical composition may include compositions wherein the active ingredient dsRNAi agent is contained in a therapeutically effective amount, i.e., in an amount effective to achieve its intended purpose, e.g., gene-silencing (e.g., inhibiting, downregulating, or suppressing of the gene), the expression of DMPK in a subject. The actual amount effective for a particular application will depend, inter aim, on the condition being treated.

The pharmaceutical compositions described herein typically include a therapeutically effective amount of a dsRNAi agent described herein. In some embodiments, a therapeutically effective amount of the dsRNAi agent targeting DMPK, e.g., human DMPK, can reduce DMPK mRNA levels in a treated cell or subject by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% compared to a non-treated or control cell or subject. In some embodiments, a therapeutically effective amount of an RNAi agent targeting DMPK can reduce DMPK protein levels in a treated cell or subject by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% compared to non-treated or control cell or subject.

A given clinical treatment is considered effective where there is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% reduction in a measurable parameter associated with a disease or disorder. In some embodiments, the given clinical treatment is considered effective where there is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% reduction in a measurable parameter associated with a disease or disorder. In some embodiments, a therapeutically effective amount of a dsRNAi agent for the treatment of that disease or disorder, e.g., lowering mutant DMPK mRNA levels in disease relevant tissue, e.g., skeletal muscle tissue, of a subject, is the amount necessary to effect at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90% or at least about 95% reduction, respectively, in that parameter. In some embodiments, a therapeutically effective amount of a dsRNAi agent for the treatment of that disease or disorder, e.g., lowering mutant DMPK mRNA levels in disease relevant tissue, e.g., skeletal muscle tissue, of a subject, is the amount necessary to effect at least about 40%, at least about 45%, at least about 50%, at least about 55%, at least about 60%, at least about 65%, at least about 70%, at least about 75%, at least about 80%, at least about 85%, or at least about 90%, respectively, in that parameter.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Dosage amounts and intervals can be adjusted individually to provide levels of the administered dsRNAi agents that is effective for the particular clinical indication being treated. This will provide a therapeutic regimen that is commensurate with the severity of the individual's disease state.

The effective prophylactic or therapeutic treatment regimen as described herein can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

### Methods of Use

The methods and compositions of the present disclosure, e.g., methods and DMPK RNAi agent compositions, can be used in any appropriate dosage and/or composition described herein or known in the art, as well as with any suitable route of administration described herein or known in the art. Any aspects or embodiments disclosed herein that are not mutually exclusive can be combined.

Having regard to their activity as having suppressing activity of DMPK expression, dsRNAi agents of the disclosure, including compounds of formulae (I), (II), (Ia), (Ib), (Ic), (Id), (Ie), (If), and (Ig) as disclosed herein, can be used to treat a subject, e.g., a human or other mammal, having a disease or disorder that would benefit from administration of the dsRNAi agent. In some embodiments, the dsRNAi agents disclosed herein can be used to treat a subject, e.g., a human, that would benefit from a reduction and/or inhibition in expression of DMPK mRNA.

In some embodiments, the dsRNAi agents disclosed herein can be used to treat a subject, e.g., a human, having a disease or disorder for which the subject would benefit from reduction in DMPK mRNA levels, more specifically a reduction in mutant DMPK mRNA levels, including but not limited to, for example, myotonic dystrophy type 1. In some embodiments, the dsRNAi agents disclosed herein can help delay the onset of DM1. Treatment of a subject can include therapeutic and/or prophylactic treatment. The subject is administered a therapeutically effective amount of any one of the dsRNAi agents described herein. The subject can be a human, patient, or human patient. The subject may be an adult, adolescent, child, or infant. Administration of a pharmaceutical composition described herein can be to a human being or animal.

In some embodiments, the described dsRNAi agents are used to treat at least one symptom mediated at least in part by DMPK mRNA levels, e.g., mutant DMPK mRNA levels, in a subject. In some embodiments, the described dsRNAi agents are used to treat at least one symptom mediated at least in part by DMPK protein levels, in a subject. The subject is administered a therapeutically effective amount of any one or more of the described dsRNAi agents. In some embodiments, the subject is administered a prophylactically effective amount of any one or more of the described dsRNAi agents, thereby treating the subject by preventing or inhibiting the at least one symptom.

In some embodiments, the present disclosure provides methods for treatment of diseases, disorders, conditions, or pathological states mediated at least in part by DMPK gene expression, in a patient in need thereof, wherein the methods include administering to the patient any of the dsRNAi agents described herein. In some embodiments, the dsRNAi agents are used to treat or manage a clinical presentation or pathological state in a subject, wherein the clinical presentation or pathological state is mediated at least in part by DMPK expression. The subject is administered a therapeutically effective amount of a dsRNAi agent or pharmaceutical composition comprising said dsRNAi agent described herein. In some embodiments, the method comprises administering a pharmaceutical composition comprising a dsRNAi agent described herein to a subject to be treated.

In some embodiments, the gene expression level or mRNA level of a DMPK gene in certain skeletal muscle cells of a subject to whom a described dsRNAi agent is administered is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, e.g., about 96%, about 97%, about 98%, about 99%, or greater than 99%, relative to the subject prior to being administered the dsRNAi agent or to a subject not receiving the dsRNAi agent. In some embodiments, the DMPK protein levels (including mutant DMPK-CUG protein levels) of a subject to whom a described dsRNAi agent is administered is reduced by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, e.g., about 96%, about 97%, about 98%, about 99%, or greater than 99%, relative to the subject prior to being administered the dsRNAi agent or to a subject not receiving the dsRNAi agent. The gene expression level, protein level, and/or mRNA level in the subject may be reduced in a cell, group of cells, tissue, and/or other fluid of the subject. In some embodiments, the DMPK mRNA levels in certain skeletal muscle cells or skeletal muscle tissues in a subject to whom a described dsRNAi agent has been administered is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, relative to the subject prior to being administered the dsRNAi agent or to a subject not receiving the dsRNAi agent. In some embodiments, the level of DMPK protein (including mutant DMPK-CUG protein levels) in the skeletal muscle cells and/or skeletal muscle tissue of a subject to whom a described compound has been administered is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, relative to the subject prior to being administered the compound or to a subject not receiving the compound.

As noted herein the DMPK protein level (including mutant DMPK-CUG protein levels) and/or DMPK mRNA level in the subject may be reduced in a cell, group of cells, tissue, blood, and/or other fluid (e.g., serum) of the subject, as would be understood by the person of ordinary skill in the art. For example, in some embodiments, the level of DMPK mRNA of a subject to whom a described dsRNAi agent has been administered is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, e.g., about 96%, about 97%, about 98%, about 99%, or greater than 99%, relative to the subject prior to being administered the dsRNAi agent or to a subject not receiving the dsRNAi agent in one or more skeletal muscle cells or skeletal muscle tissues. In some embodiments, the level of DMPK mRNA in a subset of skeletal muscle cells, of a subject to whom a described dsRNAi agent has been administered is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, e.g., about 96%, about 97%, about 98%, about 99%, or greater than 99%, relative to the subject prior to being administered the compound or to a subject not receiving the dsRNAi agent.

In some embodiments, the dsRNAi agents disclosed herein can reduce DMPK gene expression in one or more of the following muscle tissues: heart, biceps, triceps, semitendinosus, platysma, gluteus, tibialis anterior, quadriceps, gastrocnemius, rectus femoris, soleus, extensor digitorum longus, trapezius, diaphragm, and/or tongue.

In embodiments of any of the methods disclosed herein, DMPK gene expression is inhibited by at least about 40%, at least about 50%, at least about 60%, at least about 70%, at least about 80%, or at least about 90%, relative to a control, e.g., when measured by RT-PCR, e.g., RT-qPCR. In embodiments of any of the methods disclosed herein, DMPK mRNA levels are inhibited by at least about 40%, at least about 50%, at least about 60%, at least about 70%, at least about 80%, or at least about 90%, relative to a control, e.g., when measured by RT-PCR, e.g., RT-qPCR.

A reduction in gene expression, mRNA, and protein levels can be assessed by any methods known in the art. For example, the Examples set forth herein provide appropriate ways for measuring DMPK mRNA levels in a subject. The Examples set forth herein illustrate known methods for assessing inhibition of DMPK gene expression.

Cells, tissues, organs, and non-human organisms that include at least one of the DMPK RNAi agents described herein are contemplated. The cell, tissue, organ, or non-human organism is made by delivering the RNAi agent to the cell, tissue, organ, or non-human organism.

Thus, in an aspect, provided herein is a method for inhibiting the expression of DMPK mRNA in a cell. The method includes contacting a cell with a compound provided herein, thereby inhibiting the expression of DMPK in the cell. In some embodiments, the cell is a muscle cell. In some embodiments, the cell is in vivo. In some embodiments, the cell is in vitro. In some embodiments, the cell is a skeletal muscle cell.

In an aspect, provided herein is a method for inhibiting the expression of DMPK in a subject in need thereof, the method comprising administering to the subject an effective amount of a compound of the disclosure, or a pharmaceutically acceptable salt thereof, or pharmaceutical composition provided herein.

In another aspect, provided herein is a method for reducing or preventing aggregation of one or more DMPK mRNA transcripts within a cell, the method comprising contacting a compound of the disclosure, or a pharmaceutically acceptable salt thereof, with the cell. In some embodiments, the cell is a muscle cell. In some embodiments, the cell is a skeletal muscle cell.

In another aspect, provided herein is a method for treating myotonic dystrophy type I in a subject in need thereof, the method comprising administering to the subject an effective amount of a compound of the disclosure, or a pharmaceutically acceptable salt thereof. In some embodiments, the administration of the compound relieves at least one symptom of DM1 in at least one muscle.

In another aspect, provided herein is a method for delaying the onset of myotonic dystrophy type I in a subject in need thereof, the method comprising administering to the subject an effective amount of a compound of the disclosure, or a pharmaceutically acceptable salt thereof. In some embodiments, the administration of the compound relieves at least one symptom of DM1 in at least one muscle.

In another aspect, provided herein is a method for treating myotonic dystrophy type 1 (DM1) in a subject in need thereof, the method comprising systemically administering to the subject an effective amount of a compound of the disclosure, or a pharmaceutically acceptable salt thereof, wherein the administration of the compound relieves at least one symptom of DM1 in at least one muscle.

In embodiments, the at least one symptom of DM1 is a symptom affecting one or more skeletal muscles. In one embodiment, the symptom is selected from the group consisting of myotonia, muscle weakness, muscle atrophy, and muscle pain.

In embodiments, the muscle is selected from the group consisting of skeletal muscle, smooth muscle, and cardiac muscle. In embodiments, the muscle is selected from the group consisting of rectus femoris, triceps, biceps, quadriceps, soleus, EDL (extensor digitorum longus), diaphragm, tibialis anterior, gastrocnemius, heart, trapezius, diaphragm and tongue.

In embodiments of any of the methods disclosed herein, the at least one symptom of DM1 is aggregation of musclebind-like-1 (MBNL-1) protein in ribonuclear foci within myonuclei. In another embodiment, the at least one symptom of DM1 is abnormal splicing of at least one RNA transcript in muscle cells. In some embodiments, the RNA transcript is selected from the group consisting of Serca-1, m-Titin, Zasp, and CIC-1.

In some embodiments, the methods disclosed herein include administering to the subject a therapeutically effective amount of a dsRNAi agent selected from Tables 1-2 and 8-11, or a pharmaceutical composition thereof.

In some embodiments, the methods disclosed herein include administering to the subject a therapeutically effective amount of a dsRNAi agent selected from DT-001068, DT-002211, DT-003035, DT-003036, and DT-003038.

In some embodiments, administration is intravenous administration. In some embodiments, the administration is subcutaneous administration.

In some embodiments, compounds provided herein are for use in therapy. In some embodiments, pharmaceutical compositions provided herein are for use in therapy. In some embodiments, the therapy is the treatment of myotonic dystrophy type I.

All the aforementioned embodiments and embodiments hereinafter relating to the methods of use are equally applicable to:
A dsRNAi agent as described herein, for use in such methods, including for use in the treatment of DM1, according to the present disclosure;
Use of a dsRNAi agent as described herein in such methods, including for use in the treatment of DM1, according to the present disclosure;
Use of a dsRNAi agent as described herein, in the manufacture of a medicament for such methods, including for use in the treatment of DM1, according to the present disclosure;
A pharmaceutical composition comprising a dsRNAi agent as described herein, for use in such methods, including for use in the treatment of DM1, according to the present disclosure;
Use of a pharmaceutical composition comprising a dsRNAi agent as described herein in such methods, including for use in the treatment of DM1, according to the present disclosure; and
Use of a pharmaceutical composition comprising a dsRNAi agent as described herein, in the manufacture of a medicament for such methods, including for the treatment of DM1, according to the present disclosure.

### Formulations

Various formulations are available to facilitate compound use both in vitro and as therapeutic agents. Accordingly, In some embodiments, a compound provided herein is present in a formulation.

Compounds may be formulated with cationic lipids to facilitate transfection into cells. Suitable cationic lipid reagents for transfection include Lipofectamine reagents, such as Lipofectamine RNAiMAX.

For use in vivo as therapeutic agents, nucleic acids compounds may be encapsulated into lipid nanoparticles. Lipid nanoparticles generally comprise a cationic lipid, a non-cationic lipid, and a lipid that prevents aggregation of the nanoparticle. Suitable cationic lipids include D-Lin-MC3-DMA ((6Z,9Z,28Z,31Z)-Heptatriaconta-6,9,28,31-tetraen-19-yl 4-(dimethylamino)butanoate), DLin-KC2-DMA (2,2-dilinoleyl-4-dimethylaminoethyl-[1,3]-dioxolane) and the lipidoid C12-200. Suitable non-cationic lipids include, for example, DOPC (1,2-dioleoyl-sn-glycero-3-phosphatidylcholine) and DSPC (1,2-distearoyl-sn-glycero-3-phosphocholine). Examples of lipids that prevent aggregation include, for example, polyethylene glycol (PEG)-lipids, such as PEG-c-DMA (3-N-[(ω-methoxypoly(ethylene glycol)2000)carbamoyl]-1,2-dimyristyloxy-propylamine), PEG(2000)-C-DMG (α-(3-{[1,2-di(myristyloxy)proponoxy]carbonylamino}propyl)-ω-methoxy, polyoxyethylene), and mPEG-DSPE (1,2-distearoyl-sn-glycero-3-phosphoethanolamine-N-[methoxy(polyethylene glycol)-2000]).

### EXAMPLES

The following examples are presented to more fully illustrate some embodiments of the disclosure. They should not be construed, however, as limiting the scope of the disclosure. Variations of these examples within the scope of the claims are within the purview of one skilled in the art and are considered to fall within the scope of the embodiments as described and claimed herein. The reader will recognize that the skilled artisan, armed with the present disclosure and skill in the art, is able to prepare and use the disclosure without exhaustive examples.

### Example 1. Synthesis of Linkers and Fatty Acid Moieties and Conjugation to Oligonucleotides

Linkers and fatty acid moieties were synthesized and conjugated to oligonucleotides as described in International Patent Application Publication No. WO 2019/232255, the disclosure of which is herein incorporated by reference in its entirety. Certain synthesis methods and conjugation schemes are described below.

### Synthesis of Lipid Motif DTx-01-32

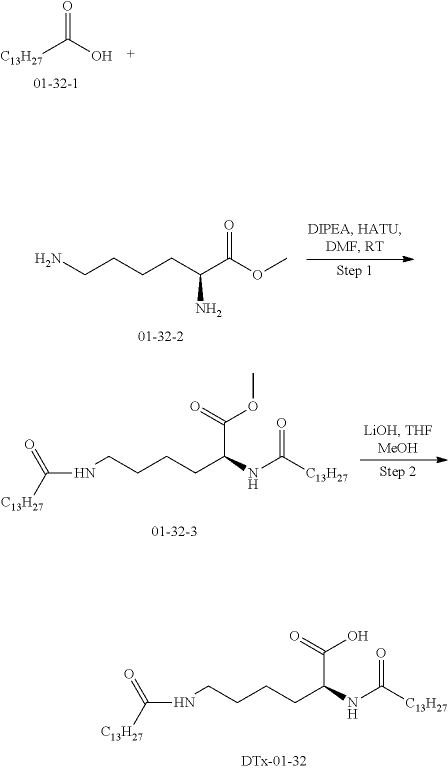

### Step 1: Synthesis of Intermediate 01-32-3

To a stirred solution of 01-32-2 (3 g, 0.01 mol) in DMF (50 mL) at RT was added slowly DIPEA (13.8 mL, 0.077 mol), linear fatty acid 01-32-1 (4.4 g, 0.0154 mol), and HATU (5.87 g, 0.0154 mol). The resulting mixture was stirred at 60° C. After 16 h, the reaction mixture was quenched with ice water, the solids isolated by filtration, and the solids dried under vacuum to afford 01-32-3 as an off-white solid (3.5 g, 53.2%).

### Step 2: Synthesis of Lipid Motif DTx-01-32

To a stirred solution of 01-32-3 (3.5 g, 0.0051 mol) in MeOH (10 mL), THF (10 mL), and water (3 mL), was added LiOH·H₂O (0.8 g, 0.0154 mol). The reaction mixture was stirred 16 h. Subsequently, the reaction mixture was concentrated under vacuum and neutralized with 1.5 N HCl. The solids were isolated by filtration, washed with water, and dried under vacuum, affording crude DTx-01-32. Recrystallization (80% DCM in hexane) yielded lipid motif DTx-01-32 as an off-white solid (2.3 g, 79.3%). LCMS m/z (M+H)+: 567.2; ¹H-NMR (400 MHz, TFA-d): δ 0.87-0.98 (m, 6H), 1.20-1.58 (m, 41H), 1.74-1.92 (m, 8H), 2.18-2.21 (m, 2H), 2.73 (t, J=7.6 Hz, 2H), 3.05 (t, J=7.6 Hz, 2H), 3.60 (t, J=7.8 Hz, 2H).

### Example 2. Preparation of Oligonucleotides (siRNAs)

Small scale synthesis is used to prepare DMPK siRNAs; medium and large scale syntheses can also be used to prepare these siRNAs in larger quantities.

### Example 2.1. Small Scale Synthesis and Purification Methods for the Initial Screens (1 μMole Scale)

### 1) Synthesis, Cleavage and Deprotection:

The synthesis of DMPK sequences use solid support oligonucleotide synthesis using phosphoramidite chemistry on MerMade 192 synthesizer (BioAutomation, Part of LGC Biosearch Technologies) at 1 & 5 μmole mol scale. The synthesis of the above sequences is performed at 1 & 5 μmole scale in 96 well plates. The RNA, DNA, 2′-OMe and 2′-F phosphoramidite solutions are prepared at 0.1 M concentration and 5-(Ethylthio)tetrazole (0.25 M Acetonitrile) is used as activator. Deblock solution, oxidizer solution and capping solution are prepared according to standard processes.

The synthesized sequences are cleaved and deprotected in 96 well plates, using methylamine solution (a 3:1 mixture of aqueous and ethanolic solutions) or ammonium hydroxide. The crude sequences are purified via anion exchange or reverse phase (or a combination of both) in cartridges or plates. Samples from each sequence are analyzed by LC-MS to confirm the identity, UV for quantification and a selected set of samples by LC-MS chromatography to determine purity.

The linker-fatty acid moiety was introduced at the 3′ terminus of the sense strand of the siRNA using a functionalized solid support, as described in Scheme I. In summary, 3′amino CPG beads I1 (Glen Research, Catalog No. 202958) modified with the DMT and Fmoc protected C7 linker illustrated above were treated with 20% piperidine/DMF to afford Fmoc deprotected amino C7 CPG beads 12. An fatty acid moiety (e.g., DTx-01-32) was then coupled to 12 using HATU and DIEA in DMF to produce lipid loaded CPG beads 13, which were treated by 3% dichloroacetic acid (DCA) in Toluene to remove the DMT protecting group and afford 14. Oligonucleotide synthesis was accomplished via standard phosphoramidite chemistry and yielded oligonucleotide bound CPG beads 15. At this point, if applicable, beads I5 containing methyl ester protected lipid motifs (e.g., DTx-01-09-OMe) were saponified to their respective carboxylic acid using 0.5 M LiOH in 3:1 v/v methanol/water. Subsequent treatment of I5 with AMA [ammonium hydroxide (28%)/methylamine (40%) (1:1, v/v)] or ammonium hydroxide cleaved the conjugated oligonucleotide from the beads. PS linkages were prepared by oxidation of phosphite utilizing 0.1 M (xanthane hydride [5-amino-3H-1,2,4-dithiazole-3-thione] in pyridine. Subsequent treatment of I5 with AMA [ammonium hydroxide (28%)/methylamine (40%) (1:1, v/v)] or ammonium hydroxide cleaved the conjugated oligonucleotide from the beads. The conjugated oligonucleotide was then purified by anion exchange or reverse phase and characterized by LC-MS.

Purification and desalting: DMPK tiled sequences are purified on AKTA explorer purification system using Source 15Q or Source 30 RPC column. Sample injection and collection are performed via fractionation The full length sequence is collected in the eluent. The purified sequences are desalted on a reverse phase column using AKTA purifier of by TFF. The concentration of desalted DMPK sequences are calculated using absorbance at 260 nm wavelength and purity was measured by LC-MS reverse phase chromatography.

Annealing: Purified desalted sense and antisense single strands are mixed in equimolar amounts and annealed to form DMPK duplexes. The duplexes are prepared at 10 uM concentration in 1×PBS buffer, normal saline or water and tested by RP-HPLC for purity. Medium scale synthesis and purification (1-250 μmol).

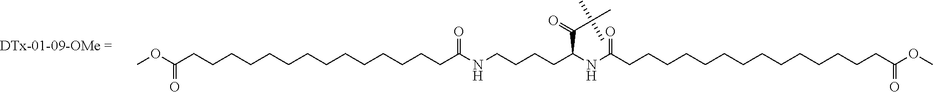

### Example 2.2. Medium Scale Synthesis can Also be Used to Generate siRNAs

Single stranded RNAs in scales between 1 and 250 μmol were prepared by solid phase synthesis using a MerMade 12 synthesizer (BioAutomation, Part of LGC Biosearch Technologies. Universal Support was purchased from AM Chemicals LLC (Vista, CA) or ChemGenes (Wilmington, MA) and 3′-lipid controlled pore glass (CPG) support (500 Å, loading 50-100 μmol/g) were homemade. For larger scales, empty synthesis columns (50-5000 μmol) from Cytiva and large amidite (250 mL) and reagent bottles (2000 mL) were used. RNA and RNA containing 2′-MOE, 2′-F or 2′-O-methyl nucleotides were generated by solid phase synthesis employing the corresponding phosphoramidites (Hongene Biotech Corporation, Union City, CA). These building blocks were incorporated at selected sites within the sequence of the oligoribonucleotide chain using standard nucleoside phosphoramidite chemistry such as described in Current Protocols in Nucleic Acid Chemistry, Beaucage, S. L. et al. (Edrs.), John Wiley & Sons, Inc., New York, NY, USA. Vinylphosphonates at the 5′ end of the antisense strand were introduced by using solid phase synthesis of 5′-(E)-vinylphosphonate-2′-OMe-U phosphoramidite monomers. Phosphorothioate linkages were introduced using a solution of the 0.1 M xanthane hydride [5-amino-3H-1,2,4-dithiazole-3-thione] in pyridine.

The synthesized DMPK sequences were cleaved and deprotected in AMA solution (1:1 mixture of methylamine solution and 40% NH₃ aqueous solutions) for 3.5 hours or in 40% NH₃ aqueous solutions at 55° C. overnight. To deprotect 5′-vinylphosphonated oligonucleotide, additional 3% of diethyl amine was added to deprotection solution. Preparative ion-pair reverse phase liquid chromatography (IPRP-LC) and IEX-HPLC were applied to purified oligonucleotide products. Samples from each sequence were analyzed by LC-MS to confirm the identity, UV absorbance at 260 nm for quantification and a selected set of samples by IPRP-HPLC to determine purity. The identities and purities of all oligonucleotides were confirmed using electrospray ionization mass spectrometry (ESI-MS); Double stranded RNA is generated by mixing an equimolar solution of complementary strands in water or annealing buffer (typically phosphate buffered solution, PBS, Ambion, Applied Biosystems, Austin, TX) at the desired concentration. The mixture was then heated in a water bath at 85-90° C. for 5 minutes and cooled to room temperature over a period of 1-4 hours. The RNA duplex was stored at −20° C. until use.

### Example 2.3. Duplex Formation

For each of the strands synthesized by Schemes I, II, or III and listed above, the corresponding complementary strand was prepared via standard phosphoramidite chemistry, purified by IE-HPLC, and characterized by MALDI-TOF MS using the [M+H] peak. The duplex was formed by mixing equal molar equivalents of the passenger strand (the sense strand) and guide strand (the antisense strand), heating to 90° C. for 5 minutes, and then slowly cooling to room temperature. Duplex formation was confirmed by non-denaturing PAGE or non-denaturing HPLC.

### Example 3: Biology Experimental Methods

Cell Culture. Human Skeletal Muscle Cells (HSkMC) were purchased from Cell Applications and were cultured in HSkMC Growth Media in a humidified 37° C. incubator with 5% CO₂. Human Fibroblast cell lines, isolated from healthy and DM1 patient skin, were purchased from Coriell Institute for Medical Research. Fibroblast cell lines were cultured in MEM containing 15% Fetal Bovine Serum (FBS), 2 mM L-glutamine, 1× non-essential amino acids, 100 U/mL penicillin and 100 mg/mL streptomycin in a humidified 37° C. incubator with 5% CO₂.

Reverse Transfection of siRNA. HSkMCs cells were trypsinized and diluted to 20,000 cells/well, in 90 μL of antibiotic-free cell differentiation media (Cell Applications, #151D-250). Fibroblast cell lines were trypsinized and diluted to 10,000 cells/well, in 90 μL of antibiotic-free media (DMEM containing 15% FBS, 2 mM L-glutamine, 1× non-essential amino acids). Compounds were diluted in PBS to 100× of the desired final concentration. Separately, Lipofectamine RNAiMax (Life Technologies) was diluted 1:66.7 in media lacking supplements (e.g., FBS, antibiotic etc.). The 100× compound in PBS was then complexed with RNAiMAX by adding 1 part compound in PBS to 9 parts lipofectamine/media. Following incubation for 20 minutes, 10 μL of the compound:RNAiMAX complexes were added to a 96-well collagen coated plate. A volume of 90 μL of the cell dilution was added to each well of the 96-well plate. The plate was then placed in a humidified 37° C. incubator with 5% CO₂. After 24 hours, the complexes were removed and replaced with complete media containing antibiotics for each cell line. RNA was isolated 48 hours following transfection.

Free uptake of conjugated siRNA. HEK293 cells were trypsinized and diluted to 20,000 cells/well, in 100 μL of complete media and allowed to settle overnight in 96 well collagen coated plates. Schwann cells were trypsinized and diluted to 10,000 cells/well, in 100 μL of complete media and allowed to settle for 48 hours in 96 well collagen coated plates. Compounds were diluted in deep well plates in the corresponding basal media for each cell line supplemented with 2% FBS to the desired final concentration of the top dose then serially diluted. After the appropriate amount of time for cells to settle, media was removed from plates by inverting. 100 μL of compound or PBS at proper concentrations was added to each well of the 96 well plate. HEK293 cells were incubated for 48 hours, and Schwann cells were incubated 72 hours in a humidified 37° C. incubator with 5% CO₂ before RNA was isolated.

RNA Isolation, Reverse Transcription and Quantitative PCR. RNA was isolated utilizing the RNeasy 96 kit (Qiagen) according to the manufacturer's protocol. RNA was reverse transcribed to cDNA utilizing random primers and the high capacity cDNA reverse transcription kit (ThermoFisher Scientific) in a SimpliAmp thermal cycler (ThermoFisher Scientific) according to the manufacturer's instructions. Real time quantitative PCR was performed utilizing gene specific primers (Thermofisher Scientific; IDTDNA), TaqMan probes (Thermofisher Scientific; IDTDNA) and TaqMan fast universal PCR master mix (Thermofisher scientific) on a QuantStudio 6 real time PCR system (Thermofisher Scientific) according to the manufacturer's instructions. For analysis of quantitative PCR, mRNA expression was normalized to the expression of either 18s rRNA, beta-actin or Rp1p0 mRNA (housekeeping genes) utilizing the relative CT method according to the best practices proposed in Nature Protocols (Schmittgen, T. D. & Livak, K. J. Analyzing real-time PCR data by the comparative C(T) method. Nat Protoc 3, 1101-1108 (2008)).

Mice. DMSXL mice were obtained from the Dr. Genevieve Gourdon laboratory at the Institut de Myologie at the INSERM. DMSXL mice have a transgene consisting of a 45-kb human genomic fragment with the full DMPK gene with ~1300 CTG repeats in the 3′ UTR and the two flanking genes, SIX5 and DMWD. Hemizygous male and female mice were used to set up a mouse colony by breeding with wildtype (C57BL/6J) animals to expand to number of DMSXL hemizygous breeders. Once the hemizygous colony was established, the transgenic line was maintained by breeding hemizygous DMSXL males with hemizygous DMSXL females to produce wildtype, hemizygous, and homozygous offspring. All litters were weaned between 21-23 days of age and tail clipped for genotyping. Both hemizygous and homozygous DMSXL mice of both genders were used for experiments.

Intravenous injection. Mice were weighed the day before the study initiation. On the day of the study, the mice were restrained with an approved device and injected with the treatment of interest (compound or PBS) via the tail vein.

Target Engagement Studies in vivo in DMSXL hemizygous mice. 14-84 days following intravenous injection of the compound of interest or control, the mice were euthanized. Quadriceps, Gastrocnemius, Tibialis anterior, and soleus muscles, as well as heart, diaphragm, and tongue were dissected and prepared for RNA isolation. The regions of interest were placed in tubes containing beads, flash frozen and stored at −80° C. until RNA isolation. To extract total RNA, Trizol was added to the tubes and RNA isolated using the RNeasy 96 kit via the manufacturer's instructions. RNA Isolation, Reverse Transcription and Quantitative PCR were performed as described earlier with primers specific to human DMPK with no reactivity on mouse DMPK.

Target Engagement Studies in vivo in non-human primates (NHP). 28 days following intravenous injection of the compound of interest or control, the NHP were euthanized. Each tissue sample was immersed in 5-10× volumes of RNAlater in a labelled cryotube and incubated at 4° C. overnight, after which the RNAlater was removed and the tissues stored in a freezer set to maintain −80° C. Frozen tissues were pulverized using a Covaris pulverizer. The pulverized tissue when transferred to a clean tube and ceramic beads were added. 500 uL Trizol was added to each tube and samples were homogenized for 1 minute. RNA was isolated using the RNeasy 96 kit via the manufacturer's instructions. RNA Isolation, Reverse Transcription and Quantitative PCR were performed as described earlier with primers specific to NHP DMPK.

RT-PCR analysis of gene splicing. RNA Isolation, Reverse Transcription and Quantitative PCR were performed as described earlier with some modifications. For each splice event, two sets of primer probe assays were run; one assay that detects all variants of the gene of interest (T) and one assay that exclusively detects the exon of interest (E). Each assay was run alongside a housekeeping gene (HK) and both assays were run on the same plate with the same cDNA. Percent spliced in (PSI) was calculated using the following equation:

$$PSI = {2}^{⋀} - ( ( HK Ct - E Ct ) - ( HK Ct - T Ct ) ) ⋆ 100$$

Percent change of exon inclusion compared to vehicle was calculated by normalizing all data points to the average of the vehicle group and multiplying by 100.

$$For each sample : PSI / average PSI for vehicle ⋆ 100$$

Composite splice scores were calculated using a panel of at least three splice defects in each tissue that showed significance between DMSXL homozygous mice and wildtype mice. Composite splice scores were calculated as follows. For each sample “i”, normalized splicing values were calculated for each splice event “t” as (PSIi,t−PSIwildtype,t)/(PSIdmsxl,t−PSIwildtype,t) where PSIwildtype,t is the median PSI for event t across five wildtype mice and PSIdmsxl,t is the median PSI for event t across vehicle treated DMSXL homozygous mice. The composite score was then calculated as the mean of all normalized splicing values. This was repeated for each tissue.

### Example 4. RNA In Situ Hybridization (ISH) to Quantify Foci (RNAscope®)

All samples collected (heart, left Tibialis anterior, gastrocnemius, tongue) were placed inside plastic cassettes, post fixed for 48 hours in 10% normal buffered formalin (Leica, catalog #3800598) and transferred in a fully enclosed tissue processor (Leica ASP300). Thus, samples underwent dehydration by immersing them in a series of ethanol solutions of increasing concentrations. Afterwards, samples were embedded in paraffin, sectioned at a 5 μm thickness and mounted on Superfrost microscope slides (Fisher Scientific, catalog #22-037-246). Mutant DMPK RNA transcripts were stained using the RNAscope® VS Universal HRP Reagent Kit singleplex, chromogenic-brown assay (Advanced Cell Diagnostics, catalog 323200) designed for use with the Discovery Ultra automated slide staining systems by Ventana Medical Systems. Following the manufacture's protocol, we used a nucleic acid probe targeting the expanded CUG repeats of the DMPK sense transcripts (Advanced Cell Diagnostics, catalog #547819). In addition, during each assay, we included tissues stained using probes designed to target the housekeeping Ppib (Advanced Cell Diagnostics, catalog #313919) and dapB (Advanced Cell Diagnostics, catalog #312039) gene transcripts as positive and negative controls, respectively. We used the Discovery mRNA DAB detection kit (Roche, catalog #760-224) to detect the expression of the targets of interest. Nuclear counterstain was achieved using both hematoxylin II (Roche, catalog #790-2208) and bluing reagent (Roche, 760-2037). Slides were cover slipped using Micromount (Leica, catalog #3801731) and left at room temperature overnight before imaging.

Imaging and analysis: Stained slides were scanned using a Leica Aperio AT2. After scanning, 40× images were loaded into Halo software (Indica lab, version 3.5). For each tissue section collected, blind to the experimental conditions, several regions of interest (ROI) were drawn in different portion of the tissue. Based on computer-generated markup images, an algorithm (Halo object colocalization v2.1.5, developed by Indica lab) was used to detect the presence of DAB (brown) staining overlapping with that of hematoxylin II and to assess the % RNA foci-positive nuclei (% of hematoxylin II-positive cells with nuclei showing brown staining) and the total RNA area (μm²) occupied by the brown staining.

### Example 5. Unconjugated siRNAs Targeting DMPK

A library of siRNAs targeting the human DMPK mRNA was designed and synthesized. The sense and antisense strands of the compounds were prepared with sugar moiety and internucleoside linkage modifications to increase hybridization affinity, minimize degradation by nucleases, and enhance loading into RISC.

In this siRNA library, each antisense strand is 100% complementary to a 21-nucleotide region of the nucleotide sequence of the human DMPK mRNA (NCBI Reference Sequence NM_001081563.2, deposited with GenBank on Jan. 4, 2014; SEQ ID NO: 1).

The 5′ and 3′ nucleotide positions of each 21-nucleotide region are shown in Table 1, with “Start” and “End” corresponding to the 5′ and 3′ nucleotide positions, respectively, of the nucleotide sequence of the SEQ ID NO: 1. The 5′ position of the region is complementary to the 3′-terminal nucleotide of the antisense strand, and the 3′ position of the region is complementary to the 5′-terminal nucleotide of the antisense strand. The sense strand is 19 nucleotides in length and is fully complementary to nucleotides 1 to 19 of the antisense strand (counting from the 5′-end of the antisense strand). Thus, each siRNA comprises a double-stranded region of 19 nucleobase pairs and a two-nucleotide overhang at the 3′-end of the antisense strand.

TABLE 1
Unconjugated siRNAs targeting DMPK (unmodified sequences)

| siRNA | | | | SEQ | | SEQ |
|---|---|---|---|---|---|---|
| compound | | | Sense strand | ID | Antisense strand | ID |
| ID | start | end | unmodified sequence | NO. | unmodified sequence | NO. |
| DT- | 752 | 770 | AGGCUUAAGGAGGUCCGAU | 3 | AUCGGACCUCCUUAAGCCUCA | 294 |
| 001531 | | | | | | |
| DT- | 753 | 771 | GGCUUAAGGAGGUCCGACU | 4 | AGUCGGACCUCCUUAAGCCUC | 295 |
| 001532 | | | | | | |
| DT- | 754 | 772 | GCUUAAGGAGGUCCGACUU | 5 | AAGUCGGACCUCCUUAAGCCU | 296 |
| 001533 | | | | | | |
| DT- | 884 | 902 | AAGUGGGACAUGCUGAAGA | 6 | UCUUCAGCAUGUCCCACUUGU | 297 |
| 001534 | | | | | | |
| DT- | 885 | 903 | AGUGGGACAUGCUGAAGAU | 7 | AUCUUCAGCAUGUCCCACUUG | 298 |
| 001535 | | | | | | |
| DT- | 905 | 923 | GGCGAGGUGUCGUGCUUCU | 8 | AGAAGCACGACACCUCGCCCC | 299 |
| 001536 | | | | | | |
| DT- | 983 | 1001 | GCCUUCCAGGAUGAGAACU | 9 | AGUUCUCAUCCUGGAAGGCGA | 300 |
| 001537 | | | | | | |
| DT- | 984 | 1002 | CCUUCCAGGAUGAGAACUA | 10 | UAGUUCUCAUCCUGGAAGGCG | 301 |
| 001538 | | | | | | |
| DT- | 985 | 1003 | CUUCCAGGAUGAGAACUAU | 11 | AUAGUUCUCAUCCUGGAAGGC | 302 |
| 001539 | | | | | | |
| DT- | 988 | 1006 | CCAGGAUGAGAACUACCUA | 12 | UAGGUAGUUCUCAUCCUGGAA | 303 |
| 001540 | | | | | | |
| DT- | 989 | 1007 | CAGGAUGAGAACUACCUGU | 13 | ACAGGUAGUUCUCAUCCUGGA | 304 |
| 001541 | | | | | | |
| DT- | 990 | 1008 | AGGAUGAGAACUACCUGUA | 14 | UACAGGUAGUUCUCAUCCUGG | 305 |
| 001542 | | | | | | |
| DT- | 991 | 1009 | GGAUGAGAACUACCUGUAU | 15 | AUACAGGUAGUUCUCAUCCUG | 306 |
| 001543 | | | | | | |
| DT- | 999 | 1017 | ACUACCUGUACCUGGUCAU | 16 | AUGACCAGGUACAGGUAGUUC | 307 |
| 001544 | | | | | | |
| DT- | 1000 | 1018 | CUACCUGUACCUGGUCAUU | 17 | AAUGACCAGGUACAGGUAGUU | 308 |
| 001545 | | | | | | |
| DT- | 1001 | 1019 | UACCUGUACCUGGUCAUGU | 18 | ACAUGACCAGGUACAGGUAGU | 309 |
| 001546 | | | | | | |
| DT- | 1002 | 1020 | ACCUGUACCUGGUCAUGGA | 19 | UCCAUGACCAGGUACAGGUAG | 310 |
| 001547 | | | | | | |
| DT- | 1051 | 1069 | GCUGAGCAAGUUUGGGGAU | 20 | AUCCCCAAACUUGCUCAGCAG | 311 |
| 001548 | | | | | | |
| DT- | 1056 | 1074 | GCAAGUUUGGGGAGCGGAU | 21 | AUCCGCUCCCCAAACUUGCUC | 312 |
| 001549 | | | | | | |
| DT- | 1106 | 1124 | GAGAUUGUCAUGGCCAUAU | 22 | AUAUGGCCAUGACAAUCUCCG | 313 |
| 001550 | | | | | | |
| DT- | 1107 | 1125 | AGAUUGUCAUGGCCAUAGA | 23 | UCUAUGGCCAUGACAAUCUCC | 314 |
| 001551 | | | | | | |
| DT- | 1142 | 1160 | GGCUACGUGCACAGGGACA | 24 | UGUCCCUGUGCACGUAGCCAA | 315 |
| 001552 | | | | | | |
| DT- | 1143 | 1161 | GCUACGUGCACAGGGACAU | 25 | AUGUCCCUGUGCACGUAGCCA | 316 |
| 001553 | | | | | | |
| DT- | 1144 | 1162 | CUACGUGCACAGGGACAUU | 26 | AAUGUCCCUGUGCACGUAGCC | 317 |
| 001554 | | | | | | |
| DT- | 1145 | 1163 | UACGUGCACAGGGACAUCA | 27 | UGAUGUCCCUGUGCACGUAGC | 318 |
| 001555 | | | | | | |
| DT- | 1146 | 1164 | ACGUGCACAGGGACAUCAA | 28 | UUGAUGUCCCUGUGCACGUAG | 319 |
| 001556 | | | | | | |
| DT- | 1147 | 1165 | CGUGCACAGGGACAUCAAA | 29 | UUUGAUGUCCCUGUGCACGUA | 320 |
| 001557 | | | | | | |
| DT- | 1148 | 1166 | GUGCACAGGGACAUCAAAU | 30 | AUUUGAUGUCCCUGUGCACGU | 321 |
| 001558 | | | | | | |
| DT- | 1149 | 1167 | UGCACAGGGACAUCAAACU | 31 | AGUUUGAUGUCCCUGUGCACG | 322 |
| 001559 | | | | | | |
| DT- | 1692 | 1710 | GGCUCACUGCCAUGGUGAU | 32 | AUCACCAUGGCAGUGAGCCCG | 323 |
| 001560 | | | | | | |
| DT- | 1693 | 1711 | GCUCACUGCCAUGGUGAGU | 33 | ACUCACCAUGGCAGUGAGCCC | 324 |
| 001561 | | | | | | |
| DT- | 1694 | 1712 | CUCACUGCCAUGGUGAGCU | 34 | AGCUCACCAUGGCAGUGAGCC | 325 |
| 001562 | | | | | | |
| DT- | 1695 | 1713 | UCACUGCCAUGGUGAGCGU | 35 | ACGCUCACCAUGGCAGUGAGC | 326 |
| 001563 | | | | | | |
| DT- | 2012 | 2030 | CUCACCCGGCAGAGCCUGA | 36 | UCAGGCUCUGCCGGGUGAGCA | 327 |
| 001564 | | | | | | |
| DT- | 2013 | 2031 | UCACCCGGCAGAGCCUGAU | 37 | AUCAGGCUCUGCCGGGUGAGC | 328 |
| 001565 | | | | | | |
| DT- | 2203 | 2221 | CACGGAUCCACCUUCCCAU | 38 | AUGGGAAGGUGGAUCCGUGGC | 329 |
| 001566 | | | | | | |
| DT- | 2204 | 2222 | ACGGAUCCACCUUCCCAUC | 39 | GAUGGGAAGGUGGAUCCGUGG | 330 |
| 001567 | | | | | | |
| DT- | 2205 | 2223 | CGGAUCCACCUUCCCAUCU | 40 | AGAUGGGAAGGUGGAUCCGUG | 331 |
| 001568 | | | | | | |
| DT- | 2206 | 2224 | GGAUCCACCUUCCCAUCUA | 41 | UAGAUGGGAAGGUGGAUCCGU | 332 |
| 001569 | | | | | | |
| DT- | 2207 | 2225 | GAUCCACCUUCCCAUCUAU | 42 | AUAGAUGGGAAGGUGGAUCCG | 333 |
| 001570 | | | | | | |
| DT- | 2208 | 2226 | AUCCACCUUCCCAUCUAGA | 43 | UCUAGAUGGGAAGGUGGAUCC | 334 |
| 001571 | | | | | | |
| DT- | 2209 | 2227 | UCCACCUUCCCAUCUAGAU | 44 | AUCUAGAUGGGAAGGUGGAUC | 335 |
| 001572 | | | | | | |
| DT- | 2210 | 2228 | CCACCUUCCCAUCUAGAUU | 45 | AAUCUAGAUGGGAAGGUGGAU | 336 |
| 001573 | | | | | | |
| DT- | 2292 | 2310 | ACCUGCUGCUCCCUGCCAU | 46 | AUGGCAGGGAGCAGCAGGUGG | 337 |
| 001574 | | | | | | |
| DT- | 2313 | 2331 | UCCCUAGGCCUGGCCUAUU | 47 | AAUAGGCCAGGCCUAGGGACC | 338 |
| 001575 | | | | | | |
| DT- | 3154 | 3172 | AUUCCUCGGUAUUUAUUGU | 48 | ACAAUAAAUACCGAGGAAUGU | 339 |
| 001576 | | | | | | |
| DT- | 3155 | 3173 | UUCCUCGGUAUUUAUUGUU | 49 | AACAAUAAAUACCGAGGAAUG | 340 |
| 001577 | | | | | | |
| DT- | 3158 | 3176 | CUCGGUAUUUAUUGUCUGU | 50 | ACAGACAAUAAAUACCGAGGA | 341 |
| 001578 | | | | | | |
| DT- | 3159 | 3177 | UCGGUAUUUAUUGUCUGUU | 51 | AACAGACAAUAAAUACCGAGG | 342 |
| 001579 | | | | | | |
| DT- | 3160 | 3178 | CGGUAUUUAUUGUCUGUCU | 52 | AGACAGACAAUAAAUACCGAG | 343 |
| 001580 | | | | | | |
| DT- | 734 | 752 | GCGGAGCCCAUCGUGGUGA | 53 | UCACCACGAUGGGCUCCGCUG | 344 |
| 001581 | | | | | | |
| DT- | 735 | 753 | CGGAGCCCAUCGUGGUGAU | 54 | AUCACCACGAUGGGCUCCGCU | 345 |
| 001582 | | | | | | |
| DT- | 739 | 757 | GCCCAUCGUGGUGAGGCUU | 55 | AAGCCUCACCACGAUGGGCUC | 346 |
| 001583 | | | | | | |
| DT- | 740 | 758 | CCCAUCGUGGUGAGGCUUA | 56 | UAAGCCUCACCACGAUGGGCU | 347 |
| 001584 | | | | | | |
| DT- | 741 | 759 | CCAUCGUGGUGAGGCUUAA | 57 | UUAAGCCUCACCACGAUGGGC | 348 |
| 001585 | | | | | | |
| DT- | 742 | 760 | CAUCGUGGUGAGGCUUAAU | 58 | AUUAAGCCUCACCACGAUGGG | 349 |
| 001586 | | | | | | |
| DT- | 745 | 763 | CGUGGUGAGGCUUAAGGAU | 59 | AUCCUUAAGCCUCACCACGAU | 350 |
| 001587 | | | | | | |
| DT- | 748 | 766 | GGUGAGGCUUAAGGAGGUU | 60 | AACCUCCUUAAGCCUCACCAC | 351 |
| 001588 | | | | | | |
| DT- | 776 | 794 | AGGGACGACUUCGAGAUUU | 61 | AAAUCUCGAAGUCGUCCCUCU | 352 |
| 001589 | | | | | | |
| DT- | 778 | 796 | GGACGACUUCGAGAUUCUU | 62 | AAGAAUCUCGAAGUCGUCCCU | 353 |
| 001590 | | | | | | |
| DT- | 781 | 799 | CGACUUCGAGAUUCUGAAU | 63 | AUUCAGAAUCUCGAAGUCGUC | 354 |
| 001591 | | | | | | |
| DT- | 787 | 805 | CGAGAUUCUGAAGGUGAUU | 64 | AAUCACCUUCAGAAUCUCGAA | 355 |
| 001592 | | | | | | |
| DT- | 791 | 809 | AUUCUGAAGGUGAUCGGAU | 65 | AUCCGAUCACCUUCAGAAUCU | 356 |
| 001593 | | | | | | |
| DT- | 841 | 859 | GAUGAAGCAGACGGGCCAU | 66 | AUGGCCCGUCUGCUUCAUCUU | 357 |
| 001594 | | | | | | |
| DT- | 846 | 864 | AGCAGACGGGCCAGGUGUA | 67 | UACACCUGGCCCGUCUGCUUC | 358 |
| 001595 | | | | | | |
| DT- | 868 | 886 | CAUGAAGAUCAUGAACAAU | 68 | AUUGUUCAUGAUCUUCAUGGC | 359 |
| 001596 | | | | | | |
| DT- | 870 | 888 | UGAAGAUCAUGAACAAGUU | 69 | AACUUGUUCAUGAUCUUCAUG | 360 |
| 001597 | | | | | | |
| DT- | 877 | 895 | CAUGAACAAGUGGGACAUU | 70 | AAUGUCCCACUUGUUCAUGAU | 361 |
| 001598 | | | | | | |
| DT- | 883 | 901 | CAAGUGGGACAUGCUGAAU | 71 | AUUCAGCAUGUCCCACUUGUU | 362 |
| 001599 | | | | | | |
| DT- | 908 | 926 | GAGGUGUCGUGCUUCCGUU | 72 | AACGGAAGCACGACACCUCGC | 363 |
| 001600 | | | | | | |
| DT- | 910 | 928 | GGUGUCGUGCUUCCGUGAU | 73 | AUCACGGAAGCACGACACCUC | 364 |
| 001601 | | | | | | |
| DT- | 915 | 933 | CGUGCUUCCGUGAGGAGAU | 74 | AUCUCCUCACGGAAGCACGAC | 365 |
| 001602 | | | | | | |
| DT- | 922 | 940 | CCGUGAGGAGAGGGACGUU | 75 | AACGUCCCUCUCCUCACGGAA | 366 |
| 001603 | | | | | | |
| DT- | 924 | 942 | GUGAGGAGAGGGACGUGUU | 76 | AACACGUCCCUCUCCUCACGG | 367 |
| 001604 | | | | | | |
| DT- | 930 | 948 | AGAGGGACGUGUUGGUGAA | 77 | UUCACCAACACGUCCCUCUCC | 368 |
| 001605 | | | | | | |
| DT- | 931 | 949 | GAGGGACGUGUUGGUGAAU | 78 | AUUCACCAACACGUCCCUCUC | 369 |
| 001606 | | | | | | |
| DT- | 932 | 950 | AGGGACGUGUUGGUGAAUG | 79 | CAUUCACCAACACGUCCCUCU | 370 |
| 001607 | | | | | | |
| DT- | 960 | 978 | GGUGGAUCACGCAGCUGCA | 80 | UGCAGCUGCGUGAUCCACCGC | 371 |
| 001608 | | | | | | |
| DT- | 981 | 999 | UCGCCUUCCAGGAUGAGAA | 81 | UUCUCAUCCUGGAAGGCGAAG | 372 |
| 001609 | | | | | | |
| DT- | 982 | 1000 | CGCCUUCCAGGAUGAGAAU | 82 | AUUCUCAUCCUGGAAGGCGAA | 373 |
| 001610 | | | | | | |
| DT- | 1003 | 1021 | CCUGUACCUGGUCAUGGAU | 83 | AUCCAUGACCAGGUACAGGUA | 374 |
| 001611 | | | | | | |
| DT- | 1004 | 1022 | CUGUACCUGGUCAUGGAGU | 84 | ACUCCAUGACCAGGUACAGGU | 375 |
| 001612 | | | | | | |
| DT- | 1005 | 1023 | UGUACCUGGUCAUGGAGUA | 85 | UACUCCAUGACCAGGUACAGG | 376 |
| 001613 | | | | | | |
| DT- | 1006 | 1024 | GUACCUGGUCAUGGAGUAU | 86 | AUACUCCAUGACCAGGUACAG | 377 |
| 001614 | | | | | | |
| DT- | 1009 | 1027 | CCUGGUCAUGGAGUAUUAU | 87 | AUAAUACUCCAUGACCAGGUA | 378 |
| 001615 | | | | | | |
| DT- | 1012 | 1030 | GGUCAUGGAGUAUUACGUU | 88 | AACGUAAUACUCCAUGACCAG | 379 |
| 001616 | | | | | | |
| DT- | 1036 | 1054 | GGACCUGCUGACACUGCUU | 89 | AAGCAGUGUCAGCAGGUCCCC | 380 |
| 001617 | | | | | | |
| DT- | 1038 | 1056 | ACCUGCUGACACUGCUGAU | 90 | AUCAGCAGUGUCAGCAGGUCC | 381 |
| 001618 | | | | | | |
| DT- | 1041 | 1059 | UGCUGACACUGCUGAGCAA | 91 | UUGCUCAGCAGUGUCAGCAGG | 382 |
| 001619 | | | | | | |
| DT- | 1042 | 1060 | GCUGACACUGCUGAGCAAU | 92 | AUUGCUCAGCAGUGUCAGCAG | 383 |
| 001620 | | | | | | |
| DT- | 1043 | 1061 | CUGACACUGCUGAGCAAGU | 93 | ACUUGCUCAGCAGUGUCAGCA | 384 |
| 001621 | | | | | | |
| DT- | 1044 | 1062 | UGACACUGCUGAGCAAGUU | 94 | AACUUGCUCAGCAGUGUCAGC | 385 |
| 001622 | | | | | | |
| DT- | 1046 | 1064 | ACACUGCUGAGCAAGUUUA | 95 | UAAACUUGCUCAGCAGUGUCA | 386 |
| 001623 | | | | | | |
| DT- | 1067 | 1085 | GAGCGGAUUCCGGCCGAGA | 96 | UCUCGGCCGGAAUCCGCUCCC | 387 |
| 001624 | | | | | | |
| DT- | 1068 | 1086 | AGCGGAUUCCGGCCGAGAU | 97 | AUCUCGGCCGGAAUCCGCUCC | 388 |
| 001625 | | | | | | |
| DT- | 1077 | 1095 | CGGCCGAGAUGGCGCGCUU | 98 | AAGCGCGCCAUCUCGGCCGGA | 389 |
| 001626 | | | | | | |
| DT- | 1078 | 1096 | GGCCGAGAUGGCGCGCUUA | 99 | UAAGCGCGCCAUCUCGGCCGG | 390 |
| 001627 | | | | | | |
| DT- | 1079 | 1097 | GCCGAGAUGGCGCGCUUCU | 100 | AGAAGCGCGCCAUCUCGGCCG | 391 |
| 001628 | | | | | | |
| DT- | 1080 | 1098 | CCGAGAUGGCGCGCUUCUA | 101 | UAGAAGCGCGCCAUCUCGGCC | 392 |
| 001629 | | | | | | |
| DT- | 1081 | 1099 | CGAGAUGGCGCGCUUCUAU | 102 | AUAGAAGCGCGCCAUCUCGGC | 393 |
| 001630 | | | | | | |
| DT- | 1092 | 1110 | GCUUCUACCUGGCGGAGAU | 103 | AUCUCCGCCAGGUAGAAGCGC | 394 |
| 001631 | | | | | | |
| DT- | 1093 | 1111 | CUUCUACCUGGCGGAGAUU | 104 | AAUCUCCGCCAGGUAGAAGCG | 395 |
| 001632 | | | | | | |
| DT- | 1094 | 1112 | UUCUACCUGGCGGAGAUUA | 105 | UAAUCUCCGCCAGGUAGAAGC | 396 |
| 001633 | | | | | | |
| DT- | 1095 | 1113 | UCUACCUGGCGGAGAUUGU | 106 | ACAAUCUCCGCCAGGUAGAAG | 397 |
| 001634 | | | | | | |
| DT- | 1096 | 1114 | CUACCUGGCGGAGAUUGUU | 107 | AACAAUCUCCGCCAGGUAGAA | 398 |
| 001635 | | | | | | |
| DT- | 1098 | 1116 | ACCUGGCGGAGAUUGUCAU | 108 | AUGACAAUCUCCGCCAGGUAG | 399 |
| 001636 | | | | | | |
| DT- | 1099 | 1117 | CCUGGCGGAGAUUGUCAUG | 109 | CAUGACAAUCUCCGCCAGGUA | 400 |
| 001637 | | | | | | |
| DT- | 1104 | 1122 | CGGAGAUUGUCAUGGCCAU | 110 | AUGGCCAUGACAAUCUCCGCC | 401 |
| 001638 | | | | | | |
| DT- | 1105 | 1123 | GGAGAUUGUCAUGGCCAUA | 111 | UAUGGCCAUGACAAUCUCCGC | 402 |
| 001639 | | | | | | |
| DT- | 1155 | 1173 | GGGACAUCAAACCCGACAA | 112 | UUGUCGGGUUUGAUGUCCCUG | 403 |
| 001640 | | | | | | |
| DT- | 1156 | 1174 | GGACAUCAAACCCGACAAU | 113 | AUUGUCGGGUUUGAUGUCCCU | 404 |
| 001641 | | | | | | |
| DT- | 1157 | 1175 | GACAUCAAACCCGACAACA | 114 | UGUUGUCGGGUUUGAUGUCCC | 405 |
| 001642 | | | | | | |
| DT- | 1158 | 1176 | ACAUCAAACCCGACAACAU | 115 | AUGUUGUCGGGUUUGAUGUCC | 406 |
| 001643 | | | | | | |
| DT- | 1159 | 1177 | CAUCAAACCCGACAACAUU | 116 | AAUGUUGUCGGGUUUGAUGUC | 407 |
| 001644 | | | | | | |
| DT- | 1165 | 1183 | ACCCGACAACAUCCUGCUU | 117 | AAGCAGGAUGUUGUCGGGUUU | 408 |
| 001645 | | | | | | |
| DT- | 1168 | 1186 | CGACAACAUCCUGCUGGAU | 118 | AUCCAGCAGGAUGUUGUCGGG | 409 |
| 001646 | | | | | | |
| DT- | 1173 | 1191 | ACAUCCUGCUGGACCGCUU | 119 | AAGCGGUCCAGCAGGAUGUUG | 410 |
| 001647 | | | | | | |
| DT- | 1175 | 1193 | AUCCUGCUGGACCGCUGUU | 120 | AACAGCGGUCCAGCAGGAUGU | 411 |
| 001648 | | | | | | |
| DT- | 1182 | 1200 | UGGACCGCUGUGGCCACAU | 121 | AUGUGGCCACAGCGGUCCAGC | 412 |
| 001649 | | | | | | |
| DT- | 1183 | 1201 | GGACCGCUGUGGCCACAUU | 122 | AAUGUGGCCACAGCGGUCCAG | 413 |
| 001650 | | | | | | |
| DT- | 1196 | 1214 | CACAUCCGCCUGGCCGACU | 123 | AGUCGGCCAGGCGGAUGUGGC | 414 |
| 001651 | | | | | | |
| DT- | 1197 | 1215 | ACAUCCGCCUGGCCGACUU | 124 | AAGUCGGCCAGGCGGAUGUGG | 415 |
| 001652 | | | | | | |
| DT- | 1198 | 1216 | CAUCCGCCUGGCCGACUUC | 125 | GAAGUCGGCCAGGCGGAUGUG | 416 |
| 001653 | | | | | | |
| DT- | 1239 | 1257 | CAGAUGGAACGGUGCGGUU | 126 | AACCGCACCGUUCCAUCUGCC | 417 |
| 001654 | | | | | | |
| DT- | 1294 | 1312 | CGAGAUCCUGCAGGCUGUU | 127 | AACAGCCUGCAGGAUCUCGGG | 418 |
| 001655 | | | | | | |
| DT- | 1320 | 1338 | GGCCUGGGACAGGCAGCUA | 128 | UAGCUGCCUGUCCCAGGCCCA | 419 |
| 001656 | | | | | | |
| DT- | 1321 | 1339 | GCCUGGGACAGGCAGCUAU | 129 | AUAGCUGCCUGUCCCAGGCCC | 420 |
| 001657 | | | | | | |
| DT- | 1373 | 1391 | UUCGCCUAUGAAAUGUUCU | 130 | AGAACAUUUCAUAGGCGAAUA | 421 |
| 001658 | | | | | | |
| DT- | 1374 | 1392 | UCGCCUAUGAAAUGUUCUA | 131 | UAGAACAUUUCAUAGGCGAAU | 422 |
| 001659 | | | | | | |
| DT- | 1394 | 1412 | GGGCAGACGCCCUUCUACU | 132 | AGUAGAAGGGCGUCUGCCCAU | 423 |
| 001660 | | | | | | |
| DT- | 1399 | 1417 | GACGCCCUUCUACGCGGAU | 133 | AUCCGCGUAGAAGGGCGUCUG | 424 |
| 001661 | | | | | | |
| DT- | 1400 | 1418 | ACGCCCUUCUACGCGGAUU | 134 | AUCCGCGUAGAAGGGCGUCUG | 425 |
| 001662 | | | | | | |
| DT- | 1401 | 1419 | CGCCCUUCUACGCGGAUUU | 135 | AAAUCCGCGUAGAAGGGCGUC | 426 |
| 001663 | | | | | | |
| DT- | 1402 | 1420 | GCCCUUCUACGCGGAUUCU | 136 | AGAAUCCGCGUAGAAGGGCGU | 427 |
| 001664 | | | | | | |
| DT- | 1404 | 1422 | CCUUCUACGCGGAUUCCAU | 137 | AUGGAAUCCGCGUAGAAGGGC | 428 |
| 001665 | | | | | | |
| DT- | 1413 | 1431 | CGGAUUCCACGGCGGAGAU | 138 | AUCUCCGCCGUGGAAUCCGCG | 429 |
| 001666 | | | | | | |
| DT- | 1416 | 1434 | AUUCCACGGCGGAGACCUA | 139 | UAGGUCUCCGCCGUGGAAUCC | 430 |
| 001667 | | | | | | |
| DT- | 1436 | 1454 | GGCAAGAUCGUCCACUACA | 140 | UGUAGUGGACGAUCUUGCCAU | 431 |
| 001668 | | | | | | |
| DT- | 1437 | 1455 | GCAAGAUCGUCCACUACAA | 141 | UUGUAGUGGACGAUCUUGCCA | 432 |
| 001669 | | | | | | |
| DT- | 1438 | 1456 | CAAGAUCGUCCACUACAAG | 142 | CUUGUAGUGGACGAUCUUGCC | 433 |
| 001670 | | | | | | |
| DT- | 1441 | 1459 | GAUCGUCCACUACAAGGAU | 143 | AUCCUUGUAGUGGACGAUCUU | 434 |
| 001671 | | | | | | |
| DT- | 1444 | 1462 | CGUCCACUACAAGGAGCAU | 144 | AUGCUCCUUGUAGUGGACGAU | 435 |
| 001672 | | | | | | |
| DT- | 1447 | 1465 | CCACUACAAGGAGCACCUU | 145 | AAGGUGCUCCUUGUAGUGGAC | 436 |
| 001673 | | | | | | |
| DT- | 1450 | 1468 | CUACAAGGAGCACCUCUCU | 146 | AGAGAGGUGCUCCUUGUAGUG | 437 |
| 001674 | | | | | | |
| DT- | 1451 | 1469 | UACAAGGAGCACCUCUCUU | 147 | AAGAGAGGUGCUCCUUGUAGU | 438 |
| 001675 | | | | | | |
| DT- | 1453 | 1471 | CAAGGAGCACCUCUCUCUU | 148 | AAGAGAGAGGUGCUCCUUGUA | 439 |
| 001676 | | | | | | |
| DT- | 1570 | 1588 | CUUCCGGACACAUCCCUUA | 149 | UAAGGGAUGUGUCCGGAAGUC | 440 |
| 001677 | | | | | | |
| DT- | 1572 | 1590 | UCCGGACACAUCCCUUCUU | 150 | AAGAAGGGAUGUGUCCGGAAG | 441 |
| 001678 | | | | | | |
| DT- | 1573 | 1591 | CCGGACACAUCCCUUCUUA | 151 | UAAGAAGGGAUGUGUCCGGAA | 442 |
| 001679 | | | | | | |
| DT- | 1575 | 1593 | GGACACAUCCCUUCUUCUU | 152 | AAGAAGAAGGGAUGUGUCCGG | 443 |
| 001680 | | | | | | |
| DT- | 1576 | 1594 | GACACAUCCCUUCUUCUUU | 153 | AAAGAAGAAGGGAUGUGUCCG | 444 |
| 001681 | | | | | | |
| DT- | 1577 | 1595 | ACACAUCCCUUCUUCUUUA | 154 | UAAAGAAGAAGGGAUGUGUCC | 445 |
| 001682 | | | | | | |
| DT- | 1578 | 1596 | CACAUCCCUUCUUCUUUGU | 155 | ACAAAGAAGAAGGGAUGUGUC | 446 |
| 001683 | | | | | | |
| DT- | 1582 | 1600 | UCCCUUCUUCUUUGGCCUU | 156 | AAGGCCAAAGAAGAAGGGAUG | 447 |
| 001684 | | | | | | |
| DT- | 1583 | 1601 | CCCUUCUUCUUUGGCCUCU | 157 | AGAGGCCAAAGAAGAAGGGAU | 448 |
| 001685 | | | | | | |
| DT- | 1585 | 1603 | CUUCUUCUUUGGCCUCGAU | 158 | AUCGAGGCCAAAGAAGAAGGG | 449 |
| 001686 | | | | | | |
| DT- | 1592 | 1610 | UUUGGCCUCGACUGGGAUU | 159 | AAUCCCAGUCGAGGCCAAAGA | 450 |
| 001687 | | | | | | |
| DT- | 1596 | 1614 | GCCUCGACUGGGAUGGUCU | 160 | AGACCAUCCCAGUCGAGGCCA | 451 |
| 001688 | | | | | | |
| DT- | 1597 | 1615 | CCUCGACUGGGAUGGUCUU | 161 | AAGACCAUCCCAGUCGAGGCC | 452 |
| 001689 | | | | | | |
| DT- | 1632 | 1650 | CCUUUACACCGGAUUUCGA | 162 | UCGAAAUCCGGUGUAAAGGGU | 453 |
| 001690 | | | | | | |
| DT- | 1633 | 1651 | CUUUACACCGGAUUUCGAA | 163 | UUCGAAAUCCGGUGUAAAGGG | 454 |
| 001691 | | | | | | |
| DT- | 1634 | 1652 | UUUACACCGGAUUUCGAAU | 164 | AUUCGAAAUCCGGUGUAAAGG | 455 |
| 001692 | | | | | | |
| DT- | 1635 | 1653 | UUACACCGGAUUUCGAAGU | 165 | ACUUCGAAAUCCGGUGUAAAG | 456 |
| 001693 | | | | | | |
| DT- | 1636 | 1654 | UACACCGGAUUUCGAAGGU | 166 | ACCUUCGAAAUCCGGUGUAAA | 457 |
| 001694 | | | | | | |
| DT- | 1637 | 1655 | ACACCGGAUUUCGAAGGUU | 167 | AACCUUCGAAAUCCGGUGUAA | 458 |
| 001695 | | | | | | |
| DT- | 1641 | 1659 | CGGAUUUCGAAGGUGCCAU | 168 | AUGGCACCUUCGAAAUCCGGU | 459 |
| 001696 | | | | | | |
| DT- | 1647 | 1665 | UCGAAGGUGCCACCGACAU | 169 | AUGUCGGUGGCACCUUCGAAA | 460 |
| 001697 | | | | | | |
| DT- | 1649 | 1667 | GAAGGUGCCACCGACACAU | 170 | AUGUGUCGGUGGCACCUUCGA | 461 |
| 001698 | | | | | | |
| DT- | 1650 | 1668 | AAGGUGCCACCGACACAUU | 171 | AAUGUGUCGGUGGCACCUUCG | 462 |
| 001699 | | | | | | |
| DT- | 1653 | 1671 | GUGCCACCGACACAUGCAA | 172 | UUGCAUGUGUCGGUGGCACCU | 463 |
| 001700 | | | | | | |
| DT- | 1654 | 1672 | UGCCACCGACACAUGCAAU | 173 | AUUGCAUGUGUCGGUGGCACC | 464 |
| 001701 | | | | | | |
| DT- | 1656 | 1674 | CCACCGACACAUGCAACUU | 174 | AAGUUGCAUGUGUCGGUGGCA | 465 |
| 001702 | | | | | | |
| DT- | 1657 | 1675 | CACCGACACAUGCAACUUU | 175 | AAAGUUGCAUGUGUCGGUGGC | 466 |
| 001703 | | | | | | |
| DT- | 1660 | 1678 | CGACACAUGCAACUUCGAU | 176 | AUCGAAGUUGCAUGUGUCGGU | 467 |
| 001704 | | | | | | |
| DT- | 1661 | 1679 | GACACAUGCAACUUCGACU | 177 | AGUCGAAGUUGCAUGUGUCGG | 468 |
| 001705 | | | | | | |
| DT- | 1662 | 1680 | ACACAUGCAACUUCGACUU | 178 | AAGUCGAAGUUGCAUGUGUCG | 469 |
| 001706 | | | | | | |
| DT- | 1663 | 1681 | CACAUGCAACUUCGACUUA | 179 | UAAGUCGAAGUUGCAUGUGUC | 470 |
| 001707 | | | | | | |
| DT- | 1664 | 1682 | ACAUGCAACUUCGACUUGU | 180 | ACAAGUCGAAGUUGCAUGUGU | 471 |
| 001708 | | | | | | |
| DT- | 1666 | 1684 | AUGCAACUUCGACUUGGUU | 181 | AACCAAGUCGAAGUUGCAUGU | 472 |
| 001709 | | | | | | |
| DT- | 1669 | 1687 | CAACUUCGACUUGGUGGAU | 182 | AUCCACCAAGUCGAAGUUGCA | 473 |
| 001710 | | | | | | |
| DT- | 1672 | 1690 | CUUCGACUUGGUGGAGGAC | 183 | GUCCUCCACCAAGUCGAAGUU | 474 |
| 001711 | | | | | | |
| DT- | 1727 | 1745 | CUGUCGGACAUUCGGGAAU | 184 | AUUCCCGAAUGUCCGACAGUG | 475 |
| 001712 | | | | | | |
| DT- | 1731 | 1749 | CGGACAUUCGGGAAGGUGC | 185 | GCACCUUCCCGAAUGUCCGAC | 476 |
| 001713 | | | | | | |
| DT- | 1779 | 1797 | GCUACUCCUACUCCUGCAU | 186 | AUGCAGGAGUAGGAGUAGCCC | 477 |
| 001714 | | | | | | |
| DT- | 1780 | 1798 | CUACUCCUACUCCUGCAUU | 187 | AAUGCAGGAGUAGGAGUAGCC | 478 |
| 001715 | | | | | | |
| DT- | 1828 | 1846 | CACACCCAUGGAACUGGAU | 188 | AUCCAGUUCCAUGGGUGUGGG | 479 |
| 001716 | | | | | | |
| DT- | 1834 | 1852 | CAUGGAACUGGAGGCCGAU | 189 | AUCGGCCUCCAGUUCCAUGGG | 480 |
| 001717 | | | | | | |
| DT- | 1852 | 1870 | GCAGCUGCUUGAGCCACAU | 190 | AUGUGGCUCAAGCAGCUGCUC | 481 |
| 001718 | | | | | | |
| DT- | 1855 | 1873 | GCUGCUUGAGCCACACGUU | 191 | AACGUGUGGCUCAAGCAGCUG | 482 |
| 001719 | | | | | | |
| DT- | 1859 | 1877 | CUUGAGCCACACGUGCAAU | 192 | AUUGCACGUGUGGCUCAAGCA | 483 |
| 001720 | | | | | | |
| DT- | 1909 | 1927 | GGAUGAAACAGCUGAAGUU | 193 | AACUUCAGCUGUUUCAUCCUG | 484 |
| 001721 | | | | | | |
| DT- | 1910 | 1928 | GAUGAAACAGCUGAAGUGU | 194 | ACACUUCAGCUGUUUCAUCCU | 485 |
| 001722 | | | | | | |
| DT- | 1913 | 1931 | GAAACAGCUGAAGUGGCAU | 195 | AUGCCACUUCAGCUGUUUCAU | 486 |
| 001723 | | | | | | |
| DT- | 1915 | 1933 | AACAGCUGAAGUGGCAGUU | 196 | AACUGCCACUUCAGCUGUUUC | 487 |
| 001724 | | | | | | |
| DT- | 1919 | 1937 | GCUGAAGUGGCAGUUCCAU | 197 | AUGGAACUGCCACUUCAGCUG | 488 |
| 001725 | | | | | | |
| DT- | 2075 | 2093 | AGUCAACUACGCGAGGCAU | 198 | AUGCCUCGCGUAGUUGACUGG | 489 |
| 001726 | | | | | | |
| DT- | 2077 | 2095 | UCAACUACGCGAGGCAGAU | 199 | AUCUGCCUCGCGUAGUUGACU | 490 |
| 001727 | | | | | | |
| DT- | 2081 | 2099 | CUACGCGAGGCAGAGGCUU | 200 | AAGCCUCUGCCUCGCGUAGUU | 491 |
| 001728 | | | | | | |
| DT- | 2086 | 2104 | CGAGGCAGAGGCUCGGAAC | 201 | GUUCCGAGCCUCUGCCUCGCG | 492 |
| 001729 | | | | | | |
| DT- | 2095 | 2113 | GGCUCGGAACCGGGACCUA | 202 | UAGGUCCCGGUUCCGAGCCUC | 493 |
| 001730 | | | | | | |
| DT- | 2096 | 2114 | GCUCGGAACCGGGACCUAU | 203 | AUAGGUCCCGGUUCCGAGCCU | 494 |
| 001731 | | | | | | |
| DT- | 2098 | 2116 | UCGGAACCGGGACCUAGAU | 204 | AUCUAGGUCCCGGUUCCGAGC | 495 |
| 001732 | | | | | | |
| DT- | 2128 | 2146 | GCAGUUGCAGGAGCGGAUU | 205 | AAUCCGCUCCUGCAACUGCCG | 496 |
| 001733 | | | | | | |
| DT- | 2133 | 2151 | UGCAGGAGCGGAUGGAGUU | 206 | AACUCCAUCCGCUCCUGCAAC | 497 |
| 001734 | | | | | | |
| DT- | 2134 | 2152 | GCAGGAGCGGAUGGAGUUA | 207 | UAACUCCAUCCGCUCCUGCAA | 498 |
| 001735 | | | | | | |
| DT- | 2137 | 2155 | GGAGCGGAUGGAGUUGCUU | 208 | AAGCAACUCCAUCCGCUCCUG | 499 |
| 001736 | | | | | | |
| DT- | 2317 | 2335 | UAGGCCUGGCCUAUCGGAU | 209 | AUCCGAUAGGCCAGGCCUAGG | 500 |
| 001737 | | | | | | |
| DT- | 2324 | 2342 | GGCCUAUCGGAGGCGCUUU | 210 | AAAGCGCCUCCGAUAGGCCAG | 501 |
| 001738 | | | | | | |
| DT- | 2325 | 2343 | GCCUAUCGGAGGCGCUUUA | 211 | UAAAGCGCCUCCGAUAGGCCA | 502 |
| 001739 | | | | | | |
| DT- | 2337 | 2355 | CGCUUUCCCUGCUCCUGUU | 212 | AACAGGAGCAGGGAAAGCGCC | 503 |
| 001740 | | | | | | |
| DT- | 2338 | 2356 | GCUUUCCCUGCUCCUGUUA | 213 | UAACAGGAGCAGGGAAAGCGC | 504 |
| 001741 | | | | | | |
| DT- | 2345 | 2363 | CUGCUCCUGUUCGCCGUUA | 214 | UAACGGCGAACAGGAGCAGGG | 505 |
| 001742 | | | | | | |
| DT- | 2413 | 2431 | CGGCCAACUCACCGCAGUU | 215 | AACUGCGGUGAGUUGGCCGGC | 506 |
| 001743 | | | | | | |
| DT- | 2415 | 2433 | GCCAACUCACCGCAGUCUU | 216 | AAGACUGCGGUGAGUUGGCCG | 507 |
| 001744 | | | | | | |
| DT- | 2455 | 2473 | CGCUCCCUGAACCCUAGAA | 217 | UUCUAGGGUUCAGGGAGCGCG | 508 |
| 001745 | | | | | | |
| DT- | 2456 | 2474 | GCUCCCUGAACCCUAGAAU | 218 | AUUCUAGGGUUCAGGGAGCGC | 509 |
| 001746 | | | | | | |
| DT- | 2460 | 2478 | CCUGAACCCUAGAACUGUC | 219 | GACAGUUCUAGGGUUCAGGGA | 510 |
| 001747 | | | | | | |
| DT- | 2461 | 2479 | CUGAACCCUAGAACUGUCU | 220 | AGACAGUUCUAGGGUUCAGGG | 511 |
| 001748 | | | | | | |
| DT- | 2462 | 2480 | UGAACCCUAGAACUGUCUU | 221 | AAGACAGUUCUAGGGUUCAGG | 512 |
| 001749 | | | | | | |
| DT- | 2463 | 2481 | GAACCCUAGAACUGUCUUU | 222 | AAAGACAGUUCUAGGGUUCAG | 513 |
| 001750 | | | | | | |
| DT- | 2464 | 2482 | AACCCUAGAACUGUCUUCU | 223 | AGAAGACAGUUCUAGGGUUCA | 514 |
| 001751 | | | | | | |
| DT- | 2465 | 2483 | ACCCUAGAACUGUCUUCGA | 224 | UCGAAGACAGUUCUAGGGUUC | 515 |
| 001752 | | | | | | |
| DT- | 2466 | 2484 | CCCUAGAACUGUCUUCGAU | 225 | AUCGAAGACAGUUCUAGGGUU | 516 |
| 001753 | | | | | | |
| DT- | 2467 | 2485 | CCUAGAACUGUCUUCGACU | 226 | AGUCGAAGACAGUUCUAGGGU | 517 |
| 001754 | | | | | | |
| DT- | 2468 | 2486 | CUAGAACUGUCUUCGACUU | 227 | AAGUCGAAGACAGUUCUAGGG | 518 |
| 001755 | | | | | | |
| DT- | 2582 | 2600 | CCAGCUCCAGUCCUGUGAU | 228 | AUCACAGGACUGGAGCUGGGC | 519 |
| 001756 | | | | | | |
| DT- | 2583 | 2601 | CAGCUCCAGUCCUGUGAUU | 229 | AAUCACAGGACUGGAGCUGGG | 520 |
| 001757 | | | | | | |
| DT- | 2672 | 2690 | CUUGUAGCCGGGAAUGCUU | 230 | AAGCAUUCCCGGCUACAAGGA | 521 |
| 001758 | | | | | | |
| DT- | 2676 | 2694 | UAGCCGGGAAUGCUGCUGU | 231 | ACAGCAGCAUUCCCGGCUACA | 522 |
| 001759 | | | | | | |
| DT- | 2769 | 2787 | UUCUUUCGGCCAGGCUGAA | 232 | UUCAGCCUGGCCGAAAGAAAG | 523 |
| 001760 | | | | | | |
| DT- | 2783 | 2801 | CUGAGGCCCUGACGUGGAU | 233 | AUCCACGUCAGGGCCUCAGCC | 524 |
| 001761 | | | | | | |
| DT- | 2784 | 2802 | UGAGGCCCUGACGUGGAUU | 234 | AAUCCACGUCAGGGCCUCAGC | 525 |
| 001762 | | | | | | |
| DT- | 2789 | 2807 | CCCUGACGUGGAUGGGCAA | 235 | UUGCCCAUCCACGUCAGGGCC | 526 |
| 001763 | | | | | | |
| DT- | 2790 | 2808 | CCUGACGUGGAUGGGCAAA | 236 | UUUGCCCAUCCACGUCAGGGC | 527 |
| 001764 | | | | | | |
| DT- | 2791 | 2809 | CUGACGUGGAUGGGCAAAU | 237 | AUUUGCCCAUCCACGUCAGGG | 528 |
| 001765 | | | | | | |
| DT- | 2902 | 2920 | UGUGCAUGACGCCCUGCUC | 238 | GAGCAGGGCGUCAUGCACAAG | 529 |
| 001766 | | | | | | |
| DT- | 2903 | 2921 | GUGCAUGACGCCCUGCUCU | 239 | AGAGCAGGGCGUCAUGCACAA | 530 |
| 001767 | | | | | | |
| DT- | 2949 | 2967 | GCUUACUCGGGAAAUUUGA | 240 | UCAAAUUUCCCGAGUAAGCAG | 531 |
| 001768 | | | | | | |
| DT- | 2950 | 2968 | CUUACUCGGGAAAUUUGCU | 241 | AGCAAAUUUCCCGAGUAAGCA | 532 |
| 001769 | | | | | | |
| DT- | 2951 | 2969 | UUACUCGGGAAAUUUGCUU | 242 | AAGCAAAUUUCCCGAGUAAGC | 533 |
| 001770 | | | | | | |
| DT- | 2952 | 2970 | UACUCGGGAAAUUUGCUUU | 243 | AAAGCAAAUUUCCCGAGUAAG | 534 |
| 001771 | | | | | | |
| DT- | 2953 | 2971 | ACUCGGGAAAUUUGCUUUU | 244 | AAAAGCAAAUUUCCCGAGUAA | 535 |
| 001772 | | | | | | |
| DT- | 2954 | 2972 | CUCGGGAAAUUUGCUUUUA | 245 | UAAAAGCAAAUUUCCCGAGUA | 536 |
| 001773 | | | | | | |
| DT- | 2955 | 2973 | UCGGGAAAUUUGCUUUUGU | 246 | UCAAAAGCAAAUUUCCCGAGU | 537 |
| 001774 | | | | | | |
| DT- | 2958 | 2976 | GGAAAUUUGCUUUUGCCAA | 247 | UUGGCAAAAGCAAAUUUCCCG | 538 |
| 001775 | | | | | | |
| DT- | 2959 | 2977 | GAAAUUUGCUUUUGCCAAA | 248 | UUUGGCAAAAGCAAAUUUCCC | 539 |
| 001776 | | | | | | |
| DT- | 2960 | 2978 | AAAUUUGCUUUUGCCAAAU | 249 | AUUUGGCAAAAGCAAAUUUCC | 540 |
| 001777 | | | | | | |
| DT- | 2966 | 2984 | GCUUUUGCCAAACCCGCUU | 250 | AAGCGGGUUUGGCAAAAGCAA | 541 |
| 001778 | | | | | | |
| DT- | 3049 | 3067 | GCUUCGGCGGUUUGGAUAU | 251 | AUAUCCAAACCGCCGAAGCGG | 542 |
| 001779 | | | | | | |
| DT- | 3050 | 3068 | CUUCGGCGGUUUGGAUAUU | 252 | AAUAUCCAAACCGCCGAAGCG | 543 |
| 001780 | | | | | | |
| DT- | 3051 | 3069 | UUCGGCGGUUUGGAUAUUU | 253 | AAAUAUCCAAACCGCCGAAGC | 544 |
| 001781 | | | | | | |
| DT- | 3052 | 3070 | UCGGCGGUUUGGAUAUUUA | 254 | UAAAUAUCCAAACCGCCGAAG | 545 |
| 001782 | | | | | | |
| DT- | 3053 | 3071 | CGGCGGUUUGGAUAUUUAU | 255 | AUAAAUAUCCAAACCGCCGAA | 546 |
| 001783 | | | | | | |
| DT- | 3054 | 3072 | GGCGGUUUGGAUAUUUAUU | 256 | AAUAAAUAUCCAAACCGCCGA | 547 |
| 001784 | | | | | | |
| DT- | 3055 | 3073 | GCGGUUUGGAUAUUUAUUU | 257 | AAAUAAAUAUCCAAACCGCCG | 548 |
| 001785 | | | | | | |
| DT- | 3056 | 3074 | CGGUUUGGAUAUUUAUUGA | 258 | UCAAUAAAUAUCCAAACCGCC | 549 |
| 001786 | | | | | | |
| DT- | 3057 | 3075 | GGUUUGGAUAUUUAUUGAU | 259 | AUCAAUAAAUAUCCAAACCGC | 550 |
| 001787 | | | | | | |
| DT- | 3058 | 3076 | GUUUGGAUAUUUAUUGACU | 260 | AGUCAAUAAAUAUCCAAACCG | 551 |
| 001788 | | | | | | |
| DT- | 3059 | 3077 | UUUGGAUAUUUAUUGACCU | 261 | AGGUCAAUAAAUAUCCAAACC | 552 |
| 001789 | | | | | | |
| DT- | 3060 | 3078 | UUGGAUAUUUAUUGACCUU | 262 | AAGGUCAAUAAAUAUCCAAAC | 553 |
| 001790 | | | | | | |
| DT- | 3061 | 3079 | UGGAUAUUUAUUGACCUCU | 263 | AGAGGUCAAUAAAUAUCCAAA | 554 |
| 001791 | | | | | | |
| DT- | 3062 | 3080 | GGAUAUUUAUUGACCUCGU | 264 | ACGAGGUCAAUAAAUAUCCAA | 555 |
| 001792 | | | | | | |
| DT- | 3063 | 3081 | GAUAUUUAUUGACCUCGUA | 265 | UACGAGGUCAAUAAAUAUCCA | 556 |
| 001793 | | | | | | |
| DT- | 3064 | 3082 | AUAUUUAUUGACCUCGUCU | 266 | AGACGAGGUCAAUAAAUAUCC | 557 |
| 001794 | | | | | | |
| DT- | 3067 | 3085 | UUUAUUGACCUCGUCCUCA | 267 | UGAGGACGAGGUCAAUAAAUA | 558 |
| 001795 | | | | | | |
| DT- | 3070 | 3088 | AUUGACCUCGUCCUCCGAU | 268 | AUCGGAGGACGAGGUCAAUAA | 559 |
| 001796 | | | | | | |
| DT- | 3077 | 3095 | UCGUCCUCCGACUCGCUGA | 269 | UCAGCGAGUCGGAGGACGAGG | 560 |
| 001797 | | | | | | |
| DT- | 3080 | 3098 | UCCUCCGACUCGCUGACAU | 270 | AUGUCAGCGAGUCGGAGGACG | 561 |
| 001798 | | | | | | |
| DT- | 3119 | 3137 | CCCAAUCCACGUUUUGGAU | 271 | AUCCAAAACGUGGAUUGGGUG | 562 |
| 001799 | | | | | | |
| DT- | 3120 | 3138 | CCAAUCCACGUUUUGGAUU | 272 | AAUCCAAAACGUGGAUUGGGU | 563 |
| 001800 | | | | | | |
| DT- | 3121 | 3139 | CAAUCCACGUUUUGGAUGA | 273 | UCAUCCAAAACGUGGAUUGGG | 564 |
| 001801 | | | | | | |
| DT- | 3122 | 3140 | AAUCCACGUUUUGGAUGCA | 274 | UGCAUCCAAAACGUGGAUUGG | 565 |
| 001802 | | | | | | |
| DT- | 3123 | 3141 | AUCCACGUUUUGGAUGCAU | 275 | AUGCAUCCAAAACGUGGAUUG | 566 |
| 001803 | | | | | | |
| DT- | 3125 | 3143 | CCACGUUUUGGAUGCACUU | 276 | AAGUGCAUCCAAAACGUGGAU | 567 |
| 001804 | | | | | | |
| DT- | 3126 | 3144 | CACGUUUUGGAUGCACUGA | 277 | UCAGUGCAUCCAAAACGUGGA | 568 |
| 001805 | | | | | | |
| DT- | 3127 | 3145 | ACGUUUUGGAUGCACUGAU | 278 | AUCAGUGCAUCCAAAACGUGG | 569 |
| 001806 | | | | | | |
| DT- | 3128 | 3146 | CGUUUUGGAUGCACUGAGA | 279 | UCUCAGUGCAUCCAAAACGUG | 570 |
| 001807 | | | | | | |
| DT- | 3129 | 3147 | GUUUUGGAUGCACUGAGAU | 280 | AUCUCAGUGCAUCCAAAACGU | 571 |
| 001808 | | | | | | |
| DT- | 3130 | 3148 | UUUUGGAUGCACUGAGACU | 281 | AGUCUCAGUGCAUCCAAAACG | 572 |
| 001809 | | | | | | |
| DT- | 3157 | 3175 | CCUCGGUAUUUAUUGUCUA | 282 | UAGACAAUAAAUACCGAGGAA | 573 |
| 000841 | | | | | | |
| DT- | 3197 | 3215 | CCCGACCCUCGCGAAUAAA | 283 | UUUAUUCGCGAGGGUCGGGUG | 574 |
| 001810 | | | | | | |
| DT- | 3198 | 3216 | CCGACCCUCGCGAAUAAAA | 284 | UUUUAUUCGCGAGGGUCGGGU | 575 |
| 001811 | | | | | | |
| DT- | 3199 | 3217 | CGACCCUCGCGAAUAAAAU | 285 | AUUUUAUUCGCGAGGGUCGGG | 576 |
| 001812 | | | | | | |
| DT- | 3200 | 3218 | GACCCUCGCGAAUAAAAGU | 286 | ACUUUUAUUCGCGAGGGUCGG | 577 |
| 001813 | | | | | | |
| DT- | 3201 | 3219 | ACCCUCGCGAAUAAAAGGU | 287 | ACCUUUUAUUCGCGAGGGUCG | 578 |
| 001814 | | | | | | |
| DT- | 3202 | 3220 | CCCUCGCGAAUAAAAGGCU | 288 | AGCCUUUUAUUCGCGAGGGUC | 579 |
| 001815 | | | | | | |
| DT- | 3205 | 3223 | UCGCGAAUAAAAGGCCCUA | 289 | UAGGGCCUUUUAUUCGCGAGG | 580 |
| 001816 | | | | | | |
| DT- | 3206 | 3224 | CGCGAAUAAAAGGCCCUCA | 290 | UGAGGGCCUUUUAUUCGCGAG | 581 |
| 001817 | | | | | | |
| DT- | 3207 | 3225 | GCGAAUAAAAGGCCCUCCA | 291 | UGGAGGGCCUUUUAUUCGCGA | 582 |
| 001818 | | | | | | |
| DT- | 3208 | 3226 | CGAAUAAAAGGCCCUCCAU | 292 | AUGGAGGGCCUUUUAUUCGCG | 583 |
| 001819 | | | | | | |
| DT- | 3209 | 3227 | GAAUAAAAGGCCCUCCAUU | 293 | AAUGGAGGGCCUUUUAUUCGC | 584 |
| 001820 | | | | | | |

In Table 1, each code (letter, e.g., A, G, C, and U) represents a single ribonucleotide with a nucleobase. For example, “A” represent a unmodified ribonucleotide with adenine; “G” represents a unmodified ribonucleotide with guanine; “C” represents a C: unmodified ribonucleotide with cytosine; and “U” represents a unmodified ribonucleotide with uracil.

Exemplary siRNAs described herein may have the pattern of modified sugar moieties and internucleoside linkages. The antisense strands are modified such that, counting from the 5′ terminus of the antisense strand, nucleotides 1, 3, 5, 7, 9, 11, 13, 15, 17, 19, 20, and 21 are 2′-O-methyl nucleotides, nucleotides 2, 4, 6, 8, 10, 12, 14, 16, and 18 are 2′-fluoro nucleotides, the first two internucleoside linkages at the 5′ terminus and the last two internucleoside linkages at the 3′ terminus are phosphorothioate internucleoside linkages, and each other antinucleoside linkage is a phosphodiester internucleoside linkage. The sense strands are modified such that, counting from the 5′ terminus of the sense strand, nucleotides 1, 3, 5, 7, 9, 11, 13, 15, 17, and 19 are 2′-fluoro nucleotides and nucleotides 2, 4, 6, 8, 10, 12, 14, 16, and 18 are 2′-O-methyl nucleotides, the first two internucleoside linkages at the 5′ terminus and the last two internucleoside linkages at the 3′ terminus are phosphorothioate internucleoside linkages, and each other internucleotide is a phosphodiester internucleoside linkage.

TABLE 2
Unconjugated siRNAs targeting DMPK (modified sequences)

| siRNA | | SEQ | | SEQ |
|---|---|---|---|---|
| compound | Sense Strand | ID | Antisense Strand | ID |
| ID | modified sequence (5′→3′) | NO | modified sequence (5′→3′) | NO: |
| DT- | A007p001G004p001G007p000C0 | 585 | X033A004p001U007p001C004p00 | 876 |
| 001531 | 04p000U007p000U004p000A007 | | 0G007p000G004p000A007p000C0 | |
| | p000A004p000G007p000G004p0 | | 04p000C007p000U004p000C007p | |
| | 00A007p000G004p000G007p000 | | 000C004p000U007p000U004p000 | |
| | U004p000C007p000C004p000G0 | | A007p000A004p000G007p000C00 | |
| | 07p001A004p001U007 | | 4p000C007p000U004p001C004p0 | |
| | | | 01A004 | |
| DT- | G007p001G004p001C007p000U0 | 586 | X033A004p001G007p001U004p00 | 877 |
| 001532 | 04p000U007p000A004p000A007 | | 0C007p000G004p000G007p000A0 | |
| | p000G004p000G007p000A004p0 | | 04p000C007p000C004p000U007p | |
| | 00G007p000G004p000U007p000 | | 000C004p000C007p000U004p000 | |
| | C004p000C007p000G004p000A0 | | U007p000A004p000A007p000G00 | |
| | 07p001C004p001U007 | | 4p000C007p000C004p001U004p0 | |
| | | | 01C004 | |
| DT- | G007p001C004p001U007p000U0 | 587 | X033A004p001A007p001G004p00 | 878 |
| 001533 | 04p000A007p000A004p000G007 | | 0U007p000C004p000G007p000G0 | |
| | p000G004p000A007p000G004p0 | | 04p000A007p000C004p000C007p | |
| | 00G007p000U004p000C007p000 | | 0000004p000C007p000C004p000 | |
| | C004p000G007p000A004p000C0 | | U007p000U004p000A007p000A00 | |
| | 07p001U004p001U007 | | 4p000G007p000C004p001C004p0 | |
| | | | 010004 | |
| DT- | A007p001A004p001G007p000U0 | 588 | X0330004p001C007p001U004p00 | 879 |
| 001534 | 04p000G007p000G004p000G007 | | 0U007p000C004p000A007p000G0 | |
| | p000A004p000C007p000A004p0 | | 04p000C007p000A004p000U007p | |
| | 00U007p000G004p000C007p000 | | 000G004p000U007p000C004p000 | |
| | U004p000G007p000A004p000A0 | | C007p000C004p000A007p000C00 | |
| | 07p001G004p001A007 | | 4p000U007p000U004p001G004p0 | |
| | | | 01U004 | |
| DT- | A007p001G004p001U007p000G0 | 589 | X033A004p001U007p001C004p00 | 880 |
| 001535 | 04p000G007p000G004p000A007 | | 0U007p000U004p000C007p000A0 | |
| | p000C004p000A007p000U004p0 | | 04p000G007p000C004p000A007p | |
| | 00G007p000C004p000U007p000 | | 0000004p000G007p000U004p000 | |
| | G004p000A007p000A004p000G0 | | C007p000C004p000C007p000A00 | |
| | 07p001A004p001U007 | | 4p000C007p000U004p001U004p0 | |
| | | | 01G004 | |
| DT- | G007p001G004p001C007p000G0 | 590 | X033A004p001G007p001A004p00 | 881 |
| 001536 | 04p000A007p000G004p000G007 | | 0A007p000G004p000C007p000A0 | |
| | p000U004p000G007p000U004p0 | | 04p000C007p000G004p000A007p | |
| | 00C007p000G004p000U007p000 | | 000C004p000A007p000C004p000 | |
| | G004p000C007p000U004p000U0 | | C007p000U004p000C007p000G00 | |
| | 07p001C004p001U007 | | 4p000C007p000C004p001C004p0 | |
| | | | 01C004 | |
| DT- | G007p001C004p001C007p000U0 | 591 | X033A004p001G007p001U004p00 | 882 |
| 001537 | 04p000U007p000C004p000C007 | | 0U007p000C004p000U007p000C0 | |
| | p000A004p000G007p000G004p0 | | 04p000A007p000U004p000C007p | |
| | 00A007p000U004p000G007p000 | | 000C004p000U007p000G004p000 | |
| | A004p000G007p000A004p000A0 | | G007p000A004p000A007p000G00 | |
| | 07p001C004p001U007 | | 4p000G007p000C004p001G004p0 | |
| | | | 01A004 | |
| DT- | C007p001C004p001U007p000U0 | 592 | X0330004p001A007p001G004p00 | 883 |
| 001538 | 04p000C007p000C004p000A007 | | 0U007p000U004p000C007p000U0 | |
| | p000G004p000G007p000A004p0 | | 04p000C007p000A004p000U007p | |
| | 00U007p000G004p000A007p000 | | 000C004p000C007p000U004p000 | |
| | G004p000A007p000A004p000C0 | | G007p000G004p000A007p000A00 | |
| | 07p001U004p001A007 | | 4p000G007p000G004p001C004p0 | |
| | | | 01G004 | |
| DT- | C007p001U004p001U007p000C0 | 593 | X033A004p001U007p001A004p00 | 884 |
| 001539 | 04p000C007p000A004p000G007 | | 0G007p000U004p000U007p000C0 | |
| | p000G004p000A007p000U004p0 | | 04p000U007p000C004p000A007p | |
| | 00G007p000A004p000G007p000 | | 0000004p000C007p000C004p000 | |
| | A004p000A007p000C004p000U0 | | U007p000G004p000G007p000A00 | |
| | 07p001A004p001U007 | | 4p000A007p000G004p001G004p0 | |
| | | | 01C004 | |
| DT- | C007p001C004p001A007p000G0 | 594 | X033U004p001A007p001G004p00 | 885 |
| 001540 | 04p000G007p000A004p000U007 | | 0G007p000U004p000A007p000G0 | |
| | p000G004p000A007p000G004p0 | | 04p000U007p000U004p000C007p | |
| | 00A007p000A004p000C007p000 | | 0000004p000C007p000A004p000 | |
| | 0004p000A007p000C004p000C0 | | U007p000C004p000C007p000U00 | |
| | 07p001U004p001A007 | | 4p000G007p000G004p001A004p0 | |
| | | | 01A004 | |
| DT- | C007p001A004p001G007p000G0 | 595 | X033A004p001C007p001A004p00 | 886 |
| 001541 | 04p000A007p000U004p000G007 | | 0G007p000G004p000U007p000A0 | |
| | p000A004p000G007p000A004p0 | | 04p000G007p000U004p000U007p | |
| | 00A007p000C004p000U007p000 | | 000C004p000U007p000C004p000 | |
| | A004p000C007p000C004p000U0 | | A007p000U004p000C007p000C00 | |
| | 07p001G004p001U007 | | 4p000U007p000G004p001G004p0 | |
| | | | 01A004 | |
| DT- | A007p001G004p001G007p000A0 | 596 | X0330004p001A007p001C004p00 | 887 |
| 001542 | 04p000U007p000G004p000A007 | | 0A007p000G004p000G007p000U0 | |
| | p000G004p000A007p000A004p0 | | 04p000A007p000G004p000U007p | |
| | 00C007p000U004p000A007p000 | | 0000004p000C007p000U004p000 | |
| | C004p000C007p000U004p000G0 | | C007p000A004p000U007p000C00 | |
| | 07p001U004p001A007 | | 4p000C007p000U004p001G004p0 | |
| | | | 01G004 | |
| DT- | G007p001G004p001A007p000U0 | 597 | X033A004p001U007p001A004p00 | 888 |
| 001543 | 04p000G007p000A004p000G007 | | 0C007p000A004p000G007p000G0 | |
| | p000A004p000A007p000C004p0 | | 04p000U007p000A004p000G007p | |
| | 00U007p000A004p000C007p000 | | 0000004p000U007p000C004p000 | |
| | C004p000U007p000G004p000U0 | | U007p000C004p000A007p000U00 | |
| | 07p001A004p001U007 | | 4p000C007p000C004p001U004p0 | |
| | | | 01G004 | |
| DT- | A007p001C004p001U007p000A0 | 598 | X033A004p001U007p001G004p00 | 889 |
| 001544 | 04p000C007p000C004p000U007 | | 0A007p000C004p000C007p000A0 | |
| | p000G004p000U007p000A004p0 | | 04p000G007p000G004p000U007p | |
| | 00C007p000C004p000U007p000 | | 000A004p000C007p000A004p000 | |
| | G004p000G007p000U004p000C0 | | G007p000G004p000U007p000A00 | |
| | 07p001A004p001U007 | | 4p000G007p000U004p001U004p0 | |
| | | | 01C004 | |
| DT- | C007p001U004p001A007p000C0 | 599 | X033A004p001A007p001U004p00 | 890 |
| 001545 | 04p000C007p000U004p000G007 | | 0G007p000A004p000C007p000C0 | |
| | p000U004p000A007p000C004p0 | | 04p000A007p000G004p000G007p | |
| | 00C007p000U004p000G007p000 | | 0000004p000A007p000C004p000 | |
| | G004p000U007p000C004p000A0 | | A007p000G004p000G007p000U00 | |
| | 07p001U004p001U007 | | 4p000A007p000G004p001U004p0 | |
| | | | 01U004 | |
| DT- | U007p001A004p001C007p000C0 | 600 | X033A004p001C007p001A004p00 | 891 |
| 001546 | 04p000U007p000G004p000U007 | | 0U007p000G004p000A007p000C0 | |
| | p000A004p000C007p000C004p0 | | 04p000C007p000A004p000G007p | |
| | 00U007p000G004p000G007p000 | | 000G004p000U007p000A004p000 | |
| | U004p000C007p000A004p000U0 | | C007p000A004p000G007p000G00 | |
| | 07p001G004p001U007 | | 4p000U007p000A004p001G004p0 | |
| | | | 01U004 | |
| DT- | A007p001C004p001C007p000U0 | 601 | X0330004p001C007p001C004p00 | 892 |
| 001547 | 04p000G007p000U004p000A007 | | 0A007p000U004p000G007p000A0 | |
| | p000C004p000C007p000U004p0 | | 04p000C007p000C004p000A007p | |
| | 00G007p000G004p000U007p000 | | 000G004p000G007p000U004p000 | |
| | C004p000A007p000U004p000G0 | | A007p000C004p000A007p000G00 | |
| | 07p001G004p001A007 | | 4p000G007p000U004p001A004p0 | |
| | | | 01G004 | |
| DT- | G007p001C004p001U007p000G0 | 602 | X033A004p001U007p001C004p00 | 893 |
| 001548 | 04p000A007p000G004p000C007 | | 0C007p000C004p000C007p000A0 | |
| | p000A004p000A007p000G004p0 | | 04p000A007p000A004p000C007p | |
| | 00U007p000U004p000U007p000 | | 0000004p000U007p000G004p000 | |
| | G004p000G007p000G004p000G0 | | C007p000U004p000C007p000A00 | |
| | 07p001A004p001U007 | | 4p000G007p000C004p001A004p0 | |
| | | | 01G004 | |
| DT- | G007p001C004p001A007p000A0 | 603 | X033A004p001U007p001C004p00 | 894 |
| 001549 | 04p000G007p000U004p000U007 | | 0C007p000G004p000C007p000U0 | |
| | p000U004p000G007p000G004p0 | | 04p000C007p000C004p000C007p | |
| | 00G007p000G004p000A007p000 | | 000C004p000A007p000A004p000 | |
| | G004p000C007p000G004p000G0 | | A007p000C004p000U007p000U00 | |
| | 07p001A004p001U007 | | 4p000G007p000C004p001U004p0 | |
| | | | 01C004 | |
| DT- | G007p001A004p001G007p000A0 | 604 | X033A004p001U007p001A004p00 | 895 |
| 001550 | 04p000U007p000U004p000G007 | | 0U007p000G004p000G007p000C0 | |
| | p000U004p000C007p000A004p0 | | 04p000C007p000A004p000U007p | |
| | 00U007p000G004p000G007p000 | | 000G004p000A007p000C004p000 | |
| | C004p000C007p000A004p000U0 | | A007p000A004p000U007p000C00 | |
| | 07p001A004p001U007 | | 4p000U007p000C004p001C004p0 | |
| | | | 01G004 | |
| DT- | A007p001G004p001A007p000U0 | 605 | X033U004p001C007p001U004p00 | 896 |
| 001551 | 04p000U007p000G004p000U007 | | 0A007p000U004p000G007p000G0 | |
| | p000C004p000A007p000U004p0 | | 04p000C007p000C004p000A007p | |
| | 00G007p000G004p000C007p000 | | 000U004p000G007p000A004p000 | |
| | C004p000A007p000U004p000A0 | | C007p000A004p000A007p000U00 | |
| | 07p001G004p001A007 | | 4p000C007p000U004p001C004p0 | |
| | | | 01C004 | |
| DT- | G007p001G004p001C007p000U0 | 606 | X033U004p001G007p001U004p00 | 897 |
| 001552 | 04p000A007p000C004p000G007 | | 0C007p000C004p000C007p000U0 | |
| | p000U004p000G007p000C004p0 | | 04p000G007p000U004p000G007p | |
| | 00A007p000C004p000A007p000 | | 000C004p000A007p000C004p000 | |
| | G004p000G007p000G004p000A0 | | G007p000U004p000A007p000G00 | |
| | 07p001C004p001A007 | | 4p000C007p000C004p001A004p0 | |
| | | | 01A004 | |
| DT- | G007p001C004p001U007p000A0 | 607 | X033A004p001U007p001G004p00 | 898 |
| 001553 | 04p000C007p000G004p000U007 | | 0U007p000C004p000C007p000C0 | |
| | p000G004p000C007p000A004p0 | | 04p000U007p000G004p000U007p | |
| | 00C007p000A004p000G007p000 | | 000G004p000C007p000A004p000 | |
| | G004p000G007p000A004p000C0 | | C007p000G004p000U007p000A00 | |
| | 07p001A004p001U007 | | 4p000G007p000C004p001C004p0 | |
| | | | 01A004 | |
| DT- | C007p001U004p001A007p000C0 | 608 | X033A004p001A007p001U004p00 | 899 |
| 001554 | 04p000G007p000U004p000G007 | | 0G007p000U004p000C007p000C0 | |
| | p000C004p000A007p000C004p0 | | 04p000C007p000U004p000G007p | |
| | 00A007p000G004p000G007p000 | | 0000004p000G007p000C004p000 | |
| | G004p000A007p000C004p000A0 | | A007p000C004p000G007p000U00 | |
| | 07p001U004p001U007 | | 4p000A007p000G004p001C004p0 | |
| | | | 01C004 | |
| DT- | U007p001A004p001C007p000G0 | 609 | X0330004p001G007p001A004p00 | 900 |
| 001555 | 04p000U007p000G004p000C007 | | 0U007p000G004p000U007p000C0 | |
| | p000A004p000C007p000A004p0 | | 04p000C007p000C004p000U007p | |
| | 00G007p000G004p000G007p000 | | 000G004p000U007p000G004p000 | |
| | A004p000C007p000A004p000U0 | | C007p000A004p000C007p000G00 | |
| | 07p001C004p001A007 | | 4p000U007p000A004p001G004p0 | |
| | | | 01C004 | |
| DT- | A007p001C004p001G007p000U0 | 610 | X0330004p001U007p001G004p00 | 901 |
| 001556 | 04p000G007p000C004p000A007 | | 0A007p000U004p000G007p000U0 | |
| | p000C004p000A007p000G004p0 | | 04p000C007p000C004p000C007p | |
| | 00G007p000G004p000A007p000 | | 0000004p000G007p000U004p000 | |
| | C004p000A007p000U004p000C0 | | G007p000C004p000A007p000C00 | |
| | 07p001A004p001A007 | | 4p000G007p000U004p001A004p0 | |
| | | | 01G004 | |
| DT- | C007p001G004p001U007p000G0 | 611 | X0330004p001U007p001U004p00 | 902 |
| 001557 | 04p000C007p000A004p000C007 | | 0G007p000A004p000U007p000G0 | |
| | p000A004p000G007p000G004p0 | | 04p000U007p000C004p000C007p | |
| | 00G007p000A004p000C007p000 | | 000C004p000U007p000G004p000 | |
| | A004p000U007p000C004p000A0 | | U007p000G004p000C007p000A00 | |
| | 07p001A004p001A007 | | 4p000C007p000G004p001U004p0 | |
| | | | 01A004 | |
| DT- | G007p001U004p001G007p000C0 | 612 | X033A004p001U007p001U004p00 | 903 |
| 001558 | 04p000A007p000C004p000A007 | | 0U007p000G004p000A007p000U0 | |
| | p000G004p000G007p000G004p0 | | 04p000G007p000U004p000C007p | |
| | 00A007p000C004p000A007p000 | | 000C004p000C007p000U004p000 | |
| | U004p000C007p000A004p000A0 | | G007p000U004p000G007p000C00 | |
| | 07p001A004p001U007 | | 4p000A007p000C004p001G004p0 | |
| | | | 01U004 | |
| DT- | U007p001G004p001C007p000A0 | 613 | X033A004p001G007p001U004p00 | 904 |
| 001559 | 04p000C007p000A004p000G007 | | 0U007p000U004p000G007p000A0 | |
| | p000G004p000G007p000A004p0 | | 04p000U007p000G004p000U007p | |
| | 00C007p000A004p000U007p000 | | 000C004p000C007p000C004p000 | |
| | C004p000A007p000A004p000A0 | | U007p000G004p000U007p000G00 | |
| | 07p001C004p001U007 | | 4p000C007p000A004p001C004p0 | |
| | | | 01G004 | |
| DT- | G007p001G004p001C007p000U0 | 614 | X033A004p001U007p001C004p00 | 905 |
| 001560 | 04p000C007p000A004p000C007 | | 0A007p000C004p000C007p000A0 | |
| | p000U004p000G007p000C004p0 | | 04p000U007p000G004p000G007p | |
| | 00C007p000A004p000U007p000 | | 000C004p000A007p000G004p000 | |
| | G004p000G007p000U004p000G0 | | U007p000G004p000A007p000G00 | |
| | 07p001A004p001U007 | | 4p000C007p000C004p001C004p0 | |
| | | | 01G004 | |
| DT- | G007p001C004p001U007p000C0 | 615 | X033A004p001C007p001U004p00 | 906 |
| 001561 | 04p000A007p000C004p000U007 | | 0C007p000A004p000C007p000C0 | |
| | p000G004p000C007p000C004p0 | | 04p000A007p000U004p000G007p | |
| | 00A007p000U004p000G007p000 | | 000G004p000C007p000A004p000 | |
| | G004p000U007p000G004p000A0 | | G007p000U004p000G007p000A00 | |
| | 07p001G004p001U007 | | 4p000G007p000C004p001C004p0 | |
| | | | 01C004 | |
| DT- | C007p001U004p001C007p000A0 | 616 | X033A004p001G007p001C004p00 | 907 |
| 001562 | 04p000C007p000U004p000G007 | | 0U007p000C004p000A007p000C0 | |
| | p000C004p000C007p000A004p0 | | 04p000C007p000A004p000U007p | |
| | 00U007p000G004p000G007p000 | | 000G004p000G007p000C004p000 | |
| | 0004p000G007p000A004p000G0 | | A007p000G004p000U007p000G00 | |
| | 07p001C004p001U007 | | 4p000A007p000G004p001C004p0 | |
| | | | 01C004 | |
| DT- | U007p001C004p001A007p000C0 | 617 | X033A004p001C007p001G004p00 | 908 |
| 001563 | 04p000U007p000G004p000C007 | | 0C007p000U004p000C007p000A0 | |
| | p000C004p000A007p000U004p0 | | 04p000C007p000C004p000A007p | |
| | 00G007p000G004p000U007p000 | | 0000004p000G007p000G004p000 | |
| | G004p000A007p000G004p000C0 | | C007p000A004p000G007p000U00 | |
| | 07p001G004p001U007 | | 4p000G007p000A004p001G004p0 | |
| | | | 01C004 | |
| DT- | C007p001U004p001C007p000A0 | 618 | X0330004p001C007p001A004p00 | 909 |
| 001564 | 04p000C007p000C004p000C007 | | 0G007p000G004p000C007p000U0 | |
| | p000G004p000G007p000C004p0 | | 04p000C007p000U004p000G007p | |
| | 00A007p000G004p000A007p000 | | 000C004p000C007p000G004p000 | |
| | G004p000C007p000C004p000U0 | | G007p000G004p000U007p000G00 | |
| | 07p001G004p001A007 | | 4p000A007p000G004p001C004p0 | |
| | | | 01A004 | |
| DT- | U007p001C004p001A007p000C0 | 619 | X033A004p001U007p001C004p00 | 910 |
| 001565 | 04p000C007p000C004p000G007 | | 0A007p000G004p000G007p000C0 | |
| | p000G004p000C007p000A004p0 | | 04p000U007p000C004p000U007p | |
| | 00G007p000A004p000G007p000 | | 000G004p000C007p000C004p000 | |
| | C004p000C007p000U004p000G0 | | G007p000G004p000G007p000U00 | |
| | 07p001A004p001U007 | | 4p000G007p000A004p001G004p0 | |
| | | | 01C004 | |
| DT- | C007p001A004p001C007p000G0 | 620 | X033A004p001U007p001G004p00 | 911 |
| 001566 | 04p000G007p000A004p000U007 | | 0G007p000G004p000A007p000A0 | |
| | p000C004p000C007p000A004p0 | | 04p000G007p000G004p000U007p | |
| | 00C007p000C004p000U007p000 | | 000G004p000G007p000A004p000 | |
| | 0004p000C007p000C004p000C0 | | U007p000C004p000C007p000G00 | |
| | 07p001A004p001U007 | | 4p000U007p000G004p001G004p0 | |
| | | | 01C004 | |
| DT- | A007p001C004p001G007p000G0 | 621 | X033G004p001A007p001U004p00 | 912 |
| 001567 | 04p000A007p000U004p000C007 | | 0G007p000G004p000G007p000A0 | |
| | p000C004p000A007p000C004p0 | | 04p000A007p000G004p000G007p | |
| | 00C007p000U004p000U007p000 | | 0000004p000G007p000G004p000 | |
| | C004p000C007p000C004p000A0 | | A007p000U004p000C007p000C00 | |
| | 07p001U004p001C007 | | 4p000G007p000U004p001G004p0 | |
| | | | 01G004 | |
| DT- | C007p001G004p001G007p000A0 | 622 | X033A004p001G007p001A004p00 | 913 |
| 001568 | 04p000U007p000C004p000C007 | | 0U007p000G004p000G007p000G0 | |
| | p000A004p000C007p000C004p0 | | 04p000A007p000A004p000G007p | |
| | 00U007p000U004p000C007p000 | | 000G004p000U007p000G004p000 | |
| | C004p000C007p000A004p000U0 | | G007p000A004p000U007p000C00 | |
| | 07p001C004p001U007 | | 4p000C007p000G004p001U004p0 | |
| | | | 01G004 | |
| DT- | G007p001G004p001A007p000U0 | 623 | X0330004p001A007p001G004p00 | 914 |
| 001569 | 04p000C007p000C004p000A007 | | 0A007p000U004p000G007p000G0 | |
| | p000C004p000C007p000U004p0 | | 04p000G007p000A004p000A007p | |
| | 00U007p000C004p000C007p000 | | 000G004p000G007p000U004p000 | |
| | C004p000A007p000U004p000C0 | | G007p000G004p000A007p000U00 | |
| | 07p001U004p001A007 | | 4p000C007p000C004p001G004p0 | |
| | | | 01U004 | |
| DT- | G007p001A004p001U007p000C0 | 624 | X033A004p001U007p001A004p00 | 915 |
| 001570 | 04p000C007p000A004p000C007 | | 0G007p000A004p000U007p000G0 | |
| | p000C004p000U007p000U004p0 | | 04p000G007p000G004p000A007p | |
| | 00C007p000C004p000C007p000 | | 000A004p000G007p000G004p000 | |
| | A004p000U007p000C004p000U0 | | U007p000G004p000G007p000A00 | |
| | 07p001A004p001U007 | | 4p000U007p000C004p001C004p0 | |
| | | | 01G004 | |
| DT- | A007p001U004p001C007p000C0 | 625 | X033U004p001C007p001U004p00 | 916 |
| 001571 | 04p000A007p000C004p000C007 | | 0A007p000G004p000A007p000U0 | |
| | p000U004p000U007p000C004p0 | | 04p000G007p000G004p000G007p | |
| | 00C007p000C004p000A007p000 | | 000A004p000A007p000G004p000 | |
| | U004p000C007p000U004p000A0 | | G007p000U004p000G007p000G00 | |
| | 07p001G004p001A007 | | 4p000A007p000U004p001C004p0 | |
| | | | 01C004 | |
| DT- | U007p001C004p001C007p000A0 | 626 | X033A004p001U007p001C004p00 | 917 |
| 001572 | 04p000C007p000C004p000U007 | | 0U007p000A004p000G007p000A0 | |
| | p000U004p000C007p000C004p0 | | 04p000U007p000G004p000G007p | |
| | 00C007p000A004p000U007p000 | | 000G004p000A007p000A004p000 | |
| | C004p000U007p000A004p000G0 | | G007p000G004p000U007p000G00 | |
| | 07p001A004p001U007 | | 4p000G007p000A004p001U004p0 | |
| | | | 01C004 | |
| DT- | C007p001C004p001A007p000C0 | 627 | X033A004p001A007p001U004p00 | 918 |
| 001573 | 04p000C007p000U004p000U007 | | 0C007p000U004p000A007p000G0 | |
| | p000C004p000C007p000C004p0 | | 04p000A007p000U004p000G007p | |
| | 00A007p000U004p000C007p000 | | 000G004p000G007p000A004p000 | |
| | U004p000A007p000G004p000A0 | | A007p000G004p000G007p000U00 | |
| | 07p001U004p001U007 | | 4p000G007p000G004p001A004p0 | |
| | | | 01U004 | |
| DT- | A007p001C004p001C007p000U0 | 628 | X033A004p001U007p001G004p00 | 919 |
| 001574 | 04p000G007p000C004p000U007 | | 0G007p000C004p000A007p000G0 | |
| | p000G004p000C007p000U004p0 | | 04p000G007p000G004p000A007p | |
| | 00C007p000C004p000C007p000 | | 000G004p000C007p000A004p000 | |
| | U004p000G007p000C004p000C0 | | G007p000C004p000A007p000G00 | |
| | 07p001A004p001U007 | | 4p000G007p000U004p001G004p0 | |
| | | | 01G004 | |
| DT- | U007p001C004p001C007p000C0 | 629 | X033A004p001A007p001U004p00 | 920 |
| 001575 | 04p000U007p000A004p000G007 | | 0A007p000G004p000G007p000C0 | |
| | p000G004p000C007p000C004p0 | | 04p000C007p000A004p000G007p | |
| | 00U007p000G004p000G007p000 | | 000G004p000C007p000C004p000 | |
| | C004p000C007p000U004p000A0 | | U007p000A004p000G007p000G00 | |
| | 07p001U004p001U007 | | 4p000G007p000A004p001C004p0 | |
| | | | 01C004 | |
| DT- | A007p001U004p001U007p000C0 | 630 | X033A004p001C007p001A004pA0 | 921 |
| 001576 | 04p000C007p000U004p000C007 | | 07pU004pA007pA004pA007pU004 | |
| | p000G004p000G007p000U004p0 | | pA007pC004pC007pG004pA007pG | |
| | 00A007p000U004p000U007p000 | | 004pG007pA004pA007pU004p001 | |
| | U004p000A007p000U004p000U0 | | G004p001U004 | |
| | 07p001G004p001U007 | | | |
| DT- | U007p001U004p001C007p000C0 | 631 | X033A004p001A007p001C004p00 | 922 |
| 001577 | 04p000U007p000C004p000G007 | | 0A007p000A004p000U007p000A0 | |
| | p000G004p000U007p000A004p0 | | 04p000A007p000A004p000U007p | |
| | 00U007p000U004p000U007p000 | | 000A004p000C007p000C004p000 | |
| | A004p000U007p000U004p000G0 | | G007p000A004p000G007p000G00 | |
| | 07p001U004p001U007 | | 4p000A007p000A004p001U004p0 | |
| | | | 01G004 | |
| DT- | C007p001U004p001C007p000G0 | 632 | X033A004p001C007p001A004p00 | 923 |
| 001578 | 04p000G007p000U004p000A007 | | 0G007p000A004p000C007p000A0 | |
| | p000U004p000U007p000U004p0 | | 04p000A007p000U004p000A007p | |
| | 00A007p000U004p000U007p000 | | 000A004p000A007p000U004p000 | |
| | G004p000U007p000C004p000U0 | | A007p000C004p000C007p000G00 | |
| | 07p001G004p001U007 | | 4p000A007p000G004p001G004p0 | |
| | | | 01A004 | |
| DT- | U007p001C004p001G007p000G0 | 633 | X033A004p001A007p001C004p00 | 924 |
| 001579 | 04p000U007p000A004p000U007 | | 0A007p000G004p000A007p000C0 | |
| | p000U004p000U007p000A004p0 | | 04p000A007p000A004p000U007p | |
| | 00U007p000U004p000G007p000 | | 000A004p000A007p000A004p000 | |
| | U004p000C007p000U004p000G0 | | U007p000A004p000C007p000C00 | |
| | 07p001U004p001U007 | | 4p000G007p000A004p001G004p0 | |
| | | | 01G004 | |
| DT- | C007p001G004p001G007p000U0 | 634 | X033A004p001G007p001A004p00 | 925 |
| 001580 | 04p000A007p000U004p000U007 | | 0C007p000A004p000G007p000A0 | |
| | p000U004p000A007p000U004p0 | | 04p000C007p000A004p000A007p | |
| | 00U007p000G004p000U007p000 | | 0000004p000A007p000A004p000 | |
| | C004p000U007p000G004p000U0 | | A007p000U004p000A007p000C00 | |
| | 07p001C004p001U007 | | 4p000C007p000G004p001A004p0 | |
| | | | 01G004 | |
| DT- | G007p001C004p001G007p000G0 | 635 | X0330004p001C007p001A004p00 | 926 |
| 001581 | 04p000A007p000G004p000C007 | | 0C007p000C004p000A007p000C0 | |
| | p000C004p000C007p000A004p0 | | 04p000G007p000A004p000U007p | |
| | 00U007p000C004p000G007p000 | | 000G004p000G007p000G004p000 | |
| | U004p000G007p000G004p000U0 | | C007p000U004p000C007p000C00 | |
| | 07p001G004p001A007 | | 4p000G007p000C004p001U004p0 | |
| | | | 01G004 | |
| DT- | C007p001G004p001G007p000A0 | 636 | X033A004p001U007p001C004p00 | 927 |
| 001582 | 04p000G007p000C004p000C007 | | 0A007p000C004p000C007p000A0 | |
| | p000C004p000A007p000U004p0 | | 04p000C007p000G004p000A007p | |
| | 00C007p000G004p000U007p000 | | 0000004p000G007p000G004p000 | |
| | G004p000G007p000U004p000G0 | | G007p000C004p000U007p000C00 | |
| | 07p001A004p001U007 | | 4p000C007p000G004p001C004p0 | |
| | | | 010004 | |
| DT- | G007p001C004p001C007p000C0 | 637 | X033A004p001A007p001G004p00 | 928 |
| 001583 | 04p000A007p000U004p000C007 | | 0C007p000C004p000U007p000C0 | |
| | p000G004p000U007p000G004p0 | | 04p000A007p000C004p000C007p | |
| | 00G007p000U004p000G007p000 | | 000A004p000C007p000G004p000 | |
| | A004p000G007p000G004p000C0 | | A007p000U004p000G007p000G00 | |
| | 04p001U004p001U007 | | 4p000G007p000C004p001U004p0 | |
| | | | 01C004 | |
| DT- | C007p001C004p001C007p000A0 | 638 | X0330004p001A007p001A004p00 | 929 |
| 001584 | 04p000U007p000C004p000G007 | | 0G007p000C004p000C007p000U0 | |
| | p000U004p000G007p000G004p0 | | 04p000C007p000A004p000C007p | |
| | 00U007p000G004p000A007p000 | | 000C004p000A007p000C004p000 | |
| | G004p000G007p000C004p000U0 | | G007p000A004p000U007p000G00 | |
| | 07p001U004p001A007 | | 4p000G007p000G004p001C004p0 | |
| | | | 01U004 | |
| DT- | C007p001C004p001A007p000U0 | 639 | X0330004p001U007p001A004p00 | 930 |
| 001585 | 04p000C007p000G004p000U007 | | 0A007p000G004p000C007p000C0 | |
| | p000G004p000G007p000U004p0 | | 04p000U007p000C004p000A007p | |
| | 00G007p000A004p000G007p000 | | 000C004p000C007p000A004p000 | |
| | G004p000C007p000U004p000U0 | | C007p000G004p000A007p000U00 | |
| | 07p001A004p001A007 | | 4p000G007p000G004p001G004p0 | |
| | | | 01C004 | |
| DT- | C007p001A004p001U007p000C0 | 640 | X033A004p001U007p001U004p00 | 931 |
| 001586 | 04p000G007p000U004p000G007 | | 0A007p000A004p000G007p000C0 | |
| | p000G004p000U007p000G004p0 | | 04p000C007p000U004p000C007p | |
| | 00A007p000G004p000G007p000 | | 000A004p000C007p000C004p000 | |
| | C004p000U007p000U004p000A0 | | A007p000C004p000G007p000A00 | |
| | 07p001A004p001U007 | | 4p000U007p000G004p001G004p0 | |
| | | | 01G004 | |
| DT- | C007p001G004p001U007p000G0 | 641 | X033A004p001U007p001C004p00 | 932 |
| 001587 | 04p000G007p000U004p000G007 | | 0C007p000U004p000U007p000A0 | |
| | p000A004p000G007p000G004p0 | | 04p000A007p000G004p000C007p | |
| | 00C007p000U004p000U007p000 | | 000C004p000U007p000C004p000 | |
| | A004p000A007p000G004p000G0 | | A007p000C004p000C007p000A00 | |
| | 07p001A004p001U007 | | 4p000C007p000G004p001A004p0 | |
| | | | 010004 | |
| DT- | G007p001G004p001U007p000G0 | 642 | X033A004p001A007p001C004p00 | 933 |
| 001588 | 04p000A007p000G004p000G007 | | 0C007p000U004p000C007p000C0 | |
| | p000C004p000U007p000U004p0 | | 04p000U007p000U004p000A007p | |
| | 00A007p000A004p000G007p000 | | 000A004p000G007p000C004p000 | |
| | G004p000A007p000G004p000G0 | | C007p000U004p000C007p000A00 | |
| | 07p001U004p001U007 | | 4p000C007p000C004p001A004p0 | |
| | | | 01C004 | |
| DT- | A007p001G004p001G007p000G0 | 643 | X033A004p001A007p001A004p00 | 934 |
| 001589 | 04p000A007p000C004p000G007 | | 0U007p000C004p000U007p000C0 | |
| | p000A004p000C007p000U004p0 | | 04p000G007p000A004p000A007p | |
| | 00U007p000C004p000G007p000 | | 000G004p000U007p000C004p000 | |
| | A004p000G007p000A004p000U0 | | G007p000U004p000C007p000C00 | |
| | 07p001U004p001U007 | | 4p000C007p000U004p001C004p0 | |
| | | | 01U004 | |
| DT- | G007p001G004p001A007p000C0 | 644 | X033A004p001A007p001G004p00 | 935 |
| 001590 | 04p000G007p000A004p000C007 | | 0A007p000A004p000U007p000C0 | |
| | p000U004p000U007p000C004p0 | | 04p000U007p000C004p000G007p | |
| | 00G007p000A004p000G007p000 | | 000A004p000A007p000G004p000 | |
| | A004p000U007p000U004p000C0 | | U007p000C004p000G007p000U00 | |
| | 07p001U004p001U007 | | 4p000C007p000C004p001C004p0 | |
| | | | 01U004 | |
| DT- | C007p001G004p001A007p000C0 | 645 | X033A004p001U007p001U004p00 | 936 |
| 001591 | 04p000U007p000U004p000C007 | | 0C007p000A004p000G007p000A0 | |
| | p000G004p000A007p000G004p0 | | 04p000A007p000U004p000C007p | |
| | 00A007p000U004p000U007p000 | | 0000004p000C007p000G004p000 | |
| | C004p000U007p000G004p000A0 | | A007p000A004p000G007p000U00 | |
| | 07p001A004p001U007 | | 4p000C007p000G004p001U004p0 | |
| | | | 01C004 | |
| DT- | C007p001G004p001A007p000G0 | 646 | X033A004p001A007p001U004p00 | 937 |
| 001592 | 04p000A007p000U004p000U007 | | 0C007p000A004p000C007p000C0 | |
| | p000C004p000U007p000G004p0 | | 04p000U007p000U004p000C007p | |
| | 00A007p000A004p000G007p000 | | 000A004p000G007p000A004p000 | |
| | G004p000U007p000G004p000A0 | | A007p000U004p000C007p000U00 | |
| | 07p001U004p001U007 | | 4p000C007p000G004p001A004p0 | |
| | | | 01A004 | |
| DT- | A007p001U004p001U007p000C0 | 647 | X033A004p001U007p001C004p00 | 938 |
| 001593 | 04p000U007p000G004p000A007 | | 0C007p000G004p000A007p000U0 | |
| | p000A004p000G007p000G004p0 | | 04p000C007p000A004p000C007p | |
| | 00U007p000G004p000A007p000 | | 000C004p000U007p000U004p000 | |
| | U004p000C007p000G004p000G0 | | C007p000A004p000G007p000A00 | |
| | 07p001A004p001U007 | | 4p000A007p000U004p001C004p0 | |
| | | | 01U004 | |
| DT- | G007p001A004p001U007p000G0 | 648 | X033A004p001U007p001G004p00 | 939 |
| 001594 | 04p000A007p000A004p000G007 | | 0G007p000C004p000C007p000C0 | |
| | p000C004p000A007p000G004p0 | | 04p000G007p000U004p000C007p | |
| | 00A007p000C004p000G007p000 | | 0000004p000G007p000C004p000 | |
| | G004p000G007p000C004p000C0 | | U007p000U004p000C007p000A00 | |
| | 07p001A004p001U007 | | 4p000U007p000C004p001U004p0 | |
| | | | 010004 | |
| DT- | A007p001G004p001C007p000A0 | 649 | X0330004p001A007p001C004p00 | 940 |
| 001595 | 04p000G007p000A004p000C007 | | 0A007p000C004p000C007p000U0 | |
| | p000G004p000G007p000G004p0 | | 04p000G007p000G004p000C007p | |
| | 00C007p000C004p000A007p000 | | 000C004p000C007p000G004p000 | |
| | G004p000G007p000U004p000G0 | | U007p000C004p000U007p000G00 | |
| | 07p001U004p001A007 | | 4p000C007p000U004p001U004p0 | |
| | | | 01C004 | |
| DT- | C007p001A004p001U007p000G0 | 650 | X033A004p001U007p001U004p00 | 941 |
| 001596 | 04p000A007p000A004p000G007 | | 0G007p000U004p000U007p000C0 | |
| | p000A004p000U007p000C004p0 | | 04p000A007p000U004p000G007p | |
| | 00A007p000U004p000G007p000 | | 000A004p000U007p000C004p000 | |
| | A004p000A007p000C004p000A0 | | U007p000U004p000C007p000A00 | |
| | 07p001A004p001U007 | | 4p000U007p000G004p001G004p0 | |
| | | | 01C004 | |
| DT- | U007p001G004p001A007p000A0 | 651 | X033A004p001A007p001C004p00 | 942 |
| 001597 | 04p000G007p000A004p000U007 | | 0U007p000U004p000G007p000U0 | |
| | p000C004p000A007p000U004p0 | | 04p000U007p000C004p000A007p | |
| | 00G007p000A004p000A007p000 | | 0000004p000G007p000A004p000 | |
| | C004p000A007p000A004p000G0 | | U007p000C004p000U007p000U00 | |
| | 07p001U004p001U007 | | 4p000C007p000A004p001U004p0 | |
| | | | 01G004 | |
| DT- | C007p001A004p001U007p000G0 | 652 | X033A004p001A007p001U004p00 | 943 |
| 001598 | 04p000A007p000A004p000C007 | | 0G007p000U004p000C007p000C0 | |
| | p000A004p000A007p000G004p0 | | 04p000C007p000A004p000C007p | |
| | 00U007p000G004p000G007p000 | | 0000004p000U007p000G004p000 | |
| | G004p000A007p000C004p000A0 | | U007p000U004p000C007p000A00 | |
| | 07p001U004p001U007 | | 4p000U007p000G004p001A004p0 | |
| | | | 01U004 | |
| DT- | C007p001A004p001A007p000G0 | 653 | X033A004p001U007p001U004p00 | 944 |
| 001599 | 04p000U007p000G004p000G007 | | 0C007p000A004p000G007p000C0 | |
| | p000G004p000A007p000C004p0 | | 04p000A007p000U004p000G007p | |
| | 00A007p000U004p000G007p000 | | 000U004p000C007p000C004p000 | |
| | C004p000U007p000G004p000A0 | | C007p000A004p000C007p000U00 | |
| | 07p001A004p001U007 | | 4p000U007p000G004p001U004p0 | |
| | | | 01U004 | |
| DT- | G007p001A004p001G007p000G0 | 654 | X033A004p001A007p001C004p00 | 945 |
| 001600 | 04p000U007p000G004p000U007 | | 0G007p000G004p000A007p000A0 | |
| | p000C004p000G007p000U004p0 | | 04p000G007p000C004p000A007p | |
| | 00G007p000C004p000U007p000 | | 000C004p000G007p000A004p000 | |
| | U004p000C007p000C004p000G0 | | C007p000A004p000C007p000C00 | |
| | 07p001U004p001U007 | | 4p000U007p000C004p001G004p0 | |
| | | | 01C004 | |
| DT- | G007p001G004p001U007p000G0 | 655 | X033A004p001U007p001C004p00 | 946 |
| 001601 | 04p000U007p000C004p000G007 | | 0A007p000C004p000G007p000G0 | |
| | p000U004p000G007p000C004p0 | | 04p000A007p000A004p000G007p | |
| | 00U007p000U004p000C007p000 | | 000C004p000A007p000C004p000 | |
| | C004p000G007p000U004p000G0 | | G007p000A004p000C007p000A00 | |
| | 07p001A004p001U007 | | 4p000C007p000C004p001U004p0 | |
| | | | 01C004 | |
| DT- | C007p001G004p001U007p000G0 | 656 | X033A004p001U007p001C004p00 | 947 |
| 001602 | 04p000C007p000U004p000U007 | | 0U007p000C004p000C007p000U0 | |
| | p000C004p000C007p000G004p0 | | 04p000C007p000A004p000C007p | |
| | 00U007p000G004p000A007p000 | | 000G004p000G007p000A004p000 | |
| | G004p000G007p000A004p000G0 | | A007p000G004p000C007p000A00 | |
| | 07p001A004p001U007 | | 4p000C007p000G004p001A004p0 | |
| | | | 01C004 | |
| DT- | C007p001C004p001G007p000U0 | 657 | X033A004p001A007p001C004p00 | 948 |
| 001603 | 04p000G007p000A004p000G007 | | 0G007p000U004p000C007p000C0 | |
| | p000G004p000A007p000G004p0 | | 04p000C007p000U004p000C007p | |
| | 00A007p000G004p000G007p000 | | 0000004p000C007p000C004p000 | |
| | G004p000A007p000C004p000G0 | | U007p000C004p000A007p000C00 | |
| | 07p001U004p001U007 | | 4p000G007p000G004p001A004p0 | |
| | | | 01A004 | |
| DT- | G007p001U004p001G007p000A0 | 658 | X033A004p001A007p001C004p00 | 949 |
| 001604 | 04p000G007p000G004p000A007 | | 0A007p000C004p000G007p000U0 | |
| | p000G004p000A007p000G004p0 | | 04p000C007p000C004p000C007p | |
| | 00G007p000G004p000A007p000 | | 0000004p000C007p000U004p000 | |
| | C004p000G007p000U004p000G0 | | C007p000C004p000U007p000C00 | |
| | 07p001U004p001U007 | | 4p000A007p000C004p001G004p0 | |
| | | | 01G004 | |
| DT- | A007p001G004p001A007p000G0 | 659 | X0330004p001U007p001C004p00 | 950 |
| 001605 | 04p000G007p000G004p000A007 | | 0A007p000C004p000C007p000A0 | |
| | p000C004p000G007p000U004p0 | | 04p000A007p000C004p000A007p | |
| | 00G007p000U004p000U007p000 | | 000C004p000G007p000U004p000 | |
| | G004p000G007p000U004p000G0 | | C007p000C004p000C007p000U00 | |
| | 07p001A004p001A007 | | 4p000C007p000U004p001C004p0 | |
| | | | 01C004 | |
| DT- | G007p001A004p001G007p000G0 | 660 | X033A004p001U007p001U004p00 | 951 |
| 001606 | 04p000G007p000A004p000C007 | | 0C007p000A004p000C007p000C0 | |
| | p000G004p000U007p000G004p0 | | 04p000A007p000A004p000C007p | |
| | 00U007p000U004p000G007p000 | | 000A004p000C007p000G004p000 | |
| | G004p000U007p000G004p000A0 | | U007p000C004p000C007p000C00 | |
| | 07p001A004p001U007 | | 4p000U007p000C004p001U004p0 | |
| | | | 01C004 | |
| DT- | A007p001G004p001G007p000G0 | 661 | X033C004p001A007p001U004p00 | 952 |
| 001607 | 04p000A007p000C004p000G007 | | 0U007p000C004p000A007p000C0 | |
| | p000U004p000G007p000U004p0 | | 04p000C007p000A004p000A007p | |
| | 00U007p000G004p000G007p000 | | 000C004p000A007p000C004p000 | |
| | U004p000G007p000A004p000A0 | | G007p000U004p000C007p000C00 | |
| | 07p001U004p001G007 | | 4p000C007p000U004p001C004p0 | |
| | | | 01U004 | |
| DT- | G007p001G004p001U007p000G0 | 662 | X033U004p001G007p001C004p00 | 953 |
| 001608 | 04p000G007p000A004p000U007 | | 0A007p000G004p000C007p000U0 | |
| | p000C004p000A007p000C004p0 | | 04p000G007p000C004p000G007p | |
| | 00G007p000C004p000A007p000 | | 0000004p000G007p000A004p000 | |
| | G004p000C007p000U004p000G0 | | U007p000C004p000C007p000A00 | |
| | 07p001C004p001A007 | | 4p000C007p000C004p001G004p0 | |
| | | | 01C004 | |
| DT- | U007p001C004p001G007p000C0 | 663 | X0330004p001U007p001C004p00 | 954 |
| 001609 | 04p000C007p000U004p000U007 | | 0U007p000C004p000A007p000U0 | |
| | p000C004p000C007p000A004p0 | | 04p000C007p000C004p000U007p | |
| | 00G007p000G004p000A007p000 | | 000G004p000G007p000A004p000 | |
| | U004p000G007p000A004p000G0 | | A007p000G004p000G007p000C00 | |
| | 07p001A004p001A007 | | 4p000G007p000A004p001A004p0 | |
| | | | 01G004 | |
| DT- | C007p001G004p001C007p000C0 | 664 | X033A004p001U007p001U004p00 | 955 |
| 001610 | 04p000U007p000U004p000C007 | | 0C007p000U004p000C007p000A0 | |
| | p000C004p000A007p000G004p0 | | 04p000U007p000C004p000C007p | |
| | 00G007p000A004p000U007p000 | | 0000004p000G007p000G004p000 | |
| | G004p000A007p000G004p000A0 | | A007p000A004p000G007p000G00 | |
| | 07p001A004p001U007 | | 4p000C007p000G004p001A004p0 | |
| | | | 01A004 | |
| DT- | C007p001C004p001U007p000G0 | 665 | X033A004p001U007p001C004p00 | 956 |
| 001611 | 04p000U007p000A004p000C007 | | 0C007p000A004p000U007p000G0 | |
| | p000U004p000U007p000G004p0 | | 04p000A007p000C004p000C007p | |
| | 00G007p000U004p000C007p000 | | 000A004p000G007p000G004p000 | |
| | A004p000U007p000G004p000G0 | | U007p000A004p000C007p000A00 | |
| | 07p001A004p001U007 | | 4p000G007p000G004p001U004p0 | |
| | | | 01A004 | |
| DT- | C007p001U004p001G007p000U0 | 666 | X033A004p001C007p001U004p00 | 957 |
| 001612 | 04p000A007p000C004p000C007 | | 0C007p000C004p000A007p000U0 | |
| | p000U004p000G007p000G004p0 | | 04p000G007p000A004p000C007p | |
| | 00U007p000C004p000A007p000 | | 000C004p000A007p000G004p000 | |
| | 0004p000G007p000G004p000A0 | | G007p000U004p000A007p000C00 | |
| | 07p001G004p001U007 | | 4p000A007p000G004p001G004p0 | |
| | | | 01U004 | |
| DT- | U007p001G004p001U007p000A0 | 667 | X0330004p001A007p001C004p00 | 958 |
| 001613 | 04p000C007p000C004p000U007 | | 0U007p000C004p000C007p000A0 | |
| | p000G004p000G007p000U004p0 | | 04p000U007p000G004p000A007p | |
| | 00C007p000A004p000U007p000 | | 000C004p000C007p000A004p000 | |
| | G004p000G007p000A004p000G0 | | G007p000G004p000U007p000A00 | |
| | 07p001U004p001A007 | | 4p000C007p000A004p001G004p0 | |
| | | | 01G004 | |
| DT- | G007p001U004p001A007p000C0 | 668 | X033A004p001U007p001A004p00 | 959 |
| 001614 | 04p000C007p000U004p000G007 | | 0C007p000U004p000C007p000C0 | |
| | p000G004p000U007p000C004p0 | | 04p000A007p000U004p000G007p | |
| | 00A007p000U004p000G007p000 | | 000A004p000C007p000C004p000 | |
| | G004p000A007p000G004p000U0 | | A007p000G004p000G007p000U00 | |
| | 07p001A004p001U007 | | 4p000A007p000C004p001A004p0 | |
| | | | 01G004 | |
| DT- | C007p001C004p001U007p000G0 | 669 | X033A004p001U007p001A004p00 | 960 |
| 001615 | 04p000G007p000U004p000C007 | | 0A007p000U004p000A007p000C0 | |
| | p000A004p000U007p000G004p0 | | 04p000U007p000C004p000C007p | |
| | 00G007p000A004p000G007p000 | | 000A004p000U007p000G004p000 | |
| | U004p000A007p000U004p000U0 | | A007p000C004p000C007p000A00 | |
| | 07p001A004p001U007 | | 4p000G007p000G004p001U004p0 | |
| | | | 01A004 | |
| DT- | G007p001G004p001U007p000C0 | 670 | X033A004p001A007p001C004p00 | 961 |
| 001616 | 04p000A007p000U004p000G007 | | 0G007p000U004p000A007p000A0 | |
| | p000G004p000A007p000G004p0 | | 04p000U007p000A004p000C007p | |
| | 00U007p000A004p000U007p000 | | 0000004p000C007p000C004p000 | |
| | U004p000A007p000C004p000G0 | | A007p000U004p000G007p000A00 | |
| | 07p001U004p001U007 | | 4p000C007p000C004p001A004p0 | |
| | | | 01G004 | |
| DT- | G007p001G004p001A007p000C0 | 671 | X033A004p001A007p001G004p00 | 962 |
| 001617 | 04p000C007p000U004p000G007 | | 0C007p000A004p000G007p000U0 | |
| | p000C004p000U007p000G004p0 | | 04p000G007p000U004p000C007p | |
| | 00A007p000C004p000A007p000 | | 000A004p000G007p000C004p000 | |
| | C004p000U007p000G004p000C0 | | A007p000G004p000G007p000U00 | |
| | 07p001U004p001U007 | | 4p000C007p000C004p001C004p0 | |
| | | | 01C004 | |
| DT- | A007p001C004p001C007p000U0 | 672 | X033A004p001U007p001C004p00 | 963 |
| 001618 | 04p000G007p000C004p000U007 | | 0A007p000G004p000C007p000A0 | |
| | p000G004p000A007p000C004p0 | | 04p000G007p000U004p000G007p | |
| | 00A007p000C004p000U007p000 | | 0000004p000C007p000A004p000 | |
| | G004p000C007p000U004p000G0 | | G007p000C004p000A007p000G00 | |
| | 07p001A004p001U007 | | 4p000G007p000U004p001C004p0 | |
| | | | 01C004 | |
| DT- | U007p001G004p001C007p000U0 | 673 | X0330004p001U007p001G004p00 | 964 |
| 001619 | 04p000G007p000A004p000C007 | | 0C007p000U004p000C007p000A0 | |
| | p000A004p000C007p000U004p0 | | 04p000G007p000C004p000A007p | |
| | 00G007p000C004p000U007p000 | | 000G004p000U007p000G004p000 | |
| | G004p000A007p000G004p000C0 | | U007p000C004p000A007p000G00 | |
| | 07p001A004p001A007 | | 4p000C007p000A004p001G004p0 | |
| | | | 01G004 | |
| DT- | G007p001C004p001U007p000G0 | 674 | X033A004p001U007p001U004p00 | 965 |
| 001620 | 04p000A007p000C004p000A007 | | 0G007p000C004p000U007p000C0 | |
| | p000C004p000U007p000G004p0 | | 04p000A007p000G004p000C007p | |
| | 00C007p000U004p000G007p000 | | 000A004p000G007p000U004p000 | |
| | A004p000G007p000C004p000A0 | | G007p000U004p000C007p000A00 | |
| | 07p001A004p001U007 | | 4p000G007p000C004p001A004p0 | |
| | | | 01G004 | |
| DT- | C007p001U004p001G007p000A0 | 675 | X033A004p001C007p001U004p00 | 966 |
| 001621 | 04p000C007p000A004p000C007 | | 0U007p000G004p000C007p000U0 | |
| | p000U004p000G007p000C004p0 | | 04p000C007p000A004p000G007p | |
| | 00U007p000G004p000A007p000 | | 000C004p000A007p000G004p000 | |
| | G004p000C007p000A004p000A0 | | U007p000G004p000U007p000C00 | |
| | 07p001G004p001U007 | | 4p000A007p000G004p001C004p0 | |
| | | | 01A004 | |
| DT- | U007p001G004p001A007p000C0 | 676 | X033A004p001A007p001C004p00 | 967 |
| 001622 | 04p000A007p000C004p000U007 | | 0U007p000U004p000G007p000C0 | |
| | p000G004p000C007p000U004p0 | | 04p000U007p000C004p000A007p | |
| | 00G007p000A004p000G007p000 | | 000G004p000C007p000A004p000 | |
| | C004p000A007p000A004p000G0 | | G007p000U004p000G007p000U00 | |
| | 07p001U004p001U007 | | 4p000C007p000A004p001G004p0 | |
| | | | 01C004 | |
| DT- | A007p001C004p001A007p000C0 | 677 | X0330004p001A007p001A004p00 | 968 |
| 001623 | 04p000U007p000G004p000C007 | | 0A007p000C004p000U007p000U0 | |
| | p000U004p000G007p000A004p0 | | 04p000G007p000C004p000U007p | |
| | 00G007p000C004p000A007p000 | | 000C004p000A007p000G004p000 | |
| | A004p000G007p000U004p000U0 | | C007p000A004p000G007p000U00 | |
| | 07p001U004p001A007 | | 4p000G007p000U004p001C004p0 | |
| | | | 01A004 | |
| DT- | G007p001A004p001G007p000C0 | 678 | X033U004p001C007p001U004p00 | 969 |
| 001624 | 04p000G007p000G004p000A007 | | 0C007p000G004p000G007p000C0 | |
| | p000U004p000U007p000C004p0 | | 04p000C007p000G004p000G007p | |
| | 00C007p000G004p000G007p000 | | 000A004p000A007p000U004p000 | |
| | C004p000C007p000G004p000A0 | | C007p000C004p000G007p000C00 | |
| | 07p001G004p001A007 | | 4p000U007p000C004p001C004p0 | |
| | | | 01C004 | |
| DT- | A007p001G004p001C007p000G0 | 679 | X033A004p001U007p001C004p00 | 970 |
| 001625 | 04p000G007p000A004p000U007 | | 0U007p000C004p000G007p000G0 | |
| | p000U004p000C007p000C004p0 | | 04p000C007p000C004p000G007p | |
| | 00G007p000G004p000C007p000 | | 000G004p000A007p000A004p000 | |
| | C004p000G007p000A004p000G0 | | U007p000C004p000C007p000G00 | |
| | 07p001A004p001U007 | | 4p000C007p000U004p001C004p0 | |
| | | | 01C004 | |
| DT- | C007p001G004p001G007p000C0 | 680 | X033A004p001A007p001G004p00 | 971 |
| 001626 | 04p000C007p000G004p000A007 | | 0C007p000G004p000C007p000G0 | |
| | p000G004p000A007p000U004p0 | | 04p000C007p000C004p000A007p | |
| | 00G007p000G004p000C007p000 | | 0000004p000C007p000U004p000 | |
| | G004p000C007p000G004p000C0 | | C007p000G004p000G007p000C00 | |
| | 07p001U004p001U007 | | 4p000C007p000G004p001G004p0 | |
| | | | 01A004 | |
| DT- | G007p001G004p001C007p000C0 | 681 | X033U004p001A007p001A004p00 | 972 |
| 001627 | 04p000G007p000A004p000G007 | | 0G007p000C004p000G007p000C0 | |
| | p000A004p000U007p000G004p0 | | 04p000G007p000C004p000C007p | |
| | 00G007p000C004p000G007p000 | | 000A004p000U007p000C004p000 | |
| | C004p000G007p000C004p000U0 | | U007p000C004p000G007p000G00 | |
| | 07p001U004p001A007 | | 4p000C007p000C004p001G004p0 | |
| | | | 01G004 | |
| DT- | G007p001C004p001C007p000G0 | 682 | X033A004p001G007p001A004p00 | 973 |
| 001628 | 04p000A007p000G004p000A007 | | 0A007p000G004p000C007p000G0 | |
| | p000U004p000G007p000G004p0 | | 04p000C007p000G004p000C007p | |
| | 00C007p000G004p000C007p000 | | 000C004p000A007p000U004p000 | |
| | G004p000C007p000U004p000U0 | | C007p000U004p000C007p000G00 | |
| | 07p001C004p001U007 | | 4p000G007p000C004p001C004p0 | |
| | | | 01G004 | |
| DT- | C007p001C004p001G007p000A0 | 683 | X033U004p001A007p001G004p00 | 974 |
| 001629 | 04p000G007p000A004p000U007 | | 0A007p000A004p000G007p000C0 | |
| | p000G004p000G007p000C004p0 | | 04p000G007p000C004p000G007p | |
| | 00G007p000C004p000G007p000 | | 000C004p000C007p000A004p000 | |
| | C004p000U007p000U004p000C0 | | U007p000C004p000U007p000C00 | |
| | 07p001U004p001A007 | | 4p000G007p000G004p001C004p0 | |
| | | | 01C004 | |
| DT- | C007p001G004p001A007p000G0 | 684 | X033A004p001U007p001A004p00 | 975 |
| 001630 | 04p000A007p000U004p000G007 | | 0G007p000A004p000A007p000G0 | |
| | p000G004p000C007p000G004p0 | | 04p000C007p000G004p000C007p | |
| | 00C007p000G004p000C007p000 | | 000G004p000C007p000C004p000 | |
| | U004p000U007p000C004p000U0 | | A007p000U004p000C007p000U00 | |
| | 07p001A004p001U007 | | 4p000C007p000G004p001G004p0 | |
| | | | 01C004 | |
| DT- | G007p001C004p001U007p000U0 | 685 | X033A004p001U007p001C004p00 | 976 |
| 001631 | 04p000C007p000U004p000A007 | | 0U007p000C004p000C007p000G0 | |
| | p000C004p000C007p000U004p0 | | 04p000C007p000C004p000A007p | |
| | 00G007p000G004p000C007p000 | | 000G004p000G007p000U004p000 | |
| | G004p000G007p000A004p000G0 | | A007p000G004p000A007p000A00 | |
| | 07p001A004p001U007 | | 4p000G007p000C004p001G004p0 | |
| | | | 01C004 | |
| DT- | C007p001U004p001U007p000C0 | 686 | X033A004p001A007p001U004p00 | 977 |
| 001632 | 04p000U007p000A004p000C007 | | 0C007p000U004p000C007p000C0 | |
| | p000C004p000U007p000G004p0 | | 04p000G007p000C004p000C007p | |
| | 00G007p000C004p000G007p000 | | 000A004p000G007p000G004p000 | |
| | G004p000A007p000G004p000A0 | | U007p000A004p000G007p000A00 | |
| | 07p001U004p001U007 | | 4p000A007p000G004p001C004p0 | |
| | | | 01G004 | |
| DT- | U007p001U004p001C007p000U0 | 687 | X0330004p001A007p001A004p00 | 978 |
| 001633 | 04p000A007p000C004p000C007 | | 0U007p000C004p000U007p000C0 | |
| | p000U004p000G007p000G004p0 | | 04p000C007p000G004p000C007p | |
| | 00C007p000G004p000G007p000 | | 000C004p000A007p000G004p000 | |
| | A004p000G007p000A004p000U0 | | G007p000U004p000A007p000G00 | |
| | 07p001U004p001A007 | | 4p000A007p000A004p001G004p0 | |
| | | | 01C004 | |
| DT- | U007p001C004p001U007p000A0 | 688 | X033A004p001C007p001A004p00 | 979 |
| 001634 | 04p000C007p000C004p000U007 | | 0A007p000U004p000C007p000U0 | |
| | p000G004p000G007p000C004p0 | | 04p000C007p000C004p000G007p | |
| | 00G007p000G004p000A007p000 | | 000C004p000C007p000A004p000 | |
| | G004p000A007p000U004p000U0 | | G007p000G004p000U007p000A00 | |
| | 07p001G004p001U007 | | 4p000G007p000A004p001A004p0 | |
| | | | 01G004 | |
| DT- | C007p001U004p001A007p000C0 | 689 | X033A004p001A007p001C004p00 | 980 |
| 001635 | 04p000C007p000U004p000G007 | | 0A007p000A004p000U007p000C0 | |
| | p000G004p000C007p000G004p0 | | 04p000U007p000C004p000C007p | |
| | 00G007p000A004p000G007p000 | | 000G004p000C007p000C004p000 | |
| | A004p000U007p000U004p000G0 | | A007p000G004p000G007p000U00 | |
| | 07p001U004p001U007 | | 4p000A007p000G004p001A004p0 | |
| | | | 01A004 | |
| DT- | A007p001C004p001C007p000U0 | 690 | X033A004p001U007p001G004p00 | 981 |
| 001636 | 04p000G007p000G004p000C007 | | 0A007p000C004p000A007p000A0 | |
| | p000G004p000G007p000A004p0 | | 04p000U007p000C004p000U007p | |
| | 00G007p000A004p000U007p000 | | 000C004p000C007p000G004p000 | |
| | 0004p000G007p000U004p000C0 | | C007p000C004p000A007p000G00 | |
| | 07p001A004p001U007 | | 4p000G007p000U004p001A004p0 | |
| | | | 01G004 | |
| DT- | C007p001C004p001U007p000G0 | 691 | X033C004p001A007p001U004p00 | 982 |
| 001637 | 04p000G007p000C004p000G007 | | 0G007p000A004p000C007p000A0 | |
| | p000G004p000A007p000G004p0 | | 04p000A007p000U004p000C007p | |
| | 00A007p000U004p000U007p000 | | 000U004p000C007p000C004p000 | |
| | G004p000U007p000C004p000A0 | | G007p000C004p000C007p000A00 | |
| | 07p001U004p001G007 | | 4p000G007p000G004p001U004p0 | |
| | | | 01A004 | |
| DT- | C007p001G004p001G007p000A0 | 692 | X033A004p001U007p001G004p00 | 983 |
| 001638 | 04p000G007p000A004p000U007 | | 0G007p000C004p000C007p000A0 | |
| | p000U004p000G007p000U004p0 | | 04p000U007p000G004p000A007p | |
| | 00C007p000A004p000U007p000 | | 000C004p000A007p000A004p000 | |
| | G004p000G007p000C004p000C0 | | U007p000C004p000U007p000C00 | |
| | 07p001A004p001U007 | | 4p000C007p000G004p001C004p0 | |
| | | | 01C004 | |
| DT- | G007p001G004p001A007p000G0 | 693 | X0330004p001A007p001U004p00 | 984 |
| 001639 | 04p000A007p000U004p000U007 | | 0G007p000G004p000C007p000C0 | |
| | p000G004p000U007p000C004p0 | | 04p000A007p000U004p000G007p | |
| | 00A007p000U004p000G007p000 | | 000A004p000C007p000A004p000 | |
| | G004p000C007p000C004p000A0 | | A007p000U004p000C007p000U00 | |
| | 07p001U004p001A007 | | 4p000C007p000C004p001G004p0 | |
| | | | 01C004 | |
| DT- | G007p001G004p001G007p000A0 | 694 | X033U004p001U007p001G004p00 | 985 |
| 001640 | 04p000C007p000A004p000U007 | | 0U007p000C004p000G007p000G0 | |
| | p000C004p000A007p000A004p0 | | 04p000G007p000U004p000U007p | |
| | 00A007p000C004p000C007p000 | | 000U004p000G007p000A004p000 | |
| | C004p000G007p000A004p000C0 | | U007p000G004p000U007p000C00 | |
| | 07p001A004p001A007 | | 4p000C007p000C004p001U004p0 | |
| | | | 01G004 | |
| DT- | G007p001G004p001A007p000C0 | 695 | X033A004p001U007p001U004p00 | 986 |
| 001641 | 04p000A007p000U004p000C007 | | 0G007p000U004p000C007p000G0 | |
| | p000A004p000A007p000A004p0 | | 04p000G007p000G004p000U007p | |
| | 00C007p000C004p000C007p000 | | 000U004p000U007p000G004p000 | |
| | G004p000A007p000C004p000A0 | | A007p000U004p000G007p000U00 | |
| | 07p001A004p001U007 | | 4p000C007p000C004p001C004p0 | |
| | | | 01U004 | |
| DT- | G007p001A004p001C007p000A0 | 696 | X0330004p001G007p001U004p00 | 987 |
| 001642 | 04p000U007p000C004p000A007 | | 0U007p000G004p000U007p000C0 | |
| | p000A004p000A007p000C004p0 | | 04p000G007p000G004p000G007p | |
| | 00C007p000C004p000G007p000 | | 000U004p000U007p000U004p000 | |
| | A004p000C007p000A004p000A0 | | G007p000A004p000U007p000G00 | |
| | 07p001C004p001A007 | | 4p000U007p000C004p001C004p0 | |
| | | | 01C004 | |
| DT- | A007p001C004p001A007p000U0 | 697 | X033A004p001U007p001G004p00 | 988 |
| 001643 | 04p000C007p000A004p000A007 | | 0U007p000U004p000G007p000U0 | |
| | p000A004p000C007p000C004p0 | | 04p000C007p000G004p000G007p | |
| | 00C007p000G004p000A007p000 | | 000G004p000U007p000U004p000 | |
| | C004p000A007p000A004p000C0 | | U007p000G004p000A007p000U00 | |
| | 07p001A004p001U007 | | 4p000G007p000U004p001C004p0 | |
| | | | 01C004 | |
| DT- | C007p001A004p001U007p000C0 | 698 | X033A004p001A007p001U004p00 | 989 |
| 001644 | 04p000A007p000A004p000A007 | | 0G007p000U004p000U007p000G0 | |
| | p000C004p000C007p000C004p0 | | 04p000U007p000C004p000G007p | |
| | 00G007p000A004p000C007p000 | | 000G004p000G007p000U004p000 | |
| | A004p000A007p000C004p000A0 | | U007p000U004p000G007p000A00 | |
| | 07p001U004p001U007 | | 4p000U007p000G004p001U004p0 | |
| | | | 01C004 | |
| DT- | A007p001C004p001C007p000C0 | 699 | X033A004p001A007p001G004p00 | 990 |
| 001645 | 04p000G007p000A004p000C007 | | 0C007p000A004p000G007p000G0 | |
| | p000A004p000A007p000C004p0 | | 04p000A007p000U004p000G007p | |
| | 00A007p000U004p000C007p000 | | 000U004p000U007p000G004p000 | |
| | C004p000U007p000G004p000C0 | | U007p000C004p000G007p000G00 | |
| | 07p001U004p001U007 | | 4p000G007p000U004p001U004p0 | |
| | | | 010004 | |
| DT- | C007p001G004p001A007p000C0 | 700 | X033A004p001U007p001C004p00 | 991 |
| 001646 | 04p000A007p000A004p000C007 | | 0C007p000A004p000G007p000C0 | |
| | p000A004p000U007p000C004p0 | | 04p000A007p000G004p000G007p | |
| | 00C007p000U004p000G007p000 | | 000A004p000U007p000G004p000 | |
| | C004p000U007p000G004p000G0 | | U007p000U004p000G007p000U00 | |
| | 07p001A004p001U007 | | 4p000C007p000G004p001G004p0 | |
| | | | 01G004 | |
| DT- | A007p001C004p001A007p000U0 | 701 | X033A004p001A007p001G004p00 | 992 |
| 001647 | 04p000C007p000C004p000U007 | | 0C007p000G004p000G007p000U0 | |
| | p000G004p000C007p000U004p0 | | 04p000C007p000C004p000A007p | |
| | 00G007p000G004p000A007p000 | | 000G004p000C007p000A004p000 | |
| | C004p000C007p000G004p000C0 | | G007p000G004p000A007p000U00 | |
| | 07p001U004p001U007 | | 4p000G007p000U004p001U004p0 | |
| | | | 01G004 | |
| DT- | A007p001U004p001C007p000C0 | 702 | X033A004p001A007p001C004p00 | 993 |
| 001648 | 04p000U007p000G004p000C007 | | 0A007p000G004p000C007p000G0 | |
| | p000U004p000G007p000G004p0 | | 04p000G007p000U004p000C007p | |
| | 00A007p000C004p000C007p000 | | 000C004p000A007p000G004p000 | |
| | G004p000C007p000U004p000G0 | | C007p000A004p000G007p000G00 | |
| | 07p001U004p001U007 | | 4p000A007p000U004p001G004p0 | |
| | | | 01U004 | |
| DT- | U007p001G004p001G007p000A0 | 703 | X033A004p001U007p001G004p00 | 994 |
| 001649 | 04p000C007p000C004p000G007 | | 0U007p000G004p000G007p000C0 | |
| | p000C004p000U007p000G004p0 | | 04p000C007p000A004p000C007p | |
| | 00U007p000G004p000G007p000 | | 000A004p000G007p000C004p000 | |
| | C004p000C007p000A004p000C0 | | G007p000G004p000U007p000C00 | |
| | 07p001A004p001U007 | | 4p000C007p000A004p001G004p0 | |
| | | | 01C004 | |
| DT- | G007p001G004p001A007p000C0 | 704 | X033A004p001A007p001U004p00 | 995 |
| 001650 | 04p000C007p000G004p000C007 | | 0G007p000U004p000G007p000G0 | |
| | p000U004p000G007p000U004p0 | | 04p000C007p000C004p000A007p | |
| | 00G007p000G004p000C007p000 | | 000C004p000A007p000G004p000 | |
| | C004p000A007p000C004p000A0 | | C007p000G004p000G007p000U00 | |
| | 07p001U004p001U007 | | 4p000C007p000C004p001A004p0 | |
| | | | 01G004 | |
| DT- | C007p001A004p001C007p000A0 | 705 | X033A004p001G007p001U004p00 | 996 |
| 001651 | 04p000U007p000C004p000C007 | | 0C007p000G004p000G007p000C0 | |
| | p000G004p000C007p000C004p0 | | 04p000C007p000A004p000G007p | |
| | 00U007p000G004p000G007p000 | | 000G004p000C007p000G004p000 | |
| | C004p000C007p000G004p000A0 | | G007p000A004p000U007p000G00 | |
| | 07p001C004p001U007 | | 4p000U007p000G004p001G004p0 | |
| | | | 01C004 | |
| DT- | A007p001C004p001A007p000U0 | 706 | X033A004p001A007p001G004p00 | 997 |
| 001652 | 04p000C007p000C004p000G007 | | 0U007p000C004p000G007p000G0 | |
| | p000C004p000C007p000U004p0 | | 04p000C007p000C004p000A007p | |
| | 00G007p000G004p000C007p000 | | 000G004p000G007p000C004p000 | |
| | C004p000G007p000A004p000C0 | | G007p000G004p000A007p000U00 | |
| | 07p001U004p001U007 | | 4p000G007p000U004p001G004p0 | |
| | | | 01G004 | |
| DT- | C007p001A004p001U007p000C0 | 707 | X033G004p001A007p001A004p00 | 998 |
| 001653 | 04p000C007p000G004p000C007 | | 0G007p000U004p000C007p000G0 | |
| | p000C004p000U007p000G004p0 | | 04p000G007p000C004p000C007p | |
| | 00G007p000C004p000C007p000 | | 000A004p000G007p000G004p000 | |
| | G004p000A007p000C004p000U0 | | C007p000G004p000G007p000A00 | |
| | 07p001U004p001C007 | | 4p000U007p000G004p001U004p0 | |
| | | | 01G004 | |
| DT- | C007p001A004p001G007p000A0 | 708 | X033A004p001A007p001C004p00 | 999 |
| 001654 | 04p000U007p000G004p000G007 | | 0C007p000G004p000C007p000A0 | |
| | p000A004p000A007p000C004p0 | | 04p000C007p000C004p000G007p | |
| | 00G007p000G004p000U007p000 | | 000U004p000U007p000C004p000 | |
| | G004p000C007p000G004p000G0 | | C007p000A004p000U007p000C00 | |
| | 07p001U004p001U007 | | 4p000U007p000G004p001C004p0 | |
| | | | 01C004 | |
| DT- | C007p001G004p001A007p000G0 | 709 | X033A004p001A007p001C004p00 | 1000 |
| 001655 | 04p000A007p000U004p000C007 | | 0A007p000G004p000C007p000C0 | |
| | p000C004p000U007p000G004p0 | | 04p000U007p000G004p000C007p | |
| | 00C007p000A004p000G007p000 | | 000A004p000G007p000G004p000 | |
| | G004p000C007p000U004p000G0 | | A007p000U004p000C007p000U00 | |
| | 07p001U004p001U007 | | 4p000C007p000G004p001G004p0 | |
| | | | 01G004 | |
| DT- | G007p001G004p001C007p000C0 | 710 | X0330004p001A007p001G004p00 | 1001 |
| 001656 | 04p000U007p000G004p000G007 | | 0C007p000U004p000G007p000C0 | |
| | p000G004p000A007p000C004p0 | | 04p000C007p000U004p000G007p | |
| | 00A007p000G004p000G007p000 | | 000U004p000C007p000C004p000 | |
| | C004p000A007p000G004p000C0 | | C007p000A004p000G007p000G00 | |
| | 07p001U004p001A007 | | 4p000C007p000C004p001C004p0 | |
| | | | 01A004 | |
| DT- | G007p001C004p001C007p000U0 | 711 | X033A004p001U007p001A004p00 | 1002 |
| 001657 | 04p000G007p000G004p000G007 | | 0G007p000C004p000U007p000G0 | |
| | p000A004p000C007p000A004p0 | | 04p000C007p000C004p000U007p | |
| | 00G007p000G004p000C007p000 | | 000G004p000U007p000C004p000 | |
| | A004p000G007p000C004p000U0 | | C007p000C004p000A007p000G00 | |
| | 07p001A004p001U007 | | 4p000G007p000C004p001C004p0 | |
| | | | 01C004 | |
| DT- | U007p001U004p001C007p000G0 | 712 | X033A004p001G007p001A004p00 | 1003 |
| 001658 | 04p000C007p000C004p000U007 | | 0A007p000C004p000A007p000U0 | |
| | p000A004p000U007p000G004p0 | | 04p000U007p000U004p000C007p | |
| | 00A007p000A004p000A007p000 | | 000A004p000U007p000A004p000 | |
| | U004p000G007p000U004p000U0 | | G007p000G004p000C007p000G00 | |
| | 07p001C004p001U007 | | 4p000A007p000A004p001U004p0 | |
| | | | 01A004 | |
| DT- | U007p001C004p001G007p000C0 | 713 | X0330004p001A007p001G004p00 | 1004 |
| 001659 | 04p000C007p000U004p000A007 | | 0A007p000A004p000C007p000A0 | |
| | p000U004p000G007p000A004p0 | | 04p000U007p000U004p000U007p | |
| | 00A007p000A004p000U007p000 | | 000C004p000A007p000U004p000 | |
| | G004p000U007p000U004p000C0 | | A007p000G004p000G007p000C00 | |
| | 07p001U004p001A007 | | 4p000G007p000A004p001A004p0 | |
| | | | 01U004 | |
| DT- | G007p001G004p001G007p000C0 | 714 | X033A004p001G007p001U004p00 | 1005 |
| 001660 | 04p000A007p000G004p000A007 | | 0A007p000G004p000A007p000A0 | |
| | p000C004p000G007p000C004p0 | | 04p000G007p000G004p000G007p | |
| | 00C007p000C004p000U007p000 | | 000C004p000G007p000U004p000 | |
| | U004p000C007p000U004p000A0 | | C007p000U004p000G007p000C00 | |
| | 07p001C004p001U007 | | 4p000C007p000C004p001A004p0 | |
| | | | 01U004 | |
| DT- | G007p001A004p001C007p000G0 | 715 | X033A004p001U007p001C004p00 | 1006 |
| 001661 | 04p000C007p000C004p000C007 | | 0C007p000G004p000C007p000G0 | |
| | p000U004p000U007p000C004p0 | | 04p000U007p000A004p000G007p | |
| | 00U007p000A004p000C007p000 | | 000A004p000A007p000G004p000 | |
| | G004p000C007p000G004p000G0 | | G007p000G004p000C007p000G00 | |
| | 07p001A004p001U007 | | 4p000U007p000C004p001U004p0 | |
| | | | 01G004 | |
| DT- | A007p001C004p001G007p000C0 | 716 | X033A004p001U007p001C004p00 | 1007 |
| 001662 | 04p000C007p000C004p000U007 | | 0C007p000G004p000C007p000G0 | |
| | p000U004p000C007p000U004p0 | | 04p000U007p000A004p000G007p | |
| | 00A007p000C004p000G007p000 | | 000A004p000A007p000G004p000 | |
| | C004p000G007p000G004p000A0 | | G007p000G004p000C007p000G00 | |
| | 07p001U004p001U007 | | 4p000U007p000C004p001U004p0 | |
| | | | 01G004 | |
| DT- | C007p001G004p001C007p000C0 | 717 | X033A004p001A007p001A004pU0 | 1008 |
| 001663 | 04p000C007p000U004p000U007 | | 07pC004pC007pG004pC007pG004 | |
| | p000C004p000U007p000A004p0 | | pU007pA004pG007pA004pA007pG | |
| | 00C007p000G004p000C007p000 | | 004pG007pG004pC007pG004p001 | |
| | G004p000G007p000A004p000U0 | | U004p001C004 | |
| | 07p001U004p001U007 | | | |
| DT- | G007p001C004p001C007p000C0 | 718 | X033A004p001G007p001A004p00 | 1009 |
| 001664 | 04p000U007p000U004p000C007 | | 0A007p000U004p000C007p000C0 | |
| | p000U004p000A007p000C004p0 | | 04p000G007p000C004p000G007p | |
| | 00G007p000C004p000G007p000 | | 000U004p000A007p000G004p000 | |
| | G004p000A007p000U004p000U0 | | A007p000A004p000G007p000G00 | |
| | 07p001C004p001U007 | | 4p000G007p000C004p001G004p0 | |
| | | | 01U004 | |
| DT- | C007p001C004p001U007p000U0 | 719 | X033A004p001U007p001G004p00 | 1010 |
| 001665 | 04p000C007p000U004p000A007 | | 0G007p000A004p000A007p000U0 | |
| | p000C004p000G007p000C004p0 | | 04p000C007p000C004p000G007p | |
| | 00G007p000G004p000A007p000 | | 000C004p000G007p000U004p000 | |
| | 0004p000U007p000C004p000C0 | | A007p000G004p000A007p000A00 | |
| | 07p001A004p001U007 | | 4p000G007p000G004p001G004p0 | |
| | | | 01C004 | |
| DT- | C007p001G004p001G007p000A0 | 720 | X033A004p001U007p001C004p00 | 1011 |
| 001666 | 04p000U007p000U004p000C007 | | 0U007p000C004p000C007p000G0 | |
| | p000C004p000A007p000C004p0 | | 04p000C007p000C004p000G007p | |
| | 00G007p000G004p000C007p000 | | 000U004p000G007p000G004p000 | |
| | G004p000G007p000A004p000G0 | | A007p000A004p000U007p000C00 | |
| | 07p001A004p001U007 | | 4p000C007p000G004p001C004p0 | |
| | | | 01G004 | |
| DT- | A007p001U004p001U007p000C0 | 721 | X033U004p001A007p001G004p00 | 1012 |
| 001667 | 04p000C007p000A004p000C007 | | 0G007p000U004p000C007p000U0 | |
| | p000G004p000G007p000C004p0 | | 04p000C007p000C004p000G007p | |
| | 00G007p000G004p000A007p000 | | 000C004p000C007p000G004p000 | |
| | G004p000A007p000C004p000C0 | | U007p000G004p000G007p000A00 | |
| | 07p001U004p001A007 | | 4p000A007p000U004p001C004p0 | |
| | | | 01C004 | |
| DT- | G007p001G004p001C007p000A0 | 722 | X0330004p001G007p001U004p00 | 1013 |
| 001668 | 04p000A007p000G004p000A007 | | 0A007p000G004p000U007p000G0 | |
| | p000U004p000C007p000G004p0 | | 04p000G007p000A004p000C007p | |
| | 00U007p000C004p000C007p000 | | 000G004p000A007p000U004p000 | |
| | A004p000C007p000U004p000A0 | | C007p000U004p000U007p000G00 | |
| | 07p001C004p001A007 | | 4p000C007p000C004p001A004p0 | |
| | | | 01U004 | |
| DT- | G007p001C004p001A007p000A0 | 723 | X0330004p001U007p001G004p00 | 1014 |
| 001669 | 04p000G007p000A004p000U007 | | 0U007p000A004p000G007p000U0 | |
| | p000C004p000G007p000U004p0 | | 04p000G007p000G004p000A007p | |
| | 00C007p000C004p000A007p000 | | 000C004p000G007p000A004p000 | |
| | C004p000U007p000A004p000C0 | | U007p000C004p000U007p000U00 | |
| | 07p001A004p001A007 | | 4p000G007p000C004p001C004p0 | |
| | | | 01A004 | |
| DT- | C007p001A004p001A007p000G0 | 724 | X033C004p001U007p001U004p00 | 1015 |
| 001670 | 04p000A007p000U004p000C007 | | 0G007p000U004p000A007p000G0 | |
| | p000G004p000U007p000C004p0 | | 04p000U007p000G004p000G007p | |
| | 00C007p000A004p000C007p000 | | 000A004p000C007p000G004p000 | |
| | U004p000A007p000C004p000A0 | | A007p000U004p000C007p000U00 | |
| | 07p001A004p001G007 | | 4p000U007p000G004p001C004p0 | |
| | | | 01C004 | |
| DT- | G007p001A004p001U007p000C0 | 725 | X033A004p001U007p001C004p00 | 1016 |
| 001671 | 04p000G007p000U004p000C007 | | 0C007p000U004p000U007p000G0 | |
| | p000C004p000A007p000C004p0 | | 04p000U007p000A004p000G007p | |
| | 00U007p000A004p000C007p000 | | 000U004p000G007p000G004p000 | |
| | A004p000A007p000G004p000G0 | | A007p000C004p000G007p000A00 | |
| | 07p001A004p001U007 | | 4p000U007p000C004p001U004p0 | |
| | | | 010004 | |
| DT- | C007p001G004p001U007p000C0 | 726 | X033A004p001U007p001G004p00 | 1017 |
| 001672 | 04p000C007p000A004p000C007 | | 0C007p000U004p000C007p000C0 | |
| | p000U004p000A007p000C004p0 | | 04p000U007p000U004p000G007p | |
| | 00A007p000A004p000G007p000 | | 000U004p000A007p000G004p000 | |
| | G004p000A007p000G004p000C0 | | U007p000G004p000G007p000A00 | |
| | 07p001A004p001U007 | | 4p000C007p000G004p001A004p0 | |
| | | | 01U004 | |
| DT- | C007p001C004p001A007p000C0 | 727 | X033A004p001A007p001G004p00 | 1018 |
| 001673 | 04p000U007p000A004p000C007 | | 0G007p000U004p000G007p000C0 | |
| | p000A004p000A007p000G004p0 | | 04p000U007p000C004p000C007p | |
| | 00G007p000A004p000G007p000 | | 000U004p000U007p000G004p000 | |
| | C004p000A007p000C004p000C0 | | U007p000A004p000G007p000U00 | |
| | 07p001U004p001U007 | | 4p000G007p000G004p001A004p0 | |
| | | | 01C004 | |
| DT- | C007p001U004p001A007p000C0 | 728 | X033A004p001G007p001A004p00 | 1019 |
| 001674 | 04p000A007p000A004p000G007 | | 0G007p000A004p000G007p000G0 | |
| | p000G004p000A007p000G004p0 | | 04p000U007p000G004p000C007p | |
| | 00C007p000A004p000C007p000 | | 000U004p000C007p000C004p000 | |
| | C004p000U007p000C004p000U0 | | U007p000U004p000G007p000U00 | |
| | 07p001C004p001U007 | | 4p000A007p000G004p001U004p0 | |
| | | | 01G004 | |
| DT- | U007p001A004p001C007p000A0 | 729 | X033A004p001A007p001G004p00 | 1020 |
| 001675 | 04p000A007p000G004p000G007 | | 0A007p000G004p000A007p000G0 | |
| | p000A004p000G007p000C004p0 | | 04p000G007p000U004p000G007p | |
| | 00A007p000C004p000C007p000 | | 000C004p000U007p000C004p000 | |
| | U004p000C007p000U004p000C0 | | C007p000U004p000U007p000G00 | |
| | 07p001U004p001U007 | | 4p000U007p000A004p001G004p0 | |
| | | | 010004 | |
| DT- | C007p001A004p001A007p000G0 | 730 | X033A004p001A007p001G004p00 | 1021 |
| 001676 | 04p000G007p000A004p000G007 | | 0A007p000G004p000A007p000G0 | |
| | p000C004p000A007p000C004p0 | | 04p000A007p000G004p000G007p | |
| | 00C007p000U004p000C007p000 | | 000U004p000G007p000C004p000 | |
| | 0004p000U007p000U004p000U0 | | U007p000U004p000U007p000U00 | |
| | 07p001U004p001U007 | | 4p000U007p000G004001U0040 | |
| | | | 01A004 | |
| DT- | C007p00U0004p001U007p000U0 | 731 | X033U004p001A007p001A004p00 | 1022 |
| 001677 | 04p000c007p000G004p000G007 | | 0G007p000G004p000G007p000A0 | |
| | p000A004p000U007p000A004p0 | | 04p000U007p000G004p000U007p | |
| | 00C007p000A004p000U007p000 | | 000G004p000U007p000C004p000 | |
| | C004p000C007p000C004p000U0 | | C007p000G004p000G007p000A00 | |
| | 07p001U004p001A007 | | 4p000A007p000G004p001U004p0 | |
| | | | 01C004 | |
| DT- | U007p001C004p001C007p000G0 | 732 | X033A004p001A007p001G004p00 | 1023 |
| 001678 | 04p000U007p000A004p000U007 | | 0A007p000A004p000G007p00060 | |
| | p000A004p000U007p000A004p0 | | 04p000G007p000A004p000U007p | |
| | 00U007p000U004p000C007p000 | | 000G004p000U007p000G004p000 | |
| | C004p000U007p000U004p000U0 | | U007p000C004p000C007p000G00 | |
| | 07p001U004p001U007 | | 4p000G007p000A004p001A004p0 | |
| | | | 01G004 | |
| DT- | C007p001C004p001G007p000G0 | 733 | X033U004p001A007p001A004p00 | 1024 |
| 001679 | 04p000A007p000U004p000A007 | | 0G007p000A004p000A007p000G0 | |
| | p000C004p000A007p000U004p0 | | 04p000G007p000G004p000A007p | |
| | 00C007p000C004p000U007p000 | | 000U004p000G007p000U004p000 | |
| | U004p000U007p000C004p000U0 | | G007p000U004p000C007p000U00 | |
| | 07p001U004p001A007 | | 4p000G007p000G004p001A004p0 | |
| | | | 01A004 | |
| DT- | G007p0016004p001A007p000U0 | 734 | X033A004p001A007p001G004p00 | 1025 |
| 001680 | 04p000A007p000C004p000A007 | | 0A007p000A004p0006007p000A0 | |
| | p000U004p000U007p000U004p0 | | 04p000A007p000G004p000G007p | |
| | 00C007p000U004p000U007p000 | | 000G004p000A007p000U004p000 | |
| | C004p000U007p000U004p000U0 | | G007p000U004p000G007p000U00 | |
| | 07p001U004p001U007 | | 4p000C007p000U004p0016004p0 | |
| | | | 01G004 | |
| DT- | G007p001A004p001C007p000A0 | 735 | X033A004p001A007p001A004p00 | 1026 |
| 001681 | 04p000U007p000A004p000U007 | | 0G007p000A004p000A007p000G0 | |
| | p000C004p000C007p000C004p0 | | 04p000A007p000A004p000U007p | |
| | 00U007p000U004p000U007p000 | | 000G004p000G007p000A004p000 | |
| | U004p000U007p000U004p000U0 | | C007p000G004p000U007p000G00 | |
| | 07p001U004p001U007 | | 4p000U007p000C004p001C004p0 | |
| | | | 01G004 | |
| DT- | A007p001C004p001A007p000C0 | 736 | X033U004p001A007p001A004p00 | 1027 |
| 001682 | 04p000A007p000U004p000C007 | | 0A007p000G004p000A007p000A0 | |
| | p000U004p000C007p000U004p0 | | 04p000G007p000A004p000A007p | |
| | 00U007p000C004p000U007p000 | | 000G004p000G007p000G004p000 | |
| | U004p000C007p000U004p000U0 | | A007p000U004p000G007p000U00 | |
| | 07p001U004p001A007 | | 4p000G007p000U004p001C004p0 | |
| | | | 01C004 | |
| DT- | C007p001A004p001C007p000A0 | 737 | X033A004p001C007p001A004p00 | 1028 |
| 001683 | 04p000U007p000C004p000C007 | | 0A007p000A004p000G007p000A0 | |
| | p000C004p000U007p000U004p0 | | 04p000A007p000G004p000A007p | |
| | 00C007p000U004p000U007p000 | | 000A004p000G007p000G004p000 | |
| | C004p000U007p000U004p000U0 | | G007p000A004p000U007p000G00 | |
| | 07p001G004p001U007 | | 4p000U007p000G004p001U004p0 | |
| | | | 01C004 | |
| DT- | U007p001C004p001C007p000C0 | 738 | X033A004p001A007p001G004p00 | 1029 |
| 001684 | 04p000U007p000U004p000C007 | | 0G007p000C004p000C007p000A0 | |
| | p000U004p000U007p000C004p0 | | 04p000A007p000A004p000G007p | |
| | 00U007p000U004p000U007p000 | | 000A004p000A007p000G004p000 | |
| | G004p000G007p000C004p000C0 | | A007p000A004p000G007p000G00 | |
| | 07p001U004p001U007 | | 4p000G007p000A004p001U004p0 | |
| | | | 01G004 | |
| DT- | C007p001C004p001C007p000U0 | 739 | X033A004p001G007p001A004p00 | 1030 |
| 001685 | 04p000U007p000C004p000U007 | | 0G007p000G004p000C007p000C0 | |
| | p000U004p000C007p000U004p0 | | 04p000A007p000A004p000A007p | |
| | 00U007p000U004p000G007p000 | | 000G004p000A007p000A004p000 | |
| | G004p000C007p000C004p000U0 | | G007p000A004p000A007p000G00 | |
| | 07p001C004p001U007 | | 4p000G007p000G004p001A004p0 | |
| | | | 01U004 | |
| DT- | C007p001U004p001U007p000C0 | 740 | X033A004p001U007p001C004p00 | 1031 |
| 001686 | 04p000U007p000U004p000C007 | | 0G007p000A004p000G007p000G0 | |
| | p000U004p000U007p000U004p0 | | 04p000C007p000C004p000A007p | |
| | 00G007p000G004p000C007p000 | | 000A004p000A007p000G004p000 | |
| | C004p000U007p000C004p000G0 | | A007p000A004p000G007p000A00 | |
| | 07p001A004p001U007 | | 4p000A007p000G004p001G004p0 | |
| | | | 01G004 | |
| DT- | U007p001U004p001U007p000G0 | 741 | X033A004p001A007p001U004p00 | 1032 |
| 001687 | 04p000G007p000C004p000C007 | | 0U007p000C004p000C007p000A0 | |
| | p000U004p000C007p000G004p0 | | 04p000G007p000U004p000C007p | |
| | 00A007p000C004p000U007p000 | | 000G004p000A007p000G004p000 | |
| | G004p000G007p000G004p000A0 | | G007p000C004p000C007p000A00 | |
| | 07p001U004p001U007 | | 4p000A007p000A004p001G004p0 | |
| | | | 01A004 | |
| DT- | G007p001C004p001C007p000U0 | 742 | X033A004p001G007p001A004p00 | 1033 |
| 001688 | 04p000C007p000G004p000A007 | | 0C007p000C004p000A007p000U0 | |
| | p000C004p000U007p000G004p0 | | 04p000C007p000C004p000C007p | |
| | 00G007p000G004p000A007p000 | | 000A004p000G007p000U004p000 | |
| | U004p000G007p000G004p000U0 | | C007p000G004p000A007p000G00 | |
| | 07p001C004p001U007 | | 4p000G007p000C004p001C004p0 | |
| | | | 01A004 | |
| DT- | C007p001C004p001U007p000C0 | 743 | X033A004p001A007p001G004p00 | 1034 |
| 001689 | 04p000G007p000A004p000C007 | | 0A007p000C004p000C007p000A0 | |
| | p000U004p000G007p000G004p0 | | 04p000U007p000C004p000C007p | |
| | 00G007p000A004p000U007p000 | | 000C004p000A007p000G004p000 | |
| | G004p000G007p000U004p000C0 | | U007p000C004p000G007p000A00 | |
| | 07p001U004p001U007 | | 4p000G007p000G004p001C004p0 | |
| | | | 01C004 | |
| DT- | C007p001C004p001U007p000U0 | 744 | X033U004p001C007p001G004p00 | 1035 |
| 001690 | 04p000U007p000A004p000C007 | | 0A007p000A004p000A007p000U0 | |
| | p000A004p000C007p000C004p0 | | 04p000C007p000C004p000G007p | |
| | 00G007p000G004p000A007p000 | | 000G004p000U007p000G004p000 | |
| | U004p000U007p000U004p000C0 | | U007p000A004p000A007p000A00 | |
| | 07p001G004p001A007 | | 4p000G007p000G004p001G004p0 | |
| | | | 01U004 | |
| DT- | C007p001U004p001U007p000U0 | 745 | X0330004p001U007p001C004p00 | 1036 |
| 001691 | 04p000A007p000C004p000A007 | | 0G007p000A004p000A007p000A0 | |
| | p000C004p000C007p000G004p0 | | 04p000U007p000C004p000C007p | |
| | 00G007p000A004p000U007p000 | | 000G004p000G007p000U004p000 | |
| | U004p000U007p000C004p000G0 | | G007p000U004p000A007p000A00 | |
| | 07p001A004p001A007 | | 4p000A007p000G004p001G004p0 | |
| | | | 01G004 | |
| DT- | U007p001U004p001U007p000A0 | 746 | X033A004p001U007p001U004p00 | 1037 |
| 001692 | 04p000C007p000A004p000C007 | | 0C007p000G004p000A007p000A0 | |
| | p000C004p000G007p000G004p0 | | 04p000A007p000U004p000C007p | |
| | 00A007p000U004p000U007p000 | | 000C004p000G007p000G004p000 | |
| | U004p000C007p000G004p000A0 | | U007p000G004p000U007p000A00 | |
| | 07p001A004p001U007 | | 4p000A007p000A004p001G004p0 | |
| | | | 01G004 | |
| DT- | U007p001U004p001A007p000C0 | 747 | X033A004p001C007p001U004p00 | 1038 |
| 001693 | 04p000A007p000C004p000C007 | | 0U007p000C004p000G007p000A0 | |
| | p000G004p000G007p000A004p0 | | 04p000A007p000A004p000U007p | |
| | 00U007p000U004p000U007p000 | | 000C004p000C007p000G004p000 | |
| | C004p000G007p000A004p000A0 | | G007p000U004p000G007p000U00 | |
| | 07p001G004p001U007 | | 4p000A007p000A004p001A004p0 | |
| | | | 01G004 | |
| DT- | U007p001A004p001C007p000A0 | 748 | X033A004p001C007p001C004p00 | 1039 |
| 001694 | 04p000C007p000C004p000G007 | | 0U007p000U004p000C007p000G0 | |
| | p000G004p000A007p000U004p0 | | 04p000A007p000A004p000A007p | |
| | 00U007p000U004p000C007p000 | | 0000004p000C007p000C004p000 | |
| | G004p000A007p000A004p000G0 | | G007p000G004p000U007p000G00 | |
| | 07p001G004p001U007 | | 4p000U007p000A004p001A004p0 | |
| | | | 01A004 | |
| DT- | A007p001C004p001A007p000C0 | 749 | X033A004p001A007p001C004p00 | 1040 |
| 001695 | 04p000C007p000G004p000G007 | | 0C007p000U004p000U007p000C0 | |
| | p000A004p000U007p000U004p0 | | 04p000G007p000A004p000A007p | |
| | 00U007p000C004p000G007p000 | | 000A004p000U007p000C004p000 | |
| | A004p000A007p000G004p000G0 | | C007p000G004p000G007p000U00 | |
| | 07p001U004p001U007 | | 4p000G007p000U004p001A004p0 | |
| | | | 01A004 | |
| DT- | C007p001G004p001G007p000A0 | 750 | X033A004p001U007p001G004p00 | 1041 |
| 001696 | 04p000U007p000U004p000U007 | | 0G007p000C004p000A007p000C0 | |
| | p000C004p000G007p000A004p0 | | 04p000C007p000U004p000U007p | |
| | 00A007p000G004p000G007p000 | | 000C004p000G007p000A004p000 | |
| | 0004p000G007p000C004p000C0 | | A007p000A004p000U007p000C00 | |
| | 07p001A004p001U007 | | 4p000C007p000G004p001G004p0 | |
| | | | 01U004 | |
| DT- | U007p001C004p001G007p000A0 | 751 | X033A004p001U007p001G004p00 | 1042 |
| 001697 | 04p000A007p000G004p000G007 | | 0U007p000C004p000G007p000G0 | |
| | p000U004p000G007p000C004p0 | | 04p000U007p000G004p000G007p | |
| | 00C007p000A004p000C007p000 | | 000C004p000A007p000C004p000 | |
| | C004p000G007p000A004p000C0 | | C007p000U004p000U007p000C00 | |
| | 07p001A004p001U007 | | 4p000G007p000A004p001A004p0 | |
| | | | 01A004 | |
| DT- | G007p001A004p001A007p000G0 | 752 | X033A004p001U007p001G004p00 | 1043 |
| 001698 | 04p000G007p000U004p000G007 | | 0U007p000G004p000U007p000C0 | |
| | p000C004p000C007p000A004p0 | | 04p000G007p000G004p000U007p | |
| | 00C007p000C004p000G007p000 | | 000G004p000G007p000C004p000 | |
| | A004p000C007p000A004p000C0 | | A007p000C004p000C007p000U00 | |
| | 07p001A004p001U007 | | 4p000U007p000C004p001G004p0 | |
| | | | 01A004 | |
| DT- | A007p001A004p001G007p000G0 | 753 | X033A004p001A007p001U004p00 | 1044 |
| 001699 | 04p000U007p000G004p000C007 | | 0G007p000U004p000G007p000U0 | |
| | p000C004p000A007p000C004p0 | | 04p000C007p000G004p000G007p | |
| | 00C007p000G004p000A007p000 | | 0000004p000G007p000G004p000 | |
| | C004p000A007p000C004p000A0 | | C007p000A004p000C007p000C00 | |
| | 07p001U004p001U007 | | 4p000U007p000U004p001C004p0 | |
| | | | 01G004 | |
| DT- | G007p001U004p001G007p000C0 | 754 | X0330004p001U007p001G004p00 | 1045 |
| 001700 | 04p000C007p000A004p000C007 | | 0C007p000A004p000U007p000G0 | |
| | p000C004p000G007p000A004p0 | | 04p000U007p000G004p000U007p | |
| | 00C007p000A004p000C007p000 | | 000C004p000G007p000G004p000 | |
| | A004p000U007p000G004p000C0 | | U007p000G004p000G007p000C00 | |
| | 07p001A004p001A007 | | 4p000A007p000C004p001C004p0 | |
| | | | 010004 | |
| DT- | U007p001G004p001C007p000C0 | 755 | X033A004p001U007p001U004p00 | 1046 |
| 001701 | 04p000A007p000C004p000C007 | | 0G007p000C004p000A007p000U0 | |
| | p000G004p000A007p000C004p0 | | 04p000G007p000U004p000G007p | |
| | 00A007p000C004p000A007p000 | | 0000004p000C007p000G004p000 | |
| | U004p000G007p000C004p000A0 | | G007p000U004p000G007p000G00 | |
| | 07p001A004p001U007 | | 4p000C007p000A004p001C004p0 | |
| | | | 01C004 | |
| DT- | C007p001C004p001A007p000C0 | 756 | X033A004p001A007p001G004p00 | 1047 |
| 001702 | 04p000C007p000G004p000A007 | | 0U007p000U004p000G007p000C0 | |
| | p000C004p000A007p000C004p0 | | 04p000A007p000U004p000G007p | |
| | 00A007p000U004p000G007p000 | | 0000004p000G007p000U004p000 | |
| | C004p000A007p000A004p000C0 | | C007p000G004p000G007p000U00 | |
| | 07p001U004p001U007 | | 4p000G007p000G004p001C004p0 | |
| | | | 01A004 | |
| DT- | C007p001A004p001C007p000C0 | 757 | X033A004p001A007p001A004p00 | 1048 |
| 001703 | 04p000G007p000A004p000C007 | | 0G007p000U004p000U007p000G0 | |
| | p000A004p000C007p000A004p0 | | 04p000C007p000A004p000U007p | |
| | 00U007p000G004p000C007p000 | | 000G004p000U007p000G004p000 | |
| | A004p000A007p000C004p000U0 | | U007p000C004p000G007p000G00 | |
| | 07p001U004p001U007 | | 4p000U007p000G004p001G004p0 | |
| | | | 01C004 | |
| DT- | C007p001G004p001A007p000C0 | 758 | X033A004p001U007p001C004p00 | 1049 |
| 001704 | 04p000A007p000C004p000A007 | | 0G007p000A004p000A007p000G0 | |
| | p000U004p000G007p000C004p0 | | 04p000U007p000U004p000G007p | |
| | 00A007p000A004p000C007p000 | | 000C004p000A007p000U004p000 | |
| | U004p000U007p000C004p000G0 | | G007p000U004p000G007p000U00 | |
| | 07p001A004p001U007 | | 4p000C007p000G004p001G004p0 | |
| | | | 01U004 | |
| DT- | G007p001A004p001C007p000A0 | 759 | X033A004p001G007p001U004p00 | 1050 |
| 001705 | 04p000C007p000A004p000U007 | | 0C007p000G004p000A007p000A0 | |
| | p000G004p000C007p000A004p0 | | 04p000G007p000U004p000U007p | |
| | 00A007p000C004p000U007p000 | | 000G004p000C007p000A004p000 | |
| | U004p000C007p000G004p000A0 | | U007p000G004p000U007p000G00 | |
| | 07p001C004p001U007 | | 4p000U007p000C004p001G004p0 | |
| | | | 01G004 | |
| DT- | A007p001C004p001A007p000C0 | 760 | X033A004p001A007p001G004p00 | 1051 |
| 001706 | 04p000A007p000U004p000G007 | | 0U007p000C004p000G007p000A0 | |
| | p000C004p000A007p000A004p0 | | 04p000A007p000G004p000U007p | |
| | 00C007p000U004p000U007p000 | | 0000004p000G007p000C004p000 | |
| | C004p000G007p000A004p000C0 | | A007p000U004p000G007p000U00 | |
| | 07p001U004p001U007 | | 4p000G007p000U004p001C004p0 | |
| | | | 01G004 | |
| DT- | C007p001A004p001C007p000A0 | 761 | X0330004p001A007p001A004p00 | 1052 |
| 001707 | 04p000U007p000G004p000C007 | | 0G007p000U004p000C007p000G0 | |
| | p000A004p000A007p000C004p0 | | 04p000A007p000A004p000G007p | |
| | 00U007p000U004p000C007p000 | | 0000004p000U007p000G004p000 | |
| | G004p000A007p000C004p000U0 | | C007p000A004p000U007p000G00 | |
| | 07p001U004p001A007 | | 4p000U007p000G004p001U004p0 | |
| | | | 01C004 | |
| DT- | A007p001C004p001A007p000U0 | 762 | X033A004p001C007p001A004p00 | 1053 |
| 001708 | 04p000G007p000C004p000A007 | | 0A007p000G004p000U007p000C0 | |
| | p000A004p000C007p000U004p0 | | 04p000G007p000A004p000A007p | |
| | 00U007p000C004p000G007p000 | | 000G004p000U007p000U004p000 | |
| | A004p000C007p000U004p000U0 | | G007p000C004p000A007p000U00 | |
| | 07p001G004p001U007 | | 4p000G007p000U004p001G004p0 | |
| | | | 01U004 | |
| DT- | A007p001U004p001G007p000C0 | 763 | X033A004p001A007p001C004p00 | 1054 |
| 001709 | 04p000A007p000A004p000C007 | | 0U007p000A004p000A007p000G0 | |
| | p000U004p000U007p000C004p0 | | 04p000U007p000C004p000G007p | |
| | 00G007p000A004p000C007p000 | | 000A004p000A007p000G004p000 | |
| | U004p000U007p000G004p000G0 | | U007p000U004p000G007p000C00 | |
| | 07p001U004p001U007 | | 4p000A007p000U004p001G004p0 | |
| | | | 010004 | |
| DT- | C007p001A004p001A007p000C0 | 764 | X033A004p001U007p001C004p00 | 1055 |
| 001710 | 04p000U007p000U004p000C007 | | 0C007p000A004p000C007p000C0 | |
| | p000G004p000A007p000C004p0 | | 04p000A007p000A004p000G007p | |
| | 00U007p000U004p000G007p000 | | 0000004p000C007p000G004p000 | |
| | G004p000U007p000G004p000G0 | | A007p000A004p000G007p000U00 | |
| | 07p001A004p001U007 | | 4p000U007p000G004p001C004p0 | |
| | | | 01A004 | |
| DT- | C007p001U004p001U007p000C0 | 765 | X033G004p001U007p001C004p00 | 1056 |
| 001711 | 04p000G007p000A004p000C007 | | 0C007p000U004p000C007p000C0 | |
| | p000U004p000U007p000G004p0 | | 04p000A007p000C004p000C007p | |
| | 00G007p000U004p000G007p000 | | 000A004p000A007p000G004p000 | |
| | G004p000A007p000G004p000G0 | | U007p000C004p000G007p000A00 | |
| | 07p001A004p001C007 | | 4p000A007p000G004p001U004p0 | |
| | | | 01U004 | |
| DT- | C007p001U004p001G007p000U0 | 766 | X033A004p001U007p001U004p00 | 1057 |
| 001712 | 04p000C007p000G004p000G007 | | 0C007p000C004p000C007p000G0 | |
| | p000A004p000C007p000A004p0 | | 04p000A007p000A004p000U007p | |
| | G004p000G007p000G004p000A0 | | 000G004p000U007p000C004p000 | |
| | 07p001A004p001U007 | | C007p000G004p000A007p000C00 | |
| | | | 4p000A007p000G004p001U004p0 | |
| | | | 01G004 | |
| DT- | C007p001G004p001G007p000A0 | 767 | X033G004p001C007p001A004p00 | 1058 |
| 001713 | 04p000C007p000A004p000U007 | | 0C007p000C004p000U007p000U0 | |
| | p000U004p000C007p000G004p0 | | 04p000C007p000C004p000C007p | |
| | 00G007p000G004p000A007p000 | | 000G004p000A007p000A004p000 | |
| | A004p000G007p000G004p000U0 | | U007p000G004p000U007p000C00 | |
| | 07p001G004p001C007 | | 4p000C007p000G004p001A004p0 | |
| | | | 01C004 | |
| DT- | G007p001C004p001U007p000A0 | 768 | X033A004p001U007p001G004p00 | 1059 |
| 001714 | 04p000C007p000U004p000C007 | | 0C007p000A004p000G007p000G0 | |
| | p000C004p000U007p000A004p0 | | 04p000A007p000G004p000U007p | |
| | 00C007p000U004p000C007p000 | | 000A004p000G007p000G004p000 | |
| | C004p000U007p000G004p000C0 | | A007p000G004p000U007p000A00 | |
| | 07p001A004p001U007 | | 4p000G007p000C004p001C004p0 | |
| | | | 01C004 | |
| DT- | C007p001U004p001A007p000C0 | 769 | X033A004p001A007p001U004p00 | 1060 |
| 001715 | 04p000U007p000C004p000C007 | | 0G007p000C004p000A007p000G0 | |
| | p000U004p000A007p000C004p0 | | 04p000G007p000A004p000G007p | |
| | 00U007p000C004p000C007p000 | | 0000004p000A007p000G004p000 | |
| | U004p000G007p000C004p000A0 | | G007p000A004p000G007p000U00 | |
| | 07p001U004p001U007 | | 4p000A007p000G004p001C004p0 | |
| | | | 01C004 | |
| DT- | C007p001A004p001C007p000A0 | 770 | X033A004p001U007p001C004p00 | 1061 |
| 001716 | 04p000C007p000C004p000C007 | | 0C007p000A004p000G007p000U0 | |
| | p000A004p000U007p000G004p0 | | 04p000U007p000C004p000C007p | |
| | 00G007p000A004p000A007p000 | | 000A004p000U007p000G004p000 | |
| | C004p000U007p000G004p000G0 | | G007p000G004p000U007p000G00 | |
| | 07p001A004p001U007 | | 4p000U007p000G004p001G004p0 | |
| | | | 01G004 | |
| DT- | C007p001A004p001U007p000G0 | 771 | X033A004p001U007p001C004p00 | 1062 |
| 001717 | 04p000G007p000A004p000A007 | | 0G007p000G004p000C007p000C0 | |
| | p000C004p000U007p000G004p0 | | 04p000U007p000C004p000C007p | |
| | 00G007p000A004p000G007p000 | | 000A004p000G007p000U004p000 | |
| | G004p000C007p000C004p000G0 | | U007p000C004p000C007p000A00 | |
| | 07p001A004p001U007 | | 4p000U007p000G004p001G004p0 | |
| | | | 01G004 | |
| DT- | G007p001C004p001A007p000G0 | 772 | X033A004p001U007p001G004p00 | 1063 |
| 001718 | 04p000C007p000U004p000G007 | | 0U007p000G004p000G007p000C0 | |
| | p000C004p000U007p000U004p0 | | 04p000U007p000C004p000A007p | |
| | 00G007p000A004p000G007p000 | | 000A004p000G007p000C004p000 | |
| | C004p000C007p000A004p000C0 | | A007p000G004p000C007p000U00 | |
| | 07p001A004p001U007 | | 4p000G007p000C004p001U004p0 | |
| | | | 01C004 | |
| DT- | G007p001C004p001U007p000G0 | 773 | X033A004p001A007p001C004p00 | 1064 |
| 001719 | 04p000C007p000U004p000U007 | | 0G007p000U004p000G007p000U0 | |
| | p000G004p000A007p000G004p0 | | 04p000G007p000G004p000C007p | |
| | 00C007p000C004p000A007p000 | | 000U004p000C007p000A004p000 | |
| | C004p000A007p000C004p000G0 | | A007p000G004p000C007p000A00 | |
| | 07p001U004p001U007 | | 4p000G007p000C004p001U004p0 | |
| | | | 01G004 | |
| DT- | C007p001U004p001U007p000G0 | 774 | X033A004p001U007p001U004p00 | 1065 |
| 001720 | 04p000A007p000G004p000C007 | | 0G007p000C004p000A007p000C0 | |
| | p000C004p000A007p000C004p0 | | 04p000G007p000U004p000G007p | |
| | 00A007p000C004p000G007p000 | | 000U004p000G007p000G004p000 | |
| | U004p000G007p000C004p000A0 | | C007p000U004p000C007p000A00 | |
| | 07p001A004p001U007 | | 4p000A007p000G004p001C004p0 | |
| | | | 01A004 | |
| DT- | G007p001G004p001A007p000U0 | 775 | X033A004p001A007p001C004p00 | 1066 |
| 001721 | 04p000G007p000A004p000A007 | | 0U007p000U004p000C007p000A0 | |
| | p000A004p000C007p000A004p0 | | 04p000G007p000C004p000U007p | |
| | 00G007p000C004p000U007p000 | | 000G004p000U007p000U004p000 | |
| | G004p000A007p000A004p000G0 | | U007p000C004p000A007p000U00 | |
| | 07p001U004p001U007 | | 4p000C007p000C004p001U004p0 | |
| | | | 01G004 | |
| DT- | G007p001A004p001U007p000G0 | 776 | X033A004p001C007p001A004p00 | 1067 |
| 001722 | 04p000A007p000A004p000A007 | | 0C007p000U004p000U007p000C0 | |
| | p000C004p000A007p000G004p0 | | 04p000A007p000G004p000C007p | |
| | 00C007p000U004p000G007p000 | | 0000004p000G007p000U004p000 | |
| | A004p000A007p000G004p000U0 | | U007p000U004p000C007p000A00 | |
| | 07p001G004p001U007 | | 4p000U007p000C004p001C004p0 | |
| | | | 01U004 | |
| DT- | G007p001A004p001A007p000A0 | 777 | X033A004p001U007p001G004p00 | 1068 |
| 001723 | 04p000C007p000A004p000G007 | | 0C007p000C004p000A007p000C0 | |
| | p000C004p000U007p000G004p0 | | 04p000U007p000U004p000C007p | |
| | 00A007p000A004p000G007p000 | | 000A004p000G007p000C004p000 | |
| | U004p000G007p000G004p000C0 | | U007p000G004p000U007p000U00 | |
| | 07p001A004p001U007 | | 4p000U007p000C004p001A004p0 | |
| | | | 01U004 | |
| DT- | A007p001A004p001C007p000A0 | 778 | X033A004p001A007p001C004p00 | 1069 |
| 001724 | 04p000G007p000C004p000U007 | | 0U007p000G004p000C007p000C0 | |
| | p000G004p000A007p000A004p0 | | 04p000A007p000C004p000U007p | |
| | 00G007p000U004p000G007p000 | | 0000004p000C007p000A004p000 | |
| | G004p000C007p000A004p000G0 | | G007p000C004p000U007p000G00 | |
| | 07p001U004p001U007 | | 4p000U007p000U004p001U004p0 | |
| | | | 01C004 | |
| DT- | G007p001C004p001U007p000G0 | 779 | X033A004p001U007p001G004p00 | 1070 |
| 001725 | 04p000A007p000A004p000G007 | | 0G007p000A004p000A007p000C0 | |
| | p000U004p000G007p000G004p0 | | 04p000U007p000G004p000C007p | |
| | 00C007p000A004p000G007p000 | | 000C004p000A007p000C004p000 | |
| | U004p000U007p000C004p000C0 | | U007p000U004p000C007p000A00 | |
| | 07p001A004p001U007 | | 4p000G007p000C004p001U004p0 | |
| | | | 01G004 | |
| DT- | A007p001G004p001U007p000C0 | 780 | X033A004p001U007p001G004p00 | 1071 |
| 001726 | 04p000A007p000A004p000C007 | | 0C007p000C004p000U007p000C0 | |
| | p000U004p000A007p000C004p0 | | 04p000G007p000C004p000G007p | |
| | 00G007p000C004p000G007p000 | | 0000004p000A007p000G004p000 | |
| | A004p000G007p000G004p000C0 | | U007p000U004p000G007p000A00 | |
| | 07p001A004p001U007 | | 4p000C007p000U004p001G004p0 | |
| | | | 01G004 | |
| DT- | U007p001C004p001A007p000A0 | 781 | X033A004p001U007p001C004p00 | 1072 |
| 001727 | 04p000C007p000U004p000A007 | | 0U007p000G004p000C007p000C0 | |
| | p000C004p000G007p000C004p0 | | 04p000U007p000C004p000G007p | |
| | 00G007p000A004p000G007p000 | | 000C004p000G007p000U004p000 | |
| | G004p000C007p000A004p000G0 | | A007p000G004p000U007p000U00 | |
| | 07p001A004p001U007 | | 4p000G007p000A004p001C004p0 | |
| | | | 01U004 | |
| DT- | C007p001U004p001A007p000C0 | 782 | X033A004p001A007p001G004p00 | 1073 |
| 001728 | 04p000G007p000C004p000G007 | | 0C007p000C004p000U007p000C0 | |
| | p000A004p000G007p000G004p0 | | 04p000U007p000G004p000C007p | |
| | 00C007p000A004p000G007p000 | | 000C004p000U007p000C004p000 | |
| | A004p000G007p000G004p000C0 | | G007p000C004p000G007p000U00 | |
| | 07p001U004p001U007 | | 4p000A007p000G004p001U004p0 | |
| | | | 01U004 | |
| DT- | C007p001G004p001A007p000G0 | 783 | X033G004p001U007p001U004p00 | 1074 |
| 001729 | 04p000G007p000C004p000A007 | | 0C007p000C004p000G007p000A0 | |
| | p000G004p000A007p000G004p0 | | 04p000G007p000C004p000C007p | |
| | 00G007p000C004p000U007p000 | | 0000004p000C007p000U004p000 | |
| | C004p000G007p000G004p000A0 | | G007p000C004p000C007p000U00 | |
| | 07p001A004p001C007 | | 4p000C007p000G004p001C004p0 | |
| | | | 01G004 | |
| DT- | G007p001G004p001C007p000U0 | 784 | X0330004p001A007p001G004p00 | 1075 |
| 001730 | 04p000C007p000G004p000G007 | | 0G007p000U004p000C007p000C0 | |
| | p000A004p000A007p000C004p0 | | 04p000C007p000G004p000G007p | |
| | 00C007p000G004p000G007p000 | | 0000004p000U007p000C004p000 | |
| | G004p000A007p000C004p000C0 | | C007p000G004p000A007p000G00 | |
| | 07p001U004p001A007 | | 4p000C007p000C004p001U004p0 | |
| | | | 01C004 | |
| DT- | G007p001C004p001U007p000C0 | 785 | X033A004p001U007p001A004p00 | 1076 |
| 001731 | 04p000G007p000G004p000A007 | | 0G007p000G004p000U007p000C0 | |
| | p000A004p000C007p000C004p0 | | 04p000C007p000C004p000G007p | |
| | 00G007p000G004p000G007p000 | | 000G004p000U007p000U004p000 | |
| | A004p000C007p000C004p000U0 | | C007p000C004p000G007p000A00 | |
| | 07p001A004p001U007 | | 4p000G007p000C004p001C004p0 | |
| | | | 01U004 | |
| DT- | U007p001C004p001G007p000G0 | 786 | X033A004p001U007p001C004p00 | 1077 |
| 001732 | 04p000A007p000A004p000C007 | | 0U007p000A004p000G007p000G0 | |
| | p000C004p000G007p000G004p0 | | 04p000U007p000C004p000C007p | |
| | 00G007p000A004p000C007p000 | | 000C004p000G007p000G004p000 | |
| | C004p000U007p000A004p000G0 | | U007p000U004p000C007p000C00 | |
| | 07p001A004p001U007 | | 4p000G007p000A004p001G004p0 | |
| | | | 01C004 | |
| DT- | G007p001C004p001A007p000G0 | 787 | X033A004p001A007p001U004p00 | 1078 |
| 001733 | 04p000U007p000U004p000G007 | | 0C007p000C004p000G007p000C0 | |
| | p000C004p000A007p000G004p0 | | 04p000U007p000C004p000C007p | |
| | 00G007p000A004p000G007p000 | | 0000004p000G007p000C004p000 | |
| | C004p000G007p000G004p000A0 | | A007p000A004p000C007p000U00 | |
| | 07p001U004p001U007 | | 4p000G007p000C004p001C004p0 | |
| | | | 01G004 | |
| DT- | U007p001G004p001C007p000A0 | 788 | X033A004p001A007p001C004p00 | 1079 |
| 001734 | 04p000G007p000G004p000A007 | | 0U007p000C004p000C007p000A0 | |
| | p000G004p000C007p000G004p0 | | 04p000U007p000C004p000C007p | |
| | 00G007p000A004p000U007p000 | | 000G004p000C007p000U004p000 | |
| | G004p000G007p000A004p000G0 | | C007p000C004p000U007p000G00 | |
| | 07p001U004p001U007 | | 4p000C007p000A004p001A004p0 | |
| | | | 01C004 | |
| DT- | G007p001C004p001A007p000G0 | 789 | X0330004p001A007p001A004p00 | 1080 |
| 001735 | 04p000G007p000A004p000G007 | | 0C007p000U004p000C007p000C0 | |
| | p000C004p000G007p000G004p0 | | 04p000A007p000U004p000C007p | |
| | 00A007p000U004p000G007p000 | | 000C004p000G007p000C004p000 | |
| | G004p000A007p000G004p000U0 | | U007p000C004p000C007p000U00 | |
| | 07p001U004p001A007 | | 4p000G007p000C004p001A004p0 | |
| | | | 01A004 | |
| DT- | G007p001G004p001A007p000G0 | 790 | X033A004p001A007p001G004p00 | 1081 |
| 001736 | 04p000C007p000G004p000G007 | | 0C007p000A004p000A007p000C0 | |
| | p000A004p000U007p000G004p0 | | 04p000U007p000C004p000C007p | |
| | 00G007p000A004p000G007p000 | | 000A004p000U007p000C004p000 | |
| | U004p000U007p000G004p000C0 | | C007p000G004p000C007p000U00 | |
| | 07p001U004p001U007 | | 4p000C007p000C004p001U004p0 | |
| | | | 01G004 | |
| DT- | U007p001A004p001G007p000G0 | 791 | X033A004p001U007p001C004p00 | 1082 |
| 001737 | 04p000C007p000C004p000U007 | | 0C007p000G004p000A007p000U0 | |
| | p000G004p000G007p000C004p0 | | 04p000A007p000G004p000G007p | |
| | 00C007p000U004p000A007p000 | | 000C004p000C007p000A004p000 | |
| | U004p000C007p000G004p000G0 | | G007p000G004p000C007p000C00 | |
| | 07p001A004p001U007 | | 4p000U007p000A004p001G004p0 | |
| | | | 01G004 | |
| DT- | G007p001G004p001C007p000C0 | 792 | X033A004p001A007p001A004p00 | 1083 |
| 001738 | 04p000U007p000A004p000U007 | | 0G007p000C004p000G007p000C0 | |
| | p000C004p000G007p000G004p0 | | 04p000C007p000U004p000C007p | |
| | 00A007p000G004p000G007p000 | | 000C004p000G007p000A004p000 | |
| | C004p000G007p000C004p000U0 | | U007p000A004p000G007p000G00 | |
| | 07p001U004p001U007 | | 4p000C007p000C004p001A004p0 | |
| | | | 01G004 | |
| DT- | G007p001C004p001C007p000U0 | 793 | X0330004p001A007p001A004p00 | 1084 |
| 001739 | 04p000A007p000U004p000C007 | | 0A007p000G004p000C007p000G0 | |
| | p000G004p000G007p000A004p0 | | 04p000C007p000C004p000U007p | |
| | 00G007p000G004p000C007p000 | | 000C004p000C007p000G004p000 | |
| | G004p000C007p000U004p000U0 | | A007p000U004p000A007p000G00 | |
| | 07p001U004p001A007 | | 4p000G007p000C004p001C004p0 | |
| | | | 01A004 | |
| DT- | C007p001G004p001C007p000U0 | 794 | X033A004p001A007p001C004p00 | 1085 |
| 001740 | 04p000U007p000U004p000C007 | | 0A007p000G004p000G007p000A0 | |
| | p000C004p000C007p000U004p0 | | 04p000G007p000C004p000A007p | |
| | 00G007p000C004p000U007p000 | | 000G004p000G007p000G004p000 | |
| | C004p000C007p000U004p000G0 | | A007p000A004p000A007p000G00 | |
| | 07p001U004p001U007 | | 4p000C007p000G004p001C004p0 | |
| | | | 01C004 | |
| DT- | G007p001C004p001U007p000U0 | 795 | X0330004p001A007p001A004p00 | 1086 |
| 001741 | 04p000U007p000C004p000C007 | | 0C007p000A004p000G007p000G0 | |
| | p000C004p000U007p000G004p0 | | 04p000A007p000G004p000C007p | |
| | 00C007p000U004p000C007p000 | | 000A004p000G007p000G004p000 | |
| | C004p000U007p000G004p000U0 | | G007p000A004p000A007p000A00 | |
| | 07p001U004p001A007 | | 4p000G007p000C004p001G004p0 | |
| | | | 01C004 | |
| DT- | C007p001U004p001G007p000C0 | 796 | X0330004p001A007p001A004p00 | 1087 |
| 001742 | 04p000U007p000C004p000C007 | | 0C007p000G004p000G007p000C0 | |
| | p000U004p000G007p000U004p0 | | 04p000G007p000A004p000A007p | |
| | 00U007p000C004p000G007p000 | | 000C004p000A007p000G004p000 | |
| | C004p000C007p000G004p000U0 | | G007p000A004p000G007p000C00 | |
| | 07p001U004p001A007 | | 4p000A007p000G004p001G004p0 | |
| | | | 01G004 | |
| DT- | C007p001G004p001G007p000C0 | 797 | X033A004p001A007p001C004p00 | 1088 |
| 001743 | 04p000C007p000A004p000A007 | | 0U007p000G004p000C007p000G0 | |
| | p000C004p000U007p000C004p0 | | 04p000G007p000U004p000G007p | |
| | 00A007p000C004p000C007p000 | | 000A004p000G007p000U004p000 | |
| | G004p000C007p000A004p000G0 | | U007p000G004p000G007p000C00 | |
| | 07p001U004p001U007 | | 4p000C007p000G004p001G004p0 | |
| | | | 01C004 | |
| DT- | G007p001C004p001C007p000A0 | 798 | X033A004p001A007p001G004p00 | 1089 |
| 001744 | 04p000A007p000C004p000U007 | | 0A007p000C004p000U007p000G0 | |
| | p000C004p000A007p000C004p0 | | 04p000C007p000G004p000G007p | |
| | 00C007p000G004p000C007p000 | | 0000004p000G007p000A004p000 | |
| | A004p000G007p000U004p000C0 | | G007p000U004p000U007p000G00 | |
| | 07p001U004p001U007 | | 4p000G007p000C004p001C004p0 | |
| | | | 01G004 | |
| DT- | C007p001G004p001C007p000U0 | 799 | X033U004p001U007p001C004p00 | 1090 |
| 001745 | 04p000C007p000C004p000C007 | | 0U007p000A004p000G007p000G0 | |
| | p000U004p000G007p000A004p0 | | 04p000G007p000U004p000U007p | |
| | 00A007p000C004p000C007p000 | | 000C004p000A007p000G004p000 | |
| | C004p000U007p000A004p000G0 | | G007p000G004p000A007p000G00 | |
| | 07p001A004p001A007 | | 4p000C007p000G004p001C004p0 | |
| | | | 01G004 | |
| DT- | G007p001C004p001U007p000C0 | 800 | X033A004p001U007p001U004p00 | 1091 |
| 001746 | 04p000C007p000C004p000U007 | | 0C007p000U004p000A007p000G0 | |
| | p000G004p000A007p000A004p0 | | 04p000G007p000G004p000U007p | |
| | 00C007p000C004p000C007p000 | | 0000004p000C007p000A004p000 | |
| | 0004p000A007p000G004p000A0 | | G007p000G004p000G007p000A00 | |
| | 07p001A004p001U007 | | 4p000G007p000C004p001G004p0 | |
| | | | 01C004 | |
| DT- | C007p001C004p001U007p000G0 | 801 | X033G004p001A007p001C004p00 | 1092 |
| 001747 | 04p000A007p000A004p000C007 | | 0A007p000G004p000U007p000U0 | |
| | p000C004p000C007p000U004p0 | | 04p000C007p000U004p000A007p | |
| | 00A007p000G004p000A007p000 | | 000G004p000G007p000G004p000 | |
| | A004p000C007p000U004p000G0 | | U007p000U004p000C007p000A00 | |
| | 07p001U004p001C007 | | 4p000G007p000G004p001G004p0 | |
| | | | 01A004 | |
| DT- | C007p001U004p001G007p000A0 | 802 | X033A004p001G007p001A004p00 | 1093 |
| 001748 | 04p000A007p000C004p000C007 | | 0C007p000A004p000G007p000U0 | |
| | p000C004p000U007p000A004p0 | | 04p000U007p000C004p000U007p | |
| | 00G007p000A004p000A007p000 | | 000A004p000G007p000G004p000 | |
| | C004p000U007p000G004p000U0 | | G007p000U004p000U007p000C00 | |
| | 07p001C004p001U007 | | 4p000A007p000G004p001G004p0 | |
| | | | 01G004 | |
| DT- | U007p001G004p001A007p000A0 | 803 | X033A004p001A007p001G004p00 | 1094 |
| 001749 | 04p000C007p000C004p000C007 | | 0A007p000C004p000A007p000G0 | |
| | p000U004p000A007p000G004p0 | | 04p000U007p000U004p000C007p | |
| | 00A007p000A004p000C007p000 | | 0000004p000A007p000G004p000 | |
| | U004p000G007p000U004p000C0 | | G007p000G004p000U007p000U00 | |
| | 07p001U004p001U007 | | 4p000C007p000A004p001G004p0 | |
| | | | 01G004 | |
| DT- | G007p001A004p001A007p000C0 | 804 | X033A004p001A007p001A004p00 | 1095 |
| 001750 | 04p000C007p000C004p000U007 | | 0G007p000A004p000C007p000A0 | |
| | p000A004p000G007p000A004p0 | | 04p000G007p000U004p000U007p | |
| | 00A007p000C004p000U007p000 | | 000C004p000U007p000A004p000 | |
| | G004p000U007p000C004p000U0 | | G007p000G004p000G007p000U00 | |
| | 07p001U004p001U007 | | 4p000U007p000C004p001A004p0 | |
| | | | 01G004 | |
| DT- | A007p001A004p001C007p000C0 | 805 | X033A004p001G007p001A004p00 | 1096 |
| 001751 | 04p000C007p000U004p000A007 | | 0A007p000G004p000A007p000C0 | |
| | p000G004p000A007p000A004p0 | | 04p000A007p000G004p000U007p | |
| | 00C007p000U004p000G007p000 | | 0000004p000C007p000U004p000 | |
| | U004p000C007p000U004p000U0 | | A007p000G004p000G007p000G00 | |
| | 07p001C004p001U007 | | 4p000U007p000U004p001C004p0 | |
| | | | 01A004 | |
| DT- | A007p001C004p001C007p000C0 | 806 | X0330004p001C007p001G004p00 | 1097 |
| 001752 | 04p000U007p000A004p000G007 | | 0A007p000A004p000G007p000A0 | |
| | p000A004p000A007p000C004p0 | | 04p000C007p000A004p000G007p | |
| | 00U007p000G004p000U007p000 | | 0000004p000U007p000C004p000 | |
| | C004p000U007p000U004p000C0 | | U007p000A004p000G007p000G00 | |
| | 07p001G004p001A007 | | 4p000G007p000U004p001U004p0 | |
| | | | 01C004 | |
| DT- | C007p001C004p001C007p000U0 | 807 | X033A004p001U007p001C004p00 | 1098 |
| 001753 | 04p000A007p000G004p000A007 | | 0G007p000A004p000A007p000G0 | |
| | p000A004p000C007p000U004p0 | | 04p000A007p000C004p000A007p | |
| | 00G007p000U004p000C007p000 | | 000G004p000U007p000U004p000 | |
| | U004p000U007p000C004p000G0 | | C007p000U004p000A007p000G00 | |
| | 07p001A004p001U007 | | 4p000G007p000G004p001U004p0 | |
| | | | 01U004 | |
| DT- | C007p001C004p001U007p000A0 | 808 | X033A004p001G007p001U004p00 | 1099 |
| 001754 | 04p000G007p000A004p000A007 | | 0C007p000G004p000A007p000A0 | |
| | p000C004p000U007p000G004p0 | | 04p000G007p000A004p000C007p | |
| | 00U007p000C004p000U007p000 | | 000A004p000G007p000U004p000 | |
| | U004p000C007p000G004p000A0 | | U007p000C004p000U007p000A00 | |
| | 07p001C004p001U007 | | 4p000G007p000G004p001G004p0 | |
| | | | 01U004 | |
| DT- | C007p001U004p001A007p000G0 | 809 | X033A004p001A007p001G004p00 | 1100 |
| 001755 | 04p000A007p000A004p000C007 | | 0U007p000C004p000G007p000A0 | |
| | p000U004p000G007p000U004p0 | | 04p000A007p000G004p000A007p | |
| | 00C007p000U004p000U007p000 | | 000C004p000A007p000G004p000 | |
| | C004p000G007p000A004p000C0 | | U007p000U004p000C007p000U00 | |
| | 07p001U004p001U007 | | 4p000A007p000G004p001G004p0 | |
| | | | 01G004 | |
| DT- | C007p001C004p001A007p000G0 | 810 | X033A004p001U007p001C004p00 | 1101 |
| 001756 | 04p000C007p000U004p000C007 | | 0A007p000C004p000A007p000G0 | |
| | p000C004p000A007p000G004p0 | | 04p000G007p000A004p000C007p | |
| | 00U007p000C004p000C007p000 | | 0000004p000G007p000G004p000 | |
| | U004p000G007p000U004p000G0 | | A007p000G004p000C007p000U00 | |
| | 07p001A004p001U007 | | 4p000G007p000G004p001G004p0 | |
| | | | 01C004 | |
| DT- | C007p001A004p001G007p000C0 | 811 | X033A004p001A007p001U004p00 | 1102 |
| 001757 | 04p000U007p000C004p000C007 | | 0C007p000A004p000C007p000A0 | |
| | p000A004p000G007p000U004p0 | | 04p000G007p000G004p000A007p | |
| | 00C007p000C004p000U007p000 | | 000C004p000U007p000G004p000 | |
| | G004p000U007p000G004p000A0 | | G007p000A004p000G007p000C00 | |
| | 07p001U004p001U007 | | 4p000U007p000G004p001G004p0 | |
| | | | 01G004 | |
| DT- | C007p001U004p001U007p000G0 | 812 | X033A004p001A007p001G004p00 | 1103 |
| 001758 | 04p000U007p000A004p000G007 | | 0C007p000A004p000U007p000U0 | |
| | p000C004p000C007p000G004p0 | | 04p000C007p000C004p000C007p | |
| | 00G007p000G004p000A007p000 | | 000G004p000G007p000C004p000 | |
| | A004p000U007p000G004p000C0 | | U007p000A004p000C007p000A00 | |
| | 07p001U004p001U007 | | 4p000A007p000G004p001G004p0 | |
| | | | 01A004 | |
| DT- | U007p001A004p001G007p000C0 | 813 | X033A004p001C007p001A004p00 | 1104 |
| 001759 | 04p000C007p000G004p000G007 | | 0G007p000C004p000A007p000G0 | |
| | p000G004p000A007p000A004p0 | | 04p000C007p000A004p000U007p | |
| | 00U007p000G004p000C007p000 | | 0000004p000C007p000C004p000 | |
| | 0004p000G007p000C004p000U0 | | C007p000G004p000G007p000C00 | |
| | 07p001G004p001U007 | | 4p000U007p000A004p001C004p0 | |
| | | | 01A004 | |
| DT- | U007p001U004p001C007p000U0 | 814 | X033U004p001U007p001C004p00 | 1105 |
| 001760 | 04p000U007p000U004p000C007 | | 0A007p000G004p000C007p000C0 | |
| | p000G004p000G007p000C004p0 | | 04p000U007p000G004p000G007p | |
| | 00C007p000A004p000G007p000 | | 000C004p000C007p000G004p000 | |
| | G004p000C007p000U004p000G0 | | A007p000A004p000A007p000G00 | |
| | 07p001A004p001A007 | | 4p000A007p000A004p001A004p0 | |
| | | | 01G004 | |
| DT- | C007p001U004p001G007p000A0 | 815 | X033A004p001U007p001C004p00 | 1106 |
| 001761 | 04p000G007p000G004p000C007 | | 0C007p000A004p000C007p000G0 | |
| | p000C004p000C007p000U004p0 | | 04p000U007p000C004p000A007p | |
| | 00G007p000A004p000C007p000 | | 000G004p000G007p000G004p000 | |
| | G004p000U007p000G004p000G0 | | C007p000C004p000U007p000C00 | |
| | 07p001A004p001U007 | | 4p000A007p000G004p001C004p0 | |
| | | | 01C004 | |
| DT- | U007p001G004p001A007p000G0 | 816 | X033A004p001A007p001U004p00 | 1107 |
| 001762 | 04p000G007p000C004p000C007 | | 0C007p000C004p000A007p000C0 | |
| | p000C004p000U007p000G004p0 | | 04p000G007p000U004p000C007p | |
| | 00A007p000C004p000G007p000 | | 000A004p000G007p000G004p000 | |
| | 0004p000G007p000G004p000A0 | | G007p000C004p000C007p000U00 | |
| | 07p001U004p001U007 | | 4p000C007p000A004p001G004p0 | |
| | | | 01C004 | |
| DT- | C007p001C004p001C007p000U0 | 817 | X033U004p001U007p001G004p00 | 1108 |
| 001763 | 04p000G007p000A004p000C007 | | 0C007p000C004p000C007p000A0 | |
| | p000G004p000U007p000G004p0 | | 04p000U007p000C004p000C007p | |
| | 00G007p000A004p000U007p000 | | 000A004p000C007p000G004p000 | |
| | G004p000G007p000G004p000C0 | | U007p000C004p000A007p000G00 | |
| | 07p001A004p001A007 | | 4p000G007p000G004p001C004p0 | |
| | | | 01C004 | |
| DT- | C007p001C004p001U007p000G0 | 818 | X033U004p001U007p001U004p00 | 1109 |
| 001764 | 04p000A007p000C004p000G007 | | 0G007p000C004p000C007p000C0 | |
| | p000U004p000G007p000G004p0 | | 04p000A007p000U004p000C007p | |
| | 00A007p000U004p000G007p000 | | 000C004p000A007p000C004p000 | |
| | G004p000G007p000C004p000A0 | | G007p000U004p000C007p000A00 | |
| | 07p001A004p001A007 | | 4p000G007p000G004p001G004p0 | |
| | | | 01C004 | |
| DT- | C007p001U004p001G007p000A0 | 819 | X033A004p001U007p001U004p00 | 1110 |
| 001765 | 04p000C007p000G004p000U007 | | 0U007p000G004p000C007p000C0 | |
| | p000G004p000G007p000A004p0 | | 04p000C007p000A004p000U007p | |
| | 00U007p000G004p000G007p000 | | 000C004p000C007p000A004p000 | |
| | G004p000C007p000A004p000A0 | | C007p000G004p000U007p000C00 | |
| | 07p001A004p001U007 | | 4p000A007p000G004p001G004p0 | |
| | | | 01G004 | |
| DT- | U007p001G004p001U007p000G0 | 820 | X033G004p001A007p001G004p00 | 1111 |
| 001766 | 04p000C007p000A004p000U007 | | 0C007p000A004p000G007p000G0 | |
| | p000G004p000A007p000C004p0 | | 04p000G007p000C004p000G007p | |
| | 00G007p000C004p000C007p000 | | 0000004p000C007p000A004p000 | |
| | C004p000U007p000G004p000C0 | | U007p000G004p000C007p000A00 | |
| | 07p001U004p001C007 | | 4p000C007p000A004p001A004p0 | |
| | | | 01G004 | |
| DT- | G007p001U004p001G007p000C0 | 821 | X033A004p001G007p001A004p00 | 1112 |
| 001767 | 04p000A007p000U004p000G007 | | 0G007p000C004p000A007p000G0 | |
| | p000A004p000C007p000G004p0 | | 04p000G007p000G004p000C007p | |
| | 00C007p000C004p000C007p000 | | 000G004p000U007p000C004p000 | |
| | U004p000G007p000C004p000U0 | | A007p000U004p000G007p000C00 | |
| | 07p001C004p001U007 | | 4p000A007p000C004p001A004p0 | |
| | | | 01A004 | |
| DT- | G007p001C004p001U007p000U0 | 822 | X033U004p001C007p001A004p00 | 1113 |
| 001768 | 04p000A007p000C004p000U007 | | 0A007p000A004p000U007p000U0 | |
| | p000C004p000G007p000G004p0 | | 04p000U007p000C004p000C007p | |
| | 00G007p000A004p000A007p000 | | 000C004p000G007p000A004p000 | |
| | A004p000U007p000U004p000U0 | | G007p000U004p000A007p000A00 | |
| | 07p001G004p001A007 | | 4p000G007p000C004p001A004p0 | |
| | | | 01G004 | |
| DT- | C007p001U004p001U007p000A0 | 823 | X033A004p001G007p001C004p00 | 1114 |
| 001769 | 04p000C007p000U004p000C007 | | 0A007p000A004p000A007p000U0 | |
| | p000G004p000G007p000G004p0 | | 04p000U007p000U004p000C007p | |
| | 00A007p000A004p000A007p000 | | 000C004p000C007p000G004p000 | |
| | U004p000U007p000U004p000G0 | | A007p000G004p000U007p000A00 | |
| | 07p001C004p001U007 | | 4p000A007p000G004p001C004p0 | |
| | | | 01A004 | |
| DT- | U007p001U004p001A007p000C0 | 824 | X033A004p001A007p001G004p00 | 1115 |
| 001770 | 04p000U007p000C004p000G007 | | 0C007p000A004p000A007p000A0 | |
| | p000G004p000G007p000A004p0 | | 04p000U007p000U004p000U007p | |
| | 00A007p000A004p000U007p000 | | 000C004p000C007p000C004p000 | |
| | U004p000U007p000G004p000C0 | | G007p000A004p000G007p000U00 | |
| | 07p001U004p001U007 | | 4p000A007p000A004p001G004p0 | |
| | | | 01C004 | |
| DT- | U007p001A004p001C007p000U0 | 825 | X033A004p001A007p001A004p00 | 1116 |
| 001771 | 04p000C007p000G004p000G007 | | 0G007p000C004p000A007p000A0 | |
| | p000G004p000A007p000A004p0 | | 04p000A007p000U004p000U007p | |
| | 00A007p000U004p000U007p000 | | 0000004p000C007p000C004p000 | |
| | 0004p000G007p000C004p000U0 | | C007p000G004p000A007p000G00 | |
| | 07p001U004p001U007 | | 4p000U007p000A004p001A004p0 | |
| | | | 01G004 | |
| DT- | A007p001C004p001U007p000C0 | 826 | X033A004p001A007p001A004p00 | 1117 |
| 001772 | 04p000G007p000G004p000G007 | | 0A007p000G004p000C007p000A0 | |
| | p000A004p000A007p000A004p0 | | 04p000A007p000A004p000U007p | |
| | 00U007p000U004p000U007p000 | | 0000004p000U007p000C004p000 | |
| | G004p000C007p000U004p000U0 | | C007p000C004p000G007p000A00 | |
| | 07p001U004p001U007 | | 4p000G007p000U004p001A004p0 | |
| | | | 01A004 | |
| DT- | C007p001U004p001C007p000G0 | 827 | X033U004p001A007p001A004p00 | 1118 |
| 001773 | 04p000G007p000G004p000A007 | | 0A007p000A004p000G007p000C0 | |
| | p000A004p000A007p000U004p0 | | 04p000A007p000A004p000A007p | |
| | 00U007p000U004p000G007p000 | | 0000004p000U007p000U004p000 | |
| | C004p000U007p000U004p000U0 | | C007p000C004p000C007p000G00 | |
| | 07p001U004p001A007 | | 4p000A007p000G004p001U004p0 | |
| | | | 01A004 | |
| DT- | U007p001C004p001G007p000G0 | 828 | X033U004p001C007p001A004p00 | 1119 |
| 001774 | 04p000G007p000A004p000A007 | | 0A007p000A004p000A007p000G0 | |
| | p000A004p000U007p000U004p0 | | 04p000C007p000A004p000A007p | |
| | 00U007p000G004p000C007p000 | | 000A004p000U007p000U004p000 | |
| | U004p000U007p000U004p000U0 | | U007p000C004p000C007p000C00 | |
| | 07p001G004p001U007 | | 4p000G007p000A004p001G004p0 | |
| | | | 01U004 | |
| DT- | G007p001G004p001A007p000A0 | 829 | X033U004p001U007p001G004p00 | 1120 |
| 001775 | 04p000A007p000U004p000U007 | | 0G007p000C004p000A007p000A0 | |
| | p000U004p000G007p000C004p0 | | 04p000A007p000A004p000G007p | |
| | U004p000G007p000C004p000C0 | | 000C004p000A007p000A004p000 | |
| | 07p001A004p001A007 | | A007p000U004p000U007p000U00 | |
| | | | 4p000C007p000C004p001C004p0 | |
| | | | 01G004 | |
| DT- | G007p001A004p001A007p000A0 | 830 | X033U004p001U007p001U004p00 | 1121 |
| 001776 | 04p000U007p000U004p000U007 | | 0G007p000G004p000C007p000A0 | |
| | p000G004p000C007p000U004p0 | | 04p000A007p000A004p000A007p | |
| | 00U007p000U004p000U007p000 | | 000G004p000C007p000A004p000 | |
| | G004p000C007p000C004p000A0 | | A007p000A004p000U007p000U00 | |
| | 07p001A004p001A007 | | 4p000U007p000C004p001C004p0 | |
| | | | 01C004 | |
| DT- | A007p001A004p001A007p000U0 | 831 | X033A004p001U007p001U004p00 | 1122 |
| 001777 | 04p000U007p000U004p000G007 | | 0U007p000G004p000G007p000C0 | |
| | p000C004p000U007p000U004p0 | | 04p000A007p000A004p000A007p | |
| | 00U007p000U004p000G007p000 | | 000A004p000G007p000C004p000 | |
| | C004p000C007p000A004p000A0 | | A007p000A004p000A007p000U00 | |
| | 07p001A004p001U007 | | 4p000U007p000U004p001C004p0 | |
| | | | 01C004 | |
| DT- | G007p001C004p001U007p000U0 | 832 | X033A004p001A007p001G004p00 | 1123 |
| 001778 | 04p000U007p000U004p000G007 | | 0C007p000G004p000G007p000G0 | |
| | p000C004p000C007p000A004p0 | | 04p000U007p000U004p000U007p | |
| | 00A007p000A004p000C007p000 | | 000G004p000G007p000C004p000 | |
| | C004p000C007p000G004p000C0 | | A007p000A004p000A007p000A00 | |
| | 07p001U004p001U007 | | 4p000G007p000C004p001A004p0 | |
| | | | 01A004 | |
| DT- | G007p001C004p001U007p000U0 | 833 | X033A004p001U007p001A004p00 | 1124 |
| 001779 | 04p000C007p000G004p000G007 | | 0U007p000C004p000C007p000A0 | |
| | p000U004p000G007p000G004p0 | | 04p000A007p000A004p000C007p | |
| | 00U007p000U004p000U0070p00 | | 000C004p000G007p000C004p000 | |
| | G004p000G007p000A004p000U0 | | C007p000G004p000A007p000A00 | |
| | 07p001A004p001U007 | | 4p000G007p000C004p001G004p0 | |
| | | | 01G004 | |
| DT- | C007p001U004p001U007p000C0 | 834 | X033A004p001A007p001U004p00 | 1125 |
| 001780 | 04p000G007p000G004p000C007 | | 0A007p000U004p000C007p000C0 | |
| | p000G004p000G007p000U004p0 | | 04p000A007p000A004p000A007p | |
| | 00U007p000U004p000G007p000 | | 000C004p000C007p000G004p000 | |
| | G004p000A007p000U004p000A0 | | C007p000C004p000G007p000A00 | |
| | 07p001U004p001U007 | | 4p000A007p000G004p001C004p0 | |
| | | | 01G004 | |
| DT- | U007p001U004p001C007p000G0 | 835 | X033A004p001A007p001A004p00 | 1126 |
| 001781 | 04p000G007p000C004p000G007 | | 0U007p000A004p000U007p000C0 | |
| | p000G004p000U007p000U004p0 | | 04p000C007p000A004p000A007p | |
| | 00U007p000G004p000G007p000 | | 000A004p000C007p000C004p000 | |
| | A004p000U007p000A004p000U0 | | G007p000C004p000C007p000G00 | |
| | 07p001U004p001U007 | | 4p000A007p000A004p001G004p0 | |
| | | | 01C004 | |
| DT- | U007p001C004p001G007p000G0 | 836 | X0330004p001A007p001A004p00 | 1127 |
| 001782 | 04p000C007p000G004p000G007 | | 0A007p000U004p000A007p000U0 | |
| | p000U004p000U007p000U004p0 | | 04p000C007p000C004p000A007p | |
| | 00G007p000G004p000A007p000 | | 000A004p000A007p000C004p000 | |
| | U004p000A007p000U004p000U0 | | C007p000G004p000C007p000C00 | |
| | 07p001U004p001A007 | | 4p000G007p000A004p001A004p0 | |
| | | | 01G004 | |
| DT- | C007p001G004p001G007p000C0 | 837 | X033A004p001U007p001A004p00 | 1128 |
| 001783 | 04p000G007p000G004p000U007 | | 0A007p000A004p000U007p000A0 | |
| | p000U004p000U007p000G004p0 | | 04p000U007p000C004p000C007p | |
| | 00G007p000A004p000U007p000 | | 000A004p000A007p000A004p000 | |
| | A004p000U007p000U004p000U0 | | C007p000C004p000G007p000C00 | |
| | 07p001A004p001U007 | | 4p000C007p000G004p001A004p0 | |
| | | | 01A004 | |
| DT- | G007p001G004p001C007p000G0 | 838 | X033A004p001A007p001U004p00 | 1129 |
| 001784 | 04p000G007p000U004p000U007 | | 0A007p000A004p000A007p000U0 | |
| | p000U004p000G007p000G004p0 | | 04p000A007p000U004p000C007p | |
| | 00A007p000U004p000A007p000 | | 000C004p000A007p000A004p000 | |
| | U004p000U007p000U004p000A0 | | A007p000C004p000C007p000G00 | |
| | 07p001U004p001U007 | | 4p000C007p000C004p001G004p0 | |
| | | | 01A004 | |
| DT- | G007p001C004p001G007p000G0 | 839 | X033A004p001A007p001A004p00 | 1130 |
| 001785 | 04p000U007p000U004p000U007 | | 0U007p000A004p000A007p000A0 | |
| | p000G004p000G007p000A004p0 | | 04p000U007p000A004p000U007p | |
| | 00U007p000A004p000U007p000 | | 000C004p000C007p000A004p000 | |
| | U004p000U007p000A004p000U0 | | A007p000A004p000C007p000C00 | |
| | 07p001U004p001U007 | | 4p000G007p000C004p001C004p0 | |
| | | | 01G004 | |
| DT- | C007p001G004p001G007p000U0 | 840 | X033U004p001C007p001A004p00 | 1131 |
| 001786 | 04p000U007p000U004p000G007 | | 0A007p000U004p000A007p000A0 | |
| | p000G004p000A007p000U004p0 | | 04p000A007p000U004p000A007p | |
| | 00A007p000U004p000U007p000 | | 0000004p000C007p000C004p000 | |
| | U004p000A007p000U004p000U0 | | A007p000A004p000A007p000C00 | |
| | 07p001G004p001A007 | | 4p000C007p000G004p001C004p0 | |
| | | | 01C004 | |
| DT- | G007p001G004p001U007p000U0 | 841 | X033A004p001U007p001C004p00 | 1132 |
| 001787 | 04p000U007p000G004p000G007 | | 0A007p000A004p000U007p000A0 | |
| | p000A004p000U007p000A004p0 | | 04p000A007p000A004p000U007p | |
| | A004p000U007p000U004p000G0 | | 000A004p000U007p000C004p000 | |
| | 07p001A004p001U007 | | C007p000A004p000A007p000A00 | |
| | | | 4p000C007p000C004p001G004p0 | |
| | | | 01C004 | |
| DT- | G007p001U004p001U007p000U0 | 842 | X033A004p001G007p001U004p00 | 1133 |
| 001788 | 04p000G007p000G004p000A007 | | 0C007p000A004p000A007p000U0 | |
| | p000U004p000A007p000U004p0 | | 04p000A007p000A004p000A007p | |
| | 00U007p000U004p000A007p000 | | 0000004p000A007p000U004p000 | |
| | U004p000U007p000G004p000A0 | | C007p000C004p000A007p000A00 | |
| | 07p001C004p001U007 | | 4p000A007p000C004p001C004p0 | |
| | | | 01G004 | |
| DT- | U007p001U004p001U007p000G0 | 843 | X033A004p001G007p001G004p00 | 1134 |
| 001789 | 04p000G007p000A004p000U007 | | 0U007p000C004p000A007p000A0 | |
| | p000A004p000U007p000U004p0 | | 04p000U007p000A004p000A007p | |
| | 00U007p000A004p000U007p000 | | 000A004p000U007p000A004p000 | |
| | U004p000G007p000A004p000C0 | | U007p000C004p000C007p000A00 | |
| | 07p001C004p001U007 | | 4p000A007p000A004p001C004p0 | |
| | | | 01C004 | |
| DT- | U007p001U004p001G007p000G0 | 844 | X033A004p001A007p001G004p00 | 1135 |
| 001790 | 04p000A007p000U004p000A007 | | 0G007p000U004p000C007p000A0 | |
| | p000U004p000U007p000U004p0 | | 04p000A007p000U004p000A007p | |
| | 00A007p000U004p000U007p000 | | 000A004p000A007p000U004p000 | |
| | G004p000A007p000C004p000C0 | | A007p000U004p000C007p000C00 | |
| | 07p001U004p001U007 | | 4p000A007p000A004p001A004p0 | |
| | | | 01C004 | |
| DT- | U007p001G004p001G007p000A0 | 845 | X033A004p001G007p001A004p00 | 1136 |
| 001791 | 04p000U007p000A004p000U007 | | 0G007p000G004p000U007p000C0 | |
| | p000U004p000U007p000A004p0 | | 04p000A007p000A004p000U007p | |
| | 00U007p000U004p000G007p000 | | 000A004p000A007p000A004p000 | |
| | A004p000C007p000C004p000U0 | | U007p000A004p000U007p000C00 | |
| | 07p001C004p001U007 | | 4p000C007p000A004p001A004p0 | |
| | | | 01A004 | |
| DT- | G007p001G004p001A007p000U0 | 846 | X033A004p001C007p001G004p00 | 1137 |
| 001792 | 04p000A007p000U004p000U007 | | 0A007p000G004p000G007p000U0 | |
| | p000U004p000A007p000U004p0 | | 04p000C007p000A004p000A007p | |
| | 00U007p000G004p000A007p000 | | 0000004p000A007p000A004p000 | |
| | C004p000C007p000U004p000C0 | | A007p000U004p000A007p000U00 | |
| | 07p001G004p001U007 | | 4p000C007p000C004p001A004p0 | |
| | | | 01A004 | |
| DT- | G007p001A004p001U007p000A0 | 847 | X0330004p001A007p001C004p00 | 1138 |
| 001793 | 04p000U007p000U004p000U007 | | 0G007p000A004p000G007p000G0 | |
| | p000A004p000U007p000U004p0 | | 04p000U007p000C004p000A007p | |
| | 00G007p000A004p000C007p000 | | 000A004p000U007p000A004p000 | |
| | C004p000U007p000C004p000G0 | | A007p000A004p000U007p000A00 | |
| | 07p001U004p001A007 | | 4p000U007p000C004p001C004p0 | |
| | | | 01A004 | |
| DT- | A007p001U004p001A007p000U0 | 848 | X033A004p001G007p001A004p00 | 1139 |
| 001794 | 04p000U007p000U004p000A007 | | 0C007p000G004p000A007p000G0 | |
| | p000U004p000U007p000G004p0 | | 04p000G007p000U004p000C007p | |
| | 00A007p000C004p000C007p000 | | 000A004p000A007p000U004p000 | |
| | 0004p000C007p000G004p000U0 | | A007p000A004p000A007p000U00 | |
| | 07p001C004p001U007 | | 4p000A007p000U004p001C004p0 | |
| | | | 01C004 | |
| DT- | U007p001U004p001U007p000A0 | 849 | X0330004p001G007p001A004p00 | 1140 |
| 001795 | 04p000U007p000U004p000G007 | | 0G007p000G004p000A007p000C0 | |
| | p000A004p000C007p000C004p0 | | 04p000G007p000A004p000G007p | |
| | 00U007p000C004p000G007p000 | | 000G004p000U007p000C004p000 | |
| | U004p000C007p000C004p000U0 | | A007p000A004p000U007p000A00 | |
| | 07p001C004p001A007 | | 4p000A007p000A004p001U004p0 | |
| | | | 01A004 | |
| DT- | A007p001U004p001U007p000G0 | 850 | X033A004p001U007p001C004p00 | 1141 |
| 001796 | 04p000A007p000C004p000C007 | | 0G007p000G004p000A007p000G0 | |
| | p000U004p000C007p000G004p0 | | 04p000G007p000A004p000C007p | |
| | 00U007p000C004p000C007p000 | | 000G004p000A007p000G004p000 | |
| | U004p000C007p000C004p000G0 | | G007p000U004p000C007p000A00 | |
| | 07p001A004p001U007 | | 4p000A007p000U004p001A004p0 | |
| | | | 01A004 | |
| DT- | U007p001C004p001G007p000U0 | 851 | X0330004p001C007p001A004p00 | 1142 |
| 001797 | 04p000C007p000C004p000U007 | | 0G007p000C004p000G007p000A0 | |
| | p000C004p000C007p000G004p0 | | 04p000G007p000U004p000C007p | |
| | 00A007p000C004p000U007p000 | | 000G004p000G007p000A004p000 | |
| | C004p000G007p000C004p000U0 | | G007p000G004p000A007p000C00 | |
| | 07p001G004p001A007 | | 4p000G007p000A004p001G004p0 | |
| | | | 01G004 | |
| DT- | U007p001C004p001C007p000U0 | 852 | X033A004p001U007p001G004p00 | 1143 |
| 001798 | 04p000C007p000C004p000G007 | | 0U007p000C004p000A007p000G0 | |
| | p000A004p000C007p000U004p0 | | 04p000C007p000G004p000A007p | |
| | 00C007p000G004p000C007p000 | | 000G004p000U007p000C004p000 | |
| | U004p000G007p000A004p000C0 | | G007p000G004p000A007p000G00 | |
| | 07p001A004p001U007 | | 4p000G007p000A004p001C004p0 | |
| | | | 01G004 | |
| DT- | C007p001C004p001C007p000A0 | 853 | X033A004p001U007p001C004p00 | 1144 |
| 001799 | 04p000A007p000U004p000C007 | | 0C007p000A004p000A007p000A0 | |
| | p000C004p000A007p000C004p0 | | 04p000A007p000C004p000G007p | |
| | 00G007p000U004p000U007p000 | | 0000004p000G007p000G004p000 | |
| | U004p000U007p000G004p000G0 | | A007p000U004p000U007p000G00 | |
| | 07p001A004p001U007 | | 4p000G007p000G004p001U004p0 | |
| | | | 01G004 | |
| DT- | C007p001C004p001A007p000A0 | 854 | X033A004p001A007p001U004p00 | 1145 |
| 001800 | 04p000U007p000C004p000C007 | | 0C007p000C004p000A007p000A0 | |
| | p000A004p000C007p000G004p0 | | 04p000A007p000A004p000C007p | |
| | 00U007p000U004p000U007p000 | | 000G004p000U007p000G004p000 | |
| | U004p000G007p000G004p000A0 | | G007p000A004p000U007p000U00 | |
| | 07p001U004p001U007 | | 4p000G007p000G004p001G004p0 | |
| | | | 01U004 | |
| DT- | C007p001A004p001A007p000U0 | 855 | X0330004p001C007p001A004p00 | 1146 |
| 001801 | 04p000C007p000C004p000A007 | | 0U007p000C004p000C007p000A0 | |
| | p000C004p000G007p000U004p0 | | 04p000A007p000A004p000A007p | |
| | 00U007p000U004p000U007p000 | | 000C004p000G007p000U004p000 | |
| | G004p000G007p000A004p000U0 | | G007p000G004p000A007p000U00 | |
| | 07p001G004p001A007 | | 4p000U007p000G004p001G004p0 | |
| | | | 01G004 | |
| DT- | A007p001A004p001U007p000C0 | 856 | X0330004p001G007p001C004p00 | 1147 |
| 001802 | 04p000C007p000A004p000C007 | | 0A007p000U004p000C007p000C0 | |
| | p000G004p000U007p000U004p0 | | 04p000A007p000A004p000A007p | |
| | 00U007p000U004p000G007p000 | | 000A004p000C007p000G004p000 | |
| | G004p000A007p000U004p000G0 | | U007p000G004p000G007p000A00 | |
| | 07p001C004p001A007 | | 4p000U007p000U004p001G004p0 | |
| | | | 01G004 | |
| DT- | A007p001U004p001C007p000C0 | 857 | X033A004p001U007p001G004p00 | 1148 |
| 001803 | 04p000A007p000C004p000G007 | | 0C007p000A004p000U007p000C0 | |
| | p000U004p000U007p000U004p0 | | 04p000C007p000A004p000A007p | |
| | 00U007p000G004p000G007p000 | | 000A004p000A007p000C004p000 | |
| | A004p000U007p000G004p000C0 | | G007p000U004p000G007p000G00 | |
| | 07p001A004p001U007 | | 4p000A007p000U004p001U004p0 | |
| | | | 01G004 | |
| DT- | C007p001C004p001A007p000C0 | 858 | X033A004p001A007p001G004p00 | 1149 |
| 001804 | 04p000G007p000U004p000U007 | | 0U007p000G004p000C007p000A0 | |
| | p000U004p000U007p000G004p0 | | 04p000U007p000C004p000C007p | |
| | 00G007p000A004p000U007p000 | | 000A004p000A007p000A004p000 | |
| | G004p000C007p000A004p000C0 | | A007p000C004p000G007p000U00 | |
| | 07p001U004p001U007 | | 4p000G007p000G004p001A004p0 | |
| | | | 01U004 | |
| DT- | C007p001A004p001C007p000G0 | 859 | X0330004p001C007p001A004p00 | 1150 |
| 001805 | 04p000U007p000U004p000U007 | | 0G007p000U004p000G007p000C0 | |
| | p000U004p000G007p000G004p0 | | 04p000A007p000U004p000C007p | |
| | 00A007p000U004p000G007p000 | | 000C004p000A007p000A004p000 | |
| | C004p000A007p000C004p000U0 | | A007p000A004p000C007p000G00 | |
| | 07p001G004p001A007 | | 4p000U007p000G004p001G004p0 | |
| | | | 01A004 | |
| DT- | A007p001C004p001G007p000U0 | 860 | X033A004p001U007p001C004p00 | 1151 |
| 001806 | 04p000U007p000U004p000U007 | | 0A007p000G004p000U007p000G0 | |
| | p000G004p000G007p000A004p0 | | 04p000C007p000A004p000U007p | |
| | 00U007p000G004p000C007p000 | | 000C004p000C007p000A004p000 | |
| | A004p000C007p000U004p000G0 | | A007p000A004p000A007p000C00 | |
| | 07p001A004p001U007 | | 4p000G007p000U004p001G004p0 | |
| | | | 01G004 | |
| DT- | C007p001G004p001U007p000U0 | 861 | X0330004p001C007p001U004p00 | 1152 |
| 001807 | 04p000U007p000U004p000G007 | | 0C007p000A004p000G007p000U0 | |
| | p000G004p000A007p000U004p0 | | 04p000G007p000C004p000A007p | |
| | 00G007p000C004p000A007p000 | | 000U004p000C007p000C004p000 | |
| | C004p000U007p000G004p000A0 | | A007p000A004p000A007p000A00 | |
| | 07p001G004p001A007 | | 4p000C007p000G004p001U004p0 | |
| | | | 01G004 | |
| DT- | G007p001U004p001U007p000U0 | 862 | X033A004p001U007p001C004p00 | 1153 |
| 001808 | 04p000U007p000G004p000G007 | | QU007p000C004p000A007p000G0 | |
| | p000A004p000U007p000G004p0 | | 04p000U007p000G004p000C007p | |
| | 00C007p000A004p000C007p000 | | 000A004p000U007p000C004p000 | |
| | 0004p000G007p000A004p000G0 | | C007p000A004p000A007p000A00 | |
| | 07p001A004p001U007 | | 4p000A007p000C004p001G004p0 | |
| | | | 01U004 | |
| DT- | U007p001U004p001U007p000U0 | 863 | X033A004p001G007p001U004p00 | 1154 |
| 001809 | 04p000G007p000G004p000A007 | | 0C007p000U004p000C007p000A0 | |
| | p000U004p000G007p000C004p0 | | 04p000G007p000U004p000G007p | |
| | 00A007p000C004p000U007p000 | | 000C004p000A007p000U004p000 | |
| | G004p000A007p000G004p000A0 | | C007p000C004p000A007p000A00 | |
| | 07p001C004p001U007 | | 4p000A007p000A004p001C004p0 | |
| | | | 01G004 | |
| DT- | C007p001C004p001U007p000C0 | 864 | X033U1027p001A007p001G004p0 | 1155 |
| 000841 | 04p000G007p000G004p000U007 | | 00A007p000C004p000A007p000A | |
| | p000A004p000U007p000U004p0 | | 004p000U007p000A004p000A007 | |
| | 00U007p000A004p000U007p000 | | p000A004p000U007p000A004p00 | |
| | U004p000G007p000U004p000C0 | | 0C007p000C004p000G007p000A0 | |
| | 07p001U004p001A007 | | 04p000G007p000G004p001A004p | |
| | | | 001A004 | |
| DT- | C007p001C004p001C007p000G0 | 865 | X0330004p001U007p001U004p00 | 1156 |
| 001810 | 04p000A007p000C004p000C007 | | 0A007p000U004p000U007p000C0 | |
| | p000C004p000U007p000C004p0 | | 04p000G007p000C004p000G007p | |
| | 00G007p000C004p000G007p000 | | 000A004p000G007p000G004p000 | |
| | A004p000A007p000U004p000A0 | | G007p000U004p000C007p000G00 | |
| | 07p001A004p001A007 | | 4p000G007p000G004p001U004p0 | |
| | | | 01G004 | |
| DT- | C007p001C004p001G007p000A0 | 866 | X0330004p001U007p001U004p00 | 1157 |
| 001811 | 04p000C007p000C004p000C007 | | 0U007p000A004p000U007p000U0 | |
| | p000U004p000C007p000G004p0 | | 04p000C007p000G004p000C007p | |
| | 00C007p000G004p000A007p000 | | 000G004p000A007p000G004p000 | |
| | A004p000U007p000A004p000A0 | | G007p000G004p000U007p000C00 | |
| | 07p001A004p001A007 | | 4p000G007p000G004p001G004p0 | |
| | | | 01U004 | |
| DT- | C007p001G004p001A007p000C0 | 867 | X033A004p001U007p001U004p00 | 1158 |
| 001812 | 04p000C007p000C004p000U007 | | 0U007p000U004p000A007p000U0 | |
| | p000C004p000G007p000C004p0 | | 04p000U007p000C004p000G007p | |
| | 00G007p000A004p000A007p000 | | 000C004p000G007p000A004p000 | |
| | 0004p000A007p000A004p000A0 | | G007p000G004p000G007p000U00 | |
| | 07p001A004p001U007 | | 4p000C007p000G004p001G004p0 | |
| | | | 01G004 | |
| DT- | G007p001A004p001C007p000C0 | 868 | X033A004p001C007p001U004p00 | 1159 |
| 001813 | 04p000C007p000U004p000C007 | | 0U007p000U004p000U007p000A0 | |
| | p000G004p000C007p000G004p0 | | 04p000U007p000U004p000C007p | |
| | 00A007p000A004p000U007p000 | | 000G004p000C007p000G004p000 | |
| | A004p000A007p000A004p000A0 | | A007p000G004p000G007p000G00 | |
| | 07p001G004p001U007 | | 4p000U007p000C004p001G004p0 | |
| | | | 01G004 | |
| DT- | A007p001C004p001C007p000C0 | 869 | X033A004p001C007p001C004p00 | 1160 |
| 001814 | 04p000U007p000C004p000G007 | | 0U007p000U004p000U007p000U0 | |
| | p000C004p000G007p000A004p0 | | 04p000A007p000U004p000U007p | |
| | 00A007p000U004p000A007p000 | | 000C004p000G007p000C004p000 | |
| | A004p000A007p000A004p000G0 | | G007p000A004p000G007p000G00 | |
| | 07p001G004p001U007 | | 4p000G007p000U004p001C004p0 | |
| | | | 01G004 | |
| DT- | C007p001C004p001C007p000U0 | 870 | X033A004p001G007p001C004p00 | 1161 |
| 001815 | 04p000C007p000G004p000C007 | | 0C007p000U004p000U007p000U0 | |
| | p000G004p000A007p000A004p0 | | 04p000U007p000A004p000U007p | |
| | 00U007p000A004p000A007p000 | | 000U004p000C007p000G004p000 | |
| | A004p000A007p000G004p000G0 | | C007p000G004p000A007p000G00 | |
| | 07p001C004p001U007 | | 4p000G007p000G004p001U004p0 | |
| | | | 01C004 | |
| DT- | U007p001C004p001G007p000C0 | 871 | X0330004p001A007p001G004p00 | 1162 |
| 001816 | 04p000G007p000A004p000A007 | | 0G007p000G004p000C007p000C0 | |
| | p000U004p000A007p000A004p0 | | 04p000U007p000U004p000U007p | |
| | 00A007p000A004p000G007p000 | | 0000004p000A007p000U004p000 | |
| | G004p000C007p000C004p000C0 | | U007p000C004p000G007p000C00 | |
| | 07p001U004p001A007 | | 4p000G007p000A004p001G004p0 | |
| | | | 01G004 | |
| DT- | C007p001G004p001C007p000G0 | 872 | X0330004p001G007p001A004p00 | 1163 |
| 001817 | 04p000A007p000A004p000U007 | | 0G007p000G004p000G007p000C0 | |
| | p000A004p000A007p000A004p0 | | 04p000C007p000U004p000U007p | |
| | 00A007p000G004p000G007p000 | | 0000004p000U007p000A004p000 | |
| | C004p000C007p000C004p000U0 | | U007p000U004p000C007p000G00 | |
| | 07p001C004p001A007 | | 4p000C007p000G004p001A004p0 | |
| | | | 01G004 | |
| DT- | G007p001C004p001G007p000A0 | 873 | X033U004p001G007p001G004p00 | 1164 |
| 001818 | 04p000A007p000U004p000A007 | | 0A007p000G004p000G007p000G0 | |
| | p000A004p000A007p000A004p0 | | 04p000C007p000C004p000U007p | |
| | 00G007p000G004p000C007p000 | | 0000004p000U007p000U004p000 | |
| | C004p000C007p000U004p000C0 | | A007p000U004p000U007p000C00 | |
| | 07p001C004p001A007 | | 4p000G007p000C004p001G004p0 | |
| | | | 01A004 | |
| DT- | C007p001G004p001A007p000A0 | 874 | X033A004p001U007p001G004p00 | 1165 |
| 001819 | 04p000U007p000A004p000A007 | | 0G007p000A004p000G007p000G0 | |
| | p000A004p000A007p000G004p0 | | 04p000G007p000C004p000C007p | |
| | 00G007p000C004p000C007p000 | | 0000004p000U007p000U004p000 | |
| | C004p000U007p000C004p000C0 | | U007p000A004p000U007p000U00 | |
| | 07p001A004p001U007 | | 4p000C007p000G004p001C004p0 | |
| | | | 01G004 | |
| DT- | G007p001A004p001A007p000U0 | 875 | X033A004p001A007p001U004p00 | 1166 |
| 001820 | 04p000A007p000A004p000A007 | | 0G007p000G004p000A007p000G0 | |
| | p000A004p000G007p000G004p0 | | 04p000G007p000G004p000C007p | |
| | 00C007p000C004p000C007p000 | | 000C004p000U007p000U004p000 | |
| | U004p000C007p000C004p000A0 | | U007p000U004p000A007p000U00 | |
| | 07p001U004p001U007 | | 4p000U007p000C004p001G004p0 | |
| | | | 01C004 | |

Table 3 below shows the codes in the nucleotide sequences in Table 2 and the following Tables of the disclosure.

TABLE 3

| | |
|---|---|
| p000: phosphate group/linkage | |
| p001: phosphorothioate linker(PS) | |
| SS: sense strand AS: antisense strand | |
| A004: adenosine/2′ OMe | G004: guanosine/2′ OMe |
| A007: adenosine/2′ F | G007: guanosine/2′ F |
| X033A1027: adenosine/5′(E)-VP-2′-OMe | X033G1027: guanosine/5′(E)-VP-2′-OMe |
| X033A004: adenosine/5′-phosphate-2′-OMe | X033G004: guanosine/5′-phosphate-2′-OMe |
| C004: cytidine/2′ OMe | U004: uridine/2′ OMe |
| C007: cytidine/2′ F | U007: uridine/2′ F |
| X033C1027: cytidine/5′(E)-VP-2′-OMe | X033U1027: uridine/5′(E)-VP-2′-OMe |
| X033C004: cytidine/5′-phosphate-2′-OMe | X033U004: uridine/5′-phosphate-2′-OMe |

### Example 4. In Vitro Testing of Unconjugated siRNAs Targeting DMPK

The unconjugated compounds shown in Table 2 were tested for their ability to inhibit the expression of DMPK in human Skeletal Muscle cells (HSkMCs) that express endogenous DMPK. Transfection experiments and DMPK quantitation were performed according to the methods described herein.

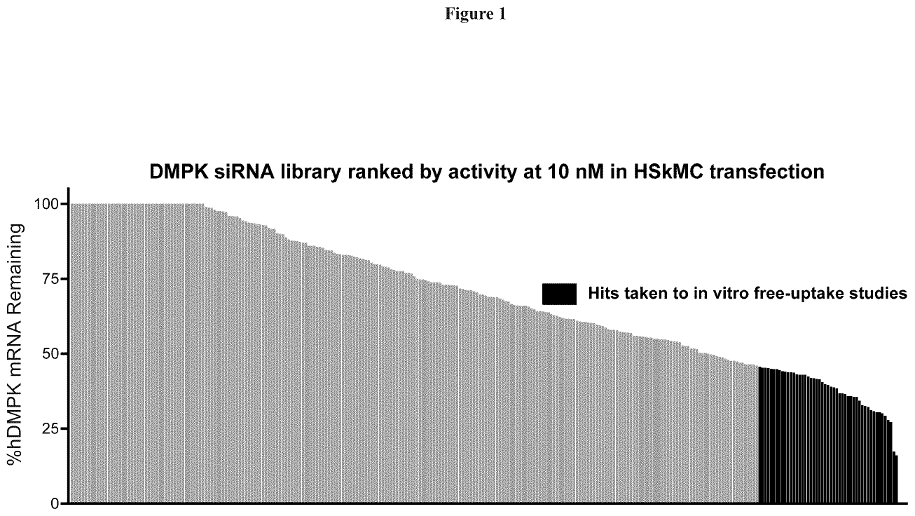

HSkMCs cells were transfected with siRNAs at doses of 0.3 nM, 3 nM, and 30 nM. RNA was isolated 48 hours later, reverse transcribed to cDNA and DMPK expression was quantified by qPCR. The average DMPK expression for each of four replicates was calculated. FIG. 1 shows a representative graph of data from the 10 nM dose. The average DMPK expression for each siRNA was ranked from highest to lowest, and the data were graphed in that order.

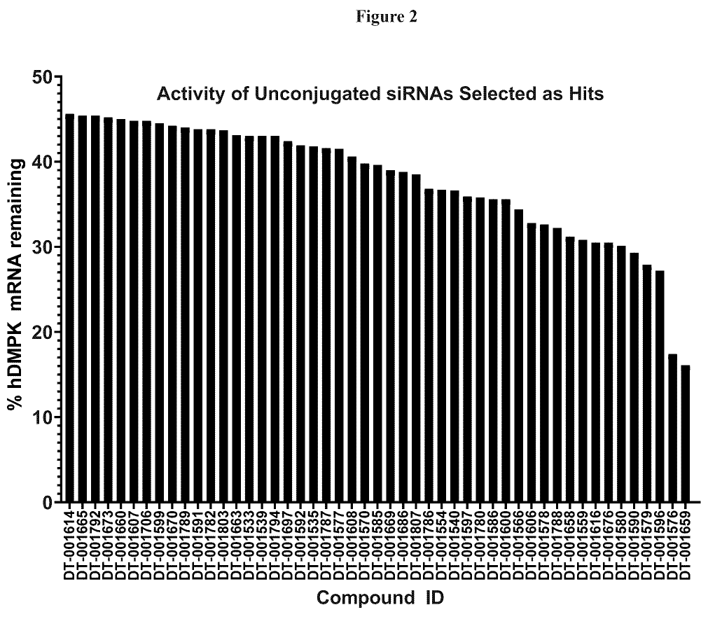

siRNAs resulting in DMPK expression below 46% were considered “hits” and were selected for conjugation to a fatty acid moiety. The expression data for these siRNAs are shown in FIG. 2.

### Example 5. Selection of Fatty Acid Motif

The effectiveness of fatty acid motifs to delivery to muscle tissue in vivo was evaluated using fatty acid motifs of varying structures.

In an initial experiment, two variants of the “DTx-01” scaffold were selected, DTx-01-32 and DTx-01-08 using the DT-001068 siRNA. The DTx-01 scaffold is represented below:

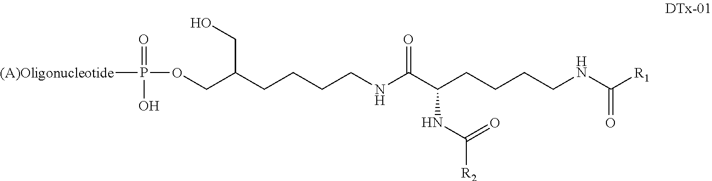

TABLE 4
Fatty acid motifs with DTx-01 scaffold

Motif ID

| | DTx-01-08 | DTx-01-32 |
|---|---|---|
| R¹ | unbranched C₁₅ alkyl | unbranched C₁₃ alkyl |
| R² | unbranched C₁₅ alkyl | unbranched C₁₃ alkyl |

Conjugated compounds were formed as in the structures below, where the phosphate group was attached to the 3′ carbon of the 3′ terminal nucleotide of the sense strand of an siRNA targeted to a DMPK gene mRNA.

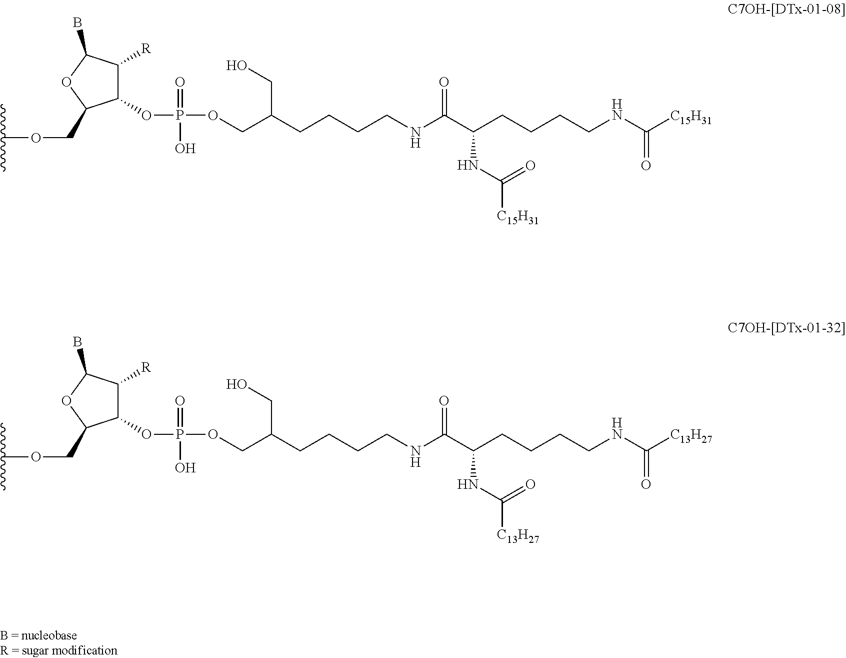

Groups of five mice each were intravenously administered a single dose of PBS or conjugated siRNA at doses indicated in the table below. On Day 7, mice were sacrificed, and RNA was collected from gastrocnemius and transversus abdominus muscle tissues for RNA extraction and quantitation of the muscle-specific DMPK mRNA levels by quantitative RT-PCR as described herein. The average percentage of vehicle expression in each of the collected muscle tissues was calculated for each treatment.

TABLE 5
Comparison of fatty acid motif activities in muscle

| | | DTx-01-08 | | | DTx-01-32 | | |
|---|---|---|---|---|---|---|---|
| | | 5 | 15 | 50 | 5 | 15 | 50 |
| | PBS | mg/kg | mg/kg | mg/kg | mg/kg | mg/kg | mg/kg |
| | Quadricep | | | | | | |
| Mean | 101.9 | 62.2 | 26.8 | 5.6 | 35.3 | 8.3 | 3.1 |
| SEM | 9.6 | 10.1 | 3.0 | 1.7 | 3.7 | 1.1 | 0.3 |

Transversus abdominis

| | | | | | | | |
|---|---|---|---|---|---|---|---|
| Mean | 103.5 | 59.5 | 30.4 | 4.9 | 41.2 | 12.3 | 5.0 |
| SEM | 12.1 | 7.4 | 4.2 | 1.0 | 4.4 | 1.6 | 0.8 |

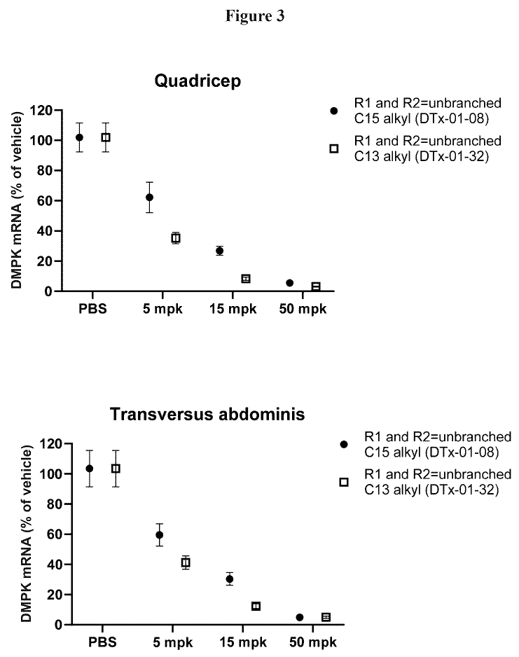

As shown in Table and FIGS. 3A-3B, the DTx-01-32 motif, comprising two unbranched C15 alkyl groups, was more effective than the DTx-01-08 motif in muscle tissue. Accordingly, DTx-01-32 was selected as the fatty acid motif for use in designing DMPK siRNAs.

Additional variants of the DTx-01 scaffold were selected; the fatty acid motifs are shown in the Table below. The motifs were conjugated to the sense strand of the DMPK siRNA and tested in vivo as described above. Of the four motifs tested, only DTx-01-51 performed at least as well as DTx-01-32. The motifs were conjugated to the sense strand of a DMPK siRNA.

TABLE 6
Additional fatty acid motifs with the DTx-01 scaffold

| | DTx-01-30 | DTx-01-51 | DTx-01-54 | DTx-01-55 |
|---|---|---|---|---|
| R¹ | unbranched C₉ | unbranched C₁₃ | unbranched C₁₃ | unbranched C₁₃ |
| | alkyl | alkyl | alkyl | alkyl |
| R² | unbranched C₉ | unbranched C₁₅ | unbranched C₁₅ | unbranched C₁₅ |
| | alkyl | alkyl | alkyl | alkyl |

Further motifs were selected to evaluate variations in the scaffold and unbranched alkyl chain lengths. The scaffold selected was DTx-03, and R¹ and R² are as shown in the Table below. The motifs conjugated to the sense strand of the DMPK siRNA and tested in vivo as described above. Of the four motifs tested, only DTx-03-07, DTx-03-08, and DTx-03-51 performed at least as well as DTx-01-32.

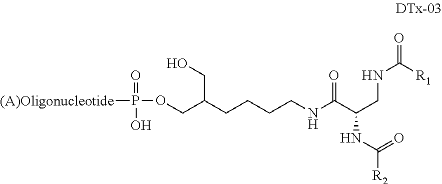

TABLE 7
Fatty acid motifs with the DTx-03 scaffold

| | DTx-03-07 | DTx-03-08 | DTx-03-51 | DTx-03-54 | DTx-03-55 |
|---|---|---|---|---|---|
| R¹ | unbranched C₁₁ alkyl | unbranched C₁₃ alkyl | unbranched C₁₃ alkyl | unbranched C₁₃ alkyl | unbranched C₁₇ alkyl |
| R² | unbranched C₁₁ alkyl | unbranched C₁₃ alkyl | unbranched C₁₅ alkyl | unbranched C₁₇ alkyl | unbranched C₁₃ alkyl |

These data illustrate that variations in fatty acid motif structure affect the in vivo activity of DMPK siRNAs, and that DTx-01-32 is the optimal acid motif for reduction of DMPK mRNA in muscle tissue.

### Example 6. Conjugated siRNAs Targeting DMPK

Using the siRNAs selected as hits in the transfection experiment, a new set of compounds was designed and synthesized to include the fatty acid motif DTx-01-32. DMPK siRNAs were conjugated to the DTx-01-32 conjugate group shown below, where the fatty acid moiety was connected to the 3′ terminal nucleotide of the sense strand via a phosphate group and a C₇OH linker. Conjugated compounds were formed as in the structure shown, where a phosphate group links the 3′ carbon of the 3′ terminal nucleotide and the C₇OH linker, “B” is nucleobase and “R” is the substituent at the 2′ carbon of the nucleoside sugar.

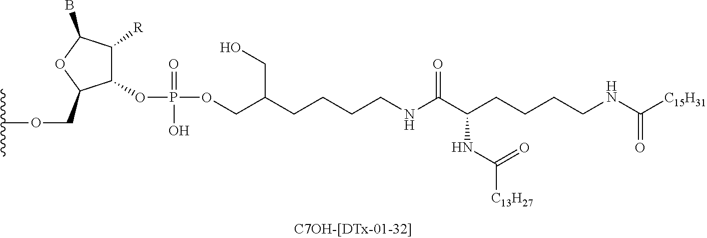

The conjugated compounds are shown in Table 8. Except for the presence of the conjugate group, the siRNA compounds are identical.

TABLE 8
Conjugated siRNAs targeting DMPK

| | | SEQ | | SEQ |
|---|---|---|---|---|
| siRNA | | ID | | ID |
| ID | Sense Strand (modified) | NO. | Antisense Strand (modified) | NO. |
| DT- | C007p001C004p001U007p000C004 | 1167 | X033U004p001A007p001G004p000 | 1217 |
| 001417 | p000G007p000G004p000U007p000 | | A007p000C004p000A007p000A004 | |
| | A004p000U007p000U004p000U007 | | p0000007p000A004p000A007p000 | |
| | p000A004p000U007p000U004p000 | | A004p000U007p000A004p000C007 | |
| | G007p000U004p000C007p001U004 | | p000C004p000G007p000A004p000 | |
| | p001A007p000X2140 | | G007p000G004p001A004p001A004 | |
| DT- | C007p001C004p001A007p000G004 | 1168 | X033U004p001A007p001G004p000 | 1218 |
| 001831 | p000G007p000A004p000U007p000 | | G007p000U004p000A007p000G004 | |
| | G004p000A007p000G004p000A007 | | p0000007p0000004p000C007p000 | |
| | p000A004p000C007p000U004p000 | | U004p000C007p000A004p000U007 | |
| | A007p000C004p000C007p001U004 | | p000C004p000C007p000U004p000 | |
| | p001A007p000X2140 | | G007p000G004p001A004p001A004 | |
| DT- | C007p001U004p001A007p000C004 | 1169 | X033A004p001A007p001U004p000 | 1219 |
| 001832 | p000G007p000U004p000G007p000 | | G007p000U004p000C007p000C004 | |
| | C004p000A007p000C004p000A007 | | p000C007p000U004p000G007p000 | |
| | p000G004p000G007p000G004p000 | | U004p000G007p000C004p000A007 | |
| | A007p000C004p000A007p001U004 | | p000C004p000G007p000U004p000 | |
| | p001U007p000X2140 | | A007p000G004p001C004p001C004 | |
| DT- | U007p001G004p001C007p000A004 | 1170 | X033A004p001G007p001U004p000 | 1220 |
| 001833 | p000C007p000A004p000G007p000 | | U007p000U004p000G007p000A004 | |
| | G004p000G007p000A004p000C007 | | p0000007p000G004p000U007p000 | |
| | p000A004p000U007p000C004p000 | | C004p000C007p000C004p000U007 | |
| | A007p000A004p000A007p001C004 | | p000G004p000U007p000G004p000 | |
| | p001U007p000X2140 | | C007p000A004p001C004p001G004 | |
| DT- | C007p001A004p001C007p000G004 | 1171 | X033A004p001U007p001G004p000 | 1221 |
| 001834 | p000G007p000A004p000U007p000 | | G007p000G004p000A007p000A004 | |
| | C004p000C007p000A004p000C007 | | p000G007p000G004p000U007p000 | |
| | p000C004p000U007p000U004p000 | | G004p000G007p000A004p000U007 | |
| | C007p000C004p000C007p001A004 | | p000C004p000C007p000G004p000 | |
| | p0010007p000X2140 | | U007p000G004p001G004p001C004 | |
| DT- | A007p001U004p001U007p000C004 | 1172 | X033A004p001C007p001A004p000 | 1222 |
| 001835 | p000C007p000U004p000C007p000 | | A007p000U004p000A007p000A004 | |
| | G004p000G007p000U004p000A007 | | p000A007p000U004p000A007p000 | |
| | p0000004p000U007p000U004p000 | | C004p000C007p000G004p000A007 | |
| | A007p000U004p000U007p001G004 | | p000G004p000G007p000A004p000 | |
| | p001U007p000X2140 | | A007p000U004p001G004p001U004 | |
| DT- | C007p001U004p001C007p000G004 | 1173 | X033A004p001C007p001A004p000 | 1223 |
| 001836 | p000G007p000U004p000A007p000 | | G007p000A004p000C007p000A004 | |
| | U004p000U007p000U004p000A007 | | p000A007p000U004p000A007p000 | |
| | p0000004p0000007p000G004p000 | | A004p000A007p000U004p000A007 | |
| | U007p000C004p000U007p001G004 | | p000C004p000C007p000G004p000 | |
| | p0010007p000X2140 | | A007p000G004p001G004p001A004 | |
| DT- | U007p001C004p001G007p000G004 | 1174 | X033A004p001A007p001C004p000 | 1224 |
| 001837 | p0000007p000A004p000U007p000 | | A007p000G004p000A007p000C004 | |
| | U004p000U007p000A004p000U007 | | p000A007p000A004p000U007p000 | |
| | p0000004p000G007p000U004p000 | | A004p000A007p000A004p000U007 | |
| | C007p000U004p000G007p001U004 | | p000A004p000C007p000C004p000 | |
| | p001U007p000X2140 | | G007p000A004p001G004p001G004 | |
| DT- | C007p001G004p001G007p000U004 | 1175 | X033A004p001G007p001A004p000 | 1225 |
| 001838 | p000A007p000U004p000U007p000 | | C007p000A004p000G007p000A004 | |
| | U004p000A007p000U004p000U007 | | p000C007p000A004p000A007p000 | |
| | p000G004p000U007p000C004p000 | | U004p000A007p000A004p000A007 | |
| | U007p000G004p000U007p001C004 | | p0000004p000A007p000C004p000 | |
| | p0010007p000X2140 | | C007p000G004p001A004p001G004 | |
| DT- | C007p001C004p001A007p000U004 | 1176 | X033U004p001U007p001A004p000 | 1226 |
| 001904 | p000C007p000G004p000U007p000 | | A007p000G004p000C007p000C004 | |
| | G004p000G007p000U004p000G007 | | p0000007p000C004p000A007p000 | |
| | p000A004p000G007p000G004p000 | | C004p000C007p000A004p000C007 | |
| | C007p000U004p000U007p001A004 | | p000G004p000A007p000U004p000 | |
| | p001A007p000X2140 | | G007p000G004p001G004p001C004 | |
| DT- | C007p001A004p001U007p000C004 | 1177 | X033A004p001U007p001U004p000 | 1227 |
| 001905 | p000G007p000U004p000G007p000 | | A007p000A004p000G007p000C004 | |
| | G004p000U007p000G004p000A007 | | p000C007p0000004p000C007p000 | |
| | p000G004p000G007p000C004p000 | | A004p000C007p000C004p000A007 | |
| | U007p000U004p000A007p001A004 | | p000C004p000G007p000A004p000 | |
| | p0010007p000X2140 | | U007p000G004p001G004p001G004 | |
| DT- | G007p001G004p001A007p000C004 | 1178 | X033A004p001A007p001G004p000 | 1228 |
| 001906 | p000G007p000A004p000C007p000 | | A007p000A004p000U007p000C004 | |
| | U004p000U007p000C004p000G007 | | p0000007p000C004p000G007p000 | |
| | p000A004p000G007p000A004p000 | | A004p000A007p000G004p000U007 | |
| | U007p000U004p000C007p001U004 | | p000C004p000G007p000U004p000 | |
| | p0010007p000X2140 | | C007p000C004p001C004p001U004 | |
| DT- | A007p001G004p001C007p000A004 | 1179 | X033U004p001A007p001C004p000 | 1229 |
| 001907 | p000G007p000A004p000C007p000 | | A007p000C004p000C007p000U004 | |
| | G004p000G007p000G004p000C007 | | p000G007p000G004p000C007p000 | |
| | p000C004p000A007p000G004p000 | | C004p000C007p000G004p000U007 | |
| | G007p000U004p000G007p001U004 | | p000004p000U007p000G004p000 | |
| | p001A007p000X2140 | | C007p000U004p001U004p001C004 | |
| DT- | C007p001A004p001U007p000G004 | 1180 | X033A004p001U007p001U004p000 | 1230 |
| 001908 | p000A007p000A004p000G007p000 | | G007p000U004p000U007p000C004 | |
| | A004p000U007p000C004p000A007 | | p000A007p000U004p000G007p000 | |
| | p0000004p000G007p000A004p000 | | A004p000U007p000C004p000U007 | |
| | A007p000C004p000A007p001A004 | | p0000004p000C007p000A004p000 | |
| | p001U007p000X2140 | | U007p000G004p001G004p001C004 | |
| DT- | U007p001G004p001A007p000A004 | 1181 | X033A004p001A007p001C004p000 | 1231 |
| 001909 | p000G007p000A004p000U007p000 | | U007p000U004p000G007p000U004 | |
| | C004p000A007p000U004p000G007 | | p0000007p000C004p000A007p000 | |
| | p000A004p000A007p000C004p000 | | U004p000G007p000A004p000U007 | |
| | A007p000A004p000G007p001U004 | | p000C004p0000007p000U004p000 | |
| | p001U007p000X2140 | | C007p000A004p001U004p001G004 | |
| DT- | G007p001A004p001G007p000G004 | 1182 | X033A004p001A007p001C004p000 | 1232 |
| 001910 | p0000007p000G004p000U007p000 | | G007p000G004p000A007p000A004 | |
| | C004p000G007p000U004p000G007 | | p000G007p000C004p000A007p000 | |
| | p000C004p000U007p000U004p000 | | C004p000G007p000A004p000C007 | |
| | C007p000C004p000G007p001U004 | | p000A004p000C007p000C004p000 | |
| | p0010007p000X2140 | | U007p000C004p001G004p001C004 | |
| DT- | G007p001A004p001G007p000G004 | 1183 | X033A004p001U007p001U004p000 | 1233 |
| 001911 | p000G007p000A004p000C007p000 | | C007p000A004p000C007p000C004 | |
| | G004p000U007p000G004p000U007 | | p000A007p000A004p000C007p000 | |
| | p0000004p000G007p000G004p000 | | A004p000C007p000G004p000U007 | |
| | U007p000G004p000A007p001A004 | | p0000004p000C007p000C004p000 | |
| | p001U007p000X2140 | | U007p000C004p001U004p001C004 | |
| DT- | G007p001G004p001U007p000C004 | 1184 | X033A004p001A007p001C004p000 | 1234 |
| 001912 | p000A007p000U004p000G007p000 | | G007p000U004p000A007p000A004 | |
| | G004p000A007p000G004p000U007 | | p0000007p000A004p000C007p000 | |
| | p000A004p000U007p000U004p000 | | U004p000C007p000C004p000A007 | |
| | A007p000C004p000G007p001U004 | | p0000004p000G007p000A004p000 | |
| | p001U007p000X2140 | | C007p000C004p001A004p001G004 | |
| DT- | U007p001U004p001C007p000G004 | 1185 | X033A004p001G007p001A004p000 | 1235 |
| 002008 | p000C007p000C004p000U007p000 | | A007p000C004p000A007p000U004 | |
| | A004p000U007p000G004p000A007 | | p0000007p0000004p000C007p000 | |
| | p000A004p000A007p000U004p000 | | A004p000U007p000A004p000G007 | |
| | G007p000U004p000U007p001C004 | | p000G004p000C007p000G004p000 | |
| | p001U007p000X2140 | | A007p000A004p001U004p001A004 | |
| DT- | U007p001C004p001G007p000C004 | 1186 | X033U004p001A007p001G004p000 | 1236 |
| 002009 | p000C007p0000004p000A007p000 | | A007p000A004p000C007p000A004 | |
| | U004p000G007p000A004p000A007 | | p0000007p0000004p000U007p000 | |
| | p000A004p000U007p000G004p000 | | C004p000A007p000U004p000A007 | |
| | U007p000U004p000C007p001U004 | | p000G004p000G007p000C004p000 | |
| | p001A007p000X2140 | | G007p000A004p001A004p001U004 | |
| DT- | G007p001G004p001G007p000C004 | 1187 | X033A004p001G007p001U004p000 | 1237 |
| 002010 | p000A007p000G004p000A007p000 | | A007p000G004p000A007p000A004 | |
| | C004p000G007p000C004p000C007 | | p000G007p000G004p000G007p000 | |
| | p000C004p000U007p000U004p000 | | C004p000G007p000U004p000C007 | |
| | C007p000U004p000A007p001C004 | | p0000004p000G007p000C004p000 | |
| | p0010007p000X2140 | | C007p000C004p001A004p001U004 | |
| DT- | C007p001G004p001C007p000C004 | 1188 | X033A004p001A007p001A004p000 | 1238 |
| 002011 | p000C007p0000004p000U007p000 | | U007p000C004p000C007p000G004 | |
| | C004p0000007p000A004p000C007 | | p000C007p000G004p000U007p000 | |
| | p000G004p000C007p000G004p000 | | A004p000G007p000A004p000A007 | |
| | G007p000A004p000U007p001U004 | | p000G004p000G007p000G004p000 | |
| | p001U007p000X2140 | | C007p000G004p001U004p001C004 | |
| DT- | C007p001C004p001U007p000U004 | 1189 | X033A004p001U007p001G004p000 | 1239 |
| 002012 | p000C007p000U004p000A007p000 | | G007p000A004p000A007p000U004 | |
| | C004p000G007p000C004p000G007 | | p000C007p000C004p000G007p000 | |
| | p000G004p000A007p000U004p000 | | C004p000G007p000U004p000A007 | |
| | U007p000C004p000C007p001A004 | | p000G004p000A007p000A004p000 | |
| | p001U007p000X2140 | | G007p000G004p001G004p001C004 | |
| DT- | C007p001A004p001A007p000G004 | 1190 | X033C004p001U007p001U004p000 | 1240 |
| 002013 | p000A007p000U004p000C007p000 | | G007p000U004p000A007p000G004 | |
| | G004p000U007p000C004p000C007 | | p0000007p000G004p000G007p000 | |
| | p000A004p000C007p000U004p000 | | A004p000C007p000G004p000A007 | |
| | A007p000C004p000A007p001A004 | | p0000004p000C007p0000004p000 | |
| | p001G007p000X2140 | | U007p000G004p001C004p001C004 | |
| DT- | C007p001U004p001A007p000C004 | 1191 | X033A004p001G007p001A004p000 | 1241 |
| 002014 | p000A007p000A004p000G007p000 | | G007p000A004p000G007p000G004 | |
| | G004p000A007p000G004p000C007 | | p0000007p000G004p000C007p000 | |
| | p000A004p000C007p000C004p000 | | U004p000C007p000C004p000U007 | |
| | U007p000C004p000U007p001C004 | | p0000004p000G007p000U004p000 | |
| | p001U007p000X2140 | | A007p000G004p001U004p001G004 | |
| DT- | C007p001A004p001A007p000G004 | 1192 | X033A004p001A007p001G004p000 | 1242 |
| 002015 | p000G007p000A004p000G007p000 | | A007p000G004p000A007p000G004 | |
| | C004p000A007p000C004p000C007 | | p000A007p000G004p000G007p000 | |
| | p0000004p000C007p000U004p000 | | U004p000G007p000C004p000U007 | |
| | C007p000U004p000C007p001U004 | | p000C004p000C007p000U004p000 | |
| | p0010007p000X2140 | | U007p000G004p001U004p001A004 | |
| DT- | C007p001U004p001U007p000C004 | 1193 | X033A004p001U007p001C004p000 | 1243 |
| 002023 | p0000007p000U004p000C007p000 | | G007p000A004p000G007p000G004 | |
| | U004p000U007p000U004p000G007 | | p000C007p000C004p000A007p000 | |
| | p000G004p000C007p000C004p000 | | A004p000A007p000G004p000A007 | |
| | U007p000C004p000G007p001A004 | | p000A004p000G007p000A004p000 | |
| | p0010007p000X2140 | | A007p000G004p001G004p001G004 | |
| DT- | U007p001C004p001G007p000A004 | 1194 | X033A004p001U007p001G004p000 | 1244 |
| 002024 | p000A007p000G004p000G007p000 | | U007p000C004p000G007p000G004 | |
| | U004p000G007p000C004p000C007 | | p0000007p000G004p000G007p000 | |
| | p000A004p000C007p000C004p000 | | C004p000A007p000C004p000C007 | |
| | G007p000A004p000C007p001A004 | | p0000004p000U007p000C004p000 | |
| | p001U007p000X2140 | | G007p000A004p001A004p001A004 | |
| DT- | A007p001C004p001A007p000C004 | 1195 | X033A004p001A007p001G004p000 | 1245 |
| 002025 | p000A007p000U004p000G007p000 | | U007p000C004p000G007p000A004 | |
| | C004p000A007p000A004p000C007 | | p000A007p000G004p000U007p000 | |
| | p0000004p000U007p000C004p000 | | U004p000G007p000C004p000A007 | |
| | G007p000A004p000C007p001U004 | | p0000004p000G007p000U004p000 | |
| | p0010007p000X2140 | | G007p000U004p001C004p001G004 | |
| DT- | C007p001A004p001A007p000C004 | 1196 | X033A004p001U007p001C004p000 | 1246 |
| 002026 | p0000007p000U004p000C007p000 | | C007p000A004p000C007p000C004 | |
| | G004p000A007p000C004p000U007 | | p000A007p000A004p000G007p000 | |
| | p0000004p000G007p000G004p000 | | U004p000C007p000G004p000A007 | |
| | U007p000G004p000G007p001A004 | | p000A004p000G007p000U004p000 | |
| | p0010007p000X2140 | | U007p000G004p001C004p001A004 | |
| DT- | G007p001C004p001A007p000G004 | 1197 | X033A004p001A007p001U004p000 | 1247 |
| 002027 | p0000007p000U004p000G007p000 | | C007p000C004p000G007p000C004 | |
| | C004p000A007p000G004p000G007 | | p0000007p000C004p000C007p000 | |
| | p000A004p000G007p000C004p000 | | U004p000G007p000C004p000A007 | |
| | G007p000G004p000A007p001U004 | | p000A004p000C007p000U004p000 | |
| | p0010007p000X2140 | | G007p000C004p001C004p001G004 | |
| DT- | U007p001G004p001C007p000A004 | 1198 | X033A004p001A007p001C004p000 | 1248 |
| 002028 | p000G007p000G004p000A007p000 | | U007p000C004p000C007p000A004 | |
| | G004p000C007p000G004p000G007 | | p0000007p000C004p000C007p000 | |
| | p000A004p000U007p000G004p000 | | G004p000C007p000U004p000C007 | |
| | G007p000A004p000G007p001U004 | | p000C004p000U007p000G004p000 | |
| | p0010007p000X2140 | | C007p000A004p001A004p001C004 | |
| DT- | G007p001C004p001C007p000U004 | 1199 | X033U004p001A007p001A004p000 | 1249 |
| 002029 | p000A007p000U004p000C007p000 | | A007p000G004p000C007p000G004 | |
| | G004p000G007p000A004p000G007 | | p000C007p000C004p000U007p000 | |
| | p000G004p000C007p000G004p000 | | C004p000C007p000G004p000A007 | |
| | C007p000U004p000U007p001U004 | | p0000004p000A007p000G004p000 | |
| | p001A007p000X2140 | | G007p000C004p001C004p001A004 | |
| DT- | G007p001A004p001A007p000C004 | 1200 | X033A004p001A007p001A004p000 | 1250 |
| 002030 | p000C007p000C004p000U007p000 | | G007p000A004p000C007p000A004 | |
| | A004p000G007p000A004p000A007 | | p000G007p000U004p000U007p000 | |
| | p000C004p000U007p000G004p000 | | C004p000U007p000A004p000G007 | |
| | U007p000C004p000U007p001U004 | | p000G004p000G007p000U004p000 | |
| | p0010007p000X2140 | | U007p000C004p001A004p001G004 | |
| DT- | A007p001A004p001C007p000C004 | 1201 | X033A004p001G007p001A004p000 | 1251 |
| 002031 | p000C007p000U004p000A007p000 | | A007p000G004p000A007p000C004 | |
| | G004p000A007p000A004p000C007 | | p000A007p000G004p000U007p000 | |
| | p0000004p000G007p000U004p000 | | U004p000C007p000U004p000A007 | |
| | C007p000U004p000U007p001C004 | | p000G004p000G007p000G004p000 | |
| | p0010007p000X2140 | | U007p000U004p001C004p001A004 | |
| DT- | U007p001U004p001A007p000C004 | 1202 | X033A004p001A007p001G004p000 | 1252 |
| 002032 | p0000007p000C004p000G007p000 | | C007p000A004p000A007p000A004 | |
| | G004p000G007p000A004p000A007 | | p0000007p0000004p0000007p000 | |
| | p000A004p000U007p000U004p000 | | C004p000C007p000C004p000G007 | |
| | U007p000G004p000C007p001U004 | | p000A004p000G007p0000004p000 | |
| | p001U007p000X2140 | | A007p000A004p001G004p001C004 | |
| DT- | U007p001C004p001G007p000G004 | 1203 | X033U004p001C007p001A004p000 | 1253 |
| 002033 | p000G007p000A004p000A007p000 | | A007p000A004p000A007p000G004 | |
| | A004p000U007p0000004p000U007 | | p000C007p000A004p000A007p000 | |
| | p000G004p000C007p000U004p000 | | A004p000U007p000U004p000U007 | |
| | U007p000U004p000U007p001G004 | | p000C004p000C007p000C004p000 | |
| | p0010007p000X2140 | | G007p000A004p001G004p001U004 | |
| DT- | G007p001A004p001A007p000A004 | 1204 | X033U004p001U007p001U004p000 | 1254 |
| 002034 | p0000007p0000004p000U007p000 | | G007p000G004p000C007p000A004 | |
| | G004p000C007p000U004p000U007 | | p000A007p000A004p000A007p000 | |
| | p0000004p000U007p000G004p000 | | G004p000C007p000A004p000A007 | |
| | C007p000C004p000A007p001A004 | | p000A004p000U007p000U004p000 | |
| | p001A007p000X2140 | | U007p000C004p001C004p001C004 | |
| DT- | C007p001U004p001U007pC004pG0 | 1205 | X033A004p001A007p001U004p000 | 1255 |
| 002035 | 07pG004pC007pG004pG007pU004p | | A007p000U004p000C007p000C004 | |
| | U007pU004pG007pG004pA007pU00 | | p000A007p000A004p000A007p000 | |
| | 4pA007p001U004p001U007px2140 | | C004p000C007p000G004p000C007 | |
| | | | p000C004p000G007p000A004p000 | |
| | | | A007p000G004p001C004p001G004 | |
| DT- | U007p001C004p001G007p000G004 | 1206 | X033U004p001A007p001A004p000 | 1256 |
| 002085 | p000C007p000G004p000G007p000 | | A007p000U004p000A007p000U004 | |
| | U004p000U007p000U004p000G007 | | p000C007p000C004p000A007p000 | |
| | p000G004p000A007p000U004p000 | | A004p000A007p000C004p000C007 | |
| | A007p0000004p0000007p001U004 | | p000G004p000C007p000C004p000 | |
| | p001A007p000X2140 | | G007p000A004p001A004p001G004 | |
| DT- | C007p001G004p001G007p000U004 | 1207 | X033U004p001C007p001A004p000 | 1257 |
| 002086 | p0000007p000U004p000G007p000 | | A007p000U004p000A007p000A004 | |
| | G004p000A007p000U004p000A007 | | p000A007p000U004p000A007p000 | |
| | p0000004p0000007p000U004p000 | | U004p000C007p000C004p000A007 | |
| | A007p000U004p000U007p001G004 | | p000A004p000A007p000C004p000 | |
| | p001A007p000X2140 | | C007p000G004p001C004p001C004 | |
| DT- | G007p001G004p001U007p000U004 | 1208 | X033A004p001U007p001C004p000 | 1258 |
| 002087 | p0000007p000G004p000G007p000 | | A007p000A004p000U007p000A004 | |
| | A004p000U007p000A004p000U007 | | p000A007p000A004p000U007p000 | |
| | p0000004p0000007p000A004p000 | | A004p000U007p000C004p000C007 | |
| | U007p000U004p000G007p001A004 | | p000A004p000A007p000A004p000 | |
| | p001U007p000X2140 | | C007p000C004p001G004p001C004 | |
| DT- | G007p001U004p001U007p000U004 | 1209 | X033A004p001G007p001U004p000 | 1259 |
| 002088 | p000G007p000G004p000A007p000 | | C007p000A004p000A007p000U004 | |
| | U004p000A007p000U004p000U007 | | p000A007p000A004p000A007p000 | |
| | p0000004p000A007p000U004p000 | | U004p000A007p000U004p000C007 | |
| | U007p000G004p000A007p001C004 | | p000C004p000A007p000A004p000 | |
| | p001U007p000X2140 | | A007p000C004p001C004p001G004 | |
| DT- | G007p001G004p001A007p000U004 | 1210 | X033A004p001C007p001G004p000 | 1260 |
| 002089 | p000A007p000U004p000U007p000 | | A007p000G004p000G007p000U004 | |
| | U004p000A007p000U004p000U007 | | p000C007p000A004p000A007p000 | |
| | p000G004p000A007p000C004p000 | | U004p000A007p000A004p000A007 | |
| | C007p000U004p000C007p001G004 | | p0000004p000A007p000U004p000 | |
| | p0010007p000X2140 | | C007p000C004p001A004p001A004 | |
| DT- | G007p001A004p001U007p000A004 | 1211 | X033U004p001A007p001C004p000 | 1261 |
| 002090 | p0000007p0000004p000U007p000 | | G007p000A004p000G007p000G004 | |
| | A004p000U007p000U004p000G007 | | p0000007p000C004p000A007p000 | |
| | p000A004p000C007p000C004p000 | | A004p000U007p000A004p000A007 | |
| | U007p000C004p000G007p001U004 | | p000A004p000U007p000A004p000 | |
| | p001A007p000X2140 | | U007p000C004p001C004p001A004 | |
| DT- | A007p001U004p001A007p000U004 | 1212 | X033A004p001G007p001A004p000 | 1262 |
| 002091 | p0000007p000U004p000A007p000 | | C007p000G004p000A007p000G004 | |
| | U004p000U007p000G004p000A007 | | p000G007p000U004p000C007p000 | |
| | p000C004p000C007p000U004p000 | | A004p000A007p000U004p000A007 | |
| | C007p000G004p000U007p001C004 | | p000A004p000A007p000U004p000 | |
| | p001U007p000X2140 | | A007p000U004p001C004p001C004 | |
| DT- | C007p001C004p001C007p000A004 | 1213 | X033A004p001U007p001C004p000 | 1263 |
| 002092 | p000A007p000U004p000C007p000 | | C007p000A004p000A007p000A004 | |
| | C004p000A007p000C004p000G007 | | p000A007p000C004p000G007p000 | |
| | p0000004p0000007p000U004p000 | | U004p000G007p000G004p000A007 | |
| | U007p000G004p000G007p001A004 | | p0000004p0000007p000G004p000 | |
| | p0010007p000X2140 | | G007p000G004p001U004p001G004 | |
| DT- | A007p001U004p001C007p000C004 | 1214 | X033A004p001U007p001G004p000 | 1264 |
| 002093 | p000A007p000C004p000G007p000 | | C007p000A004p000U007p000C004 | |
| | U004p0000007p000U004p000007 | | p000C007p000A004p000A007p000 | |
| | p000G004p000G007p000A004p000 | | A004p000A007p000C004p000G007 | |
| | U007p000G004p000C007p001A004 | | p0000004p000G007p000G004p000 | |
| | p0010007p000X2140 | | A007p000U004p001U004p001G004 | |
| DT- | C007p001C004p001A007p000C004 | 1215 | X033A004p001A007p001G004p000 | 1265 |
| 002094 | p000G007p000U004p000U007p000 | | U007p000G004p000C007p000A004 | |
| | U004p000U007p000G004p000G007 | | p0000007p000C004p000C007p000 | |
| | p000A004p000U007p000G004p000 | | A004p000A007p000A004p000A007 | |
| | C007p000A004p000C007p001U004 | | p000C004p000G007p000U004p000 | |
| | p0010007p000X2140 | | G007p000G004p001A004p001U004 | |
| DT- | C007p001G004p001U007p000U004 | 1216 | X033U004p001C007p001U004p000 | 1266 |
| 002095 | p0000007p0000004p000G007p000 | | C007p000A004p000G007p000U004 | |
| | G004p000A007p000U004p000G007 | | p000G007p000C004p000A007p000 | |
| | p000C004p000A007p000C004p000 | | U004p000C007p000C004p000A007 | |
| | U007p000G004p000A007p001G004 | | p000A004p000A007p000A004p000 | |
| | p001A007p000X2140 | | C007p000G004p001U004p001G004 | |

X2140:

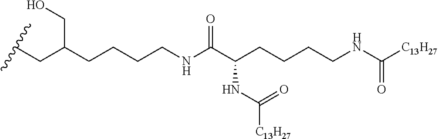

In Table 8, modified nucleotides are indicated with the codes described in Table 3.

### Example 7. Free Uptake Experiments to Select siRNAs for In Vivo Testing

Conjugated siRNAs were tested for their ability to inhibit the expression of DMPK in HSkMCs. These studies were performed under free uptake conditions as described herein (i.e., no transfection reagent was used).

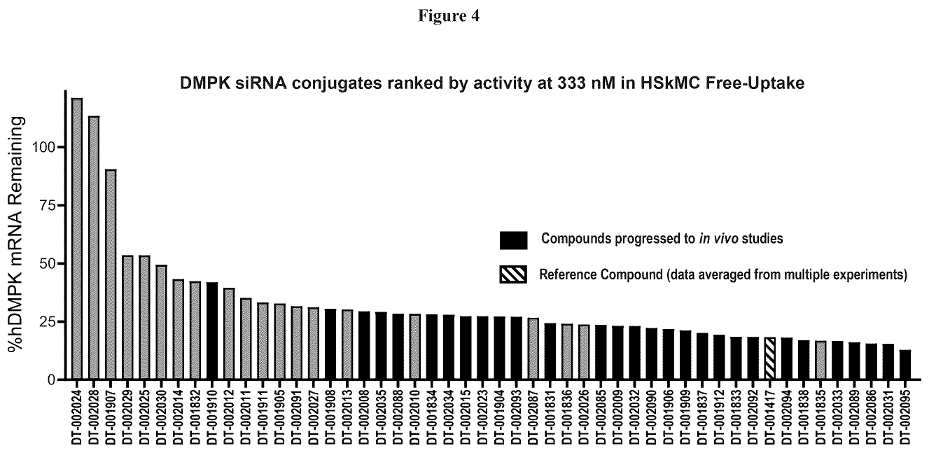

Free uptake experiments were performed as described herein. HSkMCs were treated with 333 nM of conjugated siRNA. RNA was isolated 48 hours later, reverse transcribed to cDNA and DMPK expression was quantified by qPCR. The average DMPK expression for each of four replicates was calculated. The average DMPK expression for each conjugated siRNA was ranked from highest to lowest, and the data were graphed in that order. The resulting graph is shown in FIG. 4.

As shown in FIG. 4, the conjugated siRNAs exhibited varying inhibitory activities. Conjugated siRNAs with inhibitory activity comparable to that of DT-001417 (identified as active in preliminary experiments) were selected for progression to in vivo target engagement studies. For these in vivo experiments, a new set of compounds was designed and synthesized to include a 5′-VP group at the 5′-terminus of the antisense strand. Table 8 shows the sequences and chemical modifications of the sense and antisense strands of each conjugated siRNA. Each sense and antisense strand is assigned a unique “Strand ID”; for each siRNA, the sense strand comprises the C70H-DTx-01-32 conjugate (X2140) and the antisense strand comprises the 5′-VP modification. The corresponding parent compound IDs (i.e., conjugated siRNAs without 5′-VP) are shown in Table 8.

In Table 9, modified nucleotides are indicated with the codes described in Table 3.

TABLE 9
siRNA Compounds for in vivo studies

| | | | SEQ |
|---|---|---|---|
| siRNA | | | ID |
| ID | strand | Modified sequence | No. |
| DT- | SS | C007p001C004p001U007p000C004p000G007p000G004p000U007p0 | 1267 |
| 001068 | | 00A004p000U007p000U004p000U007p000A004p000U007p000U004 | |
| | | p000G007p000U004p000C007p001U004p001A007p000X2140 | |
| | AS | X033U1027p001A007p001G004p000A007p000C004p000A007p000A | 1268 |
| | | 004p0000007p000A004p000A007p000A004p000U007p000A004p00 | |
| | | 0C007p000C004p000G007p000A004p000G007p000G004p001A004p | |
| | | 001A004 | |
| DT- | SS | U007p001G004p001C007p000A004p000C007p000A004p000G007p0 | 1269 |
| 002039 | | 00G004p000G007p000A004p000C007p000A004p000U007p000C004 | |
| | | p000A007p000A004p000A007p001C004p001U007p000X2140 | |
| | AS | X033A1027p001G007p001U004p000U007p000U004p000G007p000A | 1270 |
| | | 004p000U007p000G004p000U007p000C004p000C007p000C004p00 | |
| | | 0U007p000G004p000U007p000G004p000C007p000A004p001C004p | |
| | | 001G004 | |
| DT- | SS | C007p001A004p001C007p000G004p000G007p000A004p000U007p0 | 1271 |
| 002040 | | 00C004p000C007p000A004p000C007p000C004p000U007p000U004 | |
| | | p000C007p000C004p000C007p001A004p001U007p000X2140 | |
| | AS | X033A1027p001U007p001G004p000G007p000G004p000A007p000A | 1272 |
| | | 004p000G007p000G004p000U007p000G004p000G007p000A004p00 | |
| | | OU007p000C004p000C007p000G004p000U007p000G004p001G004p | |
| | | 001C004 | |
| DT- | SS | U007p001C004p001G007p000G004p000U007p000A004p000U007p0 | 1273 |
| 002041 | | 000004p000U007p000A004p000U007p000U004p000G007p000U004 | |
| | | p000C007p0000004p000G007p001U004p001U007p000X2140 | |
| | AS | X033A1027p001A007p001C004p000A007p000G004p000A007p000C | 1274 |
| | | 004p000A007p000A004p000U007p000A004p000A007p000A004p00 | |
| | | 0U007p000A004p000C007p000C004p000G007p000A004p001G004p | |
| | | 001G004 | |
| DT- | SS | C007p001C004p001A007p000U004p000C007p000G004p000U007p0 | 1275 |
| 002042 | | 00G004p000G007p000U004p000G007p000A004p000G007p000G004 | |
| | | p000C007p000U004p000U007p001A004p001A007p000X2140 | |
| | AS | X033U1027p001U007p001A004p000A007p000G004p000C007p000C | 1276 |
| | | 004p000U007p000C004p000A007p000C004p000C007p000A004p00 | |
| | | 0C007p000G004p000A007p000U004p000G007p000G004p001G004p | |
| | | 001C004 | |
| DT- | SS | G007p001G004p001A007p000C004p000G007p000A004p000C007p0 | 1277 |
| 002043 | | 00004p000U007p000C004p000G007p000A004p000G007p000A004 | |
| | | p0000007p0000004p000C007p001U004p001U007p000X2140 | |
| | AS | X033A1027p001A007p001G004p000A007p000A004p000U007p000C | 1278 |
| | | 004p000U007p000C004p000G007p000A004p000A007p000G004p00 | |
| | | 0U007p000C004p000G007p000U004p000C007p000C004p001C004p | |
| | | 001U004 | |
| DT- | SS | U007p001G004p001A007p000A004p000G007p000A004p000U007p0 | 1279 |
| 002044 | | 00C004p000A007p000U004p000G007p000A004p000A007p000C004 | |
| | | p000A007p000A004p000G007p001U004p001U007p000X2140 | |
| | AS | X033A1027p001A007p001C004p000U007p000U004p000G007p000U | 1280 |
| | | 004p000U007p000C004p000A007p000U004p000G007p000A004p00 | |
| | | OU007p000C004p000U007p000U004p000C007p000A004p001U004p | |
| | | 001G004 | |
| DT- | SS | G007p001A004p001G007p000G004p000U007p000G004p000U007p0 | 1281 |
| 002045 | | 00C004p000G007p000U004p000G007p000C004p000U007p000U004 | |
| | | p000C007p000C004p000G007p001U004p001U007p000X2140 | |
| | AS | X033A1027p001A007p001C004p000G007p000G004p000A007p000A | 1282 |
| | | 004p000G007p000C004p000A007p000C004p000G007p000A004p00 | |
| | | 0C007p000A004p000C007p000C004p000U007p000C004p001G004p | |
| | | 001C004 | |
| DT- | SS | G007p001G004p001U007p000C004p000A007p000U004p000G007p0 | 1283 |
| 002046 | | 00G004p000A007p000G004p000U007p000A004p000U007p000U004 | |
| | | p000A007p000C004p000G007p001U004p001U007p000X2140 | |
| | AS | X033A1027p001A007p001C004p000G007p000U004p000A007p000A | 1284 |
| | | 004p000U007p000A004p000C007p000U004p000C007p000C004p00 | |
| | | 0A007p000U004p000G007p000A004p000C007p000C004p001A004p | |
| | | 001G004 | |
| DT- | SS | C007p001C004p001A007p000G004p000G007p000A004p000U007p0 | 1285 |
| 002097 | | 00G004p000A007p000G004p000A007p000A004p000C007p000U004 | |
| | | p000A007p000C004p000C007p001U004p001A007p000X2140 | |
| | AS | X033U1027p001A007p001G004p000G007p000U004p000A007p000G | 1286 |
| | | 004p000U007p000U004p000C007p000U004p000C007p000A004p00 | |
| | | OU007p000C004p000C007p000U004p000G007p000G004p001A004p | |
| | | 001A004 | |
| DT- | SS | C007p001G004p001G007p000U004p000A007p000U004p000U007p0 | 1287 |
| 002098 | | 000004p000A007p000U004p000U007p000G004p000U007p000C004 | |
| | | p0000007p000G004p000U007p001C004p001U007p000X2140 | |
| | AS | X033A1027p001G007p001A004p000C007p000A004p000G007p000A | 1288 |
| | | 004p000C007p000A004p000A007p000U004p000A007p000A004p00 | |
| | | 0A007p000U004p000A007p000C004p000C007p000G004p001A004p | |
| | | 001G004 | |
| DT- | SS | C007p001A004p001U007p000G004p000A007p000A004p000G007p0 | 1289 |
| 002099 | | 00A004p000U007p000C004p000A007p000U004p000G007p000A004 | |
| | | p000A007p000C004p000A007p001A004p001U007p000X2140 | |
| | AS | X033A1027p001U007p001U004p000G007p000U004p000U007p000C | 1290 |
| | | 004p000A007p000U004p000G007p000A004p000U007p000C004p00 | |
| | | 0U007p000U004p000C007p000A004p000U007p000G004p001G004p | |
| | | 001C004 | |
| DT- | SS | U007p001U004p001C007p000G004p000C007p000C004p000U007p0 | 1291 |
| 002100 | | 00A004p000U007p000G004p000A007p000A004p000A007p000U004 | |
| | | p000G007p000U004p000U007p001C004p001U007p000X2140 | |
| | AS | X033A1027p001G007p001A004p000A007p000C004p000A007p000U | 1292 |
| | | 004p000U007p000U004p000C007p000A004p000U007p000A004p00 | |
| | | 0G007p000G004p000C007p000G004p000A007p000A004p001U004p | |
| | | 001A004 | |
| DT- | SS | U007p001C004p001G007p000C004p000C007p000U004p000A007p0 | 1293 |
| 002101 | | 000004p000G007p000A004p000A007p000A004p000U007p000G004 | |
| | | p0000007p0000004p000C007p001U004p001A007p000X2140 | |
| | AS | X033U1027p001A007p001G004p000A007p000A004p000C007p000A | 1294 |
| | | 004p0000007p000U004p000U007p000C004p000A007p000U004p00 | |
| | | 0A007p000G004p000G007p000C004p000G007p000A004p001A004p | |
| | | 001U004 | |
| DT- | SS | C007p001A004p001A007p000G004p000G007p000A004p000G007p0 | 1295 |
| 002102 | | 00C004p000A007p000C004p000C007p000U004p000C007p000U004 | |
| | | p000C007p000U004p000C007p001U004p001U007p000X2140 | |
| | AS | X033A1027p001A007p001G004p000A007p000G004p000A007p000G | 1296 |
| | | 004p000A007p000G004p000G007p000U004p000G007p000C004p00 | |
| | | 0U007p000C004p000C007p000U004p000U007p000G004p001U004p | |
| | | 001A004 | |
| DT- | SS | C007p001U004p001U007p000C004p000U007p000U004p000C007p0 | 1297 |
| 002164 | | 000004p000U007p000U004p000G007p000G004p000C007p000C004 | |
| | | p0000007p000C004p000G007p001A004p001U007p000X2140 | |
| | AS | X033A1027p001U007p001C004p000G007p000A004p000G007p000G | 1298 |
| | | 004p000C007p000C004p000A007p000A004p000A007p000G004p00 | |
| | | 0A007p000A004p000G007p000A004p000A007p000G004p001G004p | |
| | | 001G004 | |
| DT- | SS | A007p001A004p001C007p000C004p000C007p000U004p000A007p0 | 1299 |
| 002165 | | 00G004p000A007p000A004p000C007p000U004p000G007p000U004 | |
| | | p000C007p0000004p000U007p001C004p001U007p000X2140 | |
| | AS | X033A1027p001G007p001A004p000A007p000G004p000A007p000C | 1300 |
| | | 004p000A007p000G004p000U007p000U004p000C007p000U004p00 | |
| | | 0A007p000G004p000G007p000G004p000U007p000U004p001C004p | |
| | | 001A004 | |
| DT- | SS | U007p001U004p001A007p000C004p000U007p000C004p000G007p0 | 1301 |
| 002166 | | 00G004p000G007p000A004p000A007p000A004p000U007p000U004 | |
| | | p0000007p000G004p000C007p001U004p001U007p000X2140 | |
| | AS | X033A1027p001A007p001G004p000C007p000A004p000A007p000A | 1302 |
| | | 004p000U007p000U004p000U007p000C004p000C007p000C004p00 | |
| | | 0G007p000A004p000G007p000U004p000A007p000A004p001G004p | |
| | | 001C004 | |
| DT- | SS | U007p001C004p001G007p000G004p000G007p000A004p000A007p0 | 1303 |
| 002167 | | 00A004p000U007p000U004p000U007p000G004p000C007p000U004 | |
| | | p0000007p0000004p000U007p001G004p001U007p000X2140 | |
| | AS | X033U1027p001C007p001A004p000A007p000A004p000A007p000G | 1304 |
| | | 004p000C007p000A004p000A007p000A004p000U007p000U004p00 | |
| | | OU007p000C004p000C007p000C004p000G007p000A004p001G004p | |
| | | 001U004 | |
| DT- | SS | G007p001A004p001A007p000A004p000U007p000U004p000U007p0 | 1305 |
| 002168 | | 00G004p000C007p000U004p000U007p000U004p000U007p000G004 | |
| | | p000C007p000C004p000A007p001A004p001A007p000X2140 | |
| | AS | X033U1027p001U007p001U004p000G007p000G004p000C007p000A | 1306 |
| | | 004p000A007p000A004p000A007p000G004p000C007p000A004p00 | |
| | | 0A007p000A004p000U007p000U004p000U007p000C004p001C004p | |
| | | 001C004 | |
| DT- | SS | C007p001U004p001U007p000C004p000G007p000G004p000C007p0 | 1307 |
| 002169 | | 00G004p000G007p000U004p000U007p000U004p000G007p000G004 | |
| | | p000A007p000U004p000A007p001U004p001U007p000X2140 | |
| | AS | X033A1027p001A007p001U004p000A007p000U004p000C007p000C | 1308 |
| | | 004p000A007p000A004p000A007p000C004p000C007p000G004p00 | |
| | | 0C007p000C004p000G007p000A004p000A007p000G004p001C004p | |
| | | 001G004 | |
| DT- | SS | U007p001C004p001G007p000G004p000C007p000G004p000G007p0 | 1309 |
| 002210 | | 000004p000U007p000U004p000G007p000G004p000A007p000U004 | |
| | | p000A007p000U004p000U007p0010004p001A007p000X2140 | |
| | AS | X033U1027p001A007p001A004p000A007p000U004p000A007p000U | 1310 |
| | | 004p000C007p000C004p000A007p000A004p000A007p000C004p00 | |
| | | 0C007p000G004p000C007p000C004p000G007p000A004p001A004p | |
| | | 001G004 | |
| DT- | SS | C007p001G004p001G007p000U004p000U007p000U004p000G007p0 | 1311 |
| 002211 | | 00G004p000A007p000U004p000A007p000U004p000U007p000U004 | |
| | | p000A007p000U004p000U007p001G004p001A007p000X2140 | |
| | AS | X033U1027p001C007p001A004p000A007p000U004p000A007p000A | 1312 |
| | | 004p000A007p000U004p000A007p000U004p000C007p000C004p00 | |
| | | 0A007p000A004p000A007p000C004p000C007p000G004p001C004p | |
| | | 001C004 | |
| DT- | SS | G007p001U004p001U007p000U004p000G007p000G004p000A007p0 | 1313 |
| 002212 | | 000004p000A007p000U004p000U007p000U004p000A007p000U004 | |
| | | p0000007p000G004p000A007p001C004p001U007p000X2140 | |
| | AS | X033A1027p001G007p001U004p000C007p000A004p000A007p000U | 1314 |
| | | 004p000A007p000A004p000A007p000U004p000A007p000U004p00 | |
| | | 0C007p000C004p000A007p000A004p000A007p000C004p001C004p | |
| | | 001G004 | |
| DT- | SS | G007p001G004p001A007p000U004p000A007p000U004p000U007p0 | 1315 |
| 002213 | | 000004p000A007p000U004p000U007p000G004p000A007p000C004 | |
| | | p000C007p000U004p000C007p001G004p001U007p000X2140 | |
| | AS | X033A1027p001C007p001G004p000A007p000G004p000G007p000U | 1316 |
| | | 004p000C007p000A004p000A007p000U004p000A007p000A004p00 | |
| | | 0A007p000U004p000A007p000U004p000C007p000C004p001A004p | |
| | | 001A004 | |
| DT- | SS | G007p001A004p001U007p000A004p000U007p000U004p000U007p0 | 1317 |
| 002214 | | 00A004p000U007p000U004p000G007p000A004p000C007p000C004 | |
| | | p0000007p000C004p000G007p001U004p001A007p000X2140 | |
| | AS | X033U1027p001A007p001C004p000G007p000A004p000G007p000G | 1318 |
| | | 004p000U007p000C004p000A007p000A004p000U007p000A004p00 | |
| | | 0A007p000A004p000U007p000A004p000U007p000C004p001C004p | |
| | | 001A004 | |
| DT- | SS | C007p001C004p001C007p000A004p000A007p000U004p000C007p0 | 1319 |
| 002215 | | 00C004p000A007p000C004p000G007p000U004p000U007p000U004 | |
| | | p0000007p000G004p000G007p001A004p001U007p000X2140 | |
| | AS | X033A1027p001U007p001C004p000C007p000A004p000A007p000A | 1320 |
| | | 004p000A007p000C004p000G007p000U004p000G007p000G004p00 | |
| | | 0A007p000U004p000U007p000G004p000G007p000G004p001U004p | |
| | | 001G004 | |
| DT- | SS | A007p001U004p001C007p000C004p000A007p000C004p000G007p0 | 1321 |
| 002216 | | 000004p000U007p000U004p000U007p000G004p000G007p000A004 | |
| | | p0000007p000G004p000C007p001A004p001U007p000X2140 | |
| | AS | X033A1027p001U007p001G004p000C007p000A004p000U007p000C | 1322 |
| | | 004p000C007p000A004p000A007p000A004p000A007p000C004p00 | |
| | | 0G007p000U004p000G007p000G004p000A007p000U004p001U004p | |
| | | 001G004 | |
| DT- | SS | C007p001C004p001A007p000C004p000G007p000U004p000U007p0 | 1323 |
| 002217 | | 00U004p000U007p000G004p000G007p000A004p000U007p000G004 | |
| | | p000C007p000A004p000C007p001U004p001U007p000X2140 | |
| | AS | X033A1027p001A007p001G004p000U007p000G004p000C007p000A | 1324 |
| | | 004p0000007p000C004p000C007p000A004p000A007p000A004p00 | |
| | | 0A007p000C004p000G007p000U004p000G007p000G004p001A004p | |
| | | 001U004 | |
| DT- | SS | C007p001G004p001U007p000U004p000U007p000U004p000G007p0 | 1325 |
| 002218 | | 00G004p000A007p000U004p000G007p000C004p000A007p000C004 | |
| | | p0000007p000G004p000A007p001G004p001A007p000X2140 | |
| | AS | X033U1027p001C007p001U004p000C007p000A004p000G007p000U | 1326 |
| | | 004p000G007p000C004p000A007p000U004p000C007p000C004p00 | |
| | | 0A007p000A004p000A007p000A004p000C007p000G004p001U004p | |
| | | 001G004 | |

X2140:

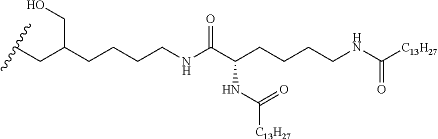

### Example 8. Target Engagement in Hemizygous DMSXL Mice

Myotonic dystrophy type 1 (DM1) is a genetic disorder characterized by progressive muscle wasting, myotonia, and cardiac conduction defects. DM1 is caused by a CTG nucleotide repeat expansion in the 3′UTR of the dystrophia myotonica protein kinase (DMPK) gene; the repeat can be present in from 50 to several thousand copies in DM1 patients. DMPK transcripts containing repeat expansions accumulate in the nucleus and sequester RNA-binding proteins such as MBNL1, which in turn results in disruption in the alternative splicing of certain genes.

Transgenic mouse models are utilized to investigate DM1 etiology and evaluate potential therapeutic strategies for the treatment of DM1. One such model is the DMSXL mouse model, a transgenic mouse model having a disease causing form of the DMPK gene containing over 1000 CTG repeats. Homozygous DMSXL mice harboring two copies of the mutant DMPK gene exhibit high mortality, growth inhibition, and muscle defects. Hemizygous DMSXL mice show very mild splice defects and, therefore, are more readily utilized to investigate agents designed to reduce the level of DMPK expression.

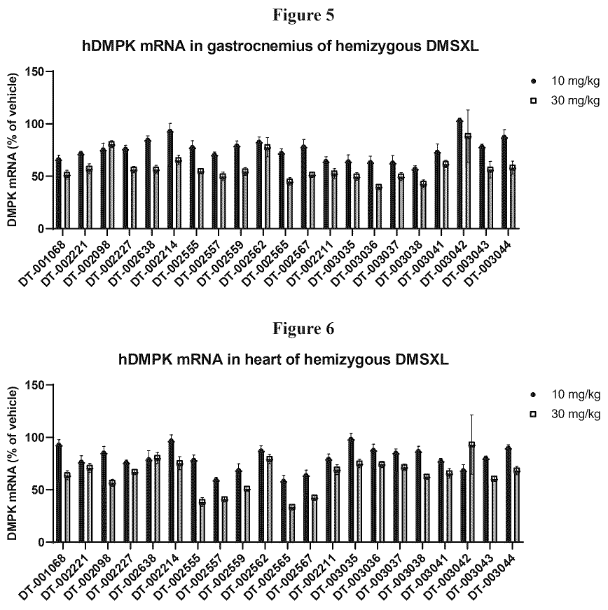

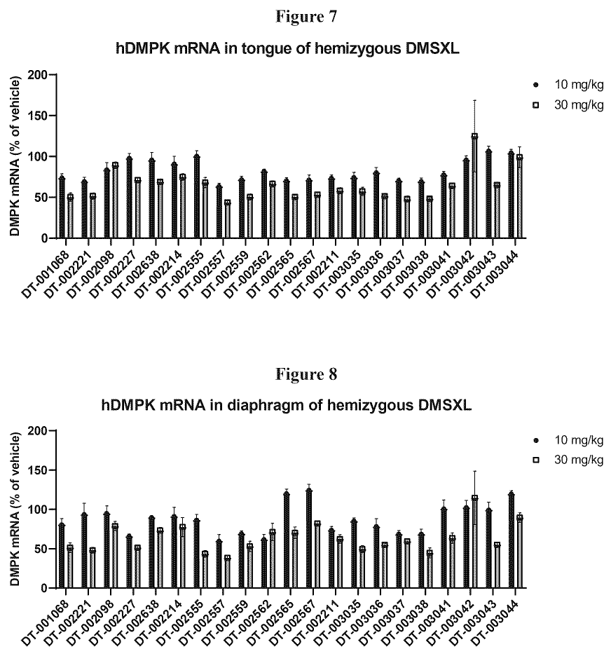

Conjugated siRNAs were evaluated for their ability to reduce human DMPK mRNA expression in hemizygous DMSXL mice. Groups of five mice each were administered a single dose of PBS or siRNA at a dose of 30 mg/kg. On Day 14, mice were sacrificed, and RNA was collected from gastrocnemius, heart, diaphragm, and tongue tissue for RNA extraction and quantitation of human DMPK mRNA levels by quantitative RT-PCR as described herein. The average percentage expression in each of the four collected muscle tissues was calculated for each treatment and is shown in Table 10. As illustrated in Table 10, the different compounds exhibited varying amounts of DMPK mRNA suppression (FIGS. 5-8).

TABLE 10
DMPK mRNA expression in muscle tissues of DMSXL mice
14 days following administration of 30 mg/kg of siRNA

| siRNA | Gastroc | | Heart | | Diaph | | Tongue | |
|---|---|---|---|---|---|---|---|---|
| Compound ID | Mean | SEM | Mean | SEM | Mean | SEM | Mean | SEM |
| DT-001068 | 53.7 | 4.1 | 80.0 | 6.6 | 59.6 | 8.8 | 57.6 | 6.2 |
| DT-002214 | 58.9 | 6.3 | 60.0 | 5.9 | 55.8 | 10.1 | 80.7 | 4.6 |
| DT-002046 | 61.2 | 0.6 | 70.5 | 5.6 | 80.0 | 9.9 | 90.9 | 12.3 |
| DT-002218 | 67.5 | 5.3 | 73.2 | 4.7 | 67.5 | 8.9 | 85.5 | 7.3 |
| DT-002211 | 74.7 | 5.7 | 73.9 | 4.2 | 60.5 | 3.5 | 59.9 | 7.5 |
| DT-002041 | 77.0 | 5.6 | 102.4 | 7.7 | 42.8 | 2.8 | 88.5 | 7.2 |
| DT-002101 | 77.0 | 4.8 | 88.6 | 10.3 | 91.0 | 7.9 | 71.7 | 6.1 |
| DT-002165 | 77.2 | 8.3 | 94.0 | 7.3 | 84.4 | 15.6 | 93.4 | 8.3 |
| DT-002164 | 79.3 | 5.4 | 84.6 | 6.3 | 97.0 | 7.6 | 84.8 | 9.7 |
| DT-002213 | 80.2 | 8.3 | 84.3 | 6.9 | 72.5 | 5.3 | 78.7 | 5.7 |
| DT-002098 | 80.9 | 7.6 | 109.2 | 7.5 | 83.0 | 12.9 | 68.0 | 4.7 |
| DT-002215 | 82.8 | 10.7 | 83.7 | 4.4 | 80.5 | 16.5 | 100.8 | 8.3 |
| DT-002212 | 85.7 | 4.3 | 82.7 | 3.3 | 59.8 | 5.3 | 68.8 | 6.2 |
| DT-002044 | 86.6 | 4.5 | 79.4 | 9.8 | 89.5 | 13.3 | 100.5 | 13.2 |
| DT-002102 | 88.2 | 14.9 | 106.0 | 10.4 | 103.2 | 12.4 | 77.6 | 4.4 |
| DT-002045 | 88.9 | 6.5 | 92.7 | 7.9 | 122.6 | 17.3 | 114.5 | 7.3 |
| DT-002043 | 89.1 | 8.8 | 104.1 | 10.0 | 114.0 | 17.0 | 105.5 | 7.3 |
| DT-002210 | 89.5 | 13.7 | 80.5 | 3.2 | 74.9 | 18.4 | 73.8 | 3.4 |
| DT-002099 | 94.2 | 10.1 | 132.6 | 9.1 | 180.9 | 39.8 | 101.5 | 8.0 |
| DT-002100 | 94.9 | 11.9 | 90.6 | 8.8 | 96.5 | 5.4 | 67.1 | 2.9 |
| DT-002167 | 95.2 | 12.0 | 92.2 | 8.3 | 119.9 | 7.2 | 106.7 | 8.7 |
| DT-002039 | 95.7 | 10.6 | 103.5 | 3.3 | 64.7 | 5.7 | 103.5 | 8.5 |
| DT-002097 | 100.1 | 14.8 | 134.4 | 12.1 | 110.3 | 21.4 | 93.1 | 9.5 |
| DT-002042 | 103.6 | 7.0 | 102.9 | 7.9 | 56.8 | 5.1 | 106.0 | 7.6 |
| DT-002040 | 105.9 | 14.3 | 88.2 | 6.3 | 80.4 | 9.1 | 86.4 | 9.4 |
| DT-002217 | 105.9 | 7.5 | 94.7 | 2.7 | 73.1 | 8.6 | 98.9 | 3.5 |
| DT-002216 | 106.7 | 7.5 | 71.8 | 4.2 | 86.2 | 6.4 | 80.7 | 11.7 |
| DT-002169 | 111.3 | 14.6 | 88.3 | 4.5 | 148.2 | 7.8 | 87.6 | 12.1 |
| DT-002168 | 112.2 | 11.7 | 108.6 | 7.3 | 116.6 | 11.8 | 105.9 | 6.0 |
| DT-002166 | 114.8 | 13.0 | 104.3 | 6.5 | 111.5 | 13.7 | 131.9 | 15.6 |

Compounds with activity comparable to that of DT-001068 (siRNA of SEQ ID NO: 1267 (SS) and SEQ ID NO: 1268 (AS)) were selected for further chemical modification (Table 11) and tested in DMSXL mice (Table 12).

### Example 9. Chemical Modification Variants of Active siRNAs

To determine whether variations in siRNA nucleotide sequence and/or modified nucleotide pattern would yield compounds with improved properties such as potency and duration of action, additional compounds were designed to introduce variations in the positions of chemical modifications into selected compounds. As DT-001068 was identified as a compound as suitably efficacious in the mouse model, compounds with activity comparable to that of DT-001068 were selected for further chemical modification.

The structure of each compound are shown in Table 11. Each antisense strand is 100% complementary to an equal length region of the nucleotide sequence of the human DMPK mRNA (NCBI Reference Sequence NM_001081563.2, deposited with GenBank on Jan. 4, 2014; SEQ ID NO: 1). The 5′ and 3′ nucleotide positions of each region are shown in Table 1, with “Start” and “End” corresponding to the 5′ and 3′ nucleotide positions, respectively, of the nucleotide sequence of the SEQ ID NO: 1. Each sense and antisense strand is assigned a unique “Strand ID”; for each siRNA, the sense strand comprises the C₇OH-DTx-01-32 conjugate and the antisense strand comprises the 5′-VP modification. Chemical modification notation is as described for the compounds in Table 9 and Table 3.

TABLE 11
Chemical variants of highly active compounds

| | | | SEQ |
|---|---|---|---|
| SIRNA | | | ID |
| ID | strand | Modified sequence | No. |
| DT- | SS | C004p001C007p001U007p000C004p000G007p000G004p00 | 1327 |
| 002221 | | OU007p000A004p000U007p000U004p000U007p000A004p0 | |
| | | 000007p000U004p000G007p000U004p000C007p000U004p | |
| | | 000G007p001U004p001U007p000X2140 | |
| | AS | X033A1027p001A007p001C004p000A007p000G004p000A0 | 1328 |
| | | 07p000C004p000A007p000A004p000U007p000A004p000A | |
| | | 007p000A004p000U007p000A004p000C007p000C004p000 | |
| | | G007p000A004p000G007p000G004p001A004p001A004 | |
| DT- | SS | G004p001G004p001U004p000C004p000A007p000U004p00 | 1329 |
| 002227 | | OG007p000G007p000A007p000G004p000U004p000A004p0 | |
| | | 000004p0000004p000A004p000C004p000G004p001U004p | |
| | | 0010004p000X2140 | |
| | AS | X033A1027p001A007p001C004p000G004p0000004p000A0 | 1330 |
| | | 07p000A004p0000004p000A004p000C004p000U004p000C | |
| | | 004p000C004p000A007p000U004p000G007p000A004p000 | |
| | | C004p000C004p001A004p001G004 | |
| DT- | SS | G004p001G004p0010004p000C004p000A007p0000004p00 | 1331 |
| 002638 | | 0G007p000G007p000A007p000G004p000U004p000A004p0 | |
| | | 00U004p000U004p000A004p000C004p000G004p001U004p | |
| | | 0010004p000X2140 | |
| | AS | X033U1027p001A007p001C004p000G004p000U004p000A0 | 1332 |
| | | 07p000A004p000U004p000A004p000C004p000U004p000C | |
| | | 004p000C004p000A007p000U004p000G007p000A004p000 | |
| | | C004p000C004p001A004p001G004 | |
| DT- | SS | G004p001A004p001U004p000A004p000U007p000U004p00 | 1333 |
| 002555 | | 0U007p000A007p0000007p0000004p000G004p000A004p0 | |
| | | 00C004p000C004p000U004p000C004p000G004p0010004p | |
| | | 001A004p000X2140 | |
| | AS | X033U1027p001A007p001C004p000G004p000A004p000G0 | 1334 |
| | | 07p000G004p000U004p000C004p000A004p000A004p000U | |
| | | 004p000A004p000A007p000A004p000U007p000A004p000 | |
| | | U004p000C004p001C004p001A004 | |
| DT- | SS | G004p001G004p001A004p000U004p000A004p000U007p00 | 1335 |
| 002557 | | 0U004p0000007p000A007p0000007p0000004p000G004p0 | |
| | | 00A004p000C004p000C004p0000004p000C004p000G004p | |
| | | 001U004p001A004p000X2140 | |
| | AS | X033U1027p001A007p001C004p000G004p000A004p000G0 | 1336 |
| | | 07p000G004p000U004p000C004p000A004p000A004p000U | |
| | | 004p000A004p000A007p000A004p0000007p000A004p000 | |
| | | U004p000C004p000C004p001A004p001A004 | |
| DT- | SS | U004p001G004p001G004p000A004p000U004p000A004p00 | 1337 |
| 002559 | | QU007p0000004p000U007p000A007p000U007p000U004p0 | |
| | | 00G004p000A004p000C004p000C004p000U004p000C004p | |
| | | 000G004p001U004p001A004p000X2140 | |
| | AS | X033U1027p001A007p001C004p000G004p000A004p000G0 | 1338 |
| | | 07p000G004p000U004p000C004p000A004p000A004p000U | |
| | | 004p000A004p000A007p000A004p000U007p000A004p000 | |
| | | U004p000C004p000C004p000A004p001A004p001A004 | |
| DT- | SS | G004p001G004p001A004p000U004p000A004p000U004p00 | 1339 |
| 002562 | | 00007p0000004p000A007p0000007p0000007p000G004p0 | |
| | | 00A004p000C004p000C004p000U004p000C004p000G004p | |
| | | 000U004p001C004p001A004p000X2140 | |
| | AS | X033U1027p001G007p001A004p000C004p000G004p000A0 | 1340 |
| | | 07p000G004p000G004p000U004p000C004p000A004p000A | |
| | | 004p000U004p000A007p000A004p000A007p000U004p000 | |
| | | A004p000U004p000C004p000C004p001A004p001A004 | |
| DT- | SS | G004p001A004p001U004p000A004p000U007p000U004p00 | 1341 |
| 002565 | | QU007p000A007p0000007p000U004p000G004p000A004p0 | |
| | | 00C004p000C004p000U004p000C004p000G004p000U004p | |
| | | 000A004p000X2140 | |
| | AS | X033U1027p001A007p001C004p000G004p000A004p000G0 | 1342 |
| | | 07p000G004p000U004p000C004p000A004p000A004p000U | |
| | | 004p000A004p000A007p000A004p0000007p000A004p000 | |
| | | U004p000C004p001C004p001A004 | |
| DT- | SS | U004p001G004p001G004p000A004p000U004p000A004p00 | 1343 |
| 002567 | | 00007p0000004p0000007p000A007p000U007p0000004p0 | |
| | | 00G004p000A004p000C004p000C004p000U004p000C004p | |
| | | 000G004p000U004p000A004p000X2140 | |
| | AS | X033U1027p001A007p001C004p000G004p000A004p000G0 | 1344 |
| | | 07p000G004p000U004p000C004p000A004p000A004p000U | |
| | | 004p000A004p000A007p000A004p000U007p000A004p000 | |
| | | U004p000C004p000C004p000A004p001A004p001A004 | |
| DT- | SS | C004p001G004p001G004p000U004p000U007p000U004p00 | 1345 |
| 003035 | | 0G007p000G004p000A007p000U004p000A007p000U004p0 | |
| | | 000007p000U004p000A007p000U004p000U004p001G004p | |
| | | 001A004p000X2140 | |
| | AS | X033U1027p001C007p001A004p000A007p000U004p000A0 | 1346 |
| | | 07p000A004p000A007p000U004p000A007p000U004p000C | |
| | | 007p000C004p000A007p000A004p000A007p000C004p000 | |
| | | C004p000G004p001C004p001C004 | |
| DT- | SS | C004p001G004p001G004p000U004p000U007p000U004p00 | 1347 |
| 003036 | | 0G004p000G007p000A007p000U004p000A007p000U004p0 | |
| | | 000007p000U004p000A004p000U004p000U004p001G004p | |
| | | 001A004p000X2140 | |
| | AS | X033U1027p001C007p001A004p000A004p000U004p000A0 | 1348 |
| | | 07p000A004p000A004p000U004p000A007p000U004p000C | |
| | | 007p000C004p000A007p000A004p000A007p000C004p000 | |
| | | C004p000G004p001C004p001C004 | |
| DT- | SS | C004p001G004p001G004p000U004p0000007p0000004p00 | 1349 |
| 003037 | | 0G007p000G007p000A007p000U004p000A004p000U004p0 | |
| | | 000004p0000004p000A004p0000004p0000004p001G004p | |
| | | 001A004p000X2140 | |
| | AS | X033U1027p001C007p001A004p000A004p0000004p000A0 | 1350 |
| | | 07p000A004p000A004p000U004p000A004p000U004p000C | |
| | | 004p000C004p000A007p000A004p000A007p000C004p000 | |
| | | C004p000G004p001C004p001C004 | |
| DT- | SS | C004p001G004p001G004p000U004p000U007p000U004p00 | 1351 |
| 003038 | | 0G007p000G007p000A007p000U004p000A004p000U004p0 | |
| | | 000004p000U004p000A004p000U004p000U004p001G004p | |
| | | 001U004p000X2140 | |
| | AS | X033U1027p001C007p001A004p000A004p000U004p000A0 | 1352 |
| | | 07p000A004p000A004p000U004p000A004p000U004p000C | |
| | | 004p000C004p000A007p000A004p000A007p000C004p000 | |
| | | C004p000G004p001C004p001C004 | |
| DT- | SS | C007p001G004p001G007p000U004p000U007p000U004p00 | 1353 |
| 003041 | | 0G007p000G004p000A007p000U004p000A007p0000004p0 | |
| | | 000007p0000004p000A007p000U004p0000007p001G004p | |
| | | 0010007p000X2140 | |
| | AS | X033U1027p001C007p001A004p000A007p000U004p000A0 | 1354 |
| | | 07p000A004p000A007p000U004p000A007p000U004p000C | |
| | | 007p000C004p000A007p000A004p000A007p000C004p000 | |
| | | C007p000G004p001C004p001C004 | |
| DT- | SS | C007p001G004p001G007p0000004p000U007p0000004p00 | 1355 |
| 003042 | | OG007p000G004p000A007p000U004p000A007p000U004p0 | |
| | | 00U007p000U004p000A007p000U004p000U007p000G004p | |
| | | 0000007p000X2140 | |
| | AS | X033U1027p001C007p001A004p000A007p000U004p000A0 | 1356 |
| | | 07p000A004p000A007p000U004p000A007p000U004p000C | |
| | | 007p000C004p000A007p000A004p000A007p000C004p000 | |
| | | C007p000G004p001C004p001C004 | |
| DT- | SS | C004p001G004p001G004p000U004p0000007p0000004p00 | 1357 |
| 003043 | | 0G007p000G004p000A007p000U004p000A007p000U004p0 | |
| | | 000007p0000004p000A007p000U004p000U004p001G004p | |
| | | 001U004p000X2140 | |
| | AS | X033U1027p001C007p001A004p000A007p000U004p000A0 | 1358 |
| | | 07p000A004p000A007p000U004p000A007p000U004p000C | |
| | | 007p000C004p000A007p000A004p000A007p000C004p000 | |
| | | C004p000G004p001C004p001C004 | |
| DT- | SS | C004p001G004p001G004p000U004p000U007p000U004p00 | 1359 |
| 003044 | | OG007p000G004p000A007p000U004p000A007p000U004p0 | |
| | | 000007p0000004p000A007p000U004p000U004p000G004p | |
| | | 0000004p000X2140 | |
| | AS | X033U1027p001C007p001A004p000A007p000U004p000A0 | 1360 |
| | | 07p000A004p000A007p000U004p000A007p000U004p000C | |
| | | 007p000C004p000A007p000A004p000A007p000C004p001 | |
| | | C004p001G004 | |

X2140:

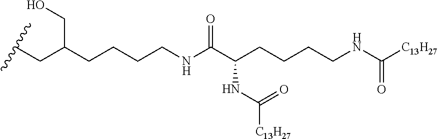

Selected compounds from Table 9 and all compounds from Table 11 were evaluated in hemizygous DMSXL mice. Groups of five DMSXL hemizygous mice each were treated with PBS or a single dose of PBS, 10 mg/kg, or 30 mg/kg of conjugated siRNA compound. At 28 days following injection, mice were sacrificed, and RNA was collected from gastrocnemius, heart, diaphragm, and tongue tissue for RNA extraction and quantitation of human DMPK mRNA levels by quantitative RT-PCR as described herein. The average percentage expression for human DMPK mRNA was calculated for each treatment. The data are shown in the following Tables 12 through 15 and in FIGS. 5 through 8.

TABLE 12
Human DMPK mRNA expression in gastrocnemius tissue of hemizygous
DMSXL mice 28 days following administration of siRNA

| | | |
|---|---|---|
| Compound | 10 mg/kg | 30 mg/kg |

| ID | Mean | SEM | Mean | SEM |
|---|---|---|---|---|
| DT-001068 | 66.0 | 4.1 | 51.5 | 4.3 |
| DT-002221 | 71.5 | 2.3 | 57.2 | 4.8 |
| DT-002098 | 75.3 | 6.4 | 80.7 | 3.3 |
| DT-002227 | 76.0 | 3.7 | 56.4 | 3.1 |
| DT-002638 | 84.5 | 4.2 | 56.4 | 4.0 |
| DT-002214 | 93.0 | 7.5 | 65.4 | 4.6 |
| DT-002555 | 77.5 | 6.5 | 54.9 | 2.2 |
| DT-002557 | 70.4 | 2.8 | 49.9 | 4.1 |
| DT-002559 | 79.0 | 4.8 | 54.7 | 3.7 |
| DT-002562 | 82.7 | 4.8 | 77.8 | 9.1 |
| DT-002565 | 72.0 | 4.2 | 45.1 | 3.6 |
| DT-002567 | 78.1 | 7.1 | 51.5 | 2.5 |
| DT-002211 | 64.3 | 4.4 | 52.8 | 4.6 |
| DT-003035 | 64.1 | 6.4 | 49.8 | 3.6 |
| DT-003036 | 63.3 | 6.0 | 39.9 | 2.7 |
| DT-003037 | 62.8 | 7.0 | 49.8 | 3.7 |
| DT-003038 | 57.1 | 2.9 | 42.8 | 3.8 |
| DT-003041 | 73.0 | 7.8 | 61.8 | 3.5 |
| DT-003042 | 103.3 | 2.3 | 88.4 | 25.0 |
| DT-003043 | 78.2 | 2.5 | 56.4 | 8.0 |
| DT-003044 | 87.3 | 7.3 | 58.3 | 6.3 |

TABLE 13
Human DMPK mRNA expression in heart tissue of hemizygous
DMSXL mice 28 days following administration of siRNA

| | | |
|---|---|---|
| Compound | 10 mg/kg | 30 mg/kg |

| ID | Mean | SEM | Mean | SEM |
|---|---|---|---|---|
| DT-001068 | 92.8 | 5.1 | 63.9 | 4.4 |
| DT-002221 | 76.4 | 6.1 | 71.0 | 4.4 |
| DT-002098 | 85.2 | 6.2 | 56.7 | 3.4 |
| DT-002227 | 75.9 | 2.5 | 67.0 | 2.3 |
| DT-002638 | 78.8 | 8.6 | 80.4 | 5.2 |
| DT-002214 | 96.7 | 5.8 | 75.4 | 6.3 |
| DT-002555 | 78.3 | 4.9 | 38.3 | 4.3 |
| DT-002557 | 59.3 | 2.7 | 41.1 | 1.9 |
| DT-002559 | 68.6 | 6.2 | 51.0 | 2.4 |
| DT-002562 | 87.2 | 4.8 | 79.4 | 4.6 |
| DT-002565 | 58.3 | 5.6 | 33.5 | 2.8 |
| DT-002567 | 63.7 | 5.1 | 42.5 | 2.0 |
| DT-002211 | 79.3 | 4.9 | 69.3 | 4.8 |
| DT-003035 | 98.3 | 5.7 | 75.1 | 4.1 |
| DT-003036 | 87.7 | 5.9 | 74.3 | 3.1 |
| DT-003037 | 85.0 | 4.1 | 71.6 | 3.1 |
| DT-003038 | 86.7 | 4.8 | 62.6 | 2.1 |
| DT-003041 | 77.4 | 2.7 | 65.7 | 4.5 |
| DT-003042 | 68.4 | 5.7 | 93.2 | 28.3 |
| DT-003043 | 79.9 | 2.3 | 60.5 | 2.0 |
| DT-003044 | 89.8 | 3.2 | 68.4 | 4.0 |

TABLE 14
Human DMPK mRNA expression in tongue tissue of hemizygous
DMSXL mice 28 days following administration of siRNA

| | | |
|---|---|---|
| Compound | 10 mg/kg | 30 mg/kg |

| ID | Mean | SEM | Mean | SEM |
|---|---|---|---|---|
| DT-001068 | 73.7 | 5.2 | 50.3 | 5.1 |
| DT-002221 | 69.3 | 5.2 | 51.5 | 3.8 |
| DT-002098 | 83.6 | 9.0 | 89.3 | 4.0 |
| DT-002227 | 97.8 | 5.9 | 71.1 | 2.8 |
| DT-002638 | 95.6 | 9.5 | 68.9 | 2.2 |
| DT-002214 | 90.8 | 9.5 | 74.9 | 4.0 |
| DT-002555 | 100.2 | 6.8 | 68.1 | 6.3 |
| DT-002557 | 63.2 | 4.0 | 43.8 | 2.0 |
| DT-002559 | 71.6 | 4.2 | 50.4 | 3.6 |
| DT-002562 | 81.5 | 3.1 | 66.5 | 3.8 |
| DT-002565 | 70.2 | 3.9 | 50.4 | 2.4 |
| DT-002567 | 71.0 | 6.4 | 53.3 | 2.5 |
| DT-002211 | 73.3 | 3.9 | 58.0 | 3.7 |
| DT-003035 | 74.0 | 6.5 | 57.3 | 5.1 |
| DT-003036 | 80.0 | 6.5 | 51.4 | 1.3 |
| DT-003037 | 70.2 | 3.2 | 47.8 | 3.7 |
| DT-003038 | 69.2 | 4.4 | 48.3 | 1.3 |
| DT-003041 | 77.4 | 4.4 | 64.0 | 3.3 |
| DT-003042 | 95.8 | 5.0 | 124.8 | 43.8 |
| DT-003043 | 106.6 | 5.9 | 65.1 | 1.3 |
| DT-003044 | 104.3 | 4.4 | 99.0 | 12.7 |

TABLE 15
Human DMPK mRNA expression in diaphragm tissue of hemizygous
DMSXL mice 28 days following administration of siRNA

| | | |
|---|---|---|
| Compound | 10 mg/kg | 30 mg/kg |

| ID | Mean | SEM | Mean | SEM |
|---|---|---|---|---|
| DT-001068 | 80.6 | 7.8 | 51.4 | 6.2 |
| DT-002221 | 93.6 | 14.1 | 48.1 | 3.7 |
| DT-002098 | 94.9 | 9.8 | 78.8 | 6.1 |
| DT-002227 | 65.7 | 3.2 | 51.4 | 2.2 |
| DT-002638 | 89.8 | 1.8 | 73.3 | 3.8 |
| DT-002214 | 91.1 | 11.6 | 77.5 | 12.3 |
| DT-002555 | 86.4 | 7.2 | 43.6 | 4.3 |
| DT-002557 | 59.8 | 8.3 | 38.4 | 2.6 |
| DT-002559 | 69.1 | 3.6 | 53.1 | 6.5 |
| DT-002562 | 61.7 | 6.7 | 71.3 | 11.1 |
| DT-002565 | 120.0 | 5.9 | 70.4 | 7.5 |
| DT-002567 | 124.4 | 7.6 | 82.0 | 3.0 |
| DT-002211 | 73.5 | 4.9 | 62.4 | 5.5 |
| DT-003035 | 85.2 | 4.2 | 49.5 | 4.4 |
| DT-003036 | 78.2 | 10.1 | 54.9 | 2.0 |
| DT-003037 | 68.5 | 4.9 | 59.4 | 2.7 |
| DT-003038 | 68.5 | 6.6 | 44.7 | 6.5 |
| DT-003041 | 100.8 | 11.3 | 63.6 | 6.7 |
| DT-003042 | 102.4 | 9.2 | 114.7 | 34.1 |
| DT-003043 | 99.4 | 9.9 | 55.0 | 3.4 |
| DT-003044 | 119.6 | 4.1 | 89.6 | 6.0 |

### Example 10. Evaluating Efficacy of Conjugated DMPK siRNAs in a Mouse Model of DM1

Transgenic mouse models are utilized to investigate DM1 etiology and evaluate potential therapeutic strategies for the treatment of DM1. One such model is the DMSXL mouse model, a transgenic mouse model having a disease causing form of the human DMPK gene containing over 1000 CTG repeats. Homozygous DMSXL mice harboring two copies of the mutant DMPK gene exhibit high mortality, growth inhibition, and muscle defects. Homozygous DMSXL mice were used to evaluate the ability of certain conjugated DMPK siRNAs to reduce the level of human DMPK mRNA. Groups of five DMSXL homozygous mice each were treated with PBS or a single dose of PBS or 30 mg/kg of conjugated siRNA compound. At 28 days following injection, mice were sacrificed, and RNA was collected from gastrocnemius, heart, diaphragm, and tongue tissue for RNA extraction and quantitation of human DMPK mRNA levels by quantitative RT-PCR as described herein. The average percentage expression for human DMPK mRNA was calculated for each treatment. The data are shown in the following Table 16.

TABLE 16
human DMPK mRNA levels in homozygous DMSXL mice
following administration of conjugated DMPK siRNA

| Compound | Gastrocnemius | | Heart | | Tongue | | Diaphragm | |
|---|---|---|---|---|---|---|---|---|
| ID | Mean | SEM | Mean | SEM | Mean | SEM | Mean | SEM |
| DT-001068 | 42.0 | 4.0 | 72.4 | 5.4 | 54.7 | 5.2 | 52.7 | 5.6 |
| DT-002221 | 61.9 | 3.4 | 88.1 | 2.6 | 74.6 | 7.5 | 63.6 | 6.8 |
| DT-002098 | 81.8 | 3.8 | 87.5 | 2.1 | 69.9 | 5.0 | 84.1 | 4.1 |
| DT-002227 | 59.2 | 2.8 | 85.0 | 2.8 | 69.6 | 3.7 | 64.1 | 3.5 |
| DT-002638 | 54.2 | 3.8 | 83.5 | 4.0 | 52.9 | 1.7 | 65.8 | 4.6 |
| DT-002214 | 47.6 | 2.0 | 89.5 | 1.9 | 76.8 | 4.2 | 77.4 | 3.0 |
| DT-002555 | 50.7 | 3.1 | 81.1 | 4.3 | 60.9 | 3.8 | 59.6 | 3.7 |
| DT-002557 | 37.9 | 4.4 | 71.5 | 6.2 | 41.3 | 2.3 | 54.8 | 7.4 |
| DT-002559 | 47.0 | 2.6 | 76.7 | 1.9 | 78.1 | 6.3 | 71.7 | 4.1 |
| DT-002562 | 41.5 | 2.0 | 90.3 | 2.8 | 76.3 | 3.4 | 76.0 | 2.1 |
| DT-002565 | 45.4 | 1.9 | 69.8 | 2.2 | 68.6 | 3.5 | 67.0 | 3.2 |
| DT-002567 | 44.3 | 2.0 | 68.5 | 2.3 | 67.6 | 3.5 | 54.8 | 4.4 |
| DT-002211 | 56.3 | 6.3 | 77.1 | 6.2 | 49.9 | 6.3 | 41.4 | 8.5 |
| DT-003035 | 46.7 | 3.3 | 67.1 | 2.3 | 42.3 | 2.6 | 49.8 | 2.7 |
| DT-003036 | 56.1 | 2.1 | 53.4 | 11.0 | 43.8 | 3.0 | 45.6 | 1.2 |
| DT-003037 | 38.8 | 0.6 | 67.5 | 3.6 | 47.2 | 3.6 | 47.4 | 4.0 |
| DT-003038 | 37.9 | 3.0 | 54.5 | 5.3 | 44.8 | 2.3 | 45.4 | 3.2 |
| DT-003041 | 45.7 | 3.1 | 67.0 | 3.7 | 54.0 | 1.9 | 54.6 | 1.3 |
| DT-003043 | 53.1 | 2.6 | 66.7 | 3.5 | 59.6 | 4.1 | 56.1 | 3.2 |

### Example 11. Detecting in Splicing Abnormalities

In DM 1, sequestration of RNA-binding proteins such as MBNL1 leads to numerous aberrant splicing events. One such aberrant splicing event is the inclusion of exon 11 of LIM domain binding 3 (LDB3) exon 11. Accordingly, the detection of LDB3 transcripts containing exon 11 may be used to evaluate the extent of splicing abnormalities in DM1 cells and tissues.

The inclusion of exon 11 in LDB3 was measured in homozygous DMSXL mice treated with conjugated DMPK siRNA. Groups of five mice each were administered a single dose of PBS or siRNA at a dose of 30 mg/kg. At 28 days, mice were sacrificed, and RNA was collected from multiple tissues for RNA extraction. Exon 11-containing LDB3 transcripts were measured as described herein; the data are shown in Table 17 as mean percent exon 11 inclusion relative to PBS treatment. As illustrated in Table 17, several conjugated DMPK siRNAs reduced the amount of exon 11-containing LDB3 transcripts, indicating a decrease in aberrant splicing activity in DMSXL mice.

TABLE 17
Detection of exon 11-containing LDB3 transcripts in homozygous
DMSXL mice following treatment with conjugated DMPK siRNA

| Compound | % exon 11 inclusion | |
|---|---|---|
| ID | compared to vehicle | SEM |
| DT-001068 | 68.7 | 5.6 |
| DT-002221 | 100.8 | 6.8 |
| DT-002098 | 100.0 | 6.3 |
| DT-002227 | 105.2 | 9.9 |
| DT-002638 | 106.1 | 6.3 |
| DT-002214 | 72.4 | 4.4 |
| DT-002555 | 82.8 | 5.5 |
| DT-002557 | 84.1 | 6.0 |
| DT-002559 | 70.2 | 4.7 |
| DT-002562 | 65.0 | 3.7 |
| DT-002565 | 80.2 | 6.2 |
| DT-002567 | 74.1 | 3.4 |
| DT-002211 | 71.9 | 5.9 |
| DT-003035 | 57.4 | 3.7 |
| DT-003036 | 59.8 | 5.1 |
| DT-003038 | 76.3 | 10.5 |

Compound DT-002211 showed favorable DMPK mRNA knockdown across muscle tissues in DMSXL mice and was selected as the parent compound for further studies.

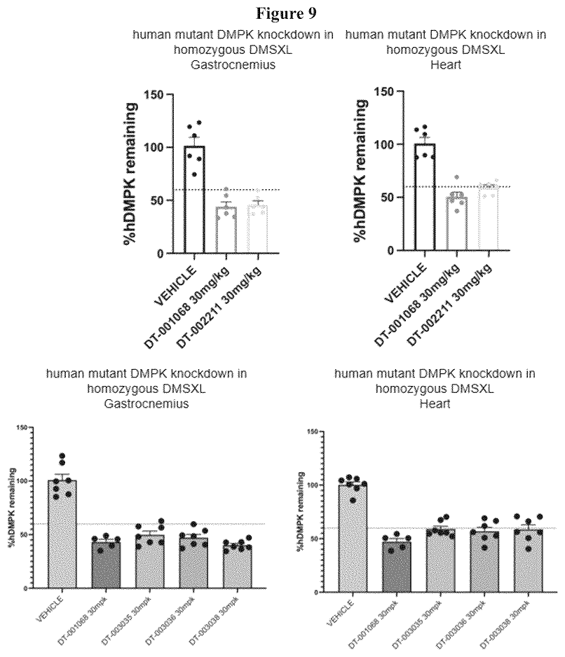

### Example 12. Target Engagement in Homozygous DMSXL (FIG. 9)

Homozygous DMSXL mice were used to evaluate the ability of certain conjugated DMPK siRNAs to reduce the level of human DMPK mRNA in a 90-day efficacy study. Groups of seven DMSXL homozygous mice each were treated with either PBS or 30 mg/kg of conjugated siRNA compound at Day 0, 30 and 59 (for a total of 3 doses) and sacrificed at Day 90. At the 90-day terminal time point, mice were sacrificed, and tissue samples were collected from gastrocnemius, heart, diaphragm, and tongue for RNA extraction and quantitation of human DMPK mRNA levels by quantitative RT-PCR as described herein. The average percentage expression for human DMPK mRNA was calculated for each treatment. The data are shown in the FIG. 9.

As shown in FIG. 9, several compounds showed at least 40% hDMPK mRNA reduction in disease relevant tissues in homozygous DMSXL mice.

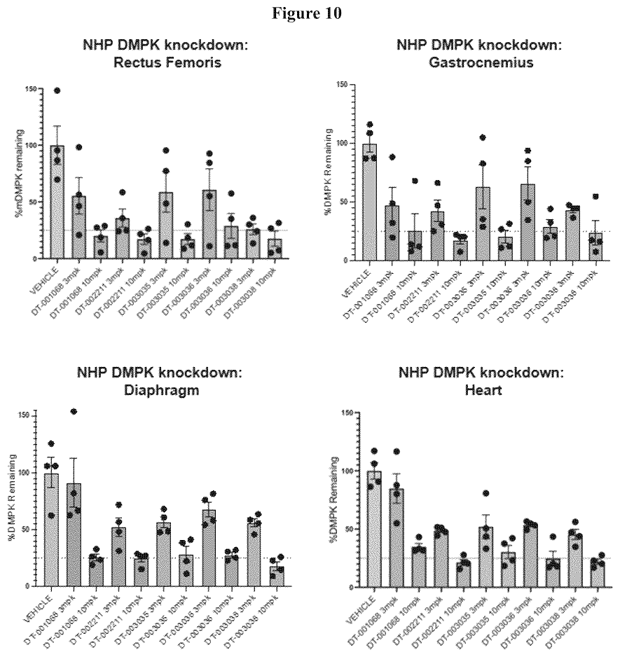

### Example 13. Target Engagement in NHP (FIG. 10)

The objectives of this study were to determine the pharmacodynamic (PD) characteristics of the conjugated siRNAs when given to cynomolgus monkeys, as a single dose via 1 hour intravenous (IV) infusion. The compounds were dosed once by IV infusion at both 3 mg/kg and 10 mg/kg on Day 1 and tissue samples were collected at Day 29. Target engagement data were determined by analyzing DMPK mRNA levels in selected tissues (rectus femoris, gastrocnemius, diaphragm and heart) collected at 29 days.

As shown in FIG. 10, several compounds showed approximately 75% DMPK mRNA knockdown in disease relevant tissues in NHP. For example, DT-003038 showed the most potent DMPK knockdown at the low dose (3 mg/kg) in the rectus femoris muscle.

Furthermore, DT-003038 showed extended target engagement across 3-6 months in disease-relevant tissues.

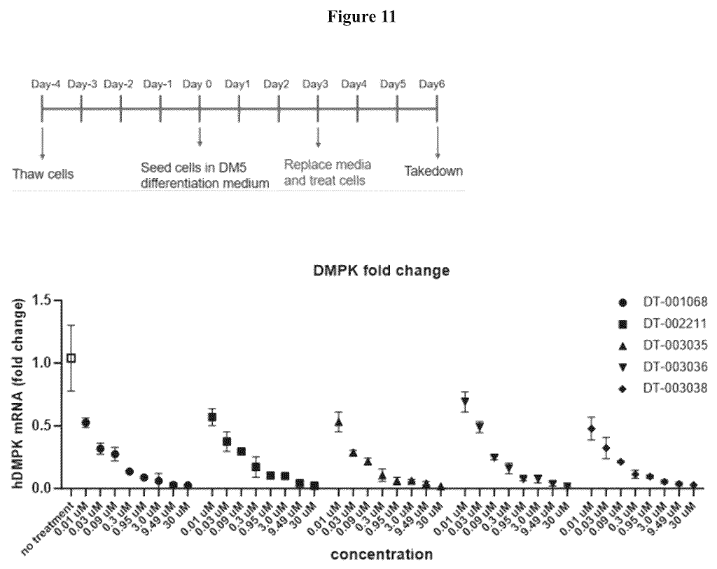

### Example 14. Target Engagement in DM1 Patient Myotubes (FIG. 11)

DM1 patient derived cells were cultured in differentiation media to form myotubes, and then treated with compounds for three days in serum free condition. On day three, cells were lysed and reverse transcription was done using cell lysates. qPCR reaction was run using DMPK Taqman probes to determine the mRNA level of DMPK. DMPK expression is normalized to internal control VPS26a to remove cell number variation.

All compounds of FIG. 11 showed potent, dose-dependent hDMPK mRNA knockdown in DM1 patient myotubes.

### Example 15. Efficacy (Splicing Correction) in Homozygous DMSXL

Homozygous DMSXL mice were used to evaluate the ability of certain conjugated DMPK siRNAs to correct the splicing defects in a 90-day efficacy study. Groups of seven DMSXL homozygous mice each were treated with either PBS or 30 mg/kg of conjugated siRNA compound at Day 0, 30 and 59 (for a total of 3 doses) and sacrificed at Day 90. At the 90-day terminal time point, mice were sacrificed, and tissue samples were collected from gastrocnemius, heart, diaphragm, and tongue for RNA extraction. For each tissue the composite splice index was measured as described in detail in the “RT-PCR analysis of gene splicing” (Example 3).

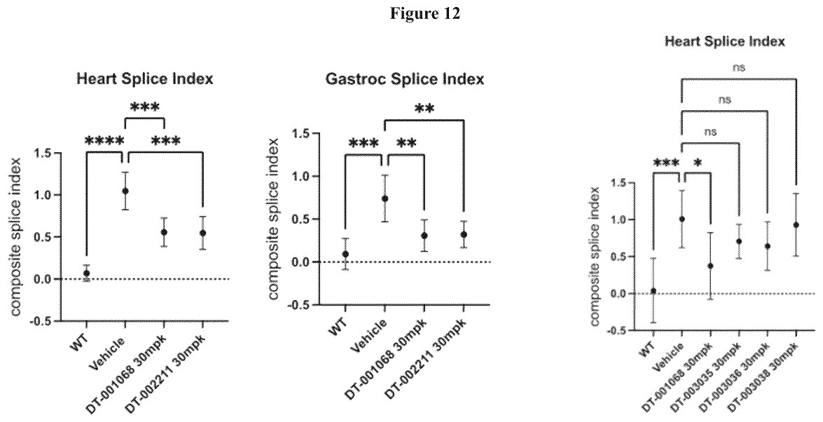

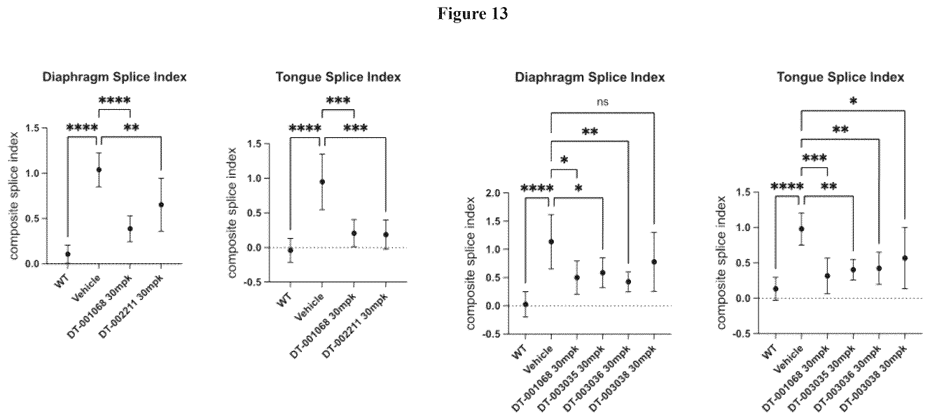

As shown in FIGS. 12 and 13, several compounds showed correction of composite splice index across disease-relevant tissues in homozygous DMSXL mice.

In FIGS. 12 and 13, the sources from which the composite scores were generated are listed below.

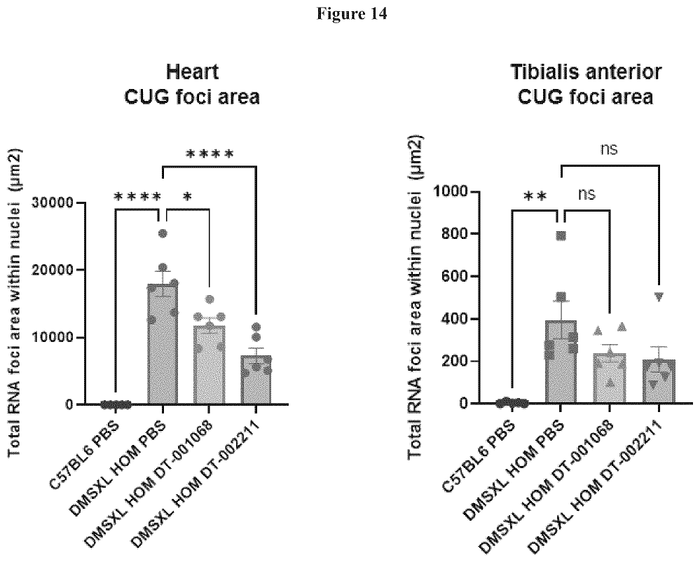

### Example 16. Efficacy (CUG Foci Reduction) in Homozygous DMSXL (FIG. 14)

Homozygous DMSXL mice were used to evaluate the ability of certain conjugated DMPK siRNAs to correct the splicing defects in a 90-day efficacy study. Groups of seven DMSXL homozygous mice each were treated with either PBS or 30 mg/kg of conjugated siRNA compound at Day 0, 30 and 59 (for a total of 3 doses) and sacrificed at Day 90. At the 90-day terminal time point, mice were sacrificed, and tissue samples were collected from heart and tibialis anterior. For each tissue, CUG foci were measured as described in detail in the “RNA in situ hybridization” (Example 3).

As shown in FIG. 14, the compound DT-002211 showed an average reduction of CUG foci in heart and tibialis anterior in homozygous DMSXL mice.

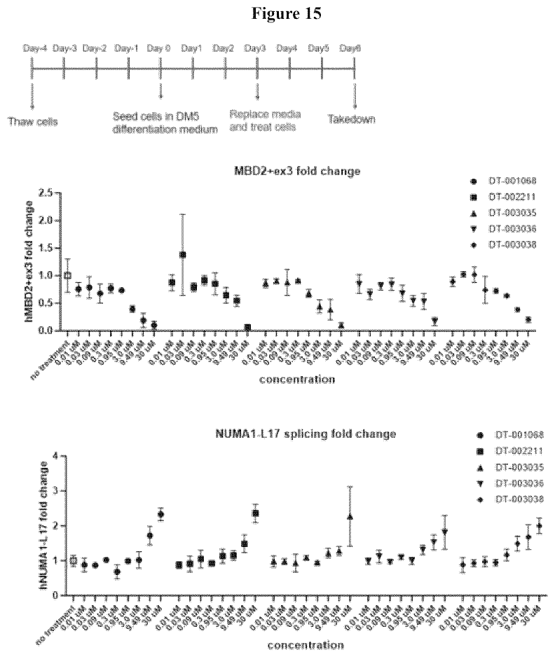

### Example 17. Efficacy (Splicing Correction) in DM1 Patient Myotubes (FIG. 15)

DM1 patient derived cells were cultured in differentiation media to form myotubes, and then treated with compounds for three days in serum free condition. On day three, cells were lysed and reverse transcription was done using cell lysates. DM1 patient derived cells were cultured in differentiation media to form myotubes, and then treated with compounds for three days in serum free condition. On day three, cells were lysed and reverse transcription was done using cell lysates. qPCR reaction was run against MBD2 and NUMA1 total gene and MBD2 ex3 and NUMA1 ex17 splice variant gene. Target gene expression is normalized to internal control VPS26a to remove cell number variation. Splice variant gene expression was then normalized to splice total gene expression to subtract the effect treatments have on total gene expression.

As shown in FIG. 15, several compounds showed a dose-dependent correction of individual splice events in DM1 patient myotubes.

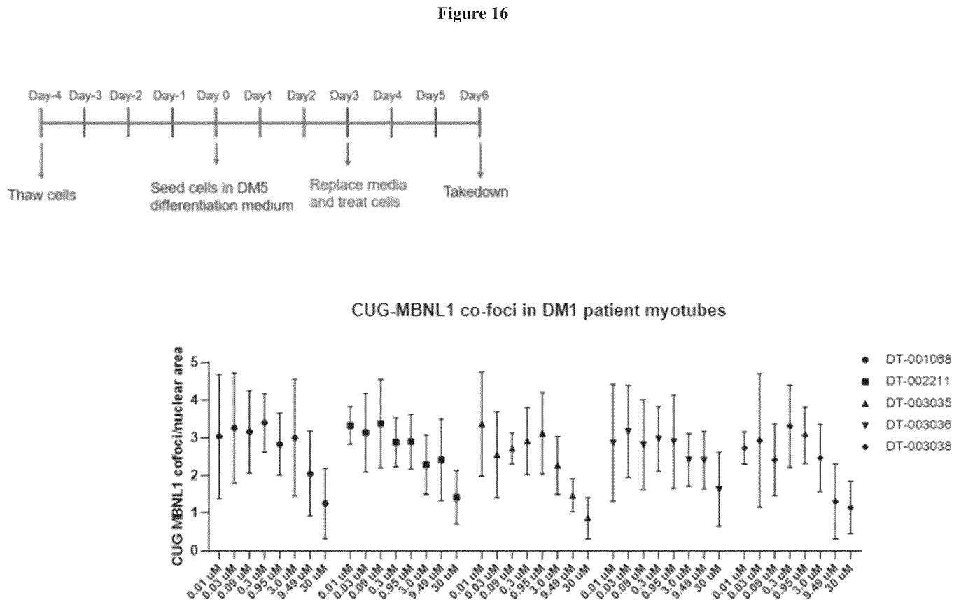

### Example 18. Efficacy (CUG Foci Reduction) in DM1 Patient Cells (FIG. 16)

After treatment, cells were fixed in 1% paraformaldehyde in DPBS for 20 min, incubated with 70% ethanol for 10 min. For hybridization, cells were incubated with hybridization buffer for 30 min at room temperature, then with 100 nM 2XPNA-(CAG)₅-Cy3 probe mix diluted in hybridization buffer, protected from light for 1.5 hours at 37° C., and 30 more minutes at 37° C. with hybridization buffer again. For primary antibody treatment, cells were incubated with blocking buffer for 1 hour at room temperature then with 1:200 MBNL1 Ab (SigmaMillipore #MABE70, Clone 4A8) diluted with blocking buffer overnight at 4° C., protected from light. For secondary antibody treatment, cells were incubated with 1:1000 DAPI and Alexa488 goat anti-mouse IgG DAPI in blocking buffer for 1 hr at RT. Cells were washed 3 times with PBS after each step. After final wash, plate is ready for imaging and can be stored in 50 uL PBS at 4° C.

As shown in FIG. 16, several compounds show a dose-dependent reduction of CUG-MBNL1 co-foci in DM1 patient myotubes.

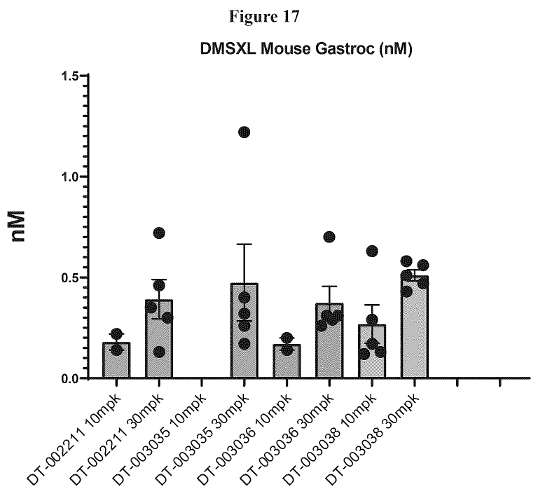

### Example 19. Tissue Exposure in DMSXL (FIG. 17)

Tissue exposure was measured in flash frozen tissue samples collected at study termination. Flash frozen tissues were homogenized at a concentration of 50 mg tissue/mL RIPA buffer (ThermoFisher, Catalog #89901). Homogenized samples were then run on a fit-for-purpose HELISA assay to determine the tissue concentration of DT-003038 detected. An 11-point standard curve consisting of a known concentration of DT-003038 was generated in blank tissue matrix comprised of pooled samples from untreated subjects. Standards and samples were annealed in a thermocycler (5 min at 90° C., 10 min gradient to 70° C., hold for 10 min, and a final hold at 25° C. for a minimum of 5 min) to custom biotinylated capture probe designed to hybridize to the guide strand of DT-003038. The resulting reaction product (40 μL/well) was then transferred as technical replicates to a streptavidin-coated MSD GOLD 96-well Small Spot Streptavidin SECTOR Plate (Mesoscale Diagnostics, Catalog #L⁴⁵SA-1) and incubated for 1 hr at 24° C. with shaking at 600 RPM. After washing the plate 3 times with MSD Wash Buffer (Mesoscale Diagnostics, Catalog #R61AA-1), samples were incubated with digoxin-conjugated detection probe for 1 hr at 24° C. with shaking at 600 RPM. The plate was washed again 3 times with MSD wash buffer on a plate washer, and incubated with sulfa-tagged digoxin antibody, comprised of Goat Anti-Digoxigenin/Digoxin (DIG) (Vector Labs, Catalog #MB-7000-1) conjugated to MSD GOLD SULFO-TAG NHS-Ester (Mesoscale Diagnostics, Catalog #R91AO-1), at a concentration of 1 μg/mL in 0.25% Triton X-100 in PBS for 1 hr at 24° C. with shaking at 600 RPM. The plate was then washed 3 times with MSD wash buffer on a plate washer, before 150 μL MSD Read Buffer A (Mesoscale Diagnostics, Catalog #R92TG-2) was added to the plate. The plate was read on a MESO QuickPlex SQ 120 Plate Reader (Mesoscale Diagnostics). A 4-Parameter Logistic regression curve was fit to the standard curve and used to extrapolate tissue concentrations in MSD Discovery Workbench (Mesoscale Diagnostics, LSR_4_0_13). All standards and quality control samples that fell within the lower limit of quantitation (LLOQ) and the upper limit of quantitation (ULOQ) had a coefficient of variation (CV) less than 20%. All values in DT-003038-treated samples fell within a 20% CV and were above LLOQ and below ULOQ. Averaged data from technical replicates were further averaged across biological replicates.

The compounds were detected to varying extents in gastrocnemius of homozygous DMSXL mice. For example, DT-003038 showed the strongest tissue exposure at the low dose (10 mg/kg) compared to the other compounds as shown in FIG. 17.

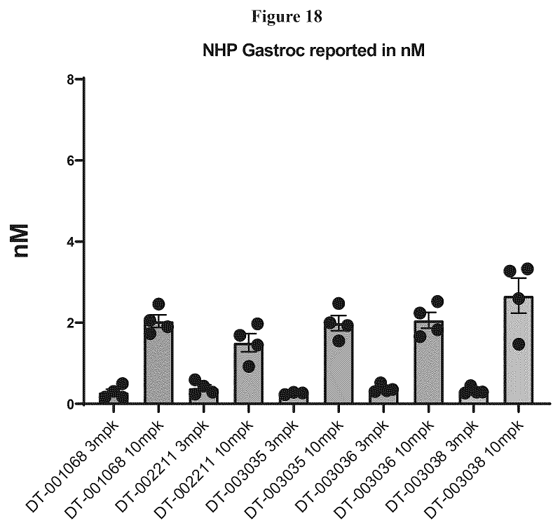

### Example 20. Tissue Exposure in NHP (FIG. 18)

Tissue exposure was measured in flash frozen tissue samples collected at study termination. Flash frozen tissues were homogenized at a concentration of 50 mg tissue/mL RIPA buffer (ThermoFisher, Catalog #89901). Homogenized samples and plasma were then run on a fit-for-purpose HELISA assay to determine the tissue concentration of DT-003038 detected. An 11-point standard curve consisting of a known concentration of DT-003038 was generated in blank tissue matrix comprised of pooled samples from untreated subjects. Samples were diluted up to 1000-fold in blank matrix to bring the concentration within the range of the assay. Standards and samples were annealed in a thermocycler (5 min at 90° C., 10 min gradient to 70° C., hold for 10 min, and a final hold at 25° C. for a minimum of 5 min) to custom biotinylated capture probe designed to hybridize to the guide strand of DT-003038. The resulting reaction product (40 μL/well) was then transferred as technical replicates to a streptavidin-coated MSD GOLD 96-well Small Spot Streptavidin SECTOR Plate (Mesoscale Diagnostics, Catalog #L45SA-1) and incubated for 1 hr at 24° C. with shaking at 600 RPM. After washing the plate 3 times with MSD Wash Buffer (Mesoscale Diagnostics, Catalog #R61AA-1), samples were incubated with digoxin-conjugated detection probe for 1 hr at 24° C. with shaking at 600 RPM. Plate was washed again 3 times with MSD wash buffer on a plate washer, and incubated with sulfa-tagged digoxin antibody, comprised of Goat Anti-Digoxigenin/Digoxin (DIG) (Vector Labs, Catalog #MB-7000-1) conjugated to MSD GOLD SULFO-TAG NHS-Ester (Mesoscale Diagnostics, Catalog #R91AO-1), at a concentration of 1 μg/mL in 0.25% Triton X-100 in PBS for 1 hr at 24° C. with shaking at 600 RPM. The plate was then washed 3 times with MSD wash buffer on a plate washer, before 150 μL MSD Read Buffer A (Mesoscale Diagnostics, Catalog #R92TG-2) was added to plate. The plate was read on a MESO QuickPlex SQ 120 Plate Reader (Mesoscale Diagnostics). A 4-Parameter Logistic regression curve was fit to the standard curve and used to extrapolate tissue concentrations in MSD Discovery Workbench (Mesoscale Diagnostics, LSR_4_0_13). All standards and quality control samples that fell within the lower limit of quantitation (LLOQ) and the upper limit of quantitation (ULOQ) had a coefficient of variation (CV) less than 20%. All values in DT-003038-treated samples fell within a 20% CV and were above LLOQ and below ULOQ. Averaged data from technical replicates were further averaged across biological replicates.

As shown in FIGS. 18, several compounds were detected at both doses in NHP gastrocnemius. For example, DT-003038 had higher tissue exposure on average compared to the other compounds.

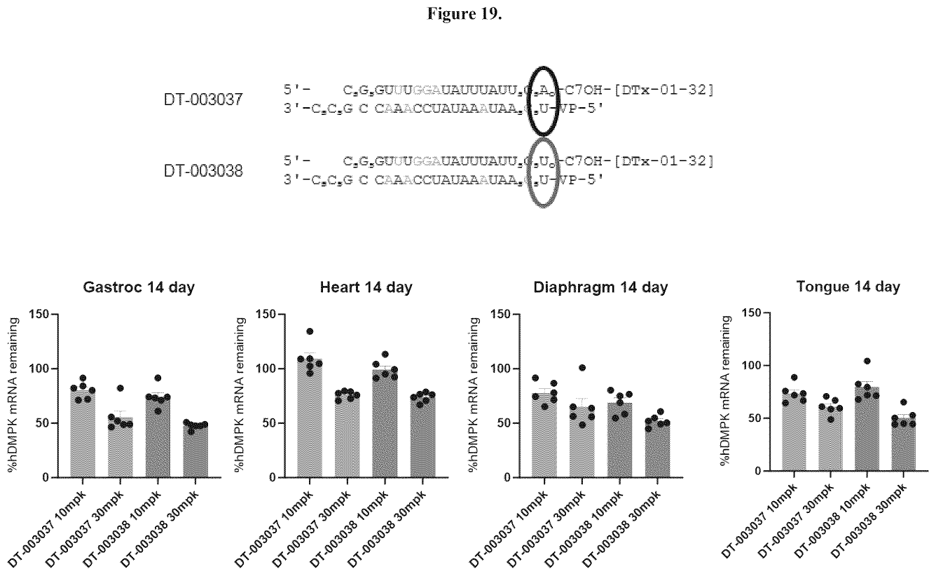

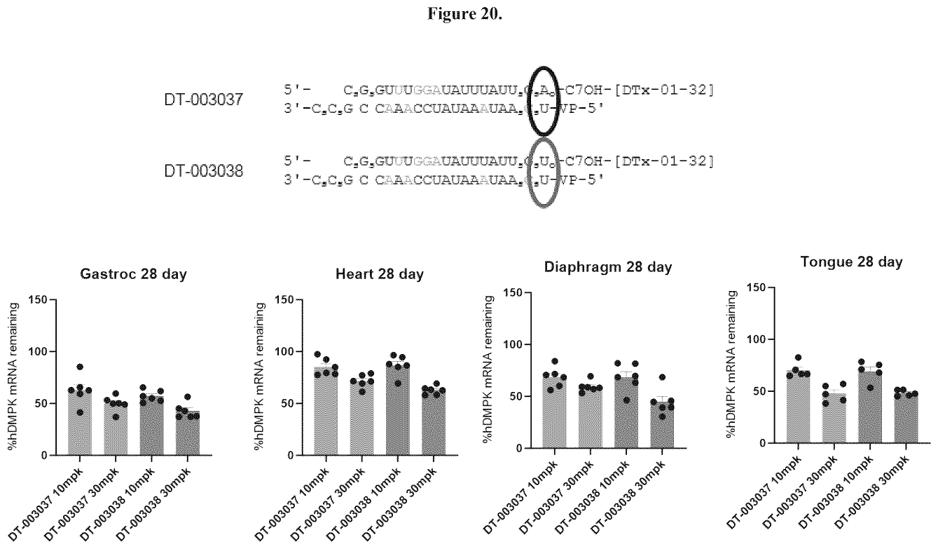

### Example 21. Effect of U—U Mismatch (FIGS. 19 and 20)

Studies in DMSXL mice were conducted to compare the effect of the DT-003038 UU mismatch. Compound DT-003038 was evaluated in 14-day and 28-day target engagement studies in hemizygous DMSXL at 10 mg/kg (IV) and 30 mg/kg (IV) versus the corresponding DT-003037 duplex with no mismatch.

The results are shown below in Tables 18 and 19 and FIGS. 19 and 20.

TABLE 18
human DMPK mRNA levels in DMSXL mice at 14 days following
administration of DT-003037 and DT-003038

| | | DT-003037 | | DT-003038 | |
|---|---|---|---|---|---|
| | | 10 mg/kg | 30 mg/kg | 10 mg/kg | 30 mg/kg |
| Gastroc | % KD | 19.8 | 44.3 | 25.8 | 47.6 |
| | SD | 7.7 | 13.4 | 9.9 | 2.9 |
| Heart | % KD | −9.2 | 24.2 | 0.8 | 26.2 |
| | SD | 13.2 | 3.6 | 8.4 | 4.4 |
| Diaphragm | % KD | 22.1 | 34.9 | 31.1 | 48.0 |
| | SD | 9.7 | 18.6 | 10.4 | 5.4 |
| Tongue | % KD | 26.4 | 39.0 | 20.4 | 49.7 |
| | SD | 8.6 | 7.6 | 13.3 | 8.2 |

TABLE 19
Human DMPK mRNA levels in DMSXL mice at 28 days following
administration of DT-003037 and DT-003038

| | | DT-003037 | | DT-003038 | |
|---|---|---|---|---|---|
| | | 10 mg/kg | 30 mg/kg | 10 mg/kg | 30 mg/kg |
| Gastroc | % KD | 37.2 | 50.2 | 42.9 | 57.2 |
| | SD | 14.0 | 7.4 | 5.7 | 7.5 |
| Heart | % KD | 15.0 | 28.4 | 13.3 | 37.4 |
| | SD | 8.3 | 6.3 | 9.6 | 4.2 |
| Diaphragm | % KD | 31.5 | 40.6 | 31.5 | 55.3 |
| | SD | 9.8 | 5.4 | 13.2 | 12.9 |
| Tongue | % KD | 29.8 | 52.2 | 30.8 | 51.7 |
| | SD | 7.2 | 8.2 | 9.8 | 2.9 |

DT-003038, which contains a UU mismatch, showed better potency in diaphragm and tongue than the corresponding DT-003037 with no mismatch at 30 mg/kg (IV) after 14 days, and better potency in diaphragm after 28 days.

1-204. (canceled)

205. dsRNAi agent of the structure of formula (II): 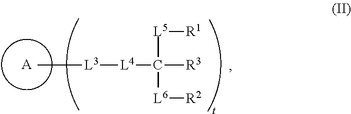
or a pharmaceutically acceptable salt thereof,
wherein A is a double stranded nucleic acid having suppressing activity of DMPK gene expression, comprising a sense strand and an antisense strand forming a duplex with the sense strand, wherein each of the antisense strand and the sense strand is independently 10 to 30 nucleotides in length,
L³ is independently selected from:
(i) a bond; 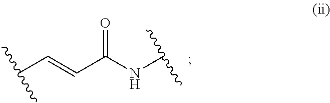 [SMILES: */C=C/C(=O)N*]
(iii) —OP(O)(OH)O—;
(iv) —OP(S)(OH)O—; and
(v) —O—;
L⁴ is independently selected from:
(i) substituted or unsubstituted 2 to 20 membered heteroalkylene;
(ii) -L⁷-NH—C(O)— or -L⁷-C(O)—NH—, wherein L⁷ is substituted or unsubstituted C1-C20 alkylene; 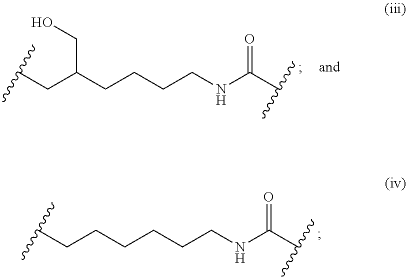 [SMILES: *CC(CO)CCCCNC(*)=O ; *CCCCCCNC(*)=O]
L⁵ is substituted or unsubstituted 2 to 20 membered heteroalkylene, or -L^{5A}L^{5B}L^{5C}L^{5D}L^{5E}-, wherein L^{5A} is independently unsubstituted C₁-C₈ alkylene; L^{5B} is independently —NHC(O)—; L^{5C} is independently a bond; L^{5D} is independently a bond or unsubstituted C₁-C₈ alkylene; and L^{5E} is independently a bond or —NHC(O)—;
L⁶ is —NHC(O)—;
R¹ and R² are independently unsubstituted C₁-C₁₉ alkyl or unsubstituted C₁-C₁₉ alkenyl;
R³ is hydrogen; and
t is 1.

206. The dsRNAi agent of claim 205, or a pharmaceutically acceptable salt thereof, wherein A is a double stranded nucleic acid comprising a sense strand and an antisense strand, the antisense strand forming a duplex with the sense strand, wherein each of the antisense strand and the sense strand is independently 17 to 25 nucleotides in length, wherein the nucleotide sequence of the antisense strand is at least 90% complementary to SEQ ID NO: 1, and the nucleotide sequence of the sense strand has no more than two mismatches to the nucleotide sequence of the antisense strand.

207. The dsRNAi agent of claim 205, or a pharmaceutically acceptable salt thereof, wherein:
the sense strand has a nucleic acid sequence that is at least about 95% identical to SEQ ID NO: 1, as measured over the entirety of the sense strand; and/or
the antisense strand comprises a nucleic acid sequence that is at least about 95% complementary to SEQ ID NO: 1, as measured over the entirety of the antisense strand.

208. The dsRNAi agent of claim 205, or a pharmaceutically acceptable salt thereof, wherein:
the antisense strand comprises a nucleic acid sequence that is 100% complementary to SEQ ID NO: 1, as measured over the entirety of the antisense strand; and/or
the nucleic acid sequence of the sense strand has no more than one mismatch to the nucleic acid sequence of the antisense strand.

209. The dsRNAi agent of claim 205, or a pharmaceutically acceptable salt thereof, wherein L³ is attached to a 3′ carbon of a 3′ terminal nucleotide of the sense strand of the double stranded nucleic acid.

210. The dsRNAi agent of claim 205, or a pharmaceutically acceptable salt thereof, wherein L³ is independently —OPO₂—O— or —O—.

211. The dsRNAi agent of claim 205, or a pharmaceutically acceptable salt thereof,
wherein L⁴ is independently 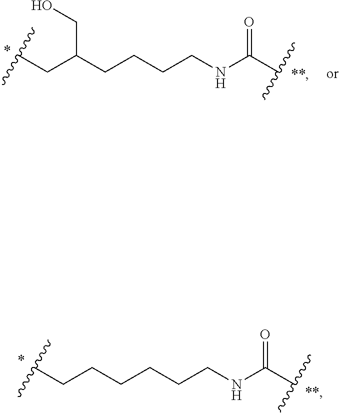 [SMILES: *CC(CO)CCCCNC(*)=O ; *CCCCCCNC(*)=O]
wherein * indicates the point of attachment to L³ and ** indicates the point of attachment to C.

212. The dsRNAi agent of claim 205, or a pharmaceutically acceptable salt thereof,
wherein -L³-L⁴- is independently 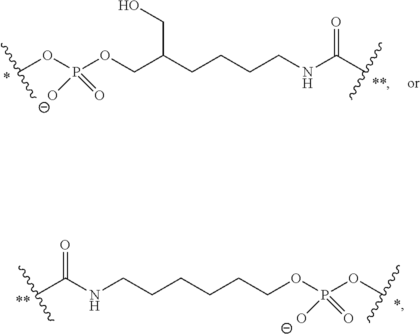 [SMILES: *OP(=O)([O-])OCC(CO)CCCCNC(*)=O ; *OP(=O)([O-])OCCCCCCNC(*)=O]
wherein * indicates the point of attachment to the double stranded nucleic acid and ** indicates the point of attachment to C.

213. The dsRNAi agent of claim 205, or a pharmaceutically acceptable salt thereof, of formula (Id) 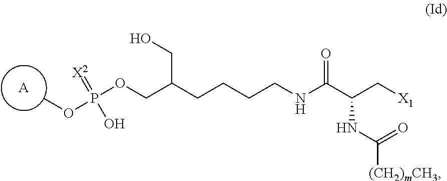
wherein
X² is selected from O and S;
X¹ is 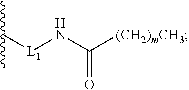
L¹ is —(CH₂)ₙ—, —(CH₂)ₙL²(CH₂)ₙ—, or a bond;
L² is —C(═O)NH—, —C(═O)O—, —OC(═O)O—, —NHC(═O)O—, —NHC(═O)NH—, —C(═S)NH—, —C(═O)S—, —NH—, O (oxygen), or S (sulfur), and wherein each m is independently an integer from 10 to 18 and wherein each n is independently an integer from 1 to 6; and
A is defined according to any one of the preceding claims.

214. The dsRNAi agent of claim 213, or a pharmaceutically acceptable salt thereof, wherein each m is 12, L₁ is —(CH₂)ₙ—, and n is 3.

215. The dsRNAi agent of claim 205, or a pharmaceutically acceptable salt thereof, wherein:
the nucleotide sequence of the antisense strand comprises at least 19 contiguous nucleotides of a nucleotide sequence selected from any one of SEQ ID Nos: 294 to 584; and/or
the nucleotide sequence of the sense strand comprises at least 17 contiguous nucleotides of a nucleotide sequence selected from any one of SEQ ID Nos: 3 to 293.

216. The dsRNAi agent of claim 205, or a pharmaceutically acceptable salt thereof, wherein the antisense strand is 21 nucleotides in length, the sense strand is 19 nucleotides in length, the duplex is 19 nucleotide pairs in length, and the nucleotide sequence of the antisense strand is at least about at least about 95% complementary to the nucleotide sequence of the sense strand in the duplex.

217. The dsRNAi agent of claim 205, or a pharmaceutically acceptable salt thereof, wherein:
the nucleotide sequence of the antisense strand is identical to a nucleotide sequence selected from any one of SEQ ID Nos: 294 to 584; and/or
the nucleotide sequence of the sense strand is identical to a nucleotide sequence selected from any one of SEQ ID Nos: 3 to 293.

218. The dsRNAi agent of claim 205, or a pharmaceutically acceptable salt thereof, wherein the double stranded nucleic acid comprises
(i) an antisense strand selected from SEQ ID Nos: 294 to 584, and
(ii) a sense strand selected from SEQ ID Nos: 3 to 293.

219. The dsRNAi agent of claim 205, or a pharmaceutically acceptable salt thereof, wherein at least one nucleotide of the sense strand and the antisense strand is a modified nucleotide; and the modified nucleotide comprises one or more of a modified sugar moiety, a modified internucleoside linkage, and a 5′-terminal modified phosphate group.

220. The dsRNAi agent of claim 219, or a pharmaceutically acceptable salt thereof, wherein the modified nucleotide comprises one or more of the following:
the modified nucleotide comprising a modified sugar moiety is selected from a 2′-fluoro nucleotide, a 2′-O-methyl nucleotide, a 2′-O-methoxyethyl nucleotide, and a bicyclic sugar nucleotide;
the modified internucleoside linkage is a phosphorothioate internucleoside linkage; and/or
the first two internucleoside linkages at the 5′ terminus of the sense strand and the last two internucleoside linkages at the 3′ terminus of the sense strand are phosphorothioate internucleoside linkages;
the first two internucleoside linkages at the 5′ terminus of the antisense strand and the last two internucleoside linkages at the 3′ terminus of the antisense strand are phosphorothioate internucleoside linkages; and
the 5′-terminal modified phosphate group of the antisense strand is a 5′-(E)-vinylphosphonate.

221. The dsRNAi agent of claim 205, or a pharmaceutically acceptable salt thereof, wherein:
the antisense strand is 21 nucleotides in length and the nucleotides of the antisense strand are modified such that, counting from the 5′ terminus of the antisense strand, nucleotides 1, 3, 4, 5, 7, 8, 9, 11, 13, 15, 17, 18, 19, 20, and 21 are 2′-O-methyl nucleotides, nucleotides 2, 6, 10, 12, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkage is a phosphodiester internucleoside linkage; and
the sense strand is 19 nucleotides in length and the nucleotides of the sense strand are modified such that, counting from the 5′ terminus of the sense strand, nucleotides 5, 8, 9, 11, and 13 are 2′ fluoro nucleotides and nucleotides 1, 2, 3, 4, 6, 7, 10, 12, 14, 15, 16, 17, 18, and 19 are 2′-O-methyl nucleotides, the first two internucleoside linkages at the 5′ terminus and the last two internucleoside linkages at the 3′ terminus are phosphorothioate internucleoside linkages, and each other internucleotide is a phosphodiester internucleoside linkage.

222. The dsRNAi agent of claim 205, or a pharmaceutically acceptable salt thereof, wherein:
antisense strand is 21 nucleotides in length and the nucleotides of the antisense strand are modified such that, counting from the 5′ terminus of the antisense strand, nucleotides 1, 3, 4, 5, 7, 8, 9, 10, 11, 12, 13, 15, 17, 18, 19, 20, and 21 are 2′-O-methyl nucleotides, nucleotides 2, 6, 14, and 16 are 2′-fluoro nucleotides, the first two internucleoside linkages at the 5′ terminus and the last two internucleoside linkages at the 3′ terminus are phosphorothioate internucleoside linkages, and each other internucleoside linkages are phosphodiester internucleoside linkages; and
the sense strand is 19 nucleotides in length and the nucleotides of the sense strand are modified such that, counting from the 5′ terminus of the sense strand, nucleotides 5, 7, 8, and 9 are 2′-fluoro nucleotides and nucleotides 1, 2, 3, 4, 6, 10, 11, 12, 13, 14, 15, 16, 17, 18, and 19 are 2′-O-methyl nucleotides, the first two internucleoside linkages at the 5′ terminus and the last two internucleoside linkages at the 3′ terminus are phosphorothioate internucleoside linkages, and all other internucleoside linkages are phosphodiester internucleoside linkages.

223. The dsRNAi agent of claim 205, or a pharmaceutically acceptable salt thereof, wherein the dsRNAi agent is selected from a modified siRNA in Table 8, Table 9 or Table 11.

224. The dsRNAi agent of claim 205, wherein the pharmaceutical salt is a sodium salt.

225. The dsRNAi agent of claim 205, or a pharmaceutically acceptable salt thereof, comprising:
(i) an antisense strand, having a nucleotide sequence of
5′-
(SEQ ID NO: 1348)
X033U1027p001C007p001A004p000A004p000U004p000A007
p000A004p000A004p000U004p000A007p000U004p000C007
p000C004p000A007p000A004p000A007p000C004p000C004
p000G004p001C004p001C004-3′,
(ii) a sense strand, having a nucleotide sequence of
(SEQ ID NO: 1347)
5′-C004p001G004p001G004p000U004p000U007p000U004
p000G004p000G007p000A007p000U004p000A007p000U004
p000U007p000U004p000A004p000U004p000U004p001G004
p001A004p000X2140-3′,
wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

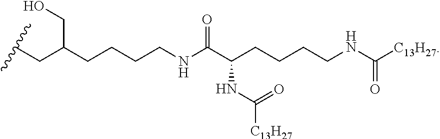

226. The dsRNAi agent of claim 205, or a pharmaceutically acceptable salt thereof, consisting of:
(i) an antisense strand, having a nucleotide sequence of
(SEQ ID NO: 1348)
5′-X033U1027p001C007p001A004p000A004p000U004
p000A007p000A004p000A004p000U004p000A007p000U004
p000C007p000C004p000A007p000A004p000A007p000C004
p000C004p000G004p001C004p001C004-3′,
(ii) a sense strand, having a nucleotide sequence of
(SEQ ID NO: 1347)
5′-C004p001G004p001G004p000U004p000U007p000U004
p000G004p000G007p000A007p000U004p000A007p000U004
p000U007p000U004p000A004p000U004p000U004p001G004
p001A004p000X2140-3′,
wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

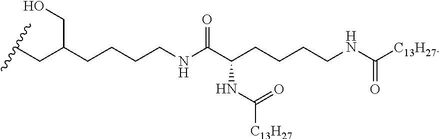

227. The dsRNAi agent of claim 205, or a pharmaceutically acceptable salt thereof, comprising:
an antisense strand having a nucleotide sequence of
(SEQ ID NO: 1352)
5′-X033U1027p001C007p001A004p000A004p000U004
p000A007p000A004p000A004p000U004p000A004p000U004
p000C004p000C004p000A007p000A004p000A007p000C004
p000C004p000G004p001C004p001C004-3′;
and
a sense stand strand having a nucleotide sequence of
(SEQ ID NO: 1351)
5′-C004p001G004p001G004p000U004p000U007p000U004
p000G007p000G007p000A007p000U004p000A004p000U004
p000U004p000U004p000A004p000U004p000U004p001G004
p001U004p000X2140-3′,
wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

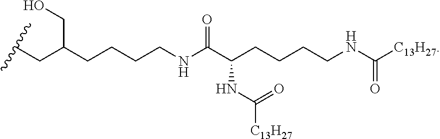

228. The dsRNAi agent of claim 205, or a pharmaceutically acceptable salt thereof, consisting of:
an antisense strand having a nucleotide sequence of
(SEQ ID NO: 1352)
5′-X033U1027p001C007p001A004p000A004p000U004
p000A007p000A004p000A004p000U004p000A004p000U004
p000C004p000C004p000A007p000A004p000A007p000C004
p000C004p000G004p001C004p001C004-3′;
and
a sense stand strand having a nucleotide sequence of
(SEQ ID NO: 1351)
5′-C004p001G004p001G004p000U004p000U007p000U004
p000G007p000G007p000A007p000U004p000A004p000U004
p000U004p000U004p000A004p000U004p000U004p001G004
p001U004p000X2140-3′,
wherein p000 is a phosphate internucleoside linkage, p001 is a phosphorothioate internucleoside linkage, X033U1027 is 5′-(E)-vinylphosphonate-2′-O-methyluridine, C004 is 2′-O-methylcytidine, C007 is 2′-fluorocytidine, A004 is 2′-O-methyladenosine, A007 is 2′-fluoroadenosine, U004 is 2′-O-methyluridine, U007 is 2′-fluorouridine, G004 is 2′-O-methylguanosine, G007 is 2′-fluoroguanosine, and X2140 is

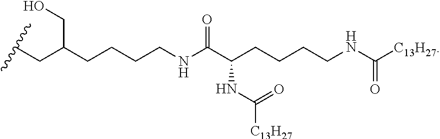

229. A pharmaceutical composition comprising a dsRNAi agent of claim 205, or a pharmaceutically acceptable salt thereof, and a pharmaceutically acceptable carrier.

230. The pharmaceutical composition of claim 229, wherein the pharmaceutically acceptable carrier is a sterile aqueous solution.

231. A method for treating myotonic dystrophy type I (DM1) in a subject in need thereof, the method comprising administering to the subject a therapeutically effective amount of a dsRNAi agent of claim 205, or a pharmaceutically acceptable salt thereof.

232. A method for delaying the onset of DM1 in a subject in need thereof, the method comprising administering to the subject a therapeutically effective amount of a dsRNAi agent of claim 205, or a pharmaceutically acceptable salt thereof.

233. A method for treating DM1 in a subject in need thereof, the method comprising administering to the subject a therapeutically effective amount of a dsRNAi agent of claim 205, or a pharmaceutically acceptable salt thereof, wherein the administration of the dsRNAi agent relieves at least one symptom of DM1 in at least one muscle.

234. The method of claim 233, wherein the at least one symptom of DM1 is myotonia.
